SEQUENCE LISTING (133 sequences)

SEQ ID NO: 1 - DNA, 434 bp, Arabidopsis thaliana
  misc_feature (1)..(434): 4905097_construct_ID_YP0103
atagcaaacaatcacatcatcgcaatatacataaacaaaagaggaagaaaaatggcaaccgagtggtgtagttatattgggaagaactcatggccggagcttttaggaacaaatggagactatgcggcttcggtgataaaaggagagaactcgagcctcaacgttgtcgtggtttcggatggaaattatgtgactgaagacctcagttgctaccgcgttagggtttgggttgacgaaatccgtatcgttgtcagaaacccaaccgccggctagacatgtatatggaccaccattatgctatagccatgtaggcgccttactatgaataaatgaaactatatataatgcatgcatagttggttggttggtcataatgtaacatctattgtttgcttgaatgattctggtgtccgatcatataacgcatttgaatg

SEQ ID NO: 2 - protein, 73 aa, Arabidopsis thaliana
  misc_feature (1)..(73): 4905097_protein_ID_4905099
MATEWCSYIGKNSWPELLGTNGDYAASVIKGENSSLNVVVVSDGNYVTEDLSCYRVRVWVDEIRIVVRNPTAG

SEQ ID NO: 3 - DNA, 711 bp, Arabidopsis thaliana
  misc_feature (1)..(711): 4906343_construct_ID_YP0098
acaaatcatttttcttaggatttgtttagtaaaataaaaatatttcttgtacatttcaatcataagtagatatggctaaatttaactctcagattactacgctattcattgttgtagctttggtgtgtgcatttgttccaactttctcagtcaaagaagctgaagcaaatttattatggaatacttgtcttgttaaattcactcctaagtgtgcgttagatataattgctgctgtcttcgaaaatggaacaatgtctgatccttgttgcaacgatcttgtcaaagaaggaaaagtgtgtcacgatacgcttattaaatatattgcagataaacccatgttaattgctcacgaaacagaatacttgaagaagagtgatgacttgtggaaacattgtgtctcaatctccaaaagtgcttgaaatgtatattgcgtgtactattttcacccaataaattgattgttttctgttgttatagttttcttcacacaagcctttatattttaacttaacaacaattttaaccaaagcgaatttctttcttaaaaagtataactttaatttatgattatctatttgaactcgaaacaaaatttcttataaagagtcgaataataattcaaaatttaactattaagaggagctctaactaatattgtttagtgaaatttaatttttgtattttctttctaattagagtaataagttattc

SEQ ID NO: 4 - protein, 115 aa, Arabidopsis thaliana
  misc_feature (1)..(115): 4906343_protein_ID_4906344
MAKFNSQITTLFIVVALVCAFVPTFSVKEAEANLLWNTCLVKFTPKCALDIIAAVFENGTMSDPCCNDLVKEGKVCHDTLIKYIADKPMLIAHETEYLKKSDDLWKHCVSISKSA

SEQ ID NO: 5 - DNA, 815 bp, Arabidopsis thaliana
  misc_feature (1)..(815): 4909291_construct_ID_YP0019
aattgtcttatctttcgacttttcttcttcttcttcttaagagatttttctccaagaaagttcgctccttttctctgttcttaacaaaaaagtctcggtttttttctctttgttttgggtactagcgtgatgtcttctgagaatgatttcgttgagttttcttctatgttcgagagaattatacaaggaagaggtgatggtctctctcgatttttgccggtgattgtagctttagccgccagagaagacgatgatgaccaaggatctaccgatcaaacaacgagacggggagatccgttgagtccaaggttcgtgatgatcggatcgcgatcgggactcgacgatttctttagcgacggtggaaaacaagggaggtcgccggcgttgaagtcagaagtggagaatatgccacgtgtcgtgatcggagaagataaggagaaatatggtggttcttgcgcgatttgtttggatgagtggtctaaaggtgacgtggcggcggagatgccttgtaaacataagtttcactcaaagtgtgtggaggagtggttagggaggcacgccacgtgtcctatgtgtaggtatgagatgcctgttgaagaagttgaagaagagaagaagattgggatttggattggtttctccattaacgccggcgacagaagaaactaagaagacggaggaagaagaagttaaaagtgactcgaaccctcaagatgcaacatggggctaggtttaggtttaggtttgctagaatgttttgtatagtttcgttttcgtttactgaaatcaatttcgaattcaataaaattggttgc

SEQ ID NO: 6 - protein, 179 aa, Arabidopsis thaliana
  misc_feature (1)..(179): 4909291_protein_ID_4909292
MSSENDFVEFSSMFERIIQGRGDGLSRFLPVIVALAAREDDDDQGSTDQTTRRGDPLSPRFVMIGSRSGLDDFFSDGGKQGRSPALKSEVENMPRVVIGEDKEKYGGSCAICLDEWSKGDVAAEMPCKHKFHSKCVEEWLGRHATCPMCRYEMPVEEVEEEKKIGIWIGFSINAGDRRN

SEQ ID NO: 7 - DNA, 1300 bp, Arabidopsis thaliana
  misc_feature (1)..(1300): 4909806_construct_ID_YP0050
gtcttggcatcctcgtcctcttcagcaaaactcgtctctcttgcactccaaaaagcaaccatgtctgcttttgtcggcaaatacgcagatgagctgataaagacggctaagtacattgccacaccgggaaagggcattttggcagcagacgagagcacgggaactattgggaaacgattcgccagcatcaatgttgagaacattgagtccaaccgccaagctctccgtgagctcctcttcacgtcccctggcactttcccttgcctctccggtgttatcctcttcgaggaaaccctctaccagaaaaccacggatggcaaacccttcgttgagctcctcatggaaaacggagttatccctggaatcaaagtggacaagggtgtggttgatctagcaggaaccaatggcgagaccactactcagggtctagattcacttggtgcacgttgccaggagtattacaaggcaggagctcggtttgcaaaatggcgtgcagtcctcaagattggggccaccgagccaagcgagctctctatccaagagaacgccaaggggctagcccgctatgccatcatctgccaggagaatggactcgtcccaatcgtcgagccagaggtactgaccgacgggagccatgacatcaagaaatgtgcagcggtgaccgagaccgttcttgctgccgtgtacaaggccttgaacgaccaccatgtcctcctcgaaggcactctgcttaaaccgaacatggtcactcccggctctgacagcccaaaggttgcaccggaagtgatagcggaatacacagtgactgctctgcgccgcacagtcccacctgcagttccaggaatcgtgttcctctcaggcggacagagtgaagaggaagcaacactaaatctgaacgcaatgaacaagctcgatgtgttgaagccatggactctcactttctcatttggccgagccctccaacaaagcactctcaaggcttgggcaggtaagacagagaatgtagccaaagctcaggccactttcctgaccaggtgcaagggtaactcggacgctaccctcgggaaatacaccggcggggcttctggtgactcggccgcctctgagagcttgtatgaggaaggatacaagtattaggagcgtttaaatacgggtgtcgccttttatacgatttgaatatatgtcaaatgtttcgtaggcgtttaactgtttaaatttttatcgatttggtttagcgtctgtgtaatgttcttaaactgtgttgtgttttttgtgatggtttctataatattttcgcgcc

SEQ ID NO: 8 - protein, 358 aa, Arabidopsis thaliana
  misc_feature (1)..(358): 4909806_protein_ID_4909808
MSAFVGKYADELIKTAKYIATPGKGILAADESTGTIGKRFASINVENIESNRQALRELLFTSPGTFPCLSGVILFEETLYQKTTDGKPFVELLMENGVIPGIKVDKGVVDLAGTNGETTTQGLDSLGARCQEYYKAGARFAKWRAVLKIGATEPSELSIQENAKGLARYAIICQENGLVPIVEPEVLTDGSHDIKKCAAVTETVLAAVYKALNDHHVLLEGTLLKPNMVTPGSDSPKVAPEVIAEYTVTALRRTVPPAVPGIVFLSGGQSEEEATLNLNAMNKLDVLKPWTLTFSFGRALQQSTLKAWAGKTENVAKAQATFLTRCKGNSDATLGKYTGGASGDSAASESLYEEGYKY

SEQ ID NO: 9 - DNA, 483 bp, Arabidopsis thaliana
  misc_feature (1)..(483): 4949423_construct_ID_YP0096
aacaaatactaatcattctttcttacgatttctttagtaaaataagaatatttcttgtatatttcaaccataagtagatatgtctaaatttaacactcagattactacattgttcattgttttagctttggtgtgtgcgtttgttccggctttctcagtcgaagaagctgaagcaacattattatggaatacttgtcttgttaaaatcactcctaagtgtgctttggatataatcgctgctgtctttgaaaatggaaccatgcctgatccttgttgcaaggatctcgtcaaagaaggaaaagtgtgtcacgatacgcttattaaatatattgcagataaacccatgttaattgcccacgaaacagaatacttgaagaagagtgatgacttgtggaaacattgtgtctcaatttccaaaagtgcttcaaatatggaatgcttttactattttgatttttgagccaaaaaattgatattttctgt

SEQ ID NO: 10 - protein, 126 aa, Arabidopsis thaliana
  misc_feature (1)..(126): 4949423_protein_ID_4949424
MSKFNTQITTLFIVLALVCAFVPAFSVEEAEATLLWNTCLVKITPKCALDIIAAVFENGTMPDPCCKDLVKEGKVCHDTLIKYIADKPMLIAHETEYLKKSDDLWKHCVSISKSASNMECFYYFDF

SEQ ID NO: 11 - DNA, 1316 bp, Arabidopsis thaliana
  misc_feature (1)..(1316): 5787483_construct_ID_YP0180
aacgccacaatcatggctttgttcttatctcctaaaaccatcactcttctcttcttctccctctccctcgcactctactgcagcatcgatcctttccaccactgcgccatttccgatttccccaatttcgtctctcacgaagttatctctccacgtcccgacgaagttccatgggagagagattcacaaaattcacttcagaaatcaaagattctgttttttaaccaaatccaaggtccagagagcgtcgcctttgattctctcggacgtggtccgtacacaggcgttgctgatggtagggttttgttttgggatggagagaaatggattgatttcgcttatacttcgagtaatcgatcggagatttgtgatccgaagccttctgctttgagttacttgaggaatgaacatatatgtggtcgtcctttaggtcttcgtttcgataagagaaccggagatttgtatatagctgatgcttatatgggacttttgaaagttggtcctgaaggtggtttagcaacgccgcttgtaactgaagctgaaggtgtgccgttggggtttactaatgatcttgacattgctgatgatggaactgtttactttacagatagcagcattagttaccagaggaggaacttcttgcagctcgttttctctggagacaatactgggagggttctaaagtatgatccagtagctaagaaagctgttgttttggtctcaaatcttcagtttccgaatggtgtctctatcagcagagacggttctttctttgtattctgcgaaggagatattggaagcctacgaagatactggttgaaaggcgagaaagctggaacgacagatgtgtttgcgtatttaccagggcatcctgataacgtaagaaccaaccaaaagggtgaattttgggtagcgcttcattgcagacgcaactactactcatacttaatggcaagatatcctaagctgaggatgttcatactgagactgccaatcactgcgagaactcactactcgttccagatagggttacggccgcacgggttggtggttaagtatagtcctgaagggaagcttatgcatgttttggaagatagtgaagggaaagttgtgagatcagtaagtgaagtggaagaaaaagatgggaagctttggatgggaagtgtgttgatgaactttgttgctgtctatgacctctgattacttgacctatacgtaaaccacttcactcagtttctagatttagcaaattcccaaaactgttaggtgtgtactgaaaaaatcaaacacttagcacaaacaaactcaatgttatt

SEQ ID NO: 12 - protein, 395 aa, Arabidopsis thaliana
  misc_feature (1)..(395): 5787483_protein_ID_5787485
MALFLSPKTITLLFFSLSLALYCSIDPFHHCAISDFPNFVSHEVISPRPDEVPWERDSQNSLQKSKILFFNQIQGPESVAFDSLGRGPYTGVADGRVLFWDGEKWIDFAYTSSNRSEICDPKPSALSYLRNEHICGRPLGLRFDKRTGDLYIADAYMGLLKVGPEGGLATPLVTEAEGVPLGFTNDLDIADDGTVYFTDSSISYQRRNFLQLVFSGDNTGRVLKYDPVAKKAVVLVSNLQFPNGVSISRDGSFFVFCEGDIGSLRRYWLKGEKAGTTDVFAYLPGHPDNVRTNQKGEFWVALHCRRNYYSYLMARYPKLRMFILRLPITARTHYSFQIGLRPHGLVVKYSPEGKLMHVLEDSEGKVVRSVSEVEEKDGKLWMGSVLMNFVAVYDL

SEQ ID NO: 13 - DNA, 1467 bp, Arabidopsis thaliana
  misc_feature (1)..(1467): 6795099_construct_ID_YP0095
atggccactggtgtttctgttgagaacataaaccccaaggttatactagggccatcatcgatcgctgagtgcatagtcattcgtggagaggttgccatccatgctcagcacctacaacagcagctacagacacaacctggttctcttccatttgatgagatcgtgtattgcaacatcgggaaccctcagtccttgggtcaaaaaccaatcacattcttcagggaggttcttgcactttgcaatcatccaaatctgctggagagagaggaaattaaatcattgttcagcactgatgctattgctcgggcaaagaaaattctttccatgattcctggaagagccaccggggcatatagtcatagccagggtatcaagggactgcgtgatgagattgctgctgggattgcctcccgtgatggtttccctgcaaatgcagatgatatattcctaactaatggagcaagtcctggtgtacacatgatgatgcagttgctgataaggaacaacagagatggcattatgtgtccaattcctcaatactcattgtactcagcatccctagcacttcatggcggagctcttgtgccatattatcttgatgaatcctcaggatggggtttggaggtttctaagcttaagaatcaacttgaagatgccaggtcaaaaggcataactgttagggcgttggtggtgatcaatcctggaaatcctactggacagattcttgatgagcaacagcaatatgagctagtaaagttctgcaaggacgaggaacttgttcttctggcggatgaggtataccaagagaacatttatgttaccaacaagaagatcaactctttcaagaagatagcaagatccatgggatacaatggagacgatttacaattagtatcattgcattctgtttctaaaggatattacggagagtgtggcaagagaggcggttacatggaggtcactggcttcagcactccagttagagaacaactctacaaaattgcatctgttaacttgtgttcaaatatcaccggccagatccttgcgagcctcataatggatccaccaaaggctggggacgcatcttatgacctctacgaggaagagaaagacaacatcctaaaatctttatctcgtcgtgcaaaggcaatggagtctgcatttaacagtattgatggaattacatgcaacaagacggaaggggcgatgtatctgttcccacggatttatctaccacagaaggcaattgaggctgccagggctgtcaacaaagcacctgatgtattctacgctctacgtcttcttgataccaccggcatcgttgtgactcctggatctggttttggacaagttgcagggacatggcacgtgagatgcacgatcctgccgcaggaggagaagataccttcgatgatctcccgcttcagggaattccatgaggagttcatgtcacagtatcgcgactga

SEQ ID NO: 14 - protein, 488 aa, Arabidopsis thaliana
  misc_feature (1)..(488): 6795099_protein_ID_6795100
MATGVSVENINPKVILGPSSIAECIVIRGEVAIHAQHLQQQLQTQPGSLPFDEIVYCNIGNPQSLGQKPITFFREVLALCNHPNLLEREEIKSLFSTDAIARAKKILSMIPGRATGAYSHSQGIKGLRDEIAAGIASRDGFPANADDIFLTNGASPGVHMMMQLLIRNNRDGIMCPIPQYSLYSASLALHGGALVPYYLDESSGWGLEVSKLKNQLEDARSKGITVRALVVINPGNPTGQILDEQQQYELVKFCKDEELVLLADEVYQENIYVTNKKINSFKKIARSMGYNGDDLQLVSLHSVSKGYYGECGKRGGYMEVTGFSTPVREQLYKIASVNLCSNITGQILASLIMDPPKAGDASYDLYEEEKDNILKSLSRRAKAMESAFNSIDGITCNKTEGAMYLFPRIYLPQKAIEAARAVNKAPDVFYALRLLDTTGIVVTPGSGFGQVAGTWHVRCTILPQEEKIPSMISRFREFHEEFMSQYRD

SEQ ID NO: 15 - DNA, 678 bp, Arabidopsis thaliana
  misc_feature (1)..(678): 12321680_construct_ID_YP0112
atattcttagtacaaataagaaattcacacccctcaaagaaatataacataatcaatcataggaaatatacttcgcataatgacgataatgatcaagtttctcctgttagctctgctcgtgatctctccgatttgcgccgagaaggacctgatgaaagaggaatgccataatgcacaagttccgaccatttgcatgcaatgtcttgaatccgacccaacctccgttcatgcagaccgtgttggcatcgccgagatcatcatacactgtctcgactctcgtctcgatatcatcaccaataacattacaaatatattgtcactgggaggaggaacgaaagaagtgagaaaaatcttggaggattgcagaaatgacacgtcgacggtggcacctaaactactgtcggaagccaaaacaggtctgaaaaccggtgattacgacaaagccgccaaatcgatagagtatgctagcattcctcatagctgtggattaaagcaaccaagtgtcgagtttgagtttcttcaactgtttagtcaaatcagtatctatactcaactctctgatgctgccatgagaatcattgatcgcttctaattactccacctttttatctctatgtaactcaacaacatcgatgcttaccatgcatcccccatataaataaatgattccctctttta

SEQ ID NO: 16 - protein, 170 aa, Arabidopsis thaliana
  misc_feature (1)..(170): 12321680_protein_ID_12321681
MTIMIKFLLLALLVISPICAEKDLMKEECHNAQVPTICMQCLESDPTSVHADRVGIAEIIIHCLDSRLDIITNNITNILSLGGGTKEVRKILEDCRNDTSTVAPKLLSEAKTGLKTGDYDKAAKSIEYASIPHSCGLKQPSVEFEFLQLFSQISIYTQLSDAAMRIIDRF

SEQ ID NO: 17 - DNA, 1003 bp, Arabidopsis thaliana
  misc_feature (1)..(1003): 12325134_construct_ID_YP0116
aactcaactcactcaaaccaaaaaaagaaacatcaaaccctaaaacacacataacaatcacaaatgaagaatccttcagtgatctcttttctcatcattctcctgtttgctgcaactatttgcacccacggaaatgaaccggtgaaggatacagccggaaatccacttaacacccgcgaacaatacttcatccagccggttaagaccgagagtaaaaacggaggtggtcttgtcccagccgccattacagtacttcccttttgtccacttggcatcacccaaacacttcttccctaccaacccggcctaccggttagcttcgtattagcacttggcgtaggatcaaccgttatgacatcttcggctgtaaacatcgagttcaagtccaacatctggccgttttgcaaggagttttccaagttttgggaagttgatgattcctcatcagctcccaaggagccttcaattctcatcggtggtaaaatgggggaccgaaatagctcgtttaagattgagaaagctggagaaggagctagagcaaacgtttataagttgaccaccttttacggaaccgttggagccatcccaggggtttggttaagcgcaccacaactaattatcaccaaggatacggctaagaccttactcgtcaaattcaaaaaggttgatgatgctactacggctactagcaacttatacttcccgggttgataatttaggtctaaggatgttcccgttctactaatcaactggtaaaaattattgtaatattaagcctgagactcgtccatggcctaaaataatgagttattttcaaatttcaattaataagaaagaaaaatgtggccagatccagatacatagatgttgagaatcattcataggcattgctgttgaatctgtttaaggcatgaaatagttttcttcttcattctactttgtatccgaaaattttctctcctcttgtaaagatcttgagcttgagaaaacattgatcattcat

SEQ ID NO: 18 - protein, 215 aa, Arabidopsis thaliana
  misc_feature (1)..(215): 12325134_protein_ID_12325135
MKNPSVISFLIILLFAATICTHGNEPVKDTAGNPLNTREQYFIQPVKTESKNGGGLVPAAITVLPFCPLGITQTLLPYQPGLPVSFVLALGVGSTVMTSSAVNIEFKSNIWPFCKEFSKFWEVDDSSSAPKEPSILIGGKMGDRNSSFKIEKAGEGARANVYKLTTFYGTVGAIPGVWLSAPQLIITKDTAKTLLVKFKKVDDATTATSNLYFPG

SEQ ID NO: 19 - DNA, 1563 bp, Arabidopsis thaliana
  misc_feature (1)..(1563): 12329827_construct_ID_YP0118
aatcatcatccaaaaacattcttctcacaagaatcagattcaagatagaagtttttcaaacaatgtctagtcctcttggtcactttcagattcttgtttttcttcatgctttgcttatcttctcagctgagtcccgcaaaacccaattgctgaacgataatgatgttgaatctagcgacaagagtgcaaaaggcacacgatgggctgttttagttgctggatcaaatgaatattataactacaggcatcaggctgacatatgccacgcgtatcagatactccgaaaaggcggtttaaaagatgaaaacatcattgtgtttatgtatgatgatatcgcgttttcctcggagaatcctaggcctggagttatcattaataaaccagatggagaagatgtttataaaggagttcctaaggactacactaaagaagctgttaatgttcaaaacttctacaatgtgttacttggaaatgaaagtggcgtcacaggaggaaatggcaaagttgtgaaaagtggtcctaatgataatatcttcatctattatgctgaccatggagctcctggcttaatagcgatgcccactggtgatgaagttatggcaaaagatttcaatgaagtcttggagaagatgcataagagaaaaaaatacaacaagatggtgatctatgttgaagcatgtgaatcaggaagtatgtttgaagggattttaaagaaaaatctcaacatatacgcagtgactgctgctaattctaaagagagcagctggggagtttactgtcctgagtcatatcctcctcctccttctgagattggaacttgtctcggcgatacatttagcatctcttggcttgaggacagtgaccttcatgacatgagcaaagagactttggagcaacaataccacgttgtaaagagaagagtaggatctgatgtaccagagacttctcatgtatgccgtttcggaacagagaagatgcttaaagattatctttcctcttacattggaagaaatcctgaaaacgataacttcactttcacggaatccttttcctcaccaatctctaattctggcttggtcaatccgcgcgatattcctctgctatacctccagagaaagattcaaaaagctccaatgggatcacttgaaagcaaagaagctcagaagaaattgcttgacgaaaagaatcataggaaacaaatcgatcagagcattacagacattctgcggctttcagttaaacaaaccaatgtcttaaatctcttaacttccacaagaacaacaggacagcctcttgtagacgattgggattgcttcaagactctagttaatagcttcaagaatcactgcggtgcaacggtgcattacggattgaagtatacaggagcgcttgccaatatctgcaatatgggagtggatgtgaagcaaactgtttcagccattgaacaagcttgttcgatgtaatgatttgcaaaacaatgtgatattcgactttaaaaatatcaaagttaatttcaataaaactcgatgtagagatggttggttcatgatactacttttacat

SEQ ID NO: 20 - protein, 466 aa, Arabidopsis thaliana
  misc_feature (1)..(466): 12329827_protein_ID_12329829
MSSPLGHFQILVFLHALLIFSAESRKTQLLNDNDVESSDKSAKGTRWAVLVAGSNEYYNYRHQADICHAYQILRKGGLKDENIIVFMYDDIAFSSENPRPGVIINKPDGEDVYKGVPKDYTKEAVNVQNFYNVLLGNESGVTGGNGKVVKSGPNDNIFIYYADHGAPGLIAMPTGDEVMAKDFNEVLEKMHKRKKYNKMVIYVEACESGSMFEGILKKNLNIYAVTAANSKESSWGVYCPESYPPPPSEIGTCLGDTFSISWLEDSDLHDMSKETLEQQYHVVKRRVGSDVPETSHVCRFGTEKMLKDYLSSYIGRNPENDNFTFTESFSSPISNSGLVNPRDIPLLYLQRKIQKAPMGSLESKEAQKKLLDEKNHRKQIDQSITDILRLSVKQTNVLNLLTSTRTTGQPLVDDWDCFKTLVNSFKNHCGATVHYGLKYTGALANICNMGVDVKQTVSAIEQACSM

SEQ ID NO: 21 - DNA, 718 bp, Arabidopsis thaliana
  misc_feature (1)..(718): 12332135_construct_ID_YP0113
atcaccaccaccaaatatcaaacgcaaaaacctattatcaaaagaactagggagaaatgactaatcccatgatcatggttatgctgttgttgtttcttgtgatgtcgactagagcagacgaagagctgattaagacagagtgtaatcacacagaataccaaaacgtatgcctcttctgtcttgaagccgatccaatctccttcaatatcgaccgtgctggacttgtcaacatcattatacactgtctcggatctcaacttgatgttcttatcaacaccgtcacgagtctaaagttgatgaaaggagagggtgaagcaaatgagaatgttctgaaagattgcgtcacaggctttgcgattgcacaattacgacttcaaggagccaacatcgatttgataacccttaattacgataaagcgtacgaattggtgaaaactgcgttaaactatcctcggacttgcgaagaaaatctccaaaaactcaagttcaaagattcatctgatgtttatgacgatatcttggcatatagccaactcacctctgttgctaagacgttgatccaccgtctctagatcaatatatatgtcgatctggttatcaaaaatatatttatgtcgatcgtttgctaccactaataaaataaaactccattatgtatgtcacgcgtgatttaatttcactcatcaacaaataaaataaaataaaataaaatgtttag

SEQ ID NO: 22 - protein, 171 aa, Arabidopsis thaliana
  misc_feature (1)..(171): 12332135_protein_ID_12332136
MTNPMIMVMLLLFLVMSTRADEELIKTECNHTEYQNVCLFCLEADPISFNIDRAGLVNIIIHCLGSQLDVLINTVTSLKLMKGEGEANENVLKDCVTGFAIAQLRLQGANIDLITLNYDKAYELVKTALNYPRTCEENLQKLKFKDSSDVYDDILAYSQLTSVAKTLIHRL

SEQ ID NO: 23 - DNA, 693 bp, Arabidopsis thaliana
  misc_feature (1)..(693): 12333534_construct_ID_YP0138
cacccatctccttctccataactctctctctctctccctaaacacaaccaaagacttttatctctcaggaaccccaaaaacaaatggctataatgaagaaaacttcaaaactcactcaaacagcaatgctgaagcagattctgaagagatgctcgagcttagggaagaagaatggaggagggtacgatgaagattgccttccgcttgacgtaccaaagggacacttccctgtctatgtcggagagaacagaagcagatacattgtcccaatctccttcttgacacatcctgagttccaatctctcttacaacgagccgaggaagaatttggattcgatcacgacatgggtctcaccattccttgtgatgaactcgtttttcaaaccctaacatccatgatccgatgatattttatcatttgaagaagaagcagaaggagatggttaaagaagaagcggaaaagcttctcatacaaaaaaagcatctcttctctttttttaagattttttttcctttatttttaagcccatctagggttttttttacgagttaattgactcgtctaactagaaataaatccgtatgagatagagattctatgggtttagatctgtaaataaagtttgtaatgttttcctcacagatcttcgttctgtgagagaagttatttaatgcaagagaaagtattcctcc

SEQ ID NO: 24 - protein, 107 aa, Arabidopsis thaliana
  misc_feature (1)..(107): 12333534_protein_ID_12333535
MAIMKKTSKLTQTAMLKQILKRCSSLGKKNGGGYDEDCLPLDVPKGHFPVYVGENRSRYIVPISFLTHPEFQSLLQRAEEEFGFDHDMGLTIPCDELVFQTLTSMIR

SEQ ID NO: 25 - DNA, 1591 bp, Arabidopsis thaliana
  misc_feature (1)..(1591): 12348737_construct_ID_YP0054
attttggttaaagcaaaagattttaagagagaaagggggagaagtgagagagatggagcataagagaggacatgtattagcagtgccgtacccaacgcaaggacacatcacaccattccgccaattctgcaaacgacttcacttcaaaggtctcaaaaccactctcgctctcaccactttcgtcttcaactccatcaatcctgacctatccggtccaatctccatagccaccatctccgatggctatgaccatgggggtttcgagacagctgactccatcgacgactacctcaaagactttaaaacttccggctcgaaaaccattgcagacatcatccaaaaacaccagactagtgataaccccatcacttgtatcgtctatgatgctttcctgccttgggcacttgacgttgctagagagtttggtttagttgcgactcctttctttacgcagccttgtgctgttaactatgtttattatctttcttacataaacaatggaagcttgcaacttcccattgaggaattgccttttcttgagctccaagatttgccttctttcttctctgtttctggctcttatcctgcttactttgagatggtgcttcaacagttcataaatttcgaaaaagctgatttcgttctcgttaatagcttccaagagttggaactgcatgagaatgaattgtggtcgaaagcttgtcctgtgttgacaattggtccaactattccatcaatttacttagaccaacgtatcaaatcagacaccggctatgatcttaatctctttgaatcgaaagatgattccttctgcattaactggctcgacacaaggccacaagggtcggtggtgtacgtagcattcggaagcatggctcagctgactaatgtgcagatggaggagcttgcttcagcagtaagcaacttcagcttcctgtgggtggtcagatcttcagaggaggaaaaactcccatcagggtttcttgagacagtgaataaagaaaagagcttggtcttgaaatggagtcctcagcttcaagttctgtcaaacaaagccatcggttgtttcttgactcactgtggctggaactcaaccatggaggctttgaccttcggggttcccatggtggcaatgccccaatggactgatcaaccgatgaacgcaaagtacatacaagatgtgtggaaggctggagttcgtgtgaagacagagaaggagagtgggattgccaagagagaggagattgagtttagcattaaggaagtgatggaaggagagaggagcaaagagatgaagaagaacgtgaagaaatggagagacttggctgtcaagtcactcaatgaaggaggttctacggatactaacattgatacatttgtatcaagggttcagagcaaataggtaactcacatacagtagcaaaggtccttctataatatcttgttttgtacgtctttcattcagcataatcttttgttgacttttcttatgttgtatgttcaaatccccatattgcttcttgttgtatgttcaaatccccatattgcttcttgttgacaataataataataaaaacaatgcaactttacc

SEQ ID NO: 26 - protein, 449 aa, Arabidopsis thaliana
  misc_feature (1)..(449): 12348737_protein_ID_12348739
MEHKRGHVLAVPYPTQGHITPFRQFCKRLHFKGLKTTLALTTFVFNSINPDLSGPISIATISDGYDHGGFETADSIDDYLKDFKTSGSKTIADIIQKHQTSDNPITCIVYDAFLPWALDVAREFGLVATPFFTQPCAVNYVYYLSYINNGSLQLPIEELPFLELQDLPSFFSVSGSYPAYFEMVLQQFINFEKADFVLVNSFQELELHENELWSKACPVLTIGPTIPSIYLDQRIKSDTGYDLNLFESKDDSFCINWLDTRPQGSVVYVAFGSMAQLTNVQMEELASAVSNFSFLWVVRSSEEEKLPSGFLETVNKEKSLVLKWSPQLQVLSNKAIGCFLTHCGWNSTMEALTFGVPMVAMPQWTDQPMNAKYIQDVWKAGVRVKTEKESGIAKREEIEFSIKEVMEGERSKEMKKNVKKWRDLAVKSLNEGGSTDTNIDTFVSRVQSK

SEQ ID NO: 27 - DNA, 1025 bp, Arabidopsis thaliana
  misc_feature (1)..(1025): 12370148_construct_ID_YP0033
attcccacttccacacatacacatatacaacagagcaagagagtcaatcaagtagagtgaagatggcaactaaacaagaagctttagccatcgatttcataagccaacaccttctcacagactttgtttccatggaaactgatcacccatctctttttaccaaccaacttcacaactttcactcagaaacaggccctagaaccatcaccaaccaatcccctaaaccgaattcgactcttaaccagcgtaaaccgcccttaccgaatctatccgtctcgagaacggtttcaacaaagacagagaaagaggaagaagagaggcactacaggggagtgagacgaagaccgtggggaaaatacgcggcggagattagggatccgaacaaaaagggttgtaggatctggcttgggacttacgacactgccgtggaagctggaagagcttatgaccaagcggcgtttcaattacgtggaagaaaagcaatcttgaatttccctctcgatgttagggttacgtcagaaacttgttctggggaaggagttatcggattagggaaacgaaagcgagataagggttctccgccggaagaggagaaggcggctagggttaaagtggaggaagaagagagtaatacgtcggagacgacggaggctgaggttgagccggtggtaccattgacgccgtcaagttggatggggttttgggatgtgggagcaggagatggtattttcagtattcctccgttatctccgacgtctcccaacttttccgttatctccgtcacttaaaacttcggaaaagtcaacgtacgatgacgttttcacttgcgtcactctcatgatttcatttattcttgtataatataaaggtagcggtagtgtgcaaatatcaaataagtagtttaattagtaccaatcattttattcattattttttttagtagaatatttggatgttgaaaatataaatttaattttgtatttgttgatgttataaatttattgattgtataaacattcttagtc

SEQ ID NO: 28 - protein, 241 aa, Arabidopsis thaliana
  misc_feature (1)..(241): 12370148_protein_ID_12370150
MATKQEALAIDFISQHLLTDFVSMETDHPSLFTNQLHNFHSETGPRTITNQSPKPNSTLNQRKPPLPNLSVSRTVSTKTEKEEEERHYRGVRRRPWGKYAAEIRDPNKKGCRIWLGTYDTAVEAGRAYDQAAFQLRGRKAILNFPLDVRVTSETCSGEGVIGLGKRKRDKGSPPEEEKAARVKVEEEESNTSETTEAEVEPVVPLTPSSWMGFWDVGAGDGIFSIPPLSPTSPNFSVISVT

SEQ ID NO: 29 - DNA, 488 bp, Arabidopsis thaliana
  misc_feature (1)..(488): 12396394_construct_ID_YP0056
ggtcccaaagaaaaatacgcacacctactcccttcattctctatcctctccactcataatatatacatctaaatgcaatctctccaatttgcacccaatttcttcgaatcaacttatcaatggcctcatcagctgcgatgttcatgctccctcttcctctaactcagcagataacaacaaacaatactctgcagactacagccacaccggaaccgtcagcctccatagttaaatgcctttttccggcgagaaactcatcggaaagttctgctcgttcgaagtttagtctttggctatttggcaatcccgctacgtatgacaagaggttccaagaagctattgaacttagttgcttgtgatggagatttggagatttttcctagtctttttcttgtgttttttaaatggacatattgtaatttcttcccaagtctcaccctccgctgtaatttatctaataatcaattcgatcaaagatgttccgactg

SEQ ID NO: 30 - protein, 79 aa, Arabidopsis thaliana
  misc_feature (1)..(79): 12396394_protein_ID_12396395
MASSAAMFMLPLPLTQQITTNNTLQTTATPEPSASIVKCLFPARNSSESSARSKFSLWLFGNPATYDKRFQEAIELSCL

SEQ ID NO: 31 - DNA, 1890 bp, Arabidopsis thaliana
  misc_feature (1)..(1890): 12561142_construct_ID_YP0028
atggatactctctttagactagtcagtctccaacaacaacaacaatccgatagtatcattacaaatcaatcttcgttaagcagaacttccaccaccactactggctctccacaaactgcttatcactacaactttccacaaaacgacgtcgtcgaagaatgcttcaactttttcatggatgaagaagacctttcctcttcttcttctcaccacaaccatcacaaccacaacaatcctaatacttactactctcctttcactactcccacccaataccatcccgccacatcatcaaccccttcctccaccgccgcagccgcagctttagcctcgccttactcctcctccggccaccataatgacccttccgcgttctccatacctcaaactcctccgtccttcgacttctcagccaatgccaagtgggcagactcggtccttcttgaagcggcacgtgccttctccgacaaagacactgcacgtgcgcaacaaatcctatggacgctcaacgagctctcttctccgtacggagacaccgagcaaaaactggcttcttacttcctccaagctctcttcaaccgcatgaccggttcaggcgaacgatgctaccgaaccatggtaacagctgcagccacagagaagacttgctccttcgagtcaacgcgaaaaactgtactaaagttccaagaagttagcccctgggccacgtttggacacgtggcggcaaacggagcaatcttggaagcagtagacggagaggcaaagatccacatcgttgacataagctccacgttttgcactcaatggccgactcttctagaagctttagccacaagatcagacgacacgcctcacctaaggctaaccacagttgtcgtggccaacaagtttgtcaacgatcaaacggcgtcgcatcggatgatgaaagagatcggaaaccgaatggagaaattcgctaggcttatgggagttcctttcaaatttaacattattcatcacgttggagatttatctgagtttgatctcaacgaactcgacgttaaaccagacgaagtcttggccattaactgcgtaggcgcgatgcatgggatcgcttcacgtggaagccctagagacgctgtgatatcgagtttccgacggttaagaccgaggattgtgacggtcgtagaagaagaagctgatcttgtcggagaagaagaaggtggctttgatgatgagttcttgagagggtttggagaatgtttacgatggtttagggtttgcttcgagtcatgggaagagagttttccaaggacgagcaacgagaggttgatgctagagcgtgcagcgggacgtgcgatcgttgatcttgtggcttgtgagccgtcggattccacggagaggcgagagacagcgaggaagtggtcgaggaggatgaggaatagtgggtttggagcggtggggtatagtgatgaggtggcggatgatgtcagagctttgttgaggagatataaagaaggtgtttggtcgatggtacagtgtcctgatgccgccggaatattcctttgttggagagatcagccggtggtttgggctagtgcgtggcggccaacgtaaagggttgtttttattttttcataaggaattcgcaagttcgatttttacttgagatggtttcacacgtgtggtgatggttgatgatgggctttgagattgagagagttacgattatgatgataatgcagttcataatatgaatttggattttggaataggactaattaagtaattctgatcattgaggtgggtatcaaggttcatacaattcgtgattttttgttttgtctttggtatttattaattttaaaaatccattttggaatgaaatttgtgattacttttgtttatccg

SEQ ID NO: 32 - protein, 531 aa, Arabidopsis thaliana
  misc_feature (1)..(531): 12561142_protein_ID_12561143
MDTLFRLVSLQQQQQSDSIITNQSSLSRTSTTTTGSPQTAYHYNFPQNDVVEECFNFFMDEEDLSSSSSHHNHHNHNNPNTYYSPFTTPTQYHPATSSTPSSTAAAAALASPYSSSGHHNDPSAFSIPQTPPSFDFSANAKWADSVLLEAARAFSDKDTARAQQILWTLNELSSPYGDTEQKLASYFLQALFNRMTGSGERCYRTMVTAAATEKTCSFESTRKTVLKFQEVSPWATFGHVAANGAILEAVDGEAKIHIVDISSTFCTQWPTLLEALATRSDDTPHLRLTTVVVANKFVNDQTASHRMMKEIGNRMEKFARLMGVPFKFNIIHHVGDLSEFDLNELDVKPDEVLAINCVGAMHGIASRGSPRDAVISSFRRLRPRIVTVVEEEADLVGEEEGGFDDEFLRGFGECLRWFRVCFESWEESFPRTSNERLMLERAAGRAIVDLVACEPSDSTERRETARKWSRRMRNSGFGAVGYSDEVADDVRALLRRYKEGVWSMVQCPDAAGIFLCWRDQPVVWASAWRPT

SEQ ID NO: 33 - DNA, 1721 bp, Arabidopsis thaliana
  misc_feature (1)..(1721): 12576899_construct_ID_YP0020
aaccaaagactctttaccatctctttctctctctgtttgaagacatagcacaaaaaaaaaaaaaaagacagagcaaaaaaacacacaaagatgggcataatgatgatgattttgggtcttcttgtgatcattgtttgtttatgtactgctcttctccgatggaaccagatgcgatattctaagaaaggtcttcctcctggaaccatgggctggccaatatttggtgaaacgactgagtttcttaaacaaggaccagatttcatgaaaaaccaaagactaagatatgggagtttcttcaagtctcacattcttggttgcccaacaatagtctcaatggacgcagagttaaacagatacattctaatgaatgaatcgaaaggacttgttgccggttacccgcaatctatgcttgatattctagggacatgcaacatagctgcggttcatggcccgagccaccggctaatgagaggctcgttgctttctttaataagcccaaccatgatgaaagaccatctcttgcctaagattgatgatttcatgagaaactatctttgtggttgggatgatcttgagacagttgatatccaagaaaagaccaaacatatggcatttttatcatcgttgttacaaatagctgagactttgaaaaaaccagaggttgaagaatatagaacagagtttttcaagcttgttgtgggaactctatcggtcccgatcgatatcccgggaacgaattaccgcagtggagtccaagcaagaaacaacatcgataggttattgacagaactgatgcaagaaagaaaagagtctggagaaactttcacagacatgttgggttacttgatgaagaaggaagataaccgatacttgttaaccgataaagagataagagatcaagtggtaacgatcttgtattccggttatgagactgtctctacaacctccatgatggctcttaagtatctccatgatcatccaaaagctcttgaagaactcagaagagaacatttggctataagggagagaaaacgacctgacgaaccgctcactctcgacgatattaaatcgatgaaattcactcgagctgtgatctttgagacatcaagattggcaacgattgttaatggtgtccttaggaaaactactcacgacttagaactcaacggttatttaatcccaaaaggttggagaatttacgtatacacaagagagattaactatgatacatctctttatgaagatccaatgatctttaacccatggagatggatggaaaagagcttagaatcaaagagctatttcttactctttggaggtggagttaggctttgccctggaaaggaactaggaatctcggaagtctcaagcttccttcactactttgttacaaaatatagatgggaagagaatggagaagacaaattaatggtctttccaagagtttctgcaccaaaaggataccatcttaagtgttcaccttactgactagttttgtcctaatattgaaaaatgtgtaaataaatctattaagggtcattttgtagggctaattaacctattttatctattaaatctctcaagatcatagaggagatggataatgtacagagagaaagagagaagaagaaaatggaatatagaaaaaaataaaatatttgaaatgttgagcttagtctcttatcttgtaaatttgtaacccataaatttttacatttcat

SEQ ID NO: 34 - protein, 465 aa, Arabidopsis thaliana
  misc_feature (1)..(465): 12576899_protein_ID_12576900
MGIMMMILGLLVIIVCLCTALLRWNQMRYSKKGLPPGTMGWPIFGETTEFLKQGPDFMKNQRLRYGSFFKSHILGCPTIVSMDAELNRYILMNESKGLVAGYPQSMLDILGTCNIAAVHGPSHRLMRGSLLSLISPTMMKDHLLPKIDDFMRNYLCGWDDLETVDIQEKTKHMAFLSSLLQIAETLKKPEVEEYRTEFFKLVVGTLSVPIDIPGTNYRSGVQARNNIDRLLTELMQERKESGETFTDMLGYLMKKEDNRYLLTDKEIRDQVVTILYSGYETVSTTSMMALKYLHDHPKALEELRREHLAIRERKRPDEPLTLDDIKSMKFTRAVIFETSRLATIVNGVLRKTTHDLELNGYLIPKGWRIYVYTREINYDTSLYEDPMIFNPWRWMEKSLESKSYFLLFGGGVRLCPGKELGISEVSSFLHYFVTKYRWEENGEDKLMVFPRVSAPKGYHLKCSPY

SEQ ID NO: 35 - DNA, 1950 bp, Arabidopsis thaliana
  misc_feature (1)..(1950): 12646933_construct_ID_YP0121
attatattttgttaagtccactcttctctctcatatcttctaaccaaaacagagtcacaaggggctcttaagcccttccaactaaattcttttcttttgttctcttgaaactgaatccaccagacaaaaaaatgggggttgatggtgaactgaaaaagaagaaatgcatcattgctggggttatcacagccttgctcgttctcatggttgtcgctgttggcatcacaacatcaagaaacaccagtcattcagaaaaaatcgtccctgtgcagattaaaacagccaccacggcagttgaagcagtttgtgcacctactgattacaaagagacttgtgtcaatagtctcatgaaagcttctcctgactctactcagcctcttgatctcattaagcttggcttcaacgtcaccattcgatccatagaagatagcatcaagaaagcttccgtggagctgacagccaaggcagctaatgacaaggataccaaaggggctttggagttgtgtgagaagcttatgaatgatgctacagatgatctgaagaagtgtcttgataactttgatgggttctcaattcctcagattgaggactttgtcgaagatcttcgtgtttggcttagtggctccattgcttatcaacaaacatgtatggatacgtttgaagaaactaactcgaaactttcacaagacatgcagaaaatctttaaaacatctagagaactcactagtaatggccttgccatgattactaacatctctaaccttctcggagagttcaacgtcacaggagtaaccggggatctcggtaaatacgcaagaaaacttttgtcggcggaagacggtataccaagttgggttggaccaaacactagacggctcatggcaacgaaaggaggtgtgaaagctaacgtggtggttgcacacgacggaagtggtcagtacaagactatcaatgaagccttgaatgcagtgcctaaagccaaccaaaagccatttgttatctacattaagcaaggtgtctataacgagaaagttgacgtcaccaagaaaatgactcatgtcactttcatcggtgatggaccaaccaaaactaagatcactggtagtctcaactattacattggcaaggtcaagacataccttactgccactgttgcgatcaatggtgataacttcacggcgaagaacatcgggtttgaaaacactgcaggtcccgaaggacatcaagctgtggccctaagagtctcggcggatttggccgtcttctacaactgccaaatcgatggttaccaagacacactctacgtccattctcatcgtcaattcttccgtgactgcacagtctcgggcaccgttgacttcattttcggcgatggtatagtagtcttacaaaactgtaacattgttgtgagaaaacccatgaaaagtcagtcttgcatgatcacagcccaaggccgctccgataaacgtgaatccaccggactcgtgctacaaaactgccatattaccggagaaccagcgtatattcccgtaaaatctataaacaaagcatatcttggaaggccatggaaagagttttcaagaaccattataatgggaacaaccatagacgacgttattgatccagcgggatggcttccttggaatggtgattttgcacttaatacgctttactatgctgagtatgagaataatgggcctgggtcaaaccaagcccaacgtgttaagtggcctggaattaagaaactatcgcccaagcaagctcttcgatttactcctgctaggtttttacgtggtaacttgtggattccaccaaatcgtgtgccttacatggggaattttcagtagattccaattggtgaattttccactttctgtgtgctctttaaaaaaaaaaatgaaggtgaataatttatatgcgtgtcttgtcttaaagtcctgacttgccgaa

SEQ ID NO: 36 - protein, 571 aa, Arabidopsis thaliana
  misc_feature (1)..(571): 12646933_protein_ID_12646934
MGVDGELKKKKCIIAGVITALLVLMVVAVGITTSRNTSHSEKIVPVQIKTATTAVEAVCAPTDYKETCVNSLMKASPDSTQPLDLIKLGFNVTIRSIEDSIKKASVELTAKAANDKDTKGALELCEKLMNDATDDLKKCLDNFDGFSIPQIEDFVEDLRVWLSGSIAYQQTCMDTFEETNSKLSQDMQKIFKTSRELTSNGLAMITNISNLLGEFNVTGVTGDLGKYARKLLSAEDGIPSWVGPNTRRLMATKGGVKANVVVAHDGSGQYKTINEALNAVPKANQKPFVIYIKQGVYNEKVDVTKKMTHVTFIGDGPTKTKITGSLNYYIGKVKTYLTATVAINGDNFTAKNIGFENTAGPEGHQAVALRVSADLAVFYNCQIDGYQDTLYVHSHRQFFRDCTVSGTVDFIFGDGIVVLQNCNIVVRKPMKSQSCMITAQGRSDKRESTGLVLQNCHITGEPAYIPVKSINKAYLGRPWKEFSRTIIMGTTIDDVIDPAGWLPWNGDFALNTLYYAEYENNGPGSNQAQRVKWPGIKKLSPKQALRFTPARFLRGNLWIPPNRVPYMGNFQ

SEQ ID NO: 37 - DNA, 1521 bp, Arabidopsis thaliana
  misc_feature (1)..(1521): 12656458_construct_ID_YP0107
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgggtggctctacgcaatgtcaaggcatcggcaaatagtccttggcaacattgcatcgatagatatccggttaaaattgattctgatttgtcaaagtcaaagactcatgtccaaaacggtcggtcctaa

SEQ ID NO: 38 - protein, 506 aa, Arabidopsis thaliana
  misc_feature (1)..(506): 12656458_protein_ID_12656459
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 39 - DNA, 3168 bp, Arabidopsis thaliana
  misc_feature (1)..(3168): 12660077_construct_ID_YP0049
tctagatgaatactataccgacgatgactacacacacaaggaaatatatatatcagctttcttttcacctaaaagtggtcccggtttagaatctaattcctttatctctcattttcttctgcttcacattcccgctagtcaaatgttaataagtgcacacaacgttttctcgaagcattagaatgtcctcctcttaattaatctccttctgattagattctcaatagagtttaaatttgttaatggagagatatattgggaccctcaaggcttctaattataccacgtttggcataattctctatcgtttggggccacatctttcacacttcattaccttatcaccaaaacataaaatcaatcaacttttttttgccttattgattgtgttggatccctccaaaattaaaacttgtgttccccacaaaagcttacccaatttcacttcaatcttaacaaataggaccaccactaccacgtacggtttgcatcatacaaaccacaaactccttcttcattacaattattatatcatctactaaaacctctttctccctctctctttcttgttcttagtgctaaattttctttgttcaggagaaatataatggacctcaagtattcagcatctcattgcaacttatcctcagacatgaagctcaggcgttttcatcagcatcgaggaaaaggaagagaagaagagtatgatgcttcttctctcagcttgaacaatctgtcaaaacttattcttcctccacttggtgttgctagctataaccagaatcacatcaggtctagtggatggatcatctcacctatggactcaagatacaggtgctgggaattttatatggtgcttttagtggcatactctgcgtgggtttacccttttgaagttgcatttctgaattcatcaccaaagagaaacctttgtatcgcggacaacatcgtagacttgttcttcgcggttgacattgtcttgacgtttttcgttgcttacatagacgaaagaacacagcttcttgtccgtgaacctaaacagattgcagtgaggtacctatcaacatggttcttgatggatgttgcatcaactataccatttgacgctattggatacttaatcactggcacatccacgttaaatatcacttgtaatctcttgggattacttagattttggcgacttcgaagagttaaacacctcttcactaggctcgagaaggacataagatatagctatttctggattcgctgctttcgacttctatcagtgacattgtttctagtgcactgtgctggatgcagttattacctaatagcagacagatatccacaccaaggaaagacatggactgatgcgatccctaatttcacagagacaagtctttccatcagatacattgcagctatatattggtctatcactacaatgaccacagtgggatatggagatcttcatgcaagcaacactattgaaatggtattcataacagtctacatgttattcaatcttggcctcactgcttaccttattggtaacatgactaatttggtcgtggaagggactcgtcgtaccatggaatttaggaatagcattgaagcagcgtcaaactttgttaacagaaacagattgcctcctagattaaaagaccagatattagcttacatgtgtttaaggtttaaagcagagagcttaaatcagcaacatcttattgaccagctcccaaaatctatctacaaaagcatttgtcaacatctttttcttccatctgttgaaaaagtttacctcttcaaaggcgtctcaagagaaatacttcttcttctggtttcaaaaatgaaggctgagtatataccaccaagagaggatgtcattatgcagaacgaagcgccggatgatgtttacataattgtgtcaggagaagttgagatcattgattcagagatggagagagagtctgttttaggcactctacgttgtggagacatatttggagaagttggagcactttgttgcagaccacaaagctacacttttcaaactaagtctttatcacagcttctccgactcaaaacatctttccttattgagacaatgcagattaaacaacaagacaatgccacaatgctcaagaacttcttgcagcatcacaaaaagctgagtaatttagacattggtgatctaaaggcacaacaaaatggcgaaaacaccgatgttgttcctcctaacattgcctcaaatctcatcgctgtggtgactacaggcaatgcagctcttcttgatgagctacttaaggctaagttaagccctgacattacagattccaaaggaaaaactccattgcatgtagcagcttctagaggatatgaagattgtgttttagtactcttaaagcacggttgcaacatccacataagagatgtgaatggtaatagtgctctatgggaagcaataatatcgaagcattacgagatattcagaatcctttatcatttcgcagccatatcggatccacacatagctggagatcttctatgtgaagcagcgaaacagaacaatgtagaagtcatgaaggctcttttaaaacaggggcttaacgtcgacacagaggatcaccatggcgtcacagctttacaggtcgctatggcggaggatcagatggacatggtgaatctcctggcgacgaacggtgcagatgtagtttgtgtgaatacacataatgaattcacaccattggagaagttaagagttgtggaagaagaagaagaagaagaacgaggaagagtgagtatttacagaggacatccattggagaggagagaaagaagttgcaatgaagctgggaagcttattcttcttcctccttcacttgatgacctcaagaaaattgcaggagagaagtttgggtttgatggaagtgagacgatggtgacgaatgaagatggagctgagattgacagtattgaagtgattagagataatgacaaactctactttgtcgtaaacaagataatttagaagttgaaaaattataacgaaatgaagtttgagataagagagagcgtgacaaaaaaatgaaaaacaaattgtaatatttatatgcgtccatcaaagtgagatgtaacacatatttgggtaagaaacgttccaaatccctgacgtagctcgag

SEQ ID NO: 40 - protein, 802 aa, Arabidopsis thaliana
  misc_feature (1)..(802): 12660077_protein_ID_12660078
MDLKYSASHCNLSSDMKLRRFHQHRGKGREEEYDASSLSLNNLSKLILPPLGVASYNQNHIRSSGWIISPMDSRYRCWEFYMVLLVAYSAWVYPFEVAFLNSSPKRNLCIADNIVDLFFAVDIVLTFFVAYIDERTQLLVREPKQIAVRYLSTWFLMDVASTIPFDAIGYLITGTSTLNITCNLLGLLRFWRLRRVKHLFTRLEKDIRYSYFWIRCFRLLSVTLFLVHCAGCSYYLIADRYPHQGKTWTDAIPNFTETSLSIRYIAAIYWSITTMTTVGYGDLHASNTIEMVFITVYMLFNLGLTAYLIGNMTNLVVEGTRRTMEFRNSIEAASNFVNRNRLPPRLKDQILAYMCLRFKAESLNQQHLIDQLPKSIYKSICQHLFLPSVEKVYLFKGVSREILLLLVSKMKAEYIPPREDVIMQNEAPDDVYIIVSGEVEIIDSEMERESVLGTLRCGDIFGEVGALCCRPQSYTFQTKSLSQLLRLKTSFLIETMQIKQQDNATMLKNFLQHHKKLSNLDIGDLKAQQNGENTDVVPPNIASNLIAVVTTGNAALLDELLKAKLSPDITDSKGKTPLHVAASRGYEDCVLVLLKHGCNIHIRDVNGNSALWEAIISKHYEIFRILYHFAAISDPHIAGDLLCEAAKQNNVEVMKALLKQGLNVDTEDHHGVTALQVAMAEDQMDMVNLLATNGADVVCVNTHNEFTPLEKLRVVEEEEEEERGRVSIYRGHPLERRERSCNEAGKLILLPPSLDDLKKIAGEKFGFDGSETMVTNEDGAEIDSIEVIRDNDKLYFVVNKII

SEQ ID NO: 41 - DNA, 2841 bp, Arabidopsis thaliana
  misc_feature (1)..(2841): 12661844_construct_ID_YP0092
atggccgaggatttggacaagccattgctggatcctgatactttcaacagaaaaggaattgatttgggtatattgccgttggaggaggtttttgaatacctaagaacatcgcctcaagggcttttatctggagatgctgaagagagattgaagatatttggtcctaacagacttgaagagaaacaggagaacagatttgtgaaattcttaggttttatgtggaatcccttgtcatgggttatggaagctgctgcattgatggccattgccctcgctaatagtcaaagtctaggtcctgactgggaagactttactggaatcgtttgccttttgctgatcaacgcaacaatcagcttctttgaagaaaacaatgctgggaatgctgctgcagctcttatggctcgcttggctttaaaaacaagagttcttagagatggacagtggcaagaacaagatgcttctatcttggtacctggtgatataattagcattaagcttggggatatcattcctgcagatgctcgccttcttgaaggagaccccttgaagattgatcagtcagtgctgaccggagaatcactacctgtgaccaagaagaagggtgaacaggtcttttctggctctacttgtaaacaaggtgaaatagaagctgttgtgatagcaactggatcgaccaccttctttggaaaaacagcacgcttggtggacagtacagatgtaactggacattttcagcaggttcttacatcgattggaaacttctgcatttgctccattgctgttggaatggttcttgaaatcattatcatgttccctgtacaacatcgctcttacagaattgggatcaataatcttcttgtactactgattggagggatacccattgccatgcccactgtactatctgtaacgcttgccattggatctcatcgactttcacaacagggtgccattacgaaaagaatgaccgcaatagaggaaatggctgggatggatgtactctgctgtgataaaactggaacccttactttgaacagtcttaccgttgataaaaatcttattgaggtattcgttgactacatggacaaggatacaattttgttgcttgcaggccgagcttcacgactagaaaatcaggatgctatagatgcagccattgttagcatgcttgcagatcccagagaggcacgtgcaaacattagagaaatccatttcttaccattcaatcctgtggacaaacgtactgcaataacgtatattgattccgatggaaaatggtatcgtgctaccaaaggtgctcctgaacaggttctaaacttgtgtcagcagaaaaatgagattgcgcaaagagtttatgccatcattgatagatttgcagaaaaaggtttgaggtctcttgcggttgcttatcaggaaattccagagaaaagcaacaacagtcctggaggaccatggaggttctgtggtctgttgccactgtttgatcccccaaggcatgatagcggtgaaaccatccttagagctcttagcctgggagtttgcgttaagatgatcactggtgatcaattggcgattgcaaaggagacaggcagacgtcttggaatgggaaccaacatgtatccttcttcctctttgttaggccacaacaatgatgagcatgaagccattccagtggatgagctaattgaaatggcagatggatttgctggagttttccctgaacataagtatgagattgtaaagattttacaagaaatgaagcatgtggttggaatgaccggagatggtgtgaatgatgctcctgctctcaaaaaagctgacatcggaatagctgtcgcagatgcaacagatgctgcaagaagttctgctgacatagtactaactgatcccggcttaagtgtaattatcagtgctgtcttgaccagcagagccattttccagcggatgaggaactatacagtatatgcagtctctatcaccatacgcatacttggttttacacttttagcgttgatatgggaatacgacttcccacctttcatggttctgataatcgcaatactcaatgacgggactatcatgactatttctaaagatcgagttaggccatctcctacacccgagagttggaagctcaaccagatatttgcgacaggaattgtcattggaacatatctagcattggtcaccgtcctgttttactggatcattgtttctaccaccttcttcgagaaacacttccatgtaaaatcaattgccaacaacagtgaacaagtgtcatccgcgatgtatctccaagtgagcatcatcagtcaggcactcatatttgtaacacgtagtcgaggctggtcattttttgaacgtcccgggactctcctgatttttgccttcattcttgctcaacttgcggctacattaattgctgtgtatgccaacatcagctttgctaaaatcaccggcattggatggagatgggcaggtgttatatggttatacagtctgatattttacatacctctagatgttataaagtttgtctttcactacgcattgagtggagaagcttggaatctcgtattggaccgtaagacagcttttacttacaagaaagattatgggaaagatgatggatcgcccaatgtaaccatctctcagagaagtcgttccgcagaagaactcagaggaagccgttctcgcgcttcttggatcgctgaacaaaccaggaggcgtgcagaaatcgccaggcttctagaggttcattcagtgtcaaggcatttagaatctgtgatcaaactcaaacaaattgaccaaaggatgatccgtgcagctcatactgtctaa

SEQ ID NO: 42 - protein, 946 aa, Arabidopsis thaliana
  misc_feature (1)..(946): 12661844_protein_ID_12661845
MAEDLDKPLLDPDTFNRKGIDLGILPLEEVFEYLRTSPQGLLSGDAEERLKIFGPNRLEEKQENRFVKFLGFMWNPLSWVMEAAALMAIALANSQSLGPDWEDFTGIVCLLLINATISFFEENNAGNAAAALMARLALKTRVLRDGQWQEQDASILVPGDIISIKLGDIIPADARLLEGDPLKIDQSVLTGESLPVTKKKGEQVFSGSTCKQGEIEAVVIATGSTTFFGKTARLVDSTDVTGHFQQVLTSIGNFCICSIAVGMVLEIIIMFPVQHRSYRIGINNLLVLLIGGIPIAMPTVLSVTLAIGSHRLSQQGAITKRMTAIEEMAGMDVLCCDKTGTLTLNSLTVDKNLIEVFVDYMDKDTILLLAGRASRLENQDAIDAAIVSMLADPREARANIREIHFLPFNPVDKRTAITYIDSDGKWYRATKGAPEQVLNLCQQKNEIAQRVYAIIDRFAEKGLRSLAVAYQEIPEKSNNSPGGPWRFCGLLPLFDPPRHDSGETILRALSLGVCVKMITGDQLAIAKETGRRLGMGTNMYPSSSLLGHNNDEHEAIPVDELIEMADGFAGVFPEHKYEIVKILQEMKHVVGMTGDGVNDAPALKKADIGIAVADATDAARSSADIVLTDPGLSVIISAVLTSRAIFQRMRNYTVYAVSITIRILGFTLLALIWEYDFPPFMVLIIAILNDGTIMTISKDRVRPSPTPESWKLNQIFATGIVIGTYLALVTVLFYWIIVSTTFFEKHFHVKSIANNSEQVSSAMYLQVSIISQALIFVTRSRGWSFFERPGTLLIFAFILAQLAATLIAVYANISFAKITGIGWRWAGVIWLYSLIFYIPLDVIKFVFHYALSGEAWNLVLDRKTAFTYKKDYGKDDGSPNVTISQRSRSAEELRGSRSRASWIAEQTRRRAEIARLLEVHSVSRHLESVIKLKQIDQRMIRAAHTV

SEQ ID NO: 43 - DNA, 2311 bp, Arabidopsis thaliana
  misc_feature (1)..(2311): 12664333_construct_ID_YP0030
attccaatctctcaagaaaatctacagttcctccaaataataataccctccctctaaggcaactaattttcagcaatcatgtccgggactattaatcccccggacggaggagggtccggtgcaagaaacccaccagtcgttcgtcagagagtgctagctcctccgaaagcgggtttactaaaggacatcaagtccgtggttgaagaaactttcttccatgatgctccgcttagggatttcaagggccaaaccccagctaaaaaagcgttgctcgggatccaggctgtcttcccgatcatcgggtgggccagagaatacactcttcgcaaatttagaggtgatctcatcgccggtctcaccattgctagtctttgtatccctcaggatatcggatatgcaaaactcgcgaatgtcgatccgaaatacggactttattcgagtttcgtgccaccgctgatttacgcgggcatggggagttctagggatattgcgataggaccagtcgctgtggtgtctcttcttgtgggaactttgtgccaggccgtgatcgacccaaagaaaaacccggaggattatctccgacttgtcttcactgccactttctttgctggcattttccaagccggcctcggatttctacggttgggattcttgatagactttctgtcgcatgcggccgtggttgggttcatgggaggagcagccatcacaatcgctctccaacagcttaagggctttcttggcatcaaaacatttaccaagaaaactgatattgtttctgtcatgcactccgtattcaaaaacgctgagcatgggtggaattggcaaactatagtcattggcgccagtttcttgacctttcttctcgtcaccaaattcattgggaagagaaacaggaaactattttgggttccggcaattgcgcctcttatttcagtcattatctctaccttctttgtcttcatttttcgtgctgataaacaaggagtccaaattgtgaaacatatagatcaaggaatcaatccgatttccgttcataagattttcttctccggaaaatatttcaccgaaggaatccgaatcggaggcattgcgggtatggtcgccttaacggaggctgtagcgattgcaagaacatttgcggcaatgaaagactatcaaattgatggaaacaaagagatgattgccctagggactatgaacgtcgtcggttcaatgacctcttgttacattgccacgggttcgttttcgcgatctgccgtgaacttcatggcgggagtcgaaacggcggtttcaaacatagttatggccatagttgtagctctaaccttagagttcatcacaccactcttcaagtacactccaaatgctatcctcgcggccatcattatatcggctgtcctcggtcttatcgatattgacgcagcgattctcatatggaggatcgataaactcgacttcttggcttgcatgggagctttcttaggagtcatcttcatctcggttgagatcggtctcttgatcgctgtggtgatctcttttgcaaagatattgcttcaagtgacgagaccaagaaccacggttctagggaagctgccaaattcgaatgtatatcggaacactctacagtatccggacgctgcccaaattcccggaatcttgatcatccgtgttgactcggccatctacttttccaactccaactatgtccgagaaagggcatcaagatgggtgcgagaggagcaagaaaatgctaaggaatatggcatgccggcaatcagatttgtgattattgagatgtcaccggttaccgatatcgataccagtggtatccactccatcgaagaacttctcaagagcctcgagaagcaagaaattcagttgattctagcaaatccaggaccagtggtgattgagaaactttatgcttcaaagttcgtcgaggagattggagagaaaaatatcttccttactgttggcgacgcggtcgcagtttgttctacggaagtggctgagcaacaaacttaatatcgtctattcatatacataaacacatccatatatgtatgtgtatatatatatgaaagaaactaatttaagaactatgggttattttcatttttttgagatgatatgatattatgtgtgtaatatatgcatgattgttgaatttgtttggttcacacaatggtgagatgggaacaaagtcgaacgtttgacttttatttttattttttaatctttcaaatgttattttctcgtgatttgtgtttcgtttgagatgatgaataaattgtattttcaacttata

SEQ ID NO: 44 - protein, 649 aa, Arabidopsis thaliana
  misc_feature (1)..(649): 12664333_protein_ID_12664334
MSGTINPPDGGGSGARNPPVVRQRVLAPPKAGLLKDIKSVVEETFFHDAPLRDFKGQTPAKKALLGIQAVFPIIGWAREYTLRKFRGDLIAGLTIASLCIPQDIGYAKLANVDPKYGLYSSFVPPLIYAGMGSSRDIAIGPVAVVSLLVGTLCQAVIDPKKNPEDYLRLVFTATFFAGIFQAGLGFLRLGFLIDFLSHAAVVGFMGGAAITIALQQLKGFLGIKTFTKKTDIVSVMHSVFKNAEHGWNWQTIVIGASFLTFLLVTKFIGKRNRKLFWVPAIAPLISVIISTFFVFIFRADKQGVQIVKHIDQGINPISVHKIFFSGKYFTEGIRIGGIAGMVALTEAVAIARTFAAMKDYQIDGNKEMIALGTMNVVGSMTSCYIATGSFSRSAVNFMAGVETAVSNIVMAIVVALTLEFITPLFKYTPNAILAAIIISAVLGLIDIDAAILIWRIDKLDFLACMGAFLGVIFISVEIGLLIAVVISFAKILLQVTRPRTTVLGKLPNSNVYRNTLQYPDAAQIPGILIIRVDSAIYFSNSNYVRERASRWVREEQENAKEYGMPAIRFVIIEMSPVTDIDTSGIHSIEELLKSLEKQEIQLILANPGPVVIEKLYASKFVEEIGEKNIFLTVGDAVAVCSTEVAEQQT

SEQ ID NO: 45 - DNA, 1735 bp, Arabidopsis thaliana
  misc_feature (1)..(1735): 12669615_construct_ID_YP0204
aaactcagtcattatatttatttttgttgtatttcaacgttcaatctctgaaaatgaaatatgcattgattcttgttctcttttttgttgtcttcatatggcaatcaagctcatcatcagcaaactcggagactttcacacaatgcctaacctcaaactccgaccccaaacatcccatctcccccgctatcttcttctccggaaatggctcctactcctccgtattacaagccaacatccgtaacctccgcttcaacaccacctcaactccgaaacccttcctcataatcgccgcaacacatgaatcccatgtgcaagccgcgattacttgcgggaaacgccacaaccttcagatgaaaatcagaagtggaggccacgactacgatggcttgtcatacgttacatactctggcaaaccgttcttcgtcctcgacatgtttaacctccgttcggtggatgtcgacgtggcaagtaagaccgcgtgggtccaaaccggtgccatactcggagaagtttattactatatatgggagaagagcaaaaccctagcttatcccgccggaatttgtcccacggttggtgtcggtggccatatcagtggtggaggttacggtaacatgatgagaaaatacggtctcaccgtagataataccatcgatgcaagaatggtcgacgtaaatggaaaaattttggatagaaaattgatgggagaagatctctactgggcaataaacggaggaggaggagggagctacggcgtcgtattggcctacaaaataaaccttgttgaagtcccagaaaacgtcaccgttttcagaatctcccggacgttagaacaaaatgcgacggatatcattcaccggtggcaacaagttgcaccgaagcttcccgacgagcttttcataagaacagtcattgacgtagtaaacggcactgtttcatctcaaaagaccgtcaggacaacattcatagcaatgtttctaggagacacgacaactctactgtcgatattaaaccggagattcccagaattgggtttggtccggtctgactgtaccgaaacaagctggatccaatctgtgctattctggacaaatatccaagttggttcgtcggagacacttctactccaaaggaatcaacccgtgaactacctcaagaggaaatcagattacgtacgtgaaccgatttcaagaaccggtttagagtcaatttggaagaaaatgatcgagcttgaaattccgacaatggctttcaatccatacggtggtgagatggggaggatatcatctacggtgactccgttcccatacagagccggtaatctctggaagattcagtacggtgcgaattggagagatgagactttaaccgaccggtacatggaattgacgaggaagttgtaccaattcatgacaccatttgtttccaagaatccgagacaatcgtttttcaattaccgtgatgttgatttgggtattaattctcataatggtaaaatcagtagttatgtggaaggtaaacgttacgggaagaagtatttcgcaggtaatttcgagagattggtgaagattaagacgagagttgatagtggtaatttctttaggaacgaacagagtattcctgtgttaccataagtgtatttatttgattattggttagtgaaatttgttgttgtataatgattatatgtcgtatttttatttattattagtaatttataaagtttgatatt

SEQ ID NO: 46 - protein, 527 aa, Arabidopsis thaliana
  misc_feature (1)..(527): 12669615_protein_ID_12669617
MKYALILVLFFVVFIWQSSSSSANSETFTQCLTSNSDPKHPISPAIFFSGNGSYSSVLQANIRNLRFNTTSTPKPFLIIAATHESHVQAAITCGKRHNLQMKIRSGGHDYDGLSYVTYSGKPFFVLDMFNLRSVDVDVASKTAWVQTGAILGEVYYYIWEKSKTLAYPAGICPTVGVGGHISGGGYGNMMRKYGLTVDNTIDARMVDVNGKILDRKLMGEDLYWAINGGGGGSYGVVLAYKINLVEVPENVTVFRISRTLEQNATDIIHRWQQVAPKLPDELFIRTVIDVVNGTVSSQKTVRTTFIAMFLGDTTTLLSILNRRFPELGLVRSDCTETSWIQSVLFWTNIQVGSSETLLLQRNQPVNYLKRKSDYVREPISRTGLESIWKKMIELEIPTMAFNPYGGEMGRISSTVTPFPYRAGNLWKIQYGANWRDETLTDRYMELTRKLYQFMTPFVSKNPRQSFFNYRDVDLGINSHNGKISSYVEGKRYGKKYFAGNFERLVKIKTRVDSGNFFRNEQSIPVLP

SEQ ID NO: 47 - DNA, 2417 bp, Arabidopsis thaliana
  misc_feature (1)..(2417): 12670159_construct_ID_YP0040
agcatccacacacactttgaatgctcaatcaaagcttcttcatagttaaacttccacacaacgtcaaaactcgagaagaagatgaaagagagagattcagagagttttgaatctctctcacatcaagttctcccaaacacttcaaattcaacacacatgatccagatggccatggccaactcaggttcatctgcagccgcacaagccggtcaagaccagcctgaccggtcaaagtggctgcttgactgtcctgaaccacctagcccgtggcatgagctcaaaagacaagtcaaaggctctttcctaaccaaagccaaaaagttcaagtcacttcaaaaacagcctttcccaaaacaaatcctctctgtcctccaagccattttcccaatcttcggttggtgcagaaactataaactcaccatgttcaagaacgatctcatggctggtttaaccctcgctagcctctgcattccgcagagcattggttatgcaactcttgcaaagcttgatcctcaatatggcctatatacgagtgtggtaccaccattgatatatgcattgatggggacatcaagagagatagcaatcggaccggtggctgtagtatctcttcttatatcttcaatgttgcagaaactcatcgatccagaaacagatcccttgggatacaagaaactggtcctaaccacaaccttcttcgccgggatcttccaagcttctttcggtttattcaggttagggtttctggtggattttctgtcgcacgcagccatagttgggttcatgggtggtgcagccattgtaattggactccaacagcttaaaggtttgcttggtatcactaacttcaccaccaacactgacattgtctctgttcttcgagctgtctggagatcttgtcaacaacaatggagccctcacactttcatcctcggatgttctttcctcagttttatccttattactcgcttcatcgggaagaagtataagaagctgttttggctaccggcaatagctccgttgatcgccgtggtagtgtcaacactaatggtgtttctgactaaagccgacgagcatggtgtgaagacagtgaggcacatcaaaggaggtcttaatccaatgtccattcaggatctcgactttaatactcctcatctcggacaaatcgctaaaatcggattaatcattgccattgttgctctaaccgaggcgattgcggtggggaggtcgttcgccggaataaaagggtacagactcgatggaaacaaagaaatggtggccattggatttatgaatgttctcggttccttcacatcttgttacgctgctactggttcattctctcggacggccgtgaattttgcggcaggatgtgagacagcaatgtccaacattgttatggcggttacggtgtttgtagcactcgagtgtctaacgaggcttctctactatactccaatcgccatcctcgcttcaataattctctcagcacttccgggactaatcaacattaacgaggctattcacatttggaaagtcgataaattcgattttcttgctctcattggagctttctttggtgttttgttcgcttccgttgagatcggacttcttgtcgcggtggttatttcgtttgccaagatcatactcatatcaattcgtccagggatagaaacgcttggaagaatgcccgggaccgatacttttacagatactaatcaatatcctatgacggttaagactcccggagtgttgatttttcgtgtcaagtctgcattgttgtgctttgccaatgccagttcaattgaggaaaggattatgggatgggtcgatgaggaagaagaagaagaaaacacaaagagcaatgccaagagaaagatcctctttgtagtccttgatatgtcaagtttgatcaacgtcgatacatcggggattactgctttgctggaactgcataacaaattaatcaaaactggtgttgagctagtgatcgttaacccgaaatggcaagtaatccacaagctgaatcaagcaaagttcgtcgacagaatcggtggcaaagtttacttgacgatcggcgaagctcttgatgcttgctttggattaaaagtttaagaaacagttttcaaaggaccagttgtgttacgggttattgcatgtgatgaatttatgtgagttgttgtgatttaaataatgtgatgcgtgcatgatcatgattaatatttaagtacgtatgtgtaatagagtgcttggtcgtgactgaataaagtcatgcaaactataatgtgaggatcgatgggtgtgtttgtaactcgatagatttggaaataatgtataatatatgtaagtttgagaattattggtgttttgtatgattgttgaaatgttatatagaatcagggatatattttttgggg

SEQ ID NO: 48 - protein, 677 aa, Arabidopsis thaliana
  misc_feature (1)..(677): 12670159_protein_ID_12670160
MKERDSESFESLSHQVLPNTSNSTHMIQMAMANSGSSAAAQAGQDQPDRSKWLLDCPEPPSPWHELKRQVKGSFLTKAKKFKSLQKQPFPKQILSVLQAIFPIFGWCRNYKLTMFKNDLMAGLTLASLCIPQSIGYATLAKLDPQYGLYTSVVPPLIYALMGTSREIAIGPVAVVSLLISSMLQKLIDPETDPLGYKKLVLTTTFFAGIFQASFGLFRLGFLVDFLSHAAIVGFMGGAAIVIGLQQLKGLLGITNFTTNTDIVSVLRAVWRSCQQQWSPHTFILGCSFLSFILITRFIGKKYKKLFWLPAIAPLIAVVVSTLMVFLTKADEHGVKTVRHIKGGLNPMSIQDLDFNTPHLGQIAKIGLIIAIVALTEAIAVGRSFAGIKGYRLDGNKEMVAIGFMNVLGSFTSCYAATGSFSRTAVNFAAGCETAMSNIVMAVTVFVALECLTRLLYYTPIAILASIILSALPGLININEAIHIWKVDKFDFLALIGAFFGVLFASVEIGLLVAVVISFAKIILISIRPGIETLGRMPGTDTFTDTNQYPMTVKTPGVLIFRVKSALLCFANASSIEERIMGWVDEEEEEENTKSNAKRKILFVVLDMSSLINVDTSGITALLELHNKLIKTGVELVIVNPKWQVIHKLNQAKFVDRIGGKVYLTIGEALDACFGLKV

SEQ ID NO: 49 - DNA, 696 bp, Arabidopsis thaliana
  misc_feature (1)..(696): 12678173_construct_ID_YP0068
gaaatccctaaaataggagggaaaatatattgatcgtagctagggttatcgactcttttgtcaacctctccatggactttttcggttttaacagacctcaggtctgcaaagaacacaaagtgctgaacctgtttgctgataatcctgagatgaaagcctttttcgagaagatattttatagttggtatatcgacgttgaaggattcgacacttcgcttcctgaggatgagatgaaggaggccttgactaatcatttcaagtcatgtggagtaatcgctatggtttctttccggagacaccctgaaaccgatgttgtcaacggccttgctactattaccatgatgggaaatgacgctgatgagaaggtgatgctacttaatggaagtgaattgggaggaaggaaacttgttgtcaaggccaaccctactcccagactgaaacttgaccatcttaaccttccctttggcggctcctctgtcccaggtacatcataagtttggagtctctttggtgttttcagatccagatacaatgcaacctgctttcttttcatcactcgttgggtccttatgaactgtgagacaatgaaaccccctttgggtctttctttctttgccatgtttaaatgtaagctccatatgtatgacgtttgtgtgtggatgattaaagtaagctctattatcattatctagtttg

SEQ ID NO: 50 - protein, 140 aa, Arabidopsis thaliana
  misc_feature (1)..(140): 12678173_protein_ID_12678174
MDFFGFNRPQVCKEHKVLNLFADNPEMKAFFEKIFYSWYIDVEGFDTSLPEDEMKEALTNHFKSCGVIAMVSFRRHPETDVVNGLATITMMGNDADEKVMLLNGSELGGRKLVVKANPTPRLKLDHLNLPFGGSSVPGTS

SEQ ID NO: 51 - DNA, 1903 bp, Arabidopsis thaliana
  misc_feature (1)..(1903): 12679922_construct_ID_G0013
atctaatatctctttctcaatttcggttccactttcctttcgtttgcaaaaacccatcccatcaaaaataaacaagagggcctaaagaagaatcctaaagactttacgggtcttgtttaggataaaagaaatgcctgccggtggattcgtcgtcggggatggccaaaaggcttatcccggcaaactcactccctttgttctcttcacttgcgttgttgctgccatgggcggtctcatcttcggatacgatatcggaatctccggtggtgtgacgtctatgccgtctttcctcaagcgattcttcccgtcggtgtatcggaaacaacaagaggacgcgtcaacgaaccagtactgtcagtacgatagcccgacgctaacgatgttcacatcgtctctatatctagcggcgctaatttcgtcgctggtggcttccaccgtgacaagaaagttcggacggcggctctcgatgctcttcggcggcatactcttctgcgccggagctctcatcaatggtttcgccaaacatgtttggatgctcatcgtcggtcgtatcttgcttggtttcggtatcggtttcgctaatcaggctgtgccactgtacctctctgagatggctccatacaaatacagaggagctttaaacattggtttccagctctcaattacaatcggaatcctcgtcgccgaagtgctaaactacttcttcgccaagatcaaaggcggttggggatggcggctcagtctcggaggcgcggtggttcctgccttgatcataaccatcggctccctcgtcctccctgacactcccaattcaatgatcgagcgtggccaacacgaagaagccaaaaccaagctcagacgaatccgtggtgtcgatgacgtcagccaagagtttgacgatttggtcgccgctagtaaagagtcgcagtcgatagagcacccgtggagaaacctcctccgccgcaagtaccgaccacatctcacaatggccgttatgattccgttctttcaacagctaaccggaatcaatgtgattatgttttacgctccggttttgttcaacaccattggtttcacgaccgatgcttctctcatgtccgctgtggtcactggctcggttaacgtggccgctacgcttgtttctatctacggtgttgacagatggggacgtcggtttctctttcttgaaggtggtacacaaatgcttatatgccaggctgtggttgcagcttgcataggggccaagtttggggtagacgggacccctggtgagctaccaaagtggtatgctatagtggttgtaacgttcatttgcatctatgtggcgggttttgcgtggtcgtggggcccactagggtggttagtaccgagtgaaatcttcccgttggagataaggtcggcggcgcagagtatcaccgtgtccgtgaacatgatcttcacgttcattatcgcgcaaatcttcttgacgatgctttgtcatttgaagtttgggttattccttgttttcgcctttttcgtggtggtgatgtcgatctttgtatacattttcttgccggagacgaaagggattccgatagaggagatgggtcaagtgtggaggtcacactggtattggtcaaggtttgtggaggatggtgagtatgggaatgcgcttgagatgggcaagaacagtaaccaagctggaacgaagcatgtttgatttatcattgtttttaatgagagttttaagaaagaaagaaaaaagatttgtaatttctaatgtcgtaaaggaaaaagtgtattagcctagatatttattggtgtttatataattcaataccacatgaagaaattatgcatatgattcttcgttaattgtctgtaattgttatactctttacttaaaccaagtgttttctctttg

SEQ ID NO: 52 - protein, 522 aa, Arabidopsis thaliana
  misc_feature (1)..(522): 12679922_protein_ID_12679923
MPAGGFVVGDGQKAYPGKLTPFVLFTCVVAAMGGLIFGYDIGISGGVTSMPSFLKRFFPSVYRKQQEDASTNQYCQYDSPTLTMFTSSLYLAALISSLVASTVTRKFGRRLSMLFGGILFCAGALINGFAKHVWMLIVGRILLGFGIGFANQAVPLYLSEMAPYKYRGALNIGFQLSITIGILVAEVLNYFFAKIKGGWGWRLSLGGAVVPALIITIGSLVLPDTPNSMIERGQHEEAKTKLRRIRGVDDVSQEFDDLVAASKESQSIEHPWRNLLRRKYRPHLTMAVMIPFFQQLTGINVIMFYAPVLFNTIGFTTDASLMSAVVTGSVNVAATLVSIYGVDRWGRRFLFLEGGTQMLICQAVVAACIGAKFGVDGTPGELPKWYAIVVVTFICIYVAGFAWSWGPLGWLVPSEIFPLEIRSAAQSITVSVNMIFTFIIAQIFLTMLCHLKFGLFLVFAFFVVVMSIFVYIFLPETKGIPIEEMGQVWRSHWYWSRFVEDGEYGNALEMGKNSNQAGTKHV

SEQ ID NO: 53 - DNA, 1273 bp, Arabidopsis thaliana
  misc_feature (1)..(1273): 12688453_construct_ID_YP0192
tcatattcacctaaaaatcaggtcccctctctttatatctctaacattcttatatcagatcatattttttggatttcttgttaagtaacaccaatcttttaaaagtgttttcaggttaatataaaagaataatgatgttttcggtgacggttgcgatccttgtttgtcttattggctacatttaccgatcatttaagcctccaccaccgcgaatctgcggccatcctaacggtcctccggttacttctccgagaatcaagctcagtgatggaagatatcttgcttatagagaatctggggttgatagagacaatgctaactacaagatcattgtcgttcatggcttcaacagctccaaagacactgaatttcccatccctaaggatgtaattgaggagcttgggatatactttgtgttctacgatagagcaggatatggagaaagtgatccacacccatcacgcactgttaagagtgaagcatacgacattcaagaactcgccgataaactcaagatcggaccaaagttctatgttcttggtatatcactaggtgcttactcggtttatagttgcctcaaatacattccccacagactagctggagcagtcttaatggttccatttgtgaactattggtggactaaagtgcctcaagaaaaattgagtaaagcgttggagctaatgccaaagaaagaccaatggacgtttaaagtggctcattatgttccgtggttgttatattggtggttgacccaaaaactatttccgtcttcgagtatggtcacggggaacaatgcgttatgcagcgacaaagatttggtcgtcataaagaagaaaatggagaatccacgccctggcttggaaaaagttagacaacaaggagaccatgaatgtcttcaccgggacatgatagccggattcgcgacatgggaattcgacccgactgaattagaaaatccgtttgcggaaggcgaaggatcggtccacgtttggcaagggatggaagacagaatcattccatacgaaattaatcgatatatatcagagaagcttccatggattaagtaccatgaggtcttaggttatggacatcttctaaacgccgaggaggagaaatgcaaagacattatcaaggcacttcttgtcaactgatgatcatctctacacaagatgccacgaaaaatatagcatatttaatagattttatttatggattataatattatagcatattataagtttgtaagtaagatgaaaaccacttgaaagtc

SEQ ID NO: 54 - protein, 340 aa, Arabidopsis thaliana
  misc_feature (1)..(340): 12688453_protein_ID_12688454
MMFSVTVAILVCLIGYIYRSFKPPPPRICGHPNGPPVTSPRIKLSDGRYLAYRESGVDRDNANYKIIVVHGFNSSKDTEFPIPKDVIEELGIYFVFYDRAGYGESDPHPSRTVKSEAYDIQELADKLKIGPKFYVLGISLGAYSVYSCLKYIPHRLAGAVLMVPFVNYWWTKVPQEKLSKALELMPKKDQWTFKVAHYVPWLLYWWLTQKLFPSSSMVTGNNALCSDKDLVVIKKKMENPRPGLEKVRQQGDHECLHRDMIAGFATWEFDPTELENPFAEGEGSVHVWQGMEDRIIPYEINRYISEKLPWIKYHEVLGYGHLLNAEEEKCKDIIKALLVN

SEQ ID NO: 55 - DNA, 619 bp, Arabidopsis thaliana
  misc_feature (1)..(619): 12692181_construct_ID_YP0097
catatccaacaacaaaaacataagctaagaaaacgaaactcaactaattttgttatcacccaaaaagaagttcaaacacaatggctttcgctttgaggttcttcacatgccttgttttaacggtgtgcatagttgcatcagtcgatgctgcaatctcatgtggcacagtggcaggtagcttggctccatgtgcaacctatctatcaaaaggtgggttggtgccaccttcatgttgtgcaggagtcaaaactttgaacagtatggctaaaaccacaccagaccgccaacaagcttgcagatgcatccagtccactgcgaagagcatttctggtctcaacccaagtctagcctctggccttcctggaaagtgcggtgttagcattccatatccaatctccatgagcactaactgcaacaacatcaagtgaaatggaagcttacgtcgtcgttttggcgttaagagtatggtttaccagaagtactagaataaaatacggctatatatcttagctgatattaccatgtatttgtttttgtctcaatgctttgtcttattttcatatcatatgttgtattgatgtgctaaaactatgataatagtaccttattagtcatcttc

SEQ ID NO: 56 - DNA, 1123 bp, Arabidopsis thaliana
  misc_feature (1)..(1123): 12703041_construct_ID_YP0007
acagagacaacaaactaaagttggtggtgatagagtgagagagaaacatggaaggcaaagaagaagacgtcaatgttggagccaacaagttcccagagagacagccgatcggtacggcggctcagacggagagcaaggactataaggaaccaccaccggcgccgtttttcgaacccggcgagctcaaatcttggtctttctacagagcagggatagctgagttcatagccactttccttttcctctacgtcaccgttttgacagtcatgggtgttaagagagctcccaatatgtgtgcctctgttggaatccaaggcatcgcttgggcttttggtggcatgatctttgctcttgtttactgtactgctggaatctcaggaggacatattaatccggcggtgacttttggtttgttcttggcgaggaagctatctttaaccagagctctgttctacatagtaatgcagtgccttggagctatatgtggtgctggtgtggttaaagggtttcaaccagggctgtaccagacgaatggcggtggagctaatgtggtggctcatggttacacaaagggttcaggtcttggtgcagagattgttggaacttttgttctggtttacactgttttctcagctactgatgctaagagaagtgccagagactctcatgtccctatcttggctccgcttccaattgggtttgctgtcttcttggtgcacttggctaccatcccaattactggaactggcattaacccggccaggagtctcggagctgccatcatctacaacaaggatcatgcttgggatgaccattggatcttctgggtcggtccattcattggtgctgcgcttgctgctctgtaccatcagatagtcatcagagctattcctttcaagtccaagacataaagtttcctacatattctctgatcatcatcaagctaagaatatatcaatctttaattctatatgctttcttcttgtttcctatgtcatgtgtgatgatctctatatgtaccactagagctttgatcttgtaacagtgtaaatgtgtaatctattatgtatcaatggcattgtatcttgtaacattaattatgtcaatggaagaatacattgtg

SEQ ID NO: 57 - protein, 287 aa, Arabidopsis thaliana
  misc_feature (1)..(287): 12703041_protein_ID_12703042
MEGKEEDVNVGANKFPERQPIGTAAQTESKDYKEPPPAPFFEPGELKSWSFYRAGIAEFIATFLFLYVTVLTVMGVKRAPNMCASVGIQGIAWAFGGMIFALVYCTAGISGGHINPAVTFGLFLARKLSLTRALFYIVMQCLGAICGAGVVKGFQPGLYQTNGGGANVVAHGYTKGSGLGAEIVGTFVLVYTVFSATDAKRSARDSHVPILAPLPIGFAVFLVHLATIPITGTGINPARSLGAAIIYNKDHAWDDHWIFWVGPFIGAALAALYHQIVIRAIPFKSKT

SEQ ID NO: 58 - DNA, 1064 bp, Arabidopsis thaliana
  misc_feature (1)..(1064): 12711515_construct_ID_YP0022
atctcacaccaaaacacaaagctctcatcttcttttagtttccaaactcacccccacaactttcatttctatcaaccaaacccaaatgggtccaagttcgagcctcaccaccatcgtggcgactgttcttcttgtgacattgttcggttcggcctacgcaagcaacttcttcgacgagtttgacctcacttggggtgaccacagaggcaaaatcttcaacggaggaaatatgctgtctttgtcgctggaccaggtttccgggtcaggtttcaaatccaaaaaagagtatttggtcggtcggatcgatatgcagctcaaacttgtcgccggaaactcggccggcaccgtcactgcttactacttgtcttcacaaggagcaacacatgacgagatagactttgagtttctaggtaacgagacagggaagccttatgttcttcacaccaatgtctttgctcaagggaaaggagacagagagcaacagttttatctctggttcgacccaaccaagaacttccacacttactccattgtctggagaccccaacacatcatattcttggtggacaatttacccattagagtgttcaacaatgcagagaagctcggcgttcctttcccaaagagtcaacccatgaggatctactctagcctgtggaatgcagacgattgggccacgagaggtggtctagtcaagactgactggtccaaggctcctttcacagcttactacagaggattcaacgctgcggcttgcacagcctcttcaggatgtgaccctaaattcaagagttcttttggtgatggtaaattgcaagtggcaaccgagctcaatgcttatggcaggaggagactcagatgggttcagaaatacttcatgatctataattattgctctgatctcaaaaggttccctcgtggattccctccagaatgcaagaagtccagagtctgatgaacacatattacctcatatttctctgcttgtttgatgcaattcttaaattcctctgttattccattgtacattgtcaagatcaataaagcattcctggtttcaaaat

SEQ ID NO: 59 - protein, 289 aa, Arabidopsis thaliana
  misc_feature (1)..(289): 12711515_protein_ID_12711517
MGPSSSLTTIVATVLLVTLFGSAYASNFFDEFDLTWGDHRGKIFNGGNMLSLSLDQVSGSGFKSKKEYLVGRIDMQLKLVAGNSAGTVTAYYLSSQGATHDEIDFEFLGNETGKPYVLHTNVFAQGKGDREQQFYLWFDPTKNFHTYSIVWRPQHIIFLVDNLPIRVFNNAEKLGVPFPKSQPMRIYSSLWNADDWATRGGLVKTDWSKAPFTAYYRGFNAAACTASSGCDPKFKSSFGDGKLQVATELNAYGRRRLRWVQKYFMIYNYCSDLKRFPRGFPPECKKSRV

SEQ ID NO: 60 - DNA, 547 bp, Arabidopsis thaliana
  misc_feature (1)..(547): 12713856_construct_ID_YP0126
aagtttctcacattttccaataaagcatctaacttacaattaaagacaatccatggcgatcagaatccctcgtgtgctgcaatcatcgaagcagattctccgacaagccaaactgttgtcatcatcttcttcttctagctctcttgatgttcccaaaggctacttagcggtttacgtaggagaacaaaatatgaagagatttgtagttccggtttcgtacttggaccagccttcatttcaagatctattaagaaaggcagaggaagagtttggatttgatcatccaatgggtggcctcacaatcccttgcagtgaagaaatttttattgatcttgcttctcgcttcaactgatcatgactcactcgataaccttacttttgtcattgatttttgtacattttgttttcccaattagttttcttcaagagatgagatgacttagaaacagcatctctccttgaaagtgaaacagagacttgtaacactctttttcctcacttacagtgagttggactcaaatctaatcaaaaccatcatttagtcatc

SEQ ID NO: 61 - protein, 99 aa, Arabidopsis thaliana
  misc_feature (1)..(99): 12713856_protein_ID_12713857
MAIRIPRVLQSSKQILRQAKLLSSSSSSSSLDVPKGYLAVYVGEQNMKRFVVPVSYLDQPSFQDLLRKAEEEFGFDHPMGGLTIPCSEEIFIDLASRFN

SEQ ID NO: 62 - DNA, 2322 bp, Arabidopsis thaliana
  misc_feature (1)..(2322): 12736079_construct_ID_YP0001
atgaaaacacaatcagcttcaccgttcttcttcgtctccttcttcttcttcttcttcttcttctcttctctgtttcttctctcctctgctttaaactctgatggagttctcttactgagtttcaaatactctgttcttcttgatcctctctctttattacaatcatggaactacgaccacgacaatccttgttcatggcgaggtgtgttgtgtaataacgattcaagagttgttactttatctctcccaaactctaacctcgttggttcgattccttccgatctgggtttcctccaaaacctccaaagtcttaatctttccaataattcactcaatgggtcattaccggttgagtttttcgccgccgataagctccggtttcttgatttatcaaataacttgatctccggcgagatccctgtatcaatcggaggtttacacaacctccagacgttaaatctctccgataacatcttcaccgggaaactaccagctaacttagcgtctcttggaagcttaacggaggtttctctgaagaacaactacttctccggcgagtttcccggcggcggatggagatcggttcagtatctagacatttcttcaaatctaatcaacggttcactcccacctgatttctccggcgacaatctccgatacctgaatgtctcgtataaccaaatctccggagagattcctccgaatgttggtgccggttttcctcaaaacgccaccgttgatttctccttcaacaatttaaccggttcaatcccagattctccggtttaccttaaccagaaatcaatttcgttttccggaaacccgggtttatgcggaggtccgacccgaaacccgtgtcccattccttcatctccggccaccgtctcgccaccaacctctacacctgcactcgcagctatacctaaatcaatcgggtctaatcgagaaaccgaaccgaacaacaactcaaatcctcgaaccgggttaagaccaggagttataatcggaatcatagtcggagatatcgccggaatcggaatcctcgctcttatcttcttctacgtttataaatacaaaaacaacaagacagtggagaagaagaacaatcatagcctagaagctcatgaagctaaagacacaacttcgttatcaccatcatcatcaacaactacatcttcttcatctccagaacaatcaagcagatttgcaaaatggtcatgtctccgtaagaatcaagaaaccgatgaaaccgaagaagaagacgaagaaaatcaacggtcaggagagattggagagaataagaaagggactttagtaaccattgatggaggagagaaagagcttgaagttgaaactttgcttaaggcttctgcttacattttaggagccactggttcgagtataatgtacaagactgttcttgaggacggtacggttctcgcggttcgtcggttaggtgagaatggtttgagtcaacaacgccggtttaaagactttgaggcacatattcgagctattggtaaattggttcacccgaatttggtacgtcttcgtggattctattggggcaccgacgagaaattggtcatttacgattttgttcctaacggcagtctcgtcaacgcccgttacaggaaaggagggtcttcgccgtgccatttaccgtgggagactcggctcaagatagtaaaaggtttggctcgtgggcttgcttacctccacgacaagaaacatgtgcacggtaacttgaagcctagtaacatactcttgggccaagatatggagcccaagatcggagatttcgggctcgaaaggcttctcgccggggatactagctataaccgagctagtggatcatctcggattttcagtagcaagcgattgacagcatcctcgcgtgaatttggtaccatcgggcccacaccgagcccaagtccaagctccgttgggcccatatctccctattgcgcacccgagtcgctccgcaatctcaaaccaaacccgaaatgggatgtgtttgggtttggagtgatcctcctcgagctgctcacgggaaaaatagtgtcgatagacgaggtgggggtaggaaatgggctgaccgtagaggacgggaaccgggcgctaataatggctgatgtagcgatccgctccgaattggaaggcaaagaggactttttacttggccttttcaaattgggatatagttgtgcatctcaaattccacaaaagagaccgaccatgaaagaggcgttagtagtgtttgaaagatatcctattagctcatcggctaagagtccatcgtaccattacggacactattaa

SEQ ID NO: 63 - protein, 773 aa, Arabidopsis thaliana
  misc_feature (1)..(773): 12736079_protein_ID_12736080
MKTQSASPFFFVSFFFFFFFFSSLFLLSSALNSDGVLLLSFKYSVLLDPLSLLQSWNYDHDNPCSWRGVLCNNDSRVVTLSLPNSNLVGSIPSDLGFLQNLQSLNLSNNSLNGSLPVEFFAADKLRFLDLSNNLISGEIPVSIGGLHNLQTLNLSDNIFTGKLPANLASLGSLTEVSLKNNYFSGEFPGGGWRSVQYLDISSNLINGSLPPDFSGDNLRYLNVSYNQISGEIPPNVGAGFPQNATVDFSFNNLTGSIPDSPVYLNQKSISFSGNPGLCGGPTRNPCPIPSSPATVSPPTSTPALAAIPKSIGSNRETEPNNNSNPRTGLRPGVIIGIIVGDIAGIGILALIFFYVYKYKNNKTVEKKNNHSLEAHEAKDTTSLSPSSSTTTSSSSPEQSSRFAKWSCLRKNQETDETEEEDEENQRSGEIGENKKGTLVTIDGGEKELEVETLLKASAYILGATGSSIMYKTVLEDGTVLAVRRLGENGLSQQRRFKDFEAHIRAIGKLVHPNLVRLRGFYWGTDEKLVIYDFVPNGSLVNARYRKGGSSPCHLPWETRLKIVKGLARGLAYLHDKKHVHGNLKPSNILLGQDMEPKIGDFGLERLLAGDTSYNRASGSSRIFSSKRLTASSREFGTIGPTPSPSPSSVGPISPYCAPESLRNLKPNPKWDVFGFGVILLELLTGKIVSIDEVGVGNGLTVEDGNRALIMADVAIRSELEGKEDFLLGLFKLGYSCASQIPQKRPTMKEALVVFERYPISSSAKSPSYHYGHY

SEQ ID NO: 64 - DNA, 1601 bp, Arabidopsis thaliana
  misc_feature (1)..(1601): 12739224_construct_ID_Bin2A2-28716-HY2
gtgcgctctcatatttctcacattttcgtagccgcaagactcctttcagattcttacttgcagctatgggtaaagagaagtttcacattaacattgtggtcattggtcatgttgattctggaaaatcgaccacaactggtcacttgatctataagcttggtggtattgacaagcgtgtcatcgagaggttcgagaaggaggctgctgagatgaacaagaggtccttcaagtacgcatgggtgttggacaaacttaaggccgagcgtgagcgtggtattaccatcgatattgctctatggaagttcgagaccaccaagtactactgcacagtcattgatgccccaggacatcgtgatttcatcaagaacatgattactggtacctcccaggctgattgtgctgttcttatcattgactccaccactggaggttttgaggctggtatctctaaggatggtcagacccgtgagcacgctcttcttgctttcacccttggtgtcaagcagatgatttgctgttgtaacaagatggatgccaccacccccaaatactccaaggctaggtacgatgaaatcatcaaggaggtgtcttcatacctgaagaaggtcggatacaaccctgacaaaatcccatttgtgccaatctctggattcgagggagacaacatgattgagaggtcaaccaaccttgactggtacaagggaccaactcttcttgaggctcttgaccagatcaacgagcccaagaggccatcagacaagccccttcgtcttccacttcaggatgtctacaagattggtggtattggaacggtgccagtgggacgtgttgagactggtatgatcaagcctggtatggttgttacctttgctcccacagggttgaccactgaggttaagtctgttgagatgcaccacgagtctcttcttgaggcacttcccggtgacaatgttggattcaatgtcaagaatgttgctgtcaaggatcttaagagaggatacgttgcctctaactccaaggatgatccagctaagggtgccgccaacttcacctcccaggtcatcatcatgaaccaccctggtcagattggtaacggttacgccccagttctcgattgccacacctctcacattgcagtcaagttctctgagatcttgaccaagattgacaggcgttctggtaaggagattgagaaggagcccaagtttttgaagaatggtgacgctggtatggttaagatgaccccaaccaagcccatggttgttgagactttctccgagtacccacctttgggacgtttcgctgttagggacatgaggcagaccgttgctgttggtgttattaagagcgtggacaagaaggacccaactggagccaaggtcaccaaggctgcagtgaagaagggtgccaaatgatgagactttcgttatgatcgactctcttatggttttctttggttcttaaaactttgatggcgtttgagcctttttcttttttctctttatttctgtgactttctctctccctcctttttggatatctctgagactttttattatggttttcaattatgcagtttccggataattttgcttgaaact

SEQ ID NO: 65 - protein, 449 aa, Arabidopsis thaliana
  misc_feature (1)..(449): 12739224_protein_ID_12739226
MGKEKFHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIIKEVSSYLKKVGYNPDKIPFVPISGFEGDNMIERSTNLDWYKGPTLLEALDQINEPKRPSDKPLRLPLQDVYKIGGIGTVPVGRVETGMIKPGMVVTFAPTGLTTEVKSVEMHHESLLEALPGDNVGFNVKNVAVKDLKRGYVASNSKDDPAKGAANFTSQVIIMNHPGQIGNGYAPVLDCHTSHIAVKFSEILTKIDRRSGKEIEKEPKFLKNGDAGMVKMTPTKPMVVETFSEYPPLGRFAVRDMRQTVAVGVIKSVDKKDPTGAKVTKAAVKKGAK

SEQ ID NO: 66 - DNA, 731 bp, Arabidopsis thaliana
  misc_feature (1)..(731): 13489977_construct_ID_YP0134
cagtcggttctcgagtcatcgccaaggacccacttcatcattttacaaaccaagcaagactaatccaacaaaaaaatagtccacaaaaagatttttacagatggcgattaacagatctttacttttgattcttcttttcatctctgtttctctatcgacggcgaggatcttacccggagagtttgttccagtcatcttctccggagagatccctcctgtttctaagtcggcggtggttggttgcggaggcgagcaggagaccaagacggaatattcttcttttgttcctgaagttgtcgccggaaagttcgggtccttggtgttgaatgctcttccgaaagggagtcgtccggggtctggacccagcaagaaaactaacgacgtcaagacttagcactattctttctagagttttctgtcctaattcttacttctttctttttttgttctttagagattctttgatttttcgttttcaaatagagattattgtaaatgttacatgtattacagaaatttacagtagaagtttaggaaaaatgaggattttatttggtaatgtaagtcgaaatgatcaagacttagactatcatcttgtatcgtttcatcaatatttctttgataaacgttaatcagctttttaatttctatgattatgtatcaattttatttagactaagaaagtcttttaagttaaacgcataaaagagtcaaggataccatttgaattt

SEQ ID NO: 67 - protein, 101 aa, Arabidopsis thaliana
  misc_feature (1)..(101): 13489977_protein_ID_13489978
MLFRKGVVRGLDPARKLTTSRLSTILSRVFCPNSYFFLFLFFRDSLIFRFQIEIIVNVTCITEIYSRSLGKMRILFGNVSRNDQDLDYHLVSFHQYFFDKR

SEQ ID NO: 68 - DNA, 1907 bp, Arabidopsis thaliana
  misc_feature (1)..(1907): 13491988_construct_ID_YP0016
gtctcctcttcggataatcctatccttctcttcctataaatacctctccactcttcctcttcctccaccactacaaccaccgcaacaaccaccaaaaaccctctcaaagaaatttcttttttttcttactttcttggtttgtcaaatatggtcagccatccaatggagaaagctgcaaatggtgcgtctgcgttggaaacgcagacgggtgagttagatcagccggaacggcttcgtaagatcatatcggtgtcttccattgccgccggtgtacagttcggttgggctttacagttatctctgttgactccttacgtgcagctactcggaatcccacataaatgggcttctctgatttggctctgtggtccaatctccggtatgcttgttcagcctatcgtcggttaccacagtgaccgttgcacctcaagattcggccgtcgtcgtcccttcatcgtcgctggagctggtttagtcaccgttgctgttttccttatcggttacgctgccgatataggtcacagcatgggcgatcagcttgacaaaccgccgaaaacgcgagccatagcgatattcgctctcgggttttggattcttgacgtggctaacaacaccttacaaggaccctgcagagctttcttggctgatttatcagcagggaacgctaagaaaacgcgaaccgcaaacgcgtttttctcgtttttcatggcggttggaaacgttttgggttacgctgcgggatcttacagaaatctctacaaagttgtgcctttcacgatgactgagtcatgcgatctctactgcgcaaacctcaaaacgtgttttttcctatccataacgcttctcctcatagtcactttcgtatctctctgttacgtgaaggagaagccatggacgccagagccaacagccgatggaaaagcctccaacgttccgtttttcggagaaatcttcggagctttcaaggaactaaaaagacccatgtggatgcttcttatagtcactgcactaaactggatcgcttggttccctttccttctcttcgacactgattggatgggccgtgaggtgtacggaggaaactcagacgcaaccgcaaccgcagcctctaagaagctttacaacgacggagtcagagctggtgctttggggcttatgcttaacgctattgttcttggtttcatgtctcttggtgttgaatggattggtcggaaattgggaggagctaaaaggctttggggtattgttaacttcatcctcgccatttgcttggccatgacggttgtggttacgaaacaagctgagaatcaccgacgagatcacggcggcgctaaaacaggtccacctggtaacgtcacagctggtgctttaactctcttcgccatcctcggtatcccccaagccattacgtttagcattccttttgcactagcttccatattttcaaccaattccggtgccggccaaggactttccctaggtgttctgaatctagccattgtcgtccctcagatggtaatatctgtgggaggtggaccattcgacgaactattcggtggtggaaacattccagcatttgtgttaggagcgattgcggcagcggtaagtggtgtattggcgttgacggtgttgccttcaccgcctccggatgctcctgccttcaaagctactatgggatttcattgaattttagcagtggttgtttggctctctttctctcataaaacagtagtgttgtgcaaatcctacataaagaaaaaagaaaaggaaattaaactcattgggttggtttgtattttacctaaacccacgaagttcctttttctttttgtaactcaatttaaatttggagtatattttactttttgttaccttcaaggcttcaatattacgacttcattgttcgg

SEQ ID NO: 69 - protein, 512 aa, Arabidopsis thaliana
  misc_feature (1)..(512): 13491988_protein_ID_13491989
MVSHPMEKAANGASALETQTGELDQPERLRKIISVSSIAAGVQFGWALQLSLLTPYVQLLGIPHKWASLIWLCGPISGMLVQPIVGYHSDRCTSRFGRRRPFIVAGAGLVTVAVFLIGYAADIGHSMGDQLDKPPKTRAIAIFALGFWILDVANNTLQGPCRAFLADLSAGNAKKTRTANAFFSFFMAVGNVLGYAAGSYRNLYKVVPFTMTESCDLYCANLKTCFFLSITLLLIVTFVSLCYVKEKPWTPEPTADGKASNVPFFGEIFGAFKELKRPMWMLLIVTALNWIAWFPFLLFDTDWMGREVYGGNSDATATAASKKLYNDGVRAGALGLMLNAIVLGFMSLGVEWIGRKLGGAKRLWGIVNFILAICLAMTVVVTKQAENHRRDHGGAKTGPPGNVTAGALTLFAILGIPQAITFSIPFALASIFSTNSGAGQGLSLGVLNLAIVVPQMVISVGGGPFDELFGGGNIPAFVLGAIAAAVSGVLALTVLPSPPPDAPAFKATMGFH

SEQ ID NO: 70 - DNA, 858 bp, Arabidopsis thaliana
  misc_feature (1)..(858): 13580795_construct_ID_YP0087
tttagggtttattcttcattgcttgagcttccttctcttcttcttcttcaagccgcggctaaagatccctacttctctcgacacttatagagtttcagtcatggccgcctccgcagaaatcgacgctgagattcaacagcagcttaccaatgaggttaagctcttcaaccgttggagctttgatgacgtttcggttacggatattagtcttgtggactacattggtgttcagccatcgaagcacgcaacttttgttccccatactgctggacgatactctgtgaagaggttcagaaaggcgcagtgcccaattgttgagaggctcactaactctctcatgatgcacggaagaaacaatggtaagaagttgatggctgtcaggatcgtcaagcatgccatggagattatccacctcttgtctgacttgaacccgattcaagttatcattgatgccattgttaacagtggtccacgtgaagatgctaccaggattggatctgctggtgtggttaggaggcaggctgttgatatctctcctctaagacgtgtgaaccaagcgatcttcttgcttacaactggtgcacgtgaagctgcctttagaaacatcaagacaatcgctgagtgccttgctgatgaactcatcaatgctgcaaagggatcttccaacagctatgccatcaagaagaaagatgagattgagagagttgctaaggccaatcgttaagggatctccctttcctctaagtttgcattatatcaaagagtttttgtgttgtttccattagctttggatatgtttcagatgatctctctatctttaatgaaattttgacgcttataatcgacttgggatcttga

SEQ ID NO: 71 - protein, 207 aa, Arabidopsis thaliana [sequence omitted]

SEQ ID NO: 72 - DNA, 1111 bp, Arabidopsis thaliana
  misc_feature (1)..(1111): 13601936_construct_ID_YP0108
atcataaacccaccgagacgatgtctctcatcatcgtcttcttcttcttctcactcttgctcacatccaatggacagttcttcgacgagagcaagaactatgaaggctcctccgatctcgttgaccttcaataccacttgggtccggtcatatcctcgccggtgacgagtctctacatcatttggtacggccgatggaacccaactcaccaatctataatccgagactttctctactctgtctctgcaccggcaccggctcagtacccgtcagtatccaactggtggaagacagtgaggctatacagagaccagacaggttccaacatcaccgacactcttgtcttatccggagagttccacgactcaacgtactctcatggatctcatctcactcgcttctctgttcagtctgtgatcagaactgccttgacttccaagttaccactaaacgctgtaaacggcttgtacttagtcttgacctcggatgatgtagagatgcaagagttctgcagagcgatttgcgggtttcattacttcactttcccaagcgttgtgggtgcaaccgtaccgtatgcttgggtgggcaacagtgagagacagtgtccagaaatgtgtgcgtacccatttgcacagcctaagccatttccggggagcgggtttgtagccagagagaagatgaaaccgccaaatggagaggtaggaatcgatgggatgatcagtgtgatagctcatgagctggcagaagtgtcgagtaacccgatgttaaacggatggtatggaggagaggacgcgacagcaccgacagagatagcggatttatgtttgggagtgtatgggtcaggaggaggaggaggctatatgggaagtgtgtataaggataggtggaggaatgtgtataatgtgaagggcgttaaaggaagaaagtatctaattcaatgggtttgggatcttaataggaacagatgctttggaccaaacgctatgaattagagactatcatgtttgttacctcttttcaccaaagccttgagcttgaagcttggggaaacctgtatatggtttatcttttccttgcctagtcgattctatgcatttgattgtttaagact

SEQ ID NO: 73 - protein, 323 aa, Arabidopsis thaliana
  misc_feature (1)..(323): 13601936_protein_ID_13601938
MSLIIVFFFFSLLLTSNGQFFDESKNYEGSSDLVDLQYHLGPVISSPVTSLYIIWYGRWNPTHQSIIRDFLYSVSAPAPAQYPSVSNWWKTVRLYRDQTGSNITDTLVLSGEFHDSTYSHGSHLTRFSVQSVIRTALTSKLPLNAVNGLYLVLTSDDVEMQEFCRAICGFHYFTFPSVVGATVPYAWVGNSERQCPEMCAYPFAQPKPFPGSGFVAREKMKPPNGEVGIDGMISVIAHELAEVSSNPMLNGWYGGEDATAPTEIADLCLGVYGSGGGGGYMGSVYKDRWRNVYNVKGVKGRKYLIQWVWDLNRNRCFGPNAMN

SEQ ID NO: 74 - DNA, 653 bp, Arabidopsis thaliana
  misc_feature (1)..(653): 13604221_construct_ID_YP0110
atcaatcttacatccaaaacttaaagtattcttacatccaaaaacaaaaaaaatatggcaaagtctcttctcatagtaatgctcatgtctatagtaatgttttacatggctcgtccaattttctcccaaaaaattaatccatatttagaggtgatgccaaaagatgtgaccatatctccatcttcaaattttgattacgtcgaagctcccgatgaagctccattcgaagaagctgattcaccagcaatggaatatgacatggagcttgctcaccattattcggacaaacagctcaagtttcttgaggcttgctctgaaaagccgagttcaaaatgcggaaatgaggttttcaagaacatgttaaatgagacgatgctaattacagaggaatgttgtcgtgatatattgaagatgggcaaagattgccatctaggattggttaaactcatatttgccacatatgagtataaaaatattgcatctaagggcattccaaagagcaaacaaacatggaacgaatgtgtccatagagtggggagcaagattggtgctccggtctcttttgaacaatgaactaatatttccgtgtattgatgtgtctatgcgtttttgtaatttgattattactaatataaagcaactgctactatttt

SEQ ID NO: 75 - protein, 172 aa, Arabidopsis thaliana
  misc_feature (1)..(172): 13604221_protein_ID_13604222
MAKSLLIVMLMSIVMFYMARPIFSQKINPYLEVMPKDVTISPSSNFDYVEAPDEAPFEEADSPAMEYDMELAHHYSDKQLKFLEACSEKPSSKCGNEVFKNMLNETMLITEECCRDILKMGKDCHLGLVKLIFATYEYKNIASKGIPKSKQTWNECVHRVGSKIGAPVSFEQ

SEQ ID NO: 76 - DNA, 830 bp, Arabidopsis thaliana
  misc_feature (1)..(830): 13609100_construct_ID_YP0082
acagttctcagataaatactaaactcactgttaaaactttctcaacaaagcttcctgtttctctacaaatggcatctgctctcgctcttaagagactcctatcatcctccatcgctccacgttcccgtagtgttcttcgtccagctgtttcctctcgcctcttcaacaccaacgccgttaggagctacgacgacgacggcgaaaatggagacggcgttgatttatatcgccgctctgttcctcgccgccgtggtgatttcttctcagatgtgtttgatccgttttcgccgacgaggagcgttagtcaagtgctgaatctgatggaccagttcatggagaatcctctgttatcagctactcgtggcatgggagcttcaggagctcgtcgtggttgggatataaaagagaaagacgatgctctgtacctgagaatcgacatgcctgggctgagcagagaggatgtgaagctggctttggagcaggacactctggtgattagaggagaaggaaaaaacgaggaagatggtggcgaggaaggagagagcggtaatcggagattcacaagcaggattggattaccggataagatttacaagatcgatgagattaaggcggagatgaagaacggagtgttgaaagttgtgatcccgaagatgaaagaacaagagagaaatgatgttcgtcagatcgagatcaactaaaaacgtcgacgtttttttctgttctagttttgttgataggtctttgaataagaagtgtgtgtagtttggcacggtcgatgttgagtcatgtagtctctaaagactaaaaggttatatgtttctttcttg

SEQ ID NO: 77 - protein, 210 aa, Arabidopsis thaliana
  misc_feature (1)..(210): 13609100_protein_ID_13609102
MASALALKRLLSSSIAPRSRSVLRPAVSSRLFNTNAVRSYDDDGENGDGVDLYRRSVPRRRGDFFSDVFDPFSPTRSVSQVLNLMDQFMENPLLSATRGMGASGARRGWDIKEKDDALYLRIDMPGLSREDVKLALEQDTLVIRGEGKNEEDGGEEGESGNRRFTSRIGLPDKIYKIDEIKAEMKNGVLKVVIPKMKEQERNDVRQIEIN

SEQ ID NO: 78 - DNA, 995 bp, Arabidopsis thaliana
  misc_feature (1)..(995): 13609583_construct_ID_Bin1-344414-HY2
atttttaacgctcactggatttataagtagagattttttgtgtctcacaaaaacaaaaaaatcatcgtgaaacgttcgaaggccattttctttggacgaccatcggcgttaaggagagagcttagatctcgtgccgtcgtgcgacgttgttttccggcttgatcaaaatggggttgtcattcggaaagttgttcagcaggctctttgcgaagaaagagatgcgtattctgatggttggtctcgatgctgctggtaagacgactatcctctacaagctcaaacttggagagatcgtcaccactattccaaccattgggttcaacgttgagactgttgaatacaagaacatcagcttcaccgtgtgggatgttgggggtcaagacaagatccgtccattgtggagacattacttccagaacacacagggacttatctttgttgtggacagcaatgatcgtgaccgtgttgttgaagccagggacgagcttcacaggatgctgaatgaggatgaattgagggatgcagttctgcttgtatttgctaacaagcaagatcttcccaacgcgatgaacgctgctgagataactgacaagcttgggcttcattctcttcgtcaacgacactggtacattcagagcacatgtgccacctctggagaaggactctatgagggacttgactggctctccaacaacatcgcaagcaaggcatagatggaatgttagccagattcctcttctgcttgtttggtttacaaatcaaagacagaggtctgtttctctagtactaaaagatttattattatattcttcttcgtcacttatctcaaacgcagatcattttacactttgtacttccccttcaataacttgttacttctctcgtttgcttcctgaatttgagtatatcatttttacatctgcttttcatcaaagcataaagcatctttcgaaacaaaaattgaaccgaatttttctgtaaactgatcaaatgtg

SEQ ID NO: 79 - protein, 181 aa, Arabidopsis thaliana
  misc_feature (1)..(181): 13609583_protein_ID_13609584
MGLSFGKLFSRLFAKKEMRILMVGLDAAGKTTILYKLKLGEIVTTIPTIGFNVETVEYKNISFTVWDVGGQDKIRPLWRHYFQNTQGLIFVVDSNDRDRVVEARDELHRMLNEDELRDAVLLVFANKQDLPNAMNAAEITDKLGLHSLRQRHWYIQSTCATSGEGLYEGLDWLSNNIASKA

SEQ ID NO: 80 - DNA, 1761 bp, Arabidopsis thaliana
  misc_feature (1)..(1761): 13609817_construct_ID_YP0094
gcagcagcaaatactatcatcacccatctccttagttctattttataattcctcttctttttgttcatagctttgtaattatagtcttatttctctttaaggctcaataagaggagatgggtgaaaccgctgccgccaataaccaccgtcaccaccaccatcacggccaccaggtctttgacgtggccagccacgatttcgtccctccacaaccggcttttaaatgcttcgatgatgatggccgcctcaaaagaactgggactgtttggaccgcgagcgctcatataataactgcggttatcggatccggcgttttgtcattggcgtgggcgattgcacagctcggatggatcgctggccctgctgtgatgctattgttctctcttgttactctttactcctccacacttcttagcgactgctacagaaccggcgatgcagtgtctggcaagagaaactacacttacatggatgccgttcgatcaattctcggtgggttcaagttcaagatttgtgggttgattcaatacttgaatctctttggtatcgcaattggatacacgatagcagcttccataagcatgatggcgatcaagagatccaactgcttccacaagagtggaggaaaagacccatgtcacatgtccagtaatccttacatgatcgtatttggtgtggcagagatcttgctctctcaggttcctgatttcgatcagatttggtggatctccattgttgcagctgttatgtccttcacttactctgccattggtctagctcttggaatcgttcaagttgcagcgaatggagttttcaaaggaagtctcactggaataagcatcggaacagtgactcaaacacagaagatatggagaaccttccaagcacttggagacattgcctttgcgtactcatactctgttgtcctaatcgagattcaggatactgtaagatccccaccggcggaatcgaaaacgatgaagaaagcaacaaaaatcagtattgccgtcacaactatcttctacatgctatgtggctcaatgggttatgccgcttttggagatgcagcaccgggaaacctcctcaccggttttggattctacaacccgttttggctccttgacatagctaacgccgccattgttgtccacctcgttggagcttaccaagtctttgctcagcccatctttgcctttattgaaaaatcagtcgcagagagatatccagacaatgacttcctcagcaaggaatttgaaatcagaatccccggatttaagtctccttacaaagtaaacgttttcaggatggtttacaggagtggctttgtcgttacaaccaccgtgatatcgatgctgatgccgttttttaacgacgtggtcgggatcttaggggcgttagggttttggcccttgacggtttattttccggtggagatgtatattaagcagaggaaggttgagaaatggagcacgagatgggtgtgtttacagatgcttagtgttgcttgtcttgtgatctcggtggtcgccggggttggatcaatcgccggagtgatgcttgatcttaaggtctataagccattcaagtctacatattgatgattatggaccatgaacaacagagagagttggtgtgtaaagtttaccatttcaaagaaaactccaaaaatgtgtatattgtatgttgttctcatttcgtatggtctcatctttgtaataaaatttaaaacttatgttataaattataaaaccgtgtgttttc

SEQ ID NO: 81 - protein, 493 aa, Arabidopsis thaliana
  misc_feature (1)..(493): 13609817_protein_ID_13609818
MGETAAANNHRHHHHHGHQVFDVASHDFVPPQPAFKCFDDDGRLKRTGTVWTASAHIITAVIGSGVLSLAWAIAQLGWIAGPAVMLLFSLVTLYSSTLLSDCYRTGDAVSGKRNYTYMDAVRSILGGFKFKICGLIQYLNLFGIAIGYTIAASISMMAIKRSNCFHKSGGKDPCHMSSNPYMIVFGVAEILLSQVPDFDQIWWISIVAAVMSFTYSAIGLALGIVQVAANGVFKGSLTGISIGTVTQTQKIWRTFQALGDIAFAYSYSVVLIEIQDTVRSPPAESKTMKKATKISIAVTTIFYMLCGSMGYAAFGDAAPGNLLTGFGFYNPFWLLDIANAAIVVHLVGAYQVFAQPIFAFIEKSVAERYPDNDFLSKEFEIRIPGFKSPYKVNVFRMVYRSGFVVTTTVISMLMPFFNDVVGILGALGFWPLTVYFPVEMYIKQRKVEKWSTRWVCLQMLSVACLVISVVAGVGSIAGVMLDLKVYKPFKSTY

SEQ ID NO: 82 - DNA, 1607 bp, Arabidopsis thaliana
  misc_feature (1)..(1607): 13610584_construct_ID_YP0128
ataatccaaacaccaaaaacaaaatggagaaattgctcgtgatctctttgctactactgatctcaacatcagttacaacttcacaatccgtgaccgatccaatagctttcctccgatgtctcgatagacaaccaacggacccaacaagtcctaactccgccgttgcttacatcccaacaaactcttctttcaccactgtcctccgcagccgtatacctaacctccgtttcgacaaacccactactccaaaacccatctccgtggtggctgccgccacgtggacacacatacaagctgctgtaggatgcgcacgtgagctctctctccaagtcaggatcagaagtggtggccacgacttcgaaggactctcttacacttccaccgtccctttctttgttctcgacatgttcggttttaaaaccgtggacgtaaatctcaccgagagaacggcttgggttgattctggtgctaccctcggagagctttactatagaatctctgagaagagcaatgttcttggatttccggcgggtttgtctaccacattgggcgttggtggacactttagcggcggaggatacggtaatctgatgagaaagtatggtttgtcggtggataacgttttcggctccgggatcgttgattcgaacggaaatatcttcaccgatcgggtttcgatgggggaagaccgtttttgggcgattcgtggaggtggtgcagcgagctacggtgttgtcctcggctacaagatccagctagtaccggtgcctgagaaagttacggtttttaaagtcggaaaaactgtcggagaaggagccgttgatcttataatgaagtggcagagttttgctcatagtacggatcggaatttgttcgtgaggttaactttgactttagtcaacggtacgaagcctggtgagaatacggttttagcgactttcattgggatgtatttaggccggtcggataagctgttgaccgtgatgaaccgggatttcccggagttgaagctgaagaaaaccgattgtaccgagatgagatggatcgattcggttctgttttgggacgattatccggttggtacaccgacttctgtgctactaaatccgctagtcgcaaaaaagttgttcatgaaacgaaaatcggactacgtgaagcgtctgatttcgagaaccgatctcggtttgatactcaagaaattggtagaggttgagaaagttaaaatgaattggaatccgtatggaggaaggatgggtgagatcccgagttcgaggacaccattcccacatagagcaggcaatttgttcaacattgagtatatcatagactggtcagaagctggagataatgtggagaagaaatatttggcactcgcgaatgaattttatagattcatgaccccgtacgtgtctagtaatccgagggaggcgtttttgaattaccgtgatcttgacatagggtcaagtgttaagtctacgtaccaggaaggtaaaatctacggggctaaatatttcaaggagaatttcgagagattagtggatattaaaaccacgattgatgcggaaaacttttggaaaaacgaacaaagcattccggttagaagataa

SEQ ID NO: 83 - protein, 527 aa, Arabidopsis thaliana
  misc_feature (1)..(527): 13610584_protein_ID_13610586
MEKLLVISLLLLISTSVTTSQSVTDPIAFLRCLDRQPTDPTSPNSAVAYIPTNSSFTTVLRSRIPNLRFDKPTTPKPISVVAAATWTHIQAAVGCARELSLQVRIRSGGHDFEGLSYTSTVPFFVLDMFGFKTVDVNLTERTAWVDSGATLGELYYRISEKSNVLGFPAGLSTTLGVGGHFSGGGYGNLMRKYGLSVDNVFGSGIVDSNGNIFTDRVSMGEDRFWAIRGGGAASYGVVLGYKIQLVPVPEKVTVFKVGKTVGEGAVDLIMKWQSFAHSTDRNLFVRLTLTLVNGTKPGENTVLATFIGMYLGRSDKLLTVMNRDFPELKLKKTDCTEMRWIDSVLFWDDYPVGTPTSVLLNPLVAKKLFMKRKSDYVKRLISRTDLGLILKKLVEVEKVKMNWNPYGGRMGEIPSSRTPFPHRAGNLFNIEYIIDWSEAGDNVEKKYLALANEFYRFMTPYVSSNPREAFLNYRDLDIGSSVKSTYQEGKIYGAKYFKENFERLVDIKTTIDAENFWKNEQSIPVRR

SEQ ID NO: 84 - DNA, 1706 bp, Arabidopsis thaliana
  misc_feature (1)..(1706): 13612879_construct_ID_YP0104
gtatctatactcataaatccttttgtctaaaaatggcgatgctaggtttttacgtaacgttcattttctttcttgtatgcctatttacttatttcttcctccaaaagaaacctcaaggtcagcctattctcaagaactggccgttcctcaggatgcttccaggaatgctccaccaaatccctcgtatctacgactggaccgtcgaggtgcttgaggcgaccaatctaactttttatttcaaagggccatggcttagtggaacggacatgttgttcaccgccgatccaaggaatattcatcacatactaagctcaaactttgggaattaccctaaaggacctgagttcaagaagatctttgatgttttgggagaaggaatcttaaccgttgattttgagttgtgggaggagatgaggaagtcaaatcacgccctattccacaatcaagatttcatcgagctctcagtaagtagcaataaaagtaagttaaaagaaggtcttgttccttttcttgataatgctgctcagaaaaacattatcatagaattacaagatgtgttccagagattcatgtttgatacttcttcaattttgatgactggttacgatccaatgtcactatccatcgaaatgctggaagttgagttcggtgaagctgcggatattggcgaagaagcaatctattatagacatttcaaaccggtgatcttgtggaggcttcaaaactggattggtattgggcttgagaggaagatgagaacagctttggccactgtcaatcgtatgtttgcgaagatcatatcttcaagaagaaaagaggagataagtcgcgccaaaacggagccatattccaaggacgcgttgacgtattatatgaatgtggacacgagcaaatataagctcttgaaacctaataaagataagtttataagagatgttatttttagtctagtgttagcaggaagggacaccacaagctcagttctcacttggttcttttggcttctttctaagcatcctcaagttatggccaagctcagacatgagatcaacacaaagtttgataatgaagatctagagaagctcgtgtatctgcatgctgcattgtccgaatcaatgagactctacccgccacttcccttcaaccacaagtctcctgcgaagccagatgtacttccaagcgggcacaaagttgatgcaaattcaaagatcgtgatatgtatctatgcattggggaggatgagatctgtatggggagaagacgcattggatttcaaaccagagagatggatttcagacaatggaggtctaagacatgaaccttcatacaagttcatggcttttaattctggtccgagaacttgcttgggtaaaaatctagctctcttgcagatgaagatggtagctctggagatcatacgaaactatgactttaaggtcattgaaggtcacaaggtcgaaccaattccttctatccttctccgtatgaaacatggtcttaaagtcacagtcacaaagaagatatgattattatgcttgcttggcttctacggcaactattactatttccttatttaaatgtgttacttactagtttgttcccacgttataactacttgtattacgtactaagtacggtgtttgtcccacgtcatgctcataaattaattaatatcgtcaataaagtattagagcatcctcgtccat

SEQ ID NO: 85 - protein, 497 aa, Arabidopsis thaliana
  misc_feature (1)..(497): 13612879_protein_ID_13612881
MAMLGFYVTFIFFLVCLFTYFFLQKKPQGQPILKNWPFLRMLPGMLHQIPRIYDWTVEVLEATNLTFYFKGPWLSGTDMLFTADPRNIHHILSSNFGNYPKGPEFKKIFDVLGEGILTVDFELWEEMRKSNHALFHNQDFIELSVSSNKSKLKEGLVPFLDNAAQKNIIIELQDVFQRFMFDTSSILMTGYDPMSLSIEMLEVEFGEAADIGEEAIYYRHFKPVILWRLQNWIGIGLERKMRTALATVNRMFAKIISSRRKEEISRAKTEPYSKDALTYYMNVDTSKYKLLKPNKDKFIRDVIFSLVLAGRDTTSSVLTWFFWLLSKHPQVMAKLRHEINTKFDNEDLEKLVYLHAALSESMRLYPPLPFNHKSPAKPDVLPSGHKVDANSKIVICIYALGRMRSVWGEDALDFKPERWISDNGGLRHEPSYKFMAFNSGPRTCLGKNLALLQMKMVALEIIRNYDFKVIEGHKVEPIPSILLRMKHGLKVTVTKKI

SEQ ID NO: 86 - DNA, 821 bp, Arabidopsis thaliana
  misc_feature (1)..(821): 13612919_construct_ID_YP0075
aaaaaaagaaccgttttttctttctatggctccaaaactctgagacagagcaagaaaaagataaagtgagtgaaaaaatggcaacggtcacgattctctcacccaaatcgattccaaaggtcactgattccaaattcggagctagggtttctgatcagatcgtcaatgtcgtaaaatgcggcaaatccggccggagattgaagttagcgaagctggtctcagcggctggattgtcacagatcgaaccagacatcaacgaagacccgattggtcaattcgagactaatagcattgaaatggaagatttcaagtatggatattacgatggagctcatacttactatgaaggagaagttcaaaagggaacattttggggagcaattgctgatgacattgctgctgtggatcaaactaatgggtttcaaggtttgatctcttgtatgtttcttcctgctatagctcttgggatgtattttgatgctccgggtgagtacttgttcataggtgcagcgttattcacggtagtgttctgtataatagagatggataaacctgaccagccacacaacttcgagcctcagatatacaaattggagagaggagctcgtgacaagctcattaatgactacaacacaatgagcatttgggactttaatgacaaatatggtgatgtatgggatttcaccattgagaaagatgatatcgccacacgataagataatggattgtgatctcgttataatcatgacttttgatgtaaactgttttataaaattgatgaatgaacggggtacaatgtgtataatattgattgttcattc

SEQ ID NO: 87 - protein, 212 aa, Arabidopsis thaliana
  misc_feature (1)..(212): 13612919_protein_ID_13612921
MATVTILSPKSIPKVTDSKFGARVSDQIVNVVKCGKSGRRLKLAKLVSAAGLSQIEPDINEDPIGQFETNSIEMEDFKYGYYDGAHTYYEGEVQKGTFWGAIADDIAAVDQTNGFQGLISCMFLPAIALGMYFDAPGEYLFIGAALFTVVFCIIEMDKPDQPHNFEPQIYKLERGARDKLINDYNTMSIWDFNDKYGDVWDFTIEKDDIATR

SEQ ID NO: 88 - DNA, 1383 bp, Arabidopsis thaliana
  misc_feature (1)..(1383): 13613553_construct_ID_YP0060
aaacctttctcttctctgctaacgagaaaacaaaagctatcgtctttgctactactactactactattattacattgaatcctttgtgttcttcttcttcagctgctactttgttcgagtgctttcttacatgccgtcggagattgttgacaggaaaaggaagtctcgtggaacacgagatgtagctgagattctaaggcaatggagagagtacaatgagcagattgaggcagaatcttgtatcgatggtggtggtccaaaatcaatccgaaagcctcctccaaaaggttcgaggaagggttgtatgaaaggtaaaggtggacctgaaaacgggatttgtgactatagaggagttagacagaggagatggggtaaatgggttgctgagatccgtgagccagacggaggtgctaggttgtggctcggtactttctccagttcatatgaagctgcattggcttatgacgaggcggccaaagctatatatggtcagtctgccagactcaatcttcccgagatcacaaatcgctcttcttcgactgctgccactgccactgtgtcaggctcggttactgcattttctgatgaatctgaagtttgtgcacgtgaggatacaaatgcaagttcaggttttggtcaggtgaaactagaggattgtagcgatgaatatgttctcttagatagttctcagtgtattaaagaggagctgaaaggaaaagaggaagtgagggaagaacataacttggctgttggttttggaattggacaggactcgaaaagggagactttggatgcttggttgatgggaaatggcaatgaacaagaaccattggagtttggtgtggatgaaacgtttgatattaatgagctattgggtatattaaacgacaacaatgtgtctggtcaagagacaatgcagtatcaagtggatagacacccaaatttcagttaccaaacgcagtttccaaattctaacttgctcgggagcctcaaccctatggagattgctcaaccaggagttgattatggatgtccttatgtgcagcccagtgatatggagaactatggtattgatttagaccatcgcaggttcaatgatcttgacatacaggacttggattttggaggagacaaagatgttcatggatctacataagatttcaaatttcgtttgactggcctaagtttgtgattctgctccgagacggtgtagctgttactagctagaagctgcccttctttgaagctactgatactttctgatattaatggttgtgagacgtagtacatgtagttaggtaatgtaggacaagttcaaatatgattccttctttctttttcttgtgaatacatatgacatatgaagaagttcaaacgttgggt

SEQ ID NO: 89 - protein, 341 aa, Arabidopsis thaliana
  misc_feature (1)..(341): 13613553_protein_ID_13613554
MPSEIVDRKRKSRGTRDVAEILRQWREYNEQIEAESCIDGGGPKSIRKPPPKGSRKGCMKGKGGPENGICDYRGVRQRRWGKWVAEIREPDGGARLWLGTFSSSYEAALAYDEAAKAIYGQSARLNLPEITNRSSSTAATATVSGSVTAFSDESEVCAREDTNASSGFGQVKLEDCSDEYVLLDSSQCIKEELKGKEEVREEHNLAVGFGIGQDSKRETLDAWLMGNGNEQEPLEFGVDETFDINELLGILNDNNVSGQETMQYQVDRHPNFSYQTQFPNSNLLGSLNPMEIAQPGVDYGCPYVQPSDMENYGIDLDHRRFNDLDIQDLDFGGDKDVHGST

SEQ ID NO: 90 - DNA, 1124 bp, Arabidopsis thaliana
  misc_feature (1)..(1124): 13613954_construct_ID_YP0102
aatcacacaaatcccttttttggtttctccaaatcttcaaatcttcttcaatcatcaccatggtacgttttagtaacagtcttgtaggaatactcaacttcttcgtcttccttctctcggttcccatactctcaaccggaatctggctcagccttaaagccacgacgcaatgcgagagattcctcgacaaacccatgatcgctctcggtgttttcctcatgataatcgcaatcgctggagtcgttggatcttgttgcagagtgacgtggcttctctggtcctatctctttgtgatgttcttcttaatcctcatcgtcctctgtttcaccatctttgccttcgttgtcactagtaaaggctccggcgaaactatccaaggaaaagcttataaggagtataggctcgaggcttactctgattggttgcagaggcgtgtgaacaacgctaagcattggaacagcattagaagctgtctttatgagagcaagttctgttataacttggagttagtcactgctaatcacactgtttctgatttctacaaagaagatctcactgcttttgagtctggttgctgcaagccctctaatgactgtgacttcacctacataacttcaacaacttggaataaaacatcaggaacacataaaaactcagattgccaactttgggacaacgaaaagcataagctttgctacaattgcaaagcctgcaaggccggttttctcgacaacctcaaggccgcatggaaaagagttgctattgtcaacatcattttccttgtactcctcgttgtcgtctacgctatgggatgttgcgctttccgaaacaacaaagaagatagatatggccgttccaatggtttcaacaattcttgatttgcgccggttcaagctagactttgatttttcattaatacatcatattacatttatgattagaacaaaacagctttcaaaatttaagaaacagtagaatggaagaatattgaattagtatagttgttgatgtgtttggatttcttctgttgatttgtgtttggacaacagaggattcttcagatctttattacagattgttgtgtttgaagaatcttctatatgaatcttcacttctgacttctg

SEQ ID NO: 91 - protein, 272 aa, Arabidopsis thaliana
  misc_feature (1)..(272): 13613954_protein_ID_13613956
MVRFSNSLVGILNFFVFLLSVPILSTGIWLSLKATTQCERFLDKPMIALGVFLMIIAIAGVVGSCCRVTWLLWSYLFVMFFLILIVLCFTIFAFVVTSKGSGETIQGKAYKEYRLEAYSDWLQRRVNNAKHWNSIRSCLYESKFCYNLELVTANHTVSDFYKEDLTAFESGCCKPSNDCDFTYITSTTWNKTSGTHKNSDCQLWDNEKHKLCYNCKACKAGFLDNLKAAWKRVAIVNIIFLVLLVVVYAMGCCAFRNNKEDRYGRSNGFNNS

SEQ ID NO: 92 - DNA, 987 bp, Arabidopsis thaliana
  misc_feature (1)..(987): 13617784_construct_ID_YP0127
gaaacttgttttctctttcccttcttcaatcaaaacctatttgcatgctctcaaacccgaattaaatcgacacttttcagtttttgttttaacaagtagagtttcccaaaatattggatatatttctttttcaaatttcggaaaagaaatgagttgcaatggatgtagagttcttcgaaaaggttgcagtgaaacatgcatccttcgtccttgccttcaatggatcgaatccgccgagtcacaaggccacgccaccgtcttcgtcgctaaattctttggtcgtgctggtctcatgtctttcatctcctccgtacctgaactccaacgtcctgctttgtttcagtcgttgttgtttgaagcgtgtgggagaacggtgaatccggttaacggagcggttggtatgttgtggaccaggaactggcacgtatgccaagcggcggttgagactgttcttcgcggcggaactttacgaccgatatcagatcttcttgaatctccgtcgttgatgatctcctgtgatgagtcttcagagatttggcatcaagacgtttcaagaaaccaaacccaccattgtcgcttctccacctccagatccacgacggagatgaaagactctctggttaaccgaaaacgattgaagtccgattcggatcttgatctccaagtgaaccacggtttaaccctaaccgctccggctgtaccggttccttttcttcctccgtcgtcgttttgtaaggtggttaagggtgatcgtccgggaagtccatcggaggaatctgtaacgacgtcgtgttgggaaaatgggatgagaggagataataaacaaaaaagaaacaaaggagagaaaaagttattgaacctttttgtttaaaaccgacgacgcaaaacactcaaagattttgaggctctcttttttagggttttgagtgggaatggatatttagttaatgatttttctctatcgagaaatatgataaaattttgggg

SEQ ID NO: 93 - protein, 240 aa, Arabidopsis thaliana
  misc_feature (1)..(240): 13617784_protein_ID_13617786
MSCNGCRVLRKGCSETCILRPCLQWIESAESQGHATVFVAKFFGRAGLMSFISSVPELQRPALFQSLLFEACGRTVNPVNGAVGMLWTRNWHVCQAAVETVLRGGTLRPISDLLESPSLMISCDESSEIWHQDVSRNQTHHCRFSTSRSTTEMKDSLVNRKRLKSDSDLDLQVNHGLTLTAPAVPVPFLPPSSFCKVVKGDRPGSPSEESVTTSCWENGMRGDNKQKRNKGEKKLLNLFV

SEQ ID NO: 94 - DNA, 1673 bp, Arabidopsis thaliana
  misc_feature (1)..(1673): 13647840_construct_ID_YP0186
gaaaaacaaaaaaaagggggaacaagggagtttcatgttaaaaaaaaatgaagctctcttgtttggtttttctcatagtatcgtctcttgtttcgagttctcttgccaccgctccgcccaacacatctatatatgaaagctttctccaatgtttcagcaatcaaacaggtgctcctcctgagaagttatgcgacgtcgttctgcctcaaagcagtgccagcttcactccaaccctacgtgcctacatccgtaacgctcgtttcaacacttccacgtcccccaaacctctgctcgttatcgcggcgcgttctgagtgccacgtccaggccaccgtcctctgcaccaaatctctcaacttccagctcaagactcgcagcggcggccatgactacgacggcgtttcctacatctctaaccgccctttcttcgtcctcgacatgtcctatctccgtaacattaccgtcgatatgtccgacgacggcggctctgcttgggttggagccggcgctactctcggcgaagtttattacaacatttggcagagcagcaaaactcacggcactcacggatttcccgccggtgtttgtcccacagtaggcgctggaggtcacattagcggcgggggctacggcaacatgatcagaaaatacggactttccgtggactacgtcacggacgccaaaatcgtagacgtgaacggacggattctcgatcgtaaatcgatgggagaggatttgttttgggcgattggaggcggtggtggtgcgagcttcggcgtgatcttatctttcaagatcaaactcgtgcctgttcctccgagggtgactgttttcagagtggagaagaccctagtagaaaacgcacttgacatggtccataaatggcagtttgttgctcccaagaccagcccggatctcttcatgaggctaatgttgcagccagtgacccggaacacgactcagacggttcgcgcgtcggtagttgctctgttcttgggaaaacagagcgatctcatgtctctgctgaccaaggagttccccgagcttggtctgaagccggagaattgcacggagatgacgtggatacagtcggtgatgtggtgggccaacaacgacaacgccacggtgattaaaccggagatcctgctggatcgaaatccggattcggcgtctttcttgaaaagaaaatcggattacgtggagaaagagatcagcaaagacggtttagatttcttgtgtaagaagttgatggaggctgggaagctagggctagtgttcaatccatacggagggaaaatgagcgaagttgctacgacggcgactccgttcccacacaggaagaggcttttcaaggtccagcattcgatgaactggaaagacccgggcactgatgttgaaagcagtttcatggaaaagacgagaagcttctacagctacatggctcctttcgtgaccaagaatccaagacacacgtatctcaactacagggatcttgatatcgggatcaacagccatggcccaaacagttacagagaagctgaggtttacgggagaaagtatttcggagagaattttgatcggttggtcaaagtcaaaacagccgtggatccagaaaactttttcagagatgaacaaagtatacctaccttgcctaccaagccatcctcgagttag

SEQ ID NO: 95 - protein, 541 aa, Arabidopsis thaliana
  misc_feature (1)..(541): 13647840_ protein_ID_13647841
MKLSCLVFLIVSSLVSSSLATAPPNTSIYESFLQCFSNQTGAPPEKLCDVVLPQSSASFTPTLRAYIRNARFNTSTSPKPLLVIAARSECHVQATVLCTKSLNFQLKTRSGGHDYDGVSYISNRPFFVLDMSYLRNITVDMSDDGGSAWVGAGATLGEVYYNIWQSSKTHGTHGFPAGVCPTVGAGGHISGGGYGNMIRKYGLSVDYVTDAKIVDVNGRILDRKSMGEDLFWAIGGGGGASFGVILSFKIKLVPVPPRVTVFRVEKTLVENALDMVHKWQFVAPKTSPDLFMRLMLQPVTRNTTQTVRASVVALFLGKQSDLMSLLTKEFPELGLKPENCTEMTWIQSVMWWANNDNATVIKPEILLDRNPDSASFLKRKSDYVEKEISKDGLDFLCKKLMEAGKLGLVFNPYGGKMSEVATTATPFPHRKRLFKVQHSMNWKDPGTDVESSFMEKTRSFYSYMAPFVTKNPRHTYLNYRDLDIGINSHGPNSYREAEVYGRKYFGENFDRLVKVKTAVDPENFFRDEQSIPTLPTKPSSS

SEQ ID NO: 96 - DNA, 1219 bp, Arabidopsis thaliana
  misc_feature (1)..(1219): 13614559_construct_ID_YP0024
gatcaagaaaactcgtctcctacaaaaatcccagaagacaagagattggttcttcttttgcatcattcttacaaaatccccaaaatcattcgaaacccctgagtattctccttaactctaagaaataaatttctgaatggatgcatcgtcttcaccgtctccttccgaggaaagcttgaagcttgagcttgatgatcttcagaaacagctgaacaaaaagctgagattcgaagcatccgtttgttctattcataatcttctccgtgatcactactcttcttcctctccttctctccgcaaacagttctatatagttgtatctcgtgtcgctacggttcttaagacaagatatacagctactggattttgggttgctggactgagtcttttcgaagaggctgagcgacttgtctctgatgcttctgagaagaaacatttgaaatcttgcgttgctcaagctaaggagcagttaagcgaagtagataatcagccaacagagagctcacaaggttatctttttgagggacatcttacggttgatcgtgagccgccacagcctcagtggctagtacagcagaatctcatgtctgctttcgcttctatcgttggtggtgaatcctctaatggtcctactgaaaacactattggggaaactgctaacttgatgcaagaacttatcaatggtcttgacatgatcattccagatatactagatgatggtggaccaccaagagctccaccggcaagtaaagaagttgtagagaaactcccagtcattattttcaccgaggaattgcttaaaaagtttggagcagaggcagaatgttgcatctgcaaggagaatctagttattggcgacaagatgcaggaattgccatgcaagcacacatttcaccctccttgcctaaagccttggctggacgagcataactcttgccctatatgccgccatgaattaccaacagacgatcagaaatacgaaaactggaaagagagagagaaagaggccgaagaagagaggaagggcgcagagaatgctgtccgcggaggtgaatatatgtacgtttaaatttcaatcagttatggcacactcccattgtctttccttgaaacatctccgaattgttgttcatcattcacaattataaatcccattttacatatagattcaatgtcttttgtatgaaagcttataataacaacacagacttctttactt

SEQ ID NO: 97 - protein, 310 aa, Arabidopsis thaliana
  misc_feature (1)..(310): 13614559_protein_ID_13614560
MDASSSPSPSEESLKLELDDLQKQLNKKLRFEASVCSIHNLLRDHYSSSSPSLRKQFYIVVSRVATVLKTRYTATGFWVAGLSLFEEAERLVSDASEKKHLKSCVAQAKEQLSEVDNQPTESSQGYLFEGHLTVDREPPQPQWLVQQNLMSAFASIVGGESSNGPTENTIGETANLMQELINGLDMIIPDILDDGGPPRAPPASKEVVEKLPVIIFTEELLKKFGAEAECCICKENLVIGDKMQELPCKHTFHPPCLKPWLDEHNSCPICRHELPTDDQKYENWKEREKEAEEERKGAENAVRGGEYMYV

SEQ ID NO: 98 - DNA, 1513 bp, Arabidopsis thaliana
  misc_feature (1)..(1513): 13614841_construct_ID_CR13(GFP-ER)
ttcgtactactactaccaccacatttctttagctcaaccttcattactaatctccttttaaggtttctttcgtgaatcagatcggaaaaatggaatcttttttgttcacatctgaatccgtcaacgagggacatcccgacaagctttgtgatcagatctccgacgctatcctcgatgcttgccttgaacaagaccctgagagcaaagttgcttgtgagacttgtaccaagactaacatggtcatggtttttggagaaatcaccaccaaggctaacgttgattacgagcagattgttcgtaaaacatgccgtgagattggattcgtctctgctgacgttggtctagatgctgacaattgcaaggttctggttaacattgagcaacagagtcctgacattgcacaaggtgttcatggtcatctcaccaagaagccagaggaggttggagctggtgaccaaggtcacatgtttgggtatgctactgatgagactcctgagctcatgcctcttactcacgttctcgctactaagcttggagctaaactcactgaagttcgcaagaatggaacttgcccttggttgaggccagatggtaagactcaagtcactattgagtacatcaacgaaagcggagccatggttcctgtacgtgtccacactgttctcatctcaacacagcatgacgagactgtgactaacgatgagatcgcagctgatcttaaggagcatgtgatcaagccagtgatcccagagaaataccttgatgagaaaaccatcttccatctcaacccatctggtcgttttgttatcggaggtcctcatggagatgcagggcttaccggccgtaagatcatcatcgatacttatggtggttggggtgcacacggaggtggtgctttctctggaaaggacccaaccaaggttgacaggagtggggcttacatcgttaggcaagcagctaagagcattgtagccagtgggctagcgaggcgggtcattgtgcaagtctcgtatgccattggtgtccctgagccattgtctgtgttcgtggacagttatggaacaggaaagataccagacaaggagattcttgagattgtgaaggagagttttgatttcaggccaggtatgatctccattaacttggatctgaagagaggaggtaatggtaggttcttgaagactgctgcctatggtcactttggaagggacgatgctgatttcacctgggaggtagtcaagccactcaagtctaacaaggtccaagcttgaaacctgtcagcctctgtttcacttctgtccagaatcagtcttgttctctgtattttaggctctttctgcctctttagtttcaactctgagatgggtttattcattttgttttcaactttgaagaaaaaagctaagcagctgggaatttatataattatttatatggtattcttgtgctaagaaagttaaattcataatatgtatttcttacttattttgagaagaaaatcatataagagaat

SEQ ID NO: 99 - protein, 393 aa, Arabidopsis thaliana
  misc_feature (1)..(393): 13614841_protein_ID_13614842
MESFLFTSESVNEGHPDKLCDQISDAILDACLEQDPESKVACETCTKTNMVMVFGEITTKANVDYEQIVRKTCREIGFVSADVGLDADNCKVLVNIEQQSPDIAQGVHGHLTKKPEEVGAGDQGHMFGYATDETPELMPLTHVLATKLGAKLTEVRKNGTCPWLRPDGKTQVTIEYINESGAMVPVRVHTVLISTQHDETVTNDEIAADLKEHVIKPVIPEKYLDEKTIFHLNPSGRFVIGGPHGDAGLTGRKIIIDTYGGWGAHGGGAFSGKDPTKVDRSGAYIVRQAAKSIVASGLARRVIVQVSYAIGVPEPLSVFVDSYGTGKIPDKEILEIVKESFDFRPGMISINLDLKRGGNGRFLKTAAYGHFGRDDADFTWEVVKPLKSNKVQA

SEQ ID NO: 100 - DNA, 828 bp, Arabidopsis thaliana
  misc_feature (1)..(828): 13617054_construct_ID_YP0117
actcaacacaaactctttacgaatacttttaagtatggcttcttcttctgcaaccaagtttgttgatctgttcccatgtcttttcttagcttgcctcttcgtgttcacatactcaaacaacctcgtcgtggctgaaaattccaacaaagtgaagatcaatctttactatgaatcactttgtccctattgtcaaaatttcattgttgatgatctaggtaaaatctttgactccgatctcctcaaaatcaccgatctcaagctcgttccattcggtaacgctcatatctccaataatctgactattacttgccagcatggtgaagaggaatgcaaacttaacgctctcgaagcttgcggtataagaactttgcccgatccgaaattgcagtacaagttcatacgctgcgttgaaaaagatacgaatgaatgggaatcatgtgttaaaaaatctggacgtgagaaagccatcaatgattgttacaatggtgatctctctcaaaagctgatacttgggtatgcaaaactgacctcgagtttgaagccaaaacatgaatacgtaccatgggtcacactcaacggcaaaccactctatgacaattaccataatttggtcgcacaagtctgcaaagcgtacaaaggaaaggatctcccaaaactatgcagttcctcggtcttgtatgagaggaaagtgtcaaagtttcaagtctcctatgtagatgaagctatcaattaataagttaattaacaaacttcttattgaaactaagatggatctaatctttatgctataagtggaatgataaataaagacgttttatctgaactttt

SEQ ID NO: 101 - protein, 232 aa, Arabidopsis thaliana
  misc_feature (1)..(232): 13617054_protein_ID_13617056
MASSSATKFVDLFPCLFLACLFVFTYSNNLVVAENSNKVKINLYYESLCPYCQNFIVDDLGKIFDSDLLKITDLKLVPFGNAHISNNLTITCQHGEEECKLNALEACGIRTLPDPKLQYKFIRCVEKDTNEWESCVKKSGREKAINDCYNGDLSQKLILGYAKLTSSLKPKHEYVPWVTLNGKPLYDNYHNLVAQVCKAYKGKDLPKLCSSSVLYERKVSKFQVSYVDEAIN

SEQ ID NO: 102 - DNA, 1130 bp, Arabidopsis thaliana
  misc_feature (1)..(1130): 13619323_construct_ID_YP0111
acaaaatatcataaacatataaacataaacgccaatcgcagcttttgtacttttggcggtttacaatggagaaaggtttgacgatgtcttgtgttttggtggtggttgcattcttagccatggttcatgtctctgtttcagttccgttcgtagtgtttcctgaaatcggaacacaatgttctgatgctccaaatgctaacttcacacagcttctcagtaacctctctagctcacctggcttttgcatagaaattggcgagggaaatccaataggcgcttcatggttaataccacttacacaacaagcggaagtagcgtgtgataaggtgacgcagatggaagagttgagtcaaggatacaacattgttggaagagctcaggggagcttagtggctcgaggcttaatcgagttctgcgaaggtgggcctcctgttcacaactatatatccttggctggtcctcatgctggcaccgccgatcttcttcggtgtaatacttctggcttaatttgtgacatagcaaatgggataggcaaggaaaatccctacagcgactttgttcaagataatcttgctcctagtggttatttcaaaaaccctaaaaatgtgacagggtacctgaaagactgtcagtatctacctaagcttaacaatgagagaccatacgaaagaaacacaacttacaaagaccgtttcgcaagtttacagaacctggtttttgtcctgtttgagaacgatacggttattgttccaaaagagtcatcttggttcgggttttatccggatggtgacttaacacatgttctccctgttcaagagacaaagctctatatagaagattggataggtctgaaagcattggttgttgctggaaaagtgcagtttgtgaatgtaaccggtgaccacttaataatggcggacgaagatctcgtcaaatacgtcgtacctcttctccaggatcaacagtctgccccaccaagactcaaccgcaagaccaaggagcccttgcatccttaaaatgagcaaatagttcaatcgctatactaattcatccaatgtcgaataagctcagtgatgattgtgtgacacaataatccttcttcttatatgaataataaaagcatactatct

SEQ ID NO: 103 - protein, 316 aa, Arabidopsis thaliana
  misc_feature (1)..(316): 13619323_protein_ID_13619324
MEKGLTMSCVLVVVAFLAMVHVSVSVPFVVFPEIGTQCSDAPNANFTQLLSNLSSSPGFCIEIGEGNPIGASWLIPLTQQAEVACDKVTQMEELSQGYNIVGRAQGSLVARGLIEFCEGGPPVHNYISLAGPHAGTADLLRCNTSGLICDIANGIGKENPYSDFVQDNLAPSGYFKNPKNVTGYLKDCQYLPKLNNERPYERNTTYKDRFASLQNLVFVLFENDTVIVPKESSWFGFYPDGDLTHVLPVQETKLYIEDWIGLKALVVAGKVQFVNVTGDHLIMADEDLVKYVVPLLQDQQSAPPRLNRKTKEPLHP

SEQ ID NO: 104 - DNA, 929 bp, Arabidopsis thaliana
  misc_feature (1)..(929): 12370095_construct_ID_YP0120
agcactcaacttaaactcttttagtaacaatggtttcttcttctttaaccaagcttgtgttctttggttgtctcctcctgctcacattcacggacaaccttgtggctggaaaatctggcaaagtgaagctcaatctttactacgaatcactttgtcccggttgtcaggaattcatcgtcgatgacctaggtaaaatctttgactacgatctctacacaatcactgatctcaagctgtttccatttggtaatgccgaactctccgataatctgactgtcacttgccagcatggtgaagaggaatgcaaactaaacgcccttgaagcttgcgcattaagaacttggcccgatcagaaatcacaatactcgttcatacggtgcgtcgaaagcgatacgaaaggctgggaatcatgtgttaaaaactctggacgtgagaaagcaatcaatgattgttacaatggtgatctttctagaaagctgatacttgggtacgcaaccaaaaccaagaatttgaagccgccacatgaatacgtaccatgggtcacactcaacggcaagccactcgatgacagcgtacaaagtacggatgatctcgtagctcaaatctgcaatgcatacaaaggaaagactactctcccaaaagtttgcaattcatccgcctcaatgtctaagtcgcctgagaggaaatggaagcttcaagtctcttatgccaataaagctaccaattattaagttaactatcaaacttcgtattgaactaagatggatttaagctttatgttataagtggaatgatgaataaaggcctgttctaaacttttatggttacgaattgatgtattaaaaaagaacatgaaaaacgcctgaactgaactacaagtattttatatgacgtcttatcgacgaaagtgttatgtaactcggtttatc

SEQ ID NO: 105 - protein, 233 aa, Arabidopsis thaliana
  misc_feature (1)..(233): 12370095_protein_ID_12370096
MVSSSLTKLVFFGCLLLLTFTDNLVAGKSGKVKLNLYYESLCPGCQEFIVDDLGKIFDYDLYTITDLKLFPFGNAELSDNLTVTCQHGEEECKLNALEACALRTWPDQKSQYSFIRCVESDTKGWESCVKNSGREKAINDCYNGDLSRKLILGYATKTKNLKPPHEYVPWVTLNGKPLDDSVQSTDDLVAQICNAYKGKTTLPKVCNSSASMSKSPERKWKLQVSYANKATNY

SEQ ID NO: 106 - DNA, 1244 bp, Arabidopsis thaliana
  misc_feature (1)..(1244): 12385291_construct_ID_YP0261
aaacccaacaacataatttcacatatctctctttctttctcttgaaggaaagacgaagatctccaagtcccaagttgttaacacaagacgtaaacatgggtcatcttgggttcttagttatgattatggtaggagtcatggcttcttctgtgagcggctacggtggcggttggatcaacgctcacgccactttttacggtggtggtgatgcttccggcacaatgggtggtgcttgtggatatggtaatctatatagccaaggctacgggacgagcacggcggctctaagcacagctctcttcaacaatggacttagctgtggttcttgctttgagataagatgtgaaaacgatggtaaatggtgtttacctggctcaatcgttgtaaccgctacaaacttctgcccgccaaataacgcgttagcgaacaataatggcggttggtgtaatcctcctcttgaacactttgaccttgctcagcctgtttttcaacgcattgctcagtacagagctggaatcgtccctgtttcctacagaagggttccttgcaggagaagaggaggaataagattcacgataaacggccactcatacttcaaccttgtgctgatcacaaacgtcggtggtgccggagacgttcactcggcggcgatcaagggttcaagaacagtgtggcaagctatgtcaaggaactgggggcaaaattggcaaagcaactcttacctcaacggtcaagcactttcctttaaggtcaccaccagcgacggccgcacagttgtctccttcaacgccgctcctgccggctggtcttatggccagacttttgccggtggacagttccgttaaaaagggcaagttggttaatctctcttccatttatctaaagtaaactcatttgtgtggttatattggtctcttgaaaaaactcggttattgagagagtgatgcgtcgagggctcggttttgcagaaggccttgatgacgtctaatctttttttggacctctttatttttctttcttgaaactagtttttgttaagaaagaaaaaacaagttatagtagttaatgtattactgatgcagaggtggagttttaactaccacccgctagtagtagttatgagttttttattttaaggtgtgagagagagatggattatcaagatttgtcaattttattatgtttgtttgtaataatacaattctttactccagttaatgaaaattgggggattgatcactttt

SEQ ID NO: 107 - protein, 249 aa, Arabidopsis thaliana
  misc_feature (1)..(249): 12385291_protein_ID_12385293
MGHLGFLVMIMVGVMASSVSGYGGGWINAHATFYGGGDASGTMGGACGYGNLYSQGYGTSTAALSTALFNNGLSCGSCFEIRCENDGKWCLPGSIVVTATNFCPPNNALANNNGGWCNPPLEHFDLAQPVFQRIAQYRAGIVPVSYRRVPCRRRGGIRFTINGHSYFNLVLITNVGGAGDVHSAAIKGSRTVWQAMSRNWGQNWQSNSYLNGQALSFKVTTSDGRTVVSFNAAPAGWSYGQTFAGGQFR

SEQ ID NO: 108 - DNA, 1180 bp, Arabidopsis thaliana
  misc_feature (1)..(1180): 12395532_construct_ID_YP0285
acaaataaatacctttgtttccctcttcttctccttcactcacaacatctcaatttcattctctcttctctctccaatttcacaacaatgggagtcaaaagtttcgttgaaggtgggattgcctctgtaatcgccggttgctctactcaccctctcgatctaatcaaggttcgtcttcagcttcacggtgaagcaccttccaccaccaccgtcactctcctccgtccagctctcgctttccccaattcttctcctgcagctttcctggaaacgacttcttcagtccccaaagtaggaccgatctcactcggaatcaacatagtcaaatcggaaggcgccgccgcgttattctcaggagtctccgctacacttctccgtcagacgttatattccaccaccaggatgggtctatacgaagtgcttaagaacaaatggactgatcctgagtcagggaagttgaatctgagtaggaagatcggtgcagggctagtcgctggtggaatcggagccgccgttggaaatccagctgacgtggcgatggttaggatgcaagctgacgggaggttacctttagcgcaacgtcgtaactacgccggagtaggagacgcaatcaggagcatggttaagggagaaggcgtaacgagcttgtggcgaggctcggcgttgacgattaaccgagcgatgattgtgacggcggctcagctagcgtcttacgatcagttcaaggaagggatattggagaatggtgtgatgaatgatgggctagggactcacgtggtagcgagttttgcggcggggtttgttgcttcggttgcgtctaatccggtggatgtgataaagacgagagtgatgaatatgaaggtgggagcgtacgacggcgcgtgggattgtgcggtgaagacggttaaagcggaaggagccatggctctttataaaggctttgttcctacagtttgtaggcaaggtcctttcactgttgttctcttcgttacgttggagcaagttaggaagctgcttcgagatttttgataccattcttttattgatgatgatgatggcgactatttatattgatttattcatttttgaaatagtgaacacaagaaggaactaggaagagggggattcaatatattttttgttcaagcattgttgttaaatacaattcaattttagtttc

SEQ ID NO: 109 - protein, 313 aa, Arabidopsis thaliana
  misc_feature (1)..(313): 12395532_protein_ID_12395534
MGVKSFVEGGIASVIAGCSTHPLDLIKVRLQLHGEAPSTTTVTLLRPALAFPNSSPAAFLETTSSVPKVGPISLGINIVKSEGAAALFSGVSATLLRQTLYSTTRMGLYEVLKNKWTDPESGKLNLSRKIGAGLVAGGIGAAVGNPADVAMVRMQADGRLPLAQRRNYAGVGDAIRSMVKGEGVTSLWRGSALTINRAMIVTAAQLASYDQFKEGILENGVMNDGLGTHVVASFAAGFVASVASNPVDVIKTRVMNMKVGAYDGAWDCAVKTVKAEGAMALYKGFVPTVCRQGPFTVVLFVTLEQVRKLLRDF

SEQ ID NO: 110 - DNA, 2509 bp, Arabidopsis thaliana
  misc_feature (1)..(2509): 12575820_construct_ID_YP0216
tctctataaatccttatatgttttacttacattcctaaagttttcaactttcttgagcttcaaaaagtacctccaatggcttcttctgcatttgcttttccttcttacataataaccaaaggaggactttcaactgattcttgtaaatcaacttctttgtcttcttctagatctttggttacagatcttccatcaccatgtctgaaacccaacaacaattcccattcaaacagaagagcaaaagtgtgtgcttcacttgcagagaagggtgaatattattcaaacagaccaccaactccattacttgacactattaactacccaatccacatgaaaaatctttctgtcaaggaactgaaacaactttctgatgagctgagatcagacgtgatctttaatgtgtcgaaaaccggtggacatttggggtcaagtcttggtgttgtggagcttactgtggctcttcattacattttcaatactccacaagacaagattctttgggatgttggtcatcagtcttatcctcataagattcttactgggagaagaggaaagatgcctacaatgaggcaaaccaatggtctctctggtttcaccaaacgaggagagagtgaacatgattgctttggtactggacacagctcaaccacaatatctgctggtttaggaatggcggtaggaagggatttgaaggggaagaacaacaatgtggttgctgtgattggtgatggtgcgatgacggcaggacaggcttatgaagccatgaacaacgccggatatctagactctgatatgattgtgattcttaatgacaacaagcaagtctcattacctacagctactttggatggaccaagtccacctgttggtgcattgagcagtgctcttagtcggttacagtctaacccggctctcagagagttgagagaagtcgcaaagggtatgacaaagcaaataggcggaccaatgcatcagttggcggctaaggtagatgagtatgctcgaggaatgataagcgggactggatcgtcactgtttgaagaactcggtctttactatattggtccagttgatgggcacaacatagatgatttggtagccattcttaaagaagttaagagtaccagaaccacaggacctgtacttattcatgtggtgacggagaaaggtcgtggttatccttacgcggagagagctgatgacaaataccatggtgttgtgaaatttgatccagcaacgggtagacagttcaaaactactaataagactcaatcttacacaacttactttgcggaggcattagtcgcagaagcagaggtagacaaagatgtggttgcgattcatgcagccatgggaggtggaaccgggttaaatctctttcaacgtcgcttcccaacaagatgtttcgatgtaggaatagcggaacaacacgcagttacttttgctgcgggtttagcctgtgaaggccttaaacccttctgtgcaatctattcgtctttcatgcagcgtgcttatgaccaggttgtccatgatgttgatttgcaaaaattaccggtgagatttgcaatggatagagctggactcgttggagctgatggtccgacacattgtggagctttcgatgtgacatttatggcttgtcttcctaacatgatagtgatggctccatcagatgaagcagatctctttaacatggttgcaactgctgttgcgattgatgatcgtccttcttgtttccgttaccctagaggtaacggtattggagttgcattacctcccggaaacaaaggtgttccaattgagattgggaaaggtagaattttaaaggaaggagagagagttgcgttgttgggttatggctcagcagttcagagctgtttaggagcggctgtaatgctcgaagaacgcggattaaacgtaactgtagcggatgcacggttttgcaagccattggaccgtgctctcattcgcagcttagctaagtcgcacgaggttctgatcacggttgaagaaggttccattggaggttttggctcgcacgttgttcagtttcttgctctcgatggtcttcttgatggcaaactcaagtggagaccaatggtactgcctgatcgatacattgatcacggtgcaccagctgatcaactagctgaagctggactcatgccatctcacatcgcagcaaccgcacttaacttaatcggtgcaccaagggaagctctgttttgagagtaagaatctgttggctaaaacatatgtatacaaacactctaaatgcaacccaaggtttcttctaagtactgatcagaattcccgccgagaagtcctttggcaacagctatatatatttactaagattgtgaagagaaaggcaaaggcaaaggttgtgcaaagattagtattatgataaaactggtatttgttttgtaattttgtttaggattgtgatggagatcgtgttgtacaataatctaacatcttgtaaaaatcaattacatctctttgtgta

SEQ ID NO: 111 - protein, 717 aa, Arabidopsis thaliana
  misc_feature (1)..(717): 12575820_protein_ID_12575821
MASSAFAFPSYIITKGGLSTDSCKSTSLSSSRSLVTDLPSPCLKPNNNSHSNRRAKVCASLAEKGEYYSNRPPTPLLDTINYPIHMKNLSVKELKQLSDELRSDVIFNVSKTGGHLGSSLGVVELTVALHYIFNTPQDKILWDVGHQSYPHKILTGRRGKMPTMRQTNGLSGFTKRGESEHDCFGTGHSSTTISAGLGMAVGRDLKGKNNNVVAVIGDGAMTAGQAYEAMNNAGYLDSDMIVILNDNKQVSLPTATLDGPSPPVGALSSALSRLQSNPALRELREVAKGMTKQIGGPMHQLAAKVDEYARGMISGTGSSLFEELGLYYIGPVDGHNIDDLVAILKEVKSTRTTGPVLIHVVTEKGRGYPYAERADDKYHGVVKFDPATGRQFKTTNKTQSYTTYFAEALVAEAEVDKDVVAIHAAMGGGTGLNLFQRRFPTRCFDVGIAEQHAVTFAAGLACEGLKPFCAIYSSFMQRAYDQVVHDVDLQKLPVRFAMDRAGLVGADGPTHCGAFDVTFMACLPNMIVMAPSDEADLFNMVATAVAIDDRPSCFRYPRGNGIGVALPPGNKGVPIEIGKGRILKEGERVALLGYGSAVQSCLGAAVMLEERGLNVTVADARFCKPLDRALIRSLAKSHEVLITVEEGSIGGFGSHVVQFLALDGLLDGKLKWRPMVLPDRYIDHGAPADQLAEAGLMPSHIAATALNLIGAPREALF

SEQ ID NO: 112 - DNA, 1544 bp, Arabidopsis thaliana
  misc_feature (1)..(1544): 12600234_construct_ID_YP0279
atgtcggcgtgtttaagcagcggaggaggaggagcagcagcatatagtttcgagttagaaaaagtgaaatcaccaccaccatcatcctcaacaacaacaacaagagctacttcaccatcatcaacaatctccgaatcatcaaattcaccactcgcaatctcaacgagaaagccaagaacacaacgcaaaagaccaaaccagacttacaacgaagcagctactcttctctctactgcttatcccaacatcttctcctcaaacttgtcctctaagcaaaaaactcactcttcatcaaactctcacttctacgggccattgcttagtgacaacgacgacgcttctgatttgcttcttccttatgaatcaatcgaagaacctgattttctgtttcatccaacgattcaaacgaaaacagagtttttctcagaccagaaggaagttaactccggtggagattgctacggtggtgaaatcgaaaagtttgatttctccgacgaattcgatgctgaatcgattctcgatgaggatattgaagaaggaatcgatagtataatggggactgtggtggaatcgaattcaaattcggggatttatgaatctagggttccgggaatgatcaatcgcggtggaagaagttcttctaatcggattggtaaactagaacagatgatgatgatcaattcatggaatcgaagctctaacggattcaatttcccgttagggcttggattacgaagtgctctcagagaaaacgacgacacaaaattgtggaagattcataccgttgatttcgaacagatctcgccgcgaattcaaactgtcaaaaccgaaactgcaatctccaccgttgatgaggagaaatccgacggtaagaaggtggtaatctctggagagaagagtaataagaagaagaagaagaagaaaatgacggtgacgacgacattgattacggaatcgaaaagcttggaagatacggaggagacgagtttgaagagaacaggtccgttgttgaagcttgattacgacggcgttttggaagcttggtctgataaaacgtcgccgtttcccgacgagattcagggatcggaagctgtcgatgtcaatgctagattagctcagattgatttgttcggagacagtggaatgcgagaagcaagtgttttgaggtacaaagagaaacgtcgaactcgtcttttttcgaagaaaattcgataccaagttcgcaaactcaatgctgatcaacgtcctcgaatgaagggacgattcgtgagaaggcccaatgagagcactccaagtggacaaagataacaaggataaaagagcctagatttatcttatctttttttttttatcttttgtttattccttgttttatttttgtttctaaaattttggcaccctccttttttgtttcttttaagttatggtcccttttggtttataatttagattttttgatgagggggagatttgattgagaaagtgagggatcaaaactaataaaagtttttgttattaatagaagaaacagagctcttgagatt

SEQ ID NO: 113 - protein, 435 aa, Arabidopsis thaliana
  misc_feature (1)..(435): 12600234_protein_ID_12600235
MSACLSSGGGGAAAYSFELEKVKSPPPSSSTTTTRATSPSSTISESSNSPLAISTRKPRTQRKRPNQTYNEAATLLSTAYPNIFSSNLSSKQKTHSSSNSHFYGPLLSDNDDASDLLLPYESIEEPDFLFHPTIQTKTEFFSDQKEVNSGGDCYGGEIEKFDFSDEFDAESILDEDIEEGIDSIMGTVVESNSNSGIYESRVPGMINRGGRSSSNRIGKLEQMMMINSWNRSSNGFNFPLGLGLRSALRENDDTKLWKIHTVDFEQISPRIQTVKTETAISTVDEEKSDGKKVVISGEKSNKKKKKKKMTVTTTLITESKSLEDTEETSLKRTGPLLKLDYDGVLEAWSDKTSPFPDEIQGSEAVDVNARLAQIDLFGDSGMREASVLRYKEKRRTRLFSKKIRYQVRKLNADQRPRMKGRFVRRPNESTPSGQR

SEQ ID NO: 114 - DNA, 3008 bp, Arabidopsis thaliana
  misc_feature (1)..(3008): 12603755_construct_ID_YP0080
atttttgtttttatttttctgatgttacaatggcagacaagatcttcactttcttcctaatcttgtcttcgatctctcctctcttatgctcttctttgatctcacctcttaatctctcacttattagacaagcaaatgtccttatctctctaaagcaaagttttgattcctatgatccttctcttgattcatggaacattccaaatttcaactctctatgttcttggactggtgtttcttgtgacaacttgaatcagtctattactcgtctagacctatctaatctcaacatctccggcactatctctccggaaatatctcgtctttcgccgtcacttgtttttcttgacatttcttctaacagtttctccggtgagcttcctaaagagatctatgagctctcaggcctcgaagtgttaaacatctctagcaatgtttttgaaggagagctggagacacgtgggttcagtcaaatgactcagcttgtgactcttgacgcttacgacaacagcttcaacggatcacttcctctgagtctaaccacactcactcgtctcgagcacttagatcttggaggaaactacttcgacggtgagatccctagaagctatggaagtttcttgagtctcaagtttctttctttatctggtaatgatctccgtgggagaatccctaacgagctagcgaacatcacgactttggtacagctttacttaggttactacaacgattaccgcggtgggatacctgcagatttcgggagattgatcaatcttgttcatttggatttagctaattgcagcttgaaaggatcaattcctgcagaattggggaatctcaagaacttggaggttctgtttcttcagaccaatgagcttacaggctctgttcctcgagagttagggaacatgacaagcctcaagactcttgatctctccaacaactttcttgaaggagagattcctctagagctatctggacttcaaaagcttcagttgtttaacctcttcttcaacagactacacggcgagatccctgagttcgtatctgagcttcctgatctgcaaatactcaagctttggcacaacaatttcaccggaaagattccttcgaaactcggatcaaacgggaacttgatcgagatcgatttgtctaccaataaactcacaggtttgatccctgagtcactctgtttcggaagaagactaaagattctcattctcttcaacaacttcttgttcggtcctctccctgaagatcttggccaatgtgaaccgctatggagattccgtctcggacagaactttctgacaagtaagttgccaaagggtttgatttatttgccgaatctttcgcttcttgagcttcaaaacaactttttgactggagaaatccccgaagaagaggcgggaaatgcgcagttttcgagccttactcagatcaatctgtccaacaacaggttatccggaccgattcctggttcaatcagaaacctcagaagccttcagattcttcttctcggtgcaaaccggttatcgggacagatccctggcgaaatcggaagtttgaagagtcttctcaagattgacatgagcagaaacaacttctcaggcaagtttcctcctgagtttggtgattgcatgtcactcacatatttagatttgagtcacaaccagatttccggtcagattccggttcagatatcgcagattcggattctaaactatctgaatgtttcttggaattcctttaaccaaagccttcccaacgaactcggatacatgaagagtttaacatcagcagatttctcacacaacaacttctccggttcagtaccaacttcagggcaattctcttacttcaacaacacgtcattccttggaaacccttttctctgtggattttcttcaaacccttgcaacggttcccaaaaccaatctcaatctcagctacttaaccagaacaacgcaagatcccgaggtgaaatctccgcaaaattcaagttgttcttcgggttaggcctactagggtttttcttggtgttcgtcgttttagctgtggtcaagaataggagaatgagaaagaacaacccgaatttatggaagcttatagggtttcagaagctcggtttcagaagcgaacacatattagaatgtgttaaagagaaccatgtgattgggaaaggcggacgagggattgtctacaaaggggtaatgccaaacggagaagaagttgcagtcaagaagctcttaaccataaccaaaggatcatctcatgacaacggtttagccgcagagattcagacattaggtagaatcagacacagaaacatagtgagattgctcgctttttgttcaaacaaagacgtgaatctccttgtttacgagtatatgcctaatggtagcctcggagaagtcttgcacgggaaagctggagtgtttttgaaatgggaaacacggttgcaaatagcgttggaagcggctaaggggttgtgttatcttcaccatgattgctcgccacttataatccaccgtgatgtgaagtcaaacaacatcttgttgggtcctgagtttgaagctcatgttgctgattttgggcttgctaagtttatgatgcaagacaatggagcttccgagtgcatgtcctcgatcgctggctcgtacggctacatcgctccagaatatgcatatacactgagaatagacgagaagagcgatgtgtacagcttcggagtagtgttattggagctgattacgggtcgaaaaccagtagataattttggggaagaagggatagacattgtgcaatggtcaaagatccaaacaaactgtaacagacaaggtgtggtgaagatcattgaccagagattgagcaatattccattagcagaggccatggaactgttctttgtggcaatgctatgtgtgcaagaacatagtgttgagagaccgaccatgagagaggttgtccagatgatctctcaggctaaacagcctaatactttctaa

SEQ ID NO: 115 - protein, 992 aa, Arabidopsis thaliana
  misc_feature (1)..(992): 12603755_protein_ID_12603757
MADKIFTFFLILSSISPLLCSSLISPLNLSLIRQANVLISLKQSFDSYDPSLDSWNIPNFNSLCSWTGVSCDNLNQSITRLDLSNLNISGTISPEISRLSPSLVFLDISSNSFSGELPKEIYELSGLEVLNISSNVFEGELETRGFSQMTQLVTLDAYDNSFNGSLPLSLTTLTRLEHLDLGGNYFDGEIPRSYGSFLSLKFLSLSGNDLRGRIPNELANITTLVQLYLGYYNDYRGGIPADFGRLINLVHLDLANCSLKGSIPAELGNLKNLEVLFLQTNELTGSVPRELGNMTSLKTLDLSNNFLEGEIPLELSGLQKLQLFNLFFNRLHGEIPEFVSELPDLQILKLWHNNFTGKIPSKLGSNGNLIEIDLSTNKLTGLIPESLCFGRRLKILILFNNFLFGPLPEDLGQCEPLWRFRLGQNFLTSKLPKGLIYLPNLSLLELQNNFLTGEIPEEEAGNAQFSSLTQINLSNNRLSGPIPGSIRNLRSLQILLLGANRLSGQIPGEIGSLKSLLKIDMSRNNFSGKFPPEFGDCMSLTYLDLSHNQISGQIPVQISQIRILNYLNVSWNSFNQSLPNELGYMKSLTSADFSHNNFSGSVPTSGQFSYFNNTSFLGNPFLCGFSSNPCNGSQNQSQSQLLNQNNARSRGEISAKFKLFFGLGLLGFFLVFVVLAVVKNRRMRKNNPNLWKLIGFQKLGFRSEHILECVKENHVIGKGGRGIVYKGVMPNGEEVAVKKLLTITKGSSHDNGLAAEIQTLGRIRHRNIVRLLAFCSNKDVNLLVYEYMPNGSLGEVLHGKAGVFLKWETRLQIALEAAKGLCYLHHDCSPLIIHRDVKSNNILLGPEFEAHVADFGLAKFMMQDNGASECMSSIAGSYGYIAPEYAYTLRIDEKSDVYSFGVVLLELITGRKPVDNFGEEGIDIVQWSKIQTNCNRQGVVKIIDQRLSNIPLAEAMELFFVAMLCVQEHSVERPTMREVVQMISQAKQPNTF

SEQ ID NO: 116 - DNA, 1308 bp, Arabidopsis thaliana
  misc_feature (1)..(1308): 12640578_construct_ID_YP0263
gtcccatcaccaaacattaagtagcactctttttcctctctatatctctcactcacactttttctctatatcttctcctcaacttggatatgggtgaagccgtagaggtcatgttcggaaatgggttcccggagattcacaaagccacatcacccactcaaaccctccactctaaccagcaagactgccattggtatgaagaaaccatcgatgatgatctcaagtggtcttttgccctcaacagtgttctccatcaaggaactagtgagtaccaagatattgctctgttggacaccaaacgttttggaaaggtgcttgtgattgatgggaaaatgcaaagtgctgagagagatgagtttatctaccatgaatgtttgatccatcccgctctccttttccatcccaaccccaagactgtgtttataatgggaggaggtgaaggctctgctgcaagagaaatactaaaacacacgacgatcgagaaagttgttatgtgtgatattgatcaggaagttgttgatttttgcagaagatttctgaccgttaacagcgatgctttctgtaacaaaaagcttgaacttgtgatcaaagatgcaaaggctgaattagagaaaagggaagagaagtttgatatcatagtgggagatttagctgatccagtggaaggtggaccttgttatcagctctacaccaaatccttctaccaaaacattctcaaacccaagcttagccctaatggcatttttgtcacccaggctggaccagcaggaatattcactcataaggaagtcttcacatcaatctacaacaccatgaagcaagtcttcaagtacgtgaaggcttacacagcacatgtgccatcatttgcggacacatggggatgggtgatggcatcggaccacgagtttgacgttgaagttgatgaaatggatcgaagaatcgaagagagagttaacggagaattgatgtatctaaacgctccttctttcgtctctgctgctactctcaacaaaaccatctctctcgcgctagagaaggagactgaagtttatagtgaagagaatgcgagattcattcatggtcatggtgtggcgtaccggcatatttaaagacgaaccggtttcagtttcagtgttattaccaaacccatgtcacaaaaacaaaaggccggtttcttttctccgcacagaaccgggtgttgtcttgaatcttgattactttggttcggttttattttctacattgctttttgttttcttgttcttccctcaagttattccggtttaacaagactatattgcttactaa

SEQ ID NO: 117 - protein, 339 aa, Arabidopsis thaliana
  misc_feature (1)..(339): 12640578_protein_ID_12640579
MGEAVEVMFGNGFPEIHKATSPTQTLHSNQQDCHWYEETIDDDLKWSFALNSVLHQGTSEYQDIALLDTKRFGKVLVIDGKMQSAERDEFIYHECLIHPALLFHPNPKTVFIMGGGEGSAAREILKHTTIEKVVMCDIDQEVVDFCRRFLTVNSDAFCNKKLELVIKDAKAELEKREEKFDIIVGDLADPVEGGPCYQLYTKSFYQNILKPKLSPNGIFVTQAGPAGIFTHKEVFTSIYNTMKQVFKYVKAYTAHVPSFADTWGWVMASDHEFDVEVDEMDRRIEERVNGELMYLNAPSFVSAATLNKTISLALEKETEVYSEENARFIHGHGVAYRHI

SEQ ID NO: 118 - DNA, 1074 bp, Arabidopsis thaliana
  misc_feature (1)..(1074): 12647555_construct_ID_YP0018
atctcacatcacaattcacatctcctcgaacaaacaaattataaacccattttccttcataaatttctaaaataaaaccccttaaactttcattcacatcatccaacccccaatgggtcgaatcttgaaccgtaccgtgttaatgactcttctagtcgtaacaatggccggaacagcattctccggtagcttcaacgaagagtttgacttaacttggggtgaacacagaggcaaaatcttcagtggaggaaaaatgttgtcactctcactagaccgggtttccgggtcgggttttaaatccaagaaagaatatttgttcggaagaatcgacatgcagcttaaactcgtcgccggtaactccgctggaaccgtcactgcctactacttgtcatcggaaggaccaacacacgacgagatagactttgagtttcttggtaatgaaacagggaagccttatgttcttcacactaatgtatttgctcaaggcaaaggaaacagagaacaacagttttatctctggtttgatccaaccaagaacttccacacttattctcttgtctggagaccacaacacatcatatttatggtagataatgttccaatcagagtattcaacaatgcagagcaacttggtgttccatttcccaagaaccaaccaatgaagatatactcgagtttatggaatgcagatgattgggctacaagaggtggtttggttaagacagattggtctaaagctcctttcacagcttactacagaggctttaacgctgcagcttgtactgtttcttcagggtcatctttctgtgatcctaagtttaagagttcttttactaatggtgaatctcaagtggctaatgagcttaatgcttatgggagaagaagattaagatgggttcagaagtattttatgatttatgattattgttctgatttaaaaaggtttcctcaaggattcccaccagagtgtaggaagtctagagtctaaaaaccaatgattctctctttgttgttgtttagtgcaaattaaattctctttgttgtttctttaataaattgatttgatttttcttc

SEQ ID NO: 119 - protein, 291 aa, Arabidopsis thaliana
  misc_feature (1)..(291): 12647555_protein_ID_12647556
MGRILNRTVLMTLLVVTMAGTAFSGSFNEEFDLTWGEHRGKIFSGGKMLSLSLDRVSGSGFKSKKEYLFGRIDMQLKLVAGNSAGTVTAYYLSSEGPTHDEIDFEFLGNETGKPYVLHTNVFAQGKGNREQQFYLWFDPTKNFHTYSLVWRPQHIIFMVDNVPIRVFNNAEQLGVPFPKNQPMKIYSSLWNADDWATRGGLVKTDWSKAPFTAYYRGFNAAACTVSSGSSFCDPKFKSSFTNGESQVANELNAYGRRRLRWVQKYFMIYDYCSDLKRFPQGFPPECRKSRV

SEQ ID NO: 120 - DNA, 1537 bp, Arabidopsis thaliana
  misc_feature (1)..(1537): 12649228_construct_ID_YP0003
gctcctttctcgtctctgtcttcttcgtcctcattcgttttaaagcatcaaaatttcatcaacccaaaatagattaaaaaaatctgtagctttcgcatgtaaatctctctttgaaggttcctaactcgttaatcgtaactcacagtgactcgttcgagtcaaagtctctgtctttagctcaaaccatggctagtaacaaccctcacgacaacctttctgaccaaactccttctgatgatttcttcgagcaaatcctcggccttcctaacttctcagcctcttctgccgccggtttatctggagttgacggaggattaggtggtggagcaccgcctatgatgctgcagttgggttccggagaagaaggaagtcacatgggtggcttaggaggaagtggaccaactgggtttcacaatcagatgtttcctttggggttaagtcttgatcaagggaaaggacctgggtttcttagacctgaaggaggacatggaagtgggaaaagattctcagatgatgttgttgataatcgatgttcttctatgaaacctgttttccacgggcagcctatgcaacagccacctccatcggccccacatcagcctacttcaatccgtcccagggttcgagctaggcgtggtcaggctactgatccacatagcatcgctgagcggctacgtagagaaagaatagcagaacggatcagggcgctgcaggaacttgtacctactgtgaacaagaccgatagagctgctatgatcgatgagattgtcgattatgtaaagtttctcaggctccaagtcaaggttttgagcatgagccgacttggtggagccggtgcggttgctccacttgttactgatatgcctctttcatcatcagttgaggatgaaacgggtgagggtggaaggactccgcaaccagcgtgggagaaatggtctaacgatgggactgaacgtcaagtggctaaactgatggaagagaacgttggagccgcgatgcagcttcttcaatcaaaggctctttgtatgatgccaatctcattggcaatggcaatttaccattctcaacctccggatacatcttcagtggtcaagcctgagaacaatcctccacagtaggatttctgcaataaagagtttgtacagctaatccaactgtccaacatgggtttttcttctgctctaatgactctggtttcttctctcctctctcacccacttgaaaggtaaaaaagtgaaaaaggctttgtagatggaatcaatgtaggatttgcagtagagggaaaaaaaatgtcaaaaagctcaattgatcaagtattattgtaatcattgtacctttattttaggtggactttgatgaaagcaactttttgttttcaagactttagtgggaggttgaggaaggagcttgaagggtgttatttattagtagtagtagtagtgggaagttgtgggaccttgttgagttgtgttcaaattgaagaaaaaacaagtatttgtaatttgtcaccccttgtattattatttattttgtatga

SEQ ID NO: 121 - protein, 310 aa, Arabidopsis thaliana
  misc_feature (1)..(310): 12649228_protein_ID_12649229
MASNNPHDNLSDQTPSDDFFEQILGLPNFSASSAAGLSGVDGGLGGGAPPMMLQLGSGEEGSHMGGLGGSGPTGFHNQMFPLGLSLDQGKGPGFLRPEGGHGSGKRFSDDVVDNRCSSMKPVFHGQPMQQPPPSAPHQPTSIRPRVRARRGQATDPHSIAERLRRERIAERIRALQELVPTVNKTDRAAMIDEIVDYVKFLRLQVKVLSMSRLGGAGAVAPLVTDMPLSSSVEDETGEGGRTPQPAWEKWSNDGTERQVAKLMEENVGAAMQLLQSKALCMMPISLAMAIYHSQPPDTSSVVKPENNPPQ

SEQ ID NO: 122 - DNA, 1273 bp, Arabidopsis thaliana
  misc_feature (1)..(1273): 12658070_construct_ID_YP0271
cacacttaaagctttcgtctttacctcttcccttctctctctctatctaaaaagagttccgagaagaagatcatcatcaatggcgacttctctcttcttcatgtcaacagatcaaaactccgtcggaaacccaaacgatcttctgagaaacacccgtcttgtcgtcaacagctccggcgagatccggacagagacactgaagagtcgtggtcggaaaccaggatcgaagacaggtcagcaaaaacagaagaaaccaacgttgagaggaatgggtgtagcaaagctcgagcgtcagagaatcgaagaagaaaagaagcaactcgccgccgccacagtcggagacacgtcatcagtagcatcgatctctaacaacgctacccgtttacccgtaccggtagacccgggtgttgtgctacaaggcttcccaagctcactcgggagcaacaggatctattgtggtggagtcgggtcgggtcaggttatgatcgacccggttatttctccatggggttttgttgagacctcctccactactcatgagctctcttcaatctcaaatcctcaaatgtttaacgcttcttccaataatcgctgtgacacttgcttcaagaagaaacgtttggatggtgatcagaataatgtagttcgatccaacggtggtggattttcgaaatacacaatgattcctcctccgatgaacggctacgatcagtatcttcttcaatcagatcatcatcagaggagccaaggtttcctttatgatcatagaatcgctagagcagcttcagtttctgcttctagtactactattaatccttatttcaacgaggcaacaaatcatacgggaccaatggaggaatttgggagctacatggaaggaaaccctagaaatggatcaggaggtgtgaaggagtacgagttttttccggggaaatatggtgaaagagtttcagtggtggctaaaacgtcgtcactcgtaggtgattgcagtcctaataccattgatttgtccttgaagctttaaatgttttatctttctatattgatttaaacaaaatcgtctctttaaagaaaaaacattttaagtagatgaaagtaagaaacagaagaaaaaaaagagagagccttttttggtgtatgcatctgagagctgagtcgaaagaaagattcagcttttggattacccttttggttgtttattatgagattctaacctaaacactcagacatatatgttctgttctcttccttaattgttgtcatgaaacttctc

SEQ ID NO: 123 - protein, 314 aa, Arabidopsis thaliana
  misc_feature (1)..(314): 12658070_protein_ID_12658072
MATSLFFMSTDQNSVGNPNDLLRNTRLVVNSSGEIRTETLKSRGRKPGSKTGQQKQKKPTLRGMGVAKLERQRIEEEKKQLAAATVGDTSSVASISNNATRLPVPVDPGVVLQGFPSSLGSNRIYCGGVGSGQVMIDPVISPWGFVETSSTTHELSSISNPQMFNASSNNRCDTCFKKKRLDGDQNNVVRSNGGGFSKYTMIPPPMNGYDQYLLQSDHHQRSQGFLYDHRIARAASVSASSTTINPYFNEATNHTGPMEEFGSYMEGNPRNGSGGVKEYEFFPGKYGERVSVVAKTSSLVGDCSPNTIDLSLKL

SEQ ID NO: 124 - DNA, 519 bp, Arabidopsis thaliana
  misc_feature (1)..(519): 12676237_construct_ID_YP0230
cgaaggcacgacaagcatcaatccgcctcaagcagtagcagcaggaaacgtagcagggaacatggcaggagctcatggaatgggcagtagatcgatgccaagaccaatggttgcacataacatgcagaggatgcagcaatctcaaggcatgatggcttataatttcccggcacaggcagggcttaacccgagtgttccgctgcagcagcagcgcgggatggctcaaccgcaccagcagcaacagctaagaaggaaagatcccggaatgggtatgtcaggttacgcacctcctaacaaatccagacgcctctaaaggtaaaatcgagatcatcagtctcgggttagaatctgtgtgtttgccgcagaagaaagcgttgcgatttgctttatagagtagagttagattgtaatgcagcatgtggaatgttgctattcatatggatggattggattctctgtagtttttgtataaacatcctctcaagtatttgttaattatattagatcatcatttctctt

SEQ ID NO: 125 - protein, 103 aa, Arabidopsis thaliana
  misc_feature (1)..(103): 12676237_protein_ID_12676238
EGTTSINPPQAVAAGNVAGNMAGAHGMGSRSMPRPMVAHNMQRMQQSQGMMAYNFPAQAGLNPSVPLQQQRGMAQPHQQQQLRRKDPGMGMSGYAPPNKSRRL

SEQ ID NO: 126 - DNA, 2943 bp, Arabidopsis thaliana
  misc_feature (1)..(2943): 12721583_construct_ID_YP0071
atggcgatgagacttttgaagactcatcttctgtttctgcatctgtatctatttttctcaccatgtttcgcttacactgacatggaagttcttctcaatctcaaatcctccatgattggtcctaaaggacacggtctccacgactggattcactcatcttctccggatgctcactgttctttctccggcgtctcatgtgacgacgatgctcgtgttatctctctcaacgtctccttcactcctttgtttggtacaatctcaccagagattgggatgttgactcatttggtgaatctaactttagctgccaacaacttcaccggtgaattaccattggagatgaagagtctaacttctctcaaggttttgaatatctccaacaatggtaaccttactggaacattccctggagagattttaaaagctatggttgatcttgaagttcttgacacttataacaacaatttcaacggtaagttaccaccggagatgtcagagcttaagaagcttaaatacctctctttcggtggaaatttcttcagcggagagattccagagagttatggagatattcaaagcttagagtatcttggtctcaacggagctggactctccggtaaatctccggcgtttctttcccgcctcaagaacttaagagaaatgtatattggctactacaacagctacaccggtggtgttccaccggagttcggtggtttaacaaagcttgagatcctcgacatggcgagctgtacactcaccggagagattccgacgagtttaagtaacctgaaacatctacatactctgtttcttcacatcaacaacttaaccggtcatataccaccggagctttccggtttagtcagcttgaaatctctcgatttatcaatcaatcagttaaccggagaaatccctcaaagcttcatcaatctcggaaacattactctaatcaatctcttcagaaacaatctctacggacaaataccagaggccatcggagaattaccaaaactcgaagtcttcgaagtatgggagaacaatttcacgttacaattaccggcgaatcttggccggaacgggaatctaataaagcttgatgtctctgataatcatctcaccggacttatccccaaggacttatgcagaggtgagaaattagagatgttaattctctctaacaacttcttctttggtccaattccagaagagcttggtaaatgcaaatccttaaccaaaatcagaatcgttaagaatcttctcaacggcactgttccggcggggcttttcaatctaccgttagttacgattatcgaactcactgataatttcttctccggtgaacttccggtaacgatgtccggcgatgttctcgatcagatttacctctctaacaactggttttccggcgagattccacctgcgattggtaatttccccaatctacagactctattcttagatcggaaccgatttcgcggcaacattccgagagaaatcttcgaattgaagcatttatcgaggatcaacacaagtgcgaacaacatcaccggcggtattccagattcaatctctcgctgctcaactttaatctccgtcgatctcagccgtaaccgaatcaacggagaaatccctaaagggatcaacaacgtgaaaaacttaggaactctaaatatctccggtaatcaattaaccggttcaatccctaccggaatcggaaacatgacgagtttaacaactctcgatctctctttcaacgatctctccggtagagtaccactcggtggtcaattcttggtgttcaacgaaacttccttcgccggaaacacttacctctgtctccctcaccgtgtctcttgtccaacacggccaggacaaacctccgatcacaatcacacggcgttgttctcaccgtcaaggatcgtaatcacggttatcgcagcgatcaccggtttgatcctaatcagtgtagcgattcgtcagatgaataagaagaagaaccagaaatctctcgcctggaaactaaccgccttccagaaactagatttcaaatctgaagacgttctcgagtgtcttaaagaagagaacataatcggtaaaggcggagctggaattgtctaccgtggatcaatgccaaacaacgtagacgtcgcgattaaacgactcgttggccgtgggaccgggaggagcgatcatggattcacggcggagattcaaactttggggagaatccgccaccgtcacatagtgagacttcttggttacgtagcgaacaaggatacgaatctccttctttatgagtacatgcctaatggaagccttggagagcttttgcatggatctaaaggtggtcatcttcaatgggagacgagacatagagtagccgtggaagctgcaaagggcttgtgttatcttcaccatgattgttcaccattgatcttgcatagagatgttaagtccaataacattcttttggactctgattttgaagcccatgttgctgattttgggcttgctaagttcttagttgatggtgctgcttctgagtgtatgtcttcaattgctggctcttatggatacatcgccccagagtatgcatataccttgaaagtggacgagaagagtgatgtgtatagtttcggagtggttttgttggagttaatagctgggaagaaacctgttggtgaatttggagaaggagtggatatagttaggtgggtgaggaacacggaagaggagataactcagccatcggatgctgctattgttgttgcgattgttgacccgaggttgactggttacccgttgacaagtgtgattcatgtgttcaagatcgcaatgatgtgtgtggaggaagaagccgcggcaaggcctacgatgagggaagttgtgcacatgctcactaaccctcctaaatccgtggcgaacttgatcgcgttctga

SEQ ID NO: 127 - protein, 980 aa, Arabidopsis thaliana
  misc_feature (1)..(980): 12721583_protein_ID_12721584
MAMRLLKTHLLFLHLYLFFSPCFAYTDMEVLLNLKSSMIGPKGHGLHDWIHSSSPDAHCSFSGVSCDDDARVISLNVSFTPLFGTISPEIGMLTHLVNLTLAANNFTGELPLEMKSLTSLKVLNISNNGNLTGTFPGEILKAMVDLEVLDTYNNNFNGKLPPEMSELKKLKYLSFGGNFFSGEIPESYGDIQSLEYLGLNGAGLSGKSPAFLSRLKNLREMYIGYYNSYTGGVPPEFGGLTKLEILDMASCTLTGEIPTSLSNLKHLHTLFLHINNLTGHIPPELSGLVSLKSLDLSINQLTGEIPQSFINLGNITLINLFRNNLYGQIPEAIGELPKLEVFEVWENNFTLQLPANLGRNGNLIKLDVSDNHLTGLIPKDLCRGEKLEMLILSNNFFFGPIPEELGKCKSLTKIRIVKNLLNGTVPAGLFNLPLVTIIELTDNFFSGELPVTMSGDVLDQIYLSNNWFSGEIPPAIGNFPNLQTLFLDRNRFRGNIPREIFELKHLSRINTSANNITGGIPDSISRCSTLISVDLSRNRINGEIPKGINNVKNLGTLNISGNQLTGSIPTGIGNMTSLTTLDLSFNDLSGRVPLGGQFLVFNETSFAGNTYLCLPHRVSCPTRPGQTSDHNHTALFSPSRIVITVIAAITGLILISVAIRQMNKKKNQKSLAWKLTAFQKLDFKSEDVLECLKEENIIGKGGAGIVYRGSMPNNVDVAIKRLVGRGTGRSDHGFTAEIQTLGRIRHRHIVRLLGYVANKDTNLLLYEYMPNGSLGELLHGSKGGHLQWETRHRVAVEAAKGLCYLHHDCSPLILHRDVKSNNILLDSDFEAHVADFGLAKFLVDGAASECMSSIAGSYGYIAPEYAYTLKVDEKSDVYSFGVVLLELIAGKKPVGEFGEGVDIVRWVRNTEEEITQPSDAAIVVAIVDPRLTGYPLTSVIHVFKIAMMCVEEEAAARPTMREVVHMLTNPPKSVANLIAF

SEQ ID NO: 128 - DNA, 2454 bp, Arabidopsis thaliana
  misc_feature (1)..(2454): 13593439_construct_ID_YP0122
aagccacacaatctcttttcttctctctctctctgttatatctcttctgtttaattcttttattcttcttcgtctatcttctcctataatctcttctctctccctcttcacctaaagaataagaagaaaaataattcacatctttatgcaaactactttcttgtagggttttaggagctatctctattgtcttggttctgatacaaagttttgtaattttcatggtatgagaagatttgcctttctattttgtttattggttctttttaactttttcttggagatgggttcttgtagatcttaatgaaacttctgtttttgtcccaaaaagagttttcttttttcttctcttctttttgggttttcaattcttgagagacatggcaagagatcagttctatggtcacaataaccatcatcatcaagagcaacaacatcaaatgattaatcagatccaagggtttgatgagacaaaccaaaacccaaccgatcatcatcattacaatcatcagatctttggctcaaactccaacatgggtatgatgatagacttctctaagcaacaacagattaggatgacaagtggttcggatcatcatcatcatcatcatcagacaagtggtggtactgatcagaatcagcttctggaagattcttcatctgccatgagactatgcaatgttaataatgatttcccaagtgaagtaaatgatgagagaccaccacaaagaccaagccaaggtctttccctttctctctcctcttcaaatcctacaagcatcagtctccaatctttcgaactcagaccccaacaacaacaacaacaagggtattccggtaataaatcaacacaacatcagaatctccaacacacgcagatgatgatgatgatgatgaatagtcaccaccaaaacaacaacaataacaatcatcagcatcataatcatcatcagtttcagattgggagttccaagtatttgagtccagctcaagagctactgagtgagttttgcagtcttggagtaaaggaaagcgatgaagaagtgatgatgatgaagcataagaagaagcaaaagggtaaacaacaagaagagtgggacacaagtcaccacagcaacaatgatcaacatgaccaatctgcgactacttcttcaaagaaacatgttccaccacttcactctcttgagttcatggaacttcagaaaagaaaagccaagttgctctccatgctcgaagagcttaaaagaagatatggacattaccgagagcaaatgagagttgcggcggcagcctttgaagcggcggttggactaggaggggcagagatatacactgcgttagcgtcaagggcaatgtcaagacactttcggtgtttaaaagacggacttgtgggacagattcaagcaacaagtcaagctttgggagagagagaagaggataatcgtgcggtttctattgcagcacgtggagaaactccacggttgagattgctcgatcaagctttgcggcaacagaaatcgtatcgccaaatgactcttgttgacgctcatccttggcgtccacaacgcggcttgcctgaacgcgcagtcacaacgttgagagcttggctctttgaacactttcttcacccatatccgagcgatgttgataagcatatattggcccgacaaactggtttatcaagaagtcaggtatcaaattggtttattaatgcaagagttaggctatggaaaccaatgattgaagaaatgtactgtgaagaaacaagaagtgaacaaatggagattacaaacccgatgatgatcgatactaaaccggacccggaccagttgatccgtgtcgaaccggaatctttatcctcaatagtgacaaaccctacatccaaatccggtcacaactcaacccatggaacgatgtcgttagggtcaacgtttgacttttccttgtacggtaaccaagctgtgacatacgctggtgaaggagggccacgtggtgacgtttccttgacgcttgggttacaacgtaacgatggtaacggtggtgtgagtttagcgttgtctccagtgacggctcaaggtggccaacttttctacggtagagaccacattgaagaaggaccggttcaatattcagcgtcgatgttagatgatgatcaagttcagaatttgccttataggaatttgatgggagctcaattacttcatgatattgtttgagattaaaagattaggaccaaagttatcgatacatattttccaaaaccgattcggttatgtaacggtttagttagataaaaaccaaattagatatttatatataccgttgtctgattggattggaggattggtggacaaggagatattattaatgtatgagttagttggttcgtcaatatcacttgtaggatattttcattttgttttttaaaatatattattgagaggtttttttctc

SEQ ID NO: 129 - protein, 611 aa, Arabidopsis thaliana
  misc_feature (1)..(611): 13593439_protein_ID_13593440
MARDQFYGHNNHHHQEQQHQMINQIQGFDETNQNPTDHHHYNHQIFGSNSNMGMMIDFSKQQQIRMTSGSDHHHHHHQTSGGTDQNQLLEDSSSAMRLCNVNNDFPSEVNDERPPQRPSQGLSLSLSSSNPTSISLQSFELRPQQQQQQGYSGNKSTQHQNLQHTQMMMMMMNSHHQNNNNNNHQHHNHHQFQIGSSKYLSPAQELLSEFCSLGVKESDEEVMMMKHKKKQKGKQQEEWDTSHHSNNDQHDQSATTSSKKHVPPLHSLEFMELQKRKAKLLSMLEELKRRYGHYREQMRVAAAAFEAAVGLGGAEIYTALASRAMSRHFRCLKDGLVGQIQATSQALGEREEDNRAVSIAARGETPRLRLLDQALRQQKSYRQMTLVDAHPWRPQRGLPERAVTTLRAWLFEHFLHPYPSDVDKHILARQTGLSRSQVSNWFINARVRLWKPMIEEMYCEETRSEQMEITNPMMIDTKPDPDQLIRVEPESLSSIVTNPTSKSGHNSTHGTMSLGSTFDFSLYGNQAVTYAGEGGPRGDVSLTLGLQRNDGNGGVSLALSPVTAQGGQLFYGRDHIEEGPVQYSASMLDDDQVQNLPYRNLMGAQLLHDIV

SEQ ID NO: 130 - DNA, 962 bp, Arabidopsis thaliana
  misc_feature (1)..(962): 13612380_construct_ID_YP0015
aaaaaagttcagatatttgataaatcaatcaacaaaacaaaaaaaactctatagttagtttctctgaaaatgtacggacagtgcaatatagaatccgactacgctttgttggagtcgataacacgtcacttgctaggaggaggaggagagaacgagctgcgactcaatgagtcaacaccgagttcgtgtttcacagagagttggggaggtttgccattgaaagagaatgattcagaggacatgttggtgtacggactcctcaaagatgccttccattttgacacgtcatcatcggacttgagctgtctttttgattttccggcggttaaagtcgagccaactgagaactttacggcgatggaggagaaaccaaagaaagcgataccggttacggagacggcagtgaaggcgaagcattacagaggagtgaggcagagaccgtgggggaaattcgcggcggagatacgtgatccggcgaagaatggagctagggtttggttagggacgtttgagacggcggaagatgcggctttagcttacgatatagctgcttttaggatgcgtggttcccgcgctttattgaattttccgttgagggttaattccggtgaacctgacccggttcggatcacgtctaagagatcttcttcgtcgtcgtcgtcgtcgtcctcttctacgtcgtcgtctgaaaacgggaagttgaaacgaaggagaaaagcagagaatctgacgtcggaggtggtgcaggtgaagtgtgaggttggtgatgagacacgtgttgatgagttattggtttcataagtttgatcttgtgtgttttgtagttgaatagttttgctataaatgttgaggcaccaagtaaaagtgttcccgtgatgtaaattagttactaaacagagccatatatcttcaatccataaacaaaatagacactttaataaagccgtgagtgttaatttttc

SEQ ID NO: 131 - protein, 243 aa, Arabidopsis thaliana
  misc_feature (1)..(243): 13612380_protein_ID_13612381
MYGQCNIESDYALLESITRHLLGGGGENELRLNESTPSSCFTESWGGLPLKENDSEDMLVYGLLKDAFHFDTSSSDLSCLFDFPAVKVEPTENFTAMEEKPKKAIPVTETAVKAKHYRGVRQRPWGKFAAEIRDPAKNGARVWLGTFETAEDAALAYDIAAFRMRGSRALLNFPLRVNSGEPDPVRITSKRSSSSSSSSSSSTSSSENGKLKRRRKAENLTSEVVQVKCEVGDETRVDELLVS

SEQ ID NO: 132 - DNA, 19 bp, Artificial Sequence
  oligo(dT)18 primer
ttttttttttttttttttv

SEQ ID NO: 133 - DNA, 19 bp, Artificial Sequence
  oligo dTV primer
ttttttttttttttttttn

(19) United States
(12) Patent Application Publication
(10) Pub. No.: US 2006/0015970 A1
(43) Pub. Date: Jan. 19, 2006

(54) Nucleotide sequences and polypeptides encoded thereby useful for modifying plant characteristics

(75) Inventors: Roger Pennell, Malibu, CA (US); Jack Okamuro, Oak Park, CA (US); Richard Schneeberger, Van Nuys, CA (US); Yiwen Fang, Los Angeles, CA (US); Shing Kwok, Woodland Hills, CA (US); Diane Jofuku, Arlington, VA (US); Edward A. Kiegle, Chester, VT (US); Jonathan Donson, Oak Park, CA (US); Nestor Apuya, Culver City, CA (US)

Correspondence Address: BIRCH STEWART KOLASCH & BIRCH, PO BOX 747, FALLS CHURCH, VA, 22040-0747, US

(73) Assignee: Cers, Inc., Thousand Oaks, CA (US)

(21) Appl. No.: 11/010,239

(22) Filed: Dec. 9, 2004

Related U.S. Application Data
(60) Provisional application No. 60/529,352, filed on Dec. 12, 2003.

(51) Int. Cl. A01H 1/00; C12N 15/82; C07H 21/04; C07K 14/415; C12N 5/04

(52) U.S. Cl. 800/288; 435/419; 435/468; 530/370; 536/23.6

(57) ABSTRACT

Isolated polynucleotides and polypeptides encoded thereby are described, together with the use of those products for making transgenic plants.

This Nonprovisional application claims priority under 35 U.S.C. § 119(e) on U.S. Provisional Application No(s). 60/529,352 filed on Dec. 12, 2003, the entire contents of which are hereby incorporated by reference.

### FIELD OF THE INVENTION

The present invention relates to isolated polynucleotides, polypeptides encoded thereby, and the use of those products for making transgenic plants.

### BACKGROUND OF THE INVENTION

There are more than 300,000 species of plants. They show a wide diversity of forms, ranging from delicate liverworts, adapted for life in a damp habitat, to cacti, capable of surviving in the desert. The plant kingdom includes herbaceous plants, such as corn, whose life cycle is measured in months, to the giant redwood tree, which can live for thousands of years. This diversity reflects the adaptations of plants to survive in a wide range of habitats. This is seen most clearly in the flowering plants (phylum Angiospermophyta), which are the most numerous, with over 250,000 species. They are also the most widespread, being found from the tropics to the arctic.

The process of plant breeding involving man's intervention in natural breeding and selection is some 20,000 years old. It has produced remarkable advances in adapting existing species to serve new purposes. The world's economics was largely based on the successes of agriculture for most of these 20,000 years.

Plant breeding involves choosing parents, making crosses to allow recombination of gene (alleles) and searching for and selecting improved forms. Success depends on the genes/alleles available, the combinations required and the ability to create and find the correct combinations necessary to give the desired properties to the plant. Molecular genetics technologies are now capable of providing new genes, new alleles and the means of creating and selecting plants with the new, desired characteristics.

Great agronomic value can result from modulating the size of a plant as a whole or of any of its organs. For example, the green revolution came about as a result of creating dwarf wheat plants, which produced a higher seed yield than taller plants because they could withstand higher levels and inputs of fertilizer and water. Modulation of the size and stature of an entire plant or a particular portion of a plant allows productions of plants specifically improved for agriculture, horticulture and other industries. For example, reductions in height of specific ornamentals, crops and tree species can be beneficial, while increasing height of others may be beneficial.

Increasing the length of the floral stems of cut flowers in some species would also be useful, while increasing leaf size in others would be economically attractive. Enhancing the size of specific plant parts, such as seeds and fruit, to enhance yields by specifically stimulating hormone (e.g. Brassinolide) synthesis in these cells is beneficial. Another application is to stimulate early flowering by altering levels of gibberellic acid in specific cells. Changes in organ size and biomass also results in changes in the mass of constituent molecules.

To summarize, molecular genetic technologies provide the ability to modulate and manipulate plant size and stature of the entire plant as well as at the cell, tissue and organ levels. Thus, plant morphology can be altered to maximize the desired plant trait.

### SUMMARY OF THE INVENTION

The present invention, therefore, relates to isolated polynucleotides, polypeptides encoded thereby, and the use of those products for making transgenic plants.

The present invention also relates to processes for increasing the yield in plants, recombinant nucleic acid molecules and polypeptides used for these processes, their uses as well as to plants with an increased yield.

In the field of agriculture and forestry constantly efforts are being made to produce plants with an increased yield, in particular in order to guarantee the supply of the constantly increasing world population with food and to guarantee the supply of reproducible raw materials. Conventionally, it is tried to obtain plants with an increased yield by breeding, which is, however time-consuming and labor-intensive. Furthermore, appropriate breeding programs have to be performed for each relevant plant species.

Progress has partly been made by the genetic manipulation of plants, that is by introducing into and expressing recombinant nucleic acid molecules in plants. Such approaches have the advantage of usually not being limited to one plant species but being transferable to other plant species. In EP-A 0 511 979, e.g., it was described that the expression of a prokaryotic asparagine synthetase in plant cells inter alia leads to an increased biomass production. In WO 96/21737, e.g., the production of plants with an increased yield by the expression of deregulated or unregulated fructose-1,6-bisphosphatase due to the increase of the photosynthesis rate is described. Nevertheless, there still is a need of generally applicable processes for improving the yield in plants interesting for agriculture or forestry. Therefore, the present invention relates to a process for increasing the yield in plants, characterized in that recombinant DNA molecules stably integrated into the genome of plants are expressed.

It was surprisingly found that the expression of the proteins according to the invention specifically leads to an increase in yield.

The term “increase in yield” preferably relates to an increase of the biomass production, in particular when determined as the fresh weight of the plant. Such an increase in yield preferably refers to the so-called “sink” organs of the plant, which are the organs that take up the photoassimilates produced during photosynthesis. Particularly preferred are parts of plants which can be harvested, such as seeds, fruits, storage roots, roots, tubers, flowers, buds, shoots, stems or wood. The increase in yield according to the invention is at least 3% with regard to the biomass in comparison to non-transformed plants of the same genotype when cultivated under the same conditions, preferably at least 10% and particularly preferred at least 20%.

### BRIEF DESCRIPTION OF THE INDIVIDUAL TABLES

Table 1—Polynucleotide and Polypeptide Sequences

Table 1 sets forth the specific polynucleotide and polypeptide sequence of the invention. Each sequence is provided a “cDNA” or “polypeptide” number that directly follows a “>” symbol. A “construct” or “protein/polypeptide” identifier then follows. The description of the sequence directly follows on the next line in Table 1. It will be noted that a polynucleotide sequence is directly followed by the encoded polypeptide sequence.

The “cDNA number” is a number that identifies the sequence used in the experiments. The “construct” text identifies the construct used to produce a specific plant line that allows identification of the expression pattern of the cDNA. This was accomplished by isolating the cDNA's endogenous promoter, operably linking it to Green Flourescent Protein (GFP), transforming plants and microscopically monitoring GFP expression.

Table 2—GFP Expression Reports

Table 2 consists of the GFP Expression Reports and provides details for expression driven by each of the cDNA's endogenous promoter sequence as observed in transgenic plants. The results are presented as summaries of the spatial expression, which provides information as to gross and/or specific expression in various plant organs and tissues. The observed expression pattern is also presented, which gives details of expression during different generations or different developmental stages within a generation. Additional information is provided regarding the associated gene, the GenBank reference, the source organism of the promoter, and the vector and marker genes used for the construct. The following symbols are used consistently throughout the Table:
T1: First generation transformant
T2: Second generation transformant
T3: Third generation transformant
(L): low expression level
(M): medium expression level
(H): high expression level

Each report in Table 2 identifies a construct and the promoter's endogenous cDNA, the sequence of which is described in Table 1.

Table 3—Microarray Expression

Table 3 presents the results of microarray experiments that track expression of the cDNAs under specific conditions and under the control of their respective endogenous promoters. The column headed “cDNA_ID” provides the identifier number for the cDNA tracked in the experiment. Using Table 2, these numbers can be used to correlate the differential expression pattern observed and produced by the cDNA of the invention driven by its endogenous promoter and with the cDNA of the invention's endogenous promoter driving green fluorescent protein (GFP) expression.

The column headed “EXPT_REP_ID” provides an identifier number for the particular experiment conducted. The column “SHORT_NAME” gives a brief description of the experimental conditions or the developmental stage used. The values in the column headed “Differential” indicate whether expression of the cDNA was increased (+) or decreased (−) compared to the control.

Table 4—Associated Utility

Table 4 links the “short name” from Table 4 with the title of a utility section set forth in the Specification.

### DETAILED DESCRIPTION OF THE INVENTION

1. Definitions

The following terms are utilized throughout this application:

Allelic variant: An “allelic variant” is an alternative form of the same SDF, which resides at the same chromosomal locus in the organism. Allelic variations can occur in any portion of the gene sequence, including regulatory regions. Allelic variants can arise by normal genetic variation in a population. Allelic variants can also be produced by genetic engineering methods. An allelic variant can be one that is found in a naturally occurring plant, including a cultivar or ecotype. An allelic variant may or may not give rise to a phenotypic change, and may or may not be expressed. An allele can result in a detectable change in the phenotype of the trait represented by the locus. A phenotypically silent allele can give rise to a product.

Chimeric: The term “chimeric” is used to describe genes, as defined supra, or contructs wherein at least two of the elements of the gene or construct, such as the promoter and the coding sequence and/or other regulatory sequences and/or filler sequences and/or complements thereof, are heterologous to each other.

Constitutive Promoter: Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

Coordinately Expressed: The term “coordinately expressed,” as used in the current invention, refers to genes that are expressed at the same or a similar time and/or stage and/or under the same or similar environmental conditions.

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s).

Endogenous: The term “endogenous,” within the context of the current invention refers to any polynucleotide, polypeptide or protein sequence which is a natural part of a cell or organisms regenerated from said cell. In the context of this application, the phrase “endogenous promoter” refers to the promoter that is naturally operably linked to a particular cDNA, while “endogenous coding region” or “endogenous cDNA” refers to the coding region that is naturally operably linked to a specific promoter.

Exogenous: “Exogenous,” as referred to within, is any polynucleotide, polypeptide or protein sequence, whether chimeric or not, that is initially or subsequently introduced into the genome of an individual host cell or the organism regenerated from said host cell by any means other than by a sexual cross. Examples of means by which this can be accomplished are described below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al., Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

Gene: The term “gene,” as used in the context of the current invention, encompasses all regulatory and coding sequence contiguously associated with a single hereditary unit with a genetic function. Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes comprised of “exons” (coding sequences), which may be interrupted by “introns” (non-coding sequences), encode proteins. A gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, artificial chromosome, plasmid, vector, etc., or as a separate isolated entity.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence originates from, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Homologous gene: In the current invention, “homologous gene” refers to a gene that shares sequence similarity with the gene of interest. This similarity may be in only a fragment of the sequence and often represents a functional domain such as, examples including without limitation a DNA binding domain, a domain with tyrosine kinase activity, or the like. The functional activities of homologous genes are not necessarily the same.

Inducible Promoter: An “inducible promoter” in the context of the current invention refers to a promoter which is regulated under certain conditions, such as light, chemical concentration, protein concentration, conditions in an organism, cell, or organelle, etc. A typical example of an inducible promoter, which can be utilized with the polynucleotides of the present invention, is PARSK1, the promoter from the Arabidopsis gene encoding a serine-threonine kinase enzyme, and which promoter is induced by dehydration, abscissic acid and sodium chloride (Wang and Goodman, Plant J. 8:37 (1995)). Examples of environmental conditions that may affect transcription by inducible promoters include anaerobic conditions, elevated temperature, or the presence of light.

Modulate Transcription Level: As used herein, the phrase “modulate transcription” describes the biological activity of a promoter sequence or promoter control element. Such modulation includes, without limitation, includes up- and down-regulation of initiation of transcription, rate of transcription, and/or transcription levels.

Mutant: In the current invention, “mutant” refers to a heritable change in nucleotide sequence at a specific location. Mutant genes of the current invention may or may not have an associated identifiable phenotype.

Operable Linkage: An “operable linkage” is a linkage in which a promoter sequence or promoter control element is connected to a polynucleotide sequence (or sequences) in such a way as to place transcription of the polynucleotide sequence under the influence or control of the promoter or promoter control element. Two DNA sequences (such as a polynucleotide to be transcribed and a promoter sequence linked to the 5′ end of the polynucleotide to be transcribed) are said to be operably linked if induction of promoter function results in the transcription of mRNA encoding the polynucleotide and if the nature of the linkage between the two DNA sequences does not (1) result in the introduction of a frame-shift mutation, (2) interfere with the ability of the promoter sequence to direct the expression of the protein, antisense RNA or ribozyme, or (3) interfere with the ability of the DNA template to be transcribed. Thus, a promoter sequence would be operably linked to a polynucleotide sequence if the promoter was capable of effecting transcription of that polynucleotide sequence.

Orthologous: In the current invention “orthologous gene” refers to a second gene that encodes a gene product that performs a similar function as the product of a first gene. The orthologous gene may also have a degree of sequence similarity to the first gene. The orthologous gene may encode a polypeptide that exhibits a degree of sequence similarity to a polypeptide corresponding to a first gene. The sequence similarity can be found within a functional domain or along the entire length of the coding sequence of the genes and/or their corresponding polypeptides.

“Orthologous” is also a term used herein to describe a relationship between two or more polynucleotides or proteins. Two polynucleotides or proteins are “orthologous” to one another if they serve a similar function in different organisms. In general, orthologous polynucleotides or proteins will have similar catalytic functions (when they encode enzymes) or will serve similar structural functions (when they encode proteins or RNA that form part of the ultrastructure of a cell).

Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

Plant Promoter: A “plant promoter” is a promoter capable of initiating transcription in plant cells and can drive or facilitate transcription of a fragment of the SDF of the instant invention or a coding sequence of the SDF of the instant invention. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1) promoter known to those of skill.

Plant Tissue: The term “plant tissue” includes differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

Preferential Transcription: “Preferential transcription” is defined as transcription that occurs in a particular pattern of cell types or developmental times or in response to specific stimuli or combination thereof. Non-limitive examples of preferential transcription include: high transcript levels of a desired sequence in root tissues; detectable transcript levels of a desired sequence in certain cell types during embryogenesis; and low transcript levels of a desired sequence under drought conditions. Such preferential transcription can be determined by measuring initiation, rate, and/or levels of transcription.

Promoter: The term “promoter,” as used herein, refers to a region of sequence determinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “CCAAT box” element (typically a sequence CCAAT) and/or a GGGCG sequence, usually located between 40 and 200 nucleotides, preferably 60 to 120 nucleotides, upstream from the start site of transcription.

Public sequence: The term “public sequence,” as used in the context of the instant application, refers to any sequence that has been deposited in a publicly accessible database prior to the filing date of the present application. This term encompasses both amino acid and nucleotide sequences. Such sequences are publicly accessible, for example, on the BLAST databases on the NCBI FTP web site (accessible at ncbi.nlm.nih.gov/ftp). The database at the NCBI FTP site utilizes “gi” numbers assigned by NCBI as a unique identifier for each sequence in the databases, thereby providing a non-redundant database for sequence from various databases, including GenBank, EMBL, DBBJ, (DNA Database of Japan) and PDB (Brookhaven Protein Data Bank).

Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.

Signal Peptide: A “signal peptide” as used in the current invention is an amino acid sequence that targets the protein for secretion, for transport to an intracellular compartment or organelle or for incorporation into a membrane. Signal peptides are indicated in the tables and a more detailed description located below.

Specific Promoter: In the context of the current invention, “specific promoters” refers to a subset of inducible promoters that have a high preference for being induced in a specific tissue or cell and/or at a specific time during development of an organism. By “high preference” is meant at least 3-fold, preferably 5-fold, more preferably at least 10-fold still more preferably at least 20-fold, 50-fold or 100-fold increase in transcription in the desired tissue over the transcription in any other tissue. Typical examples of temporal and/or tissue specific promoters of plant origin that can be used with the polynucleotides of the present invention, are: PTA29, a promoter which is capable of driving gene transcription specifically in tapetum and only during anther development (Koltonow et al., Plant Cell 2:1201 (1990); RCc2 and RCc3, promoters that direct root-specific gene transcription in rice (Xu et al., Plant Mol. Biol. 27:237 (1995); TobRB27, a root-specific promoter from tobacco (Yamamoto et al., Plant Cell 3:371 (1991)). Examples of tissue-specific promoters under developmental control include promoters that initiate transcription only in certain tissues or organs, such as root, ovule, fruit, seeds, or flowers. Other suitable promoters include those from genes encoding storage proteins or the lipid body membrane protein, oleosin. A few root-specific promoters are noted above.

Stringency: “Stringency” as used herein is a function of probe length, probe composition (G+C content), and salt concentration, organic solvent concentration, and temperature of hybridization or wash conditions. Stringency is typically compared by the parameter Tₘ, which is the temperature at which 50% of the complementary molecules in the hybridization are hybridized, in terms of a temperature differential from Tₘ. High stringency conditions are those providing a condition of Tₘ—5° C. to Tₘ—10° C. Medium or moderate stringency conditions are those providing Tₘ—20° C. to Tₘ—29° C. Low stringency conditions are those providing a condition of Tₘ—40° C. to Tₘ—48° C. The relationship of hybridization conditions to Tₘ (in ° C.) is expressed in the mathematical equation
Tₘ=81.5−16.6(log₁₀[Na⁺])+0.41(% G+C)−(600/N) (1)
where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).
Tₘ=81.5+16.6 log{[Na⁺]/(1+0.7[Na⁺])}+0.41(% G+C)−500/L0.63(% formamide) (2)
where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene or DNA sequence can be considered substantially free of other plant genes or DNA sequences.

Suppressor: See “Enhancer/Suppressor”

TATA to start: “TATA to start” shall mean the distance, in number of nucleotides, between the primary TATA motif and the start of transcription.

Transgenic plant: A “transgenic plant” is a plant having one or more plant cells that contain at least one exogenous polynucleotide introduced by recombinant nucleic acid methods.

Translational start site: In the context of the current invention, a “translational start site” is usually an ATG in the cDNA transcript, more usually the first ATG. A single cDNA, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can intiate at one or more sites within the gene, and a single gene may have multiple transcriptional start sites, some of which may be specific for transcription in a particular cell-type or tissue.

Untranslated region (UTR): A “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).

Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

2. Important Characteristics of the Polynuceotides of the Invention

The genes and polynucleotides of the present invention are of interest because when they are misexpressed (i.e. when expressed at a non-natural location or in an increased amount) they produce plants with modified characteristics as discussed below as evidenced by the results of differential expression experiments. These traits can be used to exploit or maximize plant products. For example, an increase in plant height is beneficial in species grown or harvested for their main stem or trunk, such as ornamental cut flowers, fiber crops (e.g. flax, kenaf, hesperaloe, hemp) and wood producing trees. Increase in inflorescence thickness is also desirable for some ornamentals, while increases in the number and size of leaves can lead to increased production/harvest from leaf crops such as lettuce, spinach, cabbage and tobacco.

3. The Genes of the Invention

The sequences of the invention were isolated from Arabidopsis thaliana.

4. Use of the Genes to Make Transgenic Plants

To use the sequences of the present invention or a combination of them or parts and/or mutants and/or fusions and/or variants of them, recombinant DNA constructs are prepared which comprise the polynucleotide sequences of the invention inserted into a vector, and which are suitable for transformation of plant cells. The construct can be made using standard recombinant DNA techniques (Sambrook et al. 1989) and can be introduced to the species of interest by Agrobacterium-mediated transformation or by other means of transformation as referenced below.

The vector backbone can be any of those typical in the art such as plasmids, viruses, artificial chromosomes, BACs, YACs and PACs and vectors of the sort described by
(a) BAC: Shizuya et al., Proc. Natl. Acad. Sci. USA 89: 8794-8797 (1992); Hamilton et al., Proc. Natl. Acad. Sci. USA 93: 9975-9979 (1996);
(b) YAC: Burke et al., Science 236:806-812 (1987);
(c) PAC: Stemberg N. et al., Proc Natl Acad Sci USA. January; 87(1):103-7 (1990);
(d) Bacteria-Yeast Shuttle Vectors: Bradshaw et al., Nucl Acids Res 23: 4850-4856 (1995);
(e) Lambda Phage Vectors: Replacement Vector, e.g., Frischauf et al., J. Mol. Biol. 170: 827-842 (1983); or Insertion vector, e.g., Huynh et al., In: Glover NM (ed) DNA Cloning: A practical Approach, Vol. 1 Oxford: IRL Press (1985); T-DNA gene fusion vectors Walden et al., Mol Cell Biol 1: 175-194 (1990); and
(g) Plasmid vectors: Sambrook et al., infra.

Typically, the construct will comprise a vector containing a sequence of the present invention with any desired transcriptional and/or translational regulatory sequences, such as promoters, UTRs, and 3′ end termination sequences. Vectors can also include origins of replication, scaffold attachment regions (SARs), markers, homologous sequences, introns, etc. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin.

A plant promoter fragment may be used that directs transcription of the gene in all tissues of a regenerated plant and may be a constitutive promoter, such as 355. Alternatively, the plant promoter may direct transcription of a sequence of the invention in a specific tissue (tissue-specific promoters) or may be otherwise under more precise environmental control (inducible promoters).

If proper polypeptide production is desired, a polyadenylation region at the 3′-end of the coding region is typically included. The polyadenylation region can be derived from the natural gene, from a variety of other plant genes, or from T-DNA.

Knock-In Constructs

Ectopic expression of the sequences of the invention can also be accomplished using a “knock-in” approach. Here, the first component, an “activator line,” is created by generating a transgenic plant comprising a transcriptional activator operatively linked to a promoter. The second component comprises the desired cDNA sequence operatively linked to the target binding sequence/region of the transcriptional activator. The second component can be transformed into the “activator line” or be used to transform a host plant to produce a “target” line that can be crossed with the “activator line” by ordinary breeding methods. In either case, the result is the same. That is, the promoter drives production of the transcriptional activator protein that then binds to the target binding region to facilitate expression of the desired cDNA.

Any promoter that functions in plants can be used in the first component, such as the 35S Cauliflower Mosaic Virus promoter or a tissue or organ specific promoter. Suitable transcriptional activator polypeptides include, but are not limited to, those encoding HAP1 and GAL4. The binding sequence recognized and targeted by the selected transcriptional activator protein is used in the second component.

Transformation

Techniques for transforming a wide variety of higher plant species are well known and described in the technical and scientific literature. See, e.g. Weising et al., Ann. Rev. Genet. 22:421 (1988); and Christou, Euphytica, v. 85, n.1-3:13-27, (1995).

Processes for the transformation of monocotyledonous and dicotyledonous plants are known to the person skilled in the art. For the introduction of DNA into a plant host cell a variety of techniques is available. These techniques comprise the transformation of plant cells with T-DNA using Agrobacterium tumefaciens or Agrobacterium rhizogenes as transformation means, the fusion of protoplasts, the injection, the electroporation of DNA, the introduction of DNA by means of the biolistic method as well as further possibilities.

For the injection and electroporation of DNA in plant cells the plasmids do not have to fulfill specific requirements. Simple plasmids such as pUC derivatives can be used.

The use of agrobacteria for the transformation of plant cells has extensively been examined and sufficiently disclosed in the specification of EP-A 120 516, in Hoekema (In: The Binary Plant Vector System Offsetdrulkkerij Kanters B. V., Alblasserdam (1985), Chapter V), Fraley et al. (Crit. Rev. Plant. Sci. 4, 1-46) and An et al. (EMBO J. 4 (1985), 277-287).

For the transfer of the DNA to the plant cell plant explants can be co-cultivated with Agrobacterium tumefaciens or Agrobacterium rhizogenes. From the infected plant material (for example leaf explants, segments of stems, roots but also protoplasts or suspension cultivated plant cells) whole plants can be regenerated in a suitable medium which may contain antibiotics or biozides for the selection of transformed cells. The plants obtained that way can then be examined for the presence of the introduced DNA. Other possibilities for the introduction of foreign DNA using the biolistic method or by protoplast transformation are known (cf., e.g., Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

The transformation of dicotyledonous plants via Ti-plasmid-vector systems with the help of Agrobacterium tumefaciens is well-established. Recent studies have indicated that also monocotyledonous plants can be transformed by means of vectors based on Agrobacterium (Chan et al., Plant Mol. Biol. 22 (1993), 491-506; Hiei et al., Plant J. 6 (1994), 271-282; Deng et al., Science in China 33 (1990), 28-34; Wilmink et al., Plant Cell Reports 11 (1992), 76-80; May et al., Bio/Technology 13 (1995), 486-492; Conner and Domisse; Int. J. Plant Sci. 153 (1992), 550-555; Ritchie et al., Transgenic Res. 2 (1993), 252-265).

Alternative systems for the transformation of monocotyledonous plants are the transformation by means of the biolistic method (Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631), the protoplast transformation, the electroporation of partially permeabilized cells, as well as the introduction of DNA by means of glass fibers.

In particular the transformation of maize is described in the literature several times (cf., e.g., WO95/06128, EP 0 513 849; EP 0 465 875; Fromm et al., Biotechnology 8 (1990), 833-844; Gordon-Kamm et al., Plant Cell 2 (1990), 603-618; Koziel et al., Biotechnology 11 (1993), 194-200). In EP 292 435 and in Shillito et al. (Bio/Technology 7 (1989), 581) a process is described with the help of which and starting from a mucus-free, soft (friable) maize callus fertile plants can be obtained. Prioli and Söndahl (Bio/Technology 7 (1989), 589) describe the regenerating and obtaining of fertile plants from maize protoplasts of the Cateto maize inbred line Cat 100-1.

The successful transformation of other cereal species has also been described, for example for barley (Wan and Lemaux, see above; Ritala et al., see above) and for wheat (Nehra et al., Plant J. 5 (1994), 285-297).

Once the introduced DNA has been integrated into the genome of the plant cell, it usually is stable there and is also contained in the progenies of the originally transformed cell. It usually contains a selection marker which makes the transformed plant cells resistant to a biozide or an antibiotic such as kanamycin, G 418, bleomycin, hygromycin or phosphinotricin and others. Therefore, the individually chosen marker should allow the selection of transformed cells from cells lacking the introduced DNA.

The transformed cells grow within the plant in the usual way (see also McCormick et al., Plant Cell Reports 5 (1986), 81-84). The resulting plants can be cultured normally. Seeds can be obtained from the plants.

Two or more generations should be cultivated to make sure that the phenotypic feature is maintained stably and is transmitted. Seeds should be harvested to make sure that the corresponding phenotype or other properties are maintained.

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria (McCormac et al., Mol. Biotechnol. 8:199 (1997); Hamilton, Gene 200:107 (1997)); Salomon et al. EMBO J. 3:141 (1984); Herrera-Estrella et al. EMBO J. 2:987 (1983).

Microinjection techniques are known in the art and well described in the scientific and patent literature. The introduction of DNA constructs using polyethylene glycol precipitation is described in Paszkowski et al. EMBO J. 3:2717 (1984). Electroporation techniques are described in Fromm et al. Proc. Natl. Acad. Sci. USA 82:5824 (1985). Ballistic transformation techniques are described in Klein et al. Nature 327:773 (1987). Agrobacterium tumefaciens-mediated transformation techniques, including disarming and use of binary or co-integrate vectors, are well described in the scientific literature. See, for example Hamilton, C M., Gene 200:107 (1997); Müller et al. Mol. Gen. Genet. 207:171 (1987); Komari et al. Plant J 10:165 (1996); Venkateswarlu et al. Biotechnology 9:1103 (1991) and Gleave, A P., Plant Mol. Biol. 20:1203 (1992); Graves and Goldman, Plant Mol. Biol. 7:34 (1986) and Gould et al., Plant Physiology 95:426 (1991).

Transformed plant cells that have been obtained by any of the above transformation techniques can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture in “Handbook of Plant Cell Culture,” pp. 124-176, MacMillan Publishing Company, New York, 1983; and Binding, Regeneration of Plants, Plant Protoplasts, pp. 21-73, CRC Press, Boca Raton, 1988. Regeneration can also be obtained from plant callus, explants, organs, or parts thereof. Such regeneration techniques are described generally in Klee et al. Ann. Rev. of Plant Phys. 38:467 (1987). Regeneration of monocots (rice) is described by Hosoyama et al. (Biosci. Biotechnol. Biochem. 58:1500 (1994)) and by Ghosh et al. (J. Biotechnol. 32:1 (1994)). The nucleic acids of the invention can be used to confer the trait of increased height, increased primary inflorescence thickness, an increase in the number and size of leaves and a delay in flowering time, without reduction in fertility, on essentially any plant.

The nucleotide sequences according to the invention can generally encode any appropriate proteins from any organism, in particular from plants, fungi, bacteria or animals. The sequences preferably encode proteins from plants or fungi. Preferably, the plants are higher plants, in particular starch or oil storing useful plants, for example potato or cereals such as rice, maize, wheat, barley, rye, triticale, oat, millet, etc., as well as spinach, tobacco, sugar beet, soya, cotton etc.

The process according to the invention can in principle be applied to any plant. Therefore, monocotyledonous as well as dicotyledonous plant species are particularly suitable. The process is preferably used with plants that are interesting for agriculture, horticulture and/or forestry.

Examples thereof are vegetable plants such as, for example, cucumber, melon, pumpkin, eggplant, zucchini, tomato, spinach, cabbage species, peas, beans, etc., as well as fruits such as, for example, pears, apples, etc.

Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and, Zea.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Microarray Analysis

A major way that a cell controls its response to internal or external stimuli is by regulating the rate of transcription of specific genes. For example, the differentiation of cells during organogenensis into forms characteristic of the organ is associated with the selective activation and repression of large numbers of genes. Thus, specific organs, tissues and cells are functionally distinct due to the different populations of mRNAs and protein products they possess. Internal signals program the selective activation and repression programs. For example, internally synthesized hormones produce such signals. The level of hormone can be raised by increasing the level of transcription of genes encoding proteins concerned with hormone synthesis.

To measure how a cell reacts to internal and/or external stimuli, individual mRNA levels can be measured and used as an indicator for the extent of transcription of the gene. Cells can be exposed to a stimulus, and mRNA can be isolated and assayed at different time points after stimulation. The mRNA from the stimulated cells can be compared to control cells that were not stimulated. The mRNA levels that are higher in the stimulated cell versus the control indicate a stimulus-specific response of the cell. The same is true of mRNA levels that are lower in stimulated cells versus the control condition.

Similar studies can be performed with cells taken from an organism with a defined mutation in their genome as compared with cells without the mutation. Altered mRNA levels in the mutated cells indicate how the mutation causes transcriptional changes. These transcriptional changes are associated with the phenotype that the mutated cells exhibit that is different from the phenotype exhibited by the control cells.

Applicants have utilized microarray techniques to measure the levels of mRNAs in cells from plants transformed with the polynucleotides of the invention. In general, transformants with the genes of the invention were grown to an appropriate stage, and tissue samples were prepared for the microarray differential expression analysis.

### EXAMPLE 1

### Microarray Experimental Procedures and Results

Procedures

1. Sample Tissue Preparation

Tissue samples for each of the expression analysis experiments were prepared as follows:

(a) Roots

Seeds of Arabidopsis thaliana (Ws) were sterilized in full strength bleach for less than 5 min., washed more than 3 times in sterile distilled deionized water and plated on MS agar plates. The plates were placed at 4° C. for 3 nights and then placed vertically into a growth chamber having 16 hr light/8 hr dark cycles, 23° C., 70% relative humidity and ˜11,000 LUX. After 2 weeks, the roots were cut from the agar, flash frozen in liquid nitrogen and stored at −80° C.

(b) Rosette Leaves, Stems, and Siliques

Arabidopsis thaliana (Ws) seed was vernalized at 4° C. for 3 days before sowing in Metro-mix soil type 350. Flats were placed in a growth chamber having 16 hr light/8 hr dark, 80% relative humidity, 23° C. and 13,000 LUX for germination and growth. After 3 weeks, rosette leaves, stems, and siliques were harvested, flash frozen in liquid nitrogen and stored at −80° C. until use. After 4 weeks, siliques (<5 mm, 5-10 mm and >10 mm) were harvested, flash frozen in liquid nitrogen and stored at −80° C. until use. 5 week old whole plants (used as controls) were harvested, flash frozen in liquid nitrogen and kept at −80° C. until RNA was isolated.

(c) Germination

Arabidopsis thaliana seeds (ecotype Ws) were sterilized in bleach and rinsed with sterile water. The seeds were placed in 100 mm petri plates containing soaked autoclaved filter paper. Plates were foil-wrapped and left at 4° C. for 3 nights to vernalize. After cold treatment, the foil was removed and plates were placed into a growth chamber having 16 hr light/8 hr dark cycles, 23° C., 70% relative humidity and ˜11,000 lux. Seeds were collected 1 d, 2 d, 3 d and 4 d later, flash frozen in liquid nitrogen and stored at −80° C. until RNA was isolated.

(d) Abscissic Acid (ABA)

Seeds of Arabidopsis thaliana (ecotype Wassilewskija) were sown in trays and left at 4° C. for 4 days to vernalize. They were then transferred to a growth chamber having grown 16 hr light/8 hr dark, 13,000 LUX, 70% humidity, and 20° C. and watered twice a week with 1 L of 1× Hoagland's solution. Approximately 1,000 14 day old plants were spayed with 200-250 mls of 100 μM ABA in a 0.02% solution of the detergent Silwet L-77. Whole seedlings, including roots, were harvested within a 15 to 20 minute time period at 1 hr and 6 hr after treatment, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 100 μM ABA for treatment. Control plants were treated with water. After 6 hr and 24 hr, aerial and root tissues were separated and flash frozen in liquid nitrogen prior to storage at −80° C.

(e) Brassinosteroid Responsive

Two separate experiments were performed, one with epi-brassinolide and one with the brassinosteroid biosynthetic inhibitor brassinazole. In the epi-brassinolide experiments, seeds of wild-type Arabidopsis thaliana (ecotype Wassilewskija) and the brassinosteroid biosynthetic mutant dwf4-1 were sown in trays and left at 4° C. for 4 days to vernalize. They were then transferred to a growth chamber having 16 hr light/8 hr dark, 11,000 LUX, 70% humidity and 22° C. temperature. Four week old plants were spayed with a 1 μM solution of epi-brassinolide and shoot parts (unopened floral primordia and shoot apical meristems) harvested three hours later. Tissue was flash-frozen in liquid nitrogen and stored at −80° C. In the brassinazole experiments, seeds of wild-type Arabidopsis thaliana (ecotype Wassilewskija) were grown as described above. Four week old plants were spayed with a 1 μM solution of brassinazole and shoot parts (unopened floral primordia and shoot apical meristems) harvested three hours later. Tissue was flash-frozen in liquid nitrogen and stored at −80° C.

In addition to the spray experiments, tissue was prepared from two different mutants; (1) a dwf4-1 knock out mutant and (2) a mutant overexpressing the dwf4-1 gene.

Seeds of wild-type Arabidopsis thaliana (ecotype Wassilewskija) and of the dwf4-1 knock out and overexpressor mutants were sown in trays and left at 4° C. for 4 days to vernalize. They were then transferred to a growth chamber having 16 hr light/8 hr dark, 11,000 LUX, 70% humidity and 22° C. temperature. Tissue from shoot parts (unopened floral primordia and shoot apical meristems) was flash-frozen in liquid nitrogen and stored at −80° C.

Another experiment was completed with seeds of Arabidopsis thaliana (ecotype Wassilewskija) were sown in trays and left at 4° C. for 4 days to vernalize. They were then transferred to a growth chamber. Plants were grown under long-day (16 hr light: 8 hr. dark) conditions, 13,000 LUX light intensity, 70% humidity, 20° C. temperature and watered twice a week with 1 L 1× Hoagland's solution (recipe recited in Feldmann et al., (1987) Mol. Gen. Genet. 208: 1-9 and described as complete nutrient solution). Approximately 1,000 14 day old plants were spayed with 200-250 mls of 0.1 μM Epi-Brassinolite in 0.02% solution of the detergent Silwet L-77. At 1 hr. and 6 hrs. after treatment aerial tissues were harvested within a 15 to 20 minute time period and flash-frozen in liquid nitrogen.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 0.1 μM epi-brassinolide for treatment. Control plants were treated with distilled deionized water. After 24 hr, aerial and root tissues were separated and flash frozen in liquid nitrogen prior to storage at −80° C.

(f) Nitrogen: High to Low

Wild type Arabidopsis thaliana seeds (ecotpye Ws) were surface sterilized with 30% Clorox, 0.1% Triton X-100 for 5 minutes. Seeds were then rinsed with 4-5 exchanges of sterile double distilled deionized water. Seeds were vernalized at 4° C. for 2-4 days in darkness. After cold treatment, seeds were plated on modified 1×MS media (without NH₄NO₃ or KNO₃), 0.5% sucrose, 0.5 g/L MES pH5.7, 1% phytagar and supplemented with KNO₃ to a final concentration of 60 mM (high nitrate modified 1×MS media). Plates were then grown for 7 days in a Percival growth chamber at 22° C. with 16 hr. light/8 hr dark.

Germinated seedlings were then transferred to a sterile flask containing 50 mL of high nitrate modified 1×MS liquid media. Seedlings were grown with mild shaking for 3 additional days at 22° C. in 16 hr. light/8 hr dark (in a Percival growth chamber) on the high nitrate modified 1×MS liquid media.

After three days of growth on high nitrate modified 1×MS liquid media, seedlings were transferred either to a new sterile flask containing 50 mL of high nitrate modified 1×MS liquid media or to low nitrate modified 1×MS liquid media (containing 20 □M KNO₃). Seedlings were grown in these media conditions with mild shaking at 22° C. in 16 hr light/8 hr dark for the appropriate time points and whole seedlings harvested for total RNA isolation via the Trizol method (LifeTech.). The time points used for the microarray experiments were 10 min. and 1 hour time points for both the high and low nitrate modified 1×MS media.

Alternatively, seeds that were surface sterilized in 30% bleach containing 0.1% Triton X-100 and further rinsed in sterile water, were planted on MS agar, (0.5% sucrose) plates containing 50 mM KNO₃ (potassium nitrate). The seedlings were grown under constant light (3500 LUX) at 22° C. After 12 days, seedlings were transferred to MS agar plates containing either 1 mM KNO₃ or 50 mM KNO₃. Seedlings transferred to agar plates containing 50 mM KNO₃ were treated as controls in the experiment. Seedlings transferred to plates with 1 mM KNO₃ were rinsed thoroughly with sterile MS solution containing 1 mM KNO₃. There were ten plates per transfer. Root tissue was collected and frozen in 15 mL Falcon tubes at various time points which included 1 hour, 2 hours, 3 hours, 4 hours, 6 hours, 9 hours, 12 hours, 16 hours, and 24 hours.

Maize 35A19 Pioneer hybrid seeds were sown on flats containing sand and grown in a Conviron growth chamber at 25° C., 16 hr light/8 hr dark, ˜13,000 LUX and 80% relative humidity. Plants were watered every three days with double distilled deionized water. Germinated seedlings are allowed to grow for 10 days and were watered with high nitrate modified 1×MS liquid media (see above). On day 11, young corn seedlings were removed from the sand (with their roots intact) and rinsed briefly in high nitrate modified 1×MS liquid media. The equivalent of half a flat of seedlings were then submerged (up to their roots) in a beaker containing either 500 mL of high or low nitrate modified 1×MS liquid media (see above for details).

At appropriate time points, seedlings were removed from their respective liquid media, the roots separated from the shoots and each tissue type flash frozen in liquid nitrogen and stored at −80° C. This was repeated for each time point. Total RNA was isolated using the Trizol method (see above) with root tissues only.

Corn root tissues isolated at the 4 hr and 16 hr time points were used for the microarray experiments. Both the high and low nitrate modified 1×MS media were used.

(g) Nitrogen: Low to High

Arabidopsis thaliana ecotype Ws seeds were sown on flats containing 4 L of a 1:2 mixture of Grace Zonolite vermiculite and soil. Flats were watered with 3 L of water and vernalized at 4° C. for five days. Flats were placed in a Conviron growth chamber having 16 hr light/8 hr dark at 20° C., 80% humidity and 17,450 LUX. Flats were watered with approximately 1.5 L of water every four days. Mature, bolting plants (24 days after germination) were bottom treated with 2 L of either a control (100 mM mannitol pH 5.5) or an experimental (50 mM ammonium nitrate, pH 5.5) solution. Roots, leaves and siliques were harvested separately 30, 120 and 240 minutes after treatment, flash frozen in liquid nitrogen and stored at −80° C.

Hybrid maize seed (Pioneer hybrid 35A19) were aerated overnight in deionized water. Thirty seeds were plated in each flat, which contained 4 liters of Grace zonolite vermiculite. Two liters of water were bottom fed and flats were kept in a Conviron growth chamber with 16 hr light/8 hr dark at 20° C. and 80% humidity. Flats were watered with 1 L of tap water every three days. Five day old seedlings were treated as described above with 2 L of either a control (100 mM mannitol pH 6.5) solution or 1 L of an experimental (50 mM ammonium nitrate, pH 6.8) solution. Fifteen shoots per time point per treatment were harvested 10, 90 and 180 minutes after treatment, flash frozen in liquid nitrogen and stored at −80° C.

Alternatively, seeds of Arabidopsis thaliana (ecotype Wassilewskija) were left at 4° C. for 3 days to vernalize. They were then sown on vermiculite in a growth chamber having 16 hours light/8 hours dark, 12,000-14,000 LUX, 70% humidity, and 20° C. They were bottom-watered with tap water, twice weekly. Twenty-four days old plants were sprayed with either water (control) or 0.6% ammonium nitrate at 4 μL/cm² of tray surface. Total shoots and some primary roots were cleaned of vermiculite, flash-frozen in liquid nitrogen and stored at −80° C.

(h) Methyl Jasmonate

Seeds of Arabidopsis thaliana (ecotype Wassilewskija) were sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants were spayed with 200-250 mls of 0.001% methyl jasmonate in a 0.02% solution of the detergent Silwet L-77. At 1 hr and 6 hrs after treatment, whole seedlings, including roots, were harvested within a 15 to 20 minute time period, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 0.001% methyl jasmonate for treatment. Control plants were treated with water. After 24 hr, aerial and root tissues were separated and flash frozen in liquid nitrogen prior to storage at −80° C.

(i) Salicylic Acid

Seeds of Arabidopsis thaliana (ecotype Wassilewskija) were sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants were spayed with 200-250 mls of 5 mM salicylic acid (solubilized in 70% ethanol) in a 0.02% solution of the detergent Silwet L-77. At 1 hr and 6 hrs after treatment, whole seedlings, including roots, were harvested within a 15 to 20 minute time period flash-frozen in liquid nitrogen and stored at −80° C.

Alternatively, seeds of wild-type Arabidopsis thaliana (ecotype Columbia) and mutant CS3726 were sown in soil type 200 mixed with osmocote fertilizer and Marathon insecticide and left at 4° C. for 3 days to vernalize. Flats were incubated at room temperature with continuous light. Sixteen days post germination plants were sprayed with 2 mM SA, 0.02% SilwettL-77 or control solution (0.02% SilwettL-77. Aerial parts or flowers were harvested 1 hr, 4 hr, 6 hr, 24 hr and 3 weeks post-treatment flash frozen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 2 mM SA for treatment. Control plants were treated with water. After 12 hr and 24 hr, aerial and root tissues were separated and flash frozen in liquid nitrogen prior to storage at −80° C.

(j) Drought Stress

Seeds of Arabidopsis thaliana (Wassilewskija) were sown in pots and left at 4° C. for three days to vernalize before being transferred to a growth chamber having 16 hr light/8 hr dark, 150,000-160,000 LUX, 20° C. and 70% humidity. After 14 days, aerial tissues were cut and left to dry on 3 MM Whatman paper in a petri-plate for 1 hour and 6 hours. Aerial tissues exposed for 1 hour and 6 hours to 3 MM Whatman paper wetted with 1× Hoagland's solution served as controls. Tissues were harvested, flash-frozen in liquid nitrogen and stored at −80° C.

Alternatively, Arabidopsis thaliana (Ws) seed was vernalized at 4° C. for 3 days before sowing in Metromix soil type 350. Flats were placed in a growth chamber with 23° C., 16 hr light/8 hr. dark, 80% relative humidity, ˜13,000 LUX for germination and growth. Plants were watered with 1-1.5 L of water every four days. Watering was stopped 16 days after germination for the treated samples, but continued for the control samples. Rosette leaves and stems, flowers and siliques were harvested 2 d, 3 d, 4 d, 5 d, 6 d and 7 d after watering was stopped. Tissue was flash frozen in liquid nitrogen and kept at −80° C. until RNA was isolated. Flowers and siliques were also harvested on day 8 from plants that had undergone a 7 d drought treatment followed by 1 day of watering. Control plants (whole plants) were harvested after 5 weeks, flash frozen in liquid nitrogen and stored as above.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in empty 1-liter beakers at room temperature for treatment. Control plants were placed in water. After 1 hr, 6 hr, 12 hr and 24 hr aerial and root tissues were separated and flash frozen in liquid nitrogen prior to storage at −80° C.

(k) Osmotic Stress

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C., and 70% humidity. After 14 days, the aerial tissues were cut and placed on 3 MM Whatman paper in a petri-plate wetted with 20% PEG (polyethylene glycol-Mᵣ 8,000) in 1× Hoagland's solution. Aerial tissues on 3 MM Whatman paper containing 1× Hoagland's solution alone served as the control. Aerial tissues were harvested at 1 hour and 6 hours after treatment, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 10% PEG (polyethylene glycol-Mᵣ 8,000) for treatment. Control plants were treated with water. After 1 hr and 6 hr aerial and root tissues were separated and flash frozen in liquid nitrogen prior to storage at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 150 mM NaCl for treatment. Control plants were treated with water. After 1 hr, 6 hr, and 24 hr aerial and root tissues were separated and flash frozen in liquid nitrogen prior to storage at −80° C.

(1) Heat Shock Treatment

Seeds of Arabidopsis Thaliana (Wassilewskija) were sown in trays and left at 4° C. for three days to vernalize before being transferred to a growth chamber with 16 hr light/8 hr dark, 12,000-14,000 Lux, 70% humidity and 20° C., fourteen day old plants were transferred to a 42° C. growth chamber and aerial tissues were harvested 1 hr and 6 hr after transfer. Control plants were left at 20° C. and aerial tissues were harvested. Tissues were flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers containing 42° C. water for treatment. Control plants were treated with water at 25° C. After 1 hr and 6 hr aerial and root tissues were separated and flash frozen in liquid nitrogen prior to storage at −80° C.

(m) Cold Shock Treatment

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C. and 70% humidity. Fourteen day old plants were transferred to a 4° C. dark growth chamber and aerial tissues were harvested 1 hour and 6 hours later. Control plants were maintained at 20° C. and covered with foil to avoid exposure to light. Tissues were flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers containing 4° C. water for treatment. Control plants were treated with water at 25° C. After 1 hr and 6 hr aerial and root tissues were separated and flash frozen in liquid nitrogen prior to storage at −80° C.

(n) Arabidopsis Seeds

Fruits (Pod+Seed) 0-5 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Siliques 0-5 mm in length containing post fertilization through pre-heart stage [0-72 hours after fertilization (HAF)] embryos were harvested and flash frozen in liquid nitrogen.

Fruits (Pod+Seed) 5-10 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Siliques 5-10 mm in length containing heart—through early upturned-U—stage [72-120 hours after fertilization (HAF)] embryos were harvested and flash frozen in liquid nitrogen.

Fruits (Pod+Seed)>10 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Siliques >10 mm in length containing green, late upturned-U—stage [>120 hours after fertilization (HAF)-9 days after flowering (DAF)] embryos were harvested and flash frozen in liquid nitrogen.

Green Pods 5-10 mm (Control Tissue for Samples 72-74)

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques 5-10 mm in length containing developing seeds 72-120 hours after fertilization (HAF)] were opened and the seeds removed. The remaining tissues (green pods minus seed) were harvested and flash frozen in liquid nitrogen.

Green Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques >10 mm in length containing developing seeds up to 9 days after flowering (DAF)] were opened and the seeds removed and harvested and flash frozen in liquid nitrogen.

Brown Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Yellowing siliques >10 mm in length containing brown, dessicating seeds >11 days after flowering (DAF)] were opened and the seeds removed and harvested and flash frozen in liquid nitrogen.

Green/Brown Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques >10 mm in length containing both green and brown seeds >9 days after flowering (DAF)] were opened and the seeds removed and harvested and flash frozen in liquid nitrogen.

Mature Seeds (24 Hours after Imbibition)

Mature dry seeds of Arabidopsis thaliana (ecotype Wassilewskija) were sown onto moistened filter paper and left at 4° C. for two to three days to vernalize. Imbibed seeds were then transferred to a growth chamber [16 hr light: 8 hr dark conditions, 7000-8000 LUX light intensity, 70% humidity, and 22° C. temperature], the emerging seedlings harvested after 48 hours and flash frozen in liquid nitrogen.

Mature Seeds (Dry)

Seeds of Arabidopsis thaliana (ecotype Wassilewskija) were sown in pots and left at 4° C. for two to three days to vernalize. They were then transferred to a growth chamber. Plants were grown under long-day (16 hr light: 8 hr dark) conditions, 7000-8000 LUX light intensity, 70% humidity, and 22° C. temperature and taken to maturity. Mature dry seeds are collected, dried for one week at 28° C., and vernalized for one week at 4° C. before used as a source of RNA.

(o) Herbicide Treament

Arabidopsis thaliana (Ws) seeds were sterilized for 5 min. with 30% bleach, 50 μl Triton in a total volume of 50 ml. Seeds were vernalized at 4° C. for 3 days before being plated onto GM agar plates at a density of about 144 seeds per plate. Plates were incubated in a Percival growth chamber having 16 hr light/8 hr dark, 80% relative humidity, 22° C. and 11,000 LUX for 14 days.

Plates were sprayed (˜0.5 mls/plate) with water, Finale (1.128 g/L), Glean (1.88 g/L), RoundUp (0.01 g/L) or Trimec (0.08 g/L). Tissue was collected and flash frozen in liquid nitrogen at the following time points: 0, 1, 2, 4, 8, 12 and 24 hours. Frozen tissue was stored at −80° C. prior to RNA isolation.

(p) Root Tips

Seeds of Arabidopsis thaliana (ecotye Ws) were placed on MS plates and vernalized at 4° C. for 3 days before being placed in a 25° C. growth chamber having 16 hr light/8 hr dark, 70% relative humidty and about 3 W/m². After 6 days, young seedlings were transferred to flasks containing B5 liquid medium, 1% sucrose and 0.05 mg/l indole-3-butyric acid. Flasks were incubated at room temperature with 100 rpm agitation. Media was replaced weekly. After three weeks, roots were harvested and incubated for 1 hr with 2% pectinase, 0.2% cellulase, pH 7 before straining through a #80 (Sigma) sieve. The root body material remaining on the sieve (used as the control) was flash frozen and stored at −80° C. until use. The material that passed through the #80 sieve was strained through a #200 (Sigma) sieve and the material remaining on the sieve (root tips) was flash frozen and stored at −80° C. until use. Approximately 10 mg of root tips were collected from one flask of root culture.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 8 days. Seedlings were carefully removed from the sand and the root tips (˜2 mm long) were removed and flash frozen in liquid nitrogen prior to storage at −80° C. The tissues above the root tips (˜1 cm long) were cut, treated as above and used as control tissue.

(q) Imbibed Seed

Seeds of maize hybrid 35A (Pioneer) were sown in water-moistened sand in covered flats (10 rows, 5-6 seed/row) and covered with clear, plastic lids before being placed in a growth chamber having 16 hr light (25° C.)/8 hr dark (20° C.), 75% relative humidity and 13,000-14,000 LUX. One day after sowing, whole seeds were flash frozen in liquid nitrogen prior to storage at −80° C. Two days after sowing, embryos and endosperm were isolated and flash frozen in liquid nitrogen prior to storage at −80° C. On days 3-6, aerial tissues, roots and endosperm were isolated and flash frozen in liquid nitrogen prior to storage at −80° C.

(r) Flowers (Green, White or Buds)

Approximately 10 □l of Arabidopsis thaliana seeds (ecotype Ws) were sown on 350 soil (containing 0.03% marathon) and vernalized at 4 C for 3 days. Plants were then grown at room temperature under fluorescent lighting until flowering. Flowers were harvested after 28 days in three different categories. Buds that had not opened at all and were completely green were categorized as “flower buds” (also referred to as green buds by the investigator). Buds that had started to open, with white petals emerging slightly were categorized as “green flowers” (also referred to as white buds by the investigator). Flowers that had opened mostly (with no silique elongation) with white petals completely visible were categorized as “white flowers” (also referred to as open flowers by the investigator). Buds and flowers were harvested with forceps, flash frozen in liquid nitrogen and stored at −80 C until RNA was isolated.

s) Ovules

Seeds of Arabidopsis thaliana heterozygous for pistillata (pi) [ecotype Landsberg erecta (Ler)] were sown in pots and left at 4° C. for two to three days to vernalize. They were then transferred to a growth chamber. Plants were grown under long-day (16 hr light: 8 hr dark) conditions, 7000-8000 LUX light intensity, 76% humidity, and 24° C. temperature. Inflorescences were harvested from seedlings about 40 days old. The inflorescences were cut into small pieces and incubated in the following enzyme solution (pH 5) at room temperature for 0.5-1 hr.: 0.2% pectolyase Y-23, 0.04% pectinase, 5 mM MES, 3% Sucrose and MS salts (1900 mg/l KNO₃, 1650 mg/l NH₄NO₃, 370 mg/l MgSO₄.7H₂O, 170 mg/l KH₂PO₄, 440 mgA CaCl₂.2H₂O, 6.2 mg/l H₂BO₃, 15.6 mgA MnSO₄.4H₂O, 8.6 mg/l ZnSO₄.7H₂O, 0.25 mg/l NaMoO₄.2H₂O, 0.025 mg/l CuCO₄.5H₂O, 0.025 mg/l CoCl₂.6H₂O, 0.83 mg/l KI, 27.8 mg/l FeSO₄.7H₂O, 37.3 mg/l Disodium EDTA, pH 5.8). At the end of the incubation the mixture of inflorescence material and enzyme solution was passed through a size 60 sieve and then through a sieve with a pore size of 125 μm. Ovules greater than 125 μm in diameter were collected, rinsed twice in B5 liquid medium (2500 mg/l KNO_{3,} 250 mg/l MgSO₄.7H₂O, 150 mg/l NaH2PO4.H₂O, 150 mg/l CaCl₂.2H₂O, 134 mg/l (NH4)2 CaCl₂.SO₄, 3 mg/l H₂BO₃, 10 mg/l MnSO₄.4H₂O, 2 ZnSO₄.7H₂O, 0.25 mg/l NaMoO₄.2H₂O, 0.025 mg/l CuCO₄. 5H₂O, 0.025 mg/l CoCl₂.6H₂O, 0.75 mg/l KI, 40 mg/l EDTA sodium ferric salt, 20 g/l sucrose, 10 mg/l Thiamine hydrochloride, 1 mg/l Pyridoxine hydrochloride, 1 mg/l Nicotinic acid, 100 mg/l myo-inositol, pH 5.5)), rinsed once in deionized water and flash frozen in liquid nitrogen. The supernatant from the 125 μm sieving was passed through subsequent sieves of 50 μm and 32 μm. The tissue retained in the 32 μm sieve was collected and mRNA prepared for use as a control.

t) Wounding

Seeds of Arabidopsis thaliana (Wassilewskija) were sown in trays and left at 4° C. for three days to vernalize before being transferred to a growth chamber having 16 hr light/8 hr dark, 12,000-14,000 LUX, 70% humidity and 20° C. After 14 days, the leaves were wounded with forceps. Aerial tissues were harvested 1 hour and 6 hours after wounding. Aerial tissues from unwounded plants served as controls. Tissues were flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were wounded (one leaf nicked by scissors) and placed in 1-liter beakers of water for treatment. Control plants were treated not wounded. After 1 hr and 6 hr aerial and root tissues were separated and flash frozen in liquid nitrogen prior to storage at −80° C.

u) Nitric Oxide Treatment

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C. and 70% humidity. Fourteen day old plants were sprayed with 5 mM sodium nitroprusside in a 0.02% Silwett L-77 solution. Control plants were sprayed with a 0.02% Silwett L-77 solution. Aerial tissues were harvested 1 hour and 6 hours after spraying, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 5 mM nitroprusside for treatment. Control plants were treated with water. After 1 hr, 6 hr and 12 hr, aerial and root tissues were separated and flash frozen in liquid nitrogen prior to storage at −80° C.

v) Root Hairless Mutants

Plants mutant at the rhl gene locus lack root hairs. This mutation is maintained as a heterozygote.

Seeds of Arabidopsis thaliana (Landsberg erecta) mutated at the rhl gene locus were sterilized using 30% bleach with 1 ul/ml 20% Triton-X 100 and then vernalized at 4° C. for 3 days before being plated onto GM agar plates. Plates were placed in growth chamber with 16 hr light/8 hr. dark, 23° C., 14,500-15,900 LUX, and 70% relative humidity for germination and growth.

After 7 days, seedlings were inspected for root hairs using a dissecting microscope. Mutants were harvested and the cotyledons removed so that only root tissue remained. Tissue was then flash frozen in liquid nitrogen and stored at −80 C.

Arabidopsis thaliana (Landsberg erecta) seedlings grown and prepared as above were used as controls.

Alternatively, seeds of Arabidopsis thaliana (Landsberg erecta), heterozygous for the rhl1 (root hairless) mutation, were surface-sterilized in 30% bleach containing 0.1% Triton X-100 and further rinsed in sterile water. They were then vernalized at 4° C. for 4 days before being plated onto MS agar plates. The plates were maintained in a growth chamber at 24° C. with 16 hr light/8 hr dark for germination and growth. After 10 days, seedling roots that expressed the phenotype (i.e. lacking root hairs) were cut below the hypocotyl junction, frozen in liquid nitrogen and stored at −80° C. Those seedlings with the normal root phenotype (heterozygous or wt) were collected as described for the mutant and used as controls.

w) Ap2

Seeds of Arabidopsis thaliana (ecotype Landesberg erecta) and floral mutant apetala2 (Jofuku et al., 1994, Plant Cell 6:1211-1225) were sown in pots and left at 4° C. for two to three days to vernalize. They were then transferred to a growth chamber. Plants were grown under long-day (16 hr light, 8 hr dark) conditions 7000-8000 LUX light intensity, 70% humidity and 22° C. temperature. Inflorescences containing immature floral buds (stages 1-7; Bowman, 1994) as well as the inflorescence meristem were harvested and flash frozen. Polysomal polyA+ RNA was isolated from tissue according to Cox and Goldberg, 1988).

x) Salt

Arabidopsis thaliana ecotype Ws seeds were vernalized at 4° C. for 3 days before sowing in flats containing vermiculite soil. Flats were placed at 20° C. in a Conviron growth chamber having 16 hr light/8 hr dark. Whole plants (used as controls) received water. Other plants were treated with 100 mM NaCl. After 6 hr and 72 hr, aerial and root tissues were harvested and flash frozen in liquid nitrogen prior to storage at −80° C.

y) Petals

Arabidopsis thaliana ecotype Ws seeds were vernalized at 4° C. for 3 days before sowing in flats containing vermiculite soil. Flats were watered placed at 20° C. in a Conviron growth chamber having 16 hr light/8 hr dark. Whole plants (used as the control) and petals from inflorescences 23-25 days after germination were harvested, flash frozen in liquid nitrogen and stored at −80° C.

z) Pollen

Arabidopsis thaliana ecotype Ws seeds were vernalized at 4° C. for 3 days before sowing in flats containing vermiculite soil. Flats were watered and placed at 20° C. in a Conviron growth chamber having 16 hr light/8 hr dark. Whole plants (used as controls) and pollen from plants 38 dap was harvested, flash frozen in liquid nitrogen and stored at −80° C.

aa) Interploidy Crosses

Interploidy crosses involving a 6× parent are lethal. Crosses involving a 4× parent are compelte and analyzed. The imbalance in the maternal/paternal ratio produced from the cross can lead to big seeds. Arabidopsis thaliana ecotype Ws seeds were vernalized at 4° C. for 3 days before sowing. Small siliques were harvested at 5 days after pollination, flash frozen in liquid nitrogen and stored at −80° C.

bb) Line Comparisons

Alkaloid 35S over-expressing lines were used to monitor the expression levels of terpenoid/alkaloid biosynthetic and P450 genes to identify the transcriptional regulatory points in the biosynthesis pathway and the related P450 genes. Arabidopsis thaliana ecotype Ws seeds were vernalized at 4° C. for 3 days before sowing in vermiculite soil (Zonolite) supplemented by Hoagland solution. Flats were placed in Conviron growth chambers under long day conditions (16 hr light, 23° C./8 hr dark, 20° C.) Basta spray and selection of the overexpressing lines was conducted about 2 weeks after germination. Approximately 2-3 weeks after bolting (approximately 5-6 weeks after germination), aerial portions (e.g. stem and siliques) from the overexpressing lines and from wild-type plants were harvested, flash frozen in liquid nitrogen and stored at −80° C.

cc) DMT-II

Demeter (dmt) is a mutant of a methyl transferase gene and is similar to fie. Arabidopsis thaliana ecotype Ws seeds were vernalized at 4° C. for 3 days before sowing. Cauline leaves and closed flowers were isolated from 35S::DMT and dmt −/− plant lines, flash frozen in liquid nitrogen and stored at −80° C.

dd) CS6630 Roots and Shoots

Arabidopsis thaliana ecotype Ws seeds were vernalized at 4° C. for 3 days before sowing on MS media (1%) sucrose on bactor-agar. Roots and shoots were separated 14 days after germination, flash frozen in liquid nitrogen and stored at −80° C.

ee) CS237

CS237 is an ethylene triple response mutant that is insensitive to ethylene and which has an etr1-1 phenotype. Arabidopsis thaliana CS237 seeds were vernalized at 4° C. for 3 days before sowing. Aerial tissue was collected from mutants and wild-type Columbia ecotype plants, flash frozen in liquid nitrogen and stored at −80° C.

ff) Guard Cells

Arabidopsis thaliana ecotype Ws seeds were vernalized at 4° C. for 3 days before sowing. Leaves were harvested, homogenized and centrifuged to isolate the guard cell containing fraction. Homogenate from leaves served as the control. Samples were flash frozen in liquid nitrogen and stored at −80° C. Identical experiments using leaf tissue from canola were performed.

gg) 3642-1

3642-1 is a T-DNA mutant that affects leaf development. This mutant segregates 3:1, wild-type:mutant. Arabidopsis thaliana 3642-1 mutant seeds were vernalized at 4° C. for 3 days before sowing in flats of MetroMix 200. Flats were placed in the greenhouse, watered and grown to the 8 leaf, pre-flower stage. Stems and rosette leaves were harvested from the mutants and the wild-type segregants, flash frozen and stored at −80° C.

hh) Caf

Carple factory (Caf) is a double-stranded RNAse protein that is hypothesized to process small RNAs in Arabidopsis. The protein is closely related to a Drosophila protein named DICER that functions in the RNA degradation steps of RNA interference. Arabidopsis thaliana Caf mutant seeds were vernalized at 4° C. for 3 days before sowing in flats of MetroMix 200. Flats were placed in the greenhouse, watered and grown to the 8 leaf, pre-flower stage. Stems and rosette leaves were harvested from the mutants and the wild-type segregants, flash frozen and stored at −80° C.

2. Microarray Hybridization Procedures

Microarray technology provides the ability to monitor mRNA transcript levels of thousands of genes in a single experiment. These experiments simultaneously hybridize two differentially labeled fluorescent cDNA pools to glass slides that have been previously spotted with cDNA clones of the same species. Each arrayed cDNA spot will have a corresponding ratio of fluorescence that represents the level of disparity between the respective mRNA species in the two sample pools. Thousands of polynucleotides can be spotted on one slide, and each experiment generates a global expression pattern.

Coating Slides

The microarray consists of a chemically coated microscope slide, referred herein as a “chip” with numerous polynucleotide samples arrayed at a high density. The poly-L-lysine coating allows for this spotting at high density by providing a hydrophobic surface, reducing the spreading of spots of DNA solution arrayed on the slides. Glass microscope slides (Gold Seal #3010 manufactured by Gold Seal Products, Portsmouth, N.H., USA) were coated with a 0.1% WN solution of Poly-L-lysine (Sigma, St. Louis, Mo.) using the following protocol:
1. Slides were placed in slide racks (Shandon Lipshaw #121). The racks were then put in chambers (Shandon Lipshaw #121).
2. Cleaning solution was prepared:
   70 g NaOH was dissolved in 280 nL ddH₂O.
   420 mL 95% ethanol was added. The total volume was 700 mL (=2×350 mL); it was stirred until completely mixed. If the solution remained cloudy, ddH₂O was added until clear.
3. The solution was poured into chambers with slides; the chambers were covered with glass lids. The solution was mixed on an orbital shaker for 2 hr.
4. The racks were quickly transferred to fresh chambers filled with ddH₂O. They were rinsed vigorously by plunging racks up and down. Rinses were repeated 4× with fresh ddH₂O each time, to remove all traces of NaOH-ethanol.
5. Polylysine solution was prepared:
   0 mL poly-L-lysine+70 mL tissue culture PBS in 560 mL water, using plastic graduated cylinder and beaker.
6. Slides were transferred to polylysine solution and shaken for 1 hr.
7. The rack was transferred to a fresh chambers filled with ddH₂O. It was plunged up and down 5× to rinse.
8. The slides were centrifuged on microtiter plate carriers (paper towels were placed below the rack to absorb liquid) for 5 min. @ 500 rpm. The slide racks were transferred to empty chambers with covers.
9. Slide racks were dried in a 45 C oven for 10 min.
10. The slides were stored in a closed plastic slide box.
11. Normally, the surface of lysine coated slides was not very hydrophobic immediately after this process, but became increasingly hydrophobic with storage. A hydrophobic surface helped ensure that spots didn't run together while printing at high densities. After they aged for 10 days to a month the slides were ready to use. However, coated slides that have been sitting around for long periods of time were usually too old to be used. This was because they developed opaque patches, visible when held to the light, and these resulted in high background hybridization from the fluorescent probe. Alternatively, pre-coated glass slides were purchased from TeleChem International, Inc. (Sunnyvale, Calif., 94089; catalog number SMM-25, Superamine substrates).
   PCR Amplification of cDNA Clone Inserts

Polynucleotides were amplified from Arabidopsis cDNA clones using insert specific probes. The resulting 100 uL PCR reactions were purified with Qiaquick 96 PCR purification columns (Qiagen, Valencia, Calif., USA) and eluted in 30 uL of 5 mM Tris. 8.5 uL of the elution were mixed with 1.5 uL of 20×SSC to give a final spotting solution of DNA in 3×SSC. The concentrations of DNA generated from each clone varied between 10-100 ng/ul, but were usually about 50 ng/ul.

Arraying of PCR Products on Glass Slides

PCR products from cDNA clones were spotted onto the poly-L-Lysine coated glass slides using an arrangement of quill-tip pins (ChipMaker 3 spotting pins; Telechem, International, Inc., Sunnyvale, Calif., USA) and a robotic arrayer (PixSys 3500, Cartesian Technologies, Irvine, Calif., USA). Around 0.5 nl of a prepared PCR product was spotted at each location to produce spots with approximately 100 um diameters. Spot center-to-center spacing was from 180 um to 210 um depending on the array. Printing was conducted in a chamber with relative humidity set at 50%.

Slides containing maize sequences were purchased from Agilent Technology (Palo Alto, Calif. 94304).

Post-Processing of Slides

After arraying, slides were processed through a series of steps—rehydration, UV cross-linking, blocking and denaturation—required prior to hybridization. Slides were rehydrated by placing them over a beaker of warm water (DNA face down), for 2-3 sec, to distribute the DNA more evenly within the spots, and then snap dried on a hot plate (DNA side, face up). The DNA was then cross-linked to the slides by UV irradiation (60-65 mJ; 2400 Stratalinker, Stratagene, La Jolla, Calif., USA).

Following this a blocking step was performed to modify remaining free lysine groups, and hence minimize their ability to bind labeled probe DNA. To achieve this the arrays were placed in a slide rack. An empty slide chamber was left ready on an orbital shaker. The rack was bent slightly inwards in the middle, to ensure the slides would not run into each other while shaking. The blocking solution was prepared as follows:
3×350-ml glass chambers (with metal tops) were set to one side, and a large round Pyrex dish with dH₂O was placed ready in the microwave. At this time, 15 ml sodium borate was prepared in a 50 ml conical tube.

6-g succinic anhydride was dissolved in approx. 325-350 mL 1-methyl-2-pyrrolidinone. Rapid addition of reagent was crucial.

a. Immediately after the last flake of the succinic anhydride dissolved, the 15-mL sodium borate was added.

b. Immediately after the sodium borate solution mixed in, the solution was poured into an empty slide chamber.

c. The slide rack was plunged rapidly and evenly in the solution. It was vigorously shaken up and down for a few seconds, making sure slides never left the solution.

d. It was mixed on an orbital shaker for 15-20 min. Meanwhile, the water in the Pyrex dish (enough to cover slide rack) was heated to boiling.

Following this, the slide rack was gently plunge in the 95 C water Oust stopped boiling) for 2 min. Then the slide rack was plunged 5× in 95% ethanol. The slides and rack were centrifuged for 5 min. @ 500 rpm. The slides were loaded quickly and evenly onto the carriers to avoid streaking. The arrays were used immediately or store in slide box.

The Hybridization process began with the isolation of mRNA from the two tissues (see “Isolation of total RNA” and “Isolation of mRNA”, below) in question followed by their conversion to single stranded cDNA (see “Generation of probes for hybridization”, below). The cDNA from each tissue was independently labeled with a different fluorescent dye and then both samples were pooled together. This final differentially labeled cDNA pool was then placed on a processed microarray and allowed to hybridize (see “Hybridization and wash conditions”, below).

Isolation of Total RNA

Approximately 1 g of plant tissue was ground in liquid nitrogen to a fine powder and transferred into a 50-ml centrifuge tube containing 10 ml of Trizol reagent. The tube was vigorously vortexed for 1 nin and then incubated at room temperature for 10-20 min. on an orbital shaker at 220 rpm. Two ml of chloroform was added to the tube and the solution vortexed vigorously for at least 30-sec before again incubating at room temperature with shaking. The sample was then centrifuged at 12,000×g (10,000 rpm) for 15-20 min at 4° C. The aqueous layer was removed and mixed by inversion with 2.5 ml of 1.2 M NaCl/0.8 M Sodium Citrate and 2.5 ml of isopropyl alcohol added. After a 10 min. incubation at room temperature, the sample was centrifuged at 12,000×g (10,000 rpm) for 15 min at 4° C. The pellet was washed with 70% ethanol, re-centrifuged at 8,000 rpm for 5 min and then air dried at room temperature for 10 min. The resulting total RNA was dissolved in either TE (10 mM Tris-HCl, 1 mM EDTA, pH 8.0) or DEPC (diethylpyrocarbonate) treated deionized water (RNAse-free water). For subsequent isolation of mRNA using the Qiagen kit, the total RNA pellet was dissolved in RNAse-free water.

Isolation of mRNA

mRNA was isolated using the Qiagen Oligotex mRNA Spin-Column protocol (Qiagen, Valencia, Calif.). Briefly, 500 μl OBB buffer (20 mM Tris-Cl, pH 7.5, 1 M NaCl, 2 mM EDTA, 0.2% SDS) was added to 500 μl of total RNA (0.5-0.75 mg) and mixed thoroughly. The sample was first incubated at 70° C. for 3 min, then at room temperature for 10 minutes and finally centrifuged for 2 min at 14,000-18,000×g. The pellet was resuspended in 400 μl OW2 buffer (10 mM Tris-Cl, pH 7.5, 150 mM NaCl, 1 mM EDTA) by vortexing, the resulting solution placed on a small spin column in a 1.5 ml RNase-free microcentrifuge tube and centrifuged for 1 min at 14,000-18,000×g. The spin column was transferred to a new 1.5 ml RNase-free microcentrifuge tube and washed with 400 μl of OW2 buffer. To release the isolated mRNA from the resin, the spin column was again transferred to a new RNase-free 1.5 ml microcentrifuge tube, 20-100 μl 70° C. OEB buffer (5 mM Tris-Cl, pH 7.5) added and the resin resuspended in the resulting solution via pipeting. The mRNA solution was collected after centrifuging for 1 min at 14,000-18,000×g.

Alternatively, mRNA was isolated using the Stratagene Poly(A) Quik mRNA Isolation Kit (Startagene, La Jolla, Calif.). Here, up to 0.5 mg of total RNA (maximum volume of 1 ml) was incubated at 65° C. for 5 minutes, snap cooled on ice and 0.1× volumes of 10× sample buffer (10 mM Tris-HCl (pH 7.5), 1 mM EDTA (pH 8.0) 5 M NaCl) added. The RNA sample was applied to a prepared push column and passed through the column at a rate of ˜1 drop every 2 sec. The solution collected was reapplied to the column and collected as above. 200 μl of high salt buffer (10 mM Tris-HCl (pH 7.5), 1 mM EDTA, 0.5 NaCl) was applied to the column and passed through the column at a rate of ˜1 drop every 2 sec. This step was repeated and followed by three low salt buffer (10 mM Tris-HCl (pH 7.5), 1 mM EDTA, 0.1 M NaCl) washes preformed in a similar manner. mRNA was eluted by applying to the column four separate 200 μl aliquots of elution buffer (10 mM Tris-HCl (pH 7.5), 1 mM EDTA) preheated to 65° C. Here, the elution buffer was passed through the column at a rate of 1 drop/sec. The resulting mRNA solution was precipitated by adding 0.1× volumes of 10× sample buffer, 2,5 volumes of ice-cold 100% ethanol, incubating overnight at −20° C. and centrifuging at 14,000-18,000×g for 20-30 min at 4° C. The pellet was washed with 70% ethanol and air dried for 10 min. at room temperature before resuspension in RNase-free deionized water.

Preparation of Yeast Controls

Plasmid DNA was isolated from the following yeast clones using Qiagen filtered maxiprep kits (Qiagen, Valencia, Calif.): YAL022c(Fun26), YAL031c(Fun21), YBR032w, YDL131w, YDL182w, YDL194w, YDL196w, YDR050c and YDR116c. Plasmid DNA was linearized with either BsrBI (YAL022c(Fun26), YAL031c(Fun21), YDL131w, YDL182w, YDL194w, YDL196w, YDR050c) or AflIII (YBR032w, YDR116c) and isolated.

In Vitro Transcription of Yeast Clones

The following solution was incubated at 37° C. for 2 hours: 17 μl of isolated yeast insert DNA (1 μg), 20 μl 5× buffer, 10 μl 100 mM DTT, 2.5 μl (100 U) RNasin, 20 μl 2.5 mM (ea.) rNTPs, 2.7 μl (40U) SP6 polymerase and 27.8 μl RNase-free deionized water. 2 μl (2 U) Ampli DNase I was added and the incubation continued for another 15 min. 10 μl SM NH₄OAC and 100 μl phenol:chloroform:isoamyl alcohol (25:24:1) were added, the solution vortexed and then centrifuged to separate the phases. To precipitate the RNA, 250 μl ethanol was added and the solution incubated at −20° C. for at least one hour. The sample was then centrifuged for 20 min at 4° C. at 14,000-18,000×g, the pellet washed with 500 μl of 70% ethanol, air dried at room temperature for 10 min and resuspended in 100 μl of RNase-free deionized water. The precipitation procedure was then repeated.

Alternatively, after the two-hour incubation, the solution was extracted with phenol/chloroform once before adding 0.1 volume 3M sodium acetate and 2.5 volumes of 100% ethanol. The solution was centrifuged at 15,000 rpm, 4° C. for 20 minutes and the pellet resuspended in RNase-free deionized water. The DNase I treatment was carried out at 37° C. for 30 minutes using 2 U of Ampli DNase I in the following reaction condition: 50 mM Tris-HCl (pH 7.5), 10 mM MgCl₂. The DNase I reaction was then stopped with the addition of NH₄OAC and phenol:chloroform:isoamyl alcohol (25:24:1), and RNA isolated as described above. 0.15-2.5 ng of the in vitro transcript RNA from each yeast clone were added to each plant mRNA sample prior to labeling to serve as positive (internal) probe controls.

Generation of Probes for Hybridization

Generation of Labeled Probes for Hybridization from First-Strand cDNA

Hybridization probes were generated from isolated mRNA using an Atlas™ Glass Fluorescent Labeling Kit (Clontech Laboratories, Inc., Palo Alto, Calif., USA). This entails a two step labeling procedure that first incorporates primary aliphatic amino groups during cDNA synthesis and then couples fluorescent dye to the cDNA by reaction with the amino functional groups. Briefly, 5 μg of oligo(dT)₁₈ primer d(TTTTTTTTTTTTTTTTTTV) was mixed with Poly A+ mRNA (1.5-2 μg mRNA isolated using the Qiagen Oligotex mRNA Spin-Column protocol or the Stratagene Poly(A) Quik mRNA Isolation protocol (Stratagene, La Jolla, Calif., USA)) in a total volume of 25 μl. The sample was incubated in a thermocycler at 70° C. for 5 min, cooled to 48° C. and 10 μl of 5× cDNA Synthesis Buffer (kit supplied), 5 μl 10× DNTP mix (DATP, dCTP, dGTP, dTTP and aminoallyl-dUTP; kit supplied), 7.5 μl deionized water and 2.5 μl MMLV Reverse Transcriptase (500U) added. The reaction was then incubated at 48° C. for 30 minutes, followed by 1 hr incubation at 42° C. At the end of the incubation the reaction was heated to 70° C. for 10 min, cooled to 37° C. and 0.5 μl (5 U) RNase H added, before incubating for 15 min at 37° C. The solution was vortexed for 1 min after the addition of 0.5 μl 0.5 M EDTA and 5 μl of QuickClean Resin (kit supplied) then centrifuged at 14,000-18,000×g for 1 min. After removing the supernatant to a 0.45 μm spin filter (kit supplied), the sample was again centrifuged at 14,000-18,000×g for 1 min, and 5.5 μl 3 M sodium acetate and 137.5 μl of 100% ethanol added to the sample before incubating at −20° C. for at least 1 hr. The sample was then centrifuged at 14,000-18,000×g at 4° C. for 20 min, the resulting pellet washed with 500 μl 70% ethanol, air-dried at room temperature for 10 min and resuspended in 10 μl of 2× fluorescent labeling buffer (kit provided). 10 μl each of the fluorescent dyes Cy3 and Cy5 (Amersham Pharmacia (Piscataway, N.J., USA); prepared according to Atlas™ kit directions of Clontech) were added and the sample incubated in the dark at room temperature for 30 min.

The fluorescently labeled first strand cDNA was precipitated by adding 2 μl 3M sodium acetate and 50 μl 100% ethanol, incubated at −20° C. for at least 2 hrs, centrifuged at 14,000-18,000×g for 20 min, washed with 70% ethanol, air-dried for 10 min and dissolved in 100 μl of water.

Alternatively, 3-4 μg mRNA, 2.5 (˜8.9 ng of in vitro translated mRNA) μl yeast control and 3 μg oligo dTV (TTTTTTTTTTTTTTTTTT(A/C/G) were mixed in a total volume of 24.7 μl. The sample was incubated in a thermocycler at 70° C. for 10 min. before chilling on ice. To this, 8 μl of 5× first strand buffer (SuperScript II RNase H—Reverse Transcriptase kit from Invitrogen (Carlsbad, Calif. 92008); cat no. 18064022), 0.8° C. of aa-dUTP/dNTP mix (50×; 25 mM dATP, 25 mM dGTP, 25 mM dCTP, 15 mM dTTP, 10 mM aminoallyl-dUTP), 4 μl of 0.1 M DTT and 2.5 μl (500 units) of Superscript R.T.II enzyme (Stratagene) were added. The sample was incubated at 42° C. for 2 hours before a mixture of 10° C. of 1 M NaOH and 10° C. of 0.5 M EDTA were added. After a 15 minute incubation at 65° C., 25 μl of 1 M Tris pH 7.4 was added. This was mixed with 450 μl of water in a Microcon 30 column before centrifugation at 11,000×g for 12 min. The column was washed twice with 450 μl (centrifugation at 11,000 g, 12 min.) before eluting the sample by inverting the Microcon column and centrifuging at 11,000×g for 20 seconds. Sample was dehydrated by centrifugation under vacuum and stored at −20° C.

Each reaction pellet was dissolved in 9 μl of 0.1 M carbonate buffer (0.1 M sodium carbonate and sodium bicarbonate, pH=8.5-9) and 4.5 μl of this placed in two microfuge tubes. 4.5 μl of each dye (in DMSO) were added and the mixture incubated in the dark for 1 hour. 4.5 μl of 4 M hydroxylamine was added and again incubated in the dark for 15 minutes.

Regardless of the method used for probe generation, the probe was purified using a Qiagen PCR cleanup kit (Qiagen, Valencia, Calif., USA), and eluted with 100 ul EB (kit provided). The sample was loaded on a Microcon YM-30 (Millipore, Bedford, Mass., USA) spin column and concentrated to 4-5 ul in volume.

Probes for the maize microarrays were generated using the Fluorescent Linear Amplification Kit (cat. No. G2556A) from Agilent Technologies (Palo Alto, Calif.).

Hybridization and Wash Conditions

The following Hybridization and Washing Condition were developed: Hybridization Conditions:

Labeled probe was heated at 95° C. for 3 min and chilled on ice. Then 25 μl of the hybridization buffer which was warmed at 42 C was added to the probe, mixing by pipeting, to give a final concentration of:
50% formamide
   4×SSC
   0.03% SDS
5× Denhardt's solution
0.1 μg/ml single-stranded salmon sperm DNA

The probe was kept at 42 C. Prior to the hybridization, the probe was heated for 1 more min., added to the array, and then covered with a glass cover slip. Slides were placed in hybridization chambers (Telechem, Sunnyvale, Calif.) and incubated at 42° C. overnight.

Washing Conditions:

A. Slides were washed in 1×SSC+0.03% SDS solution at room temperature for 5 minutes,
B. Slides were washed in 0.2×SSC at room temperature for 5 minutes,
C. Slides were washed in 0.05×SSC at room temperature for 5 minutes.

After A, B, and C, slides were spun at 800×g for 2 min. to dry. They were then scanned.

Maize microarrays were hybridized according to the instructions included Fluorescent Linear Amplification Kit (cat. No. G2556A) from Agilent Technologies (Palo Alto, Calif.).

Scanning of Slides

The chips were scanned using a ScanArray 3000 or 5000 (General Scanning, Watertown, Mass., USA). The chips were scanned at 543 and 633 nm, at 10 um resolution to measure the intensity of the two fluorescent dyes incorporated into the samples hybridized to the chips.

Data Extraction and Analysis

The images generated by scanning slides consisted of two 16-bit TIFF images representing the fluorescent emissions of the two samples at each arrayed spot. These images were then quantified and processed for expression analysis using the data extraction software Imagene™ (Biodiscovery, Los Angeles, Calif., USA). Imagene output was subsequently analyzed using the analysis program Genespring™ (Silicon Genetics, San Carlos, Calif., USA). In Genespring, the data was imported using median pixel intensity measurements derived from Imagene output. Background subtraction, ratio calculation and normalization were all conducted in Genespring. Normalization was achieved by breaking the data in to 32 groups, each of which represented one of the 32 pin printing regions on the microarray. Groups consist of 360 to 550 spots. Each group was independently normalized by setting the median of ratios to one and multiplying ratios by the appropriate factor.

Results

TABLE 3 presents the results of the differential expression experiments for the mRNAs, as reported by their corresponding cDNA ID number, that were differentially transcribed under a particular set of conditions as compared to a control sample. The cDNA ID numbers correspond to those utilized in the Reference and Sequence Tables. Increases in mRNA abundance levels in experimental plants versus the controls are denoted with the plus sign (+). Likewise, reductions in mRNA abundance levels in the experimental plants are denoted with the minus (−) sign.

The Table is organized according to the clone number with each set of experimental conditions being denoted by the term “Expt Rep ID:” followed by a “short name”. TABLE 3 links each Expt Rep ID with a short description of the experiment and the parameters. The experiment numbers are referenced in the appropriate utility/functions sections herein.

The sequences showing differential expression in a particular experiment (denoted by either a “+” or “−” in the Table) thereby shows utility for a function in a plant, and these functions/utilities are described in detail below, where the title of each section (i.e. a “utlity section”) is correlated with the particular differential expression experiment in TABLE 3.

Organ-Affecting Genes, Gene Components, Products (Including Differentiation and Function)

Root Genes

The economic values of roots arise not only from harvested adventitious roots or tubers, but also from the ability of roots to funnel nutrients to support growth of all plants and increase their vegetative material, seeds, fruits, etc. Roots have four main functions. First, they anchor the plant in the soil. Second, they facilitate and regulate the molecular signals and molecular traffic between the plant, soil, and soil fauna. Third, the root provides a plant with nutrients gained from the soil or growth medium. Fourth, they condition local soil chemical and physical properties.

Root genes are active or potentially active to a greater extent in roots than in most other organs of the plant. These genes and gene products can regulate many plant traits from yield to stress tolerance. Root genes can be used to modulate root growth and development.

Differential Expression of the Sequences in Roots

The relative levels of mRNA product in the root versus the aerial portion of the plant was measured. Specifically, mRNA was isolated from roots and root tips of Arabidopsis plants and compared to mRNA isolated from the aerial portion of the plants utilizing microarray procedures. Results are presented in TABLE 3.

Root Hair Genes, Gene Components and Products

Root hairs are specialized outgrowths of single epidermal cells termed trichoblasts. In many and perhaps all species of plants, the trichoblasts are regularly arranged around the perimeter of the root. In Arabidopsis, for example, trichoblasts tend to alternate with non-hair cells or atrichoblasts. This spatial patterning of the root epidermis is under genetic control, and a variety of mutants have been isolated in which this spacing is altered or in which root hairs are completely absent.

The root hair development genes of the instant invention are useful to modulate one or more processes of root hair structure and/or function including (1) development; (2) interaction with the soil and soil contents; (3) uptake and transport in the plant; and (4) interaction with microorganisms.

1.) Development

The surface cells of roots can develop into single epidermal cells termed trichoblasts or root hairs. Some of the root hairs will persist for the life of the plant; others will gradually die back; some may cease to function due to external influences. These genes and gene products can be used to modulate root hair density or root hair growth; including rate, timing, direction, and size, for example. These genes and gene products can also be used to modulate cell properties such as cell size, cell division, rate and direction and number, cell elongation, cell differentiation, lignified cell walls, epidermal cells (including trichoblasts) and root apical meristem cells (growth and initiation); and root hair architecture such as leaf cells under the trichome, cells forming the base of the trichome, trichome cells, and root hair responses. In addition these genes and gene products can be used to modulate one or more of the growth and development processes in response to internal plant programs or environmental stimuli in, for example, the seminal system, nodal system, hormone responses, Auxin, root cap abscission, root senescence, gravitropism, coordination of root growth and development with that of other organs (including leaves, flowers, seeds, fruits, and stems), and changes in soil environment (including water, minerals, Ph, and microfauna and flora).

2.) Interaction with Soil and Soil Contents

Root hairs are sites of intense chemical and biological activity and as a result can strongly modify the soil they contact. Roots hairs can be coated with surfactants and mucilage to facilitate these activities. Specifically, roots hairs are responsible for nutrient uptake by mobilizing and assimilating water, reluctant ions, organic and inorganic compounds and chemicals. In addition, they attract and interact with beneficial microfauna and flora. Root hairs also help to mitigate the effects of toxic ions, pathogens and stress. Thus, root hair genes and gene products can be used to modulate traits such as root hair surfactant and mucilage (including composition and secretion rate and time); nutrient uptake (including water, nitrate and other sources of nitrogen, phosphate, potassium, and micronutrients (e.g. iron, copper, etc.); microbe and nematode associations (such as bacteria including nitrogen-fixing bacteria, mycorrhizae, nodule-forming and other nematodes, and nitrogen fixation); oxygen transpiration; detoxification effects of iron, aluminum, cadium, mercury, salt, and other soil constituents; pathogens (including chemical repellents) glucosinolates (GSL1), which release pathogen-controlling isothiocyanates; and changes in soil (such as Ph, mineral excess and depletion), and rhizosheath.

3.) Transport of Materials in Plants

Uptake of the nutrients by the root and root hairs contributes a source-sink effect in a plant. The greater source of nutrients, the more sinks, such as stems, leaves, flowers, seeds, fruits, etc. can draw sustenance to grow. Thus, root hair development genes and gene products can be used to modulate the vigor and yield of the overall plant as well as distinct cells, organs, or tissues of a plant. The genes and gene products, therefore, can modulate plant nutrition, growth rate (such as whole plant, including height, flowering time, etc., seedling, coleoptile elongation, young leaves, stems, flowers, seeds and fruit) and yield, including biomass (fresh and dry weight during any time in plant life, including maturation and senescence), number of flowers, number of seeds, seed yield, number, size, weight and harvest index (content and composition, e.g. amino acid, jasmonate, oil, protein and starch) and fruit yield (number, size, weight, harvest index, and post harvest quality).

Reproduction Genes, Gene Components and Products

Reproduction genes are defined as genes or components of genes capable of modulating any aspect of sexual reproduction from flowering time and inflorescence development to fertilization and finally seed and fruit development. These genes are of great economic interest as well as biological importance. The fruit and vegetable industry grosses over $1 billion USD a year. The seed market, valued at approximately $15 billion USD annually, is even more lucrative.

Inflorescence and Floral Development Genes Gene Components and Products

During reproductive growth the plant enters a program of floral development that culminates in fertilization, followed by the production of seeds. Senescence may or may not follow. The flower formation is a precondition for the sexual propagation of plants and is therefore essential for the propagation of plants that cannot be propagated vegetatively as well as for the formation of seeds and fruits. The point of time at which the merely vegetative growth of plants changes into flower formation is of vital importance for example in agriculture, horticulture and plant breeding. Also the number of flowers is often of economic importance, for example in the case of various useful plants (tomato, cucumber, zucchini, cotton etc.) with which an increased number of flowers may lead to an increased yield, or in the case of growing ornamental plants and cut flowers.

Flowering plants exhibit one of two types of inflorescence architecture: indeterminate, in which the inflorescence grows indefinitely, or determinate, in which a terminal flower is produced. Adult organs of flowering plants develop from groups of stem cells called meristems. The identity of a meristem is inferred from structures it produces: vegetative meristems give rise to roots and leaves, inflorescence meristems give rise to flower meristems, and flower meristems give rise to floral organs such as sepals and petals. Not only are meristems capable of generating new meristems of different identity, but their own identity can change during development. For example, a vegetative shoot meristem can be transformed into an inflorescence meristem upon floral induction, and in some species, the inflorescence meristem itself will eventually become a flower meristem. Despite the importance of meristem transitions in plant development, little is known about the underlying mechanisms.

Following germination, the shoot meristem produces a series of leaf meristems on its flanks. However, once floral induction has occurred, the shoot meristem switches to the production of flower meristems. Flower meristems produce floral organ primordia, which develop individually into sepals, petals, stamens or carpels. Thus, flower formation can be thought of as a series of distinct developmental steps, i.e. floral induction, the formation of flower primordia and the production of flower organs. Mutations disrupting each of the steps have been isolated in a variety of species, suggesting that a genetic hierarchy directs the flowering process (see for review, Weigel and Meyerowitz, In Molecular Basis of Morphogenesis (ed. M. Bernfield). 51st Annual Symposium of the Society for Developmental Biology, pp. 93-107, New York, 1993).

Expression of many reproduction genes and gene products is orchestrated by internal programs or the surrounding environment of a plant. These genes can be used to modulate traits such as fruit and seed yield

Seed and Fruit Development Genes, Gene Components and Products

The ovule is the primary female sexual reproductive organ of flowering plants. At maturity it contains the egg cell and one large central cell containing two polar nuclei encased by two integuments that, after fertilization, develops into the embryo, endosperm, and seed coat of the mature seed, respectively. As the ovule develops into the seed, the ovary matures into the fruit or silique. As such, seed and fruit development requires the orchestrated transcription of numerous polynucleotides, some of which are ubiquitous, others that are embryo-specific and still others that are expressed only in the endosperm, seed coat, or fruit. Such genes are termed fruit development responsive genes and can be used to modulate seed and fruit growth and development such as seed size, seed yield, seed composition and seed dormancy.

Differential Expression of the Sequences in Siliques, Inflorescences and Flowers

The relative levels of mRNA product in the siliques relative to the plant as a whole was measured. The results are presented in TABLE 2.

Differential Expression of the Sequences in Hybrid Seed Development

The levels of mRNA product in the seeds relative to those in a leaf and floral stems was measured. The results are presented TABLE 2.

Development Genes, Gene Components and Products

Imbibition and Germination Responsive Genes, Gene Components and Products

Seeds are a vital component of the world's diet. Cereal grains alone, which comprise ˜90% of all cultivated seeds, contribute up to half of the global per capita energy intake. The primary organ system for seed production in flowering plants is the ovule. At maturity, the ovule consists of a haploid female gametophyte or embryo sac surrounded by several layers of maternal tissue including the nucleus and the integuments. The embryo sac typically contains seven cells including the egg cell, two synergids, a large central cell containing two polar nuclei, and three antipodal cells. That pollination results in the fertilization of both egg and central cell. The fertilized egg develops into the embryo. The fertilized central cell develops into the endosperm. And the integuments mature into the seed coat. As the ovule develops into the seed, the ovary matures into the fruit or silique. Late in development, the developing seed ends a period of extensive biosynthetic and cellular activity and begins to desiccate to complete its development and enter a dormant, metabolically quiescent state. Seed dormancy is generally an undesirable characteristic in agricultural crops, where rapid germination and growth are required. However, some degree of dormancy is advantageous, at least during seed development. This is particularly true for cereal crops because it prevents germination of grains while still on the ear of the parent plant (preharvest sprouting), a phenomenon that results in major losses to the agricultural industry. Extensive domestication and breeding of crop species have ostensibly reduced the level of dormancy mechanisms present in the seeds of their wild ancestors, although under some adverse environmental conditions, dormancy may reappear. By contrast, weed seeds frequently mature with inherent dormancy mechanisms that allow some seeds to persist in the soil for many years before completing germination.

Germination commences with imbibition, the uptake of water by the dry seed, and the activation of the quiescent embryo and endosperm. The result is a burst of intense metabolic activity. At the cellular level, the genome is transformed from an inactive state to one of intense transcriptional activity. Stored lipids, carbohydrates and proteins are catabolized fueling seedling growth and development. DNA and organelles are repaired, replicated and begin functioning. Cell expansion and cell division are triggered. The shoot and root apical meristem are activated and begin growth and organogenesis. Schematic 4 summarizes some of the metabolic and cellular processes that occur during imbibition. Germination is complete when a part of the embryo, the radicle, extends to penetrate the structures that surround it. In Arabidopsis, seed germination takes place within twenty-four (24) hours after imbibition. As such, germination requires the rapid and orchestrated transcription of numerous polynucleotides. Germination is followed by expansion of the hypocotyl and opening of the cotyledons. Meristem development continues to promote root growth and shoot growth, which is followed by early leaf formation.

Imbibition And Germination Genes

Imbibition and germination includes those events that commence with the uptake of water by the quiescent dry seed and terminate with the expansion and elongation of the shoots and roots. The germination period exists from imbibition to when part of the embryo, usually the radicle, extends to penetrate the seed coat that surrounds it. Imbibition and germination genes are defined as genes, gene components and products capable of modulating one or more processes of imbibition and germination described above. They are useful to modulate many plant traits from early vigor to yield to stress tolerance.

Differential Expression of the Sequences in Germinating Seeds and Imbibed Embryos

The levels of mRNA product in the seeds versus the plant as a whole was measured. The results are presented in TABLE 2.

Hormone Responsive Genes, Gene Components and Products

Abscissic Acid Responsive Genes, Gene Components and Products

Plant hormones are naturally occurring substances, effective in very small amounts, which act as signals to stimulate or inhibit growth or regulate developmental processes in plants. Abscisic acid (ABA) is a ubiquitous hormone in vascular plants that has been detected in every major organ or living tissue from the root to the apical bud. The major physiological responses affected by ABA are dormancy, stress stomatal closure, water uptake, abscission and senescence. In contrast to Auxins, cytokinins and gibberellins, which are principally growth promoters, ABA primarily acts as an inhibitor of growth and metabolic processes.

Changes in ABA concentration internally or in the surrounding environment in contact with a plant results in modulation of many genes and gene products. These genes and/or products are responsible for effects on traits such as plant vigor and seed yield.

While ABA responsive polynucleotides and gene products can act alone, combinations of these polynucleotides also affect growth and development. Useful combinations include different ABA responsive polynucleotides and/or gene products that have similar transcription profiles or similar biological activities, and members of the same or similar biochemical pathways. Whole pathways or segments of pathways are controlled by transcription factor proteins and proteins controlling the activity of signal transduction pathways. Therefore, manipulation of such protein levels is especially useful for altering phenotypes and biochemical activities of plants. In addition, the combination of an ABA responsive polynucleotide and/or gene product with another environmentally responsive polynucleotide is also useful because of the interactions that exist between hormone-regulated pathways, stress and defence induced pathways, nutritional pathways and development.

Differential Expression of the Sequences in ABA Treated Plants

The relative levels of mRNA product in plants treated with ABA versus controls treated with water were measured. Results are presented in TABLE 2.

Brassinosteroid Responsive Genes, Gene Components and Products

Plant hormones are naturally occuring substances, effective in very small amounts, which act as signals to stimulate or inhibit growth or regulate developmental processes in plants. Brassinosteroids (BRs) are the most recently discovered, and least studied, class of plant hormones. The major physiological response affected by BRs is the longitudinal growth of young tissue via cell elongation and possibly cell division. Consequently, disruptions in BR metabolism, perception and activity frequently result in a dwarf phenotype. In addition, because BRs are derived from the sterol metabolic pathway, any perturbations to the sterol pathway can affect the BR pathway. In the same way, perturbations in the BR pathway can have effects on the later part of the sterol pathway and thus the sterol composition of membranes.

Changes in BR concentration in the surrounding environment or in contact with a plant result in modulation of many genes and gene products. These genes and/or products are responsible for effects on traits such as plant biomass and seed yield. These genes were discovered and characterized from a much larger set of genes by experiments designed to find genes whose mRNA abundance changed in response to application of BRs to plants.

While BR responsive polynucleotides and gene products can act alone, combinations of these polynucleotides also affect growth and development. Useful combinations include different BR responsive polynucleotides and/or gene products that have similar transcription profiles or similar biological activities, and members of the same or functionally related biochemical pathways. Whole pathways or segments of pathways are controlled by transcription factors and proteins controlling the activity of signal transduction pathways. Therefore, manipulation of such protein levels is especially useful for altering phenotypes and biochemical activities of plants. In addition, the combination of a BR responsive polynucleotide and/or gene product with another environmentally responsive polynucleotide is useful because of the interactions that exist between hormone-regulated pathways, stress pathways, nutritional pathways and development. Here, in addition to polynucleotides having similar transcription profiles and/or biological activities, useful combinations include polynucleotides that may have different transcription profiles but which participate in common or overlapping pathways.

Differential Expression of the Sequences in Epi-Brassinolide or Brassinozole Plants

The relative levels of mRNA product in plants treated with either epi-brassinolide or brassinozole were measured. Results are presented in TABLE 2.

Metabolism Affecting Genes, Gene Components and Products

Nitrogen Responsive Genes, Gene Components and Products

Nitrogen is often the rate-limiting element in plant growth, and all field crops have a fundamental dependence on exogenous nitrogen sources. Nitrogenous fertilizer, which is usually supplied as ammonium nitrate, potassium nitrate, or urea, typically accounts for 40% of the costs associated with crops, such as corn and wheat in intensive agriculture. Increased efficiency of nitrogen use by plants should enable the production of higher yields with existing fertilizer inputs and/or enable existing yields of crops to be obtained with lower fertilizer input, or better yields on soils of poorer quality. Also, higher amounts of proteins in the crops could also be produced more cost-effectively. “Nitrogen responsive” genes and gene products can be used to alter or modulate plant growth and development.

Differential Expression of the Sequences in Whole Seedlings, Shoots and Roots

The relative levels of mRNA product in whole seedlings, shoots and roots treated with either high or low nitrogen media were compared to controls. Results are presented in TABLE 2.

Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. Viability genes can be modulated to affect cell or plant death. Herbicides are, by definition, chemicals that cause death of tissues, organs and whole plants. The genes and pathways that are activated or inactivated by herbicides include those that cause cell death as well as those that function to provide protection.

Differential Expression of the Sequences in Herbicide Treated Plants and Herbicide Resistant Mutants

The relative levels of mRNA product in plants treated with heribicide and mutants resistant to heribicides were compared to control plants. Results are presented in TABLE 2.

Stress Responsive Genes, Gene Components and Products

Wounding Responsive Genes, Gene Components and Products

Plants are continuously subjected to various forms of wounding from physical attacks including the damage created by pathogens and pests, wind, and contact with other objects. Therefore, survival and agricultural yields depend on constraining the damage created by the wounding process and inducing defense mechanisms against future damage.

Plants have evolved complex systems to minimize and/or repair local damage and to minimize subsequent attacks by pathogens or pests or their effects. These involve stimulation of cell division and cell elongation to repair tissues, induction of programmed cell death to isolate the damage caused mechanically and by invading pests and pathogens, and induction of long-range signaling systems to induce protecting molecules, in case of future attack. The genetic and biochemical systems associated with responses to wounding are connected with those associated with other stresses such as pathogen attack and drought.

Wounding responsive genes and gene products can be used to alter or modulate traits such as growth rate; whole plant height, width, or flowering time; organ development (such as coleoptile elongation, young leaves, roots, lateral roots, tuber formation, flowers, fruit, and seeds); biomass; fresh and dry weight during any time in plant life, such as at maturation; number of flowers; number of seeds; seed yield, number, size, weight, harvest index (such as content and composition, e.g., amino acid, nitrogen, oil, protein, and carbohydrate); fruit yield, number, size, weight, harvest index, post harvest quality, content and composition (e.g., amino acid, carotenoid, jasmonate, protein, and starch); seed and fruit development; germination of dormant and non-dormant seeds; seed viability, seed reserve mobilization, fruit ripening, initiation of the reproductive cycle from a vegetative state, flower development time, insect attraction for fertilization, time to fruit maturity, senescence; fruits, fruit drop; leaves; stress and disease responses; drought; heat and cold; wounding by any source, including wind, objects, pests and pathogens; uv and high light damage (insect, fungus, virus, worm, nematode damage).

Cold Responsive Genes, Gene Components and Products

The ability to endure low temperatures and freezing is a major determinant of the geographical distribution and productivity of agricultural crops. Even in areas considered suitable for the cultivation of a given species or cultivar, can give rise to yield decreases and crop failures as a result of aberrant, freezing temperatures. Even modest increases (1-2° C.) in the freezing tolerance of certain crop species would have a dramatic impact on agricultural productivity in some areas. The development of genotypes with increased freezing tolerance would provide a more reliable means to minimize crop losses and diminish the use of energy-costly practices to modify the microclimate.

Sudden cold temperatures result in modulation of many genes and gene products, including promoters. These genes and/or products are responsible for effects on traits such as plant vigor and seed yield.

Manipulation of one or more cold responsive gene activities is useful to modulate growth and development.

Differential Expression of the Sequences in Cold Treated Plants

The relative levels of mRNA product in cold treated plants were compared to control plants. Results are presented in TABLE 2.

Heat Responsive Genes, Gene Components and Products

The ability to endure high temperatures is a major determinant of the geographical distribution and productivity of agricultural crops. Decreases in yield and crop failure frequently occur as a result of aberrant, hot conditions even in areas considered suitable for the cultivation of a given species or cultivar. Only modest increases in the heat tolerance of crop species would have a dramatic impact on agricultural productivity. The development of genotypes with increased heat tolerance would provide a more reliable means to minimize crop losses and diminish the use of energy-costly practices to modify the microclimate.

Changes in temperature in the surrounding environment or in a plant microclimate results in modulation of many genes and gene products.

Differential Expression of the Sequences in Heat Treated Plants

The relative levels of mRNA product in heat treated plants were compared to control plants. Results are presented in TABLE 2.

Drought Responsive Genes, Gene Components and Products

The ability to endure drought conditions is a major determinant of the geographical distribution and productivity of agricultural crops. Decreases in yield and crop failure frequently occur as a result of aberrant, drought conditions even in areas considered suitable for the cultivation of a given species or cultivar. Only modest increases in the drought tolerance of crop species would have a dramatic impact on agricultural productivity. The development of genotypes with increased drought tolerance would provide a more reliable means to minimize crop losses and diminish the use of energy-costly practices to modify the microclimate.

Drought conditions in the surrounding environment or within a plant, results in modulation of many genes and gene products.

Differential Expression of the Sequences in Drought Treated Plants and Drought Mutants

The relative levels of mRNA product in drought treated plants and drought mutants were compared to control plants. Results are presented in TABLE 2.

Methyl Jasmonate (Jasmonate) Responsive Genes, Gene Components and Products

Jasmonic acid and its derivatives, collectively referred to as jasmonates, are naturally occurring derivatives of plant lipids. These substances are synthesized from linolenic acid in a lipoxygenase-dependent biosynthetic pathway. Jasmonates are signalling molecules which have been shown to be growth regulators as well as regulators of defense and stress responses. As such, jasmonates represent a separate class of plant hormones. Jasmonate responsive genes can be used to modulate plant growth and development.

Differential Expression of the Sequences in Methyl Jasmonate Treated Plants

The relative levels of mRNA product in methyl jasmonate treated plants were compared to control plants. Results are presented in TABLE 2.

Salicylic Acid Responsive Genes, Gene Components and Products

Plant defense responses can be divided into two groups: constitutive and induced. Salicylic acid (SA) is a signaling molecule necessary for activation of the plant induced defense system known as systemic acquired resistance or SAR. This response, which is triggered by prior exposure to avirulent pathogens, is long lasting and provides protection against a broad spectrum of pathogens. Another induced defense system is the hypersensitive response (HR). HR is far more rapid, occurs at the sites of pathogen (avirulent pathogens) entry and precedes SAR. SA is also the key signaling molecule for this defense pathway.

Differential Expression of the Sequences in Salicylic Acid Treated Plants

The relative levels of mRNA product in salicylic acid treated plants were compared to control plants. Results are presented in TABLE 2.

Osmotic Stress Responsive Genes, Gene Components and Products

The ability to endure and recover from osmotic and salt related stress is a major determinant of the geographical distribution and productivity of agricultural crops. Osmotic stress is a major component of stress imposed by saline soil and water deficit. Decreases in yield and crop failure frequently occur as a result of aberrant or transient environmental stress conditions even in areas considered suitable for the cultivation of a given species or cultivar. Only modest increases in the osmotic and salt tolerance of a crop species would have a dramatic impact on agricultural productivity. The development of genotypes with increased osmotic tolerance would provide a more reliable means to minimize crop losses and diminish the use of energy-costly practices to modify the soil environment. Thus, osmotic stress responsive genes can be used to modulate plant growth and development.

Differential Expression of the Sequences in PEG Treated Plants

The relative levels of mRNA product in PEG treated plants were compared to control plants. Results are presented in TABLE 2.

Shade Responsive Genes, Gene Components and Products

Plants sense the ratio of Red (R):Far Red (FR) light in their environment and respond differently to particular ratios. A low R:FR ratio, for example, enhances cell elongation and favors flowering over leaf production. The changes in R:FR ratios mimic and cause the shading response effects in plants. The response of a plant to shade in the canopy structures of agricultural crop fields influences crop yields significantly. Therefore manipulation of genes regulating the shade avoidance responses can improve crop yields. While phytochromes mediate the shade avoidance response, the down-stream factors participating in this pathway are largely unknown. One potential downstream participant, ATHB-2, is a member of the HD-Zip class of transcription factors and shows a strong and rapid response to changes in the R:FR ratio. ATHB-2 overexpressors have a thinner root mass, smaller and fewer leaves and longer hypocotyls and petioles. This elongation arises from longer epidermal and cortical cells, and a decrease in secondary vascular tissues, paralleling the changes observed in wild-type seedlings grown under conditions simulating canopy shade. On the other hand, plants with reduced ATHB-2 expression have a thick root mass and many larger leaves and shorter hypocotyls and petioles. Here, the changes in the hypocotyl result from shorter epidermal and cortical cells and increased proliferation of vascular tissue. Interestingly, application of Auxin is able to reverse the root phenotypic consequences of high ATHB-2 levels, restoring the wild-type phenotype. Consequently, given that ATHB-2 is tightly regulated by phytochrome, these data suggest that ATHB-2 may link the Auxin and phytochrome pathways in the shade avoidance response pathway.

Shade responsive genes can be used to modulate plant growth and development.

Differential Expression of the Sequences in Far-Red Light Treated Plants

The relative levels of mRNA product in far-red light treated plants were compared to control plants. Results are presented in TABLE 2.

Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. The applicants have elucidated many such genes and pathways by discovering genes that when inactivated lead to cell or plant death.

Herbicides are, by definition, chemicals that cause death of tissues, organs and whole plants. The genes and pathways that are activated or inactivated by herbicides include those that cause cell death as well as those that function to provide protection. The applicants have elucidated these genes.

The genes defined in this section have many uses including manipulating which cells, tissues and organs are selectively killed, which are protected, making plants resistant to herbicides, discovering new herbicides and making plants resistant to various stresses.

Viability genes were also identified from a much larger set of genes by experiments designed to find genes whose mRNA products changed in concentration in response to applications of different herbicides to plants. Viability genes are characteristically differentially transcribed in response to fluctuating herbicide levels or concentrations, whether internal or external to an organism or cell. The MA_diff Table reports the changes in tanscript levels of various viability genes.

Early Seedling-Phase Specific Responsive Genes, Gene Components and Products

One of the more active stages of the plant life cycle is a few days after germination is complete, also referred to as the early seedling phase. During this period the plant begins development and growth of the first leaves, roots, and other organs not found in the embryo. Generally this stage begins when germination ends. The first sign that germination has been completed is usually that there is an increase in length and fresh weight of the radicle. Such genes and gene products can regulate a number of plant traits to modulate yield. For example, these genes are active or potentially active to a greater extent in developing and rapidly growing cells, tissues and organs, as exemplified by development and growth of a seedling 3 or 4 days after planting a seed.

Rapid, efficient establishment of a seedling is very important in commercial agriculture and horticulture. It is also vital that resources are approximately partitioned between shoot and root to facilitate adaptive growth. Phototropism and geotropism need to be established. All these require post-germination process to be sustained to ensure that vigorous seedlings are produced. Early seedling phase genes, gene components and products are useful to manipulate these and other processes.

Scattered throughout the epidermis of the shoot are minute pores called stomata. Each stomal pore is surrounded by two guard cells. The guard cells control the size of the stomal pore, which is critical since the stomata control the exchange of carbon dioxide, oxygen, and water vapor between the interior of the plant and the outside atmosphere. Stomata open and close through turgor changes driven by ion fluxes, which occur mainly through the guard cell plasma membrane and tonoplast. Guard cells are known to respond to a number of external stimuli such as changes in light intensity, carbon dioxide and water vapor, for example. Guard cells can also sense and rapidly respond to internal stimuli including changes in ABA, auxin and calcium ion flux.

Thus, genes, gene products, and fragments thereof differentially transcribed and/or translated in guard cells can be useful to modulate ABA responses, drought tolerance, respiration, water potential, and water management as examples. All of which can in turn affect plant yield including seed yield, harvest index, fruit yield, etc.

To identify such guard cell genes, gene products, and fragments thereof, Applicants have performed a microarray experiment comparing the transcript levels of genes in guard cells versus leaves. Experimental data is shown below.

Nitric Oxide Responsive Genes, Gene Components and Products

The rate-limiting element in plant growth and yield is often its ability to tolerate suboptimal or stress conditions, including pathogen attack conditions, wounding and the presence of various other factors. To combat such conditions, plant cells deploy a battery of inducible defense responses, including synergistic interactions between nitric oxide (NO), reactive oxygen intermediates (ROS), and salicylic acid (SA). NO has been shown to play a critical role in the activation of innate immune and inflammatory responses in animals. At least part of this mammalian signaling pathway is present in plants, where NO is known to potentiate the hypersensitive response (HR). In addition, NO is a stimulator molecule in plant photomorphogenesis.

Changes in nitric oxide concentration in the internal or surrounding environment, or in contact with a plant, results in modulation of many genes and gene products.

In addition, the combination of a nitric oxide responsive polynucleotide and/or gene product with other environmentally responsive polynucleotides is also useful because of the interactions that exist between hormone regulated pathways, stress pathways, pathogen stimulated pathways, nutritional pathways and development.

Nitric oxide responsive genes and gene products can function either to increase or dampen the above phenotypes or activities either in response to changes in nitric oxide concentration or in the absence of nitric oxide fluctuations. More specifically, these genes and gene products can modulate stress responses in an organism. In plants, these genes and gene products are useful for modulating yield under stress conditions. Measurments of yield include seed yield, seed size, fruit yield, fruit size, etc.

Shoot-Apical Meristem Genes, Gene Components and Products

New organs, stems, leaves, branches and inflorescences develop from the stem apical meristem (SAM). The growth structure and architecture of the plant therefore depends on the behavior of SAMs. Shoot apical meristems (SAMs) are comprised of a number of morphologically undifferentiated, dividing cells located at the tips of shoots. SAM genes elucidated here are capable of modifying the activity of SAMs and thereby many traits of economic interest from ornamental leaf shape to organ number to responses to plant density.

In addition, a key attribute of the SAM is its capacity for self-renewal. Thus, SAM genes of the instant invention are useful for modulating one or more processes of SAM structure and/or function including (I) cell size and division; (II) cell differentiation and organ primordia. The genes and gene components of this invention are useful for modulating any one or all of these cell division processes generally, as in timing and rate, for example. In addition, the polynucleotides and polypeptides of the invention can control the response of these processes to the internal plant programs associated with embryogenesis, and hormone responses, for example.

Because SAMs determine the architecture of the plant, modified plants will be useful in many agricultural, horticultural, forestry and other industrial sectors. Plants with a different shape, numbers of flowers and seed and fruits will have altered yields of plant parts. For example, plants with more branches can produce more flowers, seed or fruits. Trees without lateral branches will produce long lengths of clean timber. Plants with greater yields of specific plant parts will be useful sources of constituent chemicals.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

### EXAMPLE 2

### GFP Experimental Procedures and Results

Procedures

The polynucleotide sequences of the present invention were tested for promoter activity using Green Fluorescent Protein (GFP) assays in the following manner.

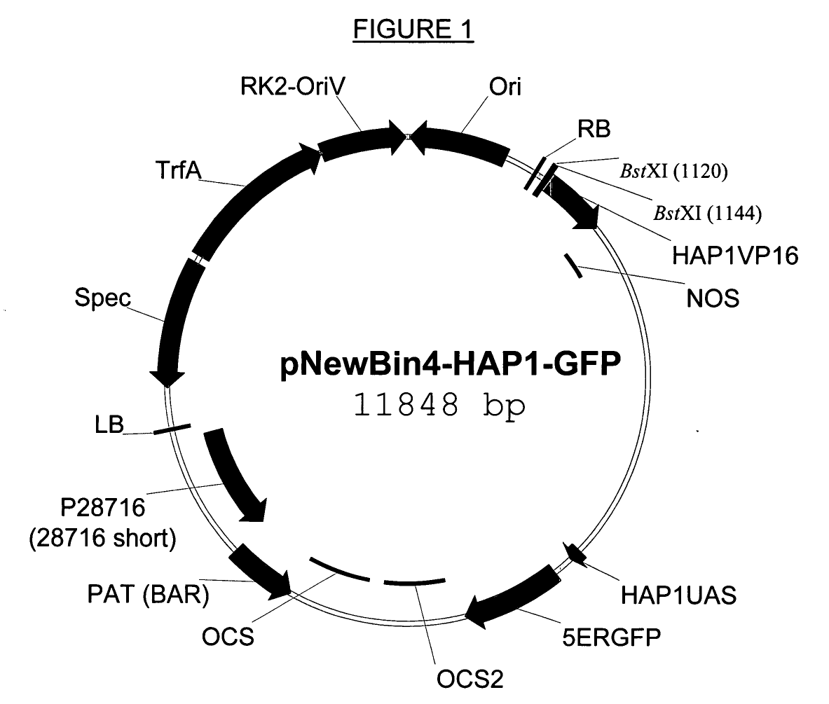

Approximately 1-2 kb of genomic sequence occurring immediately upstream of the ATG translational start site of the gene of interest was isolated using appropriate primers tailed with BstXI restriction sites. Standard PCR reactions using these primers and genomic DNA were conducted. The resulting product was isolated, cleaved with BstXI and cloned into the BstXI site of an appropriate vector, such as pNewBin4-HAP1-GFP (see FIG. 1).

Transformation

The following procedure was used for transformation of plants
1. Stratification of WS-2 Seed.
   Add 0.5 ml WS-2 (CS2360) seed to 50 ml of 0.2% Phytagar in a 50 ml Corning tube and vortex until seeds and Phytagar form a homogenous mixture.
   Cover tube with foil and stratify at 4° C. for 3 days.
2. Preparation of Seed Mixture.
   Obtain stratified seed from cooler.
   Add seed mixture to a 1000 ml beaker.
   Add an additional 950 ml of 0.2% Phytagar and mix to homogenize.
3. Preparation of Soil Mixture.
   Mix 24 L SunshineMix #5 soil with 16 L Therm-O-Rock vermiculite in cement mixer to make a 60:40 soil mixture.
   Amend soil mixture by adding 2 Tbsp Marathon and 3 Tbsp Osmocote and mix contents thoroughly.
   Add 1 Tbsp Peters fertilizer to 3 gallons of water and add to soil mixture and mix thoroughly.
   Fill 4-inch pots with soil mixture and round the surface to create a slight dome.
   Cover pots with 8-inch squares of nylon netting and fasten using rubber bands.
   Place 14 4-inch pots into each no-hole utility flat.
4. Planting.
   Using a 60 ml syringe, aspirate 35 ml of the seed mixture.
   Exude 25 drops of the seed mixture onto each pot.
   Repeat until all pots have been seeded.
   Place flats on greenhouse bench, cover flat with clear propagation domes, place 55% shade cloth on top of flats and subirrigate by adding 1 inch of water to bottom of each flat.
5. Plant Maintenance.
   3 to 4 days after planting, remove clear lids and shade cloth.
   Subirrigate flats with water as needed.
   After 7-10 days, thin pots to 20 plants per pot using forceps.
   After 2 weeks, subirrigate all plants with Peters fertilizer at a rate of 1 Tsp per gallon water.
   When bolts are about 5-10 cm long, clip them between the first node and the base of stem to induce secondary bolts.
   6 to 7 days after clipping, perform dipping infiltration.
6. Preparation of Agrobacterium.
   Add 150 ml fresh YEB to 250 ml centrifuge bottles and cap each with a foam plug (Identi-Plug).
   Autoclave for 40 min at 121° C.
   After cooling to room temperature, uncap and add 0.1 ml each of carbenicillin, spectinomycin and rifampicin stock solutions to each culture vessel.
   Obtain Agrobacterium starter block (96-well block with Agrobacterium cultures grown to an OD₆₀₀ of approximately 1.0) and inoculate one culture vessel per construct by transferring 1 ml from appropriate well in the starter block.
   Cap culture vessels and place on Lab-Line incubator shaker set at 27° C. and 250 RPM.
   Remove after Agrobacterium cultures reach an OD₆₀₀ of approximately 1.0 (about 24 hours), cap culture vessels with plastic caps, place in Sorvall SLA 1500 rotor and centrifuge at 8000 RPM for 8 min at 4° C.
   Pour out supernatant and put bottles on ice until ready to use.
   Add 200 ml Infiltration Media (IM) to each bottle, resuspend Agrobacterium pellets and store on ice.
7. Dipping Infiltration.
   Pour resuspended Agrobacterium into 16 oz polypropylene containers.
   Invert 4-inch pots and submerge the aerial portion of the plants into the Agrobacterium suspension and let stand for 5 min.
   Pour out Agrobacterium suspension into waste bucket while keeping polypropylene container in place and return the plants to the upright position.
   Place 10 covered pots per flat.
   Fill each flat with 1-inch of water and cover with shade cloth.
   Keep covered for 24 hr and then remove shade cloth and polypropylene containers.
   Resume normal plant maintenance.
   When plants have finished flowering cover each pot with a ciber plant sleeve.
   After plants are completely dry, collect seed and place into 2.0 ml micro tubes and store in 100-place cryogenic boxes.
      Recipes:
      0.2% Phytagar
   2 g Phytagar
   1 L nanopure water
      Shake until Phytagar suspended
      Autoclave 20 min
         YEB (for 1 L)
   5 g extract of meat
   5 g Bacto peptone
   1 g yeast extract
   5 g sucrose
   0.24 g magnesium sulfate
      While stirring, add ingredients, in order, to 900 ml nanopure water
      When dissolved, adjust pH to 7.2
      Fill to 1 L with nanopure water
      Autoclave 35 min
         Infiltration Medium (IM) (for 1 L)
   2.2 g MS salts
   50 g sucrose
   5 ul BAP solution (stock is 2 mg/ml)
      While stirring, add ingredients in order listed to 900 ml nanopure water
      When dissolved, adjust pH to 5.8.
      Volume up to 1 L with nanopure water.
      Add 0.02% Silwet L-77 just prior to resuspending Agrobacterium

High Throughput Screening—T1 Generation
1. Soil Preparation. Wear gloves at all times.
   In a large container, mix 60% autoclaved SunshineMix #5 with 40% vermiculite.
   Add 2.5 Tbsp of Osmocote, and 2.5 Tbsp of 1% granular Marathon per 25 L of soil.
   Mix thoroughly.
2. Fill Com-Packs With Soil.
   Loosely fill D601 Com-Packs level to the rim with the prepared soil.
   Place filled pot into utility flat with holes, within a no-hole utility flat.
   Repeat as necessary for planting. One flat set should contain 6 pots.
      3. Saturate Soil.
   Evenly water all pots until the soil is saturated and water is collecting in the bottom of the flats.
   After the soil is completely saturated, dump out the excess water.
      4. Plant the Seed.
      5. Stratify the Seeds.
   After sowing the seed for all the flats, place them into a dark 4° C. cooler.
   Keep the flats in the cooler for 2 nights for WS seed. Other ecotypes may take longer. This cold treatment will help promote uniform germination of the seed.
6. Remove Flats From Cooler and Cover With Shade Cloth. (Shade cloth is only needed in the greenhouse)
   After the appropriate time, remove the flats from the cooler and place onto growth racks or benches.
   Cover the entire set of flats with 55% shade cloth. The cloth is necessary to cut down the light intensity during the delicate germination period.
   The cloth and domes should remain on the flats until the cotyledons have fully expanded. This usually takes about 4-5 days under standard greenhouse conditions.
7. Remove 55% Shade Cloth and Propagation Domes.
   After the cotyledons have fully expanded, remove both the 55% shade cloth and propagation domes.
8. Spray Plants With Finale Mixture. Wear gloves and protective clothing at all times.
   Prepare working Finale mixture by mixing 3 ml concentrated Finale in 48 oz of water in the Poly-TEK sprayer.
   Completely and evenly spray plants with a fine mist of the Finale mixture.
   Repeat Finale spraying every 3-4 days until only transformants remain. (Approximately 3 applications are necessary.)
   When satisfied that only transformants remain, discontinue Finale spraying.
9. Weed Out Excess Transformants.
Weed out excess transformants such that a maximum number of five plants per pot exist evenly spaced throughout the pot.

GFP Assay

Tissues are dissected by eye or under magnification using INOX 5 grade forceps and placed on a slide with water and coversliped. An attempt is made to record images of observed expression patterns at earliest and latest stages of development of tissues listed below. Specific tissues will be preceded with High (H), Medium (M), Low (L) designations.

| | |
|---|---|
| [symbol] Flower | [symbol] pedicel [symbol] receptacle [symbol] nectary [symbol] sepal [symbol] petal [symbol] filament [symbol] anther [symbol] pollen [symbol] carpel [symbol] style [symbol] papillae [symbol] vascular |
| | [symbol] epidermis [symbol] stomata [symbol] trichome |
| [symbol] Silique | [symbol] stigma [symbol] style [symbol] carpel [symbol] septum [symbol] placentae [symbol] transmitting tissue [symbol] vascular [symbol] epidermis [symbol] stomata |
| | [symbol] abscission zone [symbol] ovule |
| [symbol] Ovule | Pre-fertilization: [symbol] inner integument [symbol] outer integument [symbol] embryo sac [symbol] funiculus [symbol] chalaza [symbol] micropyle |
| | [symbol] gametophyte |
| | Post-fertilization: [symbol] zygote [symbol] inner integument [symbol] outer integument [symbol] seed coat [symbol] primordia [symbol] chalaza |
| | [symbol] micropyle [symbol] early endosperm [symbol] mature endosperm [symbol] embryo |
| [symbol] Embryo | [symbol] suspensor [symbol] preglobular [symbol] globular [symbol] heart [symbol] torpedo [symbol] late [symbol] mature [symbol] provascular [symbol] hypophysis [symbol] radicle |
| | [symbol] cotyledons [symbol] hypocotyl |
| [symbol] Stem | [symbol] epidermis [symbol] cortex [symbol] vascular [symbol] xylem [symbol] phloem [symbol] pith [symbol] stomata [symbol] trichome |
| [symbol] Leaf | [symbol] petiole [symbol] mesophyll [symbol] vascular [symbol] epidermis [symbol] trichome [symbol] primordia [symbol] stomata [symbol] stipule [symbol] margin |

T1 Mature: These are the T1 plants resulting from independent transformation events. These are screened between stage 6.50-6.90 (means the plant is flowering and that 50-90% of the flowers that the plant will make have developed) which is 4-6 weeks of age. At this stage the mature plant possesses flowers, siliques at all stages of development, and fully expanded leaves. We do not generally differentiate between 6.50 and 6.90 in the report but rather just indicate 6.50. The plants are initially imaged under UV with a Leica Confocal microscope. This allows examination of the plants on a global level. If expression is present, they are imaged using scanning laser confocal micsrocopy.

T2 Seedling: Progeny are collected from the T1 plants giving the same expression pattern and the progeny (T2) are sterilized and plated on agar-solidified medium containing M&S salts. In the event that there was no expression in the T1 plants, T2 seeds are planted from all lines. The seedlings are grown in Percival incubators under continuous light at 22° C. for 10-12 days. Cotyledons, roots, hypocotyls, petioles, leaves, and the shoot meristem region of individual seedlings were screened until two seedlings were observed to have the same pattern. Generally found the same expression pattern was found in the first two seedlings. However, up to 6 seedlings were screened before “no expression pattern” was recorded. All constructs are screened as T2 seedlings even if they did not have an expression pattern in the T1 generation.

T2 Mature: The T2 mature plants were screened in a similar manner to the T1 plants. The T2 seeds were planted in the greenhouse, exposed to selection and at least one plant screened to confirm the T1 expression pattern. In instances where there were any subtle changes in expression, multiple plants were examined and the changes noted in the tables.

T3 Seedling: This was done similar to the T2 seedlings except that only the plants for which we are trying to confirm the pattern are planted.

Image Data:

Images are collected by scanning laser confocal microscopy. Scanned images are taken as 2-D optical sections or 3-D images generated by stacking the 2-D optical sections collected in series. All scanned images are saved as TIFF files by imaging software, edited in Adobe Photoshop, and labeled in Powerpoint specifying organ and specific expressing tissues.

Instrumentation:

Microscope

Inverted Leica DM IRB
Fluorescence filter blocks:
Blue excitation BP 450-490; long pass emission LP 515.
   A. Green excitation BP 515-560; long pass emission LP 590
      Objectives
HC PL FLUOTAR 5×/0.5
   B. HCPL APO 10×/0.4 IMM water/glycerol/oil
HCPL APO 20×/0.7 IMM water/glycerol/oil
HCXL APO 63×/1.2 IMM water/glycerol/oil
   Leica TCS SP2 Confocal Scanner
Spectral range of detector optics 400-850 nm.
Variable computer controlled pinhole diameter.
Optical zoom 1-32×.
Four simultaneous detectors:
Three channels for collection of fluorescence or reflected light.
One channel for transmitted light detector.
Laser sources:
Blue Ar 458/5 mW, 476 nm/5 mW, 488 nm/20 mW, 514 nm/20 mW.
Green HeNe 543 nm/1.2 mW
Red HeNe 633 nm/10 mW
   Results

Table 2 presents the results of the GFP assays as reported by the corresponding cDNA ID number, construct number and line number. Unlike the microarray results, which measure the difference in expression of the endogenous cDNA under various conditions, the GFP data gives the location of expression that is visible under the imaging parameters.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

TABLE 1

>4905097_construct_ID_YP0103
ATAGCAAACAATCACATCATCGCAATATACATAAACAAAAGAGGAAGAAAAATGGCAACCGAGTGGTGTAGTTATATTGG
GAAGAACTCATGGCCGGAGCTTTTAGGAACAAATGGAGACTATGCGGCTTCGGTGATAAAAGGAGAGAACTCGAGCCTCA
ACGTTGTCGTGGTTTCGGATGGAAATTATGTGACTGAAGACCTCAGTTGCTACCGCGTTAGGGTTTGGGTTGACGAAATC
CGTATCGTTGTCAGAAACCCAACCGCCGGCTAGACATGTATATGGACCACCATTATGCTATAGCCATGTAGGCGCCTTAC
TATGAATAAATGAAACTATATATAATGCATGCATAGTTGGTTGGTTGGTCATAATGTAACATCTATTGTTTGCTTGAATG
ATTCTGGTGTCCGATCATATAACGCATTTGAATG
>4905097_protein_ID_4905099
MATEWCSYIGKNSWPELLGTNGDYAASVIKGENSSLNVVVVSDGNYVTEDLSCYRVRVWVDEIRIVVRNPTAG*
>4906343_construct_ID_YP0098
ACAAATCATTTTTCTTAGGATTTGTTTAGTAAAATAAAAATATTTCTTGTACATTTCAATCATAAGTAGATATGGCTAAA
TTTAACTCTCAGATTACTACGCTATTCATTGTTGTAGCTTTGGTGTGTGCATTTGTTCCAACTTTCTCAGTCAAAGAAGC
TGAAGCAAATTTATTATGGAATACTTGTCTTGTTAAATTCACTCCTAAGTGTGCGTTAGATATAATTGCTGCTGTCTTCG
AAAATGGAACAATGTCTGATCCTTGTTGCAACGATCTTGTCAAAGAAGGAAAAGTGTGTCACGATACGCTTATTAAATAT
ATTGCAGATAAACCCATGTTAATTGCTCACGAAACAGAATACTTGAAGAAGAGTGATGACTTGTGGATACATTGTGTCTC
AATCTCCAAAAGTGCTTGAAATGTATATTGCGTGTACTATTTTCACCCAATAAATTGATTGTTTTCTGTTGTTATAGTTT
TCTTCACACAAGCCTTTATATTTTAACTTAACAACAATTTTAACCAAAGCGAATTTCTTTCTTAAAAAGTATAACTTTAA
TTTATGATTATCTATTTGAACTCGAAACAAAATTTCTTATAAAGAGTCGAATAATAATTCAAAATTTAACTATTAAGAGG
AGCTCTAACTAATATTGTTTAGTGAAATTTAATTTTTGTATTTTCTTTCTAATTAGAGTAATAAGTTATTC
>4906343_protein_ID_4906344
MAKFNSQITTLFIVVALVCAFVPTFSVKEAEANLLWNTCLVKFTPKCALDIIAAVFENGTMSDPCCNDLVKEGKVCHDTL
IKYIADKPMLIAHETEYLKKSDDLWKHCVSISKSA*
>4909291_construct_ID_YP0019
AATTGTCTTATCTTTCGACTTTTCTTCTTCTTCTTCTTAAGAGATTTTTCTCCAAGAAAGTTCGCTCCTTTTCTCTGTTC
TTAACAAAAAAGTCTCGGTTTTTTTCTCTTTGTTTTGGGTACTAGCGTGATGTCTTCTGAGAATGATTTCGTTGAGTTTT
CTTCTATGTTCGAGAGAATTATACAAGGAAGAGGTGATGGTCTCTCTCGATTTTTGCCGGTGATTGTAGCTTTAGCCGCC
AGAGAAGACGATGATGACCAAGGATCTACCGATCAAACAACGAGACGGGGAGATCCGTTGAGTCCAAGGTTCGTGATGAT
CGGATCGCGATCGGGACTCGACGATTTCTTTAGCGACGGTGGAAAACAAGGGAGGTCGCCGGCGTTGAAGTCAGAAGTGG
AGAATATGCCACGTGTCGTGATCGGAGAAGATAAGGAGAAATATGGTGGTTCTTGCGCGATTTGTTTGGATGAGTGGTCT
AAAGGTGACGTGGCGGCGGAGATGCCTTGTAAACATAAGTTTCACTCAAAGTGTGTGGAGGAGTGGTTAGGGAGGCACGC
CACGTGTCCTATGTGTAGGTATGAGATGCCTGTTGAAGAAGTTGAAGAAGAGAAGAAGATTGGGATTTGGATTGGTTTCT
CCATTAACGCCGGCGACAGAAGAAACTAAGAAGACGGAGGAAGAAGAAGTTAAAAGTGACTCGAACCCTCAAGATGCAAC
ATGGGGCTAGGTTTAGGTTTAGGTTTGCTAGAATGTTTTGTATAGTTTCGTTTTCGTTTACTGAAATCAATTTCGAATTC
AATAAAATTGGTTGC
>4909291_protein_ID_4909292
MSSENDFVEFSSMFERIIQGRGDGLSRFLPVIVALAAREDDDDQGSTDQTTRRGDPLSPRFVMIGSRSGLDDFFSDGGKQ
GRSPALKSEVENMPRVVIGEDKEKYGGSCAICLDEWSKGDVAAEMPCKHKFHSKCVEEWLGRHATCPMCRYEMPVEEVEE
EKKIGIWIGFSINAGDRRN*
>4909806_construct_ID_YP0050
GTCTTGGCATCCTCGTCCTCTTCAGCAAAACTCGTCTCTCTTGCACTCCAAAAAGCAACCATGTCTGCTTTTGTCGGCAA
ATACGCAGATGAGCTGATAAAGACGGCTAAGTACATTGCCACACCGGGAAAGGGCATTTTGGCAGCAGACGAGAGCACGG
GAACTATTGGGAAACGATTCGCCAGCATCAATGTTGAGAACATTGAGTCCAACCGCCAAGCTCTCCGTGAGCTCCTCTTC
ACGTCCCCTGGCACTTTCCCTTGCCTCTCCGGTGTTATCCTCTTCGAGGAAACCCTCTACCAGAAAACCACGGATGGCAA
ACCCTTCGTTGAGCTCCTCATGGAAAACGGAGTTATCCCTGGAATCAAAGTGGACAAGGGTGTGGTTGATCTAGCAGGAA
CCAATGGCGAGACCACTACTCAGGGTCTAGATTCACTTGGTGCACGTTGCCAGGAGTATTACAAGGCAGGAGCTCGGTTT
GCAAAATGGCGTGCAGTCCTCAAGATTGGGGCCACCGAGCCAAGCGAGCTCTCTATCCAAGAGAACGCCAAGGGGCTAGC
CCGCTATGCCATCATCTGCCAGGAGAATGGACTCGTCCCAATCGTCGAGCCAGAGGTACTGACCGACGGGAGCCATGACA
TCAAGAAATGTGCAGCGGTGACCGAGACCGTTCTTGCTGCCGTGTACAAGGCCTTGAACGACCACCATGTCCTCCTCGAA
GGCACTCTGCTTAAACCGAACATGGTCACTCCCGGCTCTGACAGCCCAAAGGTTGCACCGGAAGTGATAGCGGAATACAC
AGTGACTGCTCTGCGCCGCACAGTCCCACCTGCAGTTCCAGGAATCGTGTTCCTCTCAGGCGGACAGAGTGAAGAGGAAG
CAACACTAAATCTGAACGCAATGAACAAGCTCGATGTGTTGAAGCCATGGACTCTCACTTTCTCATTTGGCCGAGCCCTC
CAACAAAGCACTCTCAAGGCTTGGGCAGGTAAGACAGAGAATGTAGCCAAAGCTCAGGCCACTTTCCTGACCAGGTGCAA
GGGTAACTCGGACGCTACCCTCGGGAAATACACCGGCGGGGCTTCTGGTGACTCGGCCGCCTCTGAGAGCTTGTATGAGG
AAGGATACAAGTATTAGGAGCGTTTAAATACGGGTGTCGCCTTTTATACGATTTGAATATATGTCAAATGTTTCGTAGGC
GTTTAACTGTTTAAATTTTTATCGATTTGGTTTAGCGTCTGTGTAATGTTCTTAAACTGTGTTGTGTTTTTTGTGATGGT
TTCTATAATATTTTCGCGCC
>4909806_protein_ID_4909808
MSAFVGKYADELIKTAKYIATPGKGILAADESTGTIGKRFASINVENIESNRQALRELLFTSPGTFPCLSGVILFEETLY
QKTTDGKPFVELLMENGVIPGIKVDKGVVDLAGTNGETTTQGLDSLGARCQEYYKAGARFAKWRAVLKIGATEPSELSIQ
ENAKGLARYAIICQENGLVPIVEPEVLTDGSHDIKKCAAVTETVLAAVYKALNDHHVLLEGTLLKPNMVTPGSDSPKVAP
EVIAEYTVTALRRTVPPAVPGIVFLSGGQSEEEATLNLNAMNKLDVLKPWTLTFSFGRALQQSTLKAWAGKTENVAKAQA
TFLTRCKGNSDATLGKYTGGASGDSAASESLYEEGYKY*
>4949423_construct_ID_YP0096
AACAAATACTAATCATTCTTTCTTACGATTTCTTTAGTAAAATAAGAATATTTCTTGTATATTTCAACCATAAGTAGATA
TGTCTAAATTTAACACTCAGATTACTACATTGTTCATTGTTTTAGCTTTGGTGTGTGCGTTTGTTCCGGCTTTCTCAGTC
GAAGAAGCTGAAGCAACATTATTATGGAATACTTGTCTTGTTAAAATCACTCCTAAGTGTGCTTTGGATATAATCGCTGC
TGTCTTTGAAAATGGAACCATGCCTGATCCTTGTTGCAAGGATCTCGTCAAAGAAGGAAAAGTGTGTCACGATACGCTTA
TTAAATATATTGCAGATAAACCCATGTTAATTGCCCACGAAACAGAATACTTGAAGAAGAGTGATGACTTGTGGAAACAT
TGTGTCTCAATTTCCAAAAGTGCTTCAAATATGGAATGCTTTTACTATTTTGATTTTTGAGCCAAAAAATTGATATTTTC
TGT
>4949423_protein_ID_4949424
MSKFNTQITTLFIVLALVCAFVPAFSVEEAEATLLWNTCLVKITPKCALDIIAAVFENGTMPDPCCKDLVKEGKVCHDTL
IKYIADKPMLIAHETEYLKKSDDLWKHCVSISKSASNMECFYYFDF*
>5787483_construct_ID_YP0180
AACGCCACAATCATGGCTTTGTTCTTATCTCCTAAAACCATCACTCTTCTCTTCTTCTCCCTCTCCCTCGCACTCTACTG
CAGCATCGATCCTTTCCACCACTGCGCCATTTCCGATTTCCCCAATTTCGTCTCTCACGAAGTTATCTCTCCACGTCCCG
ACGAAGTTCCATGGGAGAGAGATTCACAAAATTCACTTCAGAAATCAAAGATTCTGTTTTTTAACCAAATCCAAGGTCCA
GAGAGCGTCGCCTTTGATTCTCTCGGACGTGGTCCGTACACAGGCGTTGCTGATGGTAGGGTTTTGTTTTGGGATGGAGA
GAAATGGATTGATTTCGCTTATACTTCGAGTAATCGATCGGAGATTTGTGATCCGAAGCCTTCTGCTTTGAGTTACTTGA
GGAATGAACATATATGTGGTCGTCCTTTAGGTCTTCGTTTCGATAAGAGAACCGGAGATTTGTATATAGCTGATGCTTAT
ATGGGACTTTTGAAAGTTGGTCCTGAAGGTGGTTTAGCAACGCCGCTTGTAACTGAAGCTGAAGGTGTGCCGTTGGGGTT
TACTAATGATCTTGACATTGCTGATGATGGAACTGTTTACTTTACAGATAGCAGCATTAGTTACCAGAGGAGGAACTTCT
TGCAGCTCGTTTTCTCTGGAGACAATACTGGGAGGGTTCTAAAGTATGATCCAGTAGCTAAGAAAGCTGTTGTTTTGGTC
TCAAATCTTCAGTTTCCGAATGGTGTCTCTATCAGCAGAGACGGTTCTTTCTTTGTATTCTGCGAAGGAGATATTGGAAG
CCTACGAAGATACTGGTTGAAAGGCGAGAAAGCTGGAACGACAGATGTGTTTGCGTATTTACCAGGGCATCCTGATAACG
TAAGAACCAACCAAAAGGGTGAATTTTGGGTAGCGCTTCATTGCAGACGCAACTACTACTCATACTTAATGGCAAGATAT
CCTAAGCTGAGGATGTTCATACTGAGACTGCCAATCACTGCGAGAACTCACTACTCGTTCCAGATAGGGTTACGGCCGCA
CGGGTTGGTGGTTAAGTATAGTCCTGAAGGGAAGCTTATGCATGTTTTGGAAGATAGTGAAGGGAAAGTTGTGAGATCAG
TAAGTGAAGTGGAAGAAAAAGATGGGAAGCTTTGGATGGGAAGTGTGTTGATGAACTTTGTTGCTGTCTATGACCTCTGA
TTACTTGACCTATACGTAAACCACTTCACTCAGTTTCTAGATTTAGCAAATTCCCAAAACTGTTAGGTGTGTACTGAAAA
AATCAAACACTTAGCACAAACAAACTCAATGTTATT
>5787483_protein_ID_5787485
MALFLSPKTITLLFFSLSLALYCSIDPFHHCAISDFPNFVSHEVISPRPDEVPWERDSQNSLQKSKILFFNQIQGPESVA
FDSLGRGPYTGVADGRVLFWDGEKWIDFAYTSSNRSEICDPKPSALSYLRNEHICGRPLGLRFDKRTGDLYIADAYMGLL
KVGPEGGLATPLVTEAEGVPLGFTNDLDIADDGTVYFTDSSISYQRRNFLQLVFSGDNTGRVLKYDPVAKKAVVLVSNLQ
FPNGVSISRDGSFFVFCEGDIGSLRRYWLKGEKAGTTDVFAYLPGHPDNVRTNQKGEFWVALHCRRNYYSYLMARYPKLR
MFILRLPITARTHYSFQIGLRPHGLVVKYSPEGKLMHVLEDSEGKVVRSVSEVEEKDGKLWMGSVLMNFVAVYDL*
>6795099_construct_ID_YP0095
ATGGCCACTGGTGTTTCTGTTGAGAACATAAACCCCAAGGTTATACTAGGGCCATCATCGATCGCTGAGTGCATAGTCAT
TCGTGGAGAGGTTGCCATCCATGCTCAGCACCTACAACAGCAGCTACAGACACAACCTGGTTCTCTTCCATTTGATGAGA
TCGTGTATTGCAACATCGGGAACCCTCAGTCCTTGGGTCAAAAACCAATCACATTCTTCAGGGAGGTTCTTGCACTTTGC
AATCATCCAAATCTGCTGGAGAGAGAGGAAATTAAATCATTGTTCAGCACTGATGCTATTGCTCGGGCAAAGAAAATTCT
TTCCATGATTCCTGGAAGAGCCACCGGGGCATATAGTCATAGCCAGGGTATCAAGGGACTGCGTGATGAGATTGCTGCTG
GGATTGCCTCCCGTGATGGTTTCCCTGCAAATGCAGATGATATATTCCTAACTAATGGAGCAAGTCCTGGTGTACACATG
ATGATGCAGTTGCTGATAAGGAACAACAGAGATGGCATTATGTGTCCAATTCCTCAATACTCATTGTACTCAGCATCCCT
AGCACTTCATGGCGGAGCTCTTGTGCCATATTATCTTGATGAATCCTCAGGATGGGGTTTGGAGGTTTCTAAGCTTAAGA
ATCAACTTGAAGATGCCAGGTCAAAAGGCATAACTGTTAGGGCGTTGGTGGTGATCAATCCTGGAAATCCTACTGGACAG
ATTCTTGATGAGCAACAGCAATATGAGCTAGTAAAGTTCTGCAAGGACGAGGAACTTGTTCTTCTGGCGGATGAGGTATA
CCAAGAGAACATTTATGTTACCAACAAGAAGATCAACTCTTTCAAGAAGATAGCAAGATCCATGGGATACAATGGAGACG
ATTTACAATTAGTATCATTGCATTCTGTTTCTAAAGGATATTACGGAGAGTGTGGCAAGAGAGGCGGTTACATGGAGGTC
ACTGGCTTCAGCACTCCAGTTAGAGAACAACTCTACAAAATTGCATCTGTTAACTTGTGTTCAAATATCACCGGCCAGAT
CCTTGCGAGCCTCATAATGGATCCACCAAAGGCTGGGGACGCATCTTATGACCTCTACGAGGAAGAGAAAGACAACATCC
TAAAATCTTTATCTCGTCGTGCAAAGGCAATGGAGTCTGCATTTAACAGTATTGATGGAATTACATGCAACAAGACGGAA
GGGGCGATGTATCTGTTCCCACGGATTTATCTACCACAGAAGGCAATTGAGGCTGCCAGGGCTGTCAACAAAGCACCTGA
TGTATTCTACGCTCTACGTCTTCTTGATACCACCGGCATCGTTGTGACTCCTGGATCTGGTTTTGGACAAGTTGCAGGGA
CATGGCACGTGAGATGCACGATCCTGCCGCAGGAGGAGAAGATACCTTCGATGATCTCCCGCTTCAGGGAATTCCATGAG
GAGTTCATGTCACAGTATCGCGACTGA
>679S099_protein_ID_6795100
MATGVSVENINPKVILGPSSIAECIVIRGEVAIHAQHLQQQLQTQPGSLPFDEIVYCNIGNPQSLGQKPITFFREVLALC
NHPNLLEREEIKSLFSTDAIARAKKILSMIPGRATGAYSHSQGIKGLRDEIAAGIASRDGFPANADDIFLTNGASPGVHM
MMQLLIRNNRDGIMCPIPQYSLYSASLALHGGALVPYYLDESSGWGLEVSKLKNQLEDARSKGITVRALVVINPGNPTGQ
ILDEQQQYELVKFCKDEELVLLADEVYQENIYVTNKKINSFKKIARSMGYNGDDLQLVSLHSVSKGYYGECGKRGGYMEV
TGFSTPVREQLYKIASVNLCSNITGQILASLIMDPPKAGDASYDLYEEEKDNILKSLSRRAKAMESAFNSIDGITCNKTE
GAMYLFPRIYLPQKAIEAARAVNKAPDVFYALRLLDTTGIVVTPGSGFGQVAGTWHVRCTILPQEEKIPSMISRFREFHE
EFMSQYRD*
>12321680_construct_ID_YP0112
ATATTCTTAGTACAAATAAGAAATTCACACCCCTCAAAGAAATATAACATAATCAATCATAGGAAATATACTTCGCATAA
TGACGATAATGATCAAGTTTCTCCTGTTAGCTCTGCTCGTGATCTCTCCGATTTGCGCCGAGAAGGACCTGATGAAAGAG
GAATGCCATAATGCACAAGTTCCGACCATTTGCATGCAATGTCTTGAATCCGACCCAACCTCCGTTCATGCAGACCGTGT
TGGCATCGCCGAGATCATCATACACTGTCTCGACTCTCGTCTCGATATCATCACCAATAACATTACAAATATATTGTCAC
TGGGAGGAGGAACGAAAGAAGTGAGAAAAATCTTGGAGGATTGCAGAAATGACACGTCGACGGTGGCACCTAAACTACTG
TCGGAAGCCAAAACAGGTCTGAAAACCGGTGATTACGACAAAGCCGCCAAATCGATAGAGTATGCTAGCATTCCTCATAG
CTGTGGATTAAAGCAACCAAGTGTCGAGTTTGAGTTTCTTCAACTGTTTAGTCAAATCAGTATCTATACTCAACTCTCTG
ATGCTGCCATGAGAATCATTGATCGCTTCTAATTACTCCACCTTTTTATCTCTATGTAACTCAACAACATCGATGCTTAC
CATGCATCCCCCATATAAATAAATGATTCCCTCTTTTA
>12321680_protein_ID_12321681
MTIMIKFLLLALLVISPICAEKDLMKEECHNAQVPTICMQCLESDPTSVHADRVGIAEIIIHCLDSRLDIITNNITNILS
LGGGTKEVRKILEDCRNDTSTVAPKLLSEAKTGLKTGDYDKAAKSIEYASIPHSCGLKQPSVEFEFLQLFSQISIYTQLS
DAAMRIIDRF*
>12325134_construct_ID_YP0116
AACTCAACTCACTCAAACCAAAAAAAGAAACATCAAACCCTAPAACACACATAACAATCACAAATGAAGAATCCTTCAGT
GATCTCTTTTCTCATCATTCTCCTGTTTGCTGCAACTATTTGCACCCACGGAAATGAACCGGTGAAGGATACAGCCGGAA
ATCCACTTAACACCCGCGAACAATACTTCATCCAGCCGGTTAAGACCGAGAGTAAAAACGGAGGTGGTCTTGTCCCAGCC
GCCATTACAGTACTTCCCTTTTGTCCACTTGGCATCACCCAAACACTTCTTCCCTACCAACCCGGCCTACCGGTTAGCTT
CGTATTAGCACTTGGCGTAGGATCAACCGTTATGACATCTTCGGCTGTAAACATCGAGTTCAAGTCCAACATCTGGCCGT
TTTGCAAGGAGTTTTCCAAGTTTTGGGAAGTTGATGATTCCTCATCAGCTCCCAAGGAGCCTTCAATTCTCATCGGTGGT
AAAATGGGGGACCGAAATAGCTCGTTTAAGATTGAGAAAGCTGGAGAAGGAGCTAGAGCAAACGTTTATAAGTTGACCAC
CTTTTACGGAACCGTTGGAGCCATCCCAGGGGTTTGGTTAAGCGCACCACAACTAATTATCACCAAGGATACGGCTAAGA
CCTTACTCGTCAAATTCAAAAAGGTTGATGATGCTACTACGGCTACTAGCAACTTATACTTCCCGGGTTGATAATTTAGG
TCTAAGGATGTTCCCGTTCTACTAATCAACTGGTAAAAATTATTGTAATATTAAGCCTGAGACTCGTCCATGGCCTAAAA
TAATGAGTTATTTTCAAATTTCAATTAATAAGAAAGAAAAATGTGGCCAGATCCAGATACATAGATGTTGAGAATCATTC
ATAGGCATTGCTGTTGAATCTGTTTAAGGCATGAAATAGTTTTCTTCTTCATTCTACTTTGTATCCGAAAATTTTCTCTC
CTCTTGTAAAGATCTTGAGCTTGAGAAAACATTGATCATTCAT
>12325134_protein_ID_12325135
MKNPSVISFLIILLFAATICTHGNEPVKDTAGNPLNTREQYFIQPVKTESKNGGGLVPAAITVLPFCPLGITQTLLPYQP
GLPVSFVLALGVGSTVMTSSAVNIEFKSNIWPFCKEFSKFWEVDDSSSAPKEPSILIGGKMGDRNSSFKIEKAGEGARAN
VYKLTTFYGTVGAIPGVWLSAPQLIITKDTAKTLLVKFKKVDDATTATSNLYFPG*
>12329827_construct_ID_YP0118
AATCATCATCCAAAAACATTCTTCTCACAAGAATCAGATTCAAGATAGAAGTTTTTCAAACAATGTCTAGTCCTCTTGGT
CACTTTCAGATTCTTGTTTTTCTTCATGCTTTGCTTATCTTCTCAGCTGAGTCCCGCAAAACCCAATTGCTGAACGATAA
TGATGTTGAATCTAGCGACAAGAGTGCAAAAGGCACACGATGGGCTGTTTTAGTTGCTGGATCAAATGAATATTATAACT
ACAGGCATCAGGCTGACATATGCCACGCGTATCAGATACTCCGAAAAGGCGGTTTAAAAGATGAAAACATCATTGTGTTT
ATGTATGATGATATCGCGTTTTCCTCGGAGAATCCTAGGCCTGGAGTTATCATTAATAAACCAGATGGAGAAGATGTTTA
TAAAGGAGTTCCTAAGGACTACACTAAAGAAGCTGTTAATGTTCAAAACTTCTACAATGTGTTACTTGGAAATGAAAGTG
GCGTCACAGGAGGAAATGGCAAAGTTGTGAAAAGTGGTCCTAATGATAATATCTTCATCTATTATGCTGACCATGGAGCT
CCTGGCTTAATAGCGATGCCCACTGGTGATGAAGTTATGGCAAAAGATTTCAATGAAGTCTTGGAGAAGATGCATAAGAG
AAAAAAATACAACAAGATGGTGATCTATGTTGAAGCATGTGAATCAGGAAGTATGTTTGAAGGGATTTTAAAGAAAAATC
TCAACATATACGCAGTGACTGCTGCTAATTCTAAAGAGAGCAGCTGGGGAGTTTACTGTCCTGAGTCATATCCTCCTCCT
CCTTCTGAGATTGGAACTTGTCTCGGCGATACATTTAGCATCTCTTGGCTTGAGGACAGTGACCTTCATGACATGAGCAA
AGAGACTTTGGAGCAACAATACCACGTTGTAAAGAGAAGAGTAGGATCTGATGTACCAGAGACTTCTCATGTATGCCGTT
TCGGAACAGAGAAGATGCTTAAAGATTATCTTTCCTCTTACATTGGAAGAAATCCTGAAAACGATAACTTCACTTTCACG
GAATCCTTTTCCTCACCAATCTCTAATTCTGGCTTGGTCAATCCGCGCGATATTCCTCTGCTATACCTCCAGAGAAAGAT
TCAAAAAGCTCCAATGGGATCACTTGAAAGCAAAGAAGCTCAGAAGAAATTGCTTGACGAAAAGAATCATAGGAAACAAA
TCGATCAGAGCATTACAGACATTCTGCGGCTTTCAGTTAAACAAACCAATGTCTTAAATCTCTTAACTTCCACAAGAACA
ACAGGACAGCCTCTTGTAGACGATTGGGATTGCTTCAAGACTCTAGTTAATAGCTTCAAGAATCACTGCGGTGCAACGGT
GCATTACGGATTGAAGTATACAGGAGCGCTTGCCAATATCTGCAATATGGGAGTGGATGTGAAGCAAACTGTTTCAGCCA
TTGAACAAGCTTGTTCGATGTAATGATTTGCAAAACAATGTGATATTCGACTTTAAAAATATCAAAGTTAATTTCAATAA
AACTCGATGTAGAGATGGTTGGTTCATGATACTACTTTTACAT
>12329827_protein_ID _2329829
MSSPLGHFQILVFLHALLIFSAESRKTQLLNDNDVESSDKSAKGTRWAVLVAGSNEYYNYRHQADICHAYQILRKGGLKD
ENIIVFMYDDIAFSSENPRPGVIINKPDGEDVYKGVPKDYTKEAVNVQNFYNVLLGNESGVTGGNGKVVKSGPNDNIFIY
YADHGAPGLIANPTGDEVMAKDFNEVLEKMHKRKKYNKMVIYVEACESGSMFEGILKKNLNIYAVTAANSKESSWGVYCP
ESYPPPPSEIGTCLGDTFSISWLEDSDLHDMSKETLEQQYHVVKRRVGSDVPETSHVCRFGTEKMLKDYLSSYIGRNPEN
DNFTFTESFSSPISNSGLVNPRDIPLLYLQRKIQKAPMGSLESKEAQKKLLDEKNHRKQIDQSITDILRLSVKQTNVLNL
LTSTRTTGQPLVDDWDCFKTLVNSFKNHCGATVHYGLKYTGALANICNMGVDVKQTVSAIEQACSM*
>12332135_construct_ID_YP0113
ATCACCACCACCAAATATCAAACGCAAAAACCTATTATCAAAAGAACTAGGGAGAAATGACTAATCCCATGATCATGGTT
ATGCTGTTGTTGTTTCTTGTGATGTCGACTAGAGCAGACGAAGAGCTGATTAAGACAGAGTGTAATCACACAGAATACCA
AAACGTATGCCTCTTCTGTCTTGAAGCCGATCCAATCTCCTTCAATATCGACCGTGCTGGACTTGTCAACATCATTATAC
ACTGTCTCGGATCTCAACTTCATGTTCTTATCAACACCGTCACGAGTCTAAAGTTGATGAAAGGAGAGGGTGAAGCAAAT
GAGAATGTTCTGAAAGATTGCGTCACAGGCTTTGCGATTGCACAATTACGACTTCAAGGAGCCAACATCGATTTGATAAC
CCTTAATTACGATAAAGCGTACGAATTGGTGAAAACTGCGTTAAACTATCCTCGGACTTGCGAAGAAAATCTCCAAAAAC
TCAAGTTCAAAGATTCATCTGATGTTTATGACGATATCTTGGCATATAGCCAACTCACCTCTGTTGCTAAGACGTTGATC
CACCGTCTCTAGATCAATATATATGTCGATCTGGTTATCAAAAATATATTTATGTCGATCGTTTGCTACCACTAATAAAA
TAAAACTCCATTATGTATGTCACGCGTGATTTAATTTCACTCATCAACAAATAAAATAAAATAAAATAAAATGTTTAG
>12332135_protein_ID_12332136
MTNPMIMVMLLLFLVMSTRADEELIKTECNHTEYQNVCLFCLEADPISFNIDRAGLVNIIIHCLGSQLDVLINTVTSLKL
MKGEGEANENVLKDCVTGFAIAQLRLQGANIDLITLNYDKAYELVKTALNYPRTCEENLQKLKFKDSSDVYDDILAYSQL
TSVAKTLIHRL*
>12333534_construct_ID_YP0138
CACCCATCTCCTTCTCCATAACTCTCTCTCTCTCTCCCTAJACACAACCAAAGACTTTTATCTCTCAGGAACCCCAAAAA
CAAATGGCTATAATGAAGAAAACTTCAAAACTCACTCAAACAGCAATGCTGAAGCAGATTCTGAAGAGATGCTCGAGCTT
AGGGAAGAAGAATGGAGGAGGGTACGATGAAGATTGCCTTCCGCTTGACGTACCAAAGGGACACTTCCCTGTCTATGTCG
GAGAGAACAGAAGCAGATACATTGTCCCAATCTCCTTCTTGACACATCCTGAGTTCCAATCTCTCTTACAACGAGCCGAG
GAAGAATTTGGATTCGATCACGACATGGGTCTCACCATTCCTTGTGATGAACTCGTTTTTCAAACCCTAACATCCATGAT
CCGATGATATTTTATCATTTGAAGAAGAAGCAGAAGGAGATGGTTAAAGAAGAAGCGGAAAAGCTTCTCATACAAAAAAA
GCATCTCTTCTCTTTTTTTAAGATTTTTTTTCCTTTATTTTTAAGCCCATCTAGGGTTTTTTTTACGAGTTAATTGACTC
GTCTAACTAGAAATAAATCCGTATGAGATAGAGATTCTATGGGTTTAGATCTGTAAATAAAGTTTGTAATGTTTTCCTCA
CAGATCTTCGTTCTGTGAGAGAAGTTATTTAATGCAAGAGAAAGTATTCCTCC
>12333534_protein_ID_12333535
MAIMKKTSKLTQTAMLKQILKRCSSLGKKNGGGYDEDCLPLDVPKGHFPVYVGENRSRYIVPISFLTHPEFQSLLQRAEE
EFGFDHDMGLTIPCDELVFQTLTSMIR*
>12348737_construct_ID_YP0054
ATTTTGGTTAAAGCAAAAGATTTTAAGAGAGAAAGGGGGAGAAGTGAGAGAGATGGAGCATAAGAGAGGACATGTATTAG
CAGTGCCGTACCCAACGCAAGGACACATCACACCATTCCGCCAATTCTGCAAACGACTTCACTTCAAAGGTCTCAAAACC
ACTCTCGCTCTCACCACTTTCGTCTTCAACTCCATCAATCCTGACCTATCCGGTCCAATCTCCATAGCCACCATCTCCGA
TGGCTATGACCATGGGGGTTTCGAGACAGCTGACTCCATCGACGACTACCTCAAAGACTTTAAAACTTCCGGCTCGAAAA
CCATTGCAGACATCATCCAAAAACACCAGACTAGTGATAACCCCATCACTTGTATCGTCTATGATGCTTTCCTGCCTTGG
GCACTTGACGTTGCTAGAGAGTTTGGTTTAGTTGCGACTCCTTTCTTTACGCAGCCTTGTGCTGTTAACTATGTTTATTA
TCTTTCTTACATAAACAATGGAAGCTTGCAACTTCCCATTGAGGAATTGCCTTTTCTTGAGCTCCAAGATTTGCCTTCTT
TCTTCTCTGTTTCTGGCTCTTATCCTGCTTACTTTGAGATGGTGCTTCAACAGTTCATAAATTTCGAPAAAGCTGATTTC
GTTCTCGTTAATAGCTTCCAAGAGTTGGAACTGCATGAGAATGAATTGTGGTCGAAAGCTTGTCCTGTGTTGACAATTGG
TCCAACTATTCCATCAATTTACTTAGACCAACGTATCAAATCAGACACCGGCTATGATCTTAATCTCTTTGAATCGAAAG
ATGATTCCTTCTGCATTAACTGGCTCGACACAAGGCCACAAGGGTCGGTGGTGTACGTAGCATTCGGAAGCATGGCTCAG
CTGACTAATGTGCAGATGGAGGAGCTTGCTTCAGCAGTAAGCAACTTCAGCTTCCTGTGGGTGGTCAGATCTTCAGAGGA
GGAAAAACTCCCATCAGGGTTTCTTGAGACAGTGAATAAAGAAAAGAGCTTGGTCTTGAAATGGAGTCCTCAGCTTCAAG
TTCTGTCAAACAAAGCCATCGGTTGTTTCTTGACTCACTGTGGCTGGAACTCAACCATGGAGGCTTTGACCTTCGGGGTT
CCCATGGTGGCAATGCCCCAATGGACTGATCAACCGATGAACGCAAAGTACATACIAGATGTGTGGAAGGCTGGAGTTCG
TGTGAAGACAGAGAAGGAGAGTGGGATTGCCAAGAGAGAGGAGATTGAGTTTAGCATTAAGGAAGTGATGGAAGGAGAGA
GGAGCAAAGAGATGAAGAAGAACGTGAAGAAATGGAGAGACTTGGCTGTCAAGTCACTCAATGAAGGAGGTTCTACGGAT
ACTAACATTGATACATTTGTATCAAGGGTTCAGAGCAAATAGGTAACTCACATACAGTAGCAAAGGTCCTTCTATAATAT
CTTGTTTTGTACGTCTTTCATTCAGCATAATCTTTTGTTGACTTTTCTTATGTTGTATGTTCAAATCCCCATATTGCTTC
TTGTTGTATGTTCAAATCCCCATATTGCTTCTTGTTGACAATAATAATAATAAAAACAATGCAACTTTACC
>12348737_protein_ID_12348739
MEHKRGHVLAVPYPTQGHITPFRQFCKRLHFKGLKTTLALTTFVFNSINPDLSGPISIATISDGYDNGGFETADSIDDYL
KDFKTSGSKTIADIIQKHQTSDNPITCIVYDAFLPWALDVAREFGLVATPFFTQPCAVNYVYYLSYINNGSLQLPIEELP
FLELQDLPSFFSVSGSYPAYFEMVLQQFINFEKADFVLVNSFQELELHENELWSKACPVLTIGPTIPSIYLDQRIKSDTG
YDLNLFESKDDSFCINWLDTRPQGSVVYVAFGSMAQLTNVQMEELASAVSNFSFLWVVRSSEEEKLPSGFLETVNKEKSL
VLKWSPQLQVLSNKAIGCFLTHCGWNSTMEALTFGVPMVANPQWTDQPMNAKYIQDVWKAGVRVKTEKESGIAKREEIEF
SIKEVMEGERSKEMKKNVKKWRDLAVKSLNEGGSTDTNIDTFVSRVQSK*
>12370148_construct_ID_YP0033
ATTCCCACTTCCACACATACACATATACAACAGAGCAAGAGAGTCAATCAAGTAGAGTGAAGATGGCAACTAAACAAGAA
GCTTTAGCCATCGATTTCATAAGCCAACACCTTCTCACAGACTTTGTTTCCATGGAAACTGATCACCCATCTCTTTTTAC
CAACCAACTTCACAACTTTCACTCAGAAACAGGCCCTAGAACCATCACCAACCAATCCCCTAAACCGAATTCGACTCTTA
ACCAGCGTAAACCGCCCTTACCGAATCTATCCGTCTCGAGAACGGTTTCAACAAAGACAGAGAAAGAGGAAGAAGAGAGG
CACTACAGGGGAGTGAGACGAAGACCGTGGGGAAAATACGCGGCGGAGATTAGGGATCCGAACAAAAAGGGTTGTAGGAT
CTGGCTTGGGACTTACGACACTGCCGTGGAAGCTGGAAGAGCTTATGACCAAGCGGCGTTTCAATTACGTGGAAGAAAAG
CAATCTTGAATTTCCCTCTCGATGTTAGGGTTACGTCAGAAACTTGTTCTGGGGAAGGAGTTATCGGATTAGGGAAACGA
AAGCGAGATAAGGGTTCTCCGCCGGAAGAGGAGAAGGCGGCTAGGGTTAAAGTGGAGGAAGAAGAGAGTAATAcGTCGGA
GACGACGGAGGCTGAGGTTGAGCCGGTGGTACCATTGACGCCGTCAAGTTGGATGGGGTTTTGGGATGTGGGAGCAGGAG
ATGGTATTTTCAGTATTCCTCCGTTATCTCCGACGTCTCCCAACTTTTCCGTTATCTCCGTCACTTAAAACTTCGGAAAA
GTCAACGTACGATGACGTTTTCACTTGCGTCACTCTCATGATTTCATTTATTCTTGTATAATATAAAGGTAGCGGTAGTG
TGCAAATATCAAATAAGTAGTTTAATTAGTACCAATCATTTTATTCATTATTTTTTTTAGTAGAATATTTGGATGTTGAA
AATATAAATTTAATTTTGTATTTGTTGATGTTATAAATTTATTGATTGTATAAACATTCTTAGTC
>12370148_protein_ID_2370150
MATKQEALAIDFISQHLLTDFVSMETDHPSLFTNQLHNFHSETGPRTITNQSPKPNSTLNQRKPPLPNLSVSRTVSTKTE
KEEEERHYRGVRRRPWGKYAAEIRDPNKKGCRIWLGTYDTAVEAGRAYDQAAFQLRGRKAILNFPLDVRVTSETCSGEGV
IGLGKRKRDKGSPPEEEKAARVKVEEEESNTSETTEAEVEPVVPLTPSSWMGFWDVGAGDGIFSIPPLSPTSPNFSVISV
T*
>12396394_construct_ID_YP0056
GGTCCCAAAGAAAAATACGCACACCTACTCCCTTCATTCTCTATCCTCTCCACTCATAATATATACATCTAAATGCAATC
TCTCCAATTTGCACCCAATTTCTTCGAATCAACTTATCAATGGCCTCATCAGCTGCGATGTTCATGCTCCCTCTTCCTCT
AACTCAGCAGATAACAACAAACAATACTCTGCAGACTACAGCCACACCGGAACCGTCAGCCTCCATAGTTAAATGCCTTT
TTCCGGCGAGAAACTCATCGGAAAGTTCTGCTCGTTCGAAGTTTAGTCTTTGGCTATTTGGCAATCCCGCTACGTATGAC
AAGAGGTTCCAAGAAGCTATTGAACTTAGTTGCTTGTGATGGAGATTTGGAGATTTTTCCTAGTCTTTTTCTTGTGTTTT
TTAAATGGACATATTGTAATTTCTTCCCAAGTCTCACCCTCCGCTGTAATTTATCTAATAATCAATTCGATCAAAGATGT
TCCGACTG
>12396394_protein_ID_12396395
MASSAAMIFMLPLPLTQQITTNNTLQTTATPEPSASIVKCLFPARNSSESSARSKFSLWLFGNPATYDKRFQEAIELSCL*
>12561142_construct_ID_YP0028
ATGGATACTCTCTTTAGACTAGTCAGTCTCCAACAACAACAACAATCCGATAGTATCATTACAAATCAATcTTCGTTAAG
CAGAACTTCCACCACCACTACTGGCTCTCCACAAACTGCTTATCACTACAACTTTCCACAAAACGACGTCGTCGAAGAAT
GCTTCAACTTTTTCATGGATGAAGAAGACCTTTCCTCTTCTTCTTCTCACCACAACCATCACAACCACAACAATCCTAAT
ACTTACTACTCTCCTTTCACTACTCCCACCCAATACCATCCCGCCACATCATCAACCCCTTCCTCCACCGCCGCAGCCGC
AGCTTTAGCCTCGCCTTACTCCTCCTCCGGCCACCATAATGACCCTTCCGCGTTCTCCATACCTCAAACTCCTCCGTCCT
TCGACTTCTCAGCCAATGCCAAGTGGGCAGACTCGGTCCTTCTTGAAGCGGCACGTGCCTTCTCCGACAAAGACACTGCA
CGTGCGCAACAAATCCTATGGACGCTCAACGAGCTCTCTTCTCCGTACGGAGACACCGAGCAAAAACTGGCTTCTTACTT
CCTCCAAGCTCTCTTCAACCGCATGACCGGTTCAGGCGAACGATGCTACCGAACCATGGTAACAGCTGCAGCCACAGAGA
AGACTTGCTCCTTCGAGTCAACGCGAAAAACTGTACTAAAGTTCCAAGAAGTTAGCCCCTGGGCCACGTTTGGACACGTG
GCGGCAAACGGAGCAATCTTGGAAGCAGTAGACGGAGAGGCAAAGATCCACATCGTTGACATAAGCTCCACGTTTTGCAC
TCAATGGCCGACTCTTCTAGAAGCTTTAGCCACAAGATCAGACGACACGCCTCACCTAAGGCTAACCACAGTTGTCGTGG
CCAACAAGTTTGTCAACGATCAAACGGCGTCGCATCGGATGATGAAAGAGATCGGAAACCGAATGGAGAAATTCGCTAGG
CTTATGGGAGTTCCTTTCAAATTTAACATTATTCATCACGTTGGAGATTTATCTGAGTTTGATCTCAACGAACTCGACGT
TAAACCAGACGAAGTCTTGGCCATTAACTGCGTAGGCGCGATGCATGGGATCGCTTCACGTGGAAGCCCTAGAGACGCTG
TGATATCGAGTTTCCGACGGTTAAGACCGAGGATTGTGACGGTCGTAGAAGAAGAAGCTGATCTTGTCGGAGAAGAAGAA
GGTGGCTTTGATGATGAGTTCTTGAGAGGGTTTGGAGAATGTTTACGATGGTTTAGGGTTTGCTTCGAGTCATGGGAAGA
GAGTTTTCCAAGGACGAGCAACGAGAGGTTGATGCTAGAGCGTGCAGCGGGACGTGCGATCGTTGATCTTGTGGCTTGTG
AGCCGTCGGATTCCACGGAGAGGCGAGAGACAGCGAGGAAGTGGTCGAGGAGGATGAGGAATAGTGGGTTTGGAGCGGTG
GGGTATAGTGATGAGGTGGCGGATGATGTCAGAGCTTTGTTGAGGAGATATAAAGAAGGTGTTTGGTCGATGGTACAGTG
TCCTGATGCCGCCGGATATTCCTTTGTTGGAGAGATCAGCCGGTGGTTTGGGCTAGTGCGTGGCGGCCAAACGTAAAGGG
TTGTTTTTATTTTTTCATAAGGAATTCGCAAGTTCGATTTTTACTTGAGATGGTTTCACACGTGTGGTGATGGTTGATGA
TGGGCTTTGAGATTGAGAGAGTTACGATTATGATGATAATGCAGTTCATAATATGATTTTTGGATTTGGTTTAGGACTAA
TTAAGTAATTCTGATCATTGAGGTGGGTATCAAGGTTCATACAATTCGTGATTTTTTGTTTTGTCTTTGGTATTTATTAA
TTTTAAAAATCCATTTTGGAATGAAATTTGTGATTACTTTTGTTTATCCG
>12561142_protein_ID_12561143
MDTLFRLVSLQQQQQSDSIITNQSSLSRTSTTTTGSPQTAYHYNFPQNDVVEECFNFFMDEEDLSSSSSHHNHHNHNNPN
TYYSPFTTPTQYHPATSSTPSSTAAAAALASPYSSSGHHNDPSAFSIPQTPPSFDFSANAKWADSVLLEAARAFSDKDTA
RAQQILWTLNELSSPYGDTEQKLASYFLQALFNRMTGSGERCYRTMVTAAATEKTCSFESTRKTVLKFQEVSPWATFGHV
AANGAILEAVDGEAKIHIVDISSTFCTQWPTLLEALATRSDDTPHLRLTTVVVANKFVNDQTASHRMMKEIGNRMEKFAR
LMGVPFKFNIIHHVGDLSEFDLNELDVKPDEVLAINCVGAMHGIASRGSPRDAVISSFRRLRPRIVTVVEEEADLVGEEE
GGFDDEFLRGFGECLRWFRVCFESWEESFPRTSNERLMLERAAGRAIVDLVACEPSDSTERRETARKWSRRMRNSGFGAV
GYSDEVADDVRALLRRYKEGVWSMVQCPDAAGIFLCWRDQPVVWASAWRPT*
>12576899_construct_ID_YP0020
AACCAAAGACTCTTTACCATCTCTTTCTCTCTCTGTTTGAAGACATAGCACAAAAAAAAAAAAAAAGACAGAGCAAAAAA
ACACACAAAGATGGGCATAATGATGATGATTTTGGGTCTTCTTGTGATCATTGTTTGTTTATGTACTGCTCTTCTCCGAT
GGAACCAGATGCGATATTCTAAGAAAGGTCTTCCTCCTGGAACCATGGGCTGGCCAATATTTGGTGAAACGACTGAGTTT
CTTAAACAAGGACCAGATTTCATGAAAAACCAAAGACTAAGATATGGGAGTTTCTTCAAGTCTCACATTCTTGGTTGCCC
AACAATAGTCTCAATGGACGCAGAGTTAAACATACATACATTCTTTAATGAATCGAAAGGACTTGTTGCCGGTTACCCGC
AATCTATGCTTGATATTCTAGGGACATGCAACATAGCTGCGGTTCATGGCCCGAGCCACCGGCTAATGAGAGGCTCGTTG
CTTTCTTTAATAAGCCCAACCATGATGAAAGACCATCTCTTGCCTAAGATTGATGATTTCATGAGAAACTATCTTTGTGG
TTGGGATGATCTTGAGACAGTTGATATCCAAGAAAAGACCAAACATATGGCATTTTTATCATCGTTGTTACAAATAGCTG
AGACTTTGAAAAAACCAGAGGTTGAAGAATATAGAACAGAGTTTTTCAAGCTTGTTGTGGGAACTCTATCGGTCCCGATC
GATATCCCGGGAACGAATTACCGCAGTGGAGTCCAAGCAAGAAACAACATCGATAGGTTATTGACAGAACTGATGCAAGA
AAGAAAAGAGTCTGGAGAAACTTTCACAGACATGTTGGGTTACTTGATGAAGAAGGAAGATAACCGATACTTGTTAACCG
ATAAAGAGATAAGAGATCAAGTGGTAACGATCTTGTATTCCGGTTATGAGACTGTCTCTACAACCTCCATGATGGCTCTT
AAGTATCTCCATGATCATCCAAAAGCTCTTGAAGAACTCAGAAGAGAACATTTGGCTATAAGGGAGAGAAAACGACCTGA
CGAACCGCTCACTCTCGACGATATTAAATCGATGAAATTCACTCGAGCTGTGATCTTTGAGACATCAAGATTGGCAACGA
TTGTTAATGGTGTCCTTAGGAAAACTACTCACGACTTAGAACTCAACGGTTATTTAATCCCAAAAGGTTGGAGAATTTAC
GTATACACAAGAGAGATTAACTATGATACATCTCTTTATGAAGATCCAATGATCTTTAACCCATGGAGATGGATGGAAAA
GAGCTTAGAATCAAAGAGCTATTTCTTACTCTTTGGAGGTGGAGTTAGGCTTTGCCCTGGAAAGGAACTAGGAATCTCGG
AAGTCTCAAGCTTCCTTCACTACTTTGTTACAAAATATAGATGGGAAGAGAATGGAGAAGACAAATTAATGGTCTTTCCA
AGAGTTTCTGCACCAAAAGGATACCATCTTAAGTGTTCACCTTACTGACTAGTTTTGTCCTAATATTGAAAAATGTGTAA
ATAAATCTATTAAGGGTCATTTTGTAGGGCTAATTAACCTATTTTATCTATTAAATCTCTCAAGATCATAGAGGAGATGG
ATAATGTACAGAGAGAAAGAGAGAAGAAGAAAATGGAATATAGAAAAAAATAAAATATTTGAAATGTTGAGCTTAGTCTC
TTATCTTGTAAATTTGTAACCCATAAATTTTTACATTTCAT
>12576899_protein_ID_12576900
MGIMMMILGLLVIIVCLCTALLRWNQMRYSKKGLPPGTMGWPIFGETTEFLKQGPDFMKNQRLRYGSFFKSHILGCPTIV
SMDAELNRYILMNESKGLVAGYPQSMLDILGTCNIAAVHGPSHRLMRGSLLSLISPTMMKDHLLPKIDDFMRNYLCGWDD
LETVDIQEKTKHMAFLSSLLQIAETLKKPEVEEYRTEFFKLVVGTLSVPIDIPGTNYRSGVQARNNIDRLLTELMQERKE
SGETFTDMLGYLMKKEDNRYLLTDKEIRDQVVTILYSGYETVSTTSMMALKYLHDHPKALEELRREHLAIRERKRPDEPL
TLDDIKSMKFTRAVIFETSRLATIVNGVLRKTTHDLELNGYLIPKGWRIYVYTREINYDTSLYEDPMIFNPWRWMEKSLE
SKSYFLLFGGGVRLCPGKELGISEVSSFLHYFVTKYRWEENGEDKLMVFPRVSAPKGYHLKCSPY
>12646933_construct_ID_YP0121
ATTATATTTTGTTAAGTCCACTCTTCTCTCTCATATCTTCTAACCAAAACAGAGTCACAAGGGGCTCTTAAGCCCTTCCA
ACTAAATTCTTTTCTTTTGTTCTCTTGAAACTGAATCCACCAGACAAAAAAATGGGGGTTGATGGTGAACTGAAAAAGAA
GAAATGCATCATTGCTGGGGTTATCACAGCCTTGCTCGTTCTCATGGTTGTCGCTGTTGGCATCACAACATCAAGAAACA
CCAGTCATTCAGAAAAAATCGTCCCTGTGCAGATTAAAACAGCCACCACGGCAGTTGAAGCAGTTTGTGCACCTACTGAT
TACAAAGAGACTTGTGTCAATAGTCTCATGAAAGCTTCTCCTGACTCTACTCAGCCTCTTGATCTCATTAAGCTTGGCTT
CAACGTCACCATTCGATCCATAGAAGATAGCATCAAGAAAGCTTCCGTGGAGCTGACAGCCAAGGCAGCTAATGACAAGG
ATACCAAAGGGGCTTTGGAGTTGTGTGAGAAGCTTATGAATGATGCTACAGATGATCTGAAGAAGTGTCTTGATAACTTT
GATGGGTTCTCAATTCCTCAGATTGAGGACTTTGTCGAAGATCTTCGTGTTTGGCTTAGTGGCTCCATTGCTTATCAACA
AACATGTATGGATACGTTTGAAGAAACTAACTCGAAACTTTCACAAGACATGCAGAAAATCTTTAAAACATCTAGAGAAC
TCACTAGTAATGGCCTTGCCATGATTACTAACATCTCTAACCTTCTCGGAGAGTTCAACGTCACAGGAGTAACCGGGGAT
CTCGGTAAATACGCAAGAAAACTTTTGTCGGCGGAAGACGGTATACCAAGTTGGGTTGGACCAAACACTAGACGGCTCAT
GGCAACGAAAGGAGGTGTGAAAGCTAACGTGGTGGTTGCACACGACGGAAGTGGTCAGTACAAGACTATCAATGAAGCCT
TGAATGCAGTGCCTAAAGCCAACCAAAAGCCATTTGTTATCTACATTAAGCAAGGTGTCTATAACGAGAAAGTTGACGTC
ACCAAGAAAATGACTCATGTCACTTTCATCGGTGATGGACCAACCAAAACTAAGATCACTGGTAGTCTCAACTATTACAT
TGGCAAGGTCAAGACATACCTTACTGCCACTGTTGCGATCAATGGTGATAACTTCACGGCGAAGAACATCGGGTTTGAAA
ACACTGCAGGTCCCGAAGGACATCAAGCTGTGGCCCTAAGAGTCTCGGCGGATTTGGCCGTCTTCTACAACTGCCAAATC
GATGGTTACCAAGACACACTCTACGTCCATTCTCATCGTCAATTCTTCCGTGACTGCACAGTCTCGGGCACCGTTGACTT
CATTTTCGGCGATGGTATAGTAGTCTTACAAAACTGTAACATTGTTGTGAGAAAACCCATGAAAAGTCAGTCTTGCATGA
TCACAGCCCAAGGCCGCTCCGATAAACGTGAATCCACCGGACTCGTGCTACAAAACTGCCATATTACCGGAGAACCAGCG
TATATTCCCGTAAAATCTATAAACAAAGCATATCTTGGAAGGCCATGGAAAGAGTTTTCAAGAACCATTATAATGGGAAC
AACCATAGACGACGTTATTGATCCAGCGGGATGGCTTCCTTGGAATGGTGATTTTGCACTTAATACGCTTTACTATGCTG
AGTATGAGAATAATGGGCCTGGGTCAAACCAAGCCCAACGTGTTAAGTGGCCTGGAATTAAGAAACTATCGCCCAAGCAA
GCTCTTCGATTTACTCCTGCTAGGTTTTTACGTGGTAACTTGTGGATTCCACCAAATCGTGTGCCTTACATGGGGAATTT
TCAGTAGATTCCAATTGGTGAATTTTCCACTTTCTGTGTGCTCTTTAAAAAAAAAAATGAAGGTGAATAATTTATATGCG
TGTCTTGTCTTAAAGTCCTGACTTGCCGAA
>12646933_protein_ID_12646934
MGVDGELKKKKCIIAGVITALLVLMVVAVGITTSRNTSHSEKIVPVQIKTATTAVEAVCAPTDYKETCVNSLMKASPDST
QPLDLIKLGFNVTIRSIEDSIKKASVELTAKAANDKDTKGALELCEKLMNDATDDLKKCLDNFDGFSIPQIEDFVEDLRV
WLSGSIAYQQTCMDTFEETNSKLSQDMQKIFKTSRELTSNGLAMITNISNLLGEFNVTGVTGDLGKYARKLLSAEDGIPS
WVGPNTRRLMATKGGVKANVVVAHDGSGQYKTINEALNAVPKANQKPFVIYIKQGVYNEKVDVTKKNTHVTFIGDGPTKT
KITGSLNYYIGKVKTYLTATVAINGDNFTAKNIGFENTAGPEGHQAVALRVSADLAVFYNCQIDGYQDTLYVHSHRQFFR
DCTVSGTVDFIFGDGIVVLQNCNIVVRKPMKSQSCMITAQGRSDKRESTGLVLQNCHITGEPAYIPVKSINKAYLGRPWK
EFSRTIIMGTTIDDVIDPAGWLPWNGDFALNTLYYAEYENNGPGSNQAQRVKWPGIKKLSPKQALRFTPARFLRGNLWIP
PNRVPYMGNFQ*
>12656458_construct_ID_YP0107
ATGACGTCCGTTAACGTTAAGCTCCTTTACCGTTACGTCTTAACCAACTTTTTCAACCTCTGTTTGTTCCCGTTAACGGC
GTTCCTCGCCGGAAAAGCCTCTCGGCTTACCATAAACGATCTCCACAACTTCCTTTCCTATCTCCAACACAACCTTATAA
CAGTAACTTTACTCTTTGCTTTCACTGTTTTCGGTTTGGTTCTCTACATCGTAACCCGACCCAATCCGGTTTATCTCGTT
GACTACTCGTGTTACCTTCCACCACCGCATCTCAAAGTTAGTGTCTCTAAAGTCATGGATATTTTCTACCAAATAAGAAA
AGCTGATACTTCTTCACGGAACGTGGCATGTGATGATCCGTCCTCGCTCGATTTCCTGAGGAAGATTCAAGAGCGTTCAG
GTCTAGGTGATGAGACGTACAGTCCTGAGGGACTCATTCACGTACCACCGCGGAAGACTTTTGCAGCGTCACGTGAAGAG
ACAGAGAAGGTTATCATCGGTGCGCTCGAAAATCTATTCGAGAACACCAAAGTTAACCCTAGAGAGATTGGTATACTTGT
GGTGAACTCAAGCATGTTTAATCCAACTCCTTCGCTATCCGCTATGGTCGTTAATACTTTCAAGCTCCGAAGCAACATCA
AAAGCTTTAATCTAGGAGGAATGGGTTGTAGTGCTGGTGTTATTGCCATTGATTTGGCTAAAGACTTGTTGCATGTTCAT
AAAAACACTTATGCTCTTGTGGTGAGCACTGAGAACATCACACAAGGCATTTATGCTGGAGAAAATAGATCAATGATGGT
TAGCAATTGCTTGTTTCGTGTTGGTGGGGCCGCGATTTTGCTCTCTAACAAGTCGGGAGACCGGAGACGGTCCAAGTACA
AGCTAGTTCACACGGTCCGAACGCATACTGGAGCTGATGACAAGTCTTTTCGATGTGTGCAACAAGAAGACGATGAGAGC
GGCAAAATCGGAGTTTGTCTGTCAAAGGACATAACCAATGTTGCGGGGACAACACTTACGAAAAATATAGCAACATTGGG
TCCGTTGATTCTTCCTTTAAGCGAAAAGTTTCTTTTTTTCGCTACCTTCGTCGCCAAGAAACTTCTAAAGGATAAAATCA
AGCATTACTATGTTCCGGATTTCAAGCTTGCTGTTGACCATTTCTGTATTCATGCCGGAGGCAGAGCCGTGATCGATGAG
CTAGAGAAGAACTTAGGACTATCGCCGATCGATGTGGAGGCATCTAGATCAACGTTACATAGATTTGGGAATACTTCATC
TAGCTCAATTTGGTATGAATTAGCATACATAGAGGCAAAGGGAAGAATGAAGAAAGGGAATAAAGCTTGGCAGATTGCTT
TAGGATCAGGGTTTAAGTGTAATAGTGCGGTTTGGGTGGCTCTACGCAATGTCAAGGCATCGGCAAATAGTCCTTGGCAA
CATTGCATCGATAGATATCCGGTTAAAATTGATTCTGATTTGTCAAAGTCAAAGACTCATGTCCAAAACGGTCGGTCCTA
A
>12656458_protein_ID_12656459
MTSVNVKLLYRYVLTNFFNLCLFPLTAFLAGKASRLTINDLHNFLSYLQHNLITVTLLFAFTVFGLVLYIVTRPNPVYLV
DYSCYLPPPHLKVSVSKVMDIFYQIRKADTSSRNVACDDPSSLDFLRKIQERSGLGDETYSPEGLIHVPPRKTFAASREE
TEKVIIGALENLFENTKVNPREIGILVVNSSMFNPTPSLSAMVVNTFKLRSNIKSFNLGGMGCSAGVIAIDLAKDLLHVH
KNTYALVVSTENITQGIYAGENRSMMVSNCLFRVGGAAILLSNKSGDRRRSKYKLVHTVRTHTGADDKSFRCVQQEDDES
GKIGVCLSKDITNVAGTTLTKNIATLGPLILPLSEKFLFFATFVAKKLLKDKIKHYYVPDFKLAVDHFCIHAGGRAVIDE
LEKNLGLSPIDVEASRSTLHRFGNTSSSSIWYELAYIEAKGRMKKGNKAWQIALGSGFKCNSAVWVALRNVKASANSPWQ
HCIDRYPVKIDSDLSKSKTHVQNGRS *
>12660077_construct_ID_YP0049
TCTAGATGAATACTATACCGACGATGACTACACACACAAGGAAATATATATATCAGCTTTCTTTTCACCTAAAAGTGGTC
CCGGTTTAGAATCTAATTCCTTTATCTCTCATTTTCTTCTGCTTCACATTCCCGCTAGTCAAATGTTAATAAGTGCACAC
AACGTTTTCTCGAAGCATTAGAATGTCCTCCTCTTAATTAATCTCCTTCTGATTAGATTCTCAATAGAGTTTAAATTTGT
TAATGGAGAGATATATTGGGACCCTCAAGGCTTCTAATTATACCACGTTTGGCATAATTCTCTATCGTTTGGGGCCACAT
CTTTCACACTTCATTACCTTATCACCAAAACATAAAATCAATCAACTTTTTTTTGCCTTATTGATTGTGTTGGATCCCTC
CAAAATTAAAACTTGTGTTCCCCACAAAAGCTTACCCAATTTCACTTCAATCTTAACAAATAGGACCACCACTACCACGT
ACGGTTTGCATCATACAAACCACAAACTCCTTCTTCATTACAATTATTATATCATCTACTAAAACCTCTTTCTCCCTCTC
TCTTTCTTGTTCTTAGTGCTAAATTTTCTTTGTTCAGGAGAAATATAATGGACCTCAAGTATTCAGCATCTCATTGCAAC
TTATCCTCAGACATGAAGCTCAGGCGTTTTCATCAGCATCGAGGAAAAGGAAGAGAAGAAGAGTATGATGCTTCTTCTCT
CAGCTTGAACAATCTGTCAAAACTTATTCTTCCTCCACTTGGTGTTGCTAGCTATAACCAGAATCACATCAGGTCTAGTG
GATGGATCATCTCACCTATGGACTCAAGATACAGGTGCTGGGAATTTTATATGGTGCTTTTAGTGGCATACTCTGCGTGG
GTTTACCCTTTTGAAGTTGCATTTCTGAATTCATCACCAAAGAGAAACCTTTGTATCGCGGACAACATCGTAGACTTGTT
CTTCGCGGTTGACATTGTCTTGACGTTTTTCGTTGCTTACATAGACGAAAGAACACAGCTTCTTGTCCGTGAACCTAAAC
AGATTGCAGTGAGGTACCTATCAACATGGTTCTTGATGGATGTTGCATCAACTATACCATTTGACGCTATTGGATACTTA
ATCACTGGCACATCCACGTTAAATATCACTTGTAATCTCTTGGGATTACTTAGATTTTGGCGACTTCGAAGAGTTAAACA
CCTCTTCACTAGGCTCGAGAAGGACATAAGATATAGCTATTTCTGGATTCGCTGCTTTCGACTTCTATCAGTGACATTGT
TTCTAGTGCACTGTGCTGGATGCAGTTATTACCTAATAGCAGACAGATATCCACACCAAGGAAAGACATGGACTGATGCG
ATCCCTAATTTCACAGAGACAAGTCTTTCCATCAGATACATTGCAGCTATATATTGGTCTATCACTACAATGACCACAGT
GGGATATGGAGATCTTCATGCAAGCAACACTATTGAAATGGTATTCATAACAGTCTACATGTTATTCAATCTTGGCCTCA
CTGCTTACCTTATTGGTAACATGACTAATTTGGTCGTGGAAGGGACTCGTCGTACCATGGAATTTAGGAATAGCATTGAA
GCAGCGTCAAACTTTGTTAACAGAAACAGATTGCCTCCTAGATTAAAAGACCAGATATTAGCTTACATGTGTTTAAGGTT
TAAAGCAGAGAGCTTAAATCAGCAACATCTTATTGACCAGCTCCCAAAATCTATCTACAAAAGCATTTGTCAACATCTTT
TTCTTCCATCTGTTGAAAAAGTTTACCTCTTCAAAGGCGTCTCAAGAGAAATACTTCTTCTTCTGGTTTCAAAAATGAAG
GCTGAGTATATACCACCAAGAGAGGATGTCATTATGCAGAACGAAGCGCCGGATGATGTTTACATAATTGTGTCAGGAGA
AGTTGAGATCATTGATTCAGAGATGGAGAGAGAGTCTGTTTTAGGCACTCTACGTTGTGGAGACATATTTGGAGAAGTTG
GAGCACTTTGTTGCAGACCACAAAGCTACACTTTTCAAACTAAGTCTTTATCACAGCTTCTCCGACTCAAAACATCTTTC
CTTATTGAGACAATGCAGATTAAACAACAAGACAATGCCACAATGCTCAAGAACTTCTTGCAGCATCACAAAAAGCTGAG
TAATTTAGACATTGGTGATCTAAAGGCACAACAAAATGGCGAAAACACCGATGTTGTTCCTCCTAACATTGCCTCAAATC
TCATCGCTGTGGTGACTACAGGCAATGCAGCTCTTCTTGATGAGCTACTTAAGGCTAAGTTAAGCCCTGACATTACAGAT
TCCAAAGGAAAAACTCCATTGCATGTAGCAGCTTCTAGAGGATATGAAGATTGTGTTTTAGTACTCTTAAAGCACGGTTG
CAACATCCACATAAGAGATGTGAATGGTAATAGTGCTCTATGGGAAGCAATAATATCGAAGCATTACGAGATATTCAGAA
TCCTTTATCATTTCGCAGCCATATCGGATCCACACATAGCTGGAGATCTTCTATGTGAAGCAGCGAAACAGAACAATGTA
GAAGTCATGAAGGCTCTTTTAAAACAGGGGCTTAACGTCGACACAGAGGATCACCATGGCGTCACAGCTTTACAGGTCGC
TATGGCGGAGGATCAGATGGACATGGTGAATCTCCTGGCGACGAACGGTGCAGATGTAGTTTGTGTGAATACACATAATG
AATTCACACCATTGGAGAAGTTAAGAGTTGTGGAAGAAGAAGAAGAAGAAGAACGAGGAAGAGTGAGTATTTACAGAGGA
CATCCATTGGAGAGGAGAGAAAGAAGTTGCAATGAAGCTGGGAAGCTTATTCTTCTTCCTCCTTCACTTGATGACCTCAA
GAAAATTGCAGGAGAGAAGTTTGGGTTTGATGGAAGTGAGACGATGGTGACGAATGAAGATGGAGCTGAGATTGACAGTA
TTGAAGTGATTAGAGATAATGACAAACTCTACTTTGTCGTAAACAAGATAATTTAGAAGTTGAAAAATTATAACGAAATG
AAGTTTGAGATAAGAGAGAGCGTGACAAAAAAATGAAAAACAAATTGTAATATTTATATGCGTCCATCAAAGTGAGATGT
AACACATATTTGGGTAAGAAACGTTCCAAATCCCTGACGTAGCTCGAG
>12660077_protein_ID_12660078
MDLKYSASHCNLSSDMKLRRFHQHRGKGREEEYDASSLSLNNLSKLILPPLGVASYNQNHIRSSGWIISPMDSRYRCWEF
YMVLLVAYSAWVYPFEVAFLNSSPKRNLCIADNIVDLFFAVDIVLTFFVAYIDERTQLLVREPKQIAVRYLSTWFLMDVA
STIPFDAIGYLITGTSTLNITCNLLGLLRFWRLRRVKHLFTRLEKDIRYSYFWIRCFRLLSVTLFLVHCAGCSYYLIADR
YPHQGKTWTDAIPNFTETSLSIRYIAAIYWSITTMTTVGYGDLHASNTIEMVFITVYMLFNLGLTAYLIGNMTNLVVEGT
RRTMEFRNSIEAASNFVNRNRLPPRLKDQILAYMCLRFKAESLNQQHLIDQLPKSIYKSICQHLFLPSVEKVYLFKGVSR
EILLLLVSKMKAEYIPPREDVIMQNEAPDDVYIIVSGEVEIIDSEMERESVLGTLRCGDIFGEVGALCCRPQSYTFQTKS
LSQLLRLKTSFLIETMQIKQQDNATMLKNFLQHHKKLSNLDIGDLKAQQNGENTDVVPPNIASNLIAVVTTGNAALLDEL
LKAKLSPDITDSKGKTPLHVAASRGYEDCVLVLLKHGCNIHIRDVNGNSALWEAIISKHYEIFRILYHFAAISDPHIAGD
LLCEAAKQNNVEVMKALLKQGLNVDTEDHHGVTALQVAMAEDQMDMVNLLATNGADVVCVNTHNEFTPLEKLRVVEEEEE
EERGRVSIYRGHPLERRERSCNEAGKLILLPPSLDDLKKIAGEKFGFDGSETMVTNEDGAEIDSIEVIRDNDKLYFVVNK
II*
>12661844_construct_ID_YP0092
ATGGCCGAGGATTTGGACAAGCCATTGCTGGATCCTGATACTTTCAACAGAAAAGGAATTGATTTGGGTATATTGCCGTT
GGAGGAGGTTTTTGAATACCTAAGAACATCGCCTCAAGGGCTTTTATCTGGAGATGCTGAAGAGAGATTGAAGATATTTG
GTCCTAACAGACTTGAAGAGAAACAGGAGAACAGATTTGTGAAATTCTTAGGTTTTATGTGGAATCCCTTGTCATGGGTT
ATGGAAGCTGCTGCATTGATGGCCATTGCCCTCGCTAATAGTCAAAGTCTAGGTCCTGACTGGGAAGACTTTACTGGAAT
CGTTTGCCTTTTGCTGATCAACGCAACAATCAGCTTCTTTGAAGAAAACAATGCTGGGAATGCTGCTGCAGCTCTTATGG
CTCGCTTGGCTTTAAAAACAAGAGTTCTTAGAGATGGACAGTGGCAAGAACAAGATGCTTCTATCTTGGTACCTGGTGAT
ATAATTAGCATTAAGCTTGGGGATATCATTCCTGCAGATGCTCGCCTTCTTGAAGGAGACCCCTTGAAGATTGATCAGTC
AGTGCTGACCGGAGAATCACTACCTGTGACCAAGAAGAAGGGTGAACAGGTCTTTTCTGGCTCTACTTGTAAACAAGGTG
AAATAGAAGCTGTTGTGATAGCAACTGGATCGACCACCTTCTTTGGAAAAACAGCACGCTTGGTGGACAGTACAGATGTA
ACTGGACATTTTCAGCAGGTTCTTACATCGATTGGAAACTTCTGCATTTGCTCCATTGCTGTTGGAATGGTTCTTGAAAT
CATTATCATGTTCCCTGTACAACATCGCTCTTACAGAATTGGGATCAATAATCTTCTTGTACTACTGATTGGAGGGATAC
CCATTGCCATGCCCACTGTACTATCTGTAACGCTTGCCATTGGATCTCATCGACTTTCACAACAGGGTGCCATTACGAAA
AGAATGACCGCAATAGAGGAAATGGCTGGGATGGATGTACTCTGCTGTGATAAAACTGGAACCCTTACTTTGAACAGTCT
TACCGTTGATAAAAATCTTATTGAGGTATTCGTTGACTACATGGACAAGGATACAATTTTGTTGCTTGCAGGCCGAGCTT
CACGACTAGAAAATCAGGATGCTATAGATGCAGCCATTGTTAGCATGCTTGCAGATCCCAGAGAGGCACGTGCAAACATT
AGAGAAATCCATTTCTTACCATTCAATCCTGTGGACAAACGTACTGCAATAACGTATATTGATTCCGATGGAAAATGGTA
TCGTGCTACCAAAGGTGCTCCTGAACAGGTTCTAAACTTGTGTCAGCAGAAAAATGAGATTGCGCAAAGAGTTTATGCCA
TCATTGATAGATTTGCAGAAAAGGTTTGAGGTCTCTTGCGGTTGCTTATCAGGTTCCAGAGAAAAGCAAGCAACAACAGT
CCTGGAGGACCATGGAGGTTCTGTGGTCTGTTGCCACTGTTTGATCCCCCAAGGCATGATAGCGGTGAAACCATCCTTAG
AGCTCTTAGCCTGGGAGTTTGCGTTAAGATGATCACTGGTGATCAATTGGCGATTGCAAAGGAGACAGGCAGACGTCTTG
GAATGGGAACCAACATGTATCCTTCTTCCTCTTTGTTAGGCCACAACAATGATGAGCATGAAGCCATTCCAGTGGATGAG
CTAATTGAAATGGCAGATGGATTTGCTGGAGTTTTCCCTGAACATAAGTATGAGATTGTAAAGATTTTACAAGAAATGAA
GCATGTGGTTGGAATGACCGGAGATGGTGTGAATGATGCTCCTGCTCTCAAAAAAGCTGACATCGGAATAGCTGTCGCAG
ATGCAACAGATGCTGCAAGAAGTTCTGCTGACATAGTACTAACTGATCCCGGCTTAAGTGTAATTATCAGTGCTGTCTTG
ACCAGCAGAGCCATTTTCCAGCGGATGAGGAACTATACAGTATATGCAGTCTCTATCACCATACGCATACTTGGTTTTAC
ACTTTTAGCGTTGATATGGGAATACGACTTCCCACCTTTCATGGTTCTGATAATCGCAATACTCAATGACGGGACTATCA
TGACTATTTCTAAAGATCGAGTTAGGCCATCTCCTACACCCGAGAGTTGGAAGCTCAACCAGATATTTGCGACAGGAATT
GTCATTGGAACATATCTAGCATTGGTCACCGTCCTGTTTTACTGGATCATTGTTTCTACCACCTTCTTCGAGAAACACTT
CCATGTAAAATCAATTGCCAACAACAGTGAACAAGTGTCATCCGCGATGTATCTCCAAGTGAGCATCATCAGTCAGGCAC
TCATATTTGTAACACGTAGTCGAGGCTGGTCATTTTTTGAACGTCCCGGGACTCTCCTGATTTTTGCCTTCATTCTTGCT
CAACTTGCGGCTACATTAATTGCTGTGTATGCCAACATCAGCTTTGCTAAAATCACCGGCATTGGATGGAGATGGGCAGG
TGTTATATGGTTATACAGTCTGATATTTTACATACCTCTAGATGTTATAAAGTTTGTCTTTCACTACGCATTGAGTGGAG
AAGCTTGGAATCTCGTATTGGACCGTAAGACAGCTTTTACTTACAAGAAAGATTATGGGAAAGATGATGGATCGCCCAAT
GTAACCATCTCTCAGAGAAGTCGTTCCGCAGAAGAACTCAGAGGAAGCCGTTCTCGCGCTTCTTGGATCGCTGAACAAAC
CAGGAGGCGTGCAGAAATCGCCAGGCTTCTAGAGGTTCATTCAGTGTCAAGGCATTTAGAATCTGTGATCAAACTCAAAC
AAATTGACCAAAGGATGATCCGTGCAGCTCATACTGTCTAA
>12661844_protein_ID_12661845
MAEDLDKPLLDPDTFNRKGIDLGILPLEEVFEYLRTSPQGLLSGDAEERLKIFGPNRLEEKQENRFVKFLGFMWNPLSWV
MEAAALMAIALANSQSLGPDWEDFTGIVCLLLINATISFFEENNAGNAAAALMARLALKTRVLRDGQWQEQDASILVPGD
IISIKLGDIIPADARLLEGDPLKIDQSVLTGESLPVTKKKGEQVFSGSTCKQGEIEAVVIATGSTTFFGKTARLVDSTDV
TGHFQQVLTSIGNFCICSIAVGMVLEIIIMFPVQHRSYRIGINNLLVLLIGGIPIANPTVLSVTLAIGSHRLSQQGAITK
RMTAIEEMAGMDVLCCDKTGTLTLNSLTVDKNLIEVFVDYMDKDTILLLAGRASRLENQDAIDAAIVSMLADPREARANI
REIHFLPFNPVDKRTAITYIDSDGKWYRATKGAPEQVLNLCQQKNEIAQRVYAIIDRFAEKGLRSIAVAYQEIPEKSNNS
PGGPWRFCGLLPLFDPPRHDSGETILRALSLGVCVKMITGDQLAIAKETGRRLGMGTNMYPSSSLLGNNNDEHEAIPVDE
LIEMADGFAGVFPEHKYEIVKILQEMKHVVGMTGDGVNDAPALKKADIGIAVADATDAARSSADIVLTDPGLSVIISAVL
TSRAIFQRMRNYTVYAVSITIRILGFTLLALIWEYDFPPFMVLIIAILNDGTIMTISKDRVRPSPTPESWKLNQIFATGI
VIGTYLALVTVLFYWIIVSTTFFEKHFHVKSIANNSEQVSSAMYLQVSIISQALIFVTRSRGWSFFERPGTLLIFAFILA
QLAATLIAVYANISFAKITGIGWRWAGVIWLYSLIFYIPLDVIKFVFHYALSGEAWNLVLDRKTAFTYKKDYGKDDGSPN
VTISQRSRSAEELRGSRSRASWIAEQTRRRAEIARLLEVHSVSRHLESVIKLKQIDQRMIRAAHTV*
>12664333_construct_ID_YP0030
ATTCCAATCTCTCAAGAAAATCTACAGTTCCTCCAAATAATAATACCCTCCCTCTAAGGCAACTAATTTTCAGCAATCAT
GTCCGGGACTATTAATCCCCCGGACGGAGGAGGGTCCGGTGCAAGAAACCCACCAGTCGTTCGTCAGAGAGTGCTAGCTC
CTCCGAAAGCGGGTTTACTAAAGGACATCAAGTCCGTGGTTGAAGAAACTTTCTTCCATGATGCTCCGCTTAGGGATTTC
AAGGGCCAAACCCCAGCTAAAAAAGCGTTGCTCGGGATCCAGGCTGTCTTCCCGATCATCGGGTGGGCCAGAGAATACAC
TCTTCGCAAATTTAGAGGTGATCTCATCGCCGGTCTCACCATTGCTAGTCTTTGTATCCCTCAGGATATCGGATATGCAA
AACTCGCGAATGTCGATCCGAAATACGGACTTTATTCGAGTTTCGTGCCACCGCTGATTTACGCGGGCATGGGGAGTTCT
AGGGATATTGCGATAGGACCAGTCGCTGTGGTGTCTCTTCTTGTGGGAACTTTGTGCCAGGCCGTGATCGACCCAAAGAA
AAACCCGGAGGATTATCTCCGACTTGTCTTCACTGCCACTTTCTTTGCTGGCATTTTCCAAGCCGGCCTCGGATTTCTAC
GGTTGGGATTCTTGATAGACTTTCTGTCGCATGCGGCCGTGGTTGGGTTCATGGGAGGAGCAGCCATCACAATCGCTCTC
CAACAGCTTAAGGGCTTTCTTGGCATCAAAACATTTACCAAGAAAACTGATATTGTTTCTGTCATGCACTCCGTATTCAA
AAACGCTGAGCATGGGTGGAATTGGCAAACTATAGTCATTGGCGCCAGTTTCTTGACCTTTCTTCTCGTCACCAAATTCA
TTGGGAAGAGAAACAGGAAACTATTTTGGGTTCCGGCAATTGCGCCTCTTATTTCAGTCATTATCTCTACCTTCTTTGTC
TTCATTTTTCGTGCTGATAAACAAGGAGTCCAAATTGTGAAACATATAGATCAAGGAATCAATCCGATTTCCGTTCATAA
GATTTTCTTCTCCGGAAAATATTTCACCGAAGGAATCCGAATCGGAGGCATTGCGGGTATGGTCGCCTTAACGGAGGCTG
TAGCGATTGCAAGAACATTTGCGGCAATGAAAGACTATCAAATTGATGGAAACAAAGAGATGATTGCCCTAGGGACTATG
AACGTCGTCGGTTCAATGACCTCTTGTTACATTGCCACGGGTTCGTTTTCGCGATCTGCCGTGAACTTCATGGCGGGAGT
CGAAACGGCGGTTTCAAACATAGTTATGGCCATAGTTGTAGCTCTAACCTTAGAGTTCATCACACCACTCTTCAAGTACA
CTCCAAATGCTATCCTCGCGGCCATCATTATATCGGCTGTCCTCGGTCTTATCGATATTGACGCAGCGATTCTCATATGG
AGGATCGATAAACTCGACTTCTTGGCTTGCATGGGAGCTTTCTTAGGAGTCATCTTCATCTCGGTTGAGATCGGTCTCTT
GATCGCTGTGGTGATCTCTTTTGCAAAGATATTGCTTCAAGTGACGAGACCAGAACCACGGTTCTAGGGAAGCTCGCCAA
ATTCGAATGTATATCGGAACACTCTACAGTATCCGGACGCTGCCCAAATTCCCGGAATCTTGATCATCCGTGTTGACTCG
GCCATCTACTTTTCCAACTCCAACTATGTCCGAGAAAGGGCATCAAGATGGGTGCGAGAGGAGCAAGAAAATGCTAAGGA
ATATGGCATGCCGGCAATCAGATTTGTGATTATTGAGATGTCACCGGTTACCGATATCGATACCAGTGGTATCCACTCCA
TCGAAGAACTTCTCAAGAGCCTCGAGAAGCAAGAAATTCAGTTGATTCTAGCAAATCCAGGACCAGTGGTGATTGAGAAA
CTTTATGCTTCAAAGTTCGTCGAGGAGATTGGAGAGAAAAATATCTTCCTTACTGTTGGCGACGCGGTCGCAGTTTGTTC
TACGGAAGTGGCTGAGCAACAAACTTAATATCGTCTATTCATATACATAAACACATCCATATATGTATGTGTATATATAT
ATGAAAGAAACTAATTTAAGAACTATGGGTTATTTTCATTTTTTTGAGATGATATGATATTATGTGTGTAATATATGCAT
GATTGTTGAATTTGTTTGGTTCACACAATGGTGAGATGGGAACAAAGTCGAACGTTTGACTTTTATTTTTATTTTTTAAT
CTTTCAAATGTTATTTTCTCGTGATTTGTGTTTCGTTTGAGATGATGAATAAATTGTATTTTCAACTTATA
>12664333_protein_ID_12664334
MSGTINPPDGGGSGARNPPVVRQRVLAPPKAGLLKDIKSVVEETFFHDAPLRDFKGQTPAKKALLGIQAVFPIIGWAREY
TLRKFRGDLIAGLTIASLCIPQDIGYAKLANVDPKYGLYSSFVPPLIYAGMGSSRDIAIGPVAVVSLLVGTLCQAVIDPK
KNPEDYLRLVFTATFFAGIFQAGLGFLRLGFLIDFLSHAAVVGFMGGAAITIALQQLKGFLGIKTFTKKTDIVSVMHSVF
KNAEHGWNWQTIVIGASFLTFLLVTKFIGKRNRKLFWVPAIAPLISVIISTFFVFIFRADKQGVQIVKHIDQGINPISVH
KIFFSGKYFTEGIRIGGIAGMVALTEAVAIARTFAANKDYQIDGNKEMIALGTMNVVGSMTSCYIATGSFSRSAVNFMAG
VETAVSNIVMAIVVALTLEFITPLFKYTPNAILAAIIISAVLGLIDIDAAILIWRIDKLDFLACMGAFLGVIFISVEIGL
LIAVVISFAKILLQVTRPRTTVLGKLPNSNVYRNTLQYPDAAQIPGILIIRVDSAIYFSNSNYVRERASRWVREEQENAK
EYGMPAIRFVIIEMSPVTDIDTSGIHSIEELLKSLEKQEIQLILANPGPVVIEKLYASKFVEEIGEKNIFLTVGDAVAVC
STEVAEQQT*
>12669615_construct_ID_YP0204
AAACTCAGTCATTATATTTATTTTTGTTGTATTTCAACGTTCAATCTCTGAAAATGAAATATGCATTGATTCTTGTTCTC
TTTTTTGTTGTCTTCATATGGCAATCAAGCTCATCATCAGCAAACTCGGAGACTTTCACACAATGCCTAACCTCAAACTC
CGACCCCAAACATCCCATCTCCCCCGCTATCTTCTTCTCCGGAAATGGCTCCTACTCCTCCGTATTACAAGCCAACATCC
GTAACCTCCGCTTCAACACCACCTCAACTCCGAAACCCTTCCTCATAATCGCCGCAACACATGAATCCCATGTGCAAGCC
GCGATTACTTGCGGGAAACGCCACAACCTTCAGATGAAAATCAGAAGTGGAGGCCACGACTACGATGGCTTGTCATACGT
TACATACTCTGGCAAACCGTTCTTCGTCCTCGACATGTTTAACCTCCGTTCGGTGGATGTCGACGTGGCAAGTAAGACCG
CGTGGGTCCAAACCGGTGCCATACTCGGAGAAGTTTATTACTATATATGGGAGAAGAGCAAAACCCTAGCTTATCCCGCC
GGAATTTGTCCCACGGTTGGTGTCGGTGGCCATATCAGTGGTGGAGGTTACGGTAACATGATGAGAAAATACGGTCTCAC
CGTAGATAATACCATCGATGCAAGAATGGTCGACGTAAATGGAAAAATTTTGGATAGAAAATTGATGGGAGAAGATCTCT
ACTGGGCAATAAACGGAGGAGGAGGAGGGAGCTACGGCGTCGTATTGGCCTACAAAATAAACCTTGTTGAAGTCCCAGAA
AACGTCACCGTTTTCAGAATCTCCCGGACGTTAGAACAAAATGCGACGGATATCATTCACCGGTGGCAACAAGTTGCACC
GAAGCTTCCCGACGAGCTTTTCATAAGAACAGTCATTGACGTAGTAAACGGCACTGTTTCATCTCAAAAGACCGTCAGGA
CAACATTCATAGCAATGTTTCTAGGAGACACGACAACTCTACTGTCGATATTAAACCGGAGATTCCCAGAATTGGGTTTG
GTCCGGTCTGACTGTACCGAAACAAGCTGGATCCAATCTGTGCTATTCTGGACAAATATCCAAGTTGGTTCGTCGGAGAC
ACTTCTACTCCAAAGGAATCAACCCGTGAACTACCTCAAGAGGAAATCAGATTACGTACGTGAACCGATTTCAAGAACCG
GTTTAGAGTCAATTTGGAAGAAAATGATCGAGCTTGAAATTCCGACAATGGCTTTCAATCCATACGGTGGTGAGATGGGG
AGGATATCATCTACGGTGACTCCGTTCCCATACAGAGCCGGTAATCTCTGGAAGATTCAGTACGGTGCGAATTGGAGAGA
TGAGACTTTAACCGACCGGTACATGGAATTGACGAGGAAGTTGTACCAATTCATGACACCATTTGTTTCCAAGAATCCGA
GACAATCGTTTTTCAATTACCGTGATGTTGATTTGGGTATTAATTCTCATAATGGTAAAATCAGTAGTTATGTGGAAGGT
AAACGTTACGGGAAGAAGTATTTCGCAGGTAATTTCGAGAGATTGGTGAAGATTAAGACGAGAGTTGATAGTGGTAATTT
CTTTAGGAACGAACAGAGTATTCCTGTGTTACCATAAGTGTATTTATTTGATTATTGGTTAGTGAAATTTGTTGTTGTAT
AATGATTATATGTCGTATTTTTATTTATTATTAGTAATTTATAAAGTTTGATATT
>12669615_protein_ID_12669617
MKYALILVLFFVVFIWQSSSSSANSETFTQCLTSNSDPKHPISPAIFFSGNGSYSSVLQANIRNLRFNTTSTPKPFLIIA
ATHESHVQAAITCGKRHNLQMKIRSGGHDYDGLSYVTYSGKPFFVLDMFNLRSVDVDVASKTAWVQTGAILGEVYYYIWE
KSKTLAYPAGICPTVGVGGHISGGGYGNMMRKYGLTVDNTIDARMVDVNGKILDRKLMGEDLYWAINGGGGGSYGVVLAY
KINLVEVPENVTVFRISRTLEQNATDIIHRWQQVAPKLPDELFIRTVIDVVNGTVSSQKTVRTTFIAMFLGDTTTLLSIL
NRRFPELGLVRSDCTETSWIQSVLFWTNIQVGSSETLLLQRNQPVNYLKRKSDYVREPISRTGLESIWKKMIELEIPTMA
FNPYGGEMGRISSTVTPFPYRAGNLWKIQYGANWRDETLTDRYMELTRKLYQFMTPFVSKNPRQSFFNYRDVDLGINSHN
GKISSYVEGKRYGKKYFAGNFERLVKIKTRVDSGNFFRNEQSIPVLP*
>12670159_construct_ID_YP0040
AGCATCCACACACACTTTGAATGCTCAATCAAAGCTTCTTCATAGTTAAACTTCCACACAACGTCAAAACTCGAGAAGAA
GATGAAAGAGAGAGATTCAGAGAGTTTTGAATCTCTCTCACATCAAGTTCTCCCAAACACTTCAAATTCAACACACATGA
TCCAGATGGCCATGGCCAACTCAGGTTCATCTGCAGCCGCACAAGCCGGTCAAGACCAGCCTGACCGGTCAAAGTGGCTG
CTTGACTGTCCTGAACCACCTAGCCCGTGGCATGAGCTCAAAAGACAAGTCAAAGGCTCTTTCCTAACCAAAGCCAAAAA
GTTCAAGTCACTTCAAAAACAGCCTTTCCCAAAACAAATCCTCTCTGTCCTCCAAGCCATTTTCCCAATCTTCGGTTGGT
GCAGAAACTATAAACTCACCATGTTCAAGAACGATCTCATGGCTGGTTTAACCCTCGCTAGCCTCTGCATTCCGCAGAGC
ATTGGTTATGCAACTCTTGCAAAGCTTGATCCTCAATATGGCCTATATACGAGTGTGGTACCACCATTGATATATGCATT
GATGGGGACATCAAGAGAGATAGCAATCGGACCGGTGGCTGTAGTATCTCTTCTTATATCTTCAATGTTGCAGAAACTCA
TCGATCCAGAAACAGATCCCTTGGGATACAAGAAACTGGTCCTAACCACAACCTTCTTCGCCGGGATCTTCCAAGCTTCT
TTCGGTTTATTCAGGTTAGGGTTTCTGGTGGATTTTCTGTCGCACGCAGCCATAGTTGGGTTCATGGGTGGTGCAGCCAT
TGTAATTGGACTCCAACAGCTTAAAGGTTTGCTTGGTATCACTAACTTCACCACCAACACTGACATTGTCTCTGTTCTTC
GAGCTGTCTGGAGATCTTGTCAACAACAATGGAGCCCTCACACTTTCATCCTCGGATGTTCTTTCCTCAGTTTTATCCTT
ATTACTCGCTTCATCGGGAAGAAGTATAAGAAGCTGTTTTGGCTACCGGCAATAGCTCCGTTGATCGCCGTGGTAGTGTC
AACACTAATGGTGTTTCTGACTAAAGCCGACGAGCATGGTGTGAAGACAGTGAGGCACATCAAAGGAGGTCTTAATCCAA
TGTCCATTCAGGATCTCGACTTTAATACTCCTCATCTCGGACAAATCGCTAAAATCGGATTAATCATTGCCATTGTTGCT
CTAACCGAGGCGATTGCGGTGGGGAGGTCGTTCGCCGGAATAAAAGGGTACAGACTCGATGGAAACAAAGAAATGGTGGC
CATTGGATTTATGAATGTTCTCGGTTCCTTCACATCTTGTTACGCTGCTACTGGTTCATTCTCTCGGACGGCCGTGAATT
TTGCGGCAGGATGTGAGACAGCAATGTCCAACATTGTTATGGCGGTTACGGTGTTTGTAGCACTCGAGTGTCTAACGAGG
CTTCTCTACTATACTCCAATCGCCATCCTCGCTTCAATAATTCTCTCAGCACTTCCGGGACTAATCAACATTAACGAGGC
TATTCACATTTGGAAAGTCGATAAATTCGATTTTCTTGCTCTCATTGGAGCTTTCTTTGGTGTTTTGTTCGCTTCCGTTG
AGATCGGACTTCTTGTCGCGGTGGTTATTTCGTTTGCCAAGATCATACTCATATCAATTCGTCCAGGGATAGAAACGCTT
GGAAGAATGCCCGGGACCGATACTTTTACAGATACTAATCAATATCCTATGACGGTTAAGACTCCCGGAGTGTTGATTTT
TCGTGTCAAGTCTGCATTGTTGTGCTTTGCCAATGCCAGTTCAATTGAGGAAAGGATTATGGGATGGGTCGATGAGGAAG
AAGAAGAAGAAAACACAAAGAGCAATGCCAAGAGAAAGATCCTCTTTGTAGTCCTTGATATGTCAAGTTTGATCAACGTC
GATACATCGGGGATTACTGCTTTGCTGGAACTGCATAACAAATTAATCAAAACTGGTGTTGAGCTAGTGATCGTTAACCC
GAAATGGCAAGTAATCCACAAGCTGAATCAAGCAAAGTTCGTCGACAGAATCGGTGGCAAAGTTTACTTGACGATCGGCG
AAGCTCTTGATGCTTGCTTTGGATTAAAAGTTTAAGAAACAGTTTTCAAAGGACCAGTTGTGTTACGGGTTATTGCATGT
GATGAATTTATGTGAGTTGTTGTGATTTAAATAATGTGATGCGTGCATGATCATGATTAATATTTAAGTACGTATGTGTA
ATAGAGTGCTTGGTCGTGACTGAATAAAGTCATGCAAACTATAATGTGAGGATCGATGGGTGTGTTTGTAACTCGATAGA
TTTGGAAATAATGTATAATATATGTAAGTTTGAGAATTATTGGTGTTTTGTATGATTGTTGAAATGTTATATAGAATCAG
GGATATATTTTTTGGGG
>12670159_protein_ID_12670160
MKERDSESFESLSHQVLPNTSNSTHMIQMAMANSGSSAAAQAGQDQPDRSKWLLDCPEPPSPWHELKRQVKGSFLTKAKK
FKSLQKQPFPKQILSVLQAIFPIFGWCRNYKLTMFKNDLMAGLTLASLCIPQSIGYATLAKLDPQYGLYTSVVPPLIYAL
MGTSREIAIGPVAVVSLLISSMLQKLIDPETDPLGYKKLVLTTTFFAGIFQASFGLFRLGFLVDFLSHAAIVGFMGGAAI
VIGLQQLKGLLGITNFTTNTDIVSVLRAVWRSCQQQWSPHTFILGCSFLSFILITRFIGKKYKKLFWLPAIAPLIAVVVS
TLMVFLTKADEHGVKTVRHIKGGLNPMSIQDLDFNTPHLGQIAKIGLIIAIVALTEAIAVGRSFAGIKGYRLDGNKEMVA
IGFMNVLGSFTSCYAATGSFSRTAVNFAAGCETAMSNIVMAVTVFVALECLTRLLYYTPIAILASIILSALPGLININEA
IHIWKVDKFDFLALIGAFFGVLFASVEIGLLVAVVISFAKIILISIRPGIETLGRMPGTDTFTDTNQYPMTVKTPGVLIF
RVKSALLCFANASSIEERIMGWVDEEEEEENTKSNAKRKILFVVLDMSSLINVDTSGITALLELHNKLIKTGVELVIVNP
KWQVIHKLNQAKFVDRIGGKVYLTIGEALDACFGLKV*
>12678173_construct_ID_YP0068
GAAATCCCTAAAATAGGAGGGAAJAATATATTGATCGTAGCTAGGGTTATCGACTCTTTTGTCAACCTCTCCATGGACTTT
TTCGGTTTTAACAGACCTCAGGTCTGCAAAGAACACAAAGTGCTGAACCTGTTTGCTGATAATCCTGAGATGAAAGCCTT
TTTCGAGAAGATATTTTATAGTTGGTATATCGACGTTGAAGGATTCGACACTTCGCTTCCTGAGGATGAGATGAAGGAGG
CCTTGACTAATCATTTCAAGTCATGTGGAGTAATCGCTATGGTTTCTTTCCGGAGACACCCTGAAACCGATGTTGTCAAC
GGCCTTGCTACTATTACCATGATGGGAAATGACGCTGATGAGAAGGTGATGCTACTTAATGGAAGTGAATTGGGAGGAAG
GAAACTTGTTGTCAAGGCCAACCCTACTCCCAGACTGAAACTTGACCATCTTAACCTTCCCTTTGGCGGCTCCTCTGTCC
CAGGTACATCATAAGTTTGGAGTCTCTTTGGTGTTTTCAGATCCAGATACAATGCAACCTGCTTTCTTTTCATCACTCGT
TGGGTCCTTATGAACTGTGAGACAATGAAACCCCCTTTGGGTCTTTCTTTCTTTGCCATGTTTAAATGTAAGCTCCATAT
GTATGACGTTTGTGTGTGGATGATTAAAGTAAGCTCTATTATCATTATCTAGTTTG
>12678173_protein_ID_12678174
MDFFGFNRPQVCKEHKVLNLFADNPEMKAFFEKIFYSWYIDVEGFDTSLPEDEMKEALTNHFKSCGVIANVSFRRHPETD
VVNGLATITMNGNDADEKVMLLNGSELGGRKLVVKANPTPRLKLDHLNLPFGGSSVPGTS*
>12679922_construct_ID_G0013
ATCTAATATCTCTTTCTCAATTTCGGTTCCACTTTCCTTTCGTTTGCAAAAACCCATCCCATCAAAAATAAACAAGAGGG
CCTAAAGAAGAATCCTAAAGACTTTACGGGTCTTGTTTAGGATAAAAGAAATGCCTGCCGGTGGATTCGTCGTCGGGGAT
GGCCAAAAGGCTTATCCCGGCAAACTCACTCCCTTTGTTCTCTTCACTTGCGTTGTTGCTGCCATGGGCGGTCTCATCTT
CGGATACGATATCGGAATCTCCGGTGGTGTGACGTCTATGCCGTCTTTCCTCAAGCGATTCTTCCCGTCGGTGTATCGGA
AACAACAAGAGGACGCGTCAACGAACCAGTACTGTCAGTACGATAGCCCGACGCTAACGATGTTCACATCGTCTCTATAT
CTAGCGGCGCTAATTTCGTCGCTGGTGGCTTCCACCGTGACAAGAAAGTTCGGACGGCGGCTCTCGATGCTCTTCGGCGG
CATACTCTTCTGCGCCGGAGCTCTCATCAATGGTTTCGCCAAACATGTTTGGATGCTCATCGTCGGTCGTATCTTGCTTG
GTTTCGGTATCGGTTTCGCTAATCAGGCTGTGCCACTGTACCTCTCTGAGATGGCTCCATACAAATACAGAGGAGCTTTA
AACATTGGTTTCCAGCTCTCAATTACAATCGGAATCCTCGTCGCCGAAGTGCTAAACTACTTCTTCGCCAAGATCAAAGG
CGGTTGGGGATGGCGGCTCAGTCTCGGAGGCGCGGTGGTTCCTGCCTTGATCATAACCATCGGCTCCCTCGTCCTCCCTG
ACACTCCCAATTCAATGATCGAGCGTGGCCAACACGAAGAAGCCAAAACCAAGCTCAGACGAATCCGTGGTGTCGATGAC
GTCAGCCAAGAGTTTGACGATTTGGTCGCCGCTAGTAAAGAGTCGCAGTCGATAGAGCACCCGTGGAGAAACCTCCTCCG
CCGCAAGTACCGACCACATCTCACAATGGCCGTTATGATTCCGTTCTTTCAACAGCTAACCGGAATCAATGTGATTATGT
TTTACGCTCCGGTTTTGTTCAACACCATTGGTTTCACGACCGATGCTTCTCTCATGTCCGCTGTGGTCACTGGCTCGGTT
AACGTGGCCGCTACGCTTGTTTCTATCTACGGTGTTGACAGATGGGGACGTCGGTTTCTCTTTCTTGAAGGTGGTACACA
AATGCTTATATGCCAGGCTGTGGTTGCAGCTTGCATAGGGGCCAAGTTTGGGGTAGACGGGACCCCTGGTGAGCTACCAA
AGTGGTATGCTATAGTGGTTGTAACGTTCATTTGCATCTATGTGGCGGGTTTTGCGTGGTCGTGGGGCCCACTAGGGTGG
TTAGTACCGAGTGAAATCTTCCCGTTGGAGATAAGGTCGGCGGCGCAGAGTATCACCGTGTCCGTGAACATGATCTTCAC
GTTCATTATCGCGCAAATCTTCTTGACGATGCTTTGTCATTTGAAGTTTGGGTTATTCCTTGTTTTCGCCTTTTTCGTGG
TGGTGATGTCGATCTTTGTATACATTTTCTTGCCGGAGACGAAAGGGATTCCGATAGAGGAGATGGGTCAAGTGTGGAGG
TCACACTGGTATTGGTCAAGGTTTGTGGAGGATGGTGAGTATGGGAATGCGCTTGAGATGGGCAAGAACAGTAACCAAGC
TGGAACGAAGCATGTTTGATTTATCATTGTTTTTAATGAGAGTTTTAAGAAAGAAAGAAAAAAGATTTGTAATTTCTAAT
GTCGTAAAGGAAAAAGTGTATTAGCCTAGATATTTATTGGTGTTTATATAATTCAATACCACATGAAGAAATTATGCATA
TGATTCTTCGTTAATTGTCTGTAATTGTTATACTCTTTACTTAAACCAAGTGTTTTCTCTTTG
>12679922_protein_ID_12679923
MPAGGFVVGDGQKAYPGKLTPFVLFTCVVAAMGGLIFGYDIGISGGVTSMPSFLKRFFPSVYRKQQEDASTNQYCQYDSP
TLTMFTSSLYLAALISSLVASTVTRKFGRRLSMLFGGILFCAGALINGFAKHVWMLIVGRILLGFGIGFANQAVPLYLSE
MAPYKYRGALNIGFQLSITIGILVAEVLNYFFAKIKGGWGWRLSLGGAVVPALIITIGSLVLPDTPNSMIERGQHEEAKT
KLRRIRGVDDVSQEFDDLVAASKESQSIEHPWRNLLRRKYRPHLTMAVMIPFFQQLTGINVIMFYAPVLFNTIGFTTDAS
LMSAVVTGSVNVAATLVSIYGVDRWGRRFLFLEGGTQMLICQAVVAACIGAKFGVDGTPGELPKWYAIVVVTFICIYVAG
FAWSWGPLGWLVPSEIFPLEIRSAAQSITVSVNMIFTFIIAQIFLTMLCHLKFGLFLVFAFFVVVMSIFVYIFLPETKGI
PIEEMGQVWRSHWYWSRFVEDGEYGNALEMGKNSNQAGTKHV*
>12688453_construct_ID_YP0192
TCATATTCACCTAAAAATCAGGTCCCCTCTCTTTATATCTCTAACATTCTTATATCAGATCATATTTTTTGGATTTCTTG
TTAAGTAACACCAATCTTTTAAAAGTGTTTTCAGGTTAATATAAAAGAATAATGATGTTTTCGGTGACGGTTGCGATCCT
TGTTTGTCTTATTGGCTACATTTACCGATCATTTAAGCCTCCACCACCGCGAATCTGCGGCCATCCTAACGGTCCTCCGG
TTACTTCTCCGAGAATCAAGCTCAGTGATGGAAGATATCTTGCTTATAGAGAATCTGGGGTTGATAGAGACAATGCTAAC
TACAAGATCATTGTCGTTCATGGCTTCAACAGCTCCAAAGACACTGAATTTCCCATCCCTAAGGATGTAATTGAGGAGCT
TGGGATATACTTTGTGTTCTACGATAGAGCAGGATATGGAGAAAGTGATCCACACCCATCACGCACTGTTAAGAGTGAAG
CATACGACATTCAAGAACTCGCCGATAAACTCAAGATCGGACCAAAGTTCTATGTTCTTGGTATATCACTAGGTGCTTAC
TCGGTTTATAGTTGCCTCAAATACATTCCCCACAGACTAGCTGGAGCAGTCTTAATGGTTCCATTTGTGAACTATTGGTG
GACTAAAGTGCCTCAAGAAAAATTGAGTAAAGCGTTGGAGCTAATGCCAAAGAAAGACCAATGGACGTTTAAAGTGGCTC
ATTATGTTCCGTGGTTGTTATATTGGTGGTTGACCCAAAAACTATTTCCGTCTTCGAGTATGGTCACGGGGAACAATGCG
TTATGCAGCGACAAAGATTTGGTCGTCATAAAGAAGAAAATGGAGAATCCACGCCCTGGCTTGGAAAAAGTTAGACAACA
AGGAGACCATGAATGTCTTCACCGGGACATGATAGCCGGATTCGCGACATGGGAATTCGACCCGACTGAATTAGAAAATC
CGTTTGCGGAAGGCGAAGGATCGGTCCACGTTTGGCAAGGGATGGAAGACAGAATCATTCCATACGAAATTAATCGATAT
ATATCAGAGAAGCTTCCATGGATTAAGTACCATGAGGTCTTAGGTTATGGACATCTTCTAAACGCCGAGGAGGAGAAATG
CAAAGACATTATCAAGGCACTTCTTGTCAACTGATGATCATCTCTACACAAGATGCCACGAAAAATATAGCATATTTAAT
AGATTTTATTTATGGATTATAATATTATAGCATATTATAAGTTTGTAAGTAAGATGAAAACCACTTGAAAGTC
>12688453_protein_ID_12688454
MMFSVTVAILVCLIGYIYRSFKPPPPRICGHPNGPPVTSPRIKLSDGRYLAYRESGVDRDNANYKIIVVHGFNSSKDTEF
PIPKDVIEELGIYFVFYDRAGYGESDPHPSRTVKSEAYDIQELADKLKIGPKFYVLGISLGAYSVYSCLKYIPHRLAGAV
LMVPFVNYWWTKVPQEKLSKALELMPKKDQWTFKVAHYVPWLLYWWLTQKLFPSSSMVTGNNALCSDKDLVVIKKKNENP
RPGLEKVRQQGDHECLHRDMIAGFATWEFDPTELENPFAEGEGSVHVWQGMEDRIIPYEINRYISEKLPWIKYHEVLGYG
HLLNAEEEKCKDIIKALLVN*
>12692181_construct_ID_YP0097
CATATCCAACAACAAAAACATAAGCTAAGAAAACGAAACTCAACTAATTTTGTTATCACCCAAAAAGAAGTTCAAACACA
ATGGCTTTCGCTTTGAGGTTCTTCACATGCCTTGTTTTAACGGTGTGCATAGTTGCATCAGTCGATGCTGCAATCTCATG
TGGCACAGTGGCAGGTAGCTTGGCTCCATGTGCAACCTATCTATCAAAAGGTGGGTTGGTGCCACCTTCATGTTGTGCAG
GAGTCAAAACTTTGAACAGTATGGCTAAAACCACACCAGACCGCCAACAAGCTTGCAGATGCATCCAGTCCACTGCGAAG
AGCATTTCTGGTCTCAACCCAAGTCTAGCCTCTGGCCTTCCTGGAAAGTGCGGTGTTAGCATTCCATATCCAATCTCCAT
GAGCACTAACTGCAACAACATCAAGTGAAATGGAAGCTTACGTCGTCGTTTTGGCGTTAAGAGTATGGTTTACCAGAAGT
ACTAGAATAAAATACGGCTATATATCTTAGCTGATATTACCATGTATTTGTTTTTGTCTCAATGCTTTGTCTTATTTTCA
TATCATATGTTGTATTGATGTGCTAAAACTATGATAATAGTACCTTATTAGTCATCTTC
>12703041_construct_ID_YP0007
ACAGAGACAACAAACTAAAGTTGGTGGTGATAGAGTGAGAGAGAAACATGGAAGGCAAAGAAGAAGACGTCAATGTTGGA
GCCAACAAGTTCCCAGAGAGACAGCCGATCGGTACGGCGGCTCAGACGGAGAGCAAGGACTATAAGGAACCACCACCGGC
GCCGTTTTTCGAACCCGGCGAGCTCAAATCTTGGTCTTTCTACAGAGCAGGGATAGCTGAGTTCATAGCCACTTTCCTTT
TCCTCTACGTCACCGTTTTGACAGTCATGGGTGTTAAGAGAGCTCCCAATATGTGTGCCTCTGTTGGAATCCAAGGCATC
GCTTGGGCTTTTGGTGGCATGATCTTTGCTCTTGTTTACTGTACTGCTGGAATCTCAGGAGGACATATTAATCCGGCGGT
GACTTTTGGTTTGTTCTTGGCGAGGAAGCTATCTTTAACCAGAGCTCTGTTCTACATAGTAATGCAGTGCCTTGGAGCTA
TATGTGGTGCTGGTGTGGTTAAAGGGTTTCAACCAGGGCTGTACCAGACGAATGGCGGTGGAGCTAATGTGGTGGCTCAT
GGTTACACAAAGGGTTCAGGTCTTGGTGCAGAGATTGTTGGAACTTTTGTTCTGGTTTACACTGTTTTCTCAGCTACTGA
TGCTAAGAGAAGTGCCAGAGACTCTCATGTCCCTATCTTGGCTCCGCTTCCPTTTGGGTTTGCTGTCTTCTTGGTGCACT
TGGCTACCATCCCAATTACTGGAACTGGCATTAACCCGGCCAGGAGTCTCGGAGCTGCCATCATCTACAACAAGGATCAT
GCTTGGGATGACCATTGGATCTTCTGGGTCGGTCCATTCATTGGTGCTGCGCTTGCTGCTCTGTACCATCAGATAGTCAT
TTTAATTCTATATGCTTTCTTCTTGTTTCCTATGTCATGTGTGATGATCTCTATATGTACCACTAGAGCTTTGATCTTGT
AACAGTGTAAATGTGTAATCTATTATGTATCAATGGCATTGTATCTTGTAACATTAATTATGTCAATGGAAGAATACATT
GTG
>12703041_protein_ID_12703042
MEGKEEDVNVGANKFPERQPIGTAAQTESKDYKEPPPAPFFEPGELKSWSFYRAGIAEFIATFLFLYVTVLTVMGVKRAP
NMCASVGIQGIAWAFGGMIFALVYCTAGISGGHINPAVTFGLFLARKLSLTRALFYIVMQCLGAICGAGVVKGFQPGLYQ
TNGGGANVVAHGYTKGSGLGAEIVGTFVLVYTVFSATDAKRSARDSHVPILAPLPIGFAVFLVHLATIPITGTGINPARS
LGAAIIYNKDHAWDDHWIFWVGPFIGAALAALYHQIVIRAIPFKSKT*
>12711515_construct_ID_YP0022
ATCTCACACCAAAACACAAAGCTCTCATCTTCTTTTAGTTTCCAAACTCACCCCCACAACTTTCATTTCTATCAACCAAA
CCCAAATGGGTCCAAGTTCGAGCCTCACCACCATCGTGGCGACTGTTCTTCTTGTGACATTGTTCGGTTCGGCCTACGCA
AGCAACTTCTTCGACGAGTTTGACCTCACTTGGGGTGACCACAGAGGCAAAATCTTCAACGGAGGAAATATGCTGTCTTT
GTCGCTGGACCAGGTTTCCGGGTCAGGTTTCAAATCCAAAAAAGAGTATTTGGTCGGTCGGATCGATATGCAGCTCAAAC
TTGTCGCCGGAAACTCGGCCGGCACCGTCACTGCTTACTACTTGTCTTCACAAGGAGCAACACATGACGAGATAGACTTT
GAGTTTCTAGGTAACGAGACAGGGAAGCCTTATGTTCTTCACACCAATGTCTTTGCTCAAGGGAAAGGAGACAGAGAGCA
ACAGTTTTATCTCTGGTTCGACCCAACCAAGAACTTCCACACTTACTCCATTGTCTGGAGACCCCAACACATCATATTCT
TGGTGGACAATTTACCCATTAGAGTGTTCAACAATGCAGAGAAGCTCGGCGTTCCTTTCCCAAAGAGTCAACCCATGAGG
ATCTACTCTAGCCTGTGGAATGCAGACGATTGGGCCACGAGAGGTGGTCTAGTCAAGACTGACTGGTCCAAGGCTCCTTT
CACAGCTTACTACAGAGGATTCAACGCTGCGGCTTGCACAGCCTCTTCAGGATGTGACCCTAAATTCAAGAGTTCTTTTG
GTGATGGTAAATTGCAAGTGGCAACCGAGCTCAATGCTTATGGCAGGAGGAGACTCAGATGGGTTCAGAAATACTTCATG
ATCTATAATTATTGCTCTGATCTCAAAAGGTTCCCTCGTGGATTCCCTCCAGAATGCAAGAAGTCCAGAGTCTGATGAAC
ACATATTACCTCATATTTCTCTGCTTGTTTGATGCAATTCTTAAATTCCTCTGTTATTCCATTGTACATTGTCAAGATCA
ATAAAGCATTCCTGGTTTCAAAAT
>12711515_protein_ID_12711517
MGPSSSLTTIVATVLLVTLFGSAYASNFFDEFDLTWGDHRGKIFNGGNMLSLSLDQVSGSGFKSKKEYLVGRIDMQLKLV
AGNSAGTVTAYYLSSQGATHDEIDFEFLGNETGKPYVLHTNVFAQGKGDREQQFYLWFDPTKNFHTYSIVWRPQHIIFLV
DNLPIRVFNNAEKLGVPFPKSQPMRIYSSLWNADDWATRGGLVKTDWSKAPFTAYYRGFNAAACTASSGCDPKFKSSFGD
GKLQVATELNAYGRRRLRWVQKYFMIYNYCSDLKRFPRGFPPECKKSRV*
>12713856_construct_ID_YP0126
AAGTTTCTCACATTTTCCAATAAAGCATCTAACTTACAATTAAAGACAATCCATGGCGATCAGAATCCCTCGTGTGCTGC
AATCATCGAAGCAGATTCTCCGACAAGCCAAACTGTTGTCATCATCTTCTTCTTCTAGCTCTCTTGATGTTCCCAAAGGC
TACTTAGCGGTTTACGTAGGAGAACAAAATATGAAGAGATTTGTAGTTCCGGTTTCGTACTTGGACCAGCCTTCATTTCA
AGATCTATTAAGAAAGGCAGAGGAAGAGTTTGGATTTGATCATCCAATGGGTGGCCTCACAATCCCTTGCAGTGAAGAAA
TTTTTATTGATCTTGCTTCTCGCTTCAACTGATCATGACTCACTCGATAACCTTACTTTTGTCATTGATTTTTGTACATT
TTGTTTTCCCAATTAGTTTTCTTCAAGAGATGAGATGACTTAGAAACAGCATCTCTCCTTGAAAGTGAAACAGAGACTTG
TAACACTCTTTTTCCTCACTTACAGTGAGTTGGACTCAAATCTAATCAAAACCATCATTTAGTCATC
>12713856_protein_ID_12713857
MAIRIPRVLQSSKQILRQAKLLSSSSSSSSLDVPKGYLAVYVGEQNMKRFVVPVSYLDQPSFQDLLRKAEEEFGFDHPMG
GLTIPCSEEIFIDLASRFN*
>12736079_construct_ID_YP0001
ATGAAAACACAATCAGCTTCACCGTTCTTCTTCGTCTCCTTCTTCTTCTTCTTCTTCTTCTTCTCTTCTCTGTTTCTTCT
CTCCTCTGCTTTAAACTCTGATGGAGTTCTCTTACTGAGTTTCAAATACTCTGTTCTTCTTGATCCTCTCTCTTTATTAC
AATCATGGAACTACGACCACGACAATCCTTGTTCATGGCGAGGTGTGTTGTGTAATAACGATTCAAGAGTTGTTACTTTA
TCTCTCCCAAACTCTAACCTCGTTGGTTCGATTCCTTCCGATCTGGGTTTCCTCCAAAACCTCCAAAGTCTTAATCTTTC
CAATAATTCACTCAATGGGTCATTACCGGTTGAGTTTTTCGCCGCCGATAAGCTCCGGTTTCTTGATTTATCAAATAACT
TGATCTCCGGCGAGATCCCTGTATCAATCGGAGGTTTACACAACCTCCAGACGTTAAATCTCTCCGATAACATCTTCACC
GGGAAACTACCAGCTAACTTAGCGTCTCTTGGAAGCTTAACGGAGGTTTCTCTGAAGAACAACTACTTCTCCGGCGAGTT
TCCCGGCGGCGGATGGAGATCGGTTCAGTATCTAGACATTTCTTCAAATCTAATCAACGGTTCACTCCCACCTGATTTCT
CCGGCGACAATCTCCGATACCTGAATGTCTCGTATAACCAAATCTCCGGAGAGATTCCTCCGAATGTTGGTGCCGGTTTT
CCTCAAAACGCCACCGTTGATTTCTCCTTCAACAATTTAACCGGTTCAATCCCAGATTCTCCGGTTTACCTTAACCAGAA
ATCAATTTCGTTTTCCGGAAACCCGGGTTTATGCGGAGGTCCGACCCGAAACCCGTGTCCCATTCCTTCATCTCCGGCCA
CCGTCTCGCCACCAACCTCTACACCTGCACTCGCAGCTATACCTAAATCAATCGGGTCTAATCGAGAAACCGAACCGAAC
AACAACTCAAATCCTCGAACCGGGTTAAGACCAGGAGTTATAATCGGAATCATAGTCGGAGATATCGCCGGAATCGGAAT
CCTCGCTCTTATCTTCTTCTACGTTTATAAATACAAAAACAACAAGACAGTGGAGAAGAAGAACAATCATAGCCTAGAAG
CTCATGAAGCTAAAGACACAACTTCGTTATCACCATCATCATCAACAACTACATCTTCTTCATCTCCAGAACAATCAAGC
AGATTTGCAAAATGGTCATGTCTCCGTAAGAATCAAGAAACCGATGAAACCGAAGAAGAAGACGAAGAAAATCAACGGTC
AGGAGAGATTGGAGAGAATAAGAAAGGGACTTTAGTAACCATTGATGGAGGAGAGAAAGAGCTTGAAGTTGAAACTTTGC
TTAAGGCTTCTGCTTACATTTTAGGAGCCACTGGTTCGAGTATAATGTACAAGACTGTTCTTGAGGACGGTACGGTTCTC
GCGGTTCGTCGGTTAGGTGAGAATGGTTTGAGTCAACAACGCCGGTTTAAAGACTTTGAGGCACATATTCGAGCTATTGG
TAAATTGGTTCACCCGAATTTGGTACGTCTTCGTGGATTCTATTGGGGCACCGACGAGAAATTGGTCATTTACGATTTTG
TTCCTAACGGCAGTCTCGTCAACGCCCGTTACAGGAAAGGAGGGTCTTCGCCGTGCCATTTACCGTGGGAGACTCGGCTC
AAGATAGTAAAAGGTTTGGCTCGTGGGCTTGCTTACCTCCACGACAAGAAACATGTGCACGGTAACTTGAAGCCTAGTAA
CATACTCTTGGGCCAAGATATGGAGCCCAAGATCGGAGATTTCGGGCTCGAAAGGCTTCTCGCCGGGGATACTAGCTATA
ACCGAGCTAGTGGATCATCTCGGATTTTCAGTAGCAAGCGATTGACAGCATCCTCGCGTGAATTTGGTACCATCGGGCCC
ACACCGAGCCCAAGTCCAAGCTCCGTTGGGCCCATATCTCCCTATTGCGCACCCGAGTCGCTCCGCAATCTCAAACCAAA
CCCGAAATGGGATGTGTTTGGGTTTGGAGTGATCCTCCTCGAGCTGCTCACGGGAAAAATAGTGTCGATAGACGAGGTGG
GGGTAGGAAATGGGCTGACCGTAGAGGACGGGAACCGGGCGCTAATAATGGCTGATGTAGCGATCCGCTCCGAATTGGAA
GGCAAAGAGGACTTTTTACTTGGCCTTTTCAAATTGGGATATAGTTGTGCATCTCAAATTCCACAAAAGAGACCGACCAT
GAAAGAGGCGTTAGTAGTGTTTGAAAGATATCCTATTAGCTCATCGGCTAAGAGTCCATCGTACCATTACGGACACTATT
AA
>12736079_protein_ID_12736080
MKTQSASPFFFVSFFFFFFFFSSLFLLSSALNSDGVLLLSFKYSVLLDPLSLLQSWNYDHDNPCSWRGVLCNNDSRVVTL
SLPNSNLVGSIPSDLGFLQNLQSLNLSNNSLNGSLPVEFFAADKLRFLDLSNNLISGEIPVSIGGLHNLQTLNLSDNIFT
GKLPANLASLGSLTEVSLKNNYFSGEFPGGGWRSVQYLDISSNLINGSLPPDFSGDNLRYLNVSYNQISGEIPPNVGAGF
PQNATVDFSFNNLTGSIPDSPVYLNQKSISFSGNPGLCGGPTRNPCPIPSSPATVSPPTSTPALAAIPKSIGSNRETEPN
NNSNPRTGLRPGVIIGIIVGDIAGIGILALIFFYVYKYKNNKTVEKKNNHSLEAHEAKDTTSLSPSSSTTTSSSSPEQSS
RFAKWSCLRKNQETDETEEEDEENQRSGEIGENKKGTLVTIDGGEKELEVETLLKASAYILGATGSSIMYKTVLEDGTVL
AVRRLGENGLSQQRRFKDFEAHIRAIGKLVHPNLVRLRGFYWGTDEKLVIYDFVPNGSLVNARYRKGGSSPCHLPWETRL
KIVKGLARGLAYLHDKKHVHGNLKPSNILLGQDMEPKIGDFGLERLLAGDTSYNRASGSSRIFSSKRLTASSREFGTIGP
TPSPSPSSVGPISPYCAPESLRNLKPNPKWDVFGFGVILLELLTGKIVSIDEVGVGNGLTVEDGNRALIMADVAIRSELE
GKEDFLLGLFKLGYSCASQIPQKRPTMKEALVVFERYPISSSAKSPSYHYGHY*
>12739224_construct_ID_Bin2A2-28716-HY2
GTGCGCTCTCATATTTCTCACATTTTCGTAGCCGCAAGACTCCTTTCAGATTCTTACTTGCAGCTATGGGTAAAGAGAAG
TTTCACATTAACATTGTGGTCATTGGTCATGTTGATTCTGGAAAATCGACCACAACTGGTCACTTGATCTATAAGCTTGG
TGGTATTGACAAGCGTGTCATCGAGAGGTTCGAGAAGGAGGCTGCTGAGATGAACAAGAGGTCCTTCAAGTACGCATGGG
TGTTGGACAAACTTAAGGCCGAGCGTGAGCGTGGTATTACCATCGATATTGCTCTATGGAAGTTCGAGACCACCAAGTAC
TACTGCACAGTCATTGATGCCCCAGGACATCGTGATTTCATCAAGAACATGATTACTGGTACCTCCCAGGCTGATTGTGC
TGTTCTTATCATTGACTCCACCACTGGAGGTTTTGAGGCTGGTATCTCTAAGGATGGTCAGACCCGTGAGCACGCTCTTC
TTGCTTTCACCCTTGGTGTCAAGCAGATGATTTGCTGTTGTAACAAGATGGATGCCACCACCCCCAAATACTCCAAGGCT
AGGTACGATGAAATCATCAAGGAGGTGTCTTCATACCTGAAGAAGGTCGGATACAACCCTGACAAAATCCCATTTGTGCC
AATCTCTGGATTCGAGGGAGACAACATGATTGAGAGGTCAACCAACCTTGACTGGTACAAGGGACCAACTCTTCTTGAGG
CTCTTGACCAGATCAACGAGCCCAAGAGGCCATCAGACAAGCCCCTTCGTCTTCCACTTCAGGATGTCTACAAGATTGGT
GGTATTGGAACGGTGCCAGTGGGACGTGTTGAGACTGGTATGATCAAGCCTGGTATGGTTGTTACCTTTGCTCCCACAGG
GTTGACCACTGAGGTTAAGTCTGTTGAGATGCACCACGAGTCTCTTCTTGAGGCACTTCCCGGTGACAATGTTGGATTCA
ATGTCAAGAATGTTGCTGTCAAGGATCTTAAGAGAGGATACGTTGCCTCTAACTCCAAGGATGATCCAGCTAAGGGTGCC
GCCAACTTCACCTCCCAGGTCATCATCATGAACCACCCTGGTCAGATTGGTAACGGTTACGCCCCAGTTCTCGATTGCCA
CACCTCTCACATTGCAGTCAAGTTCTCTGAGATCTTGACCAAGATTGACAGGCGTTCTGGTAAGGAGATTGAGAAGGAGC
CCAAGTTTTTGAAGAATGGTGACGCTGGTATGGTTAAGATGACCCCAACCAAGCCCATGGTTGTTGAGACTTTCTCCGAG
TACCCACCTTTGGGACGTTTCGCTGTTAGGGACATGAGGCAGACCGTTGCTGTTGGTGTTATTAAGAGCGTGGACAAGAA
GGACCCAACTGGAGCCAAGGTCACCAAGGCTGCAGTGAAGAAGGGTGCCAAATGATGAGACTTTCGTTATGATCGACTCT
CTTATGGTTTTCTTTGGTTCTTAAAACTTTGATGGCGTTTGAGCCTTTTTCTTTTTTCTCTTTATTTCTGTGACTTTCTC
TCTCCCTCCTTTTTGGATATCTCTGAGACTTTTTATTATGGTTTTCAATTATGCAGTTTCCGGATAATTTTGCTTGAAAC
T
>12739224_protein_ID_12739226
MGKEKFHINIVVIGHVDSGKSTTTGHLIYKLGGIDKRVIERFEKEAAEMNKRSFKYAWVLDKLKAERERGITIDIALWKF
ETTKYYCTVIDAPGHRDFIKNMITGTSQADCAVLIIDSTTGGFEAGISKDGQTREHALLAFTLGVKQMICCCNKMDATTP
KYSKARYDEIIKEVSSYLKKVGYNPDKIPFVPISGFEGDNMIERSTNLDWYKGPTLLEALDQINEPKRPSDKPLRLPLQD
VYKIGGIGTVPVGRVETGMIKPGMVVTFAPTGLTTEVKSVEMHHESLLEALPGDNVGFNVKNVAVKDLKRGYVASNSKDD
PAKGAANFTSQVIIMNHPGQIGNGYAPVLDCHTSHIAVKFSEILTKIDRRSGKEIEKEPKFLKNGDAGMVKMTPTKPMVV
ETFSEYPPLGRFAVRDMRQTVAVGVIKSVDKKDPTGAKVTKAAVKKGAK*
>13489977_construct_ID_YP0134
CAGTCGGTTCTCGAGTCATCGCCAAGGACCCACTTCATCATTTTACAAACCAAGCAAGACTAATCCAACAAAAAAATAGT
CCACAAAAAGATTTTTACAGATGGCGATTAACAGATCTTTACTTTTGATTCTTCTTTTCATCTCTGTTTCTCTATCGACG
GCGAGGATCTTACCCGGAGAGTTTGTTCCAGTCATCTTCTCCGGAGAGATCCCTCCTGTTTCTAAGTCGGCGGTGGTTGG
TTGCGGAGGCGAGCAGGAGACCAAGACGGAATATTCTTCTTTTGTTCCTGAAGTTGTCGCCGGAAAGTTCGGGTCCTTGG
TGTTGAATGCTCTTCCGAAAGGGAGTCGTCCGGGGTCTGGACCCAGCAAGAAAACTAACGACGTCAAGACTTAGCACTAT
TCTTTCTAGAGTTTTCTGTCCTAATTCTTACTTCTTTCTTTTTTTGTTCTTTAGAGATTCTTTGATTTTTCGTTTTCAAA
TAGAGATTATTGTAAATGTTACATGTATTACAGAAATTTACAGTAGAAGTTTAGGAAAAATGAGGATTTTATTTGGTAAT
GTAAGTCGAAATGATCAAGACTTAGACTATCATCTTGTATCGTTTCATCAATATTTCTTTGATAAACGTTAATCAGCTTT
TTAATTTCTATGATTATGTATCAATTTTATTTAGACTAAGAAAGTCTTTTAAGTTAAACGCATAAAAGAGTCAAGGATAC
CATTTGAATTT
>13489977_protein_ID_13489978
MLFRKGVVRGLDPARKLTTSRLSTILSRVFCPNSYFFLFLFFRDSLIFRFQIEIIVNVTCITEIYSRSLGKMRILFGNVS
RNDQDLDYHLVSFHQYFFDKR*
>13491988_construct_ID_YP0016
GTCTCCTCTTCGGATAATCCTATCCTTCTCTTCCTATAAATACCTCTCCACTCTTCCTCTTCCTCCACCACTACAACCAC
CGCAACAACCACCAAAAACCCTCTCAAAGAAATTTCTTTTTTTTCTTACTTTCTTGGTTTGTCAAATATGGTCAGCCATC
CAATGGAGAAAGCTGCAAATGGTGCGTCTGCGTTGGAAACGCAGACGGGTGAGTTAGATCAGCCGGAACGGCTTCGTAAG
ATCATATCGGTGTCTTCCATTGCCGCCGGTGTACAGTTCGGTTGGGCTTTACAGTTATCTCTGTTGACTCCTTACGTGCA
GCTACTCGGAATCCCACATAAATGGGCTTCTCTGATTTGGCTCTGTGGTCCAATCTCCGGTATGCTTGTTCAGCCTATCG
TCGGTTACCACAGTGACCGTTGCACCTCAAGATTCGGCCGTCGTCGTCCCTTCATCGTCGCTGGAGCTGGTTTAGTCACC
GTTGCTGTTTTCCTTATCGGTTACGCTGCCGATATAGGTCACAGCATGGGCGATCAGCTTGACAAACCGCCGAAAACGCG
AGCCATAGCGATATTCGCTCTCGGGTTTTGGATTCTTGACGTGGCTAACAACACCTTACAAGGACCCTGCAGAGCTTTCT
TGGCTGATTTATCAGCAGGGAACGCTAAGAAAACGCGAACCGCAAACGCGTTTTTCTCGTTTTTCATGGCGGTTGGAAAC
GTTTTGGGTTACGCTGCGGGATCTTACAGAAATCTCTACAAAGTTGTGCCTTTCACGATGACTGAGTCATGCGATCTCTA
CTGCGCAAACCTCAAAACGTGTTTTTTCCTATCCATAACGCTTCTCCTCATAGTCACTTTCGTATCTCTCTGTTACGTGA
AGGAGAAGCCATGGACGCCAGAGCCAACAGCCGATGGAAAAGCCTCCAACGTTCCGTTTTTCGGAGAAATCTTCGGAGCT
TTCAAGGAACTAAAAAGACCCATGTGGATGCTTCTTATAGTCACTGCACTAAACTGGATCGCTTGGTTCCCTTTCCTTCT
CTTCGACACTGATTGGATGGGCCGTGAGGTGTACGGAGGAAACTCAGACGCAACCGCAACCGCAGCCTCTAAGAAGCTTT
ACAACGACGGAGTCAGAGCTGGTGCTTTGGGGCTTATGCTTAACGCTATTGTTCTTGGTTTCATGTCTCTTGGTGTTGAA
TGGATTGGTCGGAAATTGGGAGGAGCTAAAAGGCTTTGGGGTATTGTTAACTTCATCCTCGCCATTTGCTTGGCCATGAC
GGTTGTGGTTACGAAACAAGCTGAGAATCACCGACGAGATCACGGCGGCGCTAAAACAGGTCCACCTGGTAACGTCACAG
CTGGTGCTTTAACTCTCTTCGCCATCCTCGGTATCCCCCAAGCCATTACGTTTAGCATTCCTTTTGCACTAGCTTCCATA
TTTTCAACCAATTCCGGTGCCGGCCAAGGACTTTCCCTAGGTGTTCTGAATCTAGCCATTGTCGTCCCTCAGATGGTAAT
ATCTGTGGGAGGTGGACCATTCGACGAACTATTCGGTGGTGGAAACATTCCAGCATTTGTGTTAGGAGCGATTGCGGCAG
CGGTAAGTGGTGTATTGGCGTTGACGGTGTTGCCTTCACCGCCTCCGGATGCTCCTGCCTTCAAAGCTACTATGGGATTT
CATTGAATTTTAGCAGTGGTTGTTTGGCTCTCTTTCTCTCATAAAACAGTAGTGTTGTGCAAATCCTACATAAAGAAAAA
AGAAAAGGAAATTAAACTCATTGGGTTGGTTTGTATTTTACCTAAACCCACGAAGTTCCTTTTTCTTTTTGTAACTCAAT
TTAAATTTGGAGTATATTTTACTTTTTGTTACCTTCAAGGCTTCAATATTACGACTTCATTGTTCGG
>13491988_protein_ID_13491989
MVSHPMEKAANGASALETQTGELDQPERLRKIISVSSIAAGVQFGWALQLSLLTPYVQLLGIPHKWASLIWLCGPISGML
VQPIVGYHSDRCTSRFGRRRPFIVAGAGLVTVAVFLIGYAADIGHSMGDQLDKPPKTRAIAIFALGFWILDVANNTLQGP
CRAFLADLSAGNAKKTRTANAFFSFFMAVGNVLGYAAGSYRNLYKVVPFTMTESCDLYCANLKTCFFLSITLLLIVTFVS
LCYVKEKPWTPEPTADGKASNVPFFGEIFGAFKELKRPMWMLLIVTALNWIAWFPFLLFDTDWMGREVYGGNSDATATAA
SKKLYNDGVRAGALGLMLNAIVLGFMSLGVEWIGRKLGGAKRLWGIVNFILAICLANTVVVTKQAENHRRDHGGAKTGPP
GNVTAGALTLFAILGIPQAITFSIPFALASIFSTNSGAGQGLSLGVLNLAIVVPQMVISVGGGPFDELFGGGNIPAFVLG
AIAAAVSGVLALTVLPSPPPDAPAFKATMGFH*
>13580795_construct_ID_YP0087
TTTAGGGTTTATTCTTCATTGCTTGAGCTTCCTTCTCTTCTTCTTCTTCAAGCCGCGGCTAAAGATCCCTACTTCTCTCG
ACACTTATAGAGTTTCAGTCATGGCCGCCTCCGCAGAAATCGACGCTGAGATTCAACAGCAGCTTACCAATGAGGTTAAG
CTCTTCAACCGTTGGAGCTTTGATGACGTTTCGGTTACGGATATTAGTCTTGTGGACTACATTGGTGTTCAGCCATCGAA
GCACGCAACTTTTGTTCCCCATACTGCTGGACGATACTCTGTGAAGAGGTTCAGAAAGGCGCAGTGCCCAATTGTTGAGA
GGCTCACTAACTCTCTCATGATGCACGGAAGAAACAATGGTAAGAAGTTGATGGCTGTCAGGATCGTCAAGCATGCCATG
GAGATTATCCACCTCTTGTCTGACTTGAACCCGATTCAAGTTATCATTGATGCCATTGTTAACAGTGGTCCACGTGAAGA
TGCTACCAGGATTGGATCTGCTGGTGTGGTTAGGAGGCAGGCTGTTGATATCTCTCCTCTAAGACGTGTGAACCAAGCGA
TCTTCTTGCTTACAACTGGTGCACGTGAAGCTGCCTTTAGAAACATCAAGACAATCGCTGAGTGCCTTGCTGATGAACTC
ATCAATGCTGCAAAGGGATCTTCCAACAGCTATGCCATCAAGAAGAAAGATGAGATTGAGAGAGTTGCTAAGGCCAATCG
TTAAGGGATCTCCCTTTCCTCTAAGTTTGCATTATATCAAAGAGTTTTTGTGTTGTTTCCATTAGCTTTGGATATGTTTC
AGATGATCTCTCTATCTTTAATGAAATTTTGACGCTTATAATCGACTTGGGATCTTGA
>13580795_protein_ID_13580797
MAASAEIDAEIQQQLTNEVKLFNRWSFDDVSVTDISLVDYIGVQPSKHATFVPHTAGRYSVKRFRKAQCPIVERLTNSLM
MHGRNNGKKLMAVRIVKHAMEIIHLLSDLNPIQVIIDAIVNSGPREDATRIGSAGVVRRQAVDISPLRRVNQAIFLLTTG
AREAAFRNIKTIAECLADELINAAKGS_SNSYAIKKKDEIERVAKANR*
>13601936_construct_ID_YP0108
ATCATAAACCCACCGAGACGATGTCTCTCATCATCGTCTTCTTCTTCTTCTCACTCTTGCTCACATCCAATGGACAGTTC
TTCGACGAGAGCAAGAACTATGAAGGCTCCTCCGATCTCGTTGACCTTCAATACCACTTGGGTCCGGTCATATCCTCGCC
GGTGACGAGTCTCTACATCATTTGGTACGGCCGATGGAACCCAACTCACCAATCTATAATCCGAGACTTTCTCTACTCTG
TCTCTGCACCGGCACCGGCTCAGTACCCGTCAGTATCCAACTGGTGGAAGACAGTGAGGCTATACAGAGACCAGACAGGT
TCCAACATCACCGACACTCTTGTCTTATCCGGAGAGTTCCACGACTCAACGTACTCTCATGGATCTCATCTCACTCGCTT
CTCTGTTCAGTCTGTGATCAGAACTGCCTTGACTTCCAAGTTACCACTAAACGCTGTAAACGGCTTGTACTTAGTCTTGA
CCTCGGATGATGTAGAGATGCAAGAGTTCTGCAGAGCGATTTGCGGGTTTCATTACTTCACTTTCCCAAGCGTTGTGGGT
GCAACCGTACCGTATGCTTGGGTGGGCAACAGTGAGAGACAGTGTCCAGAAATGTGTGCGTACCCATTTGCACAGCCTAA
GCCATTTCCGGGGAGCGGGTTTGTAGCCAGAGAGAAGATGAAACCGCCAAATGGAGAGGTAGGAATCGATGGGATGATCA
GTGTGATAGCTCATGAGCTGGCAGAAGTGTCGAGTAACCCGATGTTAAACGGATGGTATGGAGGAGAGGACGCGACAGCA
CCGACAGAGATAGCGGATTTATGTTTGGGAGTGTATGGGTCAGGAGGAGGAGGAGGCTATATGGGAAGTGTGTATAAGGA
TAGGTGGAGGAATGTGTATAATGTGAAGGGCGTTAAAGGAAGAAAGTATCTAATTCAATGGGTTTGGGATCTTAATAGGA
ACAGATGCTTTGGACCAAACGCTATGAATTAGAGACTATCATGTTTGTTACCTCTTTTCACCAAAGCCTTGAGCTTGAAG
CTTGGGGAAACCTGTATATGGTTTATCTTTTCCTTGCCTAGTCGATTCTATGCATTTGATTGTTTAAGACT
>13601936_protein_ID_13601938
MSLIIVFFFFSLLLTSNGQFFDESKNYEGSSDLVDLQYHLGPVISSPVTSLYIIWYGRWNPTHQSIIRDFLYSVSAPAPA
QYPSVSNWWKTVRLYRDQTGSNITDTLVLSGEFHDSTYSHGSHLTRFSVQSVIRTALTSKLPLNAVNGLYLVLTSDDVEM
QEFCRAICGFHYFTFPSVVGATVPYAWVGNSERQCPEMCAYPFAQPKPFPGSGFVAREKMKPPNGEVGIDGMISVIAHEL
AEVSSNPMLNGWYGGEDATAPTEIADLCLGVYGSGGGGGYMGSVYKDRWRNVYNVKGVKGRKYLIQWVWDLNRNRCFGPN
AMN*
>13604221_construct_ID_YP0110
ATCAATCTTACATCCAAAACTTAAAGTATTCTTACATCCAAAAACAAAAAAAATATGGCAAAGTCTCTTCTCATAGTAAT
GCTCATGTCTATAGTAATGTTTTACATGGCTCGTCCAATTTTCTCCCAAAAAATTAATCCATATTTAGAGGTGATGCCAA
AAGATGTGACCATATCTCCATCTTCAAATTTTGATTACGTCGAAGCTCCCGATGAAGCTCCATTCGAAGAAGCTGATTCA
CCAGCAATGGAATATGACATGGAGCTTGCTCACCATTATTCGGACAAACAGCTCAAGTTTCTTGAGGCTTGCTCTGAAAA
GCCGAGTTCAAAATGCGGAAATGAGGTTTTCAAGAACATGTTAAATGAGACGATGCTAATTACAGAGGAATGTTGTCGTG
ATATATTGAAGATGGGCAAAGATTGCCATCTAGGATTGGTTAAACTCATATTTGCCACATATGAGTATAAAAATATTGCA
TCTAAGGGCATTCCAAAGAGCAAACAAACATGGAACGAATGTGTCCATAGAGTGGGGAGCAAGATTGGTGCTCCGGTCTC
TTTTGAACAATGAACTAATATTTCCGTGTATTGATGTGTCTATGCGTTTTTGTAATTTGATTATTACTAATATAAAGCAA
CTGCTACTATTTT
>13604221_protein_ID_13604222
MAKSLLIVMLMSIVMFYMARPIFSQKINPYLEVMPKDVTISPSSNFDYVEAPDEAPFEEADSPANEYDMELAHHYSDKQL
KFLEACSEKPSSKCGNEVFKNMLNETMLITEECCRDILKMGKDCHLGLVKLIFATYEYKNIASKGIPKSKQTWNECVHRV
GSKIGAPVSFEQ*
>13609100_construct_ID_YP0082
ACAGTTCTCAGATAAATACTAAACTCACTGTTAAAACTTTCTCAACAAAGCTTCCTGTTTCTCTACAAATGGCATCTGCT
CTCGCTCTTAAGAGACTCCTATCATCCTCCATCGCTCCACGTTCCCGTAGTGTTCTTCGTCCAGCTGTTTCCTCTCGCCT
CTTCAACACCAACGCCGTTAGGAGCTACGACGACGACGGCGAAAATGGAGACGGCGTTGATTTATATCGCCGCTCTGTTC
CTCGCCGCCGTGGTGATTTCTTCTCAGATGTGTTTGATCCGTTTTCGCCGACGAGGAGCGTTAGTCAAGTGCTGAATCTG
ATGGACCAGTTCATGGAGAATCCTCTGTTATCAGCTACTCGTGGCATGGGAGCTTCAGGAGCTCGTCGTGGTTGGGATAT
AAAAGAGAAAGACGATGCTCTGTACCTGAGAATCGACATGCCTGGGCTGAGCAGAGAGGATGTGAAGCTGGCTTTGGAGC
AGGACACTCTGGTGATTAGAGGAGAAGGAAAAAACGAGGAAGATGGTGGCGAGGAAGGAGAGAGCGGTAATCGGAGATTC
ACAAGCAGGATTGGATTACCGGATAAGATTTACAAGATCGATGAGATTAAGGCGGAGATGAAGAACGGAGTGTTGAAAGT
TGTGATCCCGAAGATGAAAGAACAAGAGAGAAATGATGTTCGTCAGATCGAGATCAACTAAAAACGTCGACGTTTTTTTC
TGTTCTAGTTTTGTTGATAGGTCTTTGAATAAGAAGTGTGTGTAGTTTGGCACGGTCGATGTTGAGTCATGTAGTCTCTA
AAGACTAAAAGGTTATATGTTTCTTTCTTG
>13609100_protein_ID_13609102
MASALALKRLLSSSIAPRSRSVLRPAVSSRLFNTNAVRSYDDDGENGDGVDLYRRSVPRRRGDFFSDVFDPFSPTRSVSQ
VLNLMDQFMENPLLSATRGMGASGARRGWDIKEKDDALYLRIDMPGLSREDVKLALEQDTLVIRGEGKNEEDGGEEGESG
NRRFTSRIGLPDKIYKIDEIKAEMKNGVLKVVIPKMKEQERNDVRQIEIN*
>13609583_construct_ID_Bin1-344414-HY2
ATTTTTAACGCTCACTGGATTTATAAGTAGAGATTTTTTGTGTCTCACAAAAACAAAAAAATCATCGTGAAACGTTCGAA
GGCCATTTTCTTTGGACGACCATCGGCGTTAAGGAGAGAGCTTAGATCTCGTGCCGTCGTGCGACGTTGTTTTCCGGCTT
GATCAAAATGGGGTTGTCATTCGGAAAGTTGTTCAGCAGGCTCTTTGCGAAGAAAGAGATGCGTATTCTGATGGTTGGTC
TCGATGCTGCTGGTAAGACGACTATCCTCTACAAGCTCAAACTTGGAGAGATCGTCACCACTATTCCAACCATTGGGTTC
AACGTTGAGACTGTTGAATACAAGAACATCAGCTTCACCGTGTGGGATGTTGGGGGTCAAGACAAGATCCGTCCATTGTG
GAGACATTACTTCCAGAACACACAGGGACTTATCTTTGTTGTGGACAGCAATGATCGTGACCGTGTTGTTGAAGCCAGGG
ACGAGCTTCACAGGATGCTGAATGAGGATGAATTGAGGGATGCAGTTCTGCTTGTATTTGCTAACAAGCAAGATCTTCCC
AACGCGATGAACGCTGCTGAGATAACTGACAAGCTTGGGCTTCATTCTCTTCGTCAACGACACTGGTACATTCAGAGCAC
ATGTGCCACCTCTGGAGAAGGACTCTATGAGGGACTTGACTGGCTCTCCAACAACATCGCAAGCAAGGCATAGATGGAAT
GTTAGCCAGATTCCTCTTCTGCTTGTTTGGTTTACAAATCAAAGACAGAGGTCTGTTTCTCTAGTACTAAAAGATTTATT
ATTATATTCTTCTTCGTCACTTATCTCAAACGCAGATCATTTTACACTTTGTACTTCCCCTTCAATAACTTGTTACTTCT
CTCGTTTGCTTCCTGAATTTGAGTATATCATTTTTACATCTGCTTTTCATCAAAGCATAAAGCATCTTTCGAAACAAAAA
TTGAACCGAATTTTTCTGTAAACTGATCAAATGTG
>13609583_protein_ID_13609584
MGLSFGKLFSRLFAKKEMRILMVGLDAAGKTTILYKLKLGEIVTTIPTIGFNVETVEYKNISFTVWDVGGQDKIRPLWRH
YFQNTQGLIFVVDSNDRDRVVEARDELHRMLNEDELRDAVLLVFANKQDLPNAMNAAEITDKLGLHSLRQRHWYIQSTCA
TSEGLYEGLDWLSNNIASKA*
>13609817_construct_ID_YP0094
GCAGCAGCAAATACTATCATCACCCATCTCCTTAGTTCTATTTTATAATTCCTCTTCTTTTTGTTCATAGCTTTGTAATT
ATAGTCTTATTTCTCTTTAAGGCTCAATAAGAGGAGATGGGTGAAACCGCTGCCGCCAATAACCACCGTCACCACCACCA
TCACGGCCACCAGGTCTTTGACGTGGCCAGCCACGATTTCGTCCCTCCACAACCGGCTTTTAAATGCTTCGATGATGATG
GCCGCCTCAAAAGAACTGGGACTGTTTGGACCGCGAGCGCTCATATAATAACTGCGGTTATCGGATCCGGCGTTTTGTCA
TTGGCGTGGGCGATTGCACAGCTCGGATGGATCGCTGGCCCTGCTGTGATGCTATTGTTCTCTCTTGTTACTCTTTACTC
CTCCACACTTCTTAGCGACTGCTACAGAACCGGCGATGCAGTGTCTGGCAAGAGAAACTACACTTACATGGATGCCGTTC
GATCAATTCTCGGTGGGTTCAAGTTCAAGATTTGTGGGTTGATTCAATACTTGAATCTCTTTGGTATCGCAATTGGATAC
ACGATAGCAGCTTCCATAAGCATGATGGCGATCAAGAGATCCAACTGCTTCCACAAGAGTGGAGGAAAAGACCCATGTCA
CATGTCCAGTAATCCTTACATGATCGTATTTGGTGTGGCAGAGATCTTGCTCTCTCAGGTTCCTGATTTCGATCAGATTT
GGTGGATCTCCATTGTTGCAGCTGTTATGTCCTTCACTTACTCTGCCATTGGTCTAGCTCTTGGAATCGTTCAAGTTGCA
GCGAATGGAGTTTTCAAAGGAAGTCTCACTGGAATAAGCATCGGAACAGTGACTCAAACACAGAAGATATGGAGAACCTT
CCAAGCACTTGGAGACATTGCCTTTGCGTACTCATACTCTGTTGTCCTAATCGAGATTCAGGATACTGTAAGATCCCCAC
CGGCGGAATCGAAAACGATGAAGAAAGCAACAAAAATCAGTATTGCCGTCACAACTATCTTCTACATGCTATGTGGCTCA
ATGGGTTATGCCGCTTTTGGAGATGCAGCACCGGGAAACCTCCTCACCGGTTTTGGATTCTACAACCCGTTTTGGCTCCT
TGACATAGCTAACGCCGCCATTGTTGTCCACCTCGTTGGAGCTTACCAAGTCTTTGCTCAGCCCATCTTTGCCTTTATTG
AAAAATCAGTCGCAGAGAGATATCCAGACAATGACTTCCTCAGCAAGGAATTTGAAATCAGAATCCCCGGATTTAAGTCT
CCTTACAAAGTAAACGTTTTCAGGATGGTTTACAGGAGTGGCTTTGTCGTTACAACCACCGTGATATCGATGCTGATGCC
GTTTTTTAACGACGTGGTCGGGATCTTAGGGGCGTTAGGGTTTTGGCCCTTGACGGTTTATTTTCCGGTGGAGATGTATA
TTAAGCAGAGGAAGGTTGAGAAATGGAGCACGAGATGGGTGTGTTTACAGATGCTTAGTGTTGCTTGTCTTGTGATCTCG
GTGGTCGCCGGGGTTGGATCAATCGCCGGAGTGATGCTTGATCTTAAGGTCTATAAGCCATTCAAGTCTACATATTGATG
ATTATGGACCATGAACAACAGAGAGAGTTGGTGTGTAAAGTTTACCATTTCAAAGAAAACTCCAAAAATGTGTATATTGT
ATGTTGTTCTCATTTCGTATGGTCTCATCTTTGTAATAAAATTTAAAACTTATGTTATAAATTATAAAACCGTGTGTTTT
C
>13609817_protein_ID_13609818
MGETAAANNHRHHHHHGHQVFDVASHDFVPPQPAFKCFDDDGRLKRTGTVWTASAHIITAVIGSGVLSLAWAIAQLGWIA
GPAVMLLFSLVTLYSSTLLSDCYRTGDAVSGKRNYTYMDAVRSILGGFKFKICGLIQYLNLFGIAIGYTIAASISMMAIK
RSNCFHKSGGKDPCHMSSNPYNIVFGVAEILLSQVPDFDQIWWISIVAAVMSFTYSAIGLALGIVQVAANGVFKGSLTGI
SIGTVTQTQKIWRTFQALGDIAFAYSYSVVLIEIQDTVRSPPAESKTMKKATKISIAVTTIFYMLCGSMGYAAFGDAAPG
NLLTGFGFYNPFWLLDIANAAIVVHLVGAYQVFAQPIFAFIEKSVAERYPDNDFLSKEFEIRIPGFKSPYKVNVFRMVYR
SGFVVTTTVISMLMPFFNDVVGILGALGFWPLTVYFPVEMYIKQRKVEKWSTRWVCLQMLSVACLVISVVAGVGSIAGVM
LDLKVYKPFKSTY*
>13610584_construct_ID_YP0128
ATAATCCAAACACCAAAAACAAAATGGAGAAATTGCTCGTGATCTCTTTGCTACTACTGATCTCAACATCAGTTACAACT
TCACAATCCGTGACCGATCCAATAGCTTTCCTCCGATGTCTCGATAGACAACCAACGGACCCAACAAGTCCTAACTCCGC
CGTTGCTTACATCCCAACAAACTCTTCTTTCACCACTGTCCTCCGCAGCCGTATACCTAACCTCCGTTTCGACAAACCCA
CTACTCCAAAACCCATCTCCGTGGTGGCTGCCGCCACGTGGACACACATACAAGCTGCTGTAGGATGCGCACGTGAGCTC
TCTCTCCAAGTCAGGATCAGAAGTGGTGGCCACGACTTCGAAGGACTCTCTTACACTTCCACCGTCCCTTTCTTTGTTCT
CGACATGTTCGGTTTTAAAACCGTGGACGTAAATCTCACCGAGAGAACGGCTTGGGTTGATTCTGGTGCTACCCTCGGAG
AGCTTTACTATAGAATCTCTGAGAAGAGCAATGTTCTTGGATTTCCGGCGGGTTTGTCTACCACATTGGGCGTTGGTGGA
CACTTTAGCGGCGGAGGATACGGTAATCTGATGAGAAAGTATGGTTTGTCGGTGGATAACGTTTTCGGCTCCGGGATCGT
TGATTCGAACGGAAATATCTTCACCGATCGGGTTTCGATGGGGGAAGACCGTTTTTGGGCGATTCGTGGAGGTGGTGCAG
CGAGCTACGGTGTTGTCCTCGGCTACAAGATCCAGCTAGTACCGGTGCCTGAGAAAGTTACGGTTTTTAAAGTCGGAAAA
ACTGTCGGAGAAGGAGCCGTTGATCTTATAATGAAGTGGCAGAGTTTTGCTCATAGTACGGATCGGAATTTGTTCGTGAG
GTTAACTTTGACTTTAGTCAACGGTACGAAGCCTGGTGAGAATACGGTTTTAGCGACTTTCATTGGGATGTATTTAGGCC
GGTCGGATAAGCTGTTGACCGTGATGAACCGGGATTTCCCGGAGTTGAAGCTGAAGAAAACCGATTGTACCGAGATGAGA
TGGATCGATTCGGTTCTGTTTTGGGACGATTATCCGGTTGGTACACCGACTTCTGTGCTACTAAATCCGCTAGTCGCAAA
AAAGTTGTTCATGAAACGAAAATCGGACTACGTGAAGCGTCTGATTTCGAGAACCGATCTCGGTTTGATACTCAAGAAAT
TGGTAGAGGTTGAGAAAGTTAAAATGAATTGGAATCCGTATGGAGGAAGGATGGGTGAGATCCCGAGTTCGAGGACACCA
TTCCCACATAGAGCAGGCAATTTGTTCAACATTGAGTATATCATAGACTGGTCAGAAGCTGGAGATAATGTGGAGAAGAA
ATATTTGGCACTCGCGAATGAATTTTATAGATTCATGACCCCGTACGTGTCTAGTAATCCGAGGGAGGCGTTTTTGAATT
ACCGTGATCTTGACATAGGGTCAAGTGTTAAGTCTACGTACCAGGAAGGTAAAATCTACGGGGCTAAATATTTCAAGGAG
AATTTCGAGAGATTAGTGGATATTAAAACCACGATTGATGCGGAAAACTTTTGGAAAAACGAACAAAGCATTCCGGTTAG
AAGATAA
>13610584_protein_ID_13610586
MEKLLVISLLLLISTSVTTSQSVTDPIAFLRCLDRQPTDPTSPNSAVAYIPTNSSFTTVLRSRIPNLRFDKPTTPKPISV
VAAATWTHIQAAVGCARELSLQVRIRSGGHDFEGLSYTSTVPFFVLDMFGFKTVDVNLTERTAWVDSGATLGELYYRISE
KSNVLGFPAGLSTTLGVGGHFSGGGYGNLMRKYGLSVDNVFGSGIVDSNGNIFTDRVSMGEDRFWAIRGGGAASYGVVLG
YKIQLVPVPEKVTVFKVGKTVGEGAVDLIMKWQSFAHSTDRNLFVRLTLTLVNGTKPGENTVLATFIGMYLGRSDKLLTV
MNRDFPELKLKKTDCTEMRWIDSVLFWDDYPVGTPTSVLLNPLVAKKLFMKRKSDYVKRLISRTDLGLILKKLVEVEKVK
MNWNPYGGRMGEIPSSRTPFPHRAGNLFNIEYIIDWSEAGDNVEKKYLALANEFYRFMTPYVSSNPREAFLNYRDLDIGS
SVKSTYQEGKIYGAKYFKENFERLVDIKTTIDAENFWKNEQSIPVRR*
>13612879_construct_ID_YP0104
GTATCTATACTCATAAATCCTTTTGTCTAAAAATGGCGATGCTAGGTTTTTACGTAACGTTCATTTTCTTTCTTGTATGC
CTATTTACTTATTTCTTCCTCCAAAAGAAACCTCAAGGTCAGCCTATTCTCAAGAACTGGCCGTTCCTCAGGATGCTTCC
AGGAATGCTCCACCAAATCCCTCGTATCTACGACTGGACCGTCGAGGTGCTTGAGGCGACCAATCTAACTTTTTATTTCA
AAGGGCCATGGCTTAGTGGAACGGACATGTTGTTCACCGCCGATCCAAGGAATATTCATCACATACTAAGCTCAAACTTT
GGGAATTACCCTAAAGGACCTGAGTTCAAGAAGATCTTTGATGTTTTGGGAGAAGGAATCTTAACCGTTGATTTTGAGTT
GTGGGAGGAGATGAGGAAGTCAAATCACGCCCTATTCCACAATCAAGATTTCATCGAGCTCTCAGTAAGTAGCAATAAAA
GTAAGTTAAAAGAAGGTCTTGTTCCTTTTCTTGATAATGCTGCTCAGAAAAACATTATCATAGAATTACAAGATGTGTTC
CAGAGATTCATGTTTGATACTTCTTCAATTTTGATGACTGGTTACGATCCAATGTCACTATCCATCGAAATGCTGGAAGT
TGAGTTCGGTGAAGCTGCGGATATTGGCGAAGAAGCAATCTATTATAGACATTTCAAACCGGTGATCTTGTGGAGGCTTC
AAAACTGGATTGGTATTGGGCTTGAGAGGAAGATGAGAACAGCTTTGGCCACTGTCAATCGTATGTTTGCGAAGATCATA
TCTTCAAGAAGAAAAGAGGAGATAAGTCGCGCCAAAACGGAGCCATATTCCAAGGACGCGTTGACGTATTATATGAATGT
GGACACGAGCAAATATAAGCTCTTGAAACCTAATAAAGATAAGTTTATAAGAGATGTTATTTTTAGTCTAGTGTTAGCAG
GAAGGGACACCACAAGCTCAGTTCTCACTTGGTTCTTTTGGCTTCTTTCTAAGCATCCTCAAGTTATGGCCAAGCTCAGA
CATGAGATCAACACAAAGTTTGATAATGAAGATCTAGAGAAGCTCGTGTATCTGCATGCTGCATTGTCCGAATCAATGAG
ACTCTACCCGCCACTTCCCTTCAACCACAAGTCTCCTGCGAAGCCAGATGTACTTCCAAGCGGGCACAAAGTTGATGCAA
ATTCAAAGATCGTGATATGTATCTATGCATTGGGGAGGATGAGATCTGTATGGGGAGAAGACGCATTGGATTTCAAACCA
GAGAGATGGATTTCAGACAATGGAGGTCTAAGACATGAACCTTCATACAAGTTCATGGCTTTTAATTCTGGTCCGAGAAC
TTGCTTGGGTAAAAATCTAGCTCTCTTGCAGATGAAGATGGTAGCTCTGGAGATCATACGAAACTATGACTTTAAGGTCA
TTGAAGGTCACAAGGTCGAACCAATTCCTTCTATCCTTCTCCGTATGAAACATGGTCTTAAAGTCACAGTCACAAAGAAG
ATATGATTATTATGCTTGCTTGGCTTCTACGGCAACTATTACTATTTCCTTATTTAAATGTGTTACTTACTAGTTTGTTC
CCACGTTATAACTACTTGTATTACGTACTAAGTACGGTGTTTGTCCCACGTCATGCTCATAAATTAATTAATATCGTCAA
TAAAGTATTAGAGCATCCTCGTCCAT
>13612879_protein_ID_13612881
MAMLGFYVTFIFFLVCLFTYFFLQKKPQGQPILKNWPFLRMLPGMLHQIPRIYDWTVEVLEATNLTFYFKGPWLSGTDML
FTADPRNIHHILSSNFGNYPKGPEFKKIFDVLGEGILTVDFELWEEMRKSNHALFHNQDFIELSVSSNKSKLKEGLVPFL
DNAAQKNIIIELQDVFQRFMFDTSSILMTGYDPMSLSIEMLEVEFGEAADIGEEAIYYRHFKPVILWRLQNWIGIGLERK
MRTALATVNRMFAKIISSRRKEEISRAKTEPYSKDALTYYMNVDTSKYKLLKPNKDKFIRDVIFSLVLAGRDTTSSVLTW
FFWLLSKHPQVMAKLRHEINTKFDNEDLEKLVYLHAALSESMRLYPPLPFNHKSPAKPDVLPSGHKVDANSKIVICIYAL
GRMRSVWGEDALDFKPERWISDNGGLRHEPSYKFMAFNSGPRTCLGKNLALLQMKMVALEIIRNYDFKVIEGHKVEPIPS
ILLRMKHGLKVTVTKKI*
>13612919_construct_ID_YP0075
AAAAAAAGAACCGTTTTTTCTTTCTATGGCTCCAAAACTCTGAGACAGAGCAAAAAGAJAGATAAGTGAGTGAAAAAATG
GCAACGGTCACGATTCTCTCACCCAAATCGATTCCAAAGGTCACTGATTCCAAATTCGGAGCTAGGGTTTCTGATCAGAT
CGTCAATGTCGTAAAATGCGGCAAATCCGGCCGGAGATTGAAGTTAGCGAAGCTGGTCTCAGCGGCTGGATTGTCACAGA
TCGAACCAGACATCAACGAAGACCCGATTGGTCAATTCGAGACTAATAGCATTGAAATGGAAGATTTCAAGTATGGATAT
TACGATGGAGCTCATACTTACTATGAAGGAGAAGTTCAAAAGGGAACATTTTGGGGAGCAATTGCTGATGACATTGCTGC
TGTGGATCAAACTAATGGGTTTCAAGGTTTGATCTCTTGTATGTTTCTTCCTGCTATAGCTCTTGGGATGTATTTTGATG
CTCCGGGTGAGTACTTGTTCATAGGTGCAGCGTTATTCACGGTAGTGTTCTGTATAATAGAGATGGATAAACCTGACCAG
CCACACAACTTCGAGCCTCAGATATACAAATTGGAGAGAGGAGCTCGTGACAAGCTCATTAATGACTACAACACAATGAG
CATTTGGGACTTTAATGACAAATATGGTGATGTATGGGATTTCACCATTGAGAAAGATGATATCGCCACACGATAAGATA
ATGGATTGTGATCTCGTTATAATCATGACTTTTGATGTAAACTGTTTTATAAAATTGATGAATGAACGGGGTACAATGTG
TATAATATTGATTGTTCATTC
>13612919_protein_ID_13612921
MATVTILSPKSIPKVTDSKFGARVSDQIVNVVKCGKSGRRLKLAKLVSAAGLSQIEPDINEDPIGQFETNSIEMEDFKYG
YYDGAHTYYEGEVQKGTFWGAIADDIAAVDQTNGFQGLISCMFLPAIALGMYFDAPGEYLFIGAALFTVVFCIIEMDKPD
QPHNFEPQIYKLERGARDKLINDYNTMSIWDFNDKYGDVWDFTIEKDDIATR*
>13613553_construct_ID_YP0060
AAACCTTTCTCTTCTCTGCTAACGAGAAAACAAAAGCTATCGTCTTTGCTACTACTACTACTACTATTATTACATTGAAT
CCTTTGTGTTCTTCTTCTTCAGCTGCTACTTTGTTCGAGTGCTTTCTTACATGCCGTCGGAGATTGTTGACAGGAAAAGG
AAGTCTCGTGGAACACGAGATGTAGCTGAGATTCTAAGGCAATGGAGAGAGTACAATGAGCAGATTGAGGCAGAATCTTG
TATCGATGGTGGTGGTCCAAAATCAATCCGAAAGCCTCCTCCAAAAGGTTCGAGGAAGGGTTGTATGAAAGGTAAAGGTG
GACCTGAAAACGGGATTTGTGACTATAGAGGAGTTAGACAGAGGAGATGGGGTAAATGGGTTGCTGAGATCCGTGAGCCA
GACGGAGGTGCTAGGTTGTGGCTCGGTACTTTCTCCAGTTCATATGAAGCTGCATTGGCTTATGACGAGGCGGCCAAAGC
TATATATGGTCAGTCTGCCAGACTCAATCTTCCCGAGATCACAAATCGCTCTTCTTCGACTGCTGCCACTGCCACTGTGT
CAGGCTCGGTTACTGCATTTTCTGATGAATCTGAAGTTTGTGCACGTGAGGATACAAATGCAAGTTCAGGTTTTGGTCAG
GTGAAACTAGAGGATTGTAGCGATGAATATGTTCTCTTAGATAGTTCTCAGTGTATTAAAGAGGAGCTGAAAGGAAAAGA
GGAAGTGAGGGAAGAACATAACTTGGCTGTTGGTTTTGGAATTGGACAGGACTCGAAAAGGGAGACTTTGGATGCTTGGT
TGATGGGAAATGGCAATGAACAAGAACCATTGGAGTTTGGTGTGGATGAAACGTTTGATATTAATGAGCTATTGGGTATA
TTAAACGACAACAATGTGTCTGGTCAAGAGACAATGCAGTATCAAGTGGATAGACACCCAAATTTCAGTTACCAAACGCA
GTTTCCAAATTCTAACTTGCTCGGGAGCCTCAACCCTATGGAGATTGCTCAACCAGGAGTTGATTATGGATGTCCTTATG
TGCAGCCCAGTGATATGGAGAACTATGGTATTGATTTAGACCATCGCAGGTTCAATGATCTTGACATACAGGACTTGGAT
TTTGGAGGAGACAAAGATGTTCATGGATCTACATAAGATTTCAAATTTCGTTTGACTGGCCTAAGTTTGTGATTCTGCTC
CGAGACGGTGTAGCTGTTACTAGCTAGAAGCTGCCCTTCTTTGAAGCTACTGATACTTTCTGATATTAATGGTTGTGAGA
CGTAGTACATGTAGTTAGGTAATGTAGGACAAGTTCAAATATGATTCCTTCTTTCTTTTTCTTGTGAATACATATGACAT
ATGAAGAAGTTCAAACGTTGGGT
>13613553_protein_ID_13613554
MPSEIVDRKRKSRGTRDVAEILRQWREYNEQIEAESCIDGGGPKSIRKPPPKGSRKGCMKGKGGPENGICDYRGVRQRRW
GKWVAEIREPDGGARLWLGTFSSSYEAALAYDEAAKAIYGQSARLNLPEITNRSSSTAATATVSGSVTAFSDESEVCARE
DTNASSGFGQVKLEDCSDEYVLLDSSQCIKEELKGKEEVREEHNLAVGFGIGQDSKRETLDAWLMGNGNEQEPLEFGVDE
TFDINELLGILNDNNVSGQETMQYQVDRHPNFSYQTQFPNSNLLGSLNPMEIAQPGVDYGCPYVQPSDMENYGIDLDHRR
FNDLDIQDLDFGGDKDVHGST*
>13613954_construct_ID_YP0102
AATCACACAAATCCCTTTTTTGGTTTCTCCAAATCTTCAAATCTTCTTCAATCATCACCATGGTACGTTTTAGTAACAGT
CTTGTAGGAATACTCAACTTCTTCGTCTTCCTTCTCTCGGTTCCCATACTCTCAACCGGAATCTGGCTCAGCCTTAAAGC
CACGACGCAATGCGAGAGATTCCTCGACAAACCCATGATCGCTCTCGGTGTTTTCCTCATGATAATCGCAATCGCTGGAG
TCGTTGGATCTTGTTGCAGAGTGACGTGGCTTCTCTGGTCCTATCTCTTTGTGATGTTCTTCTTAATCCTCATCGTCCTC
TGTTTCACCATCTTTGCCTTCGTTGTCACTAGTAAAGGCTCCGGCGAAACTATCCAAGGAAAAGCTTATAAGGAGTATAG
GCTCGAGGCTTACTCTGATTGGTTGCAGAGGCGTGTGAACAACGCTAAGCATTGGAACAGCATTAGAAGCTGTCTTTATG
AGAGCAAGTTCTGTTATAACTTGGAGTTAGTCACTGCTAATCACACTGTTTCTGATTTCTACAAAGAAGATCTCACTGCT
TTTGAGTCTGGTTGCTGCAAGCCCTCTAATGACTGTGACTTCACCTACATAACTTCAACAACTTGGAATAAAACATCAGG
AACACATAAAAACTCAGATTGCCAACTTTGGGACAACGAAAAGCATAAGCTTTGCTACAATTGCAAAGCCTGCAAGGCCG
GTTTTCTCGACAACCTCAAGGCCGCATGGAAAAGAGTTGCTATTGTCAACATCATTTTCCTTGTACTCCTCGTTGTCGTC
TACGCTATGGGATGTTGCGCTTTCCGAAACAACAAAGAAGATAGATATGGCCGTTCCAATGGTTTCAACAATTCTTGATT
TGCGCCGGTTCAAGCTAGACTTTGATTTTTCATTAATACATCATATTACATTTATGATTAGAACAAAACAGCTTTCPAAA
TTTAAGAAACAGTAGAATGGAAGAATATTGAATTAGTATAGTTGTTGATGTGTTTGGATTTCTTCTGTTGATTTGTGTTT
GGACAACAGAGGATTCTTCAGATCTTTATTACAGATTGTTGTGTTTGAAGAATCTTCTATATGAATCTTCACTTCTGACT
TCTG
>13613954_protein_ID_13613956
MVRFSNSLVGILNFFVFLLSVPILSTGIWLSLKATTQCERFLDKPMIALGVFLMIIAIAGVVGSCCRVTWLLWSYLFVMF
FLILIVLCFTIFAFVVTSKGSGETIQGKAYKEYRLEAYSDWLQRRVNNAKHWNSIRSCLYESKFCYNLELVTANNTVSDF
YKEDLTAFESGCCKPSNDCDFTYITSTTWNKTSGTHKNSDCQLWDNEKHKLCYNCKACKAGFLDNLKAAWKRVAIVNIIF
LVLLVVVYAMGCCAFRNNKEDRYGRSNGFNNS*
>13617784_construct_ID_YP0127
GAAACTTGTTTTCTCTTTCCCTTCTTCAATCAAAACCTATTTGCATGCTCTCAAACCCGAATTAAATCGACACTTTTCAG
TTTTTGTTTTAACAAGTAGAGTTTCCCAAAATATTGGATATATTTCTTTTTCAAATTTCGGAAAAGAAATGAGTTGCAAT
GGATGTAGAGTTCTTCGAAAAGGTTGCAGTGAAACATGCATCCTTCGTCCTTGCCTTCAATGGATCGAATCCGCCGAGTC
ACAAGGCCACGCCACCGTCTTCGTCGCTAAATTCTTTGGTCGTGCTGGTCTCATGTCTTTCATCTCCTCCGTACCTGAAC
TCCAACGTCCTGCTTTGTTTCAGTCGTTGTTGTTTGAAGCGTGTGGGAGAACGGTGAATCCGGTTAACGGAGCGGTTGGT
ATGTTGTGGACCAGGAACTGGCACGTATGCCAAGCGGCGGTTGAGACTGTTCTTCGCGGCGGAACTTTACGACCGATATC
AGATCTTCTTGAATCTCCGTCGTTGATGATCTCCTGTGATGAGTCTTCAGAGATTTGGCATCAAGACGTTTCAAGAAACC
AAACCCACCATTGTCGCTTCTCCACCTCCAGATCCACGACGGAGATGAAAGACTCTCTGGTTAACCGAAAACGATTGAAG
TCCGATTCGGATCTTGATCTCCAAGTGAACCACGGTTTAACCCTAACCGCTCCGGCTGTACCGGTTCCTTTTCTTCCTCC
GTCGTCGTTTTGTAAGGTGGTTAAGGGTGATCGTCCGGGAAGTCCATCGGAGGAATCTGTAACGACGTCGTGTTGGGAAA
ATGGGATGAGAGGAGATAATAAACAAAAAAGAAACAAAGGAGAGAAAAAGTTATTGAACCTTTTTGTTTAAAACCGACGA
CGCAAAACACTCAAAGATTTTGAGGCTCTCTTTTTTAGGGTTTTGAGTGGGAATGGATATTTAGTTAATGATTTTTCTCT
ATCGAGAAATATGATAAAATTTTGGGG
>13617784_protein_ID_13617786
MSCNGCRVLRKGCSETCILRPCLQWIESAESQGHATVFVAKFFGRAGLMSFISSVPELQRPALFQSLLFEACGRTVNPVN
GAVGMLWTRNWHVCQAAVETVLRGGTLRPISDLLESPSLMISCDESSEIWHQDVSRNQTHHCRFSTSRSTTEMKDSLVNR
KRLKSDSDLDLQVNHGLTLTAPAVPVPFLPPSSFCKVVKGDRPGSPSEESVTTSCWENGMRGDNKQKRNKGEKKLLNLFV
*
>13647840_construct_ID_YP0186
GAAAAACAAAAAAAAGGGGGAACAAGGGAGTTTCATGTTAAAAAAAAATGAAGCTCTCTTGTTTGGTTTTTCTCATAGTA
TCGTCTCTTGTTTCGAGTTCTCTTGCCACCGCTCCGCCCAACACATCTATATATGAAAGCTTTCTCCAATGTTTCAGCAA
TCAAACAGGTGCTCCTCCTGAGAAGTTATGCGACGTCGTTCTGCCTCAAAGCAGTGCCAGCTTCACTCCAACCCTACGTG
CCTACATCCGTAACGCTCGTTTCAACACTTCCACGTCCCCCAAACCTCTGCTCGTTATCGCGGCGCGTTCTGAGTGCCAC
GTCCAGGCCACCGTCCTCTGCACCAAATCTCTCAACTTCCAGCTCAAGACTCGCAGCGGCGGCCATGACTACGACGGCGT
TTCCTACATCTCTAACCGCCCTTTCTTCGTCCTCGACATGTCCTATCTCCGTAACATTACCGTCGATATGTCCGACGACG
GCGGCTCTGCTTGGGTTGGAGCCGGCGCTACTCTCGGCGAAGTTTATTACAACATTTGGCAGAGCAGCAAAACTCACGGC
ACTCACGGATTTCCCGCCGGTGTTTGTCCCACAGTAGGCGCTGGAGGTCACATTAGCGGCGGGGGCTACGGCAACATGAT
CAGAAAATACGGACTTTCCGTGGACTACGTCACGGACGCCAAAATCGTAGACGTGAACGGACGGATTCTCGATCGTAAAT
CGATGGGAGAGGATTTGTTTTGGGCGATTGGAGGCGGTGGTGGTGCGAGCTTCGGCGTGATCTTATCTTTCAAGATCAAA
CTCGTGCCTGTTCCTCCGAGGGTGACTGTTTTCAGAGTGGAGAAGACCCTAGTAGAAAACGCACTTGACATGGTCCATAA
ATGGCAGTTTGTTGCTCCCAAGACCAGCCCGGATCTCTTCATGAGGCTAATGTTGCAGCCAGTGACCCGGAACACGACTC
AGACGGTTCGCGCGTCGGTAGTTGCTCTGTTCTTGGGAAAACAGAGCGATCTCATGTCTCTGCTGACCAAGGAGTTCCCC
GAGCTTGGTCTGAAGCCGGAGAATTGCACGGAGATGACGTGGATACAGTCGGTGATGTGGTGGGCCAACAACGACAACGC
CACGGTGATTAAACCGGAGATCCTGCTGGATCGAAATCCGGATTCGGCGTCTTTCTTGAAAAGAAAATCGGATTACGTGG
AGAAAGAGATCAGCAAAGACGGTTTAGATTTCTTGTGTAAGAAGTTGATGGAGGCTGGGAAGCTAGGGCTAGTGTTCAAT
CCATACGGAGGGAAAATGAGCGAAGTTGCTACGACGGCGACTCCGTTCCCACACAGGAAGAGGCTTTTCAAGGTCCAGCA
TTCGATGAACTGGAAAGACCCGGGCACTGATGTTGAAAGCAGTTTCATGGAAAAGACGAGAAGCTTCTACAGCTACATGG
CTCCTTTCGTGACCAAGAATCCAAGACACACGTATCTCAACTACAGGGATCTTGATATCGGGATCAACAGCCATGGCCCA
AACAGTTACAGAGAAGCTGAGGTTTACGGGAGAAAGTATTTCGGAGAGAATTTTGATCGGTTGGTCAAAGTCAAAACAGC
CGTGGATCCAGAAAACTTTTTCAGAGATGAACAAAGTATACCTACCTTGCCTACCAAGCCATCCTCGAGTTAG
>13647840_protein_ID_13647841
MKLSCLVFLIVSSLVSSSLATAPPNTSIYESFLQCFSNQTGAPPEKLCDVVLPQSSASFTPTLRAYIRNARF
NTSTSPKPLLVIAARSECHVQATVLCTKSLNFQLKTRSGGHDYDGVSYISNRPFFVLDMSYLRNITVDMSD
DGGSAWVGAGATLGEVYYNIWQSSKTHGTHGFPAGVCPTVGAGGHISGGGYGNMIRKYGLSVDYVTDAKIV
DVNGRILDRKSMGEDLFWAIGGGGGASFGVILSFKIKLVPVPPRVTVFRVEKTLVENALDMVHKWQFVAPK
TSPDLFMRLMLQPVTRNTTQTVRASVVALFLGKQSDLMSLLTKEFPELGLKPENCTEMTWIQSVMWWANND
NATVIKPEILLDRNPDSASFLKRKSDYVEKEISKDGLDFLCKKLMEAGKLGLVFNPYGGKMSEVATTATPF
PHRKRLFKVQHSMNWKDPGTDVESSFMEKTRSFYSYMAPFVTKNPRHTYLNYRDLDIGINSHGPNSYREAE
VYGRKYFGENFDRLVKVKTAVDPENFFRDEQSIPTLPTKPSSS*
>13614559_construct_ID_YP0024
GATCAAGAAAACTCGTCTCCTACAAAAATCCCAGAAGACAAGAGATTGGTTCTTCTTTTGCATCATTCTTACAAAATCCC
CAAAATCATTCGALACCCCTGAGTATTCTCCTTAACTCTAAGAAATAAATTTCTGAATGGATGCATCGTCTTCACCGTCT
CCTTCCGAGGAAAGCTTGAAGCTTGAGCTTGATGATCTTCAGAAACAGCTGAACAAAAAGCTGAGATTCGAAGCATCCGT
TTGTTCTATTCATAATCTTCTCCGTGATCACTACTCTTCTTCCTCTCCTTCTCTCCGCAAACAGTTCTATATAGTTGTAT
CTCGTGTCGCTACGGTTCTTAAGACAAGATATACAGCTACTGGATTTTGGGTTGCTGGACTGAGTCTTTTCGAAGAGGCT
GAGCGACTTGTCTCTGATGCTTCTGAGAAGAAACATTTGAAATCTTGCGTTGCTCAAGCTAAGGAGCAGTTAAGCGAAGT
AGATAATCAGCCAACAGAGAGCTCACAAGGTTATCTTTTTGAGGGACATCTTACGGTTGATCGTGAGCCGCCACAGCCTC
AGTGGCTAGTACAGCAGAATCTCATGTCTGCTTTCGCTTCTATCGTTGGTGGTGAATCCTCTAATGGTCCTACTGAAAAC
ACTATTGGGGAAACTGCTAACTTGATGCAAGAACTTATCAATGGTCTTGACATGATCATTCCAGATATACTAGATGATGG
TGGACCACCAAGAGCTCCACCGGCAAGTAAAGAAGTTGTAGAGAAACTCCCAGTCATTATTTTCACCGAGGAATTGCTTA
AAAAGTTTGGAGCAGAGGCAGAATGTTGCATCTGCAAGGAGAATCTAGTTATTGGCGACAAGATGCAGGAATTGCCATGC
AAGCACACATTTCACCCTCCTTGCCTAAAGCCTTGGCTGGACGAGCATAACTCTTGCCCTATATGCCGCCATGAATTACC
AACAGACGATCAGAAATACGAAAACTGGAAAGAGAGAGAGAAAGAGGCCGAAGAAGAGAGGAAGGGCGCAGAGAATGCTG
TCCGCGGAGGTGAATATATGTACGTTTAAATTTCAATCAGTTATGGCACACTCCCATTGTCTTTCCTTGAAACATCTCCG
AATTGTTGTTCATCATTCACAATTATAAATCCCATTTTACATATAGATTCAATGTCTTTTGTATGAAAGCTTATAATAAC
AACACAGACTTCTTTACTT
>13614559_protein_ID_13614560
MDASSSPSPSEESLKLELDDLQKQLNKKLRFEASVCSIHNLLRDHYSSSSPSLRKQFYIVVSRVATVLKTRYTATGFWVA
GLSLFEEAERLVSDASEKKHLKSCVAQAKEQLSEVDNQPTESSQGYLFEGHLTVDREPPQPQWLVQQNLMSAFASIVGGE
SSNGPTENTIGETANLMQELINGLDMIIPDILDDGGPPRAPPASKEVVEKLPVIIFTEELLKKFGAEAECCICKENLVIG
DKMQELPCKHTFHPPCLKPWLDEHNSCPICRHELPTDDQKYENWKEREKEAEEERKGAENAVRGGEYMYV*
>13614841_construct_ID_CR13 (GFP-ER)
TTCGTACTACTACTACCACCACATTTCTTTAGCTCAACCTTCATTACTAATCTCCTTTTAAGGTTTCTTTCGTGAATCAG
ATCGGAAAAATGGAATCTTTTTTGTTCACATCTGAATCCGTCAACGAGGGACATCCCGACAAGCTTTGTGATCAGATCTC
CGACGCTATCCTCGATGCTTGCCTTGAACAAGACCCTGAGAGCAAAGTTGCTTGTGAGACTTGTACCAAGACTAACATGG
TCATGGTTTTTGGAGAAATCACCACCAAGGCTAACGTTGATTACGAGCAGATTGTTCGTAAAACATGCCGTGAGATTGGA
TTCGTCTCTGCTGACGTTGGTCTAGATGCTGACAATTGCAAGGTTCTGGTTAACATTGAGCAACAGAGTCCTGACATTGC
ACAAGGTGTTCATGGTCATCTCACCAAGAAGCCAGAGGAGGTTGGAGCTGGTGACCAAGGTCACATGTTTGGGTATGCTA
CTGATGAGACTCCTGAGCTCATGCCTCTTACTCACGTTCTCGCTACTAAGCTTGGAGCTAAACTCACTGAAGTTCGCAAG
AATGGAACTTGCCCTTGGTTGAGGCCAGATGGTAAGACTCAAGTCACTATTGAGTACATCAACGAAAGCGGAGCCATGGT
TCCTGTACGTGTCCACACTGTTCTCATCTCAACACAGCATGACGAGACTGTGACTAACGATGAGATCGCAGCTGATCTTA
AGGAGCATGTGATCAAGCCAGTGATCCCAGAGAAATACCTTGATGAGAAAACCATCTTCCATCTCAACCCATCTGGTCGT
TTTGTTATCGGAGGTCCTCATGGAGATGCAGGGCTTACCGGCCGTAAGATCATCATCGATACTTATGGTGGTTGGGGTGC
ACACGGAGGTGGTGCTTTCTCTGGAAAGGACCCAACCAAGGTTGACAGGAGTGGGGCTTACATCGTTAGGCAAGCAGCTA
AGAGCATTGTAGCCAGTGGGCTAGCGAGGCGGGTCATTGTGQAAGTCTCGTATGCCATTGGTGTCCCTGAGCCATTGTCT
GTGTTCGTGGACAGTTATGGAACAGGAAAGATACCAGACAAGGAGATTCTTGAGATTGTGAAGGAGAGTTTTGATTTCAG
GCCAGGTATGATCTCCATTAACTTGGATCTGAAGAGAGGAGGTAATGGTAGGTTCTTGAAGACTGCTGCCTATGGTCACT
TTGGAAGGGACGATGCTGATTTCACCTGGGAGGTAGTCAAGCCACTCAAGTCTAACAAGGTCCAAGCTTGAAACCTGTCA
GCCTCTGTTTCACTTCTGTCCAGAATCAGTCTTGTTCTCTGTATTTTAGGCTCTTTCTGCCTCTTTAGTTTCAACTCTGA
GATGGGTTTATTCATTTTGTTTTCAACTTTGAAGAAAAAAGCTAAGCAGCTGGGAATTTATATAATTATTTATATGGTAT
TCTTGTGCTAAGAAAGTTAAATTCATAATATGTATTTCTTACTTATTTTGAGAAGAAAATCATATAAGAGAAT
>13614841_protein_ID_13614842
MESFLFTSESVNEGHPDKLCDQISDAILDACLEQDPESKVACETCTKTNMVMVFGEITTKANVDYEQIVRKTCREIGFVS
ADVGLDADNCKVLVNIEQQSPDIAQGVHGHLTKKPEEVGAGDQGHMFGYATDETPELMPLTHVLATKLGAKLTEVRKNGT
CPWLRPDGKTQVTIEYINESGAMVPVRVHTVLISTQHDETVTNDEIAADLKEHVIKPVIPEKYLDEKTIFHLNPSGRFVI
GGPHGDAGLTGRKIIIDTYGGWGAHGGGAFSGKDPTKVDRSGAYIVRQAAKSIVASGLARRVIVQVSYAIGVPEPLSVFV
DSYGTGKIPDKEILEIVKESFDFRPGMISINLDLKRGGNGRFLKTAAYGHFGRDDADFTWEVVKPLKSNKVQA*
>13617054_construct_ID_YP0117
ACTCAACACAAACTCTTTACGAATACTTTTAAGTATGGCTTCTTCTTCTGCAACCAAGTTTGTTGATCTGTTCCCATGTC
TTTTCTTAGCTTGCCTCTTCGTGTTCACATACTCAAACAACCTCGTCGTGGCTGAAAATTCCAACAAAGTGAAGATCAAT
CTTTACTATGAATCACTTTGTCCCTATTGTCAAAATTTCATTGTTGATGATCTAGGTAAAATCTTTGACTCCGATCTCCT
CAAAATCACCGATCTCAAGCTCGTTCCATTCGGTAACGCTCATATCTCCAATAATCTGACTATTACTTGCCAGCATGGTG
AAGAGGAATGCAAACTTAACGCTCTCGAAGCTTGCGGTATAAGAACTTTGCCCGATCCGAAATTGCAGTACAAGTTCATA
CGCTGCGTTGAAAAAGATACGAATGAATGGGAATCATGTGTTAAAAAATCTGGACGTGAGAAAGCCAATCATGATTGTTA
CAATGGTGATCTCTCTCAAAAGCTGATACTTGGGTATGCAAAACTGACCTCGAGTTTGAAGCCAAAACATGAATACGTAC
CATGGGTCACACTCAACGGCAAACCACTCTATGACAATTACCATAATTTGGTCGCACAAGTCTGCAAAGCGTACAAAGGA
AAGGATCTCCCAAAACTATGCAGTTCCTCGGTCTTGTATGAGAGGAAAGTGTCAAAGTTTCAAGTCTCCTATGTAGATGA
AGCTATCAATTAATAAGTTAATTAACAAACTTCTTATTGAAACTAAGATGGATCTAATCTTTATGCTATAAGTGGAATGA
TAAATAAAGACGTTTTATCTGAACTTTT
>13617054_protein_ID_13617056
MASSSATKFVDLFPCLFLACLFVFTYSNNLVVAENSNKVKINLYYESLCPYCQNFIVDDLGKIFDSDLLKITDLKLVPFG
NAHISNNLTITCQHGEEECKLNALEACGIRTLPDPKLQYKFIRCVEKDTNEWESCVKKSGREKAINDCYNGDLSQKLILG
YAKLTSSLKPKHEYVPWVTLNGKPLYDNYHNLVAQVCKAYKGKDLPKLCSSSVLYERKVSKFQVSYVDEAIN*
>13619323_construct_ID_YP0111
ACAAAATATCATAAACATATAAACATAAACGCCAATCGCAGCTTTTGTACTTTTGGCGGTTTACAATGGAGAAAGGTTTG
ACGATGTCTTGTGTTTTGGTGGTGGTTGCATTCTTAGCCATGGTTCATGTCTCTGTTTCAGTTCCGTTCGTAGTGTTTCC
TGAAATCGGAACACAATGTTCTGATGCTCCAAATGCTAACTTCACACAGCTTCTCAGTAACCTCTCTAGCTCACCTGGCT
TTTGCATAGAATTGGCGAGGGAAATCCAATAGGCGCTTCATGGTTAATACCACTTACACAAACAAGCGGAAGTAGCGTGT
GATAAGGTGACGCAGATGGAAGAGTTGAGTCAAGGATACAACATTGTTGGAAGAGCTCAGGGGAGCTTAGTGGCTCGAGG
CTTAATCGAGTTCTGCGAAGGTGGGCCTCCTGTTCACAACTATATATCCTTGGCTGGTCCTCATGCTGGCACCGCCGATC
TTCTTCGGTGTAATACTTCTGGCTTAATTTGTGACATAGCAAATGGGATAGGCAAGGAAAATCCCTACAGCGACTTTGTT
CAAGATAATCTTGCTCCTAGTGGTTATTTCAAAAACCCTAAAAATGTGACAGGGTACCTGAAAGACTGTCAGTATCTACC
TAAGCTTAACAATGAGAGACCATACGAAAGAAACACAACTTACAAAGACCGTTTCGCAAGTTTACAGAACCTGGTTTTTG
TCCTGTTTGAGAACGATACGGTTATTGTTCCAAAAGAGTCATCTTGGTTCGGGTTTTATCCGGATGGTGACTTAACACAT
GTTCTCCCTGTTCAAGAGACAAAGCTCTATATAGAAGATTGGATAGGTCTGAAAGCATTGGTTGTTGCTGGAAAAGTGCA
GTTTGTGAATGTAACCGGTGACCACTTAATAATGGCGGACGAAGATCTCGTCAAATACGTCGTACCTCTTCTCCAGGATC
AACAGTCTGCCCCACCAAGACTCAACCGCAAGACCAAGGAGCCCTTGCATCCTTAAAATGAGCAAATAGTTCAATCGCTA
TACTAATTCATCCAATGTCGAATAAGCTCAGTGATGATTGTGTGACACAATAATCCTTCTTCTTATATGAATAATAAAAG
CATACTATCT
>13619323_protein_ID_13619324
MEKGLTMSCVLVVVAFLAMVHVSVSVPFVVFPEIGTQCSDAPNANFTQLLSNLSSSPGFCIEIGEGNPIGASWLIPLTQQ
AEVACDKVTQMEELSQGYNIVGRAQGSLVARGLIEFCEGGPPVHNYISLAGPHAGTADLLRCNTSGLICDIANGIGKENP
YSDFVQDNLAPSGYFKNPKNVTGYLKDCQYLPKLNNERPYERNTTYKDRFASLQNLVFVLFENDTVIVPKESSWFGFYPD
GDLTHVLPVQETKLYIEDWIGLKALVVAGKVQFVNVTGDHLIMADEDLVKYVVPLLQDQQSAPPRLNRKTKEPLHP*
>12370095_construct_ID_YP0120
AGCACTCAACTTAAACTCTTTTAGTAACAATGGTTTCTTCTTCTTTAACCAAGCTTGTGTTCTTTGGTTGTCTCCTCCTG
CTCACATTCACGGACAACCTTGTGGCTGGAAAATCTGGCAAAGTGAAGCTCAATCTTTACTACGAATCACTTTGTCCCGG
TTGTCAGGAATTCATCGTCGATGACCTAGGTAAAATCTTTGACTACGATCTCTACACAATCACTGATCTCAAGCTGTTTC
CATTTGGTAATGCCGAACTCTCCGATAATCTGACTGTCACTTGCCAGCATGGTGAAGAGGAATGCAAACTAAACGCCCTT
GAAGCTTGCGCATTAAGAACTTGGCCCGATCAGAAATCACAATACTCGTTCATACGGTGCGTCGAAAGCGATACGAAAGG
CTGGGAATCATGTGTTAAAAACTCTGGACGTGAGAAAGCAATCAATGATTGTTACAATGGTGATCTTTCTAGAAAGCTGA
TACTTGGGTACGCAACCAAAACCAAGAATTTGAAGCCGCCACATGAATACGTACCATGGCTCACACTCAACGGCAAGCCA
CTCGATGACAGCGTACAAAGTACGGATGATCTCGTAGCTCAAATCTGCAATGCATACAAAGGAAAGACTACTCTCCCAAA
AGTTTGCAATTCATCCGCCTCAATGTCTAAGTCGCCTGAGAGGAAATGGAAGCTTCAAGTCTCTTATGCCAATAAAGCTA
CCAATTATTAAGTTAACTATCAAACTTCGTATTGAACTAAGATGGATTTAAGCTTTATGTTATAAGTGGAATGATGAATA
AAGGCCTGTTCTAAACTTTTATGGTTACGAATTGATGTATTAAAAAAGAACATGAAAAACGCCTGAACTGAACTACAAGT
ATTTTATATGACGTCTTATCGACGAAAGTGTTATGTAACTCGGTTTATC
>12370095_protein_ID_12370096
MVSSSLTKLVFFGCLLLLTFTDNLVAGKSGKVKLNLYYESLCPGCQEFIVDDLGKIFDYDLYTITDLKLFPFGNAELSDN
LTVTCQHGEEECKLNALEACALRTWPDQKSQYSFIRCVESDTKGWESCVKNSGREKAINDCYNGDLSRKLILGYATKTKN
LKPPHEYVPWVTLNGKPLDDSVQSTDDLVAQICNAYKGKTTLPKVCNSSASMSKSPERKWKLQVSYANKATNY*
>12385291_construct_ID_YP0261
aaacCCAACAACATAATTTCACATATCTCTCTTTCTTTCTCTTGAAGGAAAGACGAAGATCTCCAAGTCCCAAGTTGTTA
ACACAAGACGTAAACATGGGTCATCTTGGGTTCTTAGTTATGATTATGGTAGGAGTCATGGCTTCTTCTGTGAGCGGCTA
CGGTGGCGGTTGGATCAACGCTCACGCCACTTTTTACGGTGGTGGTGATGCTTCCGGCACAATGGGTGGTGCTTGTGGAT
ATGGTAATCTATATAGCCAAGGCTACGGGACGAGCACGGCGGCTCTAAGCACAGCTCTCTTCAACAATGGACTTAGCTGT
GGTTCTTGCTTTGAGATAAGATGTGAAAACGATGGTAAATGGTGTTTACCTGGCTCAATCGTTGTAACCGCTACAAACTT
CTGCCCGCCAAATAACGCGTTAGCGAACAATAATGGCGGTTGGTGTAATCCTCCTCTTGAACACTTTGACCTTGCTCAGC
CTGTTTTTCAACGCATTGCTCAGTACAGAGCTGGAATCGTCCCTGTTTCCTACAGAAGGGTTCCTTGCAGGAGAAGAGGA
GGAATAAGATTCACGATAAACGGCCACTCATACTTCAACCTTGTGCTGATCACAAACGTCGGTGGTGCCGGAGACGTTCA
CTCGGCGGCGATCAAGGGTTCAAGAACAGTGTGGCAAGCTATGTCAAGGAACTGGGGGCAAAATTGGCAAAGCAACTCTT
ACCTCAACGGTCAAGCACTTTCCTTTAAGGTCACCACCAGCGACGGCCGCACAGTTGTCTCCTTCAACGCCGCTCCTGCC
GGCTGGTCTTATGGCCAGACTTTTGCCGGTGGACAGTTCCGTTAAAAAGGGCAAGTTGGTTAATCTCTCTTCCATTTATC
TAAAGTAAACTCATTTGTGTGGTTATATTGGTCTCTTGAAAAAACTCGGTTATTGAGAGAGTGATGCGTCGAGGGCTCGG
TTTTGCAGAAGGCCTTGATGACGTCTAATCTTTTTTTGGACCTCTTTATTTTTCTTTCTTGAAACTAGTTTTTGTTAAGA
AAGAAAAAACAAGTTATAGTAGTTAATGTATTACTGATGCAGAGGTGGAGTTTTAACTACCACCCGCTAGTAGTAGTTAT
GAGTTTTTTATTTTAAGGTGTGAGAGAGAGATGGATTATCAAGATTTGTCAATTTTATTATGTTTGTTTGTAATAATACA
ATTCTTTACTCCAGTTAATGAAAATTGGGGGATTGATCACTTTT
>12385291_protein_ID_12385293
MGHLGFLVMIMVGVMASSVSGYGGGWINAHATFYGGGDASGTMGGACGYGNLYSQGYGTSTAALSTALFNNGLSCGSCFE
IRCENDGKWCLPGSIVVTATNFCPPNNALANNNGGWCNPPLEHFDLAQPVFQRIAQYRAGIVPVSYRRVPCRRRGGIRFT
INGHSYFNLVLITNVGGAGDVHSAAIKGSRTVWQAMSRNWGQNWQSNSYLNGQALSFKVTTSDGRTVVSFNAAPAGWSYG
QTFAGGQFR*
>12395532_construct_ID_YP0285
acAAATAAATACCTTTGTTTCCCTCTTCTTCTCCTTCACTCACAACATCTCAATTTCATTCTCTCTTCTCTCTCCAATTT
CACAACAATGGGAGTCAAAAGTTTCGTTGAAGGTGGGATTGCCTCTGTAATCGCCGOTTGCTCTACTCACCCTCTCGATC
TAATCAAGGTTCGTCTTCAGCTTCACGGTGAAGCACCTTCCACCACCACCGTCACTCTCCTCCGTCCAGCTCTCGCTTTC
CCCAATTCTTCTCCTGCAGCTTTCCTGGAAACGACTTCTTCAGTCCCCAAAGTAGGACCGATCTCACTCGGAATCAACAT
AGTCAAATCGGAAGGCGCCGCCGCGTTATTCTCAGGAGTCTCCGCTACACTTCTCCGTCAGACGTTATATTCCACCACCA
GGATGGGTCTATACGAAGTGCTTAAGAACAAATGGACTGATCCTGAGTCAGGGAAGTTGAATCTGAGTAGGAAGATCGGT
GCAGGGCTAGTCGCTGGTGGAATCGGAGCCGCCGTTGGAAATCCAGCTGACGTGGCGATGGTTAGGATGCAAGCTGACGG
GAGGTTACCTTTAGCGCAACGTCGTAACTACGCCGGAGTAGGAGACGCAATCAGGAGCATGGTTAAGGGAGAAGGCGTAA
CGAGCTTGTGGCGAGGCTCGGCGTTGACGATTAACCGAGCGATGATTGTGACGGCGGCTCAGCTAGCGTCTTACGATCAG
TTCAAGGAAGGGATATTGGAGAATGGTGTGATGAATGATGGGCTAGGGACTCACGTGGTAGCGAGTTTTGCGGCGGGGTT
TGTTGCTTCGGTTGCGTCTAATCCGGTGGATGTGATAAAGACGAGAGTGATGAATATGAAGGTGGGAGCGTACGACGGCG
CGTGGGATTGTGCGGTGAAGACGGTTAAAGCGGAAGGAGCCATGGCTCTTTATAAAGGCTTTGTTCCTACAGTTTGTAGG
CAAGGTCCTTTCACTGTTGTTCTCTTCGTTACGTTGGAGCAAGTTAGGAAGCTGCTTCGAGATTTTTGATACCATTCTTT
TATTGATGATGATGATGGCGACTATTTATATTGATTTATTCATTTTTGAAATAGTGAACACAAGAAGGAACTAGGAAGAG
GGGGATTCAATATATTTTTTGTTCAAGCATTGTTGTTAAATACAATTCAATTTTAGTTtC
>12395532_protein_ID_12395534
MGVKSFVEGGIASVIAGCSTHPLDLIKVRLQLHGEAPSTTTVTLLRPALAFPNSSPAAFLETTSSVPKVGPISLGINIVK
SEGAAALFSGVSATLLRQTLYSTTRMGLYEVLKNKWTDPESGKLNLSRKIGAGLVAGGIGAAVGNPADVAMVRMQADGRL
PLAQRRNYAGVGDAIRSMVKGEGVTSLWRGSALTINRANIVTAAQLASYDQFKEGILENGVMNDGLGTHVVASFAAGFVA
SVASNPVDVIKTRVMNMKVGAYDGAWDCAVKTVKAEGAMALYKGFVPTVCRQGPFTVVLFVTLEQVRKLLRDF*
>12575820_construct_ID_YP0216
TCTCTATAAATCCTTATATGTTTTACTTACATTCCTAAAGTTTTCAACTTTCTTGAGCTTCAAAAAGTACCTCCAATGGC
TTCTTCTGCATTTGCTTTTCCTTCTTACATAATAACCAAAGGAGGACTTTCAACTGATTCTTGTAAATCAACTTCTTTGT
CTTCTTCTAGATCTTTGGTTACAGATCTTCCATCACCATGTCTGAAACCCAACAACAATTCCCATTCAAACAGAAGAGCA
AAAGTGTGTGCTTCACTTGCAGAGAAGGGTGAATATTATTCAAACAGACCACCAACTCCATTACTTGACACTATTAACTA
CCCAATCCACATGAAAAATCTTTCTGTCAAGGAACTGAAACAACTTTCTGATGAGCTGAGATCAGACGTGATCTTTAATG
TGTCGAAAACCGGTGGACATTTGGGGTCAAGTCTTGGTGTTGTGGAGCTTACTGTGGCTCTTCATTACATTTTCAATACT
CCACAAGACAAGATTCTTTGGGATGTTGGTCATCAGTCTTATCCTCATAAGATTCTTACTGGGAGAAGAGGAAAGATGCC
TACAATGAGGCAAACCAATGGTCTCTCTGGTTTCACCAAACGAGGAGAGAGTGAACATGATTGCTTTGGTACTGGACACA
GCTCAACCACAATATCTGCTGGTTTAGGAATGGCGGTAGGAAGGGATTTGAAGGGGAAGAACAACAATGTGGTTGCTGTG
ATTGGTGATGGTGCGATGACGGCAGGACAGGCTTATGAAGCCATGAACAACGCCGGATATCTAGACTCTGATATGATTGT
GATTCTTAATGACAACAAGCAAGTCTCATTACCTACAGCTACTTTGGATGGACCAAGTCCACCTGTTGGTGCATTGAGCA
GTGCTCTTAGTCGGTTACAGTCTAACCCGGCTCTCAGAGAGTTGAGAGAAGTCGCAAAGGGTATGACAAAGCAAATAGGC
GGACCAATGCATCAGTTGGCGGCTAAGGTAGATGAGTATGCTCGAGGAATGATAAGCGGGACTGGATCGTCACTGTTTGA
AGAACTCGGTCTTTACTATATTGGTCCAGTTGATGGGCACAACATAGATGATTTGGTAGCCATTCTTAAAGAAGTTAAGA
GTACCAGAACCACAGGACCTGTACTTATTCATGTGGTGACGGAGAAAGGTCGTGGTTATCCTTACGCGGAGAGAGCTGAT
GACAAATACCATGGTGTTGTGAAATTTGATCCAGCAACGGGTAGACAGTTCAAAACTACTAATAAGACTCAATCTTACAC
AACTTACTTTGCGGAGGCATTAGTCGCAGAAGCAGAGGTAGACAAAGATGTGGTTGCGATTCATGCAGCCATGGGAGGTG
GAACCGGGTTAAATCTCTTTCAACGTCGCTTCCCAACAAGATGTTTCGATGTAGGAATAGCGGAACAACACGCAGTTACT
TTTGCTGCGGGTTTAGCCTGTGAAGGCCTTAAACCCTTCTGTGCAATCTATTCGTCTTTCATGCAGCGTGCTTATGACCA
GGTTGTCCATGATGTTGATTTGCAAAAATTACCGGTGAGATTTGCAATGGATAGAGCTGGACTCGTTGGAGCTGATGGTC
CGACACATTGTGGAGCTTTCGATGTGACATTTATGGCTTGTCTTCCTAACATGATAGTGATGGCTCCATCAGATGAAGCA
GATCTCTTTAACATGGTTGCAACTGCTGTTGCGATTGATGATCGTCCTTCTTGTTTCCGTTACCCTAGAGGTAACGGTAT
TGGAGTTGCATTACCTCCCGGAAACAAAGGTGTTCCAATTGAGATTGGGAAAGGTAGAATTTTAAAGGAAGGAGAGAGAG
TTGCGTTGTTGGGTTATGGCTCAGCAGTTCAGAGCTGTTTAGGAGCGGCTGTAATGCTCGAAGAACGCGGATTAAACGTA
ACTGTAGCGGATGCACGGTTTTGCAAGCCATTGGACCGTGCTCTCATTCGCAGCTTAGCTAAGTCGCACGAGGTTCTGAT
CACGGTTGAAGAAGGTTCCATTGGAGGTTTTGGCTCGCACGTTGTTCAGTTTCTTGCTCTCGATGGTCTTCTTGATGGCA
AACTCAAGTGGAGACCAATGGTACTGCCTGATCGATACATTGATCACGGTGCACCAGCTGATCAACTAGCTGAAGCTGGA
CTCATGCCATCTCACATCGCAGCAACCGCACTTAACTTAATCGGTGCACCAAGGGAAGCTCTGTTTTGAGAGTAAGAATC
TGTTGGCTAAAACATATGTATACAAACACTCTAAATGCAACCCAAGGTTTCTTCTAAGTACTGATCAGAATTCCCGCCGA
GAAGTCCTTTGGCAACAGCTATATATATTTACTAAGATTGTGAAGAGAAAGGCAAAGGCAAAGGTTGTGCAAAGATTAGT
ATTATGATAAAACTGGTATTTGTTTTGTAATTTTGTTTAGGATTGTGATGGAGATCGTGTTGTACAATAATCTAACATCT
TGTAAAAATCAATTACATCTCTTTGTGTA
>12575820_protein_ID_12575821
MASSAFAFPSYIITKGGLSTDSCKSTSLSSSRSLVTDLPSPCLKPNNNSHSNRRAKVCASLAEKGEYYSNRPPTPLLDTI
NYPIHMKNLSVKELKQLSDELRSDVIFNVSKTGGHLGSSLGVVELTVALHYIFNTPQDKILWDVGHQSYPHKILTGRRGK
MPTMRQTNGLSGFTKRGESEHDCFGTGHSSTTISAGLGMAVGRDLKGKNNNVVAVIGDGAMTAGQAYEAMNNAGYLDSDM
IVILNDNKQVSLPTATLDGPSPPVGALSSALSRLQSNPALRELREVAKGMTKQIGGPMHQLAAKVDEYARGMISGTGSSL
FEELGLYYIGPVDGHNIDDLVAILKEVKSTRTTGPVLIHVVTEKGRGYPYAERADDKYHGVVKFDPATGRQFKTTNKTQS
YTTYFAEALVAEAEVDKDVVAIHAAMGGGTGLNLFQRRFPTRCFDVGIAEQHAVTFAAGLACEGLKPFCAIYSSFMQRAY
DQVVHDVDLQKLPVRFANDRAGLVGADGPTHCGAFDVTFMACLPNMIVMAPSDEADLFNMVATAVAIDDRPSCFRYPRGN
GIGVALPPGNKGVPIEIGKGRILKEGERVALLGYGSAVQSCLGAAVMLEERGLNVTVADARFCKPLDRALIRSLAKSHEV
LITVEEGSIGGFGSHVVQFLALDGLLDGKLKWRPMVLPDRYIDHGAPADQLAEAGLMPSHIAATALNLIGAPREALF*
>12600234_construct_ID_YP0279
ATGTCGGCGTGTTTAAGCAGCGGAGGAGGAGGAGCAGCAGCATATAGTTTCGAGTTAGAAAAAGTGAAATCACCACCACC
ATCATCCTCAACAACAACAACAAGAGCTACTTCACCATCATCAACAATCTCCGAATCATCAAATTCACCACTCGCAATCT
CAACGAGAAAGCCAAGAACACAACGCAAAAGACCAAACCAGACTTACAACGAAGCAGCTACTCTTCTCTCTACTGCTTAT
CCCAACATCTTCTCCTCAAACTTGTCCTCTAAGCAAAAAACTCACTCTTCATCAAACTCTCACTTCTACGGGCCATTGCT
TAGTGACAACGACGACGCTTCTGATTTGCTTCTTCCTTATGAATCAATCGAAGAACCTGATTTTCTGTTTCATCCAACGA
TTCAAACGAAAACAGAGTTTTTCTCAGACCAGAAGGAAGTTAACTCCGGTGGAGATTGCTACGGTGGTGAAATCGAAAAG
TTTGATTTCTCCGACGAATTCGATGCTGAATCGATTCTCGATGAGGATATTGAAGAAGGAATCGATAGTATAATGGGGAC
TGTGGTGGAATCGAATTCAAATTCGGGGATTTATGAATCTAGGGTTCCGGGAATGATCAATCGCGGTGGAAGAAGTTCTT
CTAATCGGATTGGTAAACTAGAACAGATGATGATGATCAATTCATGGAATCGAAGCTCTAACGGATTCAATTTCCCGTTA
GGGCTTGGATTACGAAGTGCTCTCAGAGAAAACGACGACACAAAATTGTGGAAGATTCATACCGTTGATTTCGAACAGAT
CTCGCCGCGAATTCAAACTGTCAAAACCGAAACTGCAATCTCCACCGTTGATGAGGAGAAATCCGACGGTAAGAAGGTGG
TAATCTCTGGAGAGAAGAGTAATAAGAAGAAGAAGAAGAAGAAAATGACGGTGACGACGACATTGATTACGGAATCGAAA
AGCTTGGAAGATACGGAGGAGACGAGTTTGAAGAGAACAGGTCCGTTGTTGAAGCTTGATTACGACGGCGTTTTGGAAGC
TTGGTCTGATAAAACGTCGCCGTTTCCCGACGAGATTCAGGGATCGGAAGCTGTCGATGTCAATGCTAGATTAGCTCAGA
TTGATTTGTTCGGAGACAGTGGAATGCGAGAAGCAAGTGTTTTGAGGTACAAAGAGAAACGTCGAACTCGTCTTTTTTCG
AAGAAAATTCGATACCAAGTTCGCAAACTCAATGCTGATCAACGTCCTCGAATGAAGGGACGATTCGTGAGAAGGCCCAA
TGAGAGCACTCCAAGTGGACAAAGATAACAAGGATAAAAGAGCCTAGATTTATCTTATCTTTTTTTTTTTATCTTTTGTT
TATTCCTTGTTTTATTTTTGTTTCTAAAATTTTGGCACCCTCCTTTTTTGTTTCTTTTAAGTTATGGTCCCTTTTGGTTT
ATAATTTAGATTTTTTGATGAGGGGGAGATTTGATTGAGAAAGTGAGGGATCAAAACTAATAAAAGTTTTTGTTATTAAT
AGAAGAAACAGAGCTCTTGAGATT
>12600234_protein_ID_12600235
MSACLSSGGGGAAAYSFELEKVKSPPPSSSTTTTRATSPSSTISESSNSPLAISTRKPRTQRKRPNQTYNEPATLLSTAY
PNIFSSNLSSKQKTHSSSNSHFYGPLLSDNDDASDLLLPYESIEEPDFLFHPTIQTKTEFFSDQKEVNSGGDCYGGEIEK
FDFSDEFDAESILDEDIEEGIDSIMGTVVESNSNSGIYESRVPGMINRGGRSSSNRIGKLEQMMMINSWNRSSNGFNFPL
GLGLRSALRENDDTKLWKIHTVDFEQISPRIQTVKTETAISTVDEEKSDGKKVVISGEKSNKKKKKKKMTVTTTLITESK
SLEDTEETSLKRTGPLLKLDYDGVLEAWSDKTSPFPDEIQGSEAVDVNARLAQIDLFGDSGMREASVLRYKEKRRTRLFS
KKIRYQVRKLNADQRPRMKGRFVRRPNESTPSGQR*
>12603755_construct_ID_YP0080
ATTTTTGTTTTTATTTTTCTGATGTTACAATGGCAGACAAGATCTTCACTTTCTTCCTAATCTTGTCTTCGATCTCTCCT
CTCTTATGCTCTTCTTTGATCTCACCTCTTAATCTCTCACTTATTAGACAAGCAAATGTCCTTATCTCTCTAAAGCAAAG
TTTTGATTCCTATGATCCTTCTCTTGATTCATGGAACATTCCAAATTTCAACTCTCTATGTTCTTGGACTGGTGTTTCTT
GTGACAACTTGAATCAGTCTATTACTCGTCTAGACCTATCTAATCTCAACATCTCCGGCACTATCTCTCCGGAAATATCT
CGTCTTTCGCCGTCACTTGTTTTTCTTGACATTTCTTCTAACAGTTTCTCCGGTGAGCTTCCTAAAGAGATCTATGAGCT
CTCAGGCCTCGAAGTGTTAAACATCTCTAGCAATGTTTTTGAAGGAGAGCTGGAGACACGTGGGTTCAGTCAAATGACTC
AGCTTGTGACTCTTGACGCTTACGACAACAGCTTCAACGGATCACTTCCTCTGAGTCTAACCACACTCACTCGTCTCGAG
CACTTAGATCTTGGAGGAAACTACTTCGACGGTGAGATCCCTAGAAGCTATGGAAGTTTCTTGAGTCTCAAGTTTCTTTC
TTTATCTGGTAATGATCTCCGTGGGAGAATCCCTAACGAGCTAGCGAACATCACGACTTTGGTACAGCTTTACTTAGGTT
ACTACAACGATTACCGCGGTGGGATACCTGCAGATTTCGGGAGATTGATCAATCTTGTTCATTTGGATTTAGCTAATTGC
AGCTTGAAAGGATCAATTCCTGCAGAATTGGGGAATCTCAAGAACTTGGAGGTTCTGTTTCTTCAGACCAATGAGCTTAC
AGGCTCTGTTCCTCGAGAGTTAGGGAACATGACAAGCCTCAAGACTCTTGATCTCTCCAACAACTTTCTTGAAGGAGAGA
TTCCTCTAGAGCTATCTGGACTTCAAAAGCTTCAGTTGTTTAACCTCTTCTTCAACAGACTACACGGCGAGATCCCTGAG
TTCGTATCTGAGCTTCCTGATCTGCAAATACTCAAGCTTTGGCACAACAATTTCACCGGAAAGATTCCTTCGAAACTCGG
ATCAAACGGGAACTTGATCGAGATCGATTTGTCTACCAATAAACTCACAGGTTTGATCCCTGAGTCACTCTGTTTCGGAA
GAAGACTAAAGATTCTCATTCTCTTCAACAACTTCTTGTTCGGTCCTCTCCCTGAAGATCTTGGCCAATGTGAACCGCTA
TGGAGATTCCGTCTCGGACAGAACTTTCTGACAAGTAAGTTGCCAAAGGGTTTGATTTATTTGCCGAATCTTTCGCTTCT
TGAGCTTCAAAACAACTTTTTGACTGGAGAAATCCCCGAAGAAGAGGCGGGAAATGCGCAGTTTTCGAGCCTTACTCAGA
TCAATCTGTCCAACAACAGGTTATCCGGACCGATTCCTGGTTCAATCAGAAACCTCAGAAGCCTTCAGATTCTTCTTCTC
GGTGCAAACCGGTTATCGGGACAGATCCCTGGCGAAATCGGAAGTTTGAAGAGTCTTCTCAAGATTGACATGAGCAGAAA
CAACTTCTCAGGCAAGTTTCCTCCTGAGTTTGGTGATTGCATGTCACTCACATATTTAGATTTGAGTCACAACCAGATTT
CCGGTCAGATTCCGGTTCAGATATCGCAGATTCGGATTCTAAACTATCTGAATGTTTCTTGGAATTCCTTTAACCAAAGC
CTTCCCAACGAACTCGGATACATGAAGAGTTTAACATCAGCAGATTTCTCACACAACAACTTCTCCGGTTCAGTACCAAC
TTCAGGGCAATTCTCTTACTTCAACAACACGTCATTCCTTGGAAACCCTTTTCTCTGTGGATTTTCTTCAAACCCTTGCA
ACGGTTCCCAAAACCAATCTCAATCTCAGCTACTTAACCAGAACAACGCAAGATCCCGAGGTGAAATCTCCGCAAAATTC
AAGTTGTTCTTCGGGTTAGGCCTACTAGGGTTTTTCTTGGTGTTCGTCGTTTTAGCTGTGGTCAAGAATAGGAGAATGAG
AAAGAACAACCCGAATTTATGGAAGCTTATAGGGTTTCAGAAGCTCGGTTTCAGAAGCGAACACATATTAGAATGTGTTA
AAGAGAACCATGTGATTGGGAAAGGCGGACGAGGGATTGTCTACAAAGGGGTAATGCCAAACGGAGAAGAAGTTGCAGTC
AAGAAGCTCTTAACCATAACCAAAGGATCATCTCATGACAACGGTTTAGCCGCAGAGATTCAGACATTAGGTAGAATCAG
ACACAGAAACATAGTGAGATTGCTCGCTTTTTGTTCAAACAAAGACGTGAATCTCCTTGTTTACGAGTATATGCCTAATG
GTAGCCTCGGAGAAGTCTTGCACGGGAAAGCTGGAGTGTTTTTGAAATGGGAAACACGGTTGCAAATAGCGTTGGAAGCG
GCTAAGGGGTTGTGTTATCTTCACCATGATTGCTCGCCACTTATAATCCACCGTGATGTGAAGTCAAACAACATCTTGTT
GGGTCCTGAGTTTGAAGCTCATGTTGCTGATTTTGGGCTTGCTAAGTTTATGATGCAAGACAATGGAGCTTCCGAGTGCA
TGTCCTCGATCGCTGGCTCGTACGGCTACATCGCTCCAGAATATGCATATACACTGAGAATAGACGAGAAGAGCGATGTG
TACAGCTTCGGAGTAGTGTTATTGGAGCTGATTACGGGTCGAAAACCAGTAGATAATTTTGGGGAAGAAGGGATAGACAT
TGTGCAATGGTCAAAGATCCAAACAAACTGTAACAGACAAGGTGTGGTGAAGATCATTGACCAGAGATTGAGCAATATTC
CATTAGCAGAGGCCATGGAACTGTTCTTTGTGGCAATGCTATGTGTGCAAGAACATAGTGTTGAGAGACCGACCATGAGA
GAGGTTGTCCAGATGATCTCTCAGGCTAAACAGCCTAATACTTTCTAA
>12603755_protein_ID_12603757
MADKIFTFFLILSSISPLLCSSLISPLNLSLIRQANVLISLKQSFDSYDPSLDSWNIPNFNSLCSWTGVSCDNLNQSITR
LDLSNLNISGTISPEISRLSPSLVFLDISSNSFSGELPKEIYELSGLEVLNISSNVFEGELETRGFSQMTQLVTLDAYDN
SFNGSLPLSLTTLTRLEHLDLGGNYFDGEIPRSYGSFLSLKFLSLSGNDLRGRIPNELANITTLVQLYLGYYNDYRGGIP
ADFGRLINLVHLDLANCSLKGSIPAELGNLKNLEVLFLQTNELTGSVPRELGNMTSLKTLDLSNNFLEGEIPLELSGLQK
LQLFNLFFNRLHGEIPEFVSELPDLQILKLWHNNFTGKIPSKLGSNGNLIEIDLSTNKLTGLIPESLCFGRRLKILILFN
NFLFGPLPEDLGQCEPLWRFRLGQNFLTSKLPKGLIYLPNLSLLELQNNFLTGEIPEEEAGNAQFSSLTQINLSNNRLSG
PIPGSIRNLRSLQILLLGANRLSGQIPGEIGSLKSLLKIDMSRNNFSGKFPPEFGDCMSLTYLDLSHNQISGQIPVQISQ
IRILNYLNVSWNSFNQSLPNELGYMKSLTSADFSHNNFSGSVPTSGQFSYFNNTSFLGNPFLCGFSSNPCNGSQNQSQSQ
LLNQNNARSRGEISAKFKLFFGLGLLGFFLVFVVLAVVKNRRMRKNNPNLWKLIGFQKLGFRSEHILECVKENHVIGKGG
RGIVYKGVMPNGEEVAVKKLLTITKGSSHDNGLAAEIQTLGRIRHRNIVRLLAFCSNKDVNLLVYEYMPNGSLGEVLHGK
AGVFLKWETRLQIALEAAKGLCYLHHDCSPLIIHRDVKSNNILLGPEFEAHVADFGLAKFMMQDNGASECMSSIAGSYGY
IAPEYAYTLRIDEKSDVYSFGVVLLELITGRKPVDNFGEEGIDIVQWSKIQTNCNRQGVVKIIDQRLSNIPLAEAMELFF
VAMLCVQEHSVERPTMREVVQMISQAKQPNTF*
>12640578_construct_ID_YP0263
GTCCCATCACCAAACATTAAGTAGCACTCTTTTTCCTCTCTATATCTCTCACTCACACTTTTTCTCTATATCTTCTCCTC
AACTTGGATATGGGTGAAGCCGTAGAGGTCATGTTCGGAAATGGGTTCCCGGAGATTCACAAAGCCACATCACCCACTCA
AACCCTCCACTCTAACCAGCAAGACTGCCATTGGTATGAAGAAACCATCGATGATGATCTCAAGTGGTCTTTTGCCCTCA
ACAGTGTTCTCCATCAAGGAACTAGTGAGTACCAAGATATTGCTCTGTTGGACACCAAACGTTTTGGAAAGGTGCTTGTG
ATTGATGGGAAAATGCAAAGTGCTGAGAGAGATGAGTTTATCTACCATGAATGTTTGATCCATCCCGCTCTCCTTTTCCA
TCCCAACCCCAAGACTGTGTTTATAATGGGAGGAGGTGAAGGCTCTGCTGCAAGAGAAATACTAAAACACACGACGATCG
AGAAAGTTGTTATGTGTGATATTGATCAGGAAGTTGTTGATTTTTGCAGAAGATTTCTGACCGTTAACAGCGATGCTTTC
TGTAACAAAAAGCTTGAACTTGTGATCAAAGATGCAAAGGCTGAATTAGAGAAAAGGGAAGAGAAGTTTGATATCATAGT
GGGAGATTTAGCTGATCCAGTGGAAGGTGGACCTTGTTATCAGCTCTACACCAAATCCTTCTACCAAAACATTCTCAAAC
CCAAGCTTAGCCCTAATGGCATTTTTGTCACCCAGGCTGGACCAGCAGGAATATTCACTCATAAGGAAGTCTTCACATCA
ATCTACAACACCATGAAGCAAGTCTTCAAGTACGTGAAGGCTTACACAGCACATGTGCCATCATTTGCGGACACATGGGG
ATGGGTGATGGCATCGGACCACGAGTTTGACGTTGAAGTTGATGAAATGGATCGAAGAATCGAAGAGAGAGTTAACGGAG
AATTGATGTATCTAAACGCTCCTTCTTTCGTCTCTGCTGCTACTCTCAACAAAACCATCTCTCTCGCGCTAGAGAAGGAG
ACTGAAGTTTATAGTGAAGAGAATGCGAGATTCATTCATGGTCATGGTGTGGCGTACCGGCATATTTAAAGACGAACCGG
TTTCAGTTTCAGTGTTATTACCAAACCCATGTCACAAAAACAAAAGGCCGGTTTCTTTTCTCCGCACAGAACCGGGTGTT
GTCTTGAATCTTGATTACTTTGGTTCGGTTTTATTTTCTACATTGCTTTTTGTTTTCTTGTTCTTCCCTCAAGTTATTCC
GGTTTAACAAGACTATATTGCTTACTAA
>12640578_protein_ID_12640579
MGEAVEVMFGNGFPEIHKATSPTQTLHSNQQDCHWYEETIDDDLKWSFALNSVLHQGTSEYQDIALLDTKRFGKVLVIDG
KMQSAERDEFIYHECLIHPALLFHPNPKTVFIMGGGEGSAAREILKHTTIEKVVMCDIDQEVVDFCRRFLTVNSDAFCNK
KLELVIKDAKAELEKREEKFDIIVGDLADPVEGGPCYQLYTKSFYQNILKPKLSPNGIFVTQAGPAGIFTHKEVFTSIYN
TMKQVFKYVKAYTAHVPSFADTWGWVMASDHEFDVEVDEMDRRIEERVNGELMYLNAPSFVSAATLNKTISLALEKETEV
YSEENARFIHGHGVAYRHI*
>12647555_construct_ID_YP0018
ATCTCACATCACAATTCACATCTCCTCGAACAAACAAATTATAAACCCATTTTCCTTCATAAATTTCTAAAATAAAACCC
CTTAAACTTTCATTCACATCATCCAACCCCCAATGGGTCGAATCTTGAACCGTACCGTGTTAATGACTCTTCTAGTCGTA
ACAATGGCCGGAACAGCATTCTCCGGTAGCTTCAACGAAGAGTTTGACTTAACTTGGGGTGAACACAGAGGCAAAATCTT
CAGTGGAGGAAAAATGTTGTCACTCTCACTAGACCGGGTTTCCGGGTCGGGTTTTAAATCCAAGAAAGAATATTTGTTCG
GAAGAATCGACATGCAGCTTAAACTCGTCGCCGGTAACTCCGCTGGAACCGTCACTGCCTACTACTTGTCATCGGAAGGA
CCAACACACGACGAGATAGACTTTGAGTTTCTTGGTAATGAAACAGGGAAGCCTTATGTTCTTCACACTAATGTATTTGC
TCAAGGCAAAGGAAACAGAGAACAACAGTTTTATCTCTGGTTTGATCCAACCAAGAACTTCCACACTTATTCTCTTGTCT
GGAGACCACAACACATCATATTTATGGTAGATAATGTTCCAATCAGAGTATTCAACAATGCAGAGCAACTTGGTGTTCCA
TTTCCCAAGAACCAACCAATGAAGATATACTCGAGTTTATGGAATGCAGATGATTGGGCTACAAGAGGTGGTTTGGTTAA
GACAGATTGGTCTAAAGCTCCTTTCACAGCTTACTACAGAGGCTTTAACGCTGCAGCTTGTACTGTTTCTTCAGGGTCAT
CTTTCTGTGATCCTAAGTTTAAGAGTTCTTTTACTAATGGTGAATCTCAAGTGGCTAATGAGCTTAATGCTTATGGGAGA
AGAAGATTAAGATGGGTTCAGAAGTATTTTATGATTTATGATTATTGTTCTGATTTAAAAAGGTTTCCTCPAGGATTCCC
ACCAGAGTGTAGGAAGTCTAGAGTCTAAAAACCAATGATTCTCTCTTTGTTGTTGTTTAGTGCAAATTAAATTCTCTTTG
TTGTTTCTTTAATAAATTGATTTGATTTTTCTTC
>12647555_protein_ID_12647556
MGRILNRTVLMTLLVVTMAGTAFSGSFNEEFDLTWGEHRGKIFSGGKMLSLSLDRVSGSGFKSKKEYLFGRIDMQLKLVA
GNSAGTVTAYYLSSEGPTHDEIDFEFLGNETGKPYVLHTNVFAQGKGNREQQFYLWFDPTKNFHTYSLVWRPQHIIFMVD
NVPIRVFNNAEQLGVPFPKNQPMKIYSSLWNADDWATRGGLVKTDWSKAPFTAYYRGFNAAACTVSSGSSFCDPKFKSSF
TNGESQVANELNAYGRRRLRWVQKYFMIYDYCSDLKRFPQGFPPECRKSRV*
>12649228_construct_ID_YP0003
GCTCCTTTCTCGTCTCTGTCTTCTTCGTCCTCATTCGTTTTAAAGCATCAAAATTTCATCAACCCAAAATAGATTAAAAA
AATCTGTAGCTTTCGCATGTAAATCTCTCTTTGAAGGTTCCTAACTCGTTAATCGTAACTCACAGTGACTCGTTCGAGTC
AAAGTCTCTGTCTTTAGCTCAAACCATGGCTAGTAACAACCCTCACGACAACCTTTCTGACCAAACTCCTTCTGATGATT
TCTTCGAGCAAATCCTCGGCCTTCCTAACTTCTCAGCCTCTTCTGCCGCCGGTTTATCTGGAGTTGACGGAGGATTAGGT
GGTGGAGCACCGCCTATGATGCTGCAGTTGGGTTCCGGAGAAGAAGGAAGTCACATGGGTGGCTTAGGAGGAAGTGGACC
AACTGGGTTTCACAATCAGATGTTTCCTTTGGGGTTAAGTCTTGATCAAGGGAAAGGACCTGGGTTTCTTAGACCTGAAG
GAGGACATGGAAGTGGGAAAAGATTCTCAGATGATGTTGTTGATAATCGATGTTCTTCTATGAAACCTGTTTTCCACGGG
CAGCCTATGCAACAGCCACCTCCATCGGCCCCACATCAGCCTACTTCAATCCGTCCCAGGGTTCGAGCTAGGCGTGGTCA
GGCTACTGATCCACATAGCATCGCTGAGCGGCTACGTAGAGAAAGAATAGCAGAACGGATCAGGGCGCTGCAGGPACTTG
TACCTACTGTGAACAAGACCGATAGAGCTGCTATGATCGATGAGATTGTCGATTATGTAAAGTTTCTCAGGCTCCAAGTC
AAGGTTTTGAGCATGAGCCGACTTGGTGGAGCCGGTGCGGTTGCTCCACTTGTTACTGATATGCCTCTTTCATCATCAGT
TGAGGATGAAACGGGTGAGGGTGGAAGGACTCCGCAACCAGCGTGGGAGAAATGGTCTAACGATGGGACTGAACGTCAAG
TGGCTAAACTGATGGAAGAGAACGTTGGAGCCGCGATGCAGCTTCTTCAATCAAAGGCTCTTTGTATGATGCCAATCTCA
TTGGCAATGGCAATTTACCATTCTCAACCTCCGGATACATCTTCAGTGGTCAAGCCTGAGAACAATCCTCCACAGTAGGA
TTTCTGCAATAAAGAGTTTGTACAGCTAATCCAACTGTCCAACATGGGTTTTTCTTCTGCTCTAATGACTCTGGTTTCTT
CTCTCCTCTCTCACCCACTTGAAAGGTAAAAAAGTGAAAAAGGCTTTGTAGATGGAATCAATGTAGGATTTGCAGTAGAG
GGAAAAAAAATGTCAAAAAGCTCAATTGATCAAGTATTATTGTAATCATTGTACCTTTATTTTAGGTGGACTTTGATGAA
AGCAACTTTTTGTTTTCAAGACTTTAGTGGGAGGTTGAGGAAGGAGCTTGAAGGGTGTTATTTATTAGTAGTAGTAGTAG
TGGGAAGTTGTGGGACCTTGTTGAGTTGTGTTCAAATTGAAGAAAAAACAAGTATTTGTAATTTGTCACCCCTTGTATTA
TTATTTATTTTGTATGA
>12649228_protein_ID_12649229
MASNNPHDNLSDQTPSDDFFEQILGLPNFSASSAAGLSGVDGGLGGGAPPMNLQLGSGEEGSHMGGLGGSGPTGFHNQMF
PLGLSLDQGKGPGFLRPEGGHGSGKRFSDDVVDNRCSSMKPVFHGQPMQQPPPSAPHQPTSIRPRVRARRGQATDPHSIA
ERLRRERIAERIRALQELVPTVNKTDRAANIDEIVDYVKFLRLQVKVLSMSRLGGAGAVAPLVTDMPLSSSVEDETGEGG
RTPQPAWEKWSNDGTERQVAKLMEENVGAAMQLLQSKALCMMPISLANAIYHSQPPDTSSVVKPENNPPQ*
>12658070_construct_ID_YP0271
CACACTTAAAGCTTTCGTCTTTACCTCTTCCCTTCTCTCTCTCTATCTAAAAAGAGTTCCGAGAAGAAGATCATCATCAA
TGGCGACTTCTCTCTTCTTCATGTCAACAGATCAAAACTCCGTCGGAAACCCAAACGATCTTCTGAGAAACACCCGTCTT
GTCGTCAACAGCTCCGGCGAGATCCGGACAGAGACACTGAAGAGTCGTGGTCGGAAACCAGGATCGAAGACAGGTCAGCA
AAAACAGAAGAAACCAACGTTGAGAGGAATGGGTGTAGCAAAGCTCGAGCGTCAGAGAATCGAAGAAGAAAAGAAGCAAC
TCGCCGCCGCCACAGTCGGAGACACGTCATCAGTAGCATCGATCTCTAACAACGCTACCCGTTTACCCGTACCGGTAGAC
CCGGGTGTTGTGCTACAAGGCTTCCCAAGCTCACTCGGGAGCAACAGGATCTATTGTGGTGGAGTCGGGTCGGGTCAGGT
TATGATCGACCCGGTTATTTCTCCATGGGGTTTTGTTGAGACCTCCTCCACTACTCATGAGCTCTCTTCAATCTCAAATC
CTCAAATGTTTAACGCTTCTTCCAATAATCGCTGTGACACTTGCTTCAAGAAGAAACGTTTGGATGGTGATCAGAATAAT
GTAGTTCGATCCAACGGTGGTGGATTTTCGAAATACACAATGATTCCTCCTCCGATGAACGGCTACGATCAGTATCTTCT
TCAATCAGATCATCATCAGAGGAGCCAAGGTTTCCTTTATGATCATAGAATCGCTAGAGCAGCTTCAGTTTCTGCTTCTA
GTACTACTATTAATCCTTATTTCAACGAGGCAACAAATCATACGGGACCAATGGAGGAATTTGGGAGCTACATGGAAGGA
AACCCTAGAAATGGATCAGGAGGTGTGAAGGAGTACGAGTTTTTTCCGGGGAAATATGGTGAAAGAGTTTCAGTGGTGGC
TAAAACGTCGTCACTCGTAGGTGATTGCAGTCCTAATACCATTGATTTGTCCTTGAAGCTTTAAATGTTTTATCTTTCTA
TATTGATTTAAACAAAATCGTCTCTTTAAAGAAAAAACATTTTAAGTAGATGAAAGTAAGAAACAGAAGAAAAAAAAGAG
AGAGCCTTTTTTGGTGTATGCATCTGAGAGCTGAGTCGAAAGAAAGATTCAGCTTTTGGATTACCCTTTTGGTTGTTTAT
TATGAGATTCTAACCTAAACACTCAGACATATATGTTCTGTTCTCTTCCTTAATTGTTGTCATGAAACTTCTC
>12658070_protein_ID_12658072
MATSLFFMSTDQNSVGNPNDLLRNTRLVVNSSGEIRTETLKSRGRKPGSKTGQQKQKKPTLRGMGVAKLERQRIEEEKKQ
LAAATVGDTSSVASISNNATRLPVPVDPGVVLQGFPSSLGSNRIYCGGVGSGQVMIDPVISPWGFVETSSTTHELSSISN
PQMFNASSNNRCDTCFKKKRLDGDQNNVVRSNGGGFSKYTMIPPPMNGYDQYLLQSDHHQRSQGFLYDHRIARAASVSAS
STTINPYFNEATNHTGPMEEFGSYMEGNPRNGSGGVKEYEFFPGKYGERVSVVAKTSSLVGDCSPNTIDLSLKL*
>12676237_construct_ID_YP0230
CGAAGGCACGACAAGCATCAATCCGCCTCAAGCAGTAGCAGCAGGAAACGTAGCAGGGAACATGGCAGGAGCTCATGGAA
TGGGCAGTAGATCGATGCCAAGACCAATGGTTGCACATAACATGCAGAGGATGCAGCAATCTCAAGGCATGATGGCTTAT
ATTTCCCGGCACAGGCAGGGCTTAACCCGAGTGTTCCGCTGCAGCAGCAGCGCGGGATGGCTCAAACCGCACCAGCAGCA
ACAGCTAAGAAGGAAAGATCCCGGAATGGGTATGTCAGGTTACGCACCTCCTAACAAATCCAGACGCCTCTAAAGGTAAA
ATCGAGATCATCAGTCTCGGGTTAGAATCTGTGTGTTTGCCGCAGAAGAAAGCGTTGCGATTTGCTTTATAGAGTAGAGT
TAGATTGTAATGCAGCATGTGGAATGTTGCTATTCATATGGATGGATTGGATTCTCTGTAGTTTTTGTATAAACATCCTC
TCAAGTATTTGTTAATTATATTAGATCATCATTTCTCTT
>12676237_protein_ID_12676238
EGTTSINPPQAVAAGNVAGNMAGAHGMGSRSMPRPMVAHNMQRMQQSQGMMAYNFPAQAGLNPSVPLQQQRGMAQPHQQQ
QLRRKDPGMGMSGYAPPNKSRRL*
>12721583_construct_ID_YP0071
ATGGCGATGAGACTTTTGAAGACTCATCTTCTGTTTCTGCATCTGTATCTATTTTTCTCACCATGTTTCGCTTACACTGA
CATGGAAGTTCTTCTCAATCTCAAATCCTCCATGATTGGTCCTAAAGGACACGGTCTCCACGACTGGATTCACTCATCTT
CTCCGGATGCTCACTGTTCTTTCTCCGGCGTCTCATGTGACGACGATGCTCGTGTTATCTCTCTCAACGTCTCCTTCACT
CCTTTGTTTGGTACAATCTCACCAGAGATTGGGATGTTGACTCATTTGGTGAATCTAACTTTAGCTGCCAACAACTTCAC
CGGTGAATTACCATTGGAGATGAAGAGTCTAACTTCTCTCAAGGTTTTGAATATCTCCAACAATGGTAACCTTACTGGAA
CATTCCCTGGAGAGATTTTAAAAGCTATGGTTGATCTTGAAGTTCTTGACACTTATAACAACAATTTCAACGGTAAGTTA
CCACCGGAGATGTCAGAGCTTAAGAAGCTTAAATACCTCTCTTTCGGTGGAAATTTCTTCAGCGGAGAGATTCCAGAGAG
TTATGGAGATATTCAAAGCTTAGAGTATCTTGGTCTCAACGGAGCTGGACTCTCCGGTAAATCTCCGGCGTTTCTTTCCC
GCCTCAAGAACTTAAGAGAAATGTATATTGGCTACTACAACAGCTACACCGGTGGTGTTCCACCGGAGTTCGGTGGTTTA
ACAAAGCTTGAGATCCTCGACATGGCGAGCTGTACACTCACCGGAGAGATTCCGACGAGTTTAAGTAACCTGAAACATCT
ACATACTCTGTTTCTTCACATCAACAACTTAACCGGTCATATACCACCGGAGCTTTCCGGTTTAGTCAGCTTGAAATCTC
TCGATTTATCAATCAATCAGTTAACCGGAGAAATCCCTCAAAGCTTCATCAATCTCGGAAACATTACTCTAATCAATCTC
TTCAGAAACAATCTCTACGGACAAATACCAGAGGCCATCGGAGAATTACCAAAACTCGAAGTCTTCGAAGTATGGGAGAA
CAATTTCACGTTACAATTACCGGCGAATCTTGGCCGGAACGGGAATCTAATAAAGCTTGATGTCTCTGATAATCATCTCA
CCGGACTTATCCCCAAGGACTTATGCAGAGGTGAGAAATTAGAGATGTTAATTCTCTCTAACAACTTCTTCTTTGGTCCA
ATTCCAGAAGAGCTTGGTAAATGCAAATCCTTAACCAAAATCAGAATCGTTAAGAATCTTCTCAACGGCACTGTTCCGGC
GGGGCTTTTCAATCTACCGTTAGTTACGATTATCGAACTCACTGATAATTTCTTCTCCGGTGAACTTCCGGTAACGATGT
CCGGCGATGTTCTCGATCAGATTTACCTCTCTAACAACTGGTTTTCCGGCGAGATTCCACCTGCGATTGGTAATTTCCCC
AATCTACAGACTCTATTCTTAGATCGGAACCGATTTCGCGGCAACATTCCGAGAGAAATCTTCGAATTGAAGCATTTATC
GAGGATCAACACAAGTGCGAACAACATCACCGGCGGTATTCCAGATTCAATCTCTCGCTGCTCAACTTTAATCTCCGTCG
ATCTCAGCCGTAACCGAATCAACGGAGAAATCCCTAAAGGGATCAACAACGTGAAAAACTTAGGAACTCTAAATATCTCC
GGTAATCAATTAACCGGTTCAATCCCTACCGGAATCGGAAACATGACGAGTTTAACAACTCTCGATCTCTCTTTCAACGA
TCTCTCCGGTAGAGTACCACTCGGTGGTCAATTCTTGGTGTTCAACGAAACTTCCTTCGCCGGAAACACTTACCTCTGTC
TCCCTCACCGTGTCTCTTGTCCAACACGGCCAGGACAAACCTCCGATCACAATCACACGGCGTTGTTCTCACCGTCAAGG
ATCGTAATCACGGTTATCGCAGCGATCACCGGTTTGATCCTAATCAGTGTAGCGATTCGTCAGATGAATAAGAAGAAGAA
CCAGAAATCTCTCGCCTGGAAACTAACCGCCTTCCAGAAACTAGATTTCAAATCTGAAGACGTTCTCGAGTGTCTTAAAG
AAGAGAACATAATCGGTAAAGGCGGAGCTGGAATTGTCTACCGTGGATCAATGCCAAACAACGTAGACGTCGCGATTAAA
CGACTCGTTGGCCGTGGGACCGGGAGGAGCGATCATGGATTCACGGCGGAGATTCAAACTTTGGGGAGAATCCGCCACCG
TCACATAGTGAGACTTCTTGGTTACGTAGCGAACAAGGATACGAATCTCCTTCTTTATGAGTACATGCCTAATGGAAGCC
TTGGAGAGCTTTTGCATGGATCTAAAGGTGGTCATCTTCAATGGGAGACGAGACATAGAGTAGCCGTGGAAGCTGCAAAG
GGCTTGTGTTATCTTCACCATGATTGTTCACCATTGATCTTGCATAGAGATGTTAAGTCCAATAACATTCTTTTGGACTC
TGATTTTGAAGCCCATGTTGCTGATTTTGGGCTTGCTAAGTTCTTAGTTGATGGTGCTGCTTCTGAGTGTATGTCTTCAA
TTGCTGGCTCTTATGGATACATCGCCCCAGAGTATGCATATACCTTGAAAGTGGACGAGAAGAGTGATGTGTATAGTTTC
GGAGTGGTTTTGTTGGAGTTAATAGCTGGGAAGAAACCTGTTGGTGAATTTGGAGAAGGAGTGGATATAGTTAGGTGGGT
GAGGAACACGGAAGAGGAGATAACTCAGCCATCGGATGCTGCTATTGTTGTTGCGATTGTTGACCCGAGGTTGACTGGTT
ACCCGTTGACAAGTGTGATTCATGTGTTCAAGATCGCAATGATGTGTGTGGAGGAAGAAGCCGCGGCAAGGCCTACGATG
AGGGAAGTTGTGCACATGCTCACTAACCCTCCTAAATCCGTGGCGAACTTGATCGCGTTCTGA
>12721583_protein_ID_12721584
MAMRLLKTHLLFLHLYLFFSPCFAYTDMEVLLNLKSSMIGPKGHGLHDWIHSSSPDAHCSFSGVSCDDDARVISLNVSFT
PLFGTISPEIGMLTHLVNLTLAANNFTGELPLEMKSLTSLKVLNISNNGNLTGTFPGEILKAMVDLEVLDTYNNNFNGKL
PPEMSELKKLKYLSFGGNFFSGEIPESYGDIQSLEYLGLNGAGLSGKSPAFLSRLKNLREMYIGYYNSYTGGVPPEFGGL
TKLEILDMASCTLTGEIPTSLSNLKHLHTLFLHINNLTGHIPPELSGLVSLKSLDLSINQLTGEIPQSFINLGNITLINL
FRNNLYGQIPEAIGELPKLEVFEVWENNFTLQLPANLGRNGNLIKLDVSDNHLTGLIPKDLCRGEKLEMLILSNNFFFGP
IPEELGKCKSLTKIRIVKNLLNGTVPAGLFNLPLVTIIELTDNFFSGELPVTMSGDVLDQIYLSNNWFSGEIPPAIGNFP
NLQTLFLDRNRFRGNIPREIFELKHLSRINTSANNITGGIPDSISRCSTLISVDLSRNRINGEIPKGINNVKNLGTLNIS
GNQLTGSIPTGIGNMTSLTTLDLSFNDLSGRVPLGGQFLVFNETSFAGNTYLCLPHRVSCPTRPGQTSDHNHTALFSPSR
IVITVIAAITGLILISVAIRQMNKKKNQKSLAWKLTAFQKLDFKSEDVLECLKEENIIGKGGAGIVYRGSMPNNVDVAIK
RLVGRGTGRSDHGFTAEIQTLGRIRHRHIVRLLGYVANKDTNLLLYEYMPNGSLGELLHGSKGGHLQWETRHRVAVEAAK
GLCYLHHDCSPLILHRDVKSNNILLDSDFEAHVADFGLAKFLVDGAASECMSSIAGSYGYIAPEYAYTLKVDEKSDVYSF
GVVLLELIAGKKPVGEFGEGVDIVRWVRNTEEEITQPSDAAIVVAIVDPRLTGYPLTSVIHVFKIAMMCVEEEAAARPTM
REVVHMLTNPPKSVANLIAF*
>13593439_construct_ID_YP0122
AAGCCACACAATCTCTTTTCTTCTCTCTCTCTCTGTTATATCTCTTCTGTTTAATTCTTTTATTCTTCTTCGTCTATCTT
CTCCTATAATCTCTTCTCTCTCCCTCTTCACCTAAAGAATAAGAAGAAAAATAATTCACATCTTTATGCAAACTACTTTC
TTGTAGGGTTTTAGGAGCTATCTCTATTGTCTTGGTTCTGATACAAAGTTTTGTAATTTTCATGGTATGAGPAGATTTGC
CTTTCTATTTTGTTTATTGGTTCTTTTTAACTTTTTCTTGGAGATGGGTTCTTGTAGATCTTAATGAAACTTCTGTTTTT
GTCCCAAAAAGAGTTTTCTTTTTTCTTCTCTTCTTTTTGGGTTTTCAATTCTTGAGAGACATGGCAAGAGATCAGTTCTA
TGGTCACAATAACCATCATCATCAAGAGCAACAACATCAAATGATTAATCAGATCCAAGGGTTTGATGAGACAAACCAAA
ACCCAACCGATCATCATCATTACAATCATCAGATCTTTGGCTCAAACTCCAACATGGGTATGATGATAGACTTCTCTAAG
CAACAACAGATTAGGATGACAAGTGGTTCGGATCATCATCATCATCATCATCAGACAAGTGGTGGTACTGATCAGAATCA
GCTTCTGGAAGATTCTTCATCTGCCATGAGACTATGCAATGTTAATAATGATTTCCCAAGTGAAGTAAATGATGAGAGAC
CACCACAAAGACCAAGCCAAGGTCTTTCCCTTTCTCTCTCCTCTTCAAATCCTACAAGCATCAGTCTCCAATCTTTCGAA
CTCAGACCCCAACAACAACAACAACAAGGGTATTCCGGTAATAAATCAACACAACATCAGAATCTCCAACACACGCAGAT
GATGATGATGATGATGAATAGTCACCACCAAAACAACAACAATAACAATCATCAGCATCATAATCATCATCAGTTTCAGA
TTGGGAGTTCCAAGTATTTGAGTCCAGCTCAAGAGCTACTGAGTGAGTTTTGCAGTCTTGGAGTAAAGGAAAGCGATGAA
GAAGTGATGATGATGAAGCATAAGAAGAAGCAAAAGGGTAAACAACAAGAAGAGTGGGACACAAGTCACCACAGCAACAA
TGATCAACATGACCAATCTGCGACTACTTCTTCAAAGAAACATGTTCCACCACTTCACTCTCTTGAGTTCATGGAACTTC
AGAAAAGAAAAGCCAAGTTGCTCTCCATGCTCGAAGAGCTTAAAAGAAGATATGGACATTACCGAGAGCAAATGAGAGTT
GCGGCGGCAGCCTTTGAAGCGGCGGTTGGACTAGGAGGGGCAGAGATATACACTGCGTTAGCGTCAAGGGCAATGTCAAG
ACACTTTCGGTGTTTAAAAGACGGACTTGTGGGACAGATTCAAGCAACAAGTCAAGCTTTGGGAGAGAGAGAAGAGGATA
ATCGTGCGGTTTCTATTGCAGCACGTGGAGAAACTCCACGGTTGAGATTGCTCGATCAAGCTTTGCGGCAACAGAAATCG
TATCGCCAAATGACTCTTGTTGACGCTCATCCTTGGCGTCCACAACGCGGCTTGCCTGAACGCGCAGTCACAACGTTGAG
AGCTTGGCTCTTTGAACACTTTCTTCACCCATATCCGAGCGATGTTGATAAGCATATATTGGCCCGACAAACTGGTTTAT
CAAGAAGTCAGGTATCAAATTGGTTTATTAATGCAAGAGTTAGGCTATGGAAACCAATGATTGAAGAAATGTACTGTGAA
GAAACAAGAAGTGAACAAATGGAGATTACAAACCCGATGATGATCGATACTAAACCGGACCCGGACCAGTTGATCCGTGT
CGAACCGGAATCTTTATCCTCAATAGTGACAAACCCTACATCCAAATCCGGTCACAACTCAACCCATGGAACGATGTCGT
TAGGGTCAACGTTTGACTTTTCCTTGTACGGTAACCAAGCTGTGACATACGCTGGTGAAGGAGGGCCACGTGGTGACGTT
TCCTTGACGCTTGGGTTACAACGTAACGATGGTAACGGTGGTGTGAGTTTAGCGTTGTCTCCAGTGACGGCTCAAGGTGG
CCAACTTTTCTACGGTAGAGACCACATTGAAGAAGGACCGGTTCAATATTCAGCGTCGATGTTAGATGATGATCAAGTTC
AGAATTTGCCTTATAGGAATTTGATGGGAGCTCAATTACTTCATGATATTGTTTGAGATTAAAAGATTAGGACCAAAGTT
ATCGATACATATTTTCCAAAACCGATTCGGTTATGTAACGGTTTAGTTAGATAAAAACCAAATTAGATATTTATATATAC
CGTTGTCTGATTGGATTGGAGGATTGGTGGACAAGGAGATATTATTAATGTATGAGTTAGTTGGTTCGTCAATATCACTT
GTAGGATATTTTCATTTTGTTTTTTAAAATATATTATTGAGAGGTTTTTTTCTC
>13593439_protein_ID_13593440
MARDQFYGHNNHHHQEQQHQMINQIQGFDETNQNPTDHHHYNHQIFGSNSNMGMMIDFSKQQQIRMTSGSDHHHHHHQTS
GGTDQNQLLEDSSSAMRLCNVNNDFPSEVNDERPPQRPSQGLSLSLSSSNPTSISLQSFELRPQQQQQQGYSGNKSTQHQ
NLQHTQMMMMMMNSHHQNNNNNNHQHHNHHQFQIGSSKYLSPAQELLSEFCSLGVKESDEEVMMNKHKKKQKGKQQEEWD
TSHHSNNDQHDQSATTSSKKHVPPLHSLEFMELQKRKAKLLSMLEELKRRYGHYREQMRVAAAAFEAAVGLGGAEIYTAL
ASRANSRHFRCLKDGLVGQIQATSQALGEREEDNRAVSIAARGETPRLRLLDQALRQQKSYRQMTLVDAHPWRPQRGLPE
RAVTTLRAWLFEHFLHPYPSDVDKHILARQTGLSRSQVSNWFINARVRLWKPMIEEMYCEETRSEQMEITNPMMIDTKPD
PDQLIRVEPESLSSIVTNPTSKSGHNSTHGTMSLGSTFDFSLYGNQAVTYAGEGGPRGDVSLTLGLQRNDGNGGVSLALS
PVTAQGGQLFYGRDHIEEGPVQYSASMLDDDQVQNLPYRNLMGAQLLHDIV*
>13612380_construct_ID_YP0015
AAAAAAGTTCAGATATTTGATAAATCAATCAACAAAACAAAAAAAACTCTATAGTTAGTTTCTCTGAAAATGTACGGACA
GTGCAATATAGAATCCGACTACGCTTTGTTGGAGTCGATAACACGTCACTTGCTAGGAGGAGGAGGAGAGAACGAGCTGC
GACTCAATGAGTCAACACCGAGTTCGTGTTTCACAGAGAGTTGGGGAGGTTTGCCATTGAAAGAGAATGATTCAGAGGAC
ATGTTGGTGTACGGACTCCTCAAAGATGCCTTCCATTTTGACACGTCATCATCGGACTTGAGCTGTCTTTTTGATTTTCC
GGCGGTTAAAGTCGAGCCAACTGAGAACTTTACGGCGATGGAGGAGAAACCAAAGAAAGCGATACCGGTTACGGAGACGG
CAGTGAAGGCGAAGCATTACAGAGGAGTGAGGCAGAGACCGTGGGGGAAATTCGCGGCGGAGATACGTGATCCGGCGAAG
AATGGAGCTAGGGTTTGGTTAGGGACGTTTGAGACGGCGGAAGATGCGGCTTTAGCTTACGATATAGCTGCTTTTAGGAT
GCGTGGTTCCCGCGCTTTATTGAATTTTCCGTTGAGGGTTAATTCCGGTGAACCTGACCCGGTTCGGATCACGTCTAAGA
GATCTTCTTCGTCGTCGTCGTCGTCGTCCTCTTCTACGTCGTCGTCGTAAAACGGGAAGTTGAAACGAAGGAGAAAAGCA
GAGAATCTGACGTCGGAGGTGGTGCAGGTGAAGTGTGAGGTTGGTGATGAGACACGTGTTGATGAGTTATTGGTTTCATA
AGTTTGATCTTGTGTGTTTTGTAGTTGAATAGTTTTGCTATA~ATGTTGAGGCACCAAGTAAAAGTGTTCCCGTGATGTA
AATTAGTTACTAAACAGAGCCATATATCTTCAATCCATAACAAAATAGACACACTTTAATAAAGCCGTGAGTGTTATTTT
TC
>13612380_protein_ID_13612381
MYGQCNIESDYALLESITRHLLGGGGENELRLNESTPSSCFTESWGGLPLKENDSEDMLVYGLLKDAFHFDTSSSDLSCL
FDFPAVKVEPTENFTANEEKPKKAIPVTETAVKAKHYRGVRQRPWGKFAAEIRDPAKNGARVWLGTFETAEDAALAYDIA
AFRMRGSRALLNFPLRVNSGEPDPVRITSKRSSSSSSSSSSSTSSSENGKLKRRRKAENLTSEVVQVKCEVGDETRVDEL
LVS*

TABLE 2
Promoter Expression Report # 1
Report Date: January 31, 2003; Revised August 15, 2003
Promoter Tested In: Arabidopsis thaliana, WS ecotype
Spatial expression summary:

| | |
|---|---|
| Flower | (M)upper part of receptacle, (M)base of ovary |
| Flower | (M)pedicel, (M)receptacle, silique, (M)carpel |
| Stem | (H)cortex, (H)pith |
| Hypocotyl | (M)cortex |
| Primary Root | (H)vascular, (M)cap |

Observed expression pattern: T1 mature: Expression was specific to the top of the receptacle and
base of gynoecium of immature flowers. Not detected in any other organs. T2 seedlings: No expression
observed. T2 mature: In addition to the original expression observed in T1 mature plants, expression is
observed in pith cells near the apex of the inflorescence meristem and stem-pedicel junctions. T3
seedling: Expressed at cotyledon-hypocotyl junction, root vascular, and root tip epidermis. This
expression is similar to the original 2-component line CS9107.
Expected expression pattern: The candidate was selected from a 2-component line with multiple
inserts. The target expression pattern was lateral root cap and older vascular cells, especially in
hypocotyls.
Selection Criteria: Arabidopsis 2-component line CS9107 (J1911) was selected to test promoter
reconstitution and validation. T-DNA flanking sequences were isolated by TAIL-PCR and the fragment
cloned into pNewBin4-HAP1-GFP vector to validate expression.
Gene: 2 kb seq. is in 7 kb repeat region on Chr.2 where no genes are annotated.
GenBank: NM_127894Arabidopsis thaliana leucine-rich repeat transmembrane protein kinase,
putative (At2g23300) mRNA, complete cds gi|18400232|ref|NM_127894.1|[18400232]
Source Promoter Organism: Arabidopsis thaliana WS

| | |
|---|---|
| Vector: | pNewBin4-HAP1-GFP |
| Marker Type: | X GFP-ER |
| Generation Screened: | X T1 Mature X T2 Seedling X T2 Mature X T3 Seedling |

Bidirectionality: NO Exons: NO Repeats: none noted
Promoter utility
Trait-Subtrait Area: Among other uses this promoter sequence could be useful to improve:

PG&D- abscission, plant size
Nutrients- nitrogen utilization

Utility: Promoter may be useful in fruit abscission but as it appears the expression overlaps the base of
the gynoecium, it may be useful to overexpress genes thought to be important in supplying nutrients to
the gynoecium or genes important in development of carpel primordia.

| | |
|---|---|
| Construct: | YP0001 |
| Promoter Candidate I.D: | 13148168 (Old ID: CS9107-1) |
| cDNA I.D: | 12736079 |
| T1 lines expressing (T2 seed): | SR00375-01, -02, -03, -04, -05 |

Promoter Expression Report # 2
Report Date: January 31, 2003
Promoter Tested In: Arabidopsis thaliana, WS ecotype
Spatial expression summary:

| | |
|---|---|
| Ovule | Pre-fertilization: (H)inner integument |
| | Post-fertilization: (M)seed coat, (M)endothelium |
| Root | (H)epidermis, (H)atrichoblast |
| Cotyledons | (L)epidermis |

Observed expression pattern: T1 mature: GFP expression exists in the inner integument of
ovules. T2 seedling: Expression exists in root epidermal atrichoblast cells. T2 mature: Same
expression exists as T1 mature. T3 seedlings: Same expression, plus additional weak epidermal
expression was observed in cotyledons.
Expected expression pattern: flower buds, ovules, mature flower, and silique

| | |
|---|---|
| Selection Criteria: | Arabidopsis 2-component line CS9180(J2592). |
| Gene: | water channel-like protein″ major intrinsic protein (MIP) |
| | family |
| GenBank: | NM_118469Arabidopsis thaliana major intrinsic protein |
| | (MIP) family (At4g23400) mRNA, complete cds |
| | gi\|30686182\|ref\|NM_118469.2\|[30686182] |

Source Promoter Organism: Arabidopsis thaliana WS

| | |
|---|---|
| Vector: | pNewBin4-HAP1-GFP |
| Marker Type: | X GFP-ER |
| Generation Screened: | X T1 Mature X T2 Seedling X T2 Mature X T3 Seedling |

Bidirectionality: NO Exons: NO Repeats: None Noted
Promoter utility
Utility: Promoter could be used to misexpress any genes playing a role in seed size. It will also
have utility in misexpressing genes important in root hair initiation to try to get the plant to
generate more or fewer root hairs to enhance nutrient utilization and drought tolerance.

| | |
|---|---|
| Construct: | YP0007 |
| Promoter Candidate I.D: | 13148318 (Old ID: CS9180-3) |
| cDNA I.D: | 12703041 (Old I.D: 12332468) |
| T1 lines expressing (T2 seed): | SR00408-01, -02, -05 |

Promoter Expression Report # 3
Report Date: January 31, 2003
Promoter Tested In: Arabidopsis thaliana, WS ecotype
Spatial expression summary:

| | |
|---|---|
| Leaf | (L)vascular |
| Hypocotyl | (L)epidermis |
| Primary Root | (H)epidermis, (H)cap |
| Lateral root | (H)epidermis, (H)cap |

Observed expression pattern: T1 mature: Low GFP expression was detected throughout the
vasculature of leaves of mature plants. T2 seedling: No expression was detected in the
vasculature of seedlings. T2 mature: Transformation events which expressed as T1 plants were
screened as T2 plants and no expression was detected. This line was re-screened as T1 plants and
leaf expression was not detected in 3 independent events. T3 seedling: New expression was
observed in T3 seedlings which was not observed in T2 seedlings. Strong primary and lateral root
tip expression and weak hypocotyl epidermal expression exists.

| | |
|---|---|
| Expected expression pattern: | High in leaves. Low in tissues like roots or flowers |
| Selection Criteria: | Arabidopsis Public; Sauer N. EMBO J 1990 9: 3045-3050 |
| Gene: | Glucose transporter (Sugar carrier) STP1 |
| GenBank: | NM_100998Arabidopsis thaliana glucose |
| | transporter |
| | (At1g11260) mRNA, complete cds, |
| | gi\|30682126\|ref\|NM_100998.2\|[30682126] |
| Source Promoter Organism: | Arabidopsis thaliana WS |
| Vector: | pNewBin4-GFP Direct fusion construct |
| Marker Type: | X GFP-ER |
| Generation Screened: | X T1 Mature X T2 Seedling X T2 Mature XT3 Seedling |

Bidirectionality: NO Exons: NO Repeats: None Noted
Promoter utility
Trait-subtrait Area: Among other uses this promoter sequence could be useful to improve:

Source- C/N partitioning, transport of amino acids, source enhancement
Yield- Total yield
Quality- Amino acids, carbohydrates, Optimize C3-C4 transition

Utility: Sequence most useful to overexpress genes important in vascular maintenance and
transport in and out of the phloem and xylem.

| | |
|---|---|
| Construct: | G0013 |
| Promoter Candidate I.D.: | 1768610 (Old ID: 35139302) |
| cDNA ID: | 12679922 (Old IDs: 12328210, 4937586.) |
| T1 lines expressing (T2 seed): | SR00423-01, -02, -03, -04, -05 |

Promoter Expression Report # 4
Report Date: March 6, 2003
Promoter Tested In: Arabidopsis thaliana, WS ecotype
Spatial expression Summary:

| | |
|---|---|
| Flower | (H)sepal, (L)epidermis |
| Embryo | (H)suspensor, (H)preglobular, (H)globular, (M)heart, (M)torpedo, (L)late, (L)mature, |
| | (L)hypophysis |
| Ovule | Pre fertilization: (M)outer integument, (H)funiculus |
| | Post fertilization: (M)outer integument, (H)zygote |
| Embryo | (H)hypocotyl, (H)epidermis, (H)cortex, (H)stipules, (L)lateral root, (H)initials, |
| | (H)lateral root cap |
| Stem | (L)epidermis |

Observed expression patterns: T1 Mature: Strong expression was seen in 4-cell through
heart stage embryo with decreasing expression in the torpedo stage; preferential expression in the
root and shoot meristems of the mature embryo. Strong expression was seen in the outer
integument and funiculus of developing seed. T2 Seedling: Strong expression was seen in
epidermal and cortical cells at the base of the hypocotyl. Strong expression was seen in stipules
flanking rosette leaves. Low expression was seen in lateral root initials with increasing expression
in the emerging lateral root cap. T2 Mature-Same expression patterns were seen as T1 mature
plants with weaker outer integument expression in second event. Both lines show additional
epidermal expression at the inflorescence meristem, pedicels and tips of sepals in developing
flowers. T3 seedling expression - same expression
Expected expression pattern: Expression in ovules

| | |
|---|---|
| Selection Criteria: | Greater than 50x up in pi ovule microarray |
| Gene: | Lipid transfer protein-like |
| GenBank: | NM_125323 Arabidopsis thaliana lipid transfer protein 3 |
| | (LTP 3) (At5g59320) mRNA, complete cds, |
| | gi\|30697205\|ref\|NM_125323.2\|[30697205] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |

Bidirectionality: NO Exons: NO Repeats: None noted
Promoter utility
Trait-subtrait Area: Among other uses this promoter sequence could be useful to improve:

Water use efficiency- Moisture stress, water use efficiency, ovule/seed
abortion Seed- test weight, seed size
Yield- harvest index, total yield
Quality- amino acids, carbohydrate, protein total oil, total seed composition

| | |
|---|---|
| Construct: | YP0097 |
| Promoter Candidate I.D: | 11768657 (Old ID: 35139702) |
| cDNA_ID | 12692181 (Old IDs: 12334169, 1021642) |
| T1 lines expressing (T2 seed): | SR00706-01, -02 |

Promoter Expression Report # 5
Report Date: March 6, 2003
Promoter Tested In: Arabidopsis thaliana, WS ecotype
Spatial expression summary:

| | |
|---|---|
| Ovule | Pre-fertilization: (L)inner integument |
| | Post-fertilization: (H)inner integument, (M)endothelium |
| Primary Root | (H)endodermis |

Observed expression pattern: GFP is expressed in the endosperm of developing seeds and pericycle
cells of seedling roots. GFP level rapidly increases following fertilization, through mature endosperm
cellularization. GFP is also expressed in individual pericycle cells. T1 and T2 mature: Same expression
pattern was observed in T1 and T2 mature plants. Closer examination of the images reveals that GFP is
expressed in the endothelium of ovules which is derived from the inner most layer of the inner
integuments. Lower levels of expression can be seen in the maturing seeds which is consistent with
disintegration of the endothelium layer as the embryo enters maturity. T2 seedling: Expression appears
to be localized to the endodermis which is the third cell layer of seedling root not pericycle as previously
noted. T3 seedlings: Low germination. No expression was observed in the few surviving seedlings.
Expected expression pattern: Expression in ovules

| | |
|---|---|
| Selection Criteria: | Greater than 50x up in pi ovule microarray |
| Gene: | palmitoyl-protein thioesterase |
| GenBank: | NM_124106 Arabidopsis thaliana palmitoyl protein thioesterase |
| | precursor, putative (At5g47350) mRNA, complete |
| | cdsgi\|30695161\|ref\|NM_124106.2\|[30695161] |

Source Promoter Organism: Arabidopsis thaliana WS

| | |
|---|---|
| Vector: | pNewbin4-HAP1-GFP. |
| Marker Type: | (X) GFP-ER |
| Generation Screened: | (X) T1 Mature (X) T2 Seedling (X) T3 Mature (X) T3 Seedling |
| Marker Intensity: | (X) High □ Med □ Low |

Bidirectionality: NO Exons: NO Repeats: None Noted
Promoter utility
Trait - Sub-trait Area: Among other uses this promoter sequence could be useful to improve:

Seed - ovule/seed abortion, seed size, test weight, total seed
Composition - amino acids, carbohydrate, protein to oil composition

Utility: Promoter useful for increasing endosperm production or affecting compositional changes in the
developing seed. Should also have utility in helping to control seed size.

| | |
|---|---|
| Construct: | YP0111 |
| Promoter Candidate I.D: | 11768845 (Old ID: 4772159) |
| cDNA ID 13619323 (Old IDs: | 12396169, 4772159) |
| T1 lines expressing (T2 seed): | SR00690-01, -02 |

Promoter Expression Report # 6
Report Date: March 6, 2003
Promoter Tested In: Arabidopsis thaliana, WS ecotype
Spatial expression summary:

| | |
|---|---|
| Stem | (H)epidermis, (H)cortex |
| Hypocotyl | (H)epidermis, (H)cortex |
| Silique | (H)style, (H)carpel, (H)septum, (H)epidermis |
| Leaf | (M)mesophyll, (M)epidermis |

Observed expression patterns: Strong GFP expression exists throughout stem epidermal and cortical
cells in T1 mature plants. GFP expression exhibits polarity in T2 seedling epidermal cells. First, it appears
in the upper part of the hypocotyl near cotyledonary petioles, increasing toward the root, and in the abaxial
epidermal cells of the petiole. An optical section of the seedling reveals GFP expression in the cortical
cells of the hypocotyl. T2 mature: Same expression pattern was seen as in T1 mature with extension of
cortex and epidermal expression through to siliques. No expression was seen in placental tissues and
ovules. Additional expression was observed in epidermis and mesophyll of cauline leaves. T3 seedling:
Same as T2.
Expected expression pattern: Expression in ovules

| | |
|---|---|
| Selection Criteria: | Greater than 50x up in pi ovule microarray |
| Gene: | cytochrome P450 homolog |
| GenBank: | NM_104570 Arabidopsis thaliana cytochrome P450, putative |
| | (At1g57750) mRNA, complete cds, |
| | gi\|30696174\|ref\|NM_104570.2\|[30696174] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |

Bidirectionality: NO Exons: NO Repeats: None Noted
Promoter utility
Trait - Sub-trait Area: Among other uses this promoter sequence could be useful to improve:

Water use efficiency - moisture stress, water use efficiency, ovule/seed abortion
Seed - test weight, seed size
Yield - harvest index, total yield
Composition - amino acids, carbohydrate, protein total oil, total seed

Utility: Useful when expression is predominantly desired in stems, in particular, the epidermis.

| | |
|---|---|
| Construct: | YP0104 |
| Promoter Candidate ID: | 11768842 |
| cDNA ID: 13612879 (Old IDs: | 12371683, 1393104) |
| T1 lines expressing (T2 seed): | SR00644-01, -02, -03 |

Promoter Expression Report # 7
Report Date: March 6, 2003
Promoter Tested In: Arabidopsis thaliana, WS ecotype
Spatial expression summary:

| | |
|---|---|
| Flower | (L)sepal, (L)petal, (L)silique, (L)vascular, (H)stomata, (L)pedicel |
| Silique | (L)vascular, (L)epidermis |
| Cotyledon | (H)stomata, (L)root hair |

Observed expression patterns: GFP expressed in the vasculature and guard cells of sepals
and pedicels in mature plants. GFP expressed in the guard cells of seedling cotyledons.
T2 mature: Stronger expression extended into epidermal tissue of siliques in proximal-distal
fashion. T3 seedling: Weak root hair expression was observed which was not observed in T2
seedlings; no guard cell expression observed. All epidermal tissue type expression was seen
with the exception of weak vasculature in siliques.
Expected expression pattern: Drought induced
Selection Criteria: Expression data (cDNAChip), >10 fold induction under drought condition.
Screened under non-induced condition.

| | |
|---|---|
| Gene: | Unknown protein; At5g43750 |
| GenBank: | NM_123742 Arabidopsis thaliana expressed protein |
| | (At5g43750) mRNA, complete cds, |
| | gi\|30694366\|ref\|NM_123742.2\|[30694366] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |

Bidirectionality: NO Exons: NO Repeats: None noted
Promoter utility
Trait - Subtrait Area: Among other uses this promoter sequence could be useful to improve:

Water use efficiency - Heat

| | |
|---|---|
| Construct: | YP0075 |
| Promoter Candidate I.D: | 11768626 (Old ID: 35139358) |
| cDNA ID: 13612919 (Old IDs: | 12694633, 5672796) |
| T1 lines expressing (T2 seed): | SR00554-01, -02 |

Promoter Expression Report # 8
Report Date: March 6, 2003
Promoter Tested In: Arabidopsis thaliana, WS ecotype
Spatial expression summary:

| | |
|---|---|
| Flower | (L)receptacle, (L)vascular |
| Leaf | (H)vascular, (H)epidermis |
| Root | (M)phloem |
| Cotyledon | (M)vascular, (M)hydathode |
| Primary Root | (L)epidermis, (M)vascular |

Observed expression patterns: Expression was seen at the receptacle and vasculature of
immature flower and leaf, and phloem of seedling root. T2 mature: Similar to T1 expression.
Strong expression was seen in vascular tissues on mature leaves. Vascular expression in flowers
was not observed as in T1. T3 seedling: Similar to T2 seedling expression.
Expected expression pattern: Vascular tissues; The SUC2 promoter directed
expression of GUS activity with high specificity to the phloem of all green tissues of
Arabidopsis such as rosette leaves, stems, and sepals.

| | |
|---|---|
| Selection Criteria: | Arabidopsis public; Planta 1995; 196: 564-70 |
| Gene: | “Sugar Transport” SUC2 |
| GenBank: | NM_102118 Arabidopsis thaliana sucrose transporter |
| | SUC2 (sucrose-proton transporter) (At1g22710) mRNA, complete |
| | cds, gi\|30688004\|ref\|NM_102118.2\|[30688004] |

Source Promoter Organism: Arabidopsis thaliana WS

| | |
|---|---|
| Vector: | Newbin4-HAP1-GFP |
| Marker Type: | X GFP-ER |
| Generation Screened: | X T1 Mature X T2 Seedling X T3 Mature X T3 Seedling |
| Bidirectionality: NO | Exons: NO Repeats: None Noted |
| Promoter utility | |

| | |
|---|---|
| Trait - Sub-trait Area: | Among other uses this promoter sequence could be useful to improve: |
| | Source - Source enhancement, C/N partitioning |

Utility: Useful for loading and unloading phloem.

| | |
|---|---|
| Construct: | YP0016 |
| Promoter Candidate I.D: | 11768612 (Old ID: 35139304) |
| cDNA ID | 13491988 (Old IDs: 6434453, 12340314) |
| T1 lines expressing (T2 seed): | SR00416-01, -02, -03, -04, -05 |

Promoter Expression Report # 9
Report Date: March 6, 2003
Promoter Tested In: Arabidopsis thaliana, WS ecotype
Spatial expression summary:

| | |
|---|---|
| Flower | (L)inflorescence, (H)pedicel, (H)vascular |
| Stem | (L)phloem |
| Leaf | (L)vascular |
| Ovule | Pre fertilization: (H)chalaza end of embryo sac |
| Hypocotyl | (M)vascular, (M)phloem |
| Cotyledon | (M)vascular, (M)phloem |
| Root | (H)vascular, (H)pericycle, (H)phloem |

Observed expression patterns: GFP expressed in the stem, pedicels and leaf vasculature of
mature plants and in seedling hypocotyl, cotyledon, petiole, primary leaf and root.
Expected expression pattern: Phloem of the stem, xylem-to-phloem transfer tissues, veins of
supplying seeds, vascular strands of siliques and in funiculi. Also expressed in the vascular
system of the cotyledons in developing seedlings. T2 mature: Same as T1 mature. T3 seedling:
Same as T2 seedling.

| | |
|---|---|
| Selection Criteria: | Arabidopsis public PNAS 92, 12036-12040 (1995) |
| Gene: | AAP2 (X95623) |
| GenBank: | NM_120958 Arabidopsis thaliana amino acid permease 2 (AAP2) |
| | (At5g09220) mRNA, complete cds, |
| | gi\|30682579\|ref\|NM_120958.2\|[30682579] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |
| Bidirectionality: FAILS | Exons: FAILS Repeats: None Noted |

Promoter Utility

| | |
|---|---|
| Trait - Sub-trati Area: | Among other uses this promoter sequence could be useful to improve: |
| Trait Area: | Seed - Seed enhancement |
| | Source - transport amino acids |
| | Yield - harvest index, test weight, seed size, |
| | Quality - amino acids, carbohydrate, protein, total seed composition |
| Utility: | |

| | |
|---|---|
| Construct: | YP0094 |
| Promoter Candidate I.D: | 11768636 (Old ID: 35139638) |
| cDNA ID: | 13609817 (Old IDs: 7076261, 12680497) |
| T1 lines expressing (T2 seed): | SR00641-01, -02 |

Promoter Expression Report # 10
Report Date: March 6, 2003
Promoter Tested In: Arabidopsis thaliana, WS ecotype
Spatial expression summary:

| | |
|---|---|
| Flower | (L)sepal, (L)pedicel, (L)vascular |
| Silique | (H)stomata |
| Hypocotyl | (M)epidermis |
| Primary Leaf | (H)stomata |
| Root | (H)epidermis, (H)root hairs |

Observed expression pattern: T1 mature: GFP expression was seen in the guard cells of
pedicles and mature siliques. Weak expression was seen in floral vasculature. T2 seedling: Strong
expression observed in epidermis and root hairs of seedling roots (not in lateral roots) and guard
cells of primary leaves. T2 mature: Similar to T1 plants. T3 seedling: Similar to T2 seedling.
Screened under non-induced conditions.
Expected expression pattern: As described by literature. Expressed preferentially in the root,
not in mature stems or leaves of adult plants (much like AGL 17); induced by KNO3 at 0.5 hr
with max at 3.5 hr

| | |
|---|---|
| Selection Criteria: | Arabidopsis Public; Science 279, 407-409 (1998) |
| Gene: | ANR1, putative nitrate inducible MADS-box protein; |
| GenBank: | NM_126990 Arabidopsis thaliana MADS-box protein ANR1 |
| | (At2g14210) mRNA, complete cds |
| | gi\|22325672\|ref\|NM_126990.2\|[22325672] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |
| Bidirectionality: NO | Exons: NO Repeats: None Noted |
| Promoter Utility | |

| | |
|---|---|
| Trait - Sub-trait Area: | Among other uses this promoter sequence could be useful to improve: |
| | Yield - Heterosis, general combining ability, specific combining ability |

| | |
|---|---|
| Construct: | YP0033 |
| Promoter Candidate I.D: | 13148205 (Old ID: 35139684) |
| cDNA ID: | 12370148 (Old IDs: 7088230, 12729537) |
| T1 lines expressing (T2 seed): | SRXXXXX-01, |

Promoter Expression Report # 11
Report Date: March 6, 2003
Promoter Tested In: Arabidopsis thaliana, WS ecotype
Spatial expression summary:

| | |
|---|---|
| Flower | (H)epidermis, (H)sepal, (H)petal, (H)vascular |
| Stem | (L)vascular |
| Hypocotyl | (L)epidermis, (H)phloem |
| Cotyledon | (L)epidermis, (M)stomata, (L)vascular |
| Root | (H)phloem |

Observed expression pattern: Strong GFP expression was seen in the epidermal layer
and vasculature of the sepals and petals of developing flowers in mature plants and
seedlings. T2 mature: Expression was similar to T1 mature plants. Vascular expression
in the stem was not observed in T1 mature. T3 Seedling: Same expression seen as T2
seedling expression
Expected expression pattern: Predominantly expressed in the phloem.

| | |
|---|---|
| Selection Criteria: | Arabidopsis public: Deeken, R. The Plant J.(2000) 23(2), 285-290 |
| | Geiger, D. Plant Cell (2002) 14, 1859-1868 |

| | |
|---|---|
| Gene: | potassium channel protein AKT3 |

GenBank: NM_118342 Arabidopsis thaliana potassium channel (K+ transporter2)(AKT2)
(At4g22200) mRNA, complete cds,
gi|30685723|ref|NM_118342.2|[30685723]
Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |
| Bidirectionality: NO | Exons: NO Repeats: None Noted |

| | |
|---|---|
| Trait - Sub-trait Area: | Among other uses this promoter sequence could be useful to improve: |
| | Nutrient - Low nitrogen tolerance; Nitrogen use efficiency; Nitrogen |
| | utilization |
| Utility: | |

| | |
|---|---|
| Construct: | YP0049 |
| Promoter Candidate I.D: | 11768643 (Old ID: 6452796) |
| cDNA ID | 12660077 (Old IDs: 7095446, 6452796) |
| T1 lines expressing (T2 seed): | SR00548-01, -02, -03 |

Promoter Expression Report # 12
Report Date: March 6, 2003
Promoter Tested In: Arabidopsis thaliana, WS ecotype
Spatial expression summary:

| | |
|---|---|
| Flower | (L)pedicel, (L)sepal, (L)vascular |
| Leaf | (M)petiole, (M)vascular |
| Cotyledon | (H)stomata, (M)petiole, (H)vascular |
| Primary Leaf | (L)vascular, (L)petiole |
| Root | (H)root hair |

Observed expression pattern: GFP expression was detected in the vasculature of sepals,
pedicel, and leaf petiole of immature flowers. Also weak guard cell expression existed in
sepals. Strong GFP expression was seen in guard cells and phloem of cotyledons, and
upper root hairs at hypocotyl root transition zone. T2 mature: Same as T1 mature. T3
seedling: Same as T2seedling.
Expected expression pattern: Shoot apical meristems

| | |
|---|---|
| Selection Criteria: | Greater than 5x down in stm microarray |
| Gene: | AP2 domain transcription factor |
| GenBank: | NM_129594 Arabidopsis thaliana AP2 domain transcription |
| | factor, putative(DRE2B) (At2g40340) mRNA, complete cds, |
| | gi\|30688235\|ref\|NM_129594.2\|[30688235] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |
| Bidirectionality: NO | Exons: FAILS Repeats: None Noted |
| Promoter Utility | |

| | |
|---|---|
| Trait Area: | Among other uses this promoter sequence could be useful to improve: |
| | Cold, PG&D, |
| Sub-trait Area: | Cold germination & vigor, plant size, growth rate, plant development |
| Utility: | |

| | |
|---|---|
| Construct: | YP0060 |
| Promoter Candidate I.D: | 11768797 (Old ID: 35139885) |
| cDNA ID: | 13613553 (Old IDs: 4282588, 12421894) |
| T1 lines expressing (T2 seed): | SR00552-02, -03 |

Promoter Expression Report # 13
Report Date: March 6, 2003
Promoter Tested In: Arabidopsis thaliana, WS ecotype
Spatial expression summary:
Ovule Post-fertilization: (H)endothelium, (H)micropyle, (H)chalaza
Observed expression pattern: T1 and T2 mature: Strong expression was seen in the mature
inner integument cell layer, endothelium, micropyle and chalaza ends of maturing ovules.
Expression was not detected in earlier stage ovules. T2 and T3 seedling expression: None
Expected expression pattern: Primarily in developing seeds

| | |
|---|---|
| Selection Criteria: | Arabidopsis public; Mol. Gen. Genet. 244, 572-587 (1994) |
| Gene: | plasma membrane H(+)-ATPase isoform AHA10; |
| GenBank: | NM_101587 Arabidopsis thaliana ATPase 10, plasma membrane- |
| | type (proton pump 10) (proton-exporting ATPase), putative |
| | (At1g17260) mRNA, complete cds, gi\|18394459\| |

Source Promoter Organism: Arabidopsis thaliana WS

| | |
|---|---|
| Vector: | pNewbin4-HAP1-GFP. |
| Marker Type: | X GFP-ER |
| Generation Screened: | X T1 Mature X T2 Seedling X T3 Mature X T3 Seedling |

Bidirectionality: FAILS Exons: FAILS Repeats: None Note

| | |
|---|---|
| Trait Area: | Among other uses this promoter sequence could be useful to improve: |
| | Seed - Endosperm cell number and size, endosperm granule number/size, seed |
| | enhancement |
| | Yield - harvest index, test weight, seed size |
| | Quality - protein, total oil, total seed composition, composition |
| Utility: | |

| | |
|---|---|
| Construct: | YP0092 |
| Promoter Candidate I.D: | 13148193 (Old ID: 35139598) |
| cDNA ID | 12661844 (Old ID: 4993117) |
| T1 lines expressing (T2 seed): | SR00639-01, -02, -03 |

Promoter Expression Report # 14
Report Date: March 6, 2003
Promoter Tested In: Arabidopsis thaliana, WS ecotype
Spatial expression summary:

| | |
|---|---|
| Flower | (L)silique |
| Silique | (L)medial vasculature, (L)lateral vasculature |

Observed expression pattern: GFP expressed in the medial and lateral vasculature of
pre-fertilized siliques. Expression was not detected in the older siliques or in T2
seedlings. T2 mature: Weak silique vasculature expression was seen in one of two
events. T3 seedling: Same as T2 seedling, no expression was seen.
Expected expression pattern: Expression in ovules

| | |
|---|---|
| Selection Criteria: | Greater than 50x up in pi ovule microarray |
| Gene: | expressed protein; protein id: At4g15750.1, hypothetical |
| | protein |
| GenBank: | NM_117666 Arabidopsis thaliana expressed protein |
| | (At4g15750) mRNA, complete cds gi\|18414516\|ref\|NM_117666.1\|[18414516] |

Source Promoter Organism: Arabidopsis thaliana WS

| | |
|---|---|
| Vector: | pNewbin4-HAP1-GFP |
| Marker Type: | X GFP-ER |
| Lines Screened: | n = 3 |
| Lines Expressing: | n = 3 |
| Generation Screened: | X T1 Mature X T2 Seedling X T3 Mature X T3 Seedling |
| Bidirectionality: NO | Exons: NO Repeats: None Noted |
| Promoter utility | |
| Trait - Sub-trait Area: | Among other uses this promoter sequence could be useful to |
| | improve: |
| | Water use efficiency - Moisture stress at seed set, Moisture stress at seed fill, |
| | water use efficiency, Ovule/seed abortion |
| | Seed - test weight, seed size |
| | Yield - harvest index, , total yield |
| | Quality - amino acids, carbohydrate, protein, total oil, total seed composition |

| | |
|---|---|
| Construct: | YP0113 |
| Promoter Candidate I.D: | 13148162 (Old ID: 35139698) |
| cDNA ID: | 12332135 (Old ID: 5663809) |
| T1 lines expressing (T2 seed): | SR00691-01, -03 |

Promoter Expression Report # 15
Report Date: March 6, 2003
Promoter Tested In: Arabidopsis thaliana, WS ecotype
Spatial expression summary:

| | |
|---|---|
| Flower | (L)silique |
| Silique | (L)medial vasculature, (L)lateral vasculature, (H)guard cells |
| Rosette leaf | (H)guard cell |

Observed expression pattern: GFP expressed in the medial and lateral vasculature of
pre-fertilized siliques. Expression was not detected in older siliques. Guard cell
expression was seen throughout pre-fertilized and fertilized siliques. T2 seedling: No
expression was seen. T2 mature expression: Similar to T1 mature expression. T3 seedling:
Guard cell expression not seen in T2 seedlings, however it is in the same tissue type observed in
mature plants of previous generation.
Expected expression pattern: Strong activity in the inner endosperm tissue of
developing seeds and weak activity in root tips.

| | |
|---|---|
| Selection Criteria: | Arabidopsis public; Plant Mol. Biol. 39, 149-159 (1999) |
| Gene: | Alanine aminotransferase, AlaAT |

GenBank: NM_103859 Arabidopsis thaliana abscisic acid responsive elements-
binding factor (At1g49720) mRNA, complete
cdsgi|30694628|ref|NM_103859.2|[30694628]-
INCORRECT (L.M. 10/14/03)
AAK92629 - CORRECT (LM 10/14/03)
Putative alanine aminotransferase [Oryza sativa]
gi|15217285|gb|AAK92629.1|AC079633_9[15217285]
Source Promoter Organism: Rice

| Vector: | pNewbin4-HAP1-GFP. | |
|---|---|---|
| Marker Type: | X GFP-ER | |
| Generation Screened: | X T1 Mature | X T2 Seedling X T3 Mature X T3 Seedling |
| Bidirectionality: NO | Exons: NO | Repeats: None Noted |

Promoter utility
Trait Area: Among other uses this promoter sequence could be useful to improve:
Seed, source, yield, quality
Sub-trait Area: Seed enhancement, transport amino acids, harvest index, test
weight, seed size, amino acids, carbohydrate, protein, total seed composition

| | |
|---|---|
| Construct: | YP0095 |
| Promoter Candidate ID: | 13148198 (Old ID: 35139658) |
| cDNA ID: | 6795099 in rice |
| T1 lines expressing (T2 seed): | SR00642-02, -03 |

Promoter Expression Report # 16
Report Date: March 6, 2003
Promoter Tested In: Arabidopsis thaliana, WS ecotype
Spatial expression summary:

| | |
|---|---|
| Ovule | Pre-fertilization: (M)gametophyte, (M)embryo sac |
| Root | (H)epidermis, (M)pericycle, (H)root hairs |
| Lateral root | (H)flanking cells |

Observed expression patterns: GFP expressed in the egg cell and synergid cell of
female gametophyte in early ovule development. It expressed in polarizing embryo sac in
later stages of pre-fertilized ovule development. No expression was seen in fertilized
ovules. GFP expressed throughout the epidermal cells of seedling roots. It also expressed
in flanking cells of lateral root primordia.
T2 mature: Same as T1 mature. T3 seedling: Same as T2 seedling
Expected expression pattern: Expression in ovules

| | |
|---|---|
| Selection Criteria: | Greater than 50x up in pi ovule microarray |
| Gene: | Senescence-associated protein homolog |

GenBank: NM_119189 Arabidopsis thaliana senescence-associated protein family
(At4g30430) mRNA, complete cds, gi|18417592|ref|NM_119189.1|[18417592]
Source Promoter Organism: Arabidopsis thaliana WS

| | |
|---|---|
| Vector: | pNewbin4-HAP1-GFP |
| Marker Type: | X GFP-ER |
| Generation Screened: | XT1 Mature X T2 Seedling X T3 Mature X T3 Seedling |

Bidirectionality: NO Exons: NO Repeats: None Noted
Promoter utility
Trait Area: Among other uses this promoter sequence could be useful to improve:
Water use efficiency, seed, yield
Sub-trait Area: Moisture stress, water use efficiency, ovule/seed abortion, harvest index,
test weight, seed size, total yield, amino acids, carbohydrate, proteintotail oil, total seed
composition

| | |
|---|---|
| Construct: | YP0102 |
| Promoter Candidate I.D: | 11768651 (Old ID: 35139696) |
| cDNA ID: | 13613954 (Old IDs: 12329268, 1382001) |
| T1 lines expressing (T2 seed): | SR00643-01, -02 |

Promoter Expression Report # 17
Report Date: March 6, 2003
Promoter Tested In: Arabidopsis thaliana, WS ecotype
Spatial expression summary:

| | |
|---|---|
| Ovule | Pre-fertilization: (H)inner integument |
| | Post-fertilization: (H)inner integument, (M)outer integument, |
| | (M)seed coat |
| Primary Root | (L)root hair |

Observed expression pattern: GFP expressed in the inner integuments of pre-fertilized
and fertilized ovules. Female gametophyte vacuole seen as dark oval. T2 mature: Same
expression was seen as T1 with additional expression observed in similar tissue. GFP
expressed in the outer integument and seed coat of developing ovules and seed. T3
seedling expression: GFP expression was seen in a few root hairs.
Expected expression pattern: Expression in ovules

| | |
|---|---|
| Selection Criteria: | Greater than 50x up in pi ovule microarray |
| Gene: | putative protease inhibitor |

GenBank: NM_129447 Arabidopsis thaliana protease inhibitor - related (At2g38900)
mRNA, complete cds, gi|30687699|ref|NM_129447.2|[30687699]
Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |
| Bidirectionality: NO | Exons: FAILS Repeats: None Noted |

Promoter utility
Trait Area: Among other uses this promoter sequence could be useful to improve:
Water use efficency, seed, yield
Sub-trait Area: Moisture stress, water use efficiency, ovule/seed abortion, harvest index,
test weight, seed size, total yield, amino acids, carbohydrate, proteintotail oil, total seed
composition.

| | |
|---|---|
| Construct: | YP0103 |
| Promoter Candidate I.D: | 13148199(Old ID: 35139718) |
| cDNA ID: 4905097 (Old ID: | 12322121, 1387372) |
| T1 lines expressing (T2 seed): | SR00709-01, -02, -03 |

Promoter Expression Report # 18
Report Date: March 6, 2003
Promoter Tested In: Arabidopsis thaliana, WS ecotype
Spatial expression summary:

| | |
|---|---|
| Embryo | (H)mature, (H)late |
| Ovule | (H)endothelium |
| Primary root | (L)root hair |

Observed expression pattern: Low levels of GFP expression were detected in late torpedo
stage with highest levels in the mature and late embryo. High GFP expression was detected in
late endosperm stage in endothelium layer of developing seed. T2 mature: Same as T1 mature.
T3 seedling: GFP was detected in a few root hairs not observed in T2 seedlings.
Expected expression pattern: Embryo and seed

| | |
|---|---|
| Selection Criteria: | Arabidopsis public; Rossak, M. Plant Mol. Bio. 2001.46: 717 |
| Gene: | fatty acid elongase 1; FAE1 |
| GenBank: | NM_119617 Arabidopsis thaliana fatty acid elongase 1 (FAE1) |
| | (At4g34520) mRNA, complete cds, |
| | gi\|30690063\|ref\|NM_119617.2\|[30690063] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |

Bidirectionality: NO Exons: NO Repeats: Not Done
Promoter utility

| | |
|---|---|
| Trait - Sub-trait Area: | Among other uses this promoter sequence could be useful to improve: |
| | Seed - Ovule/seed abortion, seed enhancement, seed size |
| | Yield |

| | |
|---|---|
| Construct: | YP0107 |
| Promoter Candidate I.D: | 13148252 (Old ID: 35139824) |
| cDNA ID: | 12656458 (Old ID: 1815714) |
| T1 lines expressing (T2 seed): | SR00646-01, -02 |

Promoter Expression Report # 19
Report Date: March 6, 2003
Promoter Tested In: Arabidopsis thaliana, WS ecotype
Spatial expression summary:

| | |
|---|---|
| Ovule | Pre-fertilization: (M)gametophyte, (M)embryo sac |
| | Post-fertilization: (H)zygote |

Observed expression pattern: GFP expressed in the developing female gametophyte of
unfertilized ovules and the degenerated synergid cell of the fertilized ovule hours after
fertilization. No expression was observed in T2 seedlings. T2 mature: Similar expression as T1
mature. T3 seedling: Root expression in one of two events was not observed in T2 seedlings.
No expression was observed in the second line which is consistent with T2 seedling expression.
Expected expression pattern: Expression in ovules

| | |
|---|---|
| Selection Criteria: | Greater than 50x up in pi ovule microarray |
| Gene: | Hypothetical protein |
| GenBank: | NM_112033 Arabidopsis thaliana expressed protein (At3g11990) |
| | mRNA, complete cds |

gi|18399438|ref|NM_112033.1|[18399438]
Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |

Bidirectionality: NO Exons: FAILS Repeats: None Noted
Promoter utility
Trait Area: Among other uses this promoter sequence could be useful to improve:
Water use efficency, seed, yield

| | |
|---|---|
| Sub-trait Area: | Moisture stress, water use efficiency, ovule/seed abortion, harvest index, |
| | test weight, seed size, total yield, amino acids, carbohydrate, proteintotail |
| | oil, total seed composition. |

| | |
|---|---|
| Construct: | YP0110 |
| Promoter Candidate I.D: | 13148212 (Old ID: 35139697) |
| cDNA ID: | 13604221 (Old IDs: 12395818, 4772042) |
| T1 lines expressing (T2 seed): | SR00689-02, -03 |

Promoter Expression Report # 20
Report Date: March 6, 2003
Promoter Tested In: Arabidopsis thaliana, WS ecotype
Spatial expression summary:

| | |
|---|---|
| Flower | (L)silique |
| Silique | (M)medial vasculature, (M)lateral vasculature, (M)guard cells |

Observed expression pattern: GFP expressed in the medial and lateral vasculature of pre-
fertilized siliques. Expression was not detected in older siliques. Guard cell expression was seen
throughout pre-fertilized and fertilized siliques. T2 Mature: Same as T1 Mature. T2 seedling:
Same as T2 seedling.
Expected expression pattern: Expression in ovules

| | |
|---|---|
| Selection Criteria: | Greater than 50x up in pi ovule microarray |
| Gene: | hypothetical protein |
| GenBank: | NM_104488 Arabidopsis thaliana hypothetical protein |
| | (At1g56100) mRNA, complete cds |
| | gi\|18405686\|ref\|NM_104488.1\|[18405686] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |

Bidirectionality: NO Exons: FAILS Repeats: None Noted
Promoter Utility

| | |
|---|---|
| Trait Area: | Among other uses this promoter sequence could be useful to improve: |
| | Water use efficiency, seed, yield |

| | |
|---|---|
| Sub-trait Area: | Moisture stress at seed set, moisture stress at seed fill, water use efficiency, |
| | ovule/seed abortion, harvest index, test weight, seed size, total yield, amino |
| | acids, carbohydrate, protein, total oil, total seed composition, composition |
| Utility: | |

| | |
|---|---|
| Construct: | YP0112 |
| Promoter Candidate I.D: | 13148226 (Old ID: 35139719) |
| cDNA ID: | 12321680 (Old ID: 5662775) |
| T1 lines expressing (T2 seed): | SR00710-01, -02, -03 |

Promoter Expression Report # 21
Report Date: March 6, 2003
Promoter Tested In: Arabidopsis thaliana, WS ecotype
Spatial expression summary:
Silique (H)stigma, (H)transmitting tissue
Observed expression pattern: GFP expression was seen in the stigma and pollen transmitting
tract spanning the entire silique. No expression was detected in the T2 seedlings.
T2 Mature: Same as T1. T3 seedlings: No data
Expected expression pattern: Expression in ovules

| | |
|---|---|
| Selection Criteria: | Greater than 50x up in pi ovule microarray |
| Gene: | putative drought induced protein |
| GenBank: | NM_105888 Arabidopsis thaliana drought induced protein — |
| | related (At1g72290) mRNA, complete cds |
| | gi\|18410044\|ref\|NM_105888.1\|[18410044] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T3 Mature X T3 Seedling |

Bidirectionality: NO Exons: NO Repeats: None Noted
Promoter utility
Trait - Sub-trait Area: Among other uses, this promoter sequence could be useful to improve:
Water use efficiency - Moisture stress at seed set, Moisture stress at seed fill, water use
efficiency, Ovule/seed abortion
Utility: Interesting to think about using this promoter to drive a gene that would select against a specific
pollen type in a hybrid situation.

| | |
|---|---|
| Construct: | YP0116 |
| Promoter Candidate I.D: | 13148262 (Old ID: 35139699) |
| cDNA ID: | 12325134 (Old ID: 6403538) |
| T1 lines expressing (T2 seed): | SR00693-02, -03 |

Promoter Expression Report # 22
Report Date: March 8, 2003
Promoter Tested In: Arabidopsis thaliana, WS ecotype
Spatial expression summary:

| | |
|---|---|
| Flower | (H)pedicle |
| Silique | (M)vascular |
| Stem | (H)cortex |
| Ovule | Pre-fertilization: (H)outer integument, (M)chalaza |
| Hypocotyl | (H)cortex |
| Root | (H)epidermis, (H)atrichoblast, (H)cortex |

Observed expression pattern:
Strong GFP expression was seen in the adaxial surface of the pedicel and secondary
inflorescence meristem internodes. High magnification reveals expression in 2-3 cell
layers of the cortex. GFP expressed in the vasculature of silique, inner integuments, and
chalazal region of ovule. Expression was highest in the outer integuments of pre-
fertilized ovules decreasing to a few cells at the micropylar pole at maturity. Specific
expression was in the chalazal bulb region where mineral deposits are thought to be
accumulated for seed storage. GFP expressed in 2 cortical cell layers of the hypocotyl
from root transition zone to apex. At the apex, GFP is expressed at the base of the leaf
primordial and cotyledon. Root expression is specific to the epidermis and cortex. T2
Mature: Same as T1 mature. T3 seedling: Same expression as in T2 seedlings.
Expression is different in one seedling which has with weak root epidermal, weak
hypocotyl and stronger lateral root expression. This expression is variable within siblings
in this family.
Expected expression pattern: Expressed in ovules and different parts of seeds
Selection Criteria: Greater than 50x up in pi ovule microarray

| | |
|---|---|
| Gene: | hypothetical protein T20K18.24 |
| GenBank: | NM_117358 Arabidopsis thaliana expressed protein |
| | (At4g12890) |
| | mRNA, complete cds |
| | gi\|30682271\|ref\|NM_117358.2\|[30682271] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |
| Bidirectionality: NO | Exons: NO Repeats: NO |

Promoter utility
Trait-Sub-trait Area: Among other uses this promoter sequence could be useful to improve:

Water use efficiency - Moisture stress at seed set, Moisture stress at seed fill,
water use efficiency, ovule/seed abortion
Seed - harvest index, test weight, seed size
Yield - total yield
Quality - amino acids, carbohydrate, protein, total oil, total seed composition

| | |
|---|---|
| Construct: | YP0117 |
| Promoter Candidate I.D: | 11768655 (Old ID: 35139700) |
| cDNA I.D: | 13617054 (Old IDs: 12322571, 7074452) |
| T1 lines expressing (T2 seed): | SR00694-01, -02 |

Promoter Expression Report # 23
Report Date: March 8, 2003
Promoter Tested In: Arabidopsis thaliana, WS ecotype
Spatial expression summary:

| | |
|---|---|
| Flower | (L)silique |
| Silique | (L)carpel, (L)vascular |

Observed expression pattern: Low levels of GFP expressed in the medial and lateral
vasculature of developing pre-fertilized siliques.
T2 mature: No Expression. T3 seedling: No Expression.
Expected expression pattern: Expressed in ovules and different parts of seeds.

| | |
|---|---|
| Selection Criteria: | Greater than 50x up in pi ovule microarray |
| Gene: | Putative vacuolar processing enzyme |
| GenBank: | NM_112912 Arabidopsis thaliana vacuolar processing |
| | enzyme/asparaginyl endopeptidase —related |
| | (At3g20210) mRNA, complete cds |
| | gi\|30685671\|ref\|NM_112912.2\|[30685671] |

Source Promoter Organism: Arabidopsis thaliana WS

| | |
|---|---|
| Vector: | pNewbin4-HAP1-GFP |
| Marker Type: | X GFP-ER |
| Generation Screened: | X T1 Mature X T2 Seedling X T2 Mature X T3 Seedling |

Bidirectionality: NO Exons: NO Repeats: None Noted
Promoter utility

| Trait Area: | Among other uses this promoter sequence could be useful to improve: |
|---|---|
| | Water use efficiency - Moisture stress at seed set, Moisture stress at |
| | seed fill, water use efficiency, ovule/seed abortion |
| | Seed - harvest index, test weight, seed size |
| | Yield - total yield |
| | Quality - amino acids, carbohydrate, protein, total oil, total seed composition |

| | |
|---|---|
| Construct: | YP0118 |
| Promoter Candidate I.D: | 11768691 (Old ID: 35139754) |
| cDNA I.D: | 12329827 (Old ID: 4908806) |
| T1 lines expressing (T2 seed): | SR00711-01, -02, -03 |

Promoter Expression Report # 24
Report Date: March 9, 2003
Promoter Tested In: Arabidopsis thaliana, WS ecotype
Spatial expression summary:

| | |
|---|---|
| Flower | sepal, petal, silique |
| Silique | epidermis |
| Leaf | mesophyll, vascular, epidermis, margin |
| Hypocotyl | epidermis |
| Cotyledon | mesophyll, vascular epidermis |

Observed expression pattern: Screened under non-induced conditions. Strong GFP expression
was seen in epidermal and vasculature tissue of mature floral organs and leaves including
photosynthetic cells. GFP is expressed in two cell layers of the margin and throughout mesophyll
cells of mature leaf. GFP expressed in the epidermal cells of hypocotyl and cotyledons and
mesophyll cells. GFP expression in the leaf is non guard cell, epidermal specific.
Expected expression pattern: N induced, source tissue.

| | |
|---|---|
| Selection Criteria: | arabidopsis microarray-nitrogen |
| Gene: | hypothetical protein, auxin-induced protein-like |
| GenBank: | NM_120044 Arabidopsis thaliana auxin-induced (indole-3-acetic |
| | acid induced) protein, putative (At4g38840) mRNA, complete cds |
| | gi\|18420319\|ref\|NM_120044.1\|[18420319] |

Source Promoter Organism: Arabidopsis thaliana WS

| | |
|---|---|
| Vector: | pNewbin4-Hap1-GFP |
| Marker Type: | X GFP-ER |
| Generation Screened: | XT1 Mature X T2 Seedling X T3 Mature X T3 Seedling |

Bidirectionality: FAILS Exons: FAILS Repeats: None Noted
Promoter utility
Trait-Sub-trait Area: Among other uses this promoter sequence could be useful to improve:

Source - Photosynthetic efficiency
Yield - seed size

| | |
|---|---|
| Construct: | YP0126 |
| Promoter Candidate I.D: | 11768662 (Old ID: 35139721) |
| cDNA ID: | 12713856 (Old IDs: 12580379, 4767659) |
| T1 lines expressing (T2 seed): | SR00715-01, -02 |

Promoter Expression Report # 25
Report date: March 23, 2003
Promoter Tested In: Arabidopsis thaliana, WS ecotype
Spatial expression summary:

| | |
|---|---|
| Flower | (H)sepal, (H)anther |
| Silique | (M)vascular |
| Ovule | Post-fertilization: (M)inner integument, (M)chalaza, (M)micropyle |
| Stem | (H)Pith |
| Hypocotyl | (H)phloem |
| Cotyledon | (M)epidermis |
| Rosette Leaf | (H)hydathode |
| Primary Root | (H)phloem, (H)pericycle |
| Lateral root | (H)phloem |

Observed expression pattern: Expressed in the vasculature of sepal and connective tissue of
anthers in pre-fertilized flowers, inner integuments restricted to micropyle region, and chalazal
bulb of post-fertilized ovules. GFP expressed throughout the phloem of hypocotyl and root and
in pericycle cells in root differentiation zone. Screened under non-induced conditions.
T2 mature: Same expression as observed in T1 mature. In addition, silique vascular expression
was not observed in T1 mature. T3 seedling: Same expression as observed in T2 seedlings. In
addition, expression was observed in cotyledon epidermal and rosette leaf hydathode secretory
gland cells.
Expected expression pattern: nitrogen induced

| | |
|---|---|
| Selection Criteria: | Arabidopsis microarray |
| Gene: | probable auxin-induced protein |
| GenBank: | NM_119918 Arabidopsis thaliana lateral organ boundaries |
| | (LOB) domain family (At4g37540) mRNA, complete cds |
| | gi\|18420067\|ref\|NM_119918.1\|[18420067] |

| | |
|---|---|
| Source Promoter Organism: | Arabidopsis thaliana WS |
| Vector: | pNewBin4-HAP1-GFP |

| | |
|---|---|
| Marker Type: | X GFP-ER |
| Generation Screened: | X T1 Mature X T2 Seedling X T2 Mature X T3 Seedling |

Bidirectionality: NO Exons: NO Repeats: None Noted
Promoter Utility
Trait - Sub-trait Area: Among other uses this promoter sequence could be useful to improve:

Source - Photosynthetic efficiency
Yield - seed size

Utility:

| | |
|---|---|
| Construct: | YP0127 |
| Promoter Candidate I.D: | 13148197 (Old ID: 11768663) |
| cDNA I.D: | 13617784 (Old IDs: 12712729, 4771741) |
| T1 lines expressing (T2 seed): | SR00716-01, -02 |

Promoter Expression Report # 26
Report Date: March 17, 2003
Promoter Tested In: Arabidopsis thaliana, WS ecotype
Spatial expression summary:

| | |
|---|---|
| Silique | (L)vascular |
| Rosette Leaf | (H)stipule |
| Primary Root | (H)trichoblast, (H)atrichoblast |
| Cotyledon | (L)hydathode |

Observed expression pattern: Weak expression in vasculature of pre-fertilized siliques.
Expressed throughout epidermal cells of seedling root. T2 mature: Expression not
confirmed. T3 seedlings: Same expression as observed in T2 seedlings. In addition,
expression was observed in cotyledon epidermal and hydathode secretory gland cells.
Expected expression pattern: Inducible promoter - induced by different forms of stress
(e.g., drought, heat, cold).

| | |
|---|---|
| Selection Criteria: | Arabidopsis microarray-Nitrogen |
| Gene: | similar to SP\|P30986 reticuline oxidase precursor |
| | (Berberine-bridge-forming enzyme; Tetrahydroprotoberberine |
| | synthase) |
| | contains PF01565 FAD binding domain” |
| | product = “FAD-linked oxidoreductase family” |
| GenBank: | NM_102808 Arabidopsis thaliana FAD-linked |
| | oxidoreductase family (At1g30720) mRNA, complete cds |
| | gi\|30692034\|ref\|NM_102808.2\|[30692034] |

| | |
|---|---|
| Source Promoter Organism: | Arabidopsis thaliana WS |
| Vector: | pNewBin4-HAP1-GFP |

| | |
|---|---|
| Marker Type: | X GFP-ER |
| Generation Screened: | XT1 Mature X T2 Seedling X T2 Mature X T3 Seedling |
| Bidirectionality: NO | Exons: NO Repeats: NO |

Promoter utility
Trait - Sub-trait Area: Among other uses this promoter sequence could be useful to improve:

Water use efficiency - Heat

| | |
|---|---|
| Utility: | This promoter is useful for root nutrient uptake. |

| | |
|---|---|
| Construct: | YP0128 |
| Promoter Candidate I.D: | 13148257 (Old ID: 11769664) |
| cDNA I.D: | 13610584 (Old IDs: 12327909, 4807730) |
| T1 lines expressing (T2 seed): | SR00717-01, -02 |

Promoter Expression Report # 27
Report Date: March 23, 2003
Promoter Tested In: Arabidopsis thaliana, WS ecotype
Spatial expression summary:

| | |
|---|---|
| Flower | (L)stomata |
| Silique | (M)stomata |
| Stem | (L)stomata |
| Cotyledon | (L)mesophyll, (L)vascular, (M)hydathode |
| Rosette Leaf | (H)stomata, (H)hydathode |
| Primary Root | (L)root hairs |

Observed expression pattern: Expression specific to upper root hairs at hypocotyl root
transition zone and hydathode secretory cells of the distal cotyledon.
T1 mature: No T1 mature expression by old screening protocol
T2 mature: Guard cell and Hydathode expression same as T1 mature expression (new
protocol), T2 and T3 seedling expression.

| | |
|---|---|
| Expected expression pattern: | Shoot and root meristem |
| Selection Criteria: | Literature. Plant Cell 1998 10 231-243 |

| | |
|---|---|
| Gene: | CYP90B1, Arabidopsis steroid 22-alpha-hydroxylase |
| | (DWF4) |
| GenBank: | NM_113917 Arabidopsis thaliana cytochrome p450, |
| | putative (At3g30180) mRNA, complete cds |
| | gi\|30689806\|ref\|NM_113917.2\|[30689806] |

| | |
|---|---|
| Source Promoter Organism: | Arabidopsis thaliana WS |
| Vector: | pNewBin4-HAP1-GFP |

| | |
|---|---|
| Marker Type: | X GFP-ER |
| Generation Screened: | XT1 Mature XT2 Seedling X T2 Mature X T3 Seedling |

Bidirectionality: NO Exons: NO Repeats: None Noted
Promoter utility
Trait - Sub-trait Area: Among other uses, this promoter sequence could be useful to improve:

PG&D - Plant size, growth rate

| | |
|---|---|
| Utility: | Useful to increase biomass, root mass, growth rate, seed set |

| | |
|---|---|
| Construct: | YP0020 |
| Promoter Candidate I.D: | 11768639 (Old ID: 11768639) |
| cDNA I.D: | 12576899 (Old ID: 7104529) |
| T1 lines expressing (T2 seed): | SR00490-01, -02, -03, -04 |

Promoter Expression Report # 28
Report Date: March 23, 2003
Promoter Tested In: Arabidopsis thaliana, WS ecotype
Spatial expression summary:

| | |
|---|---|
| Flower | (L)pedicel, (M)vascular |
| Stem | (H)vascular, (H)pith |
| Silique | (H)septum, (H)vascular |
| Cotyledon | (H)vascular, (H)epidermis |
| Rosette Leaf | (H)vascular, (H)phloem |
| Primary Root | (H)vascular; (H)phloem |
| Lateral root | (H)vascular |

Observed expression pattern: T1 mature (old protocol-screened target tissue): No
expression observed. T2 seedling: Strong expression throughout phloem of hypocotyl,
cotyledons, primary rosette leaves and roots. Also found in epidermal cells of upper root
hairs at root transition zone. GFP expressed in a few epidermal cells of distal cotyledon.
T1 mature: (new protocol-screened all tissues): High expression found in silique
vasculature. T2 mature: Strong expression detected in inflorescence meristem and
silique medial vasculature. T3 seedling: Same expression as T2 seedlings, however no
cotyledon vascular expression was detected.

| | |
|---|---|
| Expected expression pattern: | Shoot and root meristem |
| Selection Criteria: | Plant Physiol. 2002 129: 1241-51 |

Gene: brassinosteroid-regulated protein (xyloglucan endotransglycosylase related
protein

| | |
|---|---|
| GenBank: | NM_117490 Arabidopsis thaliana xyloglucan |
| | endotransglycosylase (XTR7) (At4g14130) mRNA, |
| | complete cds gi\|30682721\|ref\|NM_117490.2\|[30682721] |

| | |
|---|---|
| Source Promoter Organism: | Arabidopsis thaliana WS |
| Vector: | pNewBin4-HAP1-GFP |

| | |
|---|---|
| Marker Type: | X GFP-ER |
| Generation Screened: | X T1 Mature XT2 Seedling X T2 Mature X T3 Seedling |

Bidirectionality: NO Exons: NO Repeats: None Noted
Promoter utility

| | |
|---|---|
| Trait Area: | Among other uses this promoter sequence could be useful to improve: |
| | PG&D - Plant size, growth rate |

Utility: Useful to increase biomass, root mass, growth rate

| | |
|---|---|
| Construct: | YP0022 |
| Promoter Candidate I.D: | 11768614 |
| cDNA I.D: | 12711515 (Old ID: 5674312) |
| T1 lines expressing (T2 seed): | SR00492-02, -03 |

Promoter Expression Report # 29
Report Date: March 23, 2003
Promoter Tested In: Arabidopsis thaliana, WS ecotype
Spatial expression summary:

| | |
|---|---|
| Flower | (M)sepal, (L)stomata |
| Silique | (M)stomata |
| Rosette Leaf | (H)stomata |
| Primary Root | (H)epidermis, (H)trichoblast, (H)root hair |

Observed expression pattern: Strong GFP expression in stomata of primary rosette
leaves and epidermal root hair trichoblast cells of seedlings. T1 mature: No expression
observed. T2 seedling: Same as T2 seedling expression. T2 mature: Guard cell and
weak vascular expression in flowers.
Expected expression pattern: embryo

| | |
|---|---|
| Selection Criteria: | Plant J 2000 21: 143-55 |
| Gene: | ABI3-interacting protein 2, AIP2 [Arabidopsis thaliana] |
| GenBank: | NM_122099 Arabidopsis thaliana zinc finger (C3HC4- |
| | type RING finger) protein family (At5g20910) mRNA, |
| | complete cds |
| | gi\|30688046\|ref\|NM_122099.2\|[30688046] |

| | |
|---|---|
| Source Promoter Organism: | Arabidopsis thaliana, WS |
| Vector: | pNewBin4-HAP1-GFP |
| Marker Type: | X GFP-ER |
| Generation Screened: | X T1 Mature XT2 Seedling X T2 Mature X T3 Seedling |

Bidirectionality: NO Exons: FAILS Repeats: None Noted
Promoter utility

| | |
|---|---|
| Trait - Sub-trait Area: | Among other uses this promoter sequence could be useful to improve: |
| | Water use efficiency - Drought, heat |

Utility: This promoter might be useful for enhancing recovery after growth under water
deprivation Also could be useful for nutrition uptake

| | |
|---|---|
| Construct: | YP0024 |
| Promoter Candidate I.D: | 11768616 |
| cDNA I.D: | 13614559 (Old IDs: 12324998, 5675795) |
| T1 lines expressing (T2 seed): | SR00494-01, -03 |

Promoter Expression Report # 30
Report Date: March 17, 2003
Promoter Tested In: Arabidopsis thaliana, WS ecotype
Spatial expression summary:

| | |
|---|---|
| Silique | (H)ovule |
| Ovule | Pre-fertilization: (H)outer integument, (H)funiculus |
| | Post-fertilization: (H)outer integument, (H)funiculus |
| Rosette Leaf | (H)vascular |
| Primary Root | (H)epidermis, (H)trichoblast, (H)root hair |
| Lateral root | (H)pericycle |

Observed expression pattern: Strong GFP expression in upper root hairs at root
transition zone and in distal vascular bundle of cotyledon. Low expression in pericycle
cells of seedling root. T1 mature: No expression observed. T3 seedling: Same as T2
seedling expression. T2 mature: GFP expression in funiculus of ovules as in connective
tissue between locules of anther.

| | |
|---|---|
| Expected expression pattern: | Root vasculature |
| Selection Criteria: | Helariutta, et al. 2000 Cell 101: 555-567 |
| Gene: | SHR (Short-root gene) |
| GenBank: | NM_119928 Arabidopsis thaliana short-root transcription |
| | factor (SHR) (At4g37650) mRNA, complete cds |
| | gi\|30691190\|ref\|NM_119928.2\|[30691190] |
| Source Promoter Organism: | Arabidopsis thaliana WS |
| Vector: | pNewBin4-HAP1-GFP |

| | |
|---|---|
| Marker Type: | X GFP-ER |
| Generation Screened: | X T1 Mature XT2 Seedling X T2 Mature X T3 Seedling |

Bidirectionality: NO Exons: NO Repeats: None Noted
Promoter utility

| | |
|---|---|
| Trait - Sub-trait Area: | Among other uses this promoter sequence could be useful to improve: |
| | Water use efficiency - Increase leaf water potential |
| | PG&D - increase root biomass, plant size |
| | Nutrient - nitrogen use efficiency, nitrogen utilization, low nitrogen |
| | tolerance |

Utility: This promoter might be a good promoter for root nutrition uptake, root biomass.

| | |
|---|---|
| Construct: | YP0028 |
| Promoter Candidate I.D: | 11768648 |
| cDNA I.D: | 12561142 (Old ID: 7093615) |
| T1 lines expressing (T2 seed): | SR00586-03, -04 |

Promoter Expression Report # 31
Report Date: March 23, 2003
Promoter Tested In: Arabidopsis thaliana, WS ecotype
Spatial expression summary:

| | |
|---|---|
| Flower | (L)stomata |
| Primary Root | (H)epidermis, (H)trichoblast, (H)atrichoblast, (H)root hairs |

Observed expression pattern: Strong GFP expression specific to epidermal root hair
trichoblast and atrichoblast cells throughout seedling root. Not expressed in lateral root.
T1 mature: No expression observed. T2 mature: Low guard cell expression in flower
not observed in T1 mature. T3 seedling expression: Same as T2 seedlings.
Expected expression pattern: localized to the lateral root cap, root hairs, epidermis and
cortex of roots.
Selection Criteria: Arabidopsis public; The roles of three functional sulfate transporters
involved in uptake and translocation of sulfate in Arabidopsis thaliana. Plant J. 2000
23: 171-82

| | |
|---|---|
| Gene: | Sulfate transporter |
| GenBank: | NM_116931 Arabidopsis thaliana sulfate transporter - |
| | related (At4g08620) mRNA, complete cds |
| | gi\|30680813\|ref\|NM_116931.2\|[30680813] |

| | |
|---|---|
| Source Promoter Organism: | Arabidopsis thaliana WS |
| Vector: | pNewBin4-HAP1-GFP |

| | |
|---|---|
| Marker Type: | X GFP-ER |
| Generation Screened: | XT1 Mature XT2 Seedling X T2 Mature X T3 Seedling |

Bidirectionality: NO Exons: NO Repeats: None Noted
Promoter utility

| | |
|---|---|
| Sub-trait Area: | Among other uses this promoter sequence could be useful to improve: |
| | Water use efficiency - Water potential, drought, moisture stress at seed set |
| | and seed fill, water use efficiency |
| | Nutrient - nitrogen use efficiency |

Utility: This is good promoter root nutrient uptake, increase root mass and water use efficiency

| | |
|---|---|
| Construct: | YP0030 |
| Promoter Candidate I.D: | 11768642 |
| cDNA I.D: | 12664333 (Old ID: 7079065) |
| T1 lines expressing (T2 seed): | SR00545-01, -02 |

Promoter Expression Report # 32
Report Date: March 24, 2003
Promoter Tested In: Arabidopsis thaliana, WS ecotype
Spatial expression summary:

| | |
|---|---|
| Cotyledon | (L)epidermis |
| Primary Root | (H)epidermis, (H)trichoblast, (H)atrichoblast |

Observed expression pattern: High GFP expression in epidermal cells of seedling root
from hypocotyl root transition to differentiation zone. Not observed in root tip. Low GFP
expression in epidermal cells of distal cotyledon.
T1 mature: No expression detected. T2 mature: Guard cell expression in stem, pedicles.
Low silique vascular expression. T3 seedling: Same as T2 seedlings.

| | |
|---|---|
| Expected expression pattern: | predominantly expressed in the phloem |
| Selection Criteria: | Ceres microarray data |
| Gene: | putative glucosyltransferase [Arabidopsis thaliana] |
| GenBank: | BT010327 Arabidopsis thaliana At2g43820 mRNA, |
| | complete cds gi\|33942050\|gb\|BT010327.1\|[33942050] |

| | |
|---|---|
| Source Promoter Organism: | Arabidopsis thaliana WS |
| Vector: | pNewBin4-HAP1-GFP |
| Marker Type: | X GFP-ER |
| Generation Screened: | X T1 Mature XT2 Seedling X T2 Mature X T3 Seedling |

Bidirectionality: NO Exons: NO Repeats: None Noted
Promoter utility

| | |
|---|---|
| Trait - Sub-trait Area: | Among other uses this promoter sequence could be useful to improve: |
| | Nutrient - nitrogen and phosphate uptake and |
| | transport |
| | Growth and Development - plant size, growth rate |

Utility: Promoter should be useful where expression in the root epidermis is important.
Expression appears to be in expanded or differentiated epidermal cells.

| | |
|---|---|
| Construct: | YP0054 |
| Promoter I.D: | 13148233 (Old ID: 11768644) |
| cDNA I.D: | 12348737 (Old ID: 1609253) |
| T1 lines expressing (T2 seed): | SR00549-01, -02 |

Promoter Expression Report # 34
Report Date: January 31, 2003
Promoter Tested In: Arabidopsis thaliana, WS ecotype
Spatial expression summary:

| | |
|---|---|
| Flower | (M)sepal, (M)style, (M)epidermis |
| Stem | (M)epidermis, (H)endodermis, (H)cortex |
| Leaf | (H)mesophyll, (H)epidermis |
| Hypocotyl | (H)epidermis, (H)vascular |
| Cotyledon | (H)epidermis, (H)mesophyll |
| Primary Root | (H)epidermis, (H)trichoblast, (H)atrichoblast, (H)vascular phloem, |
| | (H)Root cap, (H)root hairs |
| Lateral root | (H)vascular, (H)cap |

Observed expression pattern: GFP expressed in sepals, style of silique in immature flowers,
mesophyll, and epidermis of mature leaves. GFP expressed throughout epidermal layers of
seedling including root tissue. Also expressed in mesophyll and epidermal tissue in distal primary
leaf, and vasculature of root. Specific expression in meristematic zone of primary and lateral root.
T2 Mature: Same expression as
T1 mature: Additional images taken of stem expression.
T3 Seedling expression pattern: Same as T2 seedling expression.

| | |
|---|---|
| Expected expression pattern: | Shoot apical meristem |
| Selection Criteria: | Greater than 5x down in stm microarray |
| Gene: | Fructose-bisphosphate aldolase |
| GenBank: | NM_118786 Arabidopsis thaliana fructose-bisphosphate |
| | aldolase, putative (At4g26530) mRNA, complete |
| | cds gi\|30687252\|ref\|NM_118786.2\|[30687252] |

| | |
|---|---|
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature X T3 Seedling |

Bidirectionality: NO?? Exons: NO?? Repeats: None Noted
Promoter Utility

| | |
|---|---|
| Trait - Sub-trait Area: | Among other uses this promoter sequence could be useful to improve: |
| | PG&D - Plant size, growth rate, plant |
| | development |
| | Water use efficiency - |

Utility:

| | |
|---|---|
| Construct: | YP0050 |
| Promoter Candidate I.D: | 13148170 (Old ID: 11768794) |
| cDNA I.D: | 4909806 (Old IDs: 12340148, 1017738) |
| T1 lines expressing (T2 seed): | SR00543-01, -02 |

Promoter Expression Report # 35
Report Date: March 24, 2003
Promoter Tested In: Arabidopsis thaliana, WS ecotype
Spatial expression summary:

| | |
|---|---|
| Flower | (H)pedicel, (H)anther, (H)pollen, (H)vascular, (H)epidermis |
| Stem | (H)cortex, (L)vascular |
| Hypocotyl | (H)epidermis, (H)vascular, (H)phloem |
| Cotyledon | (H)vascular |
| Primary Root | (H)vascular, (H)phloem, (H)pericycle |

Observed expression pattern: High GFP expression throughout seedling vasculature
including root. Low Expression at the base of hypocotyls. Not detected in rosette leaves.
T1 mature: No expression observed. T3 seedling: Same as T2 seedling expression. T2
mature: Strong vascular and epidermal expression in floral pedicels and in developing
pollen sacs of anthers.
Expected expression pattern: xylem parenchyma cells of roots and leaves and in the
root pericycles and leaf phloem.
Selection Criteria: Arabidopsis public; The roles of three functional sulfate transporters
involved in uptake and translocation of sulfate in Arabidopsis thaliana. Plant J. 2000
23: 171-82

| | |
|---|---|
| Gene: | Sulfate transport |
| GenBank: | NM_121056 Arabidopsis thaliana sulfate transporter |
| | (At5g10180) mRNA, complete cds |
| | gi\|30683048\|ref\|NM_121056.2\|[30683048] |

| | |
|---|---|
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling X T2 Mature X T3 Seedling |

Bidirectionality: NO Exons: NO Repeats: None Noted
Promoter utility

| | |
|---|---|
| Trait Area: | Among other uses this promoter sequence could be useful to improve: |
| | Water use efficiency - |
| | Nutrient - nitrogen use, Nutrient efficiency |
| | Plant Growth and Development - growth rate |
| Utility: | Useful for root nutrient uptake and metabolism manipulation |

| | |
|---|---|
| Construct: | YP0040 |
| Promoter Candidate I.D: | 11768694 |
| cDNA I.D: | 12670159 (Old ID: 11020088) |
| T1 lines expressing (T2 seed): | SR00588-01, -02, -03 |

Promoter Expression Report # 37
Report Date: January 31, 2003
Promoter Tested In: Arabidopsis thaliana, WS ecotype
Spatial expression summary:

| | |
|---|---|
| Flower | (L)pedicel, (L)stomata |
| Stem | (L)stomata |
| Leaf | (L)vascular, (L)stomata |
| Cotyledon | (H)mesophyll, (H)vascular, (H)epidermis |
| Primary Root | (H)root hairs |

Observed expression pattern: Low GFP expression in stomatal cells of stem, pedicels,
and vasculature of leaves in mature plants. High GFP expression in root hairs, epidermis
and mesophyll cells of seedling cotyledon. Not seen in rosette leaves.
T2 mature: Same as T1 mature expression.
T3 seedling: Same as T2 seedling expression.
Expected expression pattern: Constitutively expressed in all green tissues

| | |
|---|---|
| Selection Criteria: | Arabidopsis microarray |
| Gene: | Expressed protein [Arabidopsis thaliana] |
| GenBank: | NM_119524 Arabidopsis thaliana expressed protein |
| | (At4g33666) mRNA, complete cds |
| | gi\|30689773\|ref\|NM_119524.2\|[30689773] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling XT2 Mature X T3 Seedling |
| Bidirectionality: | Exons: Repeats: |
| Promoter utility | |

| | |
|---|---|
| Trait Area: | Among other uses this promoter sequence could be useful to improve: |
| | PG&D |

| | |
|---|---|
| Sub-trait Area: | Plant size, growth rate, stay green, |

| | |
|---|---|
| Utility: | Useful for C/N partitioning, photosynthetic efficiency, source enhancement and seedling |
| | establishment |

| | |
|---|---|
| Construct: | YP0056 |
| Promoter Candidate I.D: | 11768645 |
| cDNA I.D: 12396394 (Old ID: | 7083850) |

| | |
|---|---|
| T1 lines expressing (T2 seed): | SR00550-01 |

Promoter Expression Report # 38
Report Date: March 24, 2003
Promoter Tested In: Arabidopsis thaliana, WS ecotype
Spatial expression summary:

| | |
|---|---|
| Primary root | (H)root hairs |

Observed expression pattern: GFP expression specific to epidermal root hairs at
hypocotyl root transition zone. This line was not screened in T2 mature and T3 seedlings.
Expected expression pattern: Shoot apical meristem

| | |
|---|---|
| Selection Criteria: | Greater than 5x down in stm microarray |
| Gene: | hypothetical protein |
| GenBank: | NM_118575 Arabidopsis thaliana RNA recognition motif |
| | (RRM)-containing protein (At4g24420) mRNA, complete |
| | cds gi\|18416342\|ref\|NM_118575.1\|[18416342] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling T2 Mature T3 Seedling |
| Bidirectionality: | Exons: Fail Repeats: |
| Promoter utility | |

| | |
|---|---|
| Trait Area: | Among other uses this promoter sequence could be useful to improve: |
| | Water use efficiency; Nutrient |

| | |
|---|---|
| Sub-trait Area: | Plant size, growth rate, drought, water use efficiency, nitrogen |
| | utilization |

| | |
|---|---|
| Utility: | early establishment of Rhizobium infection by increasing expression of elicitors |

| | |
|---|---|
| Construct: | YP0068 |
| Promoter Candidate I.D: | 11768798 |
| cDNA I.D: | 12678173 (Old ID: 1022896) |
| T1 lines expressing (T2 seed): | SR00598-01, -02 |

Promoter Expression Report # 39
Report Date: March 24, 2003
Promoter Tested In: Arabidopsis thaliana, WS ecotype
Spatial expression summary:

| | |
|---|---|
| Primary root | (H)root hairs |

Observed expression pattern: High GFP expression specific to epidermal root hair at
hypocotyls root transition zone. Screened under non-induced condition.
T1 mature: No expression detected.
T2 mature: No expression detected.
T3 seedling: Same expression as T2 seedlings. GFP specific to root hairs.
Expected expression pattern: Heat inducible.
Selection Criteria: Expression data (full_chip) >30 fold induction at 42 C at 1 h and 6

| | |
|---|---|
| Gene: | LMW heat shock protein - mitochondrial |
| GenBank: | NM_118652 Arabidopsis thaliana mitochondrion-localized |
| | small heat shock protein (At4g25200) mRNA, complete cds |
| | gi\|30686795\|ref\|NM_118652.2\|[30686795] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature X T3 Seedling |
| Bidirectionality: NO | Exons: NO Repeats: NO |
| Promoter utility | |

| | |
|---|---|
| Trait Area: | Among other uses this promoter sequence could be useful to improve: |
| | Water use efficiency; Nutrient |

| | |
|---|---|
| Sub-trait Area: | Increase plant growth or seed yield under heat stress conditions, |
| | nitrogen utilization, low N tolerance |
| Utility: | Useful for root nutrient uptake |

| | |
|---|---|
| Construct: | YP0082 |
| Promoter Candidate I.D: | 13148250 (Old ID: 11768604) |
| cDNA I.D: | 13609100 (Old IDs: 12678209, 6462494) |
| T1 lines expressing (T2 seed): | SR00606-01, -02, -03 |

Promoter Expression Report # 40
Report Date: March 24, 2003
Promoter Tested In: Arabidopsis thaliana, WS ecotype
Spatial expression summary:

| | |
|---|---|
| Hypocotyl | (H)epidermis |
| Primary Root | (H)epidermis, (H)trichoblast, (H)root hairs |

Observed expression pattern: High GFP expression throughout epidermal layer of
hypocotyl and upper root including root hairs. Not detected in lower root. No expression
observed in T1 mature plants. T2 mature: No expression observed. T3 seedling: Same
expression as T2 seedlings.

| | |
|---|---|
| Expected expression pattern: | Root |
| Selection Criteria: | Genome annotation |

Gene: ABI3-interacting protein 2 homolog (but recent annotation changed as
hypothetical protein and promoter position is opposite orientation in the hypothetical
protein, see map below); unknown protein

| | |
|---|---|
| GenBank: | NM_101286 Arabidopsis thaliana zinc finger (C3HC4- |
| | type RING finger) protein family (At1g14200) mRNA, |
| | complete cds gi\|30683647\|ref\|NM_101286.2\|[30683647] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature X T3 Seedling |

Bidirectionality: Fail Exons: Fail Repeats: NO
Promoter utility

| | |
|---|---|
| Trait Area: | Among other uses this promoter sequence could be useful to improve: |
| | PG&D |

| | |
|---|---|
| Sub-trait Area: | Nitrogen utilization; plant size, growth rate |
| Utility: | Useful for nutrient uptake e.g., root hairs root epidermis |

| | |
|---|---|
| Construct: | YP0019 |
| Promoter Candidate I.D: | 11768613 |
| cDNA I.D: | 4909291 |
| T1 lines expressing (T2 seed): | SR00489-01, -02 |

Promoter Expression Report # 42
Report Date: March 22, 2003
Promoter Tested In: Arabidopsis thaliana, WS ecotype
Spatial expression summary:

| | |
|---|---|
| Flower | (L)receptacle, (L)vascular |
| Silique | (L)vascular |
| Stem | (L)vascular, (L)phloem |
| Primary root: | (H)phloem |

| | |
|---|---|
| Observed expression pattern: | High GFP expression specific to the seedling root |

phloem tissue. T1 mature: No expression was observed. T2 mature: Low expression in
flower and stem vascular tissues was not observed in T1 mature. T3 seedlings: Same
vascular expression exists as T2 seedlings.
Expected expression pattern: Constitutive in all green tissues

| | |
|---|---|
| Selectin Criteria: | cDNA cluster |
| Gene: | 40S ribosomal protein S5 |
| GenBank: | NM_129283 Arabidopsis thaliana 40S ribosomal protein |
| | S5 (RPS5A) (At2g37270) mRNA, complete cds |
| | gi\|30687090\|ref\|NM_129283.2\|[30687090] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature X T3 Seedling |
| Bidirectionality: NO | Exons: NO Repeats: NO |
| Promoter utility | |

| | |
|---|---|
| Trait Area: | Among other uses this promoter sequence could be useful to improve: |
| | PG&D, Nutrient economy |
| Sub-trait Area: | Plant size, growth rate, low nitrogen tolerance, NUE |
| Utility: | Useful for root nutrient uptake, source/sink relationships, root growth |

| | |
|---|---|
| Construct: | YP0087 |
| Promoter Candidate I.D: | 12748731 |
| cDNA I.D: | 13580795 (Old IDs: 11006078, 12581302) |
| T1 lines expressing (T2 seed): | SR00583-01, -02 |

Promoter Expression Report # 43
Report Date: March 25, 2003
Promoter Tested In: Arabidopsis thaliana, WS ecotype
Spatial expression summary: Screened under non-induced conditions

| | |
|---|---|
| Flower | (H)petal, (H)epidermis, (H)anther |
| Stem | (H)epidermis |
| Cotyledon | (H)epidermis |
| Hypocotyl | (L)epidermis, (L)stomata |
| Rosette Leaf | (L)petiole, (L)stomata |
| Primary Root | (H)phloem, (H)vascular |

Observed expression pattern: T1 mature: High GFP expression in petals of developing
to mature flowers and in and pollen nutritive lipid rich ameboid tapetum cells in
developing anthers. T2 seedling: High GFP expression in root phloem with weak
expression in epidermal tissues of seedlings. T2 mature: Same as T1 mature with
additional stem epidermal expression was not observed in T1 mature plants. T3 seedling:
Same as T2 seedling, however, no expression was seen in epidermal cells of hypocotyls
as in T2 seedlings.
Expected expression pattern: : Inducible promoter - was induced by different forms of
stress (e.g., drought, heat, cold)

| | |
|---|---|
| Selection Criteria | Arabidopsis microarray |
| Gene: | Putative strictosidine synthase |
| GenBank: | NM_147884 Arabidopsis thaliana strictosidine synthase |
| | family (At5g22020) mRNA, complete cds |
| | gi\|30688266\|ref\|NM_147884.2\|[30688266] |
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling XT2 Mature X T3 Seedling |
| Bidirectionality: NO | Exons: FAILS Repeats: N0 |
| Promoter utility | |

| | |
|---|---|
| Trait Area: | PD&G, Nutrient, seed, water use efficiency |
| Sub-trait Area: | Nutrient uptake, C/N partitioning, Source enhancement, source/sink |
| Utility: | Useful for nutrient uptake and transport in root, transport or mobilization |
| | of steroid reserves |

| | |
|---|---|
| Construct: | YP0180 |
| Promoter Candidate I.D: | 11768712 |
| cDNA I.D: | 5787483 (Old IDs: 2918666, 12367001) |
| T1 lines expressing (T2 seed): | SR00902-01, -02, -03 |

Promoter Expression Report # 44
Report Date: March 22, 2003
Promoter Tested In: Arabidopsis thaliana, WS ecotype
Spatial expression summary:

| | |
|---|---|
| Hypocotyl | (L)epidermis |

Observed expression pattern: Low GFP expression in the epidermal cells of hypocotyl.
Screened under non-induced conditions. No T1 mature expression was observed. T2
mature: No expression was observed. T3 seedling: Same expression as the T2 seedling
seen in one of two events. Guard cell expression was observed in second event.
Expected expression pattern: Induced by different forms of stress (e.g., drought, heat,
cold).

| | |
|---|---|
| Selection Criteria: | Arabidopsis microarray. Induced by different forms of |
| | stress (e.g., drought, heat, cold) |
| Gene: | Berberine bridge enzyme |
| GenBank: | NM_100078 Arabidopsis thaliana FAD-linked |
| | oxidoreductase family (At1g01980) mRNA, complete cds |
| | gi\|18378905\|ref\|NM_100078.1\|[18378905] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature X T3 Seedling |
| Bidirectionality: NO | Exons: NO Repeats: NO |
| Promoter utility | |

| | |
|---|---|
| Trait Area: | Among other uses this promoter sequence could be useful to improve: |
| | Water use efficiency; PG&D |
| Sub-trait Area: | Heat |
| Utility: | Seedling establishment, |

| | |
|---|---|
| Construct: | YP0186 |
| Promoter Candidate I.D: | 11768854 |
| cDNA I.D: | 13647840 (Old IDs: 12689527, 11437778) |
| T1 lines expressing (T2 seed): | SR00906-02, -03 |

Promoter Expression Report # 45
Report Date: March 25, 2003
Promoter Tested In: Arabidopsis thaliana, WS ecotype
Spatial expression summary:

| | |
|---|---|
| Ovule | Pre-fertilization: (H)inner integument |
| | Post-fertilization: (H)inner integument, (H)outer integument |

Observed expression pattern: High GFP expression specific to the inner integuments
of developing pre-fertilized ovules and outer integuments at the mycropylar end of post
fertilized ovules. GFP detected throughout inner integument of developing seed at
mature embryo stage. T2 seedling: No expression observed. T2 Mature: Same
expression as observed in T1 mature. T3 seedling: Not screened.

| | |
|---|---|
| Expected expression pattern: | Expressed in ovules and different parts of seeds |
| Selection Criteria: | Greater than 50x up in pi ovule microarray |
| Gene: | pectin methylesterase [Arabidopsis thaliana]. |
| GenBank: | NM_124295 Arabidopsis thaliana pectinesterase family |
| | (At5g49180) mRNA, complete cds |
| | gi\|30695612\|ref\|NM_124295.2\|[30695612] |
| Source Promoter Organism: | Arabidopsis thaliana WS |
| Vector: | pNewBin4-HAP1-GFP |
| Marker Type: | X GFP-ER |
| Generation Screened: | X T1 Mature X T2 Seedling X T2 Mature T3 Seedling |

Bidirectionality: NO Exons: FAILS Repeats: NO
Promoter utility

| | |
|---|---|
| Trait Area: | Seed, Yield, Nutrient, cold, water use efficiency |
| Sub-trait Area: | Ovule/seed abortion, seed enhamcement, seed number, seed size, |
| | total yield, seed nitrogen, cold germination and vigor |
| Utility: | Useful for improvement for seed yield, composition, moisture |
| | stress at seed set, moisute stress during seed fill |

| | |
|---|---|
| Construct: | YP0121 |
| Promoter Candidate I.D: | 11768686 |
| cDNA I.D: | 12646933 (Old IDs: 12370661, 7080188) |
| T1 lines expressing (T2 seed): | SR00805-01, -02, -03 |

Promoter Expression Report # 46
Report Date: March 25, 2003
Promoter Tested In: Arabidopsis thaliana, WS ecotype
Spatial expression summary:

| Silique | (H)ovule | |
|---|---|---|
| Ovule | Pre-fertilization: | (H)embryo sac, (H)gametophyte |
| | Post-fertilization: | (H)zygote |

Observed expression pattern: GFP expression is specific to female gametophyte and
surrounding sporophytic tissue of pre-fertilized ovules and zygote of fertilized ovule 0-5
hours after fertilization (HAF). Not detected in developing embryos. T2 mature: Did not
germinate.
T3 seedlings: No seeds available.

| | |
|---|---|
| Expected expression pattern: | Expressed in ovules and different parts of seeds |
| Selection Criteria: | Greater than 50x up in pi ovule microarray |
| Gene: | hypothetical protein |
| GenBank: | NM_123661 Arabidopsis thaliana expressed protein |
| | (At5g42955) mRNA, complete cds |
| | gi\|18422274\|ref\|NM_123661.1\|[18422274] |
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling T2 Mature T3 Seedling |

Bidirectionality: NO Exons: NO Repeats: NO
Promoter utility

| | |
|---|---|
| Trait Area: | Among other uses this promoter sequence could be useful to |
| | improve: |
| | Seed, yield, quality |
| Sub-trait Area: | Ovule/seed abortion, harvest index, test weight, seed size, total |
| | yield, amino acid, protein, total oil, total seed composition |
| Utility: | This is promoter is useful for enhance of seed composition, seed size, |
| | seed number and yield, etc. |

| | |
|---|---|
| Construct: | YP0096 |
| Promoter Candidate I.D: | 13148242 (Old ID: 11768682) |
| cDNA I.D: | 4949423 (Old IDs: 12325608, 1007532) |
| T1 lines expressing (T2 seed): | SR00775-01, -02 |

Promoter Expression Report # 47
Report Date: March 25, 2003
Promoter Tested In: Arabidopsis thaliana, WS ecotype
Spatial expression summary:

| | |
|---|---|
| Flower | (H)pedicel, (H)stomata |
| Silique | (M)stomata |
| Stem | (M)stomata |
| Rosette Leaf | (L)stomata |
| Primary Root | (H)root hairs |

Observed expression pattern: Guard cell expression throughout stem, pedicels, and
siliques.
High GFP preferential expression to root hairs of seedlings and medium to low
expression in primary rosette leaves and petioles and stems.
T2 mature: Same expression as T1 mature.
T3 seedlings: Same expression as T2 seedlings.
Expected expression pattern: Expressed in ovules and different parts of seeds

| | |
|---|---|
| Selection Criteria: | Greater than 50x up in pi ovule microarray |
| Gene: | hypothetical protein |
| GenBank: | NM_122878 Arabidopsis thaliana expressed protein |
| | (At5g34885) mRNA, complete cds |
| | gi\|30692647\|ref\|NM_122878.2\|[30692647] |
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling X T2 Mature X T3 Seedling |

Bidirectionality: NO Exons: NO Repeats: NO
Promoter utility

| | |
|---|---|
| Trait Area: | Among other uses this promoter sequence could be useful to improve: |
| | Water use efficiency, PG&D, nutrient |
| Sub-trait Area: | Drought, heat, water use efficiency, plant size, low nitrogen utilization |
| Utility: | Useful for root nutrient uptake, plant growth under drought, heat |

| | |
|---|---|
| Construct: | YP0098 |
| Promoter Candidate I.D: | 12758479 |
| cDNA I.D: 4906343 (Old IDs: | 12662283, 1024001) |
| T1 lines expressing (T2 seed): | SR00896-01, -02 |

Promoter Expression Report # 48
Report Date: March 25, 2003
Promoter Tested In: Arabidopsis thaliana, WS ecotype
Spatial expression summary:

| | |
|---|---|
| Flower | (H)pedicel, (H)sepal, (H)vascular |
| Silique | (H)septum, (H)vascular |
| Stem | (H)vascular |
| Leaf | (H)petiole, (H)vascular, (H)phloem |
| Hypocotyl | (H)vascular |
| Primary Root | (H)vascular, (H)phloem |

Observed expression pattern: High GFP expression throughout mature and seedling
vascular tissue. T2 mature and T3 seedling: Not screened.

| | |
|---|---|
| Expected expression pattern: | Expressed in ovules and different parts of seeds |
| Selection Criteria: | Greater than 50x up in pi ovule microarray |
| Gene: | unknown protein; expressed protein |
| GenBank: | NM_129068 Arabidopsis thaliana expressed protein |
| | (At2g35150) mRNA, complete cds |
| | gi\|30686319\|ref\|NM_129068.2\|[30686319] |

Source Promoter Organism: Arabidopsis thaliana WS

| | |
|---|---|
| Vector: | pNewBin4-HAP1-GFP |
| Marker Type: | X GFP-ER |
| Generation Screened: | XT1 Mature X T2 Seedling T2 Mature T3 Seedling |

Bidirectionality: NO Exons: FAILS Repeats: NO
Promoter utility

| | |
|---|---|
| Trait Area: | Among other uses this promoter sequence could be useful to |
| | improve: |
| | PG&D, nutrient, seed |
| Sub-trait Area: | Growth rate, plant size, low nitrogen use efficiency, nitrogen utilization, |
| | seed size and yield |

Utility: Useful for root nutrient uptake and transport, enhance plant growth rate under low
nitrogen condition. Enhance plant to use water efficiently. Might be also useful for seed program.
Source/sink

| | |
|---|---|
| Construct: | YP0108 |
| Promoter Candidate I.D: | 11768683 |
| cDNA I.D: 13601936 (Old IDs: | 12339941, 4768517) |
| T1 lines expressing (T2 seed): | SR00778-01, -02 |

Promoter Expression Report # 49
Report Date: March 25, 2003
Promoter Tested In: Arabidopsis thaliana, WS ecotype
Spatial expression summary: Screened under non-induced conditions.

| | |
|---|---|
| Flower | (H)septum, (H)epidermis |
| Silique | (L)carpel, (H)septum, (H)epidermis, (M)vascular |
| Stem | (M)epidermis |
| Hypocotyl | (L)epidermis, (L)stomata |
| Cotyledon | (L)epidermis, (L)guard cell |
| Primary Root | (H)epidermis, (H)trichoblast, (H)atrichoblast, (H)root hairs |

Observed expression pattern: High preferential GFP expression in septum epidermal
cells in siliques and root hair cells of seedlings. Low expression in cotyledon and
hypocotyl epidermal cells. T2 mature: Stem epidermal and silique vascular expression
observed in addition to expression observed in T1 mature. Expression in stem epidermal
cells appears variable. T3 seedling: Same expression as T2 seedlings with additional
guard cell expression in siliques.
Expected expression pattern: Root

| | |
|---|---|
| Selection Criteria: | Greater than 10x induced by Roundup. Induced in |
| | Arabidopsis microarray at 4 hours |
| Gene: | Hypothetical protein |
| GenBank: | NM_111930 Arabidopsis thaliana expressed protein |
| | (At3g10930) mRNA, complete cds |
| | gi\|30681550\|ref\|NM_111930.2\|[30681550] |

Source Promoter Organism: Arabidopsis thaliana WS

| | |
|---|---|
| Vector: | pNewBin4-HAP1-GFP |
| Marker Type: | X GFP-ER |
| Generation Screened: | XT1 Mature X T2 Seedling X T2 Mature X T3 Seedling |

Bidirectionality: NO Exons: NO Repeats: NO
Promoter utility

| | |
|---|---|
| Trait Area: | Among other uses this promoter sequence could be useful to |
| | improve: |
| | Water use efficiency, PG&D, nutrient, yield |
| Sub-trait Area: | Drought, growth rate, plant size, low nitrogen use efficiency, nitrogen |
| | utilization; seed yield |
| Utility: | Useful for root nutrient uptake, enhance plant growth rate under low |
| | nitrogen condition. Enhance plant to use water efficiency, useful for pod |
| | shatter |

| | |
|---|---|
| Construct: | YP0134 |
| Promoter Candidate I.D: | 11768684 |
| cDNA I.D: | 13489977 (Old IDs: 12332605, 6403797) |
| T1 lines expressing (T2 seed): | SR00780-02, -03 |

Promoter Expression Report # 50
Report Date: March 25, 2003
Promoter Tested In: Arabidopsis thaliana, WS ecotype
Spatial expression summary: Screened under non-induced conditions

| | |
|---|---|
| Flower | (H)pedicel, (L)petal, (H)silique |
| Silique | (H)carpel, (H)cortex, (H)epidermis |
| Ovule | Post-fertilization: (L)outer integument |
| Embryo | (L)mature |
| Stem | (M)epidermis, (H)cortex, (H)endodermis |
| Leaf | (H)petiole, (H)mesophyll, (H)epidermis |
| Cotyledon | (H)mesophyll, (H)epidermis |
| Rosette Leaf | (H)mesophyll, (L)vascular, (H)epidermis |
| Primary Root | (H)cortex |
| Lateral root | (H)cortex, (H)flanking cells |

Observed expression pattern: High preferential GFP expression in photosynthetic,
cortical and epidermal tissues in mature plants and seedlings. T2 mature: Weak outer
integument expression in mature ovules and mature embryo in addition to expression
observed in T1 mature plants. T3 seedling: Same expression observed as T2 seedlings
(seen in one event). Weak epidermal and high lateral root flanking cell expression
observed in second event.
Expected expression pattern: Root hairs

| | |
|---|---|
| Selection Criteria: | Ceres Microarray 2.5-5X down in rhl (root hair less) |
| | mutant |
| Gene: | probable auxin-induced protein |
| GenBank: | NM_119642 Arabidopsis thaliana auxin-induced (indole-3- |
| | acetic acid induced) protein family (At4g34760) mRNA, |
| | complete cds gi\|30690121\|ref\|NM_119642.2\|[30690121] |

Source Promoter Organism: Arabidopsis thaliana WS

| | |
|---|---|
| Vector: | pNewBin4-HAP1-GFP |
| Marker Type: | X GFP-ER |
| Generation Screened: | XT1 Mature X T2 Seedling X T2 Mature X T3 Seedling |

Bidirectionality: NO Exons: NO Repeats: NO
Promoter utility

| | |
|---|---|
| Trait Area: | Among other uses this promoter sequence could be useful to |
| | improve: |
| | PG&D, Nutrient; C3-C4 optimization |
| Sub-trait Area: | Low nitrogen use efficiency, nitrogen utilization, low nitrogen |
| | tolerance, plant size, growth rate, water use efficiency; manipulate |
| | expression of C3-C4 enzymes in leaves |
| Utility: | Useful for root nutrient uptake and transport, enhance plant growth rate, |
| | also for enhance of plant water use efficency |

| | |
|---|---|
| Construct: | YP0138 |
| Promoter Candidate I.D: | 13148247 (Old ID: 11768685) |
| cDNA I.D: | 12333534 (Old ID: 7077536) |
| T1 lines expressing (T2 seed): | SR00781-01, -02, -03 |

Promoter Expression Report # 52
Report Date: March 25, 2003
Promoter Tested In: Arabidopsis thaliana, WS ecotype
Spatial expression summary:

| | |
|---|---|
| Flower | (L)sepal, (L)vascular |
| Rosette Leaf | (L)vascular, (L)stomata |

Observed expression pattern: Weak GFP expression in sepal vasculature of developing
flower buds. Weak expression in vasculature and guard cells of rosette leaves. Not
detected in mature flowers. T2 mature: Same expression as T1 mature detected in one of
two events. Vascular expression in pedicels of developing flowers. T3 seedlings: No
expression detected.
Expected expression pattern: Shoot apex including leaf primordia and parts of leaves

| | |
|---|---|
| Selection Criteria: | Greater than 5x up in stm microarray |
| Gene: | unknown protein |
| GenBank: | NM_122151 Arabidopsis thaliana |
| | esterase/lipase/thioesterase family (At5g22460) mRNA, complete cds |
| | gi\|30688485\|ref\|NM_122151.2\|[30688485] |
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling X T2 Mature X T3 Seedling |
| Bidirectionality: NO | Exons: FAILS Repeats: NO |

| | |
|---|---|
| Promoter utility | |
| Trait Area: | Among other uses this promoter sequence could be useful to improve: |
| | Water use efficiency |
| Sub-trait Area: | Water use efficiency |
| Utility: | This is weak promoter expressed in guard cell and flower. Might be |
| | useful for water use efficiency |

| | |
|---|---|
| Construct: | YP0192 |
| Promoter Candidate ID: | 11768715 |
| cDNA I.D: 12688453 (Old IDs: | 12384618, 3434328) |
| T1 lines expressing (T2 seed): | SR00908-01, -02 |

Promoter Expression Report # 53
Report Date: March 25, 2003
Promoter Tested In: Arabidopsis thaliana, WS ecotype
Spatial expression summary:

| | |
|---|---|
| Flower | (H)pedicel, (H)vascular |
| Primary Root | (H)epidermis, (H)trichoblast, (H)atrichoblast, (L)root hair |

Observed expression pattern: High GFP expression specific in floral pedicel vascular
tissue of developing flowers. Not detected in pedicels and stems of mature plants. High
GFP expression throughout epidermal layers of primary seedling root. T2 mature: No
expression in 3 plants observed. T3 seedling: Same as T2 seedling expression.

| | |
|---|---|
| Expected expression pattern: | Inducible promoter - induced by different forms of stress |
| | (e.g., drought, heat, cold). |
| Selection Criteria: | Arabidopsis microarray |
| Gene: | Reticuline oxidase; berberine bridge enzyme |
| GenBank: | NM_102806 Arabidopsis thaliana FAD-linked |
| | oxidoreductase family (At1g30700) mRNA, complete cds |
| | gi\|30692021\|ref\|NM_102806.2\|[30692021] |
| Source Promoter Organism: | Arabidopsis thaliana WS |
| Vector: | pNewBin4-HAP1-GFP |
| Marker Type: | X GFP-ER |
| Generation Screened: | XT1 Mature X T2 Seedling X T2 Mature X T3 Seedling |
| Bidirectionality: NO | Exons: NO Repeats: NO |

| | |
|---|---|
| Promoter utility | |
| Trait Area: | PG&D, Nutrient. Seed development, yield |
| Sub-trait Area: | Plant size, growth rate, nitrogen use efficiency and utilization |
| Utility: | Very useful for root nutrient uptake, enhancement for plant growth |
| | under low nitrogen condition |

| | |
|---|---|
| Construct: | YP0204 |
| Promoter Candidate I.D: | 11768721 |
| cDNA I.D: | 12669615 (Old ID: 7089815) |
| T1 lines expressing (T2 seed): | SR00914-01, -03, -04 |

Promoter Expression Report # 54
Report Date: March 31, 2003

| | |
|---|---|
| Promoter Tested In: | I. Arabidopsis thaliana, WS ecotype |
| | II. Oryza sativa |
| | III. Lycopersicon esculentum. |

Spatial expression summary:
I. Arabidopsis thaliana

| | |
|---|---|
| Flower | (H)pedicel, (H)receptacle, (H)nectary, (H)sepal, (H)petal, |
| | (H)filament, (H)anther, (H)carpel, (H)style, (H)stigma, |
| | (H)epidermis |
| Silique | (H)stigma, (H)style, (H)carpel, (H)septum, (H)placentae, |
| | (H)epidermis, (H)ovule |
| Ovule | Pre-fertilization: (H)inner integument, (H)outer integument, |
| | (H)embryo sac, (H)funiculus, (H)chalaza, (H)micropyle |
| | Post-fertilization: (H)inner integument, (H)outer integument, |
| | (H)seed coat, (H)chalaza, (H)micropyle, (H)embryo |
| Embryo | (H)late, (H)mature |
| Stem | (H)epidermis, (H)cortex, (H)vascular |
| Leaf | (H)petiole, (H)mesophyll, (H)epidermis |
| Hypocotyl | (M)epidermis |
| Cotyledon | (H)mesophyll, (H)epidermis |
| Primary Root | (H)epidermis, (H)atrichoblas, (H)vascular, (H)cap |
| Lateral root | (H)epidermis, (H)initials, (H)cap |
| II. Oryza sativa | |
| Leaf sheath | epidermis, vascular, cortex |
| Leaf | mesophyll, vascular |
| Lateral root | initials, cap |
| Primary root | cap |
| Embryo | 5 day |

III. Lycopersicon esculentum

| | |
|---|---|
| Leaf | mesophyll |
| Flower | ovules, stamen, pollen |
| Root | epidermis |
| Fruit | peel tissue |

Observed expression patterns: T2 mature and T2 seedling: Expressed throughout mature
and seedling tissues. High expression in L1, L2, and L3 layers of shoot apical meristem.

| | |
|---|---|
| Expected expression pattern: | Constitutive |
| Selection Criteria: | cDNA cluster |
| Gene: | Arabidopsis Elongation Factor 1-α |
| GenBank: | NM_125432 Arabidopsis thaliana elongation factor |
| | 1-alpha (EF-1-alpha) (At5g60390) mRNA, complete cds |
| | gi\|30697365\|ref\|NM_125432.2\|[30697365] |
| Source Promoter Organism: | Arabidopsis thaliana WS |
| Vector: | CRS-BIN2A2 |
| Marker Type: | Histone-YFP |

| | | | | |
|---|---|---|---|---|
| Generation Screened: | | | | |
| I. Arabidopsis thaliana | □ T1 Mature | X T2 Seedling | X T2 Mature | □T3 Seedling |
| II. Oryza sativa | X T1 Mature | □ T2 Seedling | □ T2 Mature | □T3 Seedling |
| III. Lycopersicon esculentum | X T1 Mature | □ T2 Seedling | □ T2 Mature | □T3 Seedling |

Criteria: Bidirectionality: NO Exons: NO Repeats: NO

| | |
|---|---|
| Trait Area: | Among other uses, this promoter sequence could be useful to improve: |
| | Water use efficiency, PG&D, Seed, Nutrient, Yield |

| | |
|---|---|
| Construct: | BIN2A2/28716-HY2 |
| Promoter Candidate I.D: | 12786308 |
| cDNA I.D: | 12739224 (Old ID: 12731344) |

Promoter Expression Report # 54
Report Date: March 31, 2003

| | |
|---|---|
| Promoter Tested In: | I. Arabidopsis thaliana, WS ecotype |
| | II. Oryza sativa |
| | III. Lycopersicon esculentum. |

Spatial expression summary:
I. Arabidopsis thaliana

| | |
|---|---|
| Flower | (H)pedicel, (H)receptacle, (H)nectary, (H)sepal, (H)petal, |
| | (H)filament, (H)anther, (H)carpel, (H)style, (H)stigma, |
| | (H)epidermis |
| Silique | (H)stigma, (H)style, (H)carpel, (H)septum, (H)placentae, |
| | (H)epidermis, (H)ovule |
| Ovule | Pre-fertilization: (H)inner integument, (H)outer integument, |
| | (H)embryo sac, (H)funiculus, (H)chalaza, (H)micropyle |
| | Post-fertilization: (H)inner integument, (H)outer integument, |
| | (H)seed coat, (H)chalaza, (H)micropyle, (H)embryo |
| Embryo | (H)late, (H)mature |
| Stem | (H)epidermis, (H)cortex, (H)vascular |
| Leaf | (H)petiole, (H)mesophyll, (H)epidermis |
| Hypocotyl | (M)epidermis |
| Cotyledon | (H)mesophyll, (H)epidermis |
| Primary Root | (H)epidermis, (H)atrichoblas, (H)vascular, (H)cap |
| Lateral root | (H)epidermis, (H)initials, (H)cap |
| II. Oryza sativa | |
| Leaf sheath | epidermis, vascular, cortex |
| Leaf | mesophyll, vascular |
| Lateral root | initials, cap |
| Primary root | cap |
| Embryo | 5 day |

III. Lycopersicon esculentum

| | |
|---|---|
| Leaf | mesophyll |
| Flower | ovules, stamen, pollen |
| Root | epidermis |
| Fruit | peel tissue |

Observed expression patterns: T2 mature and T2 seedling: Expressed throughout mature
and seedling tissues. High expression in L1, L2, and L3 layers of shoot apical meristem.

| | |
|---|---|
| Expected expression pattern: | Constitutive |
| Selection Criteria: | cDNA cluster |
| Gene: | Arabidopsis Elongation Factor 1-α |
| GenBank: | NM_125432 Arabidopsis thaliana elongation factor |
| | 1-alpha (EF-1-alpha) (At5g60390) mRNA, complete cds |
| | gi\|30697365\|ref\|NM_125432.2\|[30697365] |
| Source Promoter Organism: | Arabidopsis thaliana WS |
| Vector: | CRS-BIN2A2 |
| Marker Type: | Histone-YFP |

| | | | | |
|---|---|---|---|---|
| Generation Screened: | | | | |
| I. Arabidopsis thaliana | □ T1 Mature | X T2 Seedling | X T2 Mature | □T3 Seedling |
| II. Oryza sativa | X T1 Mature | □ T2 Seedling | □ T2 Mature | □T3 Seedling |
| III. Lycopersicon esculentum | X T1 Mature | □ T2 Seedling | □ T2 Mature | □T3 Seedling |

Criteria: Bidirectionality: NO Exons: NO Repeats: NO
Promoter utility

| | |
|---|---|
| Trait Area: | Among other uses, this promoter sequence could be useful to improve: |
| | Water use efficiency, PG&D, Seed, Nutrient, Yield |

| | |
|---|---|
| Construct: | BIN2A2/28716-HY2 |
| Promoter Candidate I.D: | 12786308 |
| cDNA I.D: | 12739224 (Old ID: 12731344) |

Promoter Expression Report # 55
Report Date: March 23, 2003

| | |
|---|---|
| Promoter Tested In: | I. Arabidopsis thaliana, WS ecotype |
| | II. Oryza sativa |

Spatial expression summary:
I. Arabidopsis thaliana, WS ecotype

| | |
|---|---|
| Flower | (H)pedicel, (H)receptacle, (H)nectary, (H)sepal, (H)petal, |
| | (H)filament, |
| | (H)anther, (H)pollen, (H)carpel, (H)style, (H)papillae, |
| | (H)epidermis, |
| | (H)SAM |
| Silique | (H)stigma, (H)style, (H)carpel, (H)septum, (H)placentae, |
| | (H)transmitting (H)tissue, (H)epidermis, (H)ovule |
| Ovule | Pre-fertilization: (H)inner integument, (H)outer integument, |
| | (H)embryo sac, (H)funiculus, (H)chalaza, (H)micropyle |
| | Post-fertilization: (H)zygote, (H)inner integument, (H)outer |
| | integument, |
| | (H)seed coat, (H)chalaza, (H)micropyle, (H)early endosperm, |
| | (H)mature endosperm, (H)embryo |
| Embryo | (H)suspensor, (H)preglobular, (H)globular, (H)heart, (H)torpedo, |
| | (H)late, (H)mature, (H)hypophysis, (H)radicle, (H)cotyledons, |
| | (H)hypocotyl |
| Stem | (H)epidermis, (H)cortex, (H)vascular, (H)pith |
| Leaf | (H)petiole, (H)mesophyll, (H)epidermis |
| Hypocotyl | (L)epidermis, (L)cortex, (L)vascular |
| Rosette Leaf | (H)mesophyll, (H)epidermis, (H)petiole |
| Primary Root | (H)epidermis, (H)trichoblast, (H)atrichoblast, (H)cortex, (H)cap, |
| | (H)root hairs |
| Lateral Root | (H)epidermis, (H)initials, (H)cap |
| II. Oryza sativa | |
| Flower | |
| Pollen | |
| Leaf sheath | |

| | |
|---|---|
| Observed expression patterns: | Constitutive. Expression observed throughout mature |
| | and seedling plants. |
| Expected expression pattern: | Constitutive |
| Selection Criteria: | cDNA cluster |
| Gene: | Arabidopsis ADP-Ribosylation Factor 1 |
| GenBank: | NM_130285 Arabidopsis thaliana ADP- |
| | ribosylation factor 1 (ARF1) (At2g47170) mRNA, |
| | complete cds |
| | gi\|18407284\|ref\|NM_130285.1\|[18407284] |
| Source Promoter Organism: | Arabidopsis thaliana WS |
| Vector: | CRS-Bin1A1 |
| Marker Type: | X Histone-YFP |

| | | | | |
|---|---|---|---|---|
| Generation Screened: | | | | |
| I. Arabidopsis thaliana | □ T1 Mature | X T2 Seedling | X T2 Mature | □T3 Seedling |
| II. Oryza sativa | X T1 Mature | □ T2 Seedling | □ T2 Mature | □T3 Seedling |

| | |
|---|---|
| Bidirectionality: NO | Exons: NO Repeats: NO |

| | |
|---|---|
| Promoter utility | |
| Trait Area: | Among other uses, this promoter sequence could be useful to improve: |
| | Water use efficiency, PG&D, Seed, Nutrient, Yield |

| | |
|---|---|
| Construct: | BINA1-34414-HY2 |
| Promoter Candidate I.D: | 12786307 |
| cDNA I.D: | 13609583 (Old ID: 12394813) |

Promoter Expression Report # 56
Report Date: March 23, 2003

| | |
|---|---|
| Promoter Tested In: | I. Arabidopsis thaliana, WS ecotype |
| | II. Oryza sativa |

Spatial expression summary:
I. Arabidopsis thaliana

| | |
|---|---|
| Flower | (H)pedicel, (H)receptacle, (H)nectary, (H)sepal, (H)anther, |
| | (H)phloem, |
| | (H)cap, (H)root hairs, (H)pollen, (H)carpel, (H)style, (H)epidermis |
| Silique | (H)style, (H)carpel, (H)septum, (H)placentae, (H)vascular, |
| | (H)epidermis, |
| | (H)ovule |
| Ovule | Pre-fertilization: (H)outer integument, (H)funiculus |
| | Post-fertilization: (H)outer integument, (H)seed coat |
| Stem | (H)epidermis, (H)cortex, (H)vascular, (H)xylem, (H)phloem, |
| | (H)pith |
| Leaf | (M)mesophyll, (H)vascular |
| Hypocotyl | (H)epidermis, (H)vascular |
| Cotyledon | (H)mesophyll, (H)epidermis |
| Primary Root | (H)epidermis, (H)trichoblast, (H)atrichoblast, (H)vascular, (H)xylem, |
| | (H)phloem, (H)cap, (H)root hairs |
| II. Oryza sativa | |
| Flower | (L)vascular |
| Sheath | (H)all cells |
| Leaf tip | (H)all cells |
| Leaf lower blade | (H)vascular |
| Root | (M)vascular, (L)epidermis |
| Lateral root | (H)epidermis |
| Ovule | (H)all structures |
| Immature seed | (M)connective tissue |

Observed expression patterns:
I. Arabidopsis thaliana: Expressed throughout most mature tissues screened. Not
detected in shoot apical meristem and stage 1 and 2 flower buds. Not detected in stamen
and siliques of stage 4 flowers. Not detected in the stigma, which has abnormal
development. Aborted embryos. Not detected in developing embryos. High Expression
in epidermal, vascular and photosynthetic tissue of seedling. Lines characterized have
gone through several generations. Not screened in successive generation.
II. Oryza sativa: High expression throughout leaf sheath, leaf, root, lateral root tip, anther
filament, ovule, stem and connection point between seed and pedicel. Not detectable in
developing seeds. Not expressed in organs of developing flowers.
Expected expression pattern: Constitutive expression

| | |
|---|---|
| Selection Criteria: | From Ceres, Inc. and Stanford microarray data. Selected for |
| | constitutive expression. |
| Gene: | S-Adenosylmethionine Synthetase 2 |
| GenBank: | NM_112618 Arabidopsis thaliana s-adenosylmethionine |
| | synthetase - related (At3g17390) mRNA, complete cds |
| | gi\|30684501\|ref\|NM_112618.2\|[30684501] |

Source Promoter Organism: Arabidopsis thaliana WS

| | |
|---|---|
| Vector: | I. Arabidopsis- CRS-HT1 (Construct: CR13-GFP-ER) |
| | II. Oryza sativa- CRS-HT1 (Construct: CR13-GFP-ER), |
| | CRS-BIN1A (Construct: CR14-hYFP) |
| Marker Type: | I. Arabidopsis- GFP-ER |
| | II. Oryza sativa- GFP-ER, hYFP |

Generation Screened:

| | |
|---|---|
| I. Arabidopsis- | □ T1 Mature X T2 Seedling X T2 Mature □ T3 Seedling |
| II. Oryza sativa- | X T1 Mature X T2 Seedling □ T2 Mature □ T3 Seedling |
| Bidirectionality: | FAILS?? Exons: FAILS?? Repeats: NO |
| Promoter utility | |
| Trait Area: | Among other uses this, promoter sequence could be useful to improve: |
| | Water use efficiency, PG&D, seeds; nutrients |
| Sub-trait Area: | Drought, water use efficiency, growth rate, plant size, low nitrogen |
| | tolerance, nitrogen use efficiency, seed enhancement |
| Utility: | Useful for root nutrient uptake and transport, water use efficiency, |
| | and improvement of seed size, yield, etc. |
| Construct: | CR13 (GFP-ER) |
| | CR14 (H-YFP) |
| Promoter I.D: | 12786306 |
| cDNA I.D: | 13614841 (Old ID: 12331556) |

Promoter Expression Report # 98
Report Date: December 3, 2003
Promoter Tested In: Arabidopsis thaliana, WS ecotype
Spatial expression summary:

| | |
|---|---|
| Flower | H pedicel, H receptacle, H sepal, H epidermis, H endodermis |
| Silique | H placenta |
| Stem | H endodermis |
| Leaf | H endodermis |
| Hypocotyl | M epidermis, L vascular |
| Cotyledon | L vascular |
| Rosette Leaf | H vascular, H epidermis, H mid rib |
| Primary Root | H pericycle, H endodermis, L root hairs |
| Lateral root | H initials |

Observed expression pattern:
T1 mature: Strong GFP expression in rib vein support tissue in flowers, leaves and
endodermis of stems. Appears not to be expressed within vascular tissue.
T2 seedling: Expressed throughout epidermal and vascular tissues of seedling. Expressed
in both mid-vein ground tissue and vasculature of developing leaves. Expression in
ground tissues of roots. Not observed in root vascular.

| | |
|---|---|
| Expected expression pattern: | Shoot meristem |
| Selection Criteria: | Arabidopsis public |
| Gene: | Xyloglucan endotransglycosylase. |
| GenBank: | NM_113277 Arabidopsis thaliana xyloglucan |
| | endotransglycosylase, putative (At3g23730) mRNA, complete cds |
| | gi\|18403866\|ref\|NM_113277.1\|[18403866] |

Source Promoter Organism: Arabidopsis thaliana WS

| | |
|---|---|
| Vector: | pNewbin4-HAP1-GFP |
| Marker Type: | GFP-ER |
| Generation Screened: | X T1 Mature X T2 Seedling □T2 Mature □T3 Seedling |
| Criteria: | Bidirectional: PASS Exons: PASS Repeats: PASS |

Table 3. Promoter utility
Utility: Translocation, seed fill. Improved loading of phloem, increased source capacity. Increased seed
yield.
Notes: The polysaccharide xyloglucan is thought to play an important structural role in the
primary cell wall of dicotyledons. Endodermis: Recent studies have implicated these cell types
in gravity perception by sedimentation of starch within these cells. Gravity perception by dicot
organs involves primarily the sedimentation of amyloplasts within specialized cells (statocytes)
located in the columella region of the root cap and in the starch sheath, which constitutes the
endodermis of hypocotyls and inflorescence stems (Kiss et al., 1996; Kuznetsov and Hasenstein,
1996; Blancaflor et al., 1998; Weise et al., 2000). In shoots, sedimentable amyloplasts and the
curvature response to gravistimulation occur along the elongation zone (for review, see Masson et
al., 2002). After amyloplast sedimentation, signals are likely transduced within the endodermal
cells, and physiological signals are transported laterally to affect elongation of cortical and
epidermal cells. In roots, sites of gravity perception and curvature response may be physically
separated (Poff and Martin, 1989).

| | |
|---|---|
| Construct: | YP0018 |
| Promoter candidate I.D: | 11768673 |
| cDNA I.D: | 12647555 |
| Lines expressing: | YP0018-01; YP0018-02 plant date 7/28/03 |

Promoter Expression Report # 99
Report Date: December 3, 2003
Promoter Tested In: Arabidopsis thaliana, WS ecotype
Spatial expression summary:

| | |
|---|---|
| Flower | L pedicel, L receptacle, L sepal, L petal, L filament, L epidermis |
| Stem | L vascular |
| Leaf | M vascular, L rib |
| Hypocotyl | L epidermis, L cortex, H vascular |
| Cotyledon | L mesophyll, L epidermis |
| Rosette Leaf | L mesophyll, L vascular, L epidermis, H petiole |
| Primary Root | H vascular |

Observed expression pattern:
T1 mature: Weak vascular expression throughout inflorescence meristem and flowers.
Variable levels of expression in cells at receptacle of flowers. Expressed in both vascular
and supporting ground tissue in leaves. T2 seedling: Strong expression observed
throughout vasculature of root and hypocotyl. Expression in a few epidermal and cortex
cells of hypocotyl at cotyledon junction. Weak epidermis and mesophyll expression in
developing leaves.
Expected expression pattern: Stem cell population in center of shoot apical,
inflorescence and floral meristem.

| | |
|---|---|
| Selection Criteria: | Arabidopsis public. Clark SE, Williams RW, Meyerowitz EM. The |
| | CLAVATA1 gene encodes a putative receptor |
| | kinase that controls shoot and floral meristem size |
| | in Arabidopsis. Cell. 1997 May 16; 89(4): 575-85. |
| Gene: | CLAVATA1 receptor kinase (CLV1) |
| GenBank: | NM_106232 Arabidopsis thaliana CLAVATA1 receptor kinase (CLV1) |
| | (At1g75820) mRNA, complete cds gi\|30699119\|ref\|NM_106232.2\|[30699119] |

Source Promoter Organism: Arabidopsis thaliana WS

| | |
|---|---|
| Vector: | pNewbin4-HAP1-GFP |
| Marker Type: | GFP-ER |
| Generation Screened: | X T1 Mature X T2 Seedling □T2 Mature □T3 Seedling |
| Criteria: | Bidirectionality: PASS Exons: PASS Repeats: PASS |

Table 3. Promoter utility
Utility: Translocation, seed fill. Improved loading of phloem, increased source capacity. Increased seed
yield. Cotyledon angle, improved seedling survival.
Notes: Extensive studies on plant signaling molecules over the past decade indicate that plant
cell-to-cell communication, as is the case with animal systems, makes use of small peptide signals
and specific receptors. To date, four peptide-ligand-receptor paris have been identified and shown
to be involved in a variety of processes. Matsubayashi. Ligand-receptor pairs in plant peptide
signaling. J Cell Sci. 2003 Oct 1; 116(Pt 19): 3863-70.

| | |
|---|---|
| Construct: | YP0071 |
| Promoter candidate I.D: | 11768674 |
| cDNA I.D: | 12721583 (OCKHAM3-C) |
| Lines expressing: | YP0071-01, YP0071-02 plant date 7/28/03 |

Promoter Expression Report # 101
Report Date: December 3, 2003
Promoter Tested In: Arabidopsis thaliana, WS ecotype
Spatial expression summary:

| | |
|---|---|
| Flower | H pedicel, H receptacle |
| Silique | H placentae |
| Stem | H epidermis H cortex H vascular, L pith |
| Hypocotyl | H epidermis, H vascular |
| Cotyledon | H mesophyll, H vascular, H epidermis, H hydathode |
| Rosette Leaf | H mesophyll, H vascular, H epidermis, Hprimordia |
| Primary Root | H epidermis, H cortex, H vascular |
| Lateral root | H epidermis, H cortex |

Observed expression pattern:
T1 mature: High expression in epidermis and cortical cells of stem and pedicles near
inflorescence shoot apex. Weakens near floral organs except in the placenta where GFP is
also highly expressed. Not expressed in ovules or embryos. High GFP expression in
vasculature of stem. T2 seedling: High expression throughout leaves and epidermis of
hypocotyl. No expression observed in ground tissues of hypocotyl. High epidermal,
cortex and vascular expression in root.

| | |
|---|---|
| Expected expression pattern: | Enzyme located in chloroplasts, >4 fold high in |
| | seedlings |
| Selection Criteria: | Ceres Arabidopsis microarray |
| Gene: | product = “DEF (CLA1) protein” CLA1 (for “cloroplastos alterados', or “altered |
| | chloroplasts') CLA1 encodes 1-deoxy-d-xylulose 5-phosphate synthase, |
| | which catalyses the first step of the non-mevalonate isoprenoid |
| | biosynthetic pathway. |
| GenBank: | NM_117647 Arabidopsis thaliana DEF (CLA1) protein (At4g15560) mRNA, |
| | complete cds gi\|30683316\|ref\|NM_117647.2\|[30683316] |
| Source Promoter Organism: | Arabidopsis thaliana WS |
| Vector: | pNewbin4-HAP1-GFP |
| Marker Type: | GFP-ER |
| Generation Screened: | X T1 Mature X T2 Seedling □T2 Mature □T3 Seedling |
| Criteria: | Bidirectionality: PASS Exons: PASS Repeats: PASS |

Table 3. Promoter utility
Utility: Increased photosynthetic capacity and source capacity. Larger plants. Altered plant morphology.
Altered plant metabolism. Increased seed loading and seed yield.
Notes: CLA1 encodes 1-deoxy-d-xylulose 5-phosphate synthase, which catalyses the first step of
the non-mevalonate isoprenoid biosynthetic pathway. Crowell DN, Packard CE, Pierson CA,
Giner JL, Downes BP, Chary SN. Identification of an allele of CLA1 associated with variegation
in Arabidopsis thaliana. Physiol Plant. 2003 May; 118(1): 29-37.

| | |
|---|---|
| Construct: | YP0216 |
| Promoter candidate I.D: | 13148171 |
| cDNA I.D: | 12575820 |
| Lines expressing: | YP0216-01, -02, -03, -04 plant date 05/05/03; |

Promoter Expression Report # 102
Report Date: October 30, 2003
Promoter Tested In: Arabidopsis thaliana, WS ecotype
Spatial expression summary:

| | |
|---|---|
| Ovule | Pre-fertilization: L primordia L inner integument L outer integument |
| | Post-fertilization: H suspensor |

Observed expression pattern:
T1 mature: Weak expression observed throughout ovule primordia including mother
megaspore cell. Post-fertilization expression specific to suspensor cells of embryo.
Degeneration of expression in suspensor at torpedo stage.
T2 Seedling: No expression.
Expected expression pattern: Nucellus and megaspore mother cell
Selection Criteria: Literature. Yang WC, Ye D, Xu J, Sundaresan V. The SPOROCYTELESS
gene of Arabidopsis is required for initiation of sporogenesis and encodes a novel nuclear
protein.
Genes Dev. 1999 Aug 15; 13(16): 2108-17.
Gene: Nozzle Sporocyteles
GenBank: NM_118867 Arabidopsis thaliana NOZZLE SPOROCYTELESS
(At4g27330) RNA, complete cdsgi|18416968|ref|NM_118867.1|[18416968]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling □T2 Mature □T3 Seedling
Criteria: Bidirectionality: Exons: Repeats:
Table 5. Promoter utility
Utility: Better embryo fill, larger embryo and seed. Altered seed composition. Increased seed weight and
yield. Better performing seedlings. Seedlings tolerant to stress. Altered source-sink balance.
Notes: Balasubramanian S, Schneitz K. NOZZLE links proximal-distal and adaxial-
abaxial pattern formation during ovule development in Arabidopsis thaliana.
Development. 2002 Sep; 129(18): 4291-

| | |
|---|---|
| Construct: | YP0271 |
| Promoter candidate I.D: | 11768757 |
| cDNA I.D: | 12658070 |
| Lines expressing: | YP0271-01, -02 plant date 4/14/03 |

Promoter Expression Report # 103
Report Date: October 30, 2003
Promoter Tested In: Arabidopsis thaliana, WS ecotype
Spatial expression summary:

| | |
|---|---|
| Silique | L ovule |
| Ovule | Post-fertilization: M zygote L embryo sac L embryo |
| Embryo | M suspensor L torpedo L radicle |
| Rosette Leaf | M mesophyll H epidermis H stomata |
| Primary Root | H pericycle |
| Lateral root | H initials H flanking cells H primordia |

Observed expression pattern:
T1 mature: High expression throughout mature female gametophyte at fertilization and
in embryo from zygote to torpedo stage embryo. Expression in embryo restricted to
radicle. Not observed in leaf, however this may coincide with severe yellowing of leaves
in plants screened during this time. T2 seedling: High GFP expression in mesophyll and
epidermal cells of rosette leaves. Expression in root is specific to pericycle cells and
lateral root primordia.

| | |
|---|---|
| Expected expression pattern: | Leaf |

Selection Criteria: Literature. Leaf-Specific Upregulation of Chloroplast Translocon Genes by a
CCT Motif-Containing Protein, CIA 2. Sun CW, Chen LJ, Lin LC, Li HM.Plant Cell. 2001 Sep; 13(9): 2053-2062.
PMCID: 139451 [Abstract] [Full Text] [PDF]

| | |
|---|---|
| Gene: | CIA2 |

GenBank: NM_125100 Arabidopsis thaliana CIA2 (CIA2) (At5g57180) mRNA,
complete cds
gi|30696839|ref|NM_125100.2|[30696839]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling □T2 Mature □T3 Seedling
Criteria: Bidirectionality: Exons: Repeats:
Table 5. Promoter utility
Utility: Nutrition. Imprint modulation through female, heavier seed, lighter seed, seedless fruits.
Increased lateral root growth. More lateral roots, larger lateral roots. Improved drought tolerance. Improved
performance in low-nitrogen soil, improved source capacity.
Notes:

| | |
|---|---|
| Construct: | YP0279 |
| Promoter candidate I.D: | 11768839 |
| cDNA I.D: | 12600234 (OCKHAM3-C) |
| Lines expressing: | YP00279-01, -02, -03 plant date 4/14/03 |

Promoter Expression Report # 105
Report Date: December 3, 2003
Promoter Tested In: Arabidopsis thaliana, WS ecotype
Spatial expression summary:

| | |
|---|---|
| Silique | L ovule |
| Ovule | M embryo sac |
| Leaf | L vascular |
| Hypocotyl | L vascular |
| Cotyledon | L vascular, M hydathode |
| Primary Root | L epidermis, M vascular, M pericycle |

Observed expression pattern:
T1 mature: GFP expression decreasing in female gametophyte. Low expression in leaf
vasculature. T2 seedling: Low expression in cotyledon and hypocotyl vasculature. Low
expression in root vasculature and pericycle cells.

| | |
|---|---|
| Expected expression pattern: | PEG-inducible |
| Selection Criteria: | Ceres, Inc. Arabidopsis Expression data |

Gene: Arabidopsis thaliana mitochondrial carrier protein family
GenBank: NM_118590 Arabidopsis thaliana mitochondrial carrier protein family
(At4g24570) mRNA, complete cds gi|30686585|ref|NM_118590.2|[30686585]

| | |
|---|---|
| Source Promoter Organism: | Arabidopsis thaliana WS |
| Vector: | pNewbin4-HAP1-GFP |
| Marker Type: | GFP-ER |

Generation Screened: X T1 Mature X T2 Seedling □T2 Mature □T3 Seedling
Criteria: Bidirectionality: PASS Exons: PASS Repeats: PASS
Table 3. Promoter utility
Utility: Imprint modulation through female, larger (heavier) seeds, smaller (lighter) seeds, seedless
fruits. Altered endosperm and seed composition, improved drought tolerance. Improved performance in
low-nitrogen soil.
Notes:

| | |
|---|---|
| Construct: | YP0285 |
| Promoter candidate I.D: | 11768588 |
| cDNA I.D: | 13609092 |
| Lines expressing: | YP0285-01, -02, -04 plant date 6/04/03 |

Promoter Expression Report # 106
Report Date: October 31, 2003
Promoter Tested In: Arabidopsis thaliana, WS ecotype
Spatial expression summary:

| | |
|---|---|
| Flower | H vascular |
| Silique | H vascular |
| Stem | H vascular |
| Leaf | H vascular |
| Hypocotyl | H vascular |
| Cotyledon | H vascular |
| Rosette Leaf | H vascular |
| Primary Root | H vascular, H pericycle |
| Lateral root | H pericycle H vascular |

Observed expression pattern:
T1 mature: Very high expression in vasculature of flowers, stems, and leaves. Not detected in
reproductive tissues in silique. T2 seedling: Very high expression throughout seedling
vasculature. Expression in root extending into pericycle cells.
Expected expression pattern: Shoot apical meristem
Selection Criteria: Greater than 5x down in stm microarray
Gene: Leucine-rich repeat transmembrane protein kinase
GenBank: NM_118146 Arabidopsis thaliana leucine-rich repeat transmembrane protein
kinase, putative (At4g20270) mRNA, complete cds gi|30685044|ref|NM_118146.2|[30685044]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling ? T2 Mature ? T3 Seedling
Criteria: Bidirectionality: Exons: Repeats:
Table 5. Promoter utility
Utility: Improved translocation, improved source capacity and seed fill. Heavier seeds. More seeds. Larger
siliques. Improved seed yield. Moderate nitrate and/or amino acid transport. Increased transport to floorsink.
Notes:

| | |
|---|---|
| Construct: | YP0080 |
| Promoter candidate I.D: | 11768676 |
| cDNA I.D: | 12603755 (OCKHAM3-C) |
| Lines expressing: | YP0080-01, -02, -03 plant date 7/28/03 |

Promoter Expression Report # 107
Report Date: October 31, 2003
Promoter Tested In: Arabidopsis thaliana, WS ecotype
Spatial expression summary:

| | |
|---|---|
| Rosette Leaf | L vascular M epidermis |
| Primary Root | H epidermis M root hairs |

Observed expression pattern:
T1 mature: No expression observed. Predicted expression in ovule primordium.
T2 seedling: High expression throughout root epidermal cells. Low epidermal and
vasculature expression at leaf margins.
Expected expression pattern: Integument.
Selection Criteria: Arabidopsis public: The BELL1 gene encodes a homeodomain
protein involved in pattern formation in the Arabidopsis ovule primordium.
Gene: =“homeodomain protein, BELL1 (BEL1)”
GenBank: NM_123506 Arabidopsis thaliana homeodomain protein, BELL1
(BEL1) (At5g41410) mRNA, complete cds
gi|30693794|ref|NM_123506.2|[30693794]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling ? T2 Mature ? T3
Seedling
Criteria: Bidirectionality: Exons: Repeats:
Table 5. Promoter utility
Utility: Improve ion uptake in roots.
Notes:

| | |
|---|---|
| Construct: | YP0122 |
| Promoter candidate I.D: | 11768849 |
| cDNA I.D: | 13593439 (OCKHAM3-C) |
| Lines expressing: | YP0122-01, -02 plant date 1/17/03 |

Promoter Expression Report # 116
Report Date: November 4, 2003
Promoter Tested In: Arabidopsis thaliana, WS ecotype
Spatial expression summary:

| | |
|---|---|
| Flower | L pedicel, L sepal, H carpel, H epidermis, L stomata, H silique |
| Silique | H carpel, H epidermis, L stomata, L abscission zone |
| Leaf | H mesophyll, L vascular, H epidermis |
| Primary Root | L root hairs |

Observed expression pattern:
T1 mature: GFP expression at the base of sepals at abscission zone of developing
and mature flowers. High expression specific to carpels of developing and mature
siliques. T2 seedling: Weak root hair expression at hypocotyl transition zone
observed in 1 in 6 seedlings and in only1 of 2 events screened.
Expected expression pattern: Flowers, seed, roots.
Selection Criteria: Arabidopsis public; containing AP2 DNA binding domain.
Gene: EREBP-2
GenBank: NM_124093 Arabidopsis thaliana ethylene responsive element binding
factor 2 (EREBP-2) (At5g47220) mRNA, complete cds
gi|30695135|ref|NM_124093.2|[30695135]

| | |
|---|---|
| Source Promoter Organism: | Arabidopsis thaliana WS |
| Vector: | pNewbin4-HAP1-GFP |
| Marker Type: | GFP-ER |

Generation Screened: X T1 Mature XT2 Seedling ? T2 Mature ? T3
Seedling
Criteria: Bidirectionality: PASS Exons: PASS Repeats:
Table 5. Promoter utility
Utility: Increased leaf size and volume, increased source capacity. Tolerance to drought. Improved
performance in low nitrogen conditions. Larger siliques, increased seed number. Increased seed
yield. Altered dehiscence and seed scatter.
Notes:

| | |
|---|---|
| Construct: | YP0015 |
| Promoter candidate I.D: | 11768611 |
| cDNA I.D: | 13612380 |
| Lines expressing: | YP0015-03, -04 plant date 9/8/03 |

Promoter Expression Report # 118
Report Date: November 4, 2003
Promoter Tested In: Arabidopsis thaliana, WS ecotype
Spatial expression summary:

| | |
|---|---|
| Flower | H anther H pollen L vascular H stomata |
| Silique | M ovule |
| Ovule | Pre-fertilization: H outer integument H embryo sac H gametophyte |
| | Post-fertilization: M outer integument H seed coat H embryo |
| Embryo | H suspensor H heart H late H mature L radicle L cotyledons |
| Stem | H epidermis H stomata H trichome |
| Leaf | H stomata |
| Hypocotyl | H epidermis L cortex H stomata |
| Cotyledon | H mesophyll H vascular H epidermis H stomata |
| Rosette Leaf | H stomata |
| Primary Root | H cortex |

Observed expression pattern:
T1 mature: Expressed in pollen cells throughout development. Expression visible during
pollination when dehisced pollen attaches to stigma resulting in extension of the cell wall
to establish an attachment site or “foot”. Once attached, pollen is hydrated and
germination of pollen tubes follows through the stigma at the attachment site. These
processes are likely targets in regulation of self-incompatibility and species-specific
pollen recognition. No expression is observed after hydration. Expressed in egg sac of
pre-fertilized ovules, inner integument, endosperm, heart stage embryo and suspensor
cells of developing ovules. High specific expression in and epidermal cell files flanking
trichomes of stem and guard cells throughout mature plant. T2 seedling: High expression
in epidermal and guard cells throughout seedling. High expression epidermal, vascular,
and mesophyll cells of cotyledons. Not observed in leaf primordia. High expression
specific to cortical cells of root.
Expected expression pattern: Induced prior to cell division and usually associated with
dividing cells.
Selection Criteria: Ceres BLAST search homology; CDC2-like protein
Gene: putative protein kinase/note = “similar to cyclin-dependent kinase cdc2MsE
[Medicago sativa]
GenBank: NM_125756 Arabidopsis thaliana protein kinase, putative (At5g63610)
mRNA, complete cds gi|30697871|ref|NM_125756.2|[30697871]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling □T2 Mature □T3 Seedling
Criteria: Bidirectionality: Exons: Repeats:

| | |
|---|---|
| Table 5. | Promoter utility |

Utility: Imprint modulation through male, larger (heavier) seeds, smaller (lighter) seeds. Male sterility
and altered breeding barriers. Altered pollen composition. Altered fertility. Resistance to drought.
Notes: Abstracts; Elucidating the complete molecular determination of a self-incompatibility (SI)
system in plants has grown significantly with recent investigations of the sporophytic
incompatibility system in the genus Brassica. Male (pollen) and female (stigma) components of
the recognition/incompatibility reaction appear to be controlled by separate genes that reside in a
small genomic region (the S-locus; see YU et al. 1996 [symbol] ; SCHOPFER et al. 1999 [symbol] ). The
interaction between male and female components is not completely understood, but it is thought
that a pollen surface protein acts as a ligand that is recognized by a transmembrane protein in the
papillary cells on the surface of the stigma. When the pollen and pistil specificities are from the
same S-allele, pollen tube growth is inhibited. The stigma component of this recognition system is
now thought to be the S-locus receptor kinase, encoded by the SRK gene. This protein has an
extracellular glycoprotein domain and an intracellular serine-threonine protein kinase (STEIN et
al. 1991 [symbol] ) and has been shown to be necessary, and perhaps sufficient, for determining
specificity (CUI et al. 2000 [symbol] ; TAKASAKI et al. 2000 [symbol] ). A second protein, S-locus glycoprotein,
encoded by the closely linked SLG gene, is not in itself sufficient for determining specificity,
although it may be necessary for proper rejection of incompatible pollen (SHIBA et al. 2000 [symbol] ;
TAKASAKI et al. 2000 [symbol] ). SLG sequences show homology to those of the first exon of SRK (the
S-domain). A pollen coat protein, encoded by the linked SCR gene, has recently been shown to be
necessary and sufficient for determination of the pollen specificity (SCHOPFER et al. 1999 [symbol] ;
TAKAYAMA et al. 2000 [symbol] ). Genetics, Vol. 158, 387-399, May 2001, Copyright © 2001
SCHIERUP, M. H., B. K. MABLE, P. AWADALLA, and D. CHARLESWORTH, 2001
Identification and characterization of a polymorphic receptor kinase gene linked to the self-
incompatibility locus of Arabidopsis lyrata.. Genetics 158: 387-399. [Abstract/Free Full Text]
SCHOPFER, C. R., M. E. NASRALLAH, and J. B. NASRALLAH, 1999 The male determinant
of self-incompatibility in Brassica. Science 286: 1697-1700[Abstract/Free Full Text]. STEIN, J.,
B. HOWLETT,
D. C. BOYES, M. E. NASRALLAH, and J. B. NASRALLAH, 1991 Molecular
cloning of a putative receptor protein kinase gene encoded at the self-incompatibility locus of
Brassica oleracea. Proc. Natl. Acad. Sci. USA 88: 8816-8820[Abstract].

| | |
|---|---|
| Construct: | YP0230 |
| Promoter candidate I.D: | 13148201 |
| cDNA I.D: | 12676237 |
| Lines expressing: | YP0230-02, -03 (9/08/03) |

Promoter Expression Report # 119
Report Date: December 3, 2003
Promoter Tested In: Arabidopsis thaliana, WS ecotype
Spatial expression summary:

| | |
|---|---|
| Silique | H ovule |
| Ovule | Pre-fertilization: H outer integument |
| | Post-fertilization: H outer integument, H seed coat |

Observed expression pattern:
T1 Mature: GFP expressed in outer integument early in ovule development through seed
coat of mature seeds. T2 Seedling: No expression observed.

| | |
|---|---|
| Expected expression pattern: | Expressed in ovules and different parts of seeds |
| Selection Criteria: | Greater than 50x up in pi ovule microarray |
| Gene: | “hypothetical protein/product = “expressed protein” |

GenBank: NM_117365 Arabidopsis thaliana expressed protein (At4g12960) mRNA,
complete cds gi|30682287|ref|NM_117365.2|[30682287]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling □T2 Mature □T3 Seedling
Criteria: Bidirectionality: PASS Exons: PASS Repeats: PASS

| | |
|---|---|
| Table 3. | Promoter utility |

Utility: Seed size determination. Increased seed size. Altered seed composition. Tolerance of seeds to
desiccation. Resistance of seeds to abortion. Increase sink strength by expression of A.A. transporters

| | |
|---|---|
| Construct: | YP0120 |
| Promoter candidate I.D: | 11768656 |
| cDNA I.D: | 12370095 |
| Lines expressing: | YP0120-01, -02 Plant date 9/8/03 |

Promoter Expression Report # 120
Report Date: November 4, 2003
Promoter Tested In: Arabidopsis thaliana, WS ecotype
Spatial expression summary:

| | |
|---|---|
| Flower | H sepal |
| Rosette Leaf | H epidermis H stomata |

Observed expression pattern:
T1 Mature: High epidermal expression in petals of developing and mature flowers.
Not detected in other organs. T2 Seedlings: High expression in epidermal cells of
initial leaf primordia.
Expected expression pattern: Emerging true leaves.
Selection Criteria: Literature; Cho HT, Cosgrove DJ Altered expression of expansin
modulates leaf growth and pedicel abscission in Arabidopsis thaliana. Proc Natl Acad Sci USA.
2000 Aug 15; 97(17): 9783-8.
Gene: product = “expansin, putative (EXP 10)”
GenBank: NM_102440 Arabidopsis thaliana expansin, putative (EXP 10)
(At1g26770) mRNA, complete cds gi|30689629|ref|NM_102440.2|[30689629]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling ? T2 Mature ? T3 Seedling
Criteria: Bidirectionality: Exons: Repeats:

| | |
|---|---|
| Table 5. | Promoter utility |

Utility: Leaf size and photosynthetic capacity. Increased source strength. Increased sucrose
loading. Increased leaf expansion, resulting in improved seedling stress tolerance. Modulate size of
organs, young leaf specific exp.
Notes:

| | |
|---|---|
| Construct: | YP0261 |
| Promoter candidate I.D: | 11768750 |
| cDNA I.D: | 12385291 |
| Lines expressing: | YP0261-01, -03 plant date 09/08/03 |

Promoter Expression Report # 121
Report Date: November 5, 2003
Promoter Tested In: Arabidopsis thaliana, WS ecotype
Spatial expression summary:

| | |
|---|---|
| Flower | H pedicel H receptacle H filament Hanther H carpel H vascular H silique |
| Silique | H vascular |
| Stem | H vascular |
| Leaf | H vascular |
| Hypocotyl | L epidermis H vascular |
| Cotyledon | H vascular L epidermis |
| Rosette Leaf | H epidermis |
| Primary Root | H cortex H root cap |

Observed expression pattern:
T1 Mature: High GFP expression in vasculature of stem and leaves, also pedicles,
siliques and stamen of flowers. Not detected in sepals and petals. Expression in silique
specific to medial vasculature. T2 Seedling: High GFP expression in hypocotyl and
cotyledon vasculature and cortex of root. Not observed in root vasculature.
Expected expression pattern: Stem and root elongation zones.
Selection Criteria: Hanazawa Y, Takahashi T, Michael AJ, Burtin D, Long D, Pineiro M, Coupland G,
Komeda Y. ACAULIS5, an Arabidopsis gene required for stem elongation, encodes a
spermine synthase. EMBO J. 2000 Aug 15; 19(16): 4248-56.
Gene: Arabidopsis ACAULIS5 (ACL5)
GenBank: NM_121958 Arabidopsis thaliana spermine synthase (ACL5) (At5g19530)
mRNA, complete cds gi|30687363|ref|NM_121958.2|[30687363]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling □T2 Mature □T3 Seedling
Criteria: Bidirectionality: Exons: Repeats:

| | |
|---|---|
| Table 5. | Promoter utility |

Utility: Translocation to the flower, seed fill. Sucrose loading and transport. Improved source capacity,
lending to larger plant organs, larger plants, Increased biomass, increased yield. Improved root growth and
soil penetration. Resistance to drought, improved uptake of nitrogen and phosphate. Modify nitrate uptake
and translocation to Xylem.

| | |
|---|---|
| Construct: | YP0263 |
| Promoter candidate I.D: | 11768752 |
| cDNA I.D: | 12640578 |
| Lines expressing: | YP0263-10, -11, -12 plant date 9/8/03 |

Promoter Expression Report # 123
Report Date: November 5, 2003
Promoter Tested In: Arabidopsis thaliana, WS ecotype
Spatial expression summary:

| | |
|---|---|
| Flower | M stomata |
| Silique | M stomata |
| Ovule | Post-fertilization: L embryo |
| Embryo | L provascular, L cotyledons |
| Primary Root | L epidermis, L xylem |

Observed expression pattern:
T1 mature: Strong expression in embryonic vascular tissue of cotyledons.
Weak guard cell expression in flower pedicle and silique. T2 seedling: Weak
degrading root epidermis expression near transition zone. Weak root vascular
expression in elongation zone. Expression in very thin cell layer appears to be
xylem.
Expected expression pattern: root, flowers, ovules, young silique
Selection Criteria: Arabidopsis Two component line CS9135 (see notes).
Gene: Hypothetical protein containing helix-loop-helix DNA binding domain.
GenBank: NM_116493 Arabidopsis thaliana bHLH protein (At4g02590) mRNA,
complete cds
gi|30679204|ref|NM_116493.2|[30679204]

| | |
|---|---|
| Source Promoter Organism: | Arabidopsis thaliana WS |
| Vector: | pNewbin4-HAP1-GFP |
| Marker Type: | GFP-ER |

Generation Screened: XT1 Mature XT2 Seedling ? T2 Mature ? T3 Seedling
Criteria: Bidirectionality: Exons: Repeats:

| | |
|---|---|
| Table 5. | Promoter utility |

Utility: Translocation to cotyledons, seed fill. Increased embryo and seed weight. Altered embryo
and seed composition. Improved seedling vigor, seedling resistance to drought, cold, cold/wet
conditions.

| | |
|---|---|
| Construct: | YP0003 |
| Promoter candidate I.D: | 13148213 |
| cDNA I.D: | 12649228 |
| Lines expressing: | YP0003-04, -06 plant date 9/8/03 |

TABLE 3

| cDNA ID | Expt_Rep_ID | Short_Name | Differential |
|---|---|---|---|
| 13610584 | 20000264 | At_Open_Flower | − |
| 12656458 | 20000708 | At_Fis1_Siliques | − |
| 4909806 | 20000439 | At_Roots | − |
| 4909806 | 20000185 | At_Roots_YF | − |
| 12669615 | 20000264 | At_Open_Flower | − |
| 4909806 | 20000794 | At_Petals | − |
| 12711515 | 20000794 | At_Petals | − |
| 12669615 | 20000265 | At_Open_Flower | − |
| 4909291 | 20000092 | At_42deg_Heat | − |
| 13612879 | 20000185 | At_Roots | − |
| 13489977 | 20000234 | At_Siliques | − |
| 13610584 | 20000794 | At_Petals | − |
| 13612879 | 20000438 | At_Shoots | − |
| 12669615 | 20000234 | At_Siliques | − |
| 13489977 | 20000264 | At_Open_Flower | − |
| 12669615 | 20000286 | At_Open_Flower | − |
| 12329827 | 20000439 | At_Roots | − |
| 13610584 | 20000286 | At_Open_Flower | − |
| 13610584 | 20000234 | At_Siliques | − |
| 13612879 | 20000439 | At_Roots | − |
| 12688453 | 20000439 | At_Roots | − |
| 12669615 | 20000794 | At_Petals | − |
| 12688453 | 20000185 | At_Roots | − |
| 12329827 | 20000185 | At_Roots | − |
| 12692181 | 20000314 | At_14day_Shoots-Roots | − |
| 13489977 | 20000286 | At_Open_Flower | − |
| 4909291 | 20000457 | At_42deg_Heat | − |
| 12370148 | 20000234 | At_Siliques | − |
| 13489977 | 20000265 | At_Open_Flower | − |
| 13609817 | 108434 | At_Root_Tips | − |
| 12348737 | 20000794 | At_Petals | − |
| 12713856 | 20000439 | At_Roots | − |
| 12333534 | 20000794 | At_Petals | − |
| 13612879 | 20000184 | At_Shoots | − |
| 13489977 | 20000438 | At_Shoots | − |
| 12692181 | 108457 | At_Diversity_Expt | − |
| 13489977 | 20000235 | At_Siliques | − |
| 12669615 | 20000235 | At_Siliques | − |
| 13489977 | 20000326 | At_Pollen | − |
| 13489977 | 20000236 | At_Siliques | − |
| 12713856 | 20000185 | At_Roots | − |
| 13612879 | 20000527 | At_10%_PEG | − |
| 13612879 | 20000794 | At_Petals | − |
| 4909806 | 108435 | At_stm_Mutants | − |
| 13610584 | 20000235 | At_Siliques | − |
| 12370148 | 20000235 | At_Siliques | − |
| 12669615 | 20000438 | At_Shoots | − |
| 12322657 | 20000438 | At_Shoots | − |
| 12692181 | 20000315 | At_14day_Shoots-Roots | − |
| 12370148 | 20000265 | At_Open_Flower | − |
| 12679922 | 20000439 | At_Roots | − |
| 12678173 | 20001654 | At_Interploidy_Crosses | − |
| 13612919 | 108595 | At_Ler-pi_Ovule | − |
| 12370148 | 20000286 | At_Open_Flower | − |
| 12670159 | 20000185 | At_Roots | − |
| 12333534 | 20001654 | At_Interploidy_Crosses | − |
| 12713856 | 20000071 | At_100uM_ABA_Mutants | − |
| 12711515 | 20000214 | At_4deg_Cold | − |
| 12322657 | 20000326 | At_Pollen_YF_07-12-02_P | − |
| 12679922 | 20000234 | At_Siliques_YF_6-05-02_P | − |
| 12679922 | 20000185 | At_Roots_YF_7-24-02_P | − |
| 12370148 | 20000236 | At_Siliques_YF_6-05-02_P | − |
| 12660077 | 20001248 | At_Far-red-induction_AM_4-16-03_P | − |
| 12713856 | 108595 | At_Ler-pi_Ovule_RP_8-24-00_cDNA_P | − |
| 13612919 | 20000326 | At_Pollen_YF_07-12-02_P | − |
| 12679922 | 20000265 | At_Open_Flower_YF_06-19-02_P | − |
| 12348737 | 20000264 | At_Open_Flower_YF_06-19-02_P | − |
| 12669615 | 20000236 | At_Siliques_YF_6-05-02_P | − |
| 12679922 | 20001556 | At_Drought_Soil_Dry_YF_07-16-03_P | − |
| 12711515 | 20001554 | At_Drought_Soil_Dry_YF_07-16-03_P | − |
| 12711515 | 20000264 | At_Open_Flower_YF_06-19-02_P | − |
| 12678173 | 20000223 | At_CS6632_Shoots-Roots_SK_5-29-02_cDNA_P | − |
| 12713856 | 20000117 | At_100uM_ABA_Mutants_YF_2-4-02_cDNA_P | − |
| 13612879 | 20000458 | At_42deg_Heat_YF_7-29-02_P | − |
| 12329827 | 20000245 | At_Caf_Knockout_RS_6-13-02_P | − |
| 13612919 | 20000439 | At_Roots_YF_7-24-02_P | − |
| 12322657 | 20000184 | At_Shoots_YF_7-24-02_P | − |
| 12646933 | 20000185 | At_Roots_YF_7-24-02_P | − |
| 12646933 | 20000438 | At_Shoots_YF_7-24-02_P | − |
| 12679922 | 20000286 | At_Open_Flower_YF_06-19-02_P | − |
| 12576899 | 20000458 | At_42deg_Heat_YF_7-29-02_P | − |
| 12348737 | 20000235 | At_Siliques_YF_6-05-02_P | − |
| 12348737 | 108595 | At_Ler-pi_Ovule_RP_8-24-00_cDNA_P | − |
| 13609817 | 108457 | At_Diversity_Expt_RP_5-16-01_cDNA_P | − |
| 12660077 | 20000185 | At_Roots_YF_7-24-02_P | − |
| 12664333 | 20000527 | At_10%_PEG_YF_7-29-02_P | − |
| 13613553 | 20000180 | At_Germinating_Seeds_YF_4-11-02_P | − |
| 13647840 | 20000438 | At_Shoots_YF_7-24-02_P | − |
| 12679922 | 20001555 | At_Drought_Soil_Dry_YF_07-16-03_P | − |
| 12370148 | 20000439 | At_Roots_YF_7-24-02_P | − |
| 12348737 | 20000708 | At_Fis1_Siliques_RP_01-08-03_P | − |
| 12711515 | 20001556 | At_Drought_Soil_Dry_YF_07-16-03_P | − |
| 12333534 | 108577 | At_42deg_Heat_YF_8-24-00_cDNA_P | − |
| 12329827 | 20000438 | At_Shoots_YF_7-24-02_P | − |
| 12348737 | 108435 | At_stm_Mutants_RP_5-2-01_cDNA_P | − |
| 12679922 | 20000236 | At_Siliques_YF_6-05-02_P | − |
| 12713856 | 20001248 | At_Far-red-induction_AM_4-16-03_P | − |
| 4909806 | 20001557 | At_Drought_Soil_Dry_YF_07-16-03_P | − |
| 12711515 | 20001555 | At_Drought_Soil_Dry_YF_07-16-03_P | − |
| 13619323 | 20000438 | At_Shoots_YF_7-24-02_P | − |
| 12679922 | 20000264 | At_Open_Flower_YF_06-19-02_P | − |
| 13613553 | 20001247 | At_Far-red-induction_AM_4-16-03_P | − |
| 12455436 | 108462 | At_Germinating_Seeds_YF_5-22-01_cDNA_P | − |
| 13612919 | 20001450 | At_Far-red-induction_AM_4-16-03_P | − |
| 12332135 | 20000439 | At_Roots_YF_7-24-02_P | − |
| 12332135 | 20000438 | At_Shoots_YF_7-24-02_P | − |
| 13647840 | 20000185 | At_Roots_YF_7-24-02_P | − |
| 12576899 | 20001555 | At_Drought_Soil_Dry_YF_07-16-03_P | − |
| 12455436 | 108464 | At_Germinating_Seeds_YF_5-22-01_cDNA_P | − |
| 13612919 | 20001248 | At_Far-red-induction_AM_4-16-03_P | − |
| 13647840 | 20000708 | At_Fis1_Siliques_RP_01-08-03_P | − |
| 12370148 | 20000264 | At_Open_Flower_YF_06-19-02_P | − |
| 13609817 | 20001558 | At_Drought_Soil_Dry_YF_07-16-03_P | − |
| 12329827 | 20000794 | At_Petals_YF_03-06-03_P | − |
| 13489977 | 20000184 | At_Shoots_YF_7-24-02_P | − |
| 13489977 | 20000180 | At_Germinating_Seeds_YF_4-11-02_P | − |
| 13613553 | 108464 | At_Germinating_Seeds_YF_5-22-01_cDNA_P | − |
| 4909806 | 20001560 | At_Drought_Soil_Dry_YF_07-16-03_P | − |
| 12678173 | 108457 | At_Diversity_Expt_RP_5-16-01_cDNA_P | − |
| 13613553 | 20000438 | At_Shoots_YF_7-24-02_P | − |
| 12370148 | 20000185 | At_Roots_YF_7-24-02_P | − |
| 13613553 | 20000185 | At_Roots_YF_7-24-02_P | − |
| 4909806 | 20001558 | At_Drought_Soil_Dry_YF_07-16-03_P | − |
| 12576899 | 20000173 | At_42deg_Heat_YF_4-11-02_P | − |
| 12370148 | 20000438 | At_Shoots_YF_7-24-02_P | − |
| 12332135 | 20000265 | At_Open_Flower_YF_06-19-02_P | − |
| 12333534 | 20000185 | At_Roots_YF_7-24-02_P | − |
| 13489977 | 20000439 | At_Roots_YF_7-24-02_P | − |
| 13617784 | 20000234 | At_Siliques_YF_6-05-02_P | − |
| 4909806 | 108577 | At_42deg_Heat_YF_8-24-00_cDNA_P | − |
| 4909806 | 108595 | At_Ler-pi_Ovule_RP_8-24-00_cDNA_P | − |
| 12455436 | 108463 | At_Germinating_Seeds_YF_5-22-01_cDNA_P | − |
| 12703041 | 20001559 | At_Drought_Soil_Dry_YF_07-16-03_P | − |
| 12713856 | 20000086 | At_100uM_ABA_Mutants_YF_2-4-02_cDNA_P | − |
| 12711515 | 20000213 | At_4deg_Cold_AS_5-17-02_P | − |
| 12703041 | 20000234 | At_Siliques_YF_6-05-02_P | − |
| 13489977 | 20000185 | At_Roots_YF_7-24-02_P | − |
| 12669615 | 20000527 | At_10%_PEG_YF_7-29-02_P | − |
| 12679922 | 108501 | At_ap2_floral_buds_DJ_7-10-01_cDNA_P | − |
| 13612919 | 108435 | At_stm_Mutants_RP_5-2-01_cDNA_P | − |
| 12333534 | 108579 | At_4deg_Cold_YF_8-24-00_cDNA_P | − |
| 12670159 | 20000265 | At_Open_Flower_YF_06-19-02_P | − |
| 12396394 | 20001557 | At_Drought_Soil_Dry_YF_07-16-03_P | − |
| 12646933 | 20000439 | At_Roots_YF_7-24-02_P | − |
| 12735519 | 20001248 | At_Far-red-induction_AM_4-16-03_P | − |
| 13609817 | 20001557 | At_Drought_Soil_Dry_YF_07-16-03_P | − |
| 12348737 | 20000236 | At_Siliques_YF_6-05-02_P | − |
| 12713856 | 20000069 | At_100uM_ABA_Mutants_YF_2-4-02_cDNA_P | − |
| 12713856 | 108573 | At_Drought_YF_8-24-00_cDNA_P | − |
| 12370148 | 20000184 | At_Shoots_YF_7-24-02_P | − |
| 13619323 | 20000184 | At_Shoots_YF_7-24-02_P | − |
| 13610584 | 20000236 | At_Siliques_YF_6-05-02_P | − |
| 13610584 | 20000437 | At_Drought_YF_06-25-02_P | − |
| 12713856 | 20000088 | At_100uM_ABA_Mutants_YF_2-4-02_cDNA_P | − |
| 13609817 | 20000265 | At_Open_Flower_YF_06-19-02_P | − |
| 12711515 | 20000236 | At_Siliques_YF_6-05-02_P | − |
| 12333534 | 108595 | At_Ler-pi_Ovule_RP_8-24-00_cDNA_P | − |
| 13609817 | 20000227 | At_Root-Tips-vs-Tops_SK_5-30-02_P | − |
| 12713856 | 20000326 | At_Pollen_YF_07-12-02_P | − |
| 12679922 | 20001559 | At_Drought_Soil_Dry_YF_07-16-03_P | − |
| 12679922 | 20000214 | At_4deg_Cold_AS_5-17-02_P | − |
| 13647840 | 20000245 | At_Caf_Knockout_RS_6-13-02_P | − |
| 12703041 | 20001556 | At_Drought_Soil_Dry_YF_07-16-03_P | − |
| 12703041 | 108595 | At_Ler-pi_Ovule_RP_8-24-00_cDNA_P | − |
| 12370148 | 20000573 | At_100uM_ABA_Mutants_YF_9-9-02_P | − |
| 13612879 | 20000326 | At_Pollen_YF_07-12-02_P | − |
| 12348737 | 20000234 | At_Siliques_YF_6-05-02_P | − |
| 4909806 | 108585 | At_5mM_NaNP_YF_8-24-00_cDNA_P | − |
| 12348737 | 20000265 | At_Open_Flower_YF_06-19-02_P | − |
| 13614559 | 20000180 | At_Germinating_Seeds_YF_4-11-02_P | − |
| 13612879 | 108668 | At_2mM_SA_YF_11-28-01_cDNA_P | − |
| 13489977 | 20000573 | At_100uM_ABA_Mutants_YF_9-9-02_P | − |
| 13613553 | 20000184 | At_Shoots_YF_7-24-02_P | − |
| 12713856 | 20000070 | At_100uM_ABA_Mutants_YF_2-4-02_cDNA_P | − |
| 12670159 | 20000264 | At_Open_Flower_YF_06-19-02_P | − |
| 13491988 | 20000227 | At_Root-Tips-vs-Tops_SK_5-30-02_P | − |
| 13609817 | 20000234 | At_Siliques_YF_6-05-02_P | − |
| 12329827 | 108462 | At_Germinating_Seeds_YF_5-22-01_cDNA_P | − |
| 12703041 | 108434 | At_Root_Tips_RP_5-1-01_cDNA_P | − |
| 13617784 | 20000286 | At_Open_Flower_YF_06-19-02_P | − |
| 12703041 | 20000437 | At_Drought_YF_06-25-02_P | − |
| 13653114 | 20000307 | At_Germinating_Seeds_YF_07-03-02_Sub_P | − |
| 12670159 | 20000439 | At_Roots_YF_7-24-02_P | − |
| 12656458 | 20001653 | At_Interploidy_Crosses_RP_04-28-03_P | − |
| 12711515 | 20000437 | At_Drought_YF_06-25-02_P | − |
| 13489977 | 20000179 | At_Germinating_Seeds_YF_4-11-02_P | − |
| 12396394 | 108595 | At_Ler-pi_Ovule_RP_8-24-00_cDNA_P | − |
| 13491988 | 20000185 | At_Roots_YF_7-24-02_P | − |
| 12711515 | 108668 | At_2mM_SA_YF_11-28-01_cDNA_P | − |
| 13610584 | 20000495 | At_Guard_Cells_JD_8-13-02_P | − |
| 13617784 | 20000236 | At_Siliques_YF_6-05-02_P | − |
| 12348737 | 20000286 | At_Open_Flower_YF_06-19-02_P | − |
| 12688453 | 20000326 | At_Pollen_YF_07-12-02_P | − |
| 13617784 | 20000264 | At_Open_Flower_YF_06-19-02_P | − |
| 12370148 | 20000173 | At_42deg_Heat_YF_4-11-02_P | − |
| 13617784 | 20000265 | At_Open_Flower_YF_06-19-02_P | − |
| 12370148 | 20000574 | At_100uM_ABA_Mutants_YF_9-9-02_P | − |
| 12333534 | 20000111 | At_42deg_Heat_YF_2-20-02_cDNA_P | − |
| 13610584 | 20001557 | At_Drought_Soil_Dry_YF_07-16-03_P | − |
| 4909291 | 108488 | At_50mM_NH4NO3_L-to-H_Rosette_EK_6-18-01_cDNA_P | − |
| 12333534 | 20000227 | At_Root-Tips-vs-Tops_SK_5-30-02_P | − |
| 12396394 | 20001248 | At_Far-red-induction_AM_4-16-03_P | − |
| 12713856 | 20000087 | At_100uM_ABA_Mutants_YF_2-4-02_cDNA_P | − |
| 12332135 | 20000185 | At_Roots_YF_7-24-02_P | − |
| 13612919 | 20000185 | At_Roots_YF_7-24-02_P | − |
| 12576899 | 20000214 | At_4deg_Cold_AS_5-17-02_P | − |
| 13617784 | 20000235 | At_Siliques_YF_6-05-02_P | − |
| 13489977 | 20000708 | At_Fis1_Siliques_RP_01-08-03_P | − |
| 12660077 | 20000573 | At_100uM_ABA_Mutants_YF_9-9-02_P | − |
| 12322657 | 20000185 | At_Roots_YF_7-24-02_P | − |
| 12370148 | 20000171 | At_42deg_Heat_YF_4-11-02_P | − |
| 12669615 | 20000458 | At_42deg_Heat_YF_7-29-02_P | − |
| 13619323 | 108474 | At_Drought_Flowers_YF_5-23-01_cDNA_P | − |
| 12333534 | 20000173 | At_42deg_Heat_YF_4-11-02_P | − |
| 4909806 | 20001459 | At_50mM_NH4NO3_L-to-H_RS-GM_05-19-03_P | − |
| 13647840 | 20000439 | At_Roots_YF_7-24-02_P | − |
| 12329827 | 108463 | At_Germinating_Seeds_YF_5-22-01_cDNA_P | − |
| 12679922 | 108577 | At_42deg_Heat_YF_8-24-00_cDNA_P | − |
| 12679922 | 20000268 | At_100mM_NaCl_YF_6-27-02_P | − |
| 12370148 | 20000180 | At_Germinating_Seeds_YF_4-11-02_P | − |
| 12370148 | 108434 | At_Root_Tips_RP_5-1-01_cDNA_P | − |
| 12322657 | 20001247 | At_Far-red-induction_AM_4-16-03_P | − |
| 13610584 | 20000438 | At_Shoots_YF_7-24-02_P | − |
| 13647840 | 20000184 | At_Shoots_YF_7-24-02_P | − |
| 13613553 | 20001451 | At_Far-red-induction_AM_4-16-03_P | − |
| 4905097 | 20001654 | At_Interploidy_Crosses_RP_04-28-03_P | − |
| 12703041 | 20000265 | At_Open_Flower_YF_06-19-02_P | − |
| 12396394 | 108579 | At_4deg_Cold_YF_8-24-00_cDNA_P | − |
| 12713856 | 20000794 | At_Petals_YF_03-06-03_P | − |
| 12646933 | 20000245 | At_Caf_Knockout_RS_6-13-02_P | − |
| 12370148 | 20000268 | At_100mM_NaCl_YF_6-27-02_P | − |
| 12333534 | 20000437 | At_Drought_YF_06-25-02_P | − |
| 12713856 | 108499 | At_DMT-II_YF_7-6-01_cDNA_P | − |
| 12678173 | 20000030 | At_CS6630_Roots_MC_1-17-02_cDNA_P | − |
| 12669615 | 20000180 | At_Germinating_Seeds_YF_4-11-02_P | − |
| 4909806 | 108668 | At_2mM_SA_YF_11-28-01_cDNA_P | − |
| 12703041 | 20001555 | At_Drought_Soil_Dry_YF_07-16-03_P | − |
| 13491988 | 20000439 | At_Roots_YF_7-24-02_P | − |
| 13610584 | 20000451 | At_CS6879_Shoots-Roots_SK_7-29-02_P | − |
| 12711515 | 108499 | At_DMT-II_YF_7-6-01_cDNA_P | − |
| 13612879 | 20001556 | At_Drought_Soil_Dry_YF_07-16-03_P | − |
| 12332135 | 20000184 | At_Shoots_YF_7-24-02_P | − |
| 12670159 | 20000794 | At_Petals_YF_03-06-03_P | − |
| 12646933 | 20001654 | At_Interploidy_Crosses_RP_04-28-03_P | − |
| 12348737 | 108579 | At_4deg_Cold_YF_8-24-00_cDNA_P | − |
| 12370148 | 20000111 | At_42deg_Heat_YF_2-20-02_cDNA_P | − |
| 12576899 | 20001556 | At_Drought_Soil_Dry_YF_07-16-03_P | − |
| 12329827 | 20000184 | At_Shoots_YF_7-24-02_P | − |
| 12333534 | 20000439 | At_Roots_YF_7-24-02_P | − |
| 12370148 | 20000708 | At_Fis1_Siliques_RP_01-08-03_P | − |
| 13613553 | 20000439 | At_Roots_YF_7-24-02_P | − |
| 12322657 | 20000439 | At_Roots_YF_7-24-02_P | − |
| 13610584 | 108434 | At_Root_Tips_RP_5-1-01_cDNA_P | − |
| 12713856 | 108584 | At_5mM_NaNP_YF_8-24-00_cDNA_P | − |
| 12333534 | 108499 | At_DMT-II_YF_7-6-01_cDNA_P | − |
| 13609817 | 20001560 | At_Drought_Soil_Dry_YF_07-16-03_P | − |
| 12670159 | 20000286 | At_Open_Flower_YF_06-19-02_P | − |
| 4909291 | 20000286 | At_Open_Flower_YF_06-19-02_P | − |
| 12396394 | 20000070 | At_100uM_ABA_Mutants_YF_2-4-02_cDNA_P | − |
| 12679922 | 20000180 | At_Germinating_Seeds_YF_4-11-02_P | − |
| 12711515 | 108435 | At_stm_Mutants_RP_5-2-01_cDNA_P | − |
| 4909806 | 108576 | At_42deg_Heat_YF_8-24-00_cDNA_P | − |
| 12332135 | 20000527 | At_10%_PEG_YF_7-29-02_P | − |
| 4909806 | 20000264 | At_Open_Flower_YF_06-19-02_P | − |
| 12711515 | 20000235 | At_Siliques_YF_6-05-02_P | − |
| 12711515 | 108461 | At_Germinating_Seeds_YF_5-22-01_cDNA_P | − |
| 13489977 | 20000574 | At_100uM_ABA_Mutants_YF_9-9-02_P | − |
| 13614841 | 20000437 | At_Drought_YF_06-25-02_P | − |
| 12711515 | 20000438 | At_Shoots_YF_7-24-02_P | − |
| 12703041 | 20000214 | At_4deg_Cold_AS_5-17-02_P | − |
| 12646933 | 20000184 | At_Shoots_YF_7-24-02_P | − |
| 13610584 | 20001300 | At_Line_Comparisons_NA_03-31-03_P | − |
| 12333534 | 20000117 | At_100uM_ABA_Mutants_YF_2-4-02_cDNA_P | − |
| 12656458 | 20001316 | At_Interploidy_Crosses_RP_04-28-03_P | − |
| 12679922 | 20000308 | At_100mM_NaCl_YF_6-27-02_P | − |
| 12678173 | 108435 | At_stm_Mutants_RP_5-2-01_cDNA_P | − |
| 12669615 | 20000244 | At_Caf_Knockout_RS_6-13-02_P | − |
| 12333534 | 20000069 | At_100uM_ABA_Mutants_YF_2-4-02_cDNA_P | − |
| 12329827 | 20000265 | At_Open_Flower_YF_06-19-02_P | − |
| 13613553 | 108463 | At_Germinating_Seeds_YF_5-22-01_cDNA_P | − |
| 12703041 | 108594 | At_Ler-rhl_Root_RP_8-24-00_cDNA_P | − |
| 13617784 | 20000326 | At_Pollen_YF_07-12-02_P | − |
| 12333534 | 20001556 | At_Drought_Soil_Dry_YF_07-16-03_P | − |
| 12396394 | 20001558 | At_Drought_Soil_Dry_YF_07-16-03_P | − |
| 12329827 | 108461 | At_Germinating_Seeds_YF_5-22-01_cDNA_P | − |
| 12332135 | 20001654 | At_Interploidy_Crosses_RP_04-28-03_P | − |
| 12711515 | 20000173 | At_42deg_Heat_YF_4-11-02_P | − |
| 12679922 | 20001560 | At_Drought_Soil_Dry_YF_07-16-03_P | − |
| 12679922 | 20000438 | At_Shoots_YF_7-24-02_P | − |
| 13613553 | 108488 | At_50mM_NH4NO3_L-to-H_Rosette_EK_6-18-01_cDNA_P | − |
| 12325134 | 108474 | At_Drought_Flowers_YF_5-23-01_cDNA_P | − |
| 13612919 | 20000070 | At_100uM_ABA_Mutants_YF_2-4-02_cDNA_P | − |
| 12333534 | 108584 | At_5mM_NaNP_YF_8-24-00_cDNA_P | − |
| 13612919 | 20000794 | At_Petals_YF_03-06-03_P | − |
| 12679922 | 20001300 | At_Line_Comparisons_NA_03-31-03_P | − |
| 13610584 | 108478 | At_Shoot_Apices_YF_6-5-01_cDNA_P | − |
| 12370148 | 108606 | At_100uM_ABA_YF_9-18-01_cDNA_P | − |
| 13612919 | 20000088 | At_100uM_ABA_Mutants_YF_2-4-02_cDNA_P | − |
| 13613553 | 108435 | At_stm_Mutants_RP_5-2-01_cDNA_P | − |
| 12396394 | 20000088 | At_100uM_ABA_Mutants_YF_2-4-02_cDNA_P | − |
| 12703041 | 108499 | At_DMT-II_YF_7-6-01_cDNA_P | − |
| 12713856 | 108572 | At_Drought_YF_8-24-00_cDNA_P | − |
| 12333534 | 20000436 | At_Drought_YF_06-25-02_P | − |
| 12711515 | 20000456 | At_100uM_BA_YF_7-29-02_P | − |
| 13613553 | 108434 | At_Root_Tips_RP_5-1-01_cDNA_P | − |
| 12561142 | 20000794 | At_Petals_YF_03-06-03_P | − |
| 13613553 | 108462 | At_Germinating_Seeds_YF_5-22-01_cDNA_P | − |
| 12576899 | 20000213 | At_4deg_Cold_AS_5-17-02_P | − |
| 13601936 | 20001556 | At_Drought_Soil_Dry_YF_07-16-03_P | − |
| 12396394 | 20000086 | At_100uM_ABA_Mutants_YF_2-4-02_cDNA_P | − |
| 4909291 | 20000265 | At_Open_Flower_YF_06-19-02_P | − |
| 12711515 | 20000234 | At_Siliques_YF_6-05-02_P | − |
| 12703041 | 20000087 | At_100uM_ABA_Mutants_YF_2-4-02_cDNA_P | − |
| 12703041 | 20000267 | At_Drought_YF_06-25-02_P | − |
| 12670159 | 20001451 | At_Far-red-induction_AM_4-16-03_P | − |
| 12370148 | 20000046 | At_CS237-vs-Columbia_YF_1-24-02_cDNA_P | − |
| 12679922 | 20001558 | At_Drought_Soil_Dry_YF_07-16-03_P | − |
| 12711515 | 108463 | At_Germinating_Seeds_YF_5-22-01_cDNA_P | − |
| 12332135 | 108474 | At_Drought_Flowers_YF_5-23-01_cDNA_P | − |
| 12370148 | 20000437 | At_Drought_YF_06-25-02_P | − |
| 13621692 | 20000173 | At_42deg_Heat_YF_4-11-02_P | − |
| 12396394 | 108435 | At_stm_Mutants_RP_5-2-01_cDNA_P | − |
| 12660077 | 20000439 | At_Roots_YF_7-24-02_P | − |
| 4909291 | 20000234 | At_Siliques_YF_6-05-02_P | − |
| 12396394 | 20000090 | At_2mM_SA_CS3726-Columbia_YF_2-14-02_cDNA_P | − |
| 12660077 | 20000574 | At_100uM_ABA_Mutants_YF_9-9-02_P | − |
| 12669615 | 20000179 | At_Germinating_Seeds_YF_4-11-02_P | − |
| 12396394 | 108589 | At_15mM_NH4NO3_L-to-H_EK_8-24-00_cDNA_P | − |
| 12370148 | 108501 | At_ap2_floral_buds_DJ_7-10-01_cDNA_P | − |
| 13617784 | 108457 | At_Diversity_Expt_RP_5-16-01_cDNA_P | − |
| 4909291 | 20000127 | At_50mM_NH4NO3_L-to-H_Siliques_EK_3-12-02_cDNA_P | − |
| 12455436 | 20000326 | At_Pollen_YF_07-12-02_P | − |
| 12370148 | 108608 | At_100uM_ABA_YF_9-18-01_cDNA_P | − |
| 13612879 | 108667 | At_2mM_SA_YF_11-28-01_cDNA_P | − |
| 12370148 | 20001317 | At_Interploidy_Crosses_RP_04-28-03_P | − |
| 13489977 | 20000171 | At_42deg_Heat_YF_4-11-02_P | − |
| 12370148 | 20000166 | At_100uM_ABA_YF_4-11-02_P | − |
| 13609817 | 20001556 | At_Drought_Soil_Dry_YF_07-16-03_P | − |
| 12679922 | 20001554 | At_Drought_Soil_Dry_YF_07-16-03_P | − |
| 13617784 | 20000456 | At_100uM_BA_YF_7-29-02_P | − |
| 12333534 | 20000088 | At_100uM_ABA_Mutants_YF_2-4-02_cDNA_P | − |
| 4909806 | 20000576 | At_100uM_ABA_Mutants_YF_9-9-02_P | − |
| 4909806 | 20000573 | At_100uM_ABA_Mutants_YF_9-9-02_P | − |
| 4909806 | 20000574 | At_100uM_ABA_Mutants_YF_9-9-02_P | − |
| 12576899 | 20000171 | At_42deg_Heat_YF_4-11-02_P | − |
| 12679922 | 108462 | At_Germinating_Seeds_YF_5-22-01_cDNA_P | − |
| 13621692 | 20000184 | At_Shoots_YF_7-24-02_P | − |
| 12396394 | 20001556 | At_Drought_Soil_Dry_YF_07-16-03_P | − |
| 4909291 | 20000264 | At_Open_Flower_YF_06-19-02_P | − |
| 12322657 | 108577 | At_42deg_Heat_YF_8-24-00_cDNA_P | − |
| 4909806 | 20000575 | At_100uM_ABA_Mutants_YF_9-9-02_P | − |
| 12333534 | 20000070 | At_100uM_ABA_Mutants_YF_2-4-02_cDNA_P | − |
| 13609817 | 108595 | At_Ler-pi_Ovule_RP_8-24-00_cDNA_P | − |
| 12348737 | 108501 | At_ap2_floral_buds_DJ_7-10-01_cDNA_P | − |
| 12396394 | 20000071 | At_100uM_ABA_Mutants_YF_2-4-02_cDNA_P | − |
| 13613553 | 108461 | At_Germinating_Seeds_YF_5-22-01_cDNA_P | − |
| 4909806 | 108607 | At_100uM_ABA_YF_9-18-01_cDNA_P | − |
| 13610584 | 20000267 | At_Drought_YF_06-25-02_P | − |
| 12713856 | 108579 | At_4deg_Cold_YF_8-24-00_cDNA_P | − |
| 13609817 | 20000708 | At_Fis1_Siliques_RP_01-08-03_P | − |
| 12688453 | 20000438 | At_Shoots_YF_7-24-02_P | − |
| 13489977 | 20000506 | At_Wounding_YF_8-19-02_P | − |
| 12348737 | 20000179 | At_Germinating_Seeds_YF_4-11-02_P | − |
| 12711515 | 108579 | At_4deg_Cold_YF_8-24-00_cDNA_P | − |
| 12713856 | 108607 | At_100uM_ABA_YF_9-18-01_cDNA_P | − |
| 12679922 | 108461 | At_Germinating_Seeds_YF_5-22-01_cDNA_P | − |
| 13614841 | 20001451 | At_Far-red-induction_AM_4-16-03_P | − |
| 4909806 | 20000437 | At_Drought_YF_06-25-02_P | − |
| 12325134 | 108569 | At_0.001%_MeJA_YF_8-13-01_cDNA_P | − |
| 4909291 | 20000235 | At_Siliques_YF_6-05-02_P | − |
| 12713856 | 108606 | At_100uM_ABA_YF_9-18-01_cDNA_P | − |
| 12321680 | 20000439 | At_Roots_YF_7-24-02_P | − |
| 13601936 | 20000069 | At_100uM_ABA_Mutants_YF_2-4-02_cDNA_P | − |
| 13610584 | 20000326 | At_Pollen_YF_07-12-02_P | − |
| 12679922 | 108500 | At_DMT-II_YF_7-6-01_cDNA_P | − |
| 12322657 | 20000111 | At_42deg_Heat_YF_2-20-02_cDNA_P | − |
| 13619323 | 20001654 | At_Interploidy_Crosses_RP_04-28-03_P | − |
| 12713856 | 108435 | At_stm_Mutants_RP_5-2-01_cDNA_P | − |
| 12348737 | 108488 | At_50_mM_NH4NO3_L-to-H_Rosette_EK_6-18-01_cDNA_P | − |
| 12576899 | 20001554 | At_Drought_Soil_Dry_YF_07-16-03_P | − |
| 13601936 | 20000438 | At_Shoots_YF_7-24-02_P | − |
| 13614841 | 20000794 | At_Petals_YF_03-06-03_P | − |
| 12455436 | 20000794 | At_Petals_YF_03-06-03_P | − |
| 12333534 | 20001555 | At_Drought_Soil_Dry_YF_07-16-03_P | − |
| 12370148 | 20000308 | At_100mM_NaCl_YF_6-27-02_P | − |
| 12713856 | 108577 | At_42deg_Heat_YF_8-24-00_cDNA_P | − |
| 12711515 | 20000171 | At_42deg_Heat_YF_4-11-02_P | − |
| 12713856 | 20000436 | At_Drought_YF_06-25-02_P | − |
| 12711515 | 20000046 | At_CS237-vs-Columbia_YF_1-24-02_cDNA_P | − |
| 12333534 | 108478 | At_Shoot_Apices_YF_6-5-01_cDNA_P | − |
| 12703041 | 20000090 | At_2mM_SA_CS3726-Columbia_YF_2-14-02_cDNA_P | − |
| 13509244 | 20000184 | At_Shoots_YF_7-24-02_P | − |
| 13610584 | 20000709 | At_15mM_NH4NO3_L-to-H_BH_01-08-03_P | − |
| 12711515 | 20000527 | At_10%_PEG_YF_7-29-02_P | − |
| 12370148 | 108575 | At_Wounding_YF_8-13-01_cDNA_P | − |
| 13612919 | 20001451 | At_Far-red-induction_AM_4-16-03_P | − |
| 12333534 | 20001559 | At_Drought_Soil_Dry_YF_07-16-03_P | − |
| 4905097 | 108461 | At_Germinating_Seeds_YF_5-22-01_cDNA_P | − |
| 12669615 | 20000173 | At_42deg_Heat_YF_4-11-02_P | − |
| 13491988 | 20000451 | At_CS6879_Shoots-Roots_SK_7-29-02_P | − |
| 12669615 | 20001555 | At_Drought_Soil_Dry_YF_07-16-03_P | − |
| 12711515 | 20000111 | At_42deg_Heat_YF_2-20-02_cDNA_P | − |
| 4909291 | 20000213 | At_4deg_Cold_AS_5-17-02_P | − |
| 12333534 | 20000072 | At_100uM_ABA_Mutants_YF_2-4-02_cDNA_P | − |
| 12713856 | 20000437 | At_Drought_YF_06-25-02_P | − |
| 13621692 | 20000265 | At_Open_Flower_YF_06-19-02_P | − |
| 12679922 | 108463 | At_Germinating_Seeds_YF_5-22-01_cDNA_P | − |
| 13491988 | 20001558 | At_Drought_Soil_Dry_YF_07-16-03_P | − |
| 4909806 | 108512 | At_3642-1_RS_7-23-01_cDNA_P | − |
| 12370148 | 108461 | At_Germinating_Seeds_YF_5-22-01_cDNA_P | − |
| 13617784 | 108595 | At_Ler-pi_Ovule_RP_8-24-00_cDNA_P | − |
| 12332135 | 20001397 | At_Line_Comparisons_NA_03-31-03_P | − |
| 12711515 | 20000144 | At_42deg_Heat_YF_2-20-02_cDNA_P | − |
| 4909291 | 108579 | At_4deg_Cold_YF_8-24-00_cDNA_P | − |
| 13610584 | 20000184 | At_Shoots_YF_7-24-02_P | − |
| 12679922 | 20000113 | At_42deg_Heat_YF_2-20-02_cDNA_P | − |
| 12321680 | 20001654 | At_Interploidy_Crosses_RP_04-28-03_P | − |
| 12713856 | 20000286 | At_Open_Flower_YF_06-19-02_P | − |
| 13489977 | 20000227 | At_Root-Tips-vs-Tops_SK_5-30-02_P | − |
| 12711515 | 108464 | At_Germinating_Seeds_YF_5-22-01_cDNA_P | − |
| 12692181 | 108480 | At_Shoot_Apices_YF_6-5-01_cDNA_P | − |
| 12670159 | 20001557 | At_Drought_Soil_Dry_YF_07-16-03_P | − |
| 12370148 | 20000288 | At_Drought_YF_06-25-02_P | − |
| 12670159 | 20000708 | At_Fis1_Siliques_RP_01-08-03_P | − |
| 4905097 | 108474 | At_Drought_Flowers_YF_5-23-01_cDNA_P | − |
| 12396394 | 20000117 | At_100uM_ABA_Mutants_YF_2-4-02_cDNA_P | − |
| 13614559 | 108463 | At_Germinating_Seeds_YF_5-22-01_cDNA_P | − |
| 12670159 | 20001556 | At_Drought_Soil_Dry_YF_07-16-03_P | − |
| 13509244 | 20000326 | At_Pollen_YF_07-12-02_P | − |
| 4996264 | 20000286 | At_Open_Flower_YF_06-19-02_P | − |
| 13614559 | 108461 | At_Germinating_Seeds_YF_5-22-01_cDNA_P | − |
| 12692181 | 108435 | At_stm_Mutants_RP_5-2-01_cDNA_P | − |
| 12333534 | 20000087 | At_100uM_ABA_Mutants_YF_2-4-02_cDNA_P | − |
| 12455436 | 108461 | At_Germinating_Seeds_YF_5-22-01_cDNA_P | − |
| 12670159 | 20000227 | At_Root-Tips-vs-Tops_SK_5-30-02_P | − |
| 12669615 | 20000171 | At_42deg_Heat_YF_4-11-02_P | − |
| 12703041 | 20000451 | At_CS6879_Shoots-Roots_SK_7-29-02_P | − |
| 12333534 | 108561 | At_100uM_ABA_YF_8-9-01_cDNA_P | − |
| 12332135 | 108569 | At_0.001%_MeJA_YF_8-13-01_cDNA_P | − |
| 13621692 | 20000438 | At_Shoots_YF_7-24-02_P | − |
| 12329827 | 20001654 | At_Interploidy_Crosses_RP_04-28-03_P | − |
| 12646933 | 20001397 | At_Line_Comparisons_NA_03-31-03_P | − |
| 12669615 | 20000184 | At_Shoots_YF_7-24-02_P | − |
| 5787483 | 108576 | At_42deg_Heat_YF_8-24-00_cDNA_P | − |
| 12711515 | 20000286 | At_Open_Flower_YF_06-19-02_P | − |
| 12678173 | 20000068 | At_CS3824_vs_Landsberg_YF_1-2802_cDNA_P | − |
| 13612919 | 20001557 | At_Drought_Soil_Dry_YF_07-16-03_P | − |
| 13612919 | 20000090 | At_2mM_SA_CS3726-Columbia_YF_2-14-02_cDNA_P | − |
| 13614559 | 108462 | At_Germinating_Seeds_YF_5-22-01_cDNA_P | − |
| 12333534 | 20000086 | At_100uM_ABA_Mutants_YF_2-4-02_cDNA_P | − |
| 12711515 | 20000265 | At_Open_Flower_YF_06-19-02_P | − |
| 13609100 | 20001248 | At_Far-red-induction_AM_4-16-03_P | − |
| 12370148 | 108579 | At_4deg_Cold_YF_8-24-00_cDNA_P | − |
| 13617784 | 20000269 | At_1mM_KNO3_L-vs-H_Roots_SK_06-27-02_P | − |
| 12735519 | 20000573 | At_100uM_ABA_Mutants_YF_9-9-02_P | − |
| 12646933 | 20000794 | At_Petals_YF_03-06-03_P | − |
| 13489977 | 20000211 | At_0.001%_MeJA_YF_5-17-02_P | − |
| 12713856 | 108608 | At_100uM_ABA_YF_9-18-01_cDNA_P | − |
| 12348737 | 20000173 | At_42deg_Heat_YF_4-11-02_P | − |
| 12711515 | 20000267 | At_Drought_YF_06-25-02_P | − |
| 12703041 | 20001554 | At_Drought_Soil_Dry_YF_07-16-03_P | − |
| 4996264 | 20000264 | At_Open_Flower_YF_06-19-02_P | − |
| 13612919 | 20000437 | At_Drought_YF_06-25-02_P | − |
| 13610584 | 20000268 | At_100mM_NaCl_YF_6-27-02_P | − |
| 13653114 | 20000306 | At_Germinating_Seeds_YF_07-03-02_Sub_P | − |
| 12322657 | 108434 | At_Root_Tips_RP_5-1-01_cDNA_P | − |
| 12703041 | 108457 | At_Diversity_Expt_RP_5-16-01_cDNA_P | − |
| 4909806 | 20001556 | At_Drought_Soil_Dry_YF_07-16-03_P | − |
| 12370148 | 20000506 | At_Wounding_YF_8-19-02_P | − |
| 13489977 | 108434 | At_Root_Tips_RP_5-1-01_cDNA_P | − |
| 13612919 | 108585 | At_5mM_NaNP_YF_8-24-00_cDNA_P | − |
| 12660077 | 20000184 | At_Shoots_YF_7-24-02_P | − |
| 12679922 | 20000179 | At_Germinating_Seeds_YF_4-11-02_P | − |
| 13614559 | 108464 | At_Germinating_Seeds_YF_5-22-01_cDNA_P | − |
| 13610584 | 108574 | At_Wounding_YF_8-13-01_cDNA_P | − |
| 12646933 | 20001449 | At_Line_Comparisons_NA_03-31-03_P | − |
| 13617784 | 20000086 | At_100uM_ABA_Mutants_YF_2-4-02_cDNA_P | − |
| 4909806 | 20001316 | At_Interploidy_Crosses_RP_04-28-03_P | − |
| 12703041 | 20000268 | At_100mM_NaCl_YF_6-27-02_P | − |
| 12576899 | 20001557 | At_Drought_Soil_Dry_YF_07-16-03_P | − |
| 12333534 | 20000093 | At_42deg_Heat_YF_2-20-02_cDNA_P | − |
| 13491988 | 20001560 | At_Drought_Soil_Dry_YF_07-16-03_P | − |
| 4996264 | 20000265 | At_Open_Flower_YF_06-19-02_P | − |
| 12679922 | 20000794 | At_Petals_YF_03-06-03_P | − |
| 4909806 | 20000169 | At_100uM_ABA_YF_4-11-02_P | − |
| 12688453 | 20000573 | At_100uM_ABA_Mutants_YF_9-9-02_P | − |
| 4909806 | 20000234 | At_Siliques_YF_6-05-02_P | − |
| 12711515 | 20000436 | At_Drought_YF_06-25-02_P | − |
| 13619323 | 108500 | At_DMT-II_YF_7-6-01_cDNA_P | − |
| 12713856 | 20001450 | At_Far-red-induction_AM_4-16-03_P | − |
| 12333534 | 20000071 | At_100uM_ABA_Mutants_YF_2-4-02_cDNA_P | − |
| 12348737 | 108594 | At_Ler-rhl_Root_RP_8-24-00_cDNA_P | − |
| 4996264 | 20000184 | At_Shoots_YF_7-24-02_P | − |
| 13609817 | 20000090 | At_2mM_SA_CS3726-Columbia_YF_2-14-02_cDNA_P | − |
| 12321680 | 20000438 | At_Shoots_YF_7-24-02_P | − |
| 12370148 | 20000227 | At_Root-Tips-vs-Tops_SK_5-30-02_P | − |
| 4909291 | 20001558 | At_Drought_Soil_Dry_YF_07-16-03_P | − |
| 4909806 | 20000286 | At_Open_Flower_YF_06-19-02_P | − |
| 12561142 | 20001556 | At_Drought_Soil_Dry_YF_07-16-03_P | − |
| 13617784 | 108499 | At_DMT-II_YF_7-6-01_cDNA_P | − |
| 12321680 | 20001397 | At_Line_Comparisons_NA_03-31-03_P | − |
| 12679922 | 20001449 | At_Line_Comparisons_NA_03-31-03_P | − |
| 13491988 | 108577 | At_42deg_Heat_YF_8-24-00_cDNA_P | − |
| 12333534 | 108607 | At_100uM_ABA_YF_9-18-01_cDNA_P | − |
| 4909291 | 20000214 | At_4deg_Cold_AS_5-17-02_P | − |
| 12396394 | 20001555 | At_Drought_Soil_Dry_YF_07-16-03_P | − |
| 12703041 | 108489 | At_50mM_NH4NO3_L-to-H_Rosette_EK_6-18-01_cDNA_P | − |
| 5787483 | 108577 | At_42deg_Heat_YF_8-24-00_cDNA_P | − |
| 4996264 | 108434 | At_Root_Tips_RP_5-1-01_cDNA_P | − |
| 13612919 | 20000451 | At_CS6879_Shoots-Roots_SK_7-29-02_P | − |
| 12660077 | 20000460 | At_10%_PEG_YF_7-29-02_P | − |
| 13612919 | 20000213 | At_4deg_Cold_AS_5-17-02_P | + |
| 4905097 | 20000113 | At_42deg_Heat_YF_2-20-02_cDNA_P | + |
| 12713856 | 20000066 | At_CS3071_vs_Columbia_YF_1-28-02_cDNA_P | + |
| 12713856 | 108590 | At_15mM_NH4NO3_L-to-H_EK_8-24-00_cDNA_P | + |
| 13489977 | 20000223 | At_CS6632_Shoots-Roots_SK_5-29-02_cDNA_P | + |
| 12576899 | 20000286 | At_Open_Flower_YF_06-19-02_P | + |
| 12333534 | 20001560 | At_Drought_Soil_Dry_YF_07-16-03_P | + |
| 4909291 | 20000496 | At_Guard_Cells_JD_8-13-02_P | + |
| 12713856 | 20001397 | At_Line_Comparisons_NA_03-31-03_P | + |
| 12739224 | 20001558 | At_Drought_Soil_Dry_YF_07-16-03_P | + |
| 4949423 | 20000264 | At_Open_Flower_YF_06-19-02_P | + |
| 12669615 | 20000439 | At_Roots_YF_7-24-02_P | + |
| 13613553 | 20000573 | At_100uM_ABA_Mutants_YF_9-9-02_P | + |
| 12711515 | 108605 | At_100uM_ABA_YF_9-18-01_cDNA_P | + |
| 13614559 | 108583 | At_5mM_H2O2_YF_8-24-00_cDNA_P | + |
| 12656458 | 20001317 | At_Interploidy_Crosses_RP_04-28-03_P | + |
| 13601936 | 108501 | At_ap2_floral_buds_DJ_7-10-01_cDNA_P | + |
| 4906343 | 20001451 | At_Far-red-induction_AM_4-16-03_P | + |
| 13609583 | 20000227 | At_Root-Tips-vs-Tops_SK_5-30-02_P | + |
| 13647840 | 20000234 | At_Siliques_YF_6-05-02_P | + |
| 12669615 | 20001247 | At_Far-red-induction_AM_4-16-03_P | + |
| 13610584 | 108607 | At_100uM_ABA_YF_9-18-01_cDNA_P | + |
| 12660077 | 20001654 | At_Interploidy_Crosses_RP_04-28-03_P | + |
| 13614841 | 20001560 | At_Drought_Soil_Dry_YF_07-16-03_P | + |
| 12713856 | 108591 | At_15mM_NH4NO3_L-to-H_EK_8-24-00_cDNA_P | + |
| 12711515 | 20000086 | At_100uM_ABA_Mutants_YF_2-4-02_cDNA_P | + |
| 12688453 | 20000443 | At_1uM_BR-BRZ_YF_7-24-02_P | + |
| 13614559 | 108668 | At_2mM_SA_YF_11-28-01_cDNA_P | + |
| 4909806 | 20001654 | At_Interploidy_Crosses_RP_04-28-03_P | + |
| 13619323 | 108589 | At_15mM_NH4NO3_L-to-H_EK_8-24-00_cDNA_P | + |
| 12679922 | 20000496 | At_Guard_Cells_JD_8-13-02_P | + |
| 13509244 | 20000286 | At_Open_Flower_YF_06-19-02_P | + |
| 12333534 | 20000184 | At_Shoots_YF_7-24-02_P | + |
| 13619323 | 20000443 | At_1uM_BR-BRZ_YF_7-24-02_P | + |
| 13621692 | 20001247 | At_Far-red-induction_AM_4-16-03_P | + |
| 12713856 | 20000184 | At_Shoots_YF_7-24-02_P | + |
| 13617784 | 108481 | At_Shoot_Apices_YF_6-5-01_cDNA_P | + |
| 12333534 | 20000444 | At_100uM_NAA_YF_7-24-02_P | + |
| 12670159 | 20000575 | At_100uM_ABA_Mutants_YF_9-9-02_P | + |
| 5787483 | 108462 | At_Germinating_Seeds_YF_5-22-01_cDNA_P | + |
| 13614559 | 20000071 | At_100uM_ABA_Mutants_YF_2-4-02_cDNA_P | + |
| 13621692 | 20000573 | At_100uM_ABA_Mutants_YF_9-9-02 P | + |
| 12711515 | 20001504 | At_Far-red-enriched_AM_07-03-03_P | + |
| 12711515 | 108610 | At_100uM_ABA_YF_9-18-01_cDNA_P | + |
| 13613553 | 20000087 | At_100uM_ABA_Mutants_YF_2-4-02_cDNA_P | + |
| 13617784 | 20000127 | At_50mM_NH4NO3_L-to-H_Siliques_EK_3-12-02_cDNA_P | + |
| 12679922 | 20000443 | At_1uM_BR-BRZ_YF_7-24-02_P | + |
| 12735519 | 20000184 | At_Shoots_YF_7-24-02_P | + |
| 5787483 | 108461 | At_Germinating_Seeds_YF_5-22-01_cDNA_P | + |
| 4905097 | 108457 | At_Diversity_Expt_RP_5-16-01_cDNA_P | + |
| 13609817 | 108499 | At_DMT-II_YF_7-6-01_cDNA_P | + |
| 12713856 | 20000438 | At_Shoots_YF_7-24-02_P | + |
| 12678173 | 20000070 | At_100uM_ABA_Mutants_YF_2-4-02_cDNA_P | + |
| 12711515 | 20000574 | At_100uM_ABA_Mutants_YF_9-9-02_P | + |
| 13621692 | 20000179 | At_Germinating_Seeds_YF_4-11-02_P | + |
| 13617784 | 20001555 | At_Drought_Soil_Dry_YF_07-16-03_P | + |
| 13610584 | 20001247 | At_Far-red-induction_AM_4-16-03_P | + |
| 12711515 | 20001316 | At_Interploidy_Crosses_RP_04-28-03_P | + |
| 13614559 | 20000173 | At_42deg_Heat_YF_4-11-02_P | + |
| 12711515 | 108455 | At_20uM_KNO3_H-to-L_SK_5-10-01_cDNA_P | + |
| 13619323 | 108473 | At_Drought_Flowers_YF_5-23-01_cDNA_P | + |
| 12735519 | 20000458 | At_42deg_Heat_YF_7-29-02_P | + |
| 12703041 | 20000113 | At_42deg_Heat_YF_2-20-02_cDNA_P | + |
| 13601936 | 20000264 | At_Open_Flower_YF_06-19-02_P | + |
| 13619323 | 108573 | At_Drought_YF_8-24-00_cDNA_P | + |
| 12713856 | 20000092 | At_42deg_Heat_YF_2-20-02_cDNA_P | + |
| 13610584 | 108462 | At_Germinating_Seeds_YF_5-22-01_cDNA_P | + |
| 12669615 | 20000460 | At_10%_PEG_YF_7-29-02_P | + |
| 13614559 | 20000437 | At_Drought_YF_06-25-02_P | + |
| 12736079 | 20001557 | At_Drought_Soil_Dry_YF_07-16-03_P | + |
| 12735519 | 20000438 | At_Shoots_YF_7-24-02_P | + |
| 13489977 | 108480 | At_Shoot_Apices_YF_6-5-01_cDNA_P | + |
| 12329827 | 108478 | At_Shoot_Apices_YF_6-5-01_cDNA_P | + |
| 13489977 | 20000169 | At_100uM_ABA_YF_4-11-02_P | + |
| 12703041 | 20001654 | At_Interploidy_Crosses_RP_04-28-03_P | + |
| 12348737 | 20000086 | At_100uM_ABA_Mutants_YF_2-4-02_cDNA_P | + |
| 13609583 | 20000234 | At_Siliques_YF_6-05-02_P | + |
| 12455436 | 20001560 | At_Drought_Soil_Dry_YF_07-16-03_P | + |
| 12688453 | 20001504 | At_Far-red-enriched_AM_07-03-03_P | + |
| 13619323 | 108575 | At_Wounding_YF_8-13-01_cDNA_P | + |
| 13612919 | 108461 | At_Germinating_Seeds_YF_5-22-01_cDNA_P | + |
| 13610584 | 108605 | At_100uM_ABA_YF_9-18-01_cDNA_P | + |
| 13617784 | 20000441 | At_1uM_BR-BRZ_YF_7-24-02_P | + |
| 13489977 | 108481 | At_Shoot_Apices_YF_6-5-01_cDNA_P | + |
| 12669615 | 20000169 | At_100uM_ABA_YF_4-11-02_P | + |
| 13612879 | 20000286 | At_Open_Flower_YF_06-19-02_P | + |
| 13489977 | 20000070 | At_100uM_ABA_Mutants_YF_2-4-02_cDNA_P | + |
| 4909291 | 108434 | At_Root_Tips_RP_5-1-01_cDNA_P | + |
| 12688453 | 20000264 | At_Open_Flower_YF_06-19-02_P | + |
| 13613553 | 20000086 | At_100uM_ABA_Mutants_YF_2-4-02_cDNA_P | + |
| 12679922 | 108512 | At_3642-1_RS_7-23-01_cDNA_P | + |
| 13489977 | 20000117 | At_100uM_ABA_Mutants_YF_2-4-02_cDNA_P | + |
| 12711515 | 108607 | At_100uM_ABA_YF_9-18-01_cDNA_P | + |
| 12333534 | 20000457 | At_42deg_Heat_YF_7-29-02_P | + |
| 12711515 | 20000070 | At_100uM_ABA_Mutants_YF_2-4-02_cDNA_P | + |
| 13601936 | 20000458 | At_42deg_Heat_YF_7-29-02_P | + |
| 12348737 | 20000087 | At_100uM_ABA_Mutants_YF_2-4-02_cDNA_P | + |
| 12711515 | 20000495 | At_Guard_Cells_JD_8-13-02_P | + |
| 13609583 | 108461 | At_Germinating_Seeds_YF_5-22-01_cDNA_P | + |
| 12711515 | 20000455 | At_100uM_ABA_YF_4-11-02_P | + |
| 12576899 | 20000227 | At_Root-Tips-vs-Tops_SK_5-30-02_P | + |
| 13619323 | 20000286 | At_Open_Flower_YF_06-19-02_P | + |
| 13491988 | 20001654 | At_Interploidy_Crosses_RP_04-28-03_P | + |
| 13609100 | 20001555 | At_Drought_Soil_Dry_YF_07-16-03_P | + |
| 12688453 | 20001556 | At_Drought_Soil_Dry_YF_07-16-03_P | + |
| 12348737 | 20000227 | At_Root-Tips-vs-Tops_SK_5-30-02_P | + |
| 12660077 | 20000438 | At_Shoots_YF_7-24-02_P | + |
| 12703041 | 108463 | At_Germinating_Seeds_YF_5-22-01_cDNA_P | + |
| 13613553 | 20000436 | At_Drought_YF_06-25-02_P | + |
| 13613553 | 20000794 | At_Petals_YF_03-06-03_P | + |
| 13619323 | 108605 | At_100uM_ABA_YF_9-18-01_cDNA_P | + |
| 12692181 | 108560 | At_100uM_ABA_YF_8-9-01_cDNA_P | + |
| 12679922 | 20000573 | At_100uM_ABA_Mutants_YF_9-9-02_P | + |
| 13617784 | 20001458 | At_50mM_NH4NO3_L-to-H_RS-GM_05-19-03_P | + |
| 4909806 | 108461 | At_Germinating_Seeds_YF_5-22-01_cDNA_P | + |
| 12576899 | 20000496 | At_Guard_Cells_JD_8-13-02_P | + |
| 12692181 | 108590 | At_15mM_NH4NO3_L-to-H_EK_8-24-00_cDNA_P | + |
| 13619323 | 20000070 | At_100uM_ABA_Mutants_YF_2-4-02_cDNA_P | + |
| 13613553 | 108579 | At_4deg_Cold_YF_8-24-00_cDNA_P | + |
| 12711515 | 108512 | At_3642-1_RS_7-23-01_cDNA_P | + |
| 13610584 | 20000573 | At_100uM_ABA_Mutants_YF_9-9-02_P | + |
| 12692181 | 108473 | At_Drought_Flowers_YF_5-23-01_cDNA_P | + |
| 12329827 | 108473 | At_Drought_Flowers_YF_5-23-01_cDNA_P | + |
| 12713856 | 20000457 | At_42deg_Heat_YF_7-29-02_P | + |
| 12711515 | 108501 | At_ap2_floral_buds_DJ_7-10-01_cDNA_P | + |
| 12688453 | 20000234 | At_Siliques_YF_6-05-02_P | + |
| 12679922 | 108594 | At_Ler-rhl_Root_RP_8-24-00_cDNA_P | + |
| 13613553 | 108576 | At_42deg_Heat_YF_8-24-00_cDNA_P | + |
| 12703041 | 108488 | At_50mM_NH4NO3_L-to-H_Rosette_EK_6-18-01_cDNA_P | + |
| 12711515 | 108474 | At_Drought_Flowers_YF_5-23-01_cDNA_P | + |
| 12736079 | 20000185 | At_Roots_YF_7-24-02_P | + |
| 13610584 | 20000574 | At_100uM_ABA_Mutants_YF_9-9-02_P | + |
| 12678173 | 20000236 | At_Siliques_YF_6-05-02_P | + |
| 13612919 | 20000438 | At_Shoots_YF_7-24-02_P | + |
| 13619323 | 20001653 | At_Interploidy_Crosses_RP_04-28-03_P | + |
| 12370148 | 20000069 | At_100uM_ABA_Mutants_YF_2-4-02_cDNA_P | + |
| 12688453 | 20000445 | At_100uM_NAA_YF_7-24-02_P | + |
| 12370148 | 20001458 | At_50mM_NH4NO3_L-to-H_RS-GM_05-19-03_P | + |
| 12739224 | 20001557 | At_Drought_Soil_Dry_YF_07-16-03_P | + |
| 12332135 | 108473 | At_Drought_Flowers_YF_5-23-01_cDNA_P | + |
| 13609817 | 20001654 | At_Interploidy_Crosses_RP_04-28-03_P | + |
| 12370148 | 20001557 | At_Drought_Soil_Dry_YF_07-16-03_P | + |
| 12692181 | 20000355 | At_Siliques_YF_7-18-02_Sub_P | + |
| 12322657 | 20000437 | At_Drought_YF_06-25-02_P | + |
| 12325134 | 108591 | At_15mM_NH4NO3_L-to-H_EK_8-24-00_cDNA_P | + |
| 12713856 | 20001316 | At_Interploidy_Crosses_RP_04-28-03_P | + |
| 12711515 | 20001503 | At_Far-red-enriched_AM_07-03-03_P | + |
| 12688453 | 20000794 | At_Petals_YF_03-06-03_P | + |
| 13617784 | 20001558 | At_Drought_Soil_Dry_YF_07-16-03_P | + |
| 13614559 | 108561 | At_100uM_ABA_YF_8-9-01_cDNA_P | + |
| 12669615 | 20000185 | At_Roots_YF_7-24-02_P | + |
| 13614559 | 20000113 | At_42deg_Heat_YF_2-20-02_cDNA_P | + |
| 4909291 | 108464 | At_Germinating_Seeds_YF_5-22-01_cDNA_P | + |
| 12646933 | 20000264 | At_Open_Flower_YF_06-19-02_P | + |
| 13614841 | 20001247 | At_Far-red-induction_AM_4-16-03_P | + |
| 12455436 | 108488 | At_50mM_NH4NO3_L-to-H_Rosette_EK_6-18-01_cDNA_P | + |
| 12703041 | 108572 | At_Drought_YF_8-24-00_cDNA_P | + |
| 13614559 | 20000794 | At_Petals_YF_03-06-03_P | + |
| 13610584 | 108668 | At_2mM_SA_YF_11-28-01_cDNA_P | + |
| 13612879 | 108590 | At_15mM_NH4NO3_L-to-H_EK_8-24-00_cDNA_P | + |
| 12396394 | 108464 | At_Germinating_Seeds_YF_5-22-01_cDNA_P | + |
| 12370148 | 108584 | At_5mM_NaNP_YF_8-24-00_cDNA_P | + |
| 12660077 | 20000265 | At_Open_Flower_YF_06-19-02_P | + |
| 13614559 | 108573 | At_Drought_YF_8-24-00_cDNA_P | + |
| 12396394 | 108462 | At_Germinating_Seeds_YF_5-22-01_cDNA_P | + |
| 12332135 | 20000443 | At_1uM_BR-BRZ_YF_7-24-02_P | + |
| 13617784 | 20001560 | At_Drought_Soil_Dry_YF_07-16-03_P | + |
| 13613553 | 20000113 | At_42deg_Heat_YF_2-20-02_cDNA_P | + |
| 12711515 | 20000573 | At_100uM_ABA_Mutants_YF_9-9-02_P | + |
| 12692181 | 108573 | At_Drought_YF_8-24-00_cDNA_P | + |
| 12370148 | 108463 | At_Germinating_Seeds_YF_5-22-01_cDNA_P | + |
| 12333534 | 20000244 | At_Caf_Knockout_RS_6-13-02_P | + |
| 13489977 | 20000443 | At_1uM_BR-BRZ_YF_7-24-02_P | + |
| 12713856 | 20000180 | At_Germinating_Seeds_YF_4-11-02_P | + |
| 12370148 | 108588 | At_15mM_NH4NO3_L-to-H_EK_8-24-00_cDNA_P | + |
| 13614559 | 20000458 | At_42deg_Heat_YF_7-29-02_P | + |
| 4909806 | 20000527 | At_10%_PEG_YF_7-29-02_P | + |
| 13609583 | 20000180 | At_Germinating_Seeds_YF_4-11-02_P | + |
| 12678173 | 20000234 | At_Siliques_YF_6-05-02_P | + |
| 4909806 | 108573 | At_Drought_YF_8-24-00_cDNA_P | + |
| 12711515 | 20000227 | At_Root-Tips-vs-Tops_SK_5-30-02_P | + |
| 12348737 | 20000090 | At_2mM_SA_CS3726-Columbia_YF_2-14-02_cDNA_P | + |
| 12576899 | 20000264 | At_Open_Flower_YF_06-19-02_P | + |
| 13613553 | 108595 | At_Ler-pi_Ovule_RP_8-24-00_cDNA_P | + |
| 12713856 | 108489 | At_50mM_NH4NO3_L-to-H_Rosette_EK_6-18-01_cDNA_P | + |
| 12688453 | 20000308 | At_100mM_NaCl_YF_6-27-02_P | + |
| 13619323 | 20000086 | At_100uM_ABA_Mutants_YF_2-4-02_cDNA_P | + |
| 12692181 | 108462 | At_Germinating_Seeds_YF_5-22-01_cDNA_P | + |
| 12678173 | 20000574 | At_100uM_ABA_Mutants_YF_9-9-02_P | + |
| 12348737 | 20000088 | At_100uM_ABA_Mutants_YF_2-4-02_cDNA_P | + |
| 12348737 | 108667 | At_2mM_SA_YF_11-28-01_cDNA_P | + |
| 12333534 | 20001558 | At_Drought_Soil_Dry_YF_07-16-03_P | + |
| 12692181 | 108575 | At_Wounding_YF_8-13-01_cDNA_P | + |
| 13609817 | 20000185 | At_Roots_YF_7-24-02_P | + |
| 12688453 | 20000495 | At_Guard_Cells_JD_8-13-02_P | + |
| 12348737 | 20000117 | At_100uM_ABA_Mutants_YF_2-4-02_cDNA_P | + |
| 13617784 | 20000113 | At_42deg_Heat_YF_2-20-02_cDNA_P | + |
| 12329827 | 20000437 | At_Drought_YF_06-25-02_P | + |
| 12329827 | 108590 | At_15mM_NH4NO3_L-to-H_EK_8-24-00_cDNA_P | + |
| 12348737 | 20001560 | At_Drought_Soil_Dry_YF_07-16-03_P | + |
| 12711515 | 108561 | At_100uM_ABA_YF_8-9-01_cDNA_P | + |
| 13612879 | 20000264 | At_Open_Flower_YF_06-19-02_P | + |
| 13619323 | 20000069 | At_100uM_ABA_Mutants_YF_2-4-02_cDNA_P | + |
| 13609817 | 20000439 | At_Roots_YF_7-24-02_P | + |
| 13614841 | 20000213 | At_4deg_Cold_AS_5-17-02_P | + |
| 4909806 | 20000438 | At_Shoots_YF_7-24-02_P | + |
| 12348737 | 20000709 | At_15mM_NH4NO3_L-to-H_BH_01-08-03_P | + |
| 13489977 | 108584 | At_5mM_NaNP_YF_8-24-00_cDNA_P | + |
| 12332135 | 108457 | At_Diversity_Expt_RP_5-16-01_cDNA_P | + |
| 12370148 | 108470 | At_2mM_SA_CS3726-Columbia_YF_5-23-01 cDNA_P | + |
| 12736079 | 20000439 | At_Roots_YF_7-24-02_P | + |
| 12713856 | 108463 | At_Germinating_Seeds_YF_5-22-01_cDNA_P | + |
| 12396394 | 20000438 | At_Shoots_YF_7-24-02_P | + |
| 12711515 | 20001560 | At_Drought_Soil_Dry_YF_07-16-03_P | + |
| 13609817 | 108573 | At_Drought_YF_8-24-00_cDNA_P | + |
| 12370148 | 20000527 | At_10%_PEG_YF_7-29-02_P | + |
| 13491988 | 108572 | At_Drought_YF_8-24-00_cDNA_P | + |
| 12688453 | 20000437 | At_Drought_YF_06-25-02_P | + |
| 12688453 | 20000267 | At_Drought_YF_06-25-02_P | + |
| 12322657 | 20000213 | At_4deg_Cold_AS_5-17-02_P | + |
| 4909291 | 20000113 | At_42deg_Heat_YF_2-20-02_cDNA_P | + |
| 13489977 | 20001247 | At_Far-red-induction_AM_4-16-03_P | + |
| 12692181 | 20000046 | At_CS237-vs-Columbia_YF_1-24-02_cDNA_P | + |
| 12692181 | 20000352 | At_Drought_YF_07-18-02_Sub_P | + |
| 12735519 | 20000265 | At_Open_Flower_YF_06-19-02_P | + |
| 12679922 | 20000441 | At_1uM_BR-BRZ_YF_7-24-02_P | + |
| 12678173 | 20000794 | At_Petals_YF_03-06-03_P | + |
| 12688453 | 20001560 | At_Drought_Soil_Dry_YF_07-16-03_P | + |
| 12660077 | 20000286 | At_Open_Flower_YF_06-19-02_P | + |
| 12646933 | 20000708 | At_Fis1_Siliques_RP_01-08-03_P | + |
| 12688453 | 20001316 | At_Interploidy_Crosses_RP_04-28-03_P | + |
| 12711515 | 20000169 | At_100uM_ABA_YF_4-11-02_P | + |
| 13647840 | 20000286 | At_Open_Flower_YF_06-19-02_P | + |
| 13617784 | 108573 | At_Drought_YF_8-24-00_cDNA_P | + |
| 13614841 | 20001558 | At_Drought_Soil_Dry_YF_07-16-03_P | + |
| 12678173 | 20000573 | At_100uM_ABA_Mutants_YF_9-9-02_P | + |
| 12370148 | 108454 | At_20uM_KNO3_H-to-L_SK_5-10-01_cDNA_P | + |
| 12322657 | 108579 | At_4deg_Cold_YF_8-24-00_cDNA_P | + |
| 12688453 | 20001554 | At_Drought_Soil_Dry_YF_07-16-03_P | + |
| 13613553 | 20000111 | At_42deg_Heat_YF_2-20-02_cDNA_P | + |
| 12333534 | 20001247 | At_Far-red-induction_AM_4-16-03_P | + |
| 12661844 | 20000708 | At_Fis1_Siliques_RP_01-08-03_P | + |
| 12332135 | 108501 | At_ap2_floral_buds_DJ_7-10-01_cDNA_P | + |
| 13491988 | 20000496 | At_Guard_Cells_JD_8-13-02_P | + |
| 13491988 | 20000179 | At_Germinating_Seeds_YF_4-11-02_P | + |
| 12348737 | 20000071 | At_100uM_ABA_Mutants_YF_2-4-02_cDNA_P | + |
| 13609583 | 20000235 | At_Siliques_YF_6-05-02_P | + |
| 12688453 | 20001555 | At_Drought_Soil_Dry_YF_07-16-03_P | + |
| 12348737 | 108469 | At_2mM_SA_CS3726-Columbia_YF_5-23-01_cDNA_P | + |
| 4909291 | 108585 | At_5mM_NaNP_YF_8-24-00_cDNA_P | + |
| 13613553 | 20000173 | At_42deg_Heat_YF_4-11-02_P | + |
| 12646933 | 20000286 | At_Open_Flower_YF_06-19-02_P | + |
| 13489977 | 108455 | At_20uM_KNO3_H-to-L_SK_5-10-01_cDNA_P | + |
| 12678173 | 20000265 | At_Open_Flower_YF_06-19-02_P | + |
| 12370148 | 20001248 | At_Far-red-induction_AM_4-16-03_P | + |
| 12679922 | 108480 | At_Shoot_Apices_YF_6-5-01_cDNA_P | + |
| 13612879 | 108573 | At_Drought_YF_8-24-00_cDNA_P | + |
| 12678173 | 20000235 | At_Siliques_YF_6-05-02_P | + |
| 13617784 | 20000709 | At_15mM_NH4NO3_L-to-H_BH_01-08-03_P | + |
| 13601936 | 20000111 | At_42deg_Heat_YF_2-20-02_cDNA_P | + |
| 13489977 | 108454 | At_20uM_KNO3_H-to-L_SK_5-10-01_cDNA_P | + |
| 4996264 | 108457 | At_Diversity_Expt_RP_5-16-01_cDNA_P | + |
| 13619323 | 20000071 | At_100uM_ABA_Mutants_YF_2-4-02_cDNA_P | + |
| 12348737 | 20000089 | At_2mM_SA_CS3726-Columbia_YF_2-14-02_cDNA_P | + |
| 12370148 | 20000460 | At_10%_PEG_YF_7-29-02_P | + |
| 13617784 | 20001557 | At_Drought_Soil_Dry_YF_07-16-03_P | + |
| 12348737 | 20000072 | At_100uM_ABA_Mutants_YF_2-4-02_cDNA_P | + |
| 13613553 | 108573 | At_Drought_YF_8-24-00_cDNA_P | + |
| 12735519 | 20001653 | At_Interploidy_Crosses_RP_04-28-03_P | + |
| 12348737 | 20001247 | At_Far-red-induction_AM_4-16-03_P | + |
| 12329827 | 108589 | At_15mM_NH4NO3_L-to-H_EK_8-24-00_cDNA_P | + |
| 12321680 | 108589 | At_15mM_NH4NO3_L-to-H_EK_8-24-00_cDNA_P | + |
| 13613553 | 20000072 | At_100uM_ABA_Mutants_YF_2-4-02_cDNA_P | + |
| 4909806 | 108463 | At_Germinating_Seeds_YF_5-22-01_cDNA_P | + |
| 12688453 | 20000268 | At_100mM_NaCl | + |
| 13489977 | 20000527 | At_10%_PEG | + |
| 4909291 | 20000458 | At_42deg_Heat | + |
| 12325134 | 108590 | At_15mM_NH4NO3_L-to-H_cDNA_P | + |
| 12322657 | 20000088 | At_100uM_ABA_Mutants_cDNA_P | + |
| 4909291 | 108577 | At_42deg_Heat_cDNA_P | + |
| 12321680 | 20000264 | At_Open_Flower | + |
| 13619323 | 20000072 | At_100uM_ABA_Mutants_cDNA_P | + |
| 12646933 | 108590 | At_15mM_NH4NO3_L-to-H_cDNA_P | + |
| 13610584 | 20001451 | At_Far-red-induction | + |
| 12370148 | 20001308 | At_Line_Comparisons | + |
| 4909291 | 20000173 | At_42deg_Heat | + |
| 12692181 | 20000087 | At_100uM_ABA_Mutants_cDNA_P | + |
| 13610584 | 20000180 | At_Germinating_Seeds | + |
| 12713856 | 108462 | At_Germinating_Seeds_cDNA_P | + |
| 12333534 | 20001451 | At_Far-red-induction | + |
| 12669615 | 20000455 | At_100uM_ABA | + |
| 4906343 | 20000264 | At_Open_Flower | + |
| 13610584 | 20000527 | At_10%_PEG | + |
| 12713856 | 20000495 | At_Guard_Cells | + |
| 12348737 | 20000069 | At_100uM_ABA_Mutants_cDNA_P | + |
| 13613553 | 108668 | At_2mM_SA_cDNA_P | + |
| 13619323 | 20000264 | At_Open_Flower | + |
| 12692181 | 108463 | At_Germinating_Seeds_cDNA_P | + |
| 13610584 | 108457 | At_Diversity_Expt_cDNA_P | + |
| 13612879 | 20000453 | At_100uM_ABA | + |
| 12669615 | 20000453 | At_100uM_ABA | + |
| 13491988 | 20000180 | At_Germinating_Seeds | + |
| 13613553 | 20000458 | At_42deg_Heat | + |
| 12711515 | 20000071 | At_100uM_ABA_Mutants_cDNA_P | + |
| 12670159 | 20000495 | At_Guard_Cells | + |
| 13489977 | 20000441 | At_1uM_BR-BRZ | + |
| 12370148 | 20000441 | At_1uM_BR-BRZ | + |
| 12646933 | 108591 | At_15mM_NH4NO3_L-to-H_cDNA_P | + |
| 12348737 | 20000070 | At_100uM_ABA_Mutants_cDNA_P | + |
| 13489977 | 20000069 | At_100uM_ABA_Mutants_cDNA_P | + |
| 12348737 | 108470 | At_2mM_SA_CS3726-Columbia_cDNA_P | + |
| 13610584 | 108464 | At_Germinating_Seeds_cDNA_P | + |
| 13613553 | 20001557 | At_Drought_Soil_Dry | + |
| 13614841 | 20001557 | At_Drought_Soil_Dry | + |
| 12370148 | 20000443 | At_1uM_BR-BRZ | + |
| 12322657 | 20000072 | At_100uM_ABA_Mutants_cDNA_P | + |
| 12661844 | 20000236 | At_Siliques | + |
| 4905097 | 20000236 | At_Siliques | + |
| 12711515 | 20000117 | At_100uM_ABA_Mutants_cDNA_P | + |
| 12703041 | 108462 | At_Germinating_Seeds_cDNA_P | + |
| 12322657 | 20000086 | At_100uM_ABA_Mutants_cDNA_P | + |
| 12370148 | 108481 | At_Shoot_Apices_cDNA_P | + |
| 12703041 | 108573 | At_Drought_cDNA_P | + |
| 12692181 | 108608 | At_100uM_ABA_cDNA_P | + |
| 12321680 | 108591 | At_15mM_NH4NO3_L-to-H_cDNA_P | + |
| 12692181 | 108464 | At_Germinating_Seeds_cDNA_P | + |
| 4909291 | 20000111 | At_42deg_Heat_cDNA_P | + |
| 4909806 | 108464 | At_Germinating_Seeds_cDNA_P | + |
| 13613553 | 20000709 | At_15mM_NH4NO3_L-to-H | + |
| 12692181 | 108572 | At_Drought_cDNA_P | + |
| 4909806 | 20000184 | At_Shoots | + |
| 13612879 | 20000234 | At_Siliques | + |
| 4949423 | 20000437 | At_Drought | + |
| 4905097 | 20000235 | At_Siliques | + |
| 12692181 | 20000089 | At_2mM_SA_CS3726-Columbia_cDNA_P | + |
| 12711515 | 108609 | At_100uM_ABA_cDNA_P | + |
| 12688453 | 20000436 | At_Drought | + |
| 13647840 | 20000794 | At_Petals | + |
| 12322657 | 20000090 | At_2mM_SA_CS3726-Columbia_cDNA_P | + |
| 13489977 | 108595 | At_Ler-pi_Ovule_cDNA_P | + |
| 13617784 | 108591 | At_15mM_NH4NO3_L-to-H_cDNA_P | + |
| 4909806 | 108462 | At_Germinating_Seeds_cDNA_P | + |
| 12664333 | 20001451 | At_Far-red-induction | + |
| 12333534 | 20000495 | At_Guard_Cells | + |
| 13612919 | 20000180 | At_Germinating_Seeds | + |
| 13609817 | 108463 | At_Germinating_Seeds_cDNA_P | + |
| 12396394 | 108573 | At_Drought_cDNA_P | + |
| 12332135 | 20000264 | At_Open_Flower | + |
| 12321680 | 20000236 | At_Siliques_YF | + |
| 13647840 | 20000264 | At_Open_Flower | + |
| 4909806 | 20000708 | At_Fis1_Siliques | + |
| 13612879 | 20000708 | At_Fis1_Siliques | + |
| 12332135 | 108589 | At_15mM_NH4NO3_L-to-H_cDNA_P | + |
| 12692181 | 108501 | At_ap2_floral_buds_cDNA_P | + |
| 12678173 | 20000264 | At_Open_Flower | + |
| 13617784 | 20000458 | At_42deg_Heat | + |
| 12329827 | 20000180 | At_Germinating_Seeds | + |
| 13619323 | 20000234 | At_Siliques | + |
| 12348737 | 20001654 | At_Interploidy_Crosses | + |
| 12329827 | 108591 | At_15mM_NH4NO3_L-to-H_cDNA_P | + |
| 12688453 | 20000453 | At_100uM_ABA | + |
| 12713856 | 108464 | At_Germinating_Seeds_cDNA_P | + |
| 13491988 | 108573 | At_Drought | + |
| 12396394 | 20000184 | At_Shoots | + |
| 12348737 | 108572 | At_Drought_cDNA_P | + |
| 12348737 | 108457 | At_Diversity_Expt_cDNA_P | + |
| 13610584 | 20001450 | At_Far-red-induction | + |
| 12660077 | 20000794 | At_Petals | + |
| 13619323 | 108591 | At_15mM_NH4NO3_L-to-H_cDNA_P | + |
| 12692181 | 20000072 | At_100uM_ABA_Mutants_cDNA_P | + |
| 4905097 | 20000708 | At_Fis1_Siliques | + |
| 12321680 | 20000235 | At_Siliques | + |
| 12703041 | 108464 | At_Germinating_Seeds_cDNA_P | + |
| 12661844 | 20000235 | At_SiliquesP | + |
| 12348737 | 108585 | At_5mM_NaNP_cDNA_P | + |
| 13612919 | 108462 | At_Germinating_Seeds_cDNA_P | + |
| 12678173 | 20000286 | At_Open_Flower | + |
| 4905097 | 20000234 | At_Siliques | + |
| 12321680 | 20000234 | At_Siliques | + |
| 13489977 | 20001308 | At_Line_Comparisons | + |
| 12692181 | 20000086 | At_100uM_ABA_MutantscDNA_P | + |
| 12455436 | 108434 | At_Root_TipscDNA_P | + |
| 13617784 | 108588 | At_15mM_NH4NO3_L-to-H_cDNA_P | + |
| 12322657 | 20000070 | At_100uM_ABA_Mutants_cDNA_P | + |
| 13614559 | 108585 | At_5mM_NaNP_cDNA_P | + |
| 13610584 | 20001248 | At_Far-red-induction | + |
| 12711515 | 20000441 | At_1uM_BR-BRZ | + |
| 12329827 | 20000234 | At_Siliques | + |
| 12692181 | 20000069 | At_100uM_ABA_Mutants_cDNA_P | + |
| 13619323 | 20000708 | At_Fis1_Siliques | + |
| 12322657 | 20001248 | At_Far-red-induction | + |
| 12332135 | 20000235 | At_Siliques | + |
| 12692181 | 20000349 | At_100uM_ABA_{—} | + |
| 13621692 | 20000326 | At_Pollen | + |
| 13489977 | 20000460 | At_10%_PEG | + |
| 12576899 | 20000794 | At_Petals | + |
| 12711515 | 20000453 | At_100uM_ABA | + |
| 12329827 | 20000179 | At_Germinating_Seeds | + |
| 12322657 | 20000071 | At_100uM_ABA_Mutants_cDNA_P | + |
| 12692181 | 108606 | At_100uM_ABA_cDNA_P | + |
| 12329827 | 20000708 | At_Fis1_Siliques | + |
| 4909291 | 108576 | At_42deg_Heat_cDNA_P | + |
| 12678173 | 20000326 | At_Pollen | + |
| 12348737 | 108668 | At_2mM_SA_cDNA_P | + |
| 12670159 | 20000451 | At_CS6879_Shoots-Roots | + |
| 12692181 | 20000071 | At_100uM_ABA_Mutants_cDNA_P | + |
| 12711515 | 108480 | At_Shoot_Apices_cDNA_P | + |
| 12332135 | 20000236 | At_Siliques | + |
| 13609817 | 108464 | At_Germinating_Seeds_cDNA_P | + |
| 12348737 | 108573 | At_Drought_cDNA_P | + |
| 12692181 | 108610 | At_100uM_ABA_cDNA_P | + |
| 12322657 | 20000117 | At_100uM_ABA_Mutants_cDNA_P | + |
| 12321680 | 20000708 | At_Fis1_Siliques | + |
| 12646933 | 20000235 | At_Siliques | + |
| 12325134 | 108501 | At_ap2_floral_buds_cDNA_P | + |
| 12711515 | 20001451 | At_Far-red-induction | + |
| 13609817 | 108462 | At_Germinating_Seeds_cDNA_P | + |
| 12661844 | 20000234 | At_Siliques | + |
| 13613553 | 108585 | At_5mM_NaNP_cDNA_P | + |
| 12692181 | 108561 | At_100uM_ABA_cDNA_P | + |
| 13609100 | 20000709 | At_15mM_NH4NO3_L-to-H_{—} | + |
| 4909291 | 20000112 | At_42deg_Heat_cDNA_P | + |
| 12711515 | 108481 | At_Shoot_Apices_cDNA_P | + |
| 12692181 | 108609 | At_100uM_ABA_cDNA_P | + |
| 12679922 | 20001247 | At_Far-red-induction | + |
| 12646933 | 20000236 | At_Siliques | + |
| 12646933 | 20000234 | At_Siliques | + |
| 12322657 | 20000087 | At_100uM_ABA_Mutants_cDNA_P | + |
| 12679922 | 20001248 | At_Far-red-induction_{—} | + |
| 12692181 | 20000088 | At_100uM_ABA_Mutants_cDNA_P | + |
| 12646933 | 108501 | At_ap2_floral_buds_cDNA_P | + |
| 4909806 | 108488 | At_50mM_NH4NO3_L-to-H_Rosette_cDNA_P | + |
| 12692181 | 20000070 | At_100uM_ABA_Mutants_cDNA_P | + |
| 4909291 | 20000171 | At_42deg_Heat | + |
| 12396394 | 108572 | At_Drought_cDNA_P | + |
| 12692181 | 108512 | At_3642-1_cDNA_P | + |
| 12322657 | 20000069 | At_100uM_ABA_Mutants_cDNA_P | + |
| 12329827 | 20000236 | At_Siliques | + |
| 13647840 | 20000326 | At_Pollen | + |
| 12332135 | 20000234 | At_Siliques | + |
| 12679922 | 20001450 | At_Far-red-induction | + |
| 12711515 | 20001248 | At_Far-red-induction | + |
| 4909291 | 20000093 | At_42deg_Heat_cDNA_P | + |
| 12692181 | 108605 | At_100uM_ABA_cDNA_P | + |
| 12692181 | 108607 | At_100uM_ABA_cDNA_P | + |
| 4909291 | 20000144 | At_42deg_Heat_cDNA_P | + |
| 13619323 | 108595 | At_Ler-pi_Ovule_cDNA_P | + |
| 12692181 | 20000117 | At_100uM_ABA_Mutants_cDNA_P | + |
| 12321680 | 108501 | At_ap2_floral_buds_cDNA_P | + |
| 12679922 | 20001451 | At_Far-red-induction | + |
| 12711515 | 20001450 | At_Far-red-induction | + |
| 13612879 | 108595 | At_Ler-pi_Ovule_cDNA_P | + |
| 13609100 | 20000171 | At_42deg_Heat | + |
| 13609100 | 20000173 | At_42deg_Heat | + |
| 12692181 | 108595 | At_Ler-pi_Ovule | + |
| 4949423 | 108595 | At_Ler-pi_Ovule | + |
| 13609100 | 20000458 | At_42deg_Heat | + |
| 12329827 | 108595 | At_Ler-pi_Ovule | + |
| 12332135 | 108595 | At_Ler-pi_Ovule | + |
| 12321680 | 108595 | At_Ler-pi_Ovule | + |
| 12370095 | 108501 | At_ap2_floral_buds_cDNA_P | + |
| 12370095 | 108584 | At_5mM_NaNP_cDNA_P | + |
| 12370095 | 108589 | At_15mM_NH4NO3_L-to-H_cDNA_P | + |
| 12370095 | 108590 | At_15mM_NH4NO3_L-to-H_cDNA_P | + |
| 12370095 | 108591 | At_15mM_NH4NO3_L-to-H_cDNA_P | + |
| 12370095 | 108595 | At_Ler-pi_Ovule_cDNA_P | + |
| 12370095 | 20000234 | At_Siliques_P | + |
| 12370095 | 20000235 | At_Siliques_P | + |
| 12370095 | 20000264 | At_Open_Flower_P | + |
| 12385291 | 108434 | At_Root_Tips_cDNA_P | + |
| 12385291 | 108470 | At_2mM_SA_CS3726-Columbia_cDNA_P | + |
| 12385291 | 108572 | At_Drought_cDNA_P | + |
| 12385291 | 108573 | At_Drought_cDNA_P | + |
| 12385291 | 108574 | At_Wounding_cDNA_P | + |
| 12385291 | 20000184 | At_Shoots_P | + |
| 12385291 | 20000236 | At_Siliques_P | + |
| 12385291 | 20000244 | At_Caf_Knockout_P | + |
| 12385291 | 20000268 | At_100mM_NaCl_P | + |
| 12385291 | 20000456 | At_100uM_BA_P | + |
| 12385291 | 20000496 | At_Guard_Cells_P | + |
| 12385291 | 20001557 | At_Drought_Soil_Dry_P | + |
| 12385291 | 20001558 | At_Drought_Soil_Dry_P | + |
| 12385291 | 20001560 | At_Drought_Soil_Dry_P | + |
| 12385291 | 20001757 | At_50mM_NH4NO3_L-to-H_P | + |
| 12395532 | 108454 | At_20uM_KNO3_H-to-L_cDNA_P | + |
| 12395532 | 108455 | At_20uM_KNO3_H-to-L_cDNA_P | + |
| 12395532 | 108470 | At_2mM_SA_CS3726-Columbia_cDNA_P | + |
| 12395532 | 108480 | At_Shoot_Apices_cDNA_P | + |
| 12395532 | 108481 | At_Shoot_Apices_cDNA_P | + |
| 12395532 | 108574 | At_Wounding_cDNA_P | + |
| 12395532 | 108578 | At_4deg_Cold_cDNA_P | + |
| 12395532 | 108579 | At_4deg_Cold_cDNA_P | + |
| 12395532 | 108584 | At_5mM_NaNP_cDNA_P | + |
| 12395532 | 108585 | At_5mM_NaNP_cDNA_P | + |
| 12395532 | 108588 | At_15mM_NH4NO3_L-to-H_cDNA_P | + |
| 12395532 | 108667 | At_2mM_SA_cDNA_P | + |
| 12395532 | 20000213 | At_4deg_Cold_P | + |
| 12395532 | 20000214 | At_4deg_Cold_P | + |
| 12395532 | 20000441 | At_1uM_BR-BRZ_P | + |
| 12395532 | 20000443 | At_1uM_BR-BRZ_P | + |
| 12395532 | 20000460 | At_10%_PEG_P | + |
| 12395532 | 20000527 | At_10%_PEG_P | + |
| 12395532 | 20001247 | At_Far-red-induction_P | + |
| 12395532 | 20001308 | At_Line_Comparisons_P | + |
| 12395532 | 20001458 | At_50mM_NH4NO3_L-to-H_P | + |
| 12395532 | 20001557 | At_Drought_Soil_Dry_P | + |
| 12395532 | 20001558 | At_Drought_Soil_Dry_P | + |
| 12395532 | 20001560 | At_Drought_Soil_Dry_P | + |
| 12575820 | 108573 | At_Drought_cDNA_P | + |
| 12575820 | 108595 | At_Ler-pi_Ovule_cDNA_P | + |
| 12575820 | 20000112 | At_42deg_Heat_cDNA_P | + |
| 12575820 | 20000179 | At_Germinating_Seeds_P | + |
| 12575820 | 20000180 | At_Germinating_Seeds_P | + |
| 12575820 | 20000438 | At_Shoots_P | + |
| 12600234 | 20000234 | At_Siliques_P | + |
| 12600234 | 20000458 | At_42deg_Heat_P | + |
| 12600234 | 20001556 | At_Drought_Soil_Dry_P | + |
| 12600234 | 20001557 | At_Drought_Soil_Dry_P | + |
| 12600234 | 20001558 | At_Drought_Soil_Dry_P | + |
| 12600234 | 20001756 | At_50mM_NH4NO3_L-to-H_P | + |
| 12600234 | 20001757 | At_50mM_NH4NO3_L-to-H_P | + |
| 12603755 | 20000185 | At_Roots_P | + |
| 12603755 | 20000234 | At_Siliques_P | + |
| 12603755 | 20000439 | At_Roots_P | + |
| 12603755 | 20000495 | At_Guard_Cells_P | + |
| 12603755 | 20000496 | At_Guard_Cells_P | + |
| 12640578 | 108461 | At_Germinating_Seeds_cDNA_P | + |
| 12640578 | 20000180 | At_Germinating_Seeds_P | + |
| 12640578 | 20000495 | At_Guard_Cells_P | + |
| 12640578 | 20001247 | At_Far-red-induction_P | + |
| 12640578 | 20001248 | At_Far-red-induction_P | + |
| 12640578 | 20001450 | At_Far-red-induction_P | + |
| 12647555 | 108455 | At_20uM_KNO3_H-to-L_cDNA_P | + |
| 12647555 | 20000179 | At_Germinating_Seeds_P | + |
| 12647555 | 20000180 | At_Germinating_Seeds_P | + |
| 12647555 | 20000227 | At_Root-Tips-vs-Tops_P | + |
| 12647555 | 20000496 | At_Guard_Cells_P | + |
| 12647555 | 20001557 | At_Drought_Soil_Dry_P | + |
| 12647555 | 20001558 | At_Drought_Soil_Dry_P | + |
| 12647555 | 20001560 | At_Drought_Soil_Dry_P | + |
| 12649228 | 108573 | At_Drought_cDNA_P | + |
| 12649228 | 20000495 | At_Guard_Cells_P | + |
| 12721583 | 20000496 | At_Guard_Cells_P | + |
| 12721583 | 20001248 | At_Far-red-induction_P | + |
| 12721583 | 20001450 | At_Far-red-induction_P | + |
| 13612380 | 108454 | At_20uM_KNO3_H-to-L_cDNA_P | + |
| 13612380 | 108464 | At_Germinating_Seeds_cDNA_P | + |
| 13612380 | 108480 | At_Shoot_Apices_cDNA_P | + |
| 13612380 | 108568 | At_0.001%_MeJA_cDNA_P | + |
| 13612380 | 108574 | At_Wounding_cDNA_P | + |
| 13612380 | 108584 | At_5mM_NaNP_cDNA_P | + |
| 13612380 | 108585 | At_5mM_NaNP_cDNA_P | + |
| 13612380 | 108588 | At_15mM_NH4NO3_L-to-H_cDNA_P | + |
| 13612380 | 108594 | At_Ler-rhl_Root_cDNA_P | + |
| 13612380 | 108595 | At_Ler-pi_Ovule_cDNA_P | + |
| 13612380 | 108667 | At_2mM_SA_cDNA_P | + |
| 13612380 | 20000069 | At_100uM_ABA_Mutants_cDNA_P | + |
| 13612380 | 20000086 | At_100uM_ABA_Mutants_cDNA_P | + |
| 13612380 | 20000090 | At_2mM_SA_CS3726-Columbia_cDNA_P | + |
| 13612380 | 20000213 | At_4deg_Cold_P | + |
| 13612380 | 20000441 | At_1uM_BR-BRZ_P | + |
| 13612380 | 20000443 | At_1uM_BR-BRZ_P | + |
| 13612380 | 20001248 | At_Far-red-induction_P | + |
| 12370095 | 20000184 | At_Shoots_P | − |
| 12370095 | 20000185 | At_Roots_P | − |
| 12370095 | 20000245 | At_Caf_Knockout_P | − |
| 12370095 | 20000438 | At_Shoots_P | − |
| 12370095 | 20000439 | At_Roots_P | − |
| 12370095 | 20000794 | At_Petals_P | − |
| 12385291 | 108461 | At_Germinating_Seeds_cDNA_P | − |
| 12385291 | 108462 | At_Germinating_Seeds_cDNA_P | − |
| 12385291 | 108463 | At_Germinating_Seeds_cDNA_P | − |
| 12385291 | 108464 | At_Germinating_Seeds_cDNA_P | − |
| 12385291 | 108594 | At_Ler-rhl_Root_DNA_P | − |
| 12385291 | 108595 | At_Ler-pi_Ovule_cDNA_P | − |
| 12385291 | 20000069 | At_100uM_ABA_Mutants_cDNA_P | − |
| 12385291 | 20000072 | At_100uM_ABA_Mutants_cDNA_P | − |
| 12385291 | 20000086 | At_100uM_ABA_Mutants_cDNA_P | − |
| 12385291 | 20000087 | At_100uM_ABA_Mutants_cDNA_P | − |
| 12385291 | 20000171 | At_42deg_Heat_P | − |
| 12385291 | 20000179 | At_Germinating_Seeds_P | − |
| 12385291 | 20000180 | At_Germinating_Seeds_P | − |
| 12385291 | 20000185 | At_Roots_P | − |
| 12385291 | 20000326 | At_Pollen_P | − |
| 12385291 | 20000437 | At_Drought_P | − |
| 12385291 | 20000439 | At_Roots_P | − |
| 12385291 | 20000453 | At_100uM_ABA_P | − |
| 12385291 | 20000794 | At_Petals_P | − |
| 12385291 | 20001247 | At_Far-red-induction_P | − |
| 12385291 | 20001248 | At_Far-red-induction_P | − |
| 12385291 | 20001450 | At_Far-red-induction_P | − |
| 12385291 | 20001451 | At_Far-red-induction_P | − |
| 12385291 | 20001554 | At_Drought_Soil_Dry_P | − |
| 12385291 | 20001555 | At_Drought_Soil_Dry_P | − |
| 12385291 | 20001556 | At_Drought_Soil_Dry_P | − |
| 12395532 | 108434 | At_Root_Tips_cDNA_P | − |
| 12395532 | 108461 | At_Germinating_Seeds_cDNA_P | − |
| 12395532 | 108462 | At_Germinating_Seeds_cDNA_P | − |
| 12395532 | 108561 | At_100uM_ABA_cDNA_P | − |
| 12395532 | 108575 | At_Wounding_cDNA_P | − |
| 12395532 | 108577 | At_42deg_Heat_cDNA_P | − |
| 12395532 | 108606 | At_100uM_ABA_cDNA_P | − |
| 12395532 | 20000046 | At_CS237-vs-Columbia_cDNA_P | − |
| 12395532 | 20000070 | At_100uM_ABA_Mutants_cDNA_P | − |
| 12395532 | 20000089 | At_2mM_SA_CS3726-Columbia_cDNA_P | − |
| 12395532 | 20000144 | At_42deg_Heat_cDNA_P | − |
| 12395532 | 20000171 | At_42deg_Heat_P | − |
| 12395532 | 20000184 | At_Shoots_P | − |
| 12395532 | 20000185 | At_Roots_P | − |
| 12395532 | 20000234 | At_Siliques_P | − |
| 12395532 | 20000236 | At_Siliques_P | − |
| 12395532 | 20000264 | At_Open_Flower_P | − |
| 12395532 | 20000265 | At_Open_Flower_P | − |
| 12395532 | 20000268 | At_100mM_NaCl_P | − |
| 12395532 | 20000286 | At_Open_Flower_P | − |
| 12395532 | 20000326 | At_Pollen_P | − |
| 12395532 | 20000437 | At_Drought_P | − |
| 12395532 | 20000438 | At_Shoots_P | − |
| 12395532 | 20000439 | At_Roots_P | − |
| 12395532 | 20000495 | At_Guard_Cells_P | − |
| 12395532 | 20000506 | At_Wounding_P | − |
| 12395532 | 20000573 | At_100uM_ABA_Mutants_P | − |
| 12395532 | 20000574 | At_100uM_ABA_Mutants2_P | − |
| 12395532 | 20000794 | At_Petals_P | − |
| 12395532 | 20001554 | At_Drought_Soil_Dry_P | − |
| 12395532 | 20001760 | At_50mM_NH4NO3_L-to-H_P | − |
| 12575820 | 108579 | At_4deg_Cold_cDNA_P | − |
| 12575820 | 108590 | At_15mM_NH4NO3_L-to-H_cDNA_P | − |
| 12575820 | 20000326 | At_Pollen_P | − |
| 12575820 | 20000439 | At_Roots_P | − |
| 12575820 | 20000495 | At_Guard_Cells_P | − |
| 12575820 | 20001248 | At_Far-red-induction_P | − |
| 12575820 | 20001557 | At_Drought_Soil_Dry_P | − |
| 12600234 | 20001248 | At_Far-red-induction_P | − |
| 12603755 | 20000326 | At_Pollen_P | − |
| 12640578 | 20000227 | At_Root-Tips-vs-Tops_P | − |
| 12640578 | 20000264 | At_Open_Flower_P | − |
| 12640578 | 20000265 | At_Open_Flower_P | − |
| 12640578 | 20000286 | At_Open_Flower_P | − |
| 12640578 | 20000326 | At_Pollen_P | − |
| 12640578 | 20000451 | At_CS6879_Shoots-Roots_P | − |
| 12640578 | 20000794 | At_Petals_P | − |
| 12647555 | 108473 | At_Drought_Flowers_cDNA_P | − |
| 12647555 | 108572 | At_Drought_cDNA_P | − |
| 12647555 | 108573 | At_Drought_cDNA_P | − |
| 12647555 | 108577 | At_42deg_Heat_P | − |
| 12647555 | 108668 | At_2mM_SA_cDNA_P | − |
| 12647555 | 20000111 | At_42deg_Heat_cDNA_P | − |
| 12647555 | 20000173 | At_42deg_Heat_P | − |
| 12647555 | 20000185 | At_Roots_P | − |
| 12647555 | 20000236 | At_Siliques_P | − |
| 12647555 | 20000268 | At_100mM_NaCl_P | − |
| 12647555 | 20000436 | At_Drought_P | − |
| 12647555 | 20000437 | At_Drought_P | − |
| 12647555 | 20000439 | At_Roots_P | − |
| 12647555 | 20000451 | At_CS6879_Shoots-Roots_P | − |
| 12647555 | 20001554 | At_Drought_Soil_Dry_P | − |
| 12647555 | 20001555 | At_Drought_Soil_Dry_P | − |
| 12647555 | 20001556 | At_Drought_Soil_Dry_P | − |
| 12649228 | 108434 | At_Root_Tips_cDNA_P | − |
| 12649228 | 20000326 | At_Pollen_P | − |
| 12658070 | 20000439 | At_Roots_P | − |
| 12721583 | 20000173 | At_42deg_Heat_P | − |
| 12721583 | 20000265 | At_Open_Flower_P | − |
| 12721583 | 20000458 | At_42deg_Heat_P | − |
| 12721583 | 20000794 | At_Petals_P | − |
| 12721583 | 20001555 | At_Drought_Soil_Dry_P | − |
| 12721583 | 20001556 | At_Drought_Soil_Dry_P | − |
| 12721583 | 20001557 | At_Drought_Soil_Dry_P | − |
| 12721583 | 20001558 | At_Drought_Soil_Dry_P | − |
| 12721583 | 20001559 | At_Drought_Soil_Dry_P | − |
| 12721583 | 20001560 | At_Drought_Soil_Dry_P | − |
| 13593439 | 20000173 | At_42deg_Heat_P | − |
| 13593439 | 20000184 | At_Shoots_P | − |
| 13593439 | 20000185 | At_Roots_P | − |
| 13593439 | 20001247 | At_Far-red-induction_P | − |
| 13593439 | 20001248 | At_Far-red-induction_P | − |
| 13593439 | 20001560 | At_Drought_Soil_Dry_P | − |
| 13612380 | 108434 | At_Root_Tips_cDNA_P | − |
| 13612380 | 108577 | At_42deg_Heat_cDNA_P | − |
| 13612380 | 108606 | At_100uM_ABA_cDNA_P | − |
| 13612380 | 20000046 | At_CS237-vs-Columbia_cDNA_P | − |
| 13612380 | 20000111 | At_42deg_Heat_cDNA_P | − |
| 13612380 | 20000113 | At_42deg_Heat_cDNA_P | − |
| 13612380 | 20000144 | At_42deg_Heat_cDNA_P | − |
| 13612380 | 20000166 | At_100uM_ABA_P | − |
| 13612380 | 20000169 | At_100uM_ABA_P | − |
| 13612380 | 20000173 | At_42deg_Heat_P | − |
| 13612380 | 20000179 | At_Germinating_Seeds_P | − |
| 13612380 | 20000180 | At_Germinating_Seeds_P | − |
| 13612380 | 20000184 | At_Shoots_P | − |
| 13612380 | 20000185 | At_Roots_P | − |
| 13612380 | 20000234 | At_Siliques_P | − |
| 13612380 | 20000236 | At_Siliques_P | − |
| 13612380 | 20000264 | At_Open_Flower_P | − |
| 13612380 | 20000265 | At_Open_Flower_P | − |
| 13612380 | 20000286 | At_Open_Flower_P | − |
| 13612380 | 20000436 | At_Drought_P | − |
| 13612380 | 20000438 | At_Shoots_P | − |
| 13612380 | 20000439 | At_Roots_P | − |
| 13612380 | 20000458 | At_42deg_Heat_P | − |
| 13612380 | 20000495 | At_Guard_Cells_P | − |
| 13612380 | 20000573 | At_100uM_ABA_Mutants_P | − |
| 13612380 | 20000574 | At_100uM_ABA_Mutants_P | − |
| 13612380 | 20000794 | At_Petals_P | − |
| 13612380 | 20001555 | At_Drought_Soil_Dry_P | − |

TABLE 4

| Utility Section | Short_Name |
|---|---|
| Nitric Oxide Responsive | At_5mM_NaNP |
| Reproductive and Seed & | At_Ler-pi_Ovule |
| Fruit Development | |
| | At_Caf_Knockout |
| | At_Siliques |
| | At_Open_Flower |
| | At_Petals |
| | At_Pollen |
| | At_ap2_floral_buds |
| Roots | At_Roots |
| | At_Root_Tips |
| | At_Ler-rhl_Root |
| Salicylic Acid | At_2mM_SA |
| | At_2mM_SA_CS3726-Columbia |
| Wounding | At_Wounding |
| Leaves, Shoots, Meristem | At_Shoots |
| | At_Shoot_Apices |
| Salt | At_100mM_NaCl |
| Guard Cells | At_Guard_Cells |
| ABA, Drought, Germination | At_100uM_ABA_Mutants |
| | At_100uM_ABA |
| | At_Drought_Soil_Dry |
| | At_Germinating_Seeds |
| Nitrogen | At_50mM_NH4NO3_L-to-H |
| | At_20uM_KNO3_H-to-L |
| | At_15mM_NH4NO3_L-to-H |
| Cold | At_4deg_Cold |
| Brassinosteroid Responsive | At_1uM_BR-BRZ |
| PEG | At_10%_PEG |
| Shade | At_Far-red-induction |
| Viability, Reproduction, | At_Line_Comparisons |
| Germination, Seed & | |
| Fruit Development | |
| Roots, Leaves, Shoots, Meristem | At_Root-Tips-vs-Tops |
| | At_CS6879_Shoots-Roots |
| Methyl Jasmonate | At_0.001%_MeJA |
| Hormone Responsive | At_CS237-vs-Columbia |

What is claimed is:

1. An isolated nucleic acid molecule comprising:
a) a nucleic acid having a nucleotide sequence which encodes an amino acid sequence exhibiting at least 85% sequence identity to an amino acid sequence in TABLE 1;
b) a nucleic acid which is a complement of a nucleotide sequence according to paragraph (a);
c) a nucleic acid which is the reverse of the nucleotide sequence according to subparagraph (a), such that the reverse nucleotide sequence has a sequence order which is the reverse of the sequence order of the nucleotide sequence according to subparagraph (a); or
d) a nucleic acid capable of hybridizing to a nucleic acid according to any one of paragraphs (a)-(c), under conditions that permit formation of a nucleic acid duplex at a temperature from about 40° C. and 48° C. below the melting temperature of the nucleic acid duplex.

2. The isolated nucleic acid molecule according to claim 1, which has the nucleotide sequence according to any sequence in TABLE 1.

3. The isolated nucleic acid molecule according to claim 1, wherein said amino acid sequence comprises any polypeptide sequence in TABLE 1.

4. A vector construct comprising:
a) a first nucleic acid having a regulatory sequence capable of causing transcription and/or translation in a plant; and
b) a second nucleic acid having the sequence of the isolated nucleic acid molecule according to any one of claims 1-3;
wherein said first and second nucleic acids are operably linked and wherein said second nucleic acid is heterologous to any element in said vector construct.

5. The vector construct according to claim 4, wherein said first nucleic acid is native to said second nucleic acid.

6. The vector construct according to claim 4, wherein said first nucleic acid is heterologous to said second nucleic acid.

7. A host cell comprising an isolated nucleic acid molecule according to any one of claims 1-3 wherein said nucleic acid molecule is flanked by exogenous sequence.

8. A host cell comprising a vector construct according to any one of claim 4.

9. An isolated polypeptide comprising an amino acid sequence exhibiting at least 85% sequence identity of an amino acid sequence of Table 1.

10. A method of introducing an isolated nucleic acid into a host cell comprising:
a) providing an isolated nucleic acid molecule according to any one of claims 1-3; and
b) contacting said isolated nucleic with said host cell under conditions that permit insertion of said nucleic acid into said host cell.

11. A method of transforming a host cell which comprises contacting a host cell with a vector construct according to any one of claims 4.

12. A method for detecting a nucleic acid in a sample which comprises:
a) providing an isolated nucleic acid molecule according to any one of claims 1-3;
b) contacting said isolated nucleic acid molecule with a sample under conditions which permit a comparison of the sequence of said isolated nucleic acid molecule with the sequence of DNA in said sample; and
c) analyzing the result of said comparison.

13. A plant, plant cell, plant material or seed of a plant which comprises a nucleic acid molecule according to any one of claims 1-3 which is exogenous or heterologous to said plant or plant cell.

14. A plant, plant cell, plant material or seed of a plant which comprises a vector construct according to any one of claims 4.

15. A plant which has been regenerated from a plant cell or seed according to claims 13.
